(19) United States
(12) Patent Application Publication
(10) Pub. No.: US 2025/0270527 A1
(43) Pub. Date: Aug. 28, 2025

(54) NUCLEOBASE EDITORS AND USES THEREOF

(71) Applicants: David R. Liu, Cambridge, MA (US); Alexis Christine Komor, Cambridge, MA (US); Holly A. Rees, Cambridge, MA (US); Yongjoo Kim, Cambridge, MA (US)

(72) Inventors: David R. Liu, Cambridge, MA (US); Alexis Christine Komor, Cambridge, MA (US); Holly A. Rees, Cambridge, MA (US); Yongjoo Kim, Cambridge, MA (US)

(73) Assignee: President and Fellows of Harvard College, Cambridge, MA (US)

(21) Appl. No.: 19/202,976

(22) Filed: May 8, 2025

Related U.S. Application Data
(63) Continuation of application No. 17/527,011, filed on Nov. 15, 2021, now Pat. No. 12,344,869.
Application No. 17/527,011 is a continuation of application No. 15/960,171, filed on Apr. 23, 2018, now Pat. No. 11,214,780.
Application No. 15/960,171 is a continuation of application No. PCT/US2016/058344, filed on Oct. 22, 2016.
Application No. 15/960,171 is a continuation of application No. 15/331,852, filed on Oct. 22, 2016, now Pat. No. 10,167,457.
(60) Provisional application No. 62/408,686, filed on Oct. 14, 2016.
(60) Provisional application No. 62/398,490, filed on Sep. 22, 2016.
(60) Provisional application No. 62/370,700, filed on Aug. 3, 2016.
(60) Provisional application No. 62/357,352, filed on Jun. 30, 2016.
(60) Provisional application No. 62/357,332, filed on Jun. 30, 2016.
(60) Provisional application No. 62/322,178, filed on Apr. 13, 2016.
(60) Provisional application No. 62/311,763, filed on Mar. 22, 2016.
(60) Provisional application No. 62/279,346, filed on Jan. 15, 2016.
(60) Provisional application No. 62/245,828, filed on Oct. 23, 2015.
(60) Provisional application No. 62/408,686, filed on Oct. 14, 2016.
(60) Provisional application No. 62/398,490, filed on Sep. 22, 2016.
(60) Provisional application No. 62/370,700, filed on Aug. 3, 2016.
(60) Provisional application No. 62/357,352, filed on Jun. 30, 2016.
(60) Provisional application No. 62/357,332, filed on Jun. 30, 2016.
(60) Provisional application No. 62/322,178, filed on Apr. 13, 2016.
(60) Provisional application No. 62/311,763, filed on Mar. 22, 2016.
(60) Provisional application No. 62/279,346, filed on Jan. 15, 2016.
(60) Provisional application No. 62/245,828, filed on Oct. 23, 2015.

(51) Int. Cl. C12N 9/22; C07K 14/32; C12N 9/24; C12N 9/78; C12N 15/11; C12N 15/90

(52) U.S. Cl. CPC C12N 9/22; C07K 14/32; C12N 9/2497; C12N 9/78; C12N 15/11; C12N 15/907; C12Y 305/04005; C07K 2319/00; C07K 2319/09; C07K 2319/80; C12N 2310/20; C12N 2800/80

(57) ABSTRACT

Some aspects of this disclosure provide strategies, systems, reagents, methods, and kits that are useful for the targeted editing of nucleic acids, including editing a single site within the genome of a cell or subject, e.g., within the human genome. In some embodiments, fusion proteins of Cas9 and nucleic acid editing proteins or protein domains, e.g., deaminase domains, are provided. In some embodiments, methods for targeted nucleic acid editing are provided. In some embodiments, reagents and kits for the generation of targeted nucleic acid editing proteins, e.g., fusion proteins of Cas9 and nucleic acid editing proteins or domains, are provided.

### RELATED APPLICATIONS

This application is a continuation of and claims priority under 35 U.S.C. § 120 to U.S. application Ser. No. 15/960,171, filed Apr. 23, 2018, which is a continuation of and claims priority under 35 U.S.C. § 120 to international PCT Application, PCT/US2016/058344, filed Oct. 22, 2016, and is a continuation of and claims priority under 35 U.S.C. § 120 to U.S. application Ser. No. 15/331,852, filed Oct. 22, 2016, which claims priority under 35 U.S.C. § 119(e) to U.S. provisional patent applications, U.S. Ser. No. 62/245,828 filed Oct. 23, 2015, U.S. Ser. No. 62/279,346 filed Jan. 15, 2016, U.S. Ser. No. 62/311,763 filed Mar. 22, 2016, U.S. Ser. No. 62/322,178 filed Apr. 13, 2016, U.S. Ser. No. 62/357,352 filed Jun. 30, 2016, U.S. Ser. No. 62/370,700 filed Aug. 3, 2016, U.S. Ser. No. 62/398,490 filed Sep. 22, 2016, U.S. Ser. No. 62/408,686 filed Oct. 14, 2016, and U.S. Ser. No. 62/357,332 filed Jun. 30, 2016; each of which is incorporated herein by reference.

### GOVERNMENT SUPPORT

This invention was made with Government support under grant number EB022376 (formerly GM065400) awarded by the National Institutes of Health. The Government has certain rights in the invention.

### REFERENCE TO A SEQUENCE LISTING SUBMITTED AS A TEXT FILE VIA EFS-WEB

This application contains a Sequence Listing which has been submitted in ASCII format via EFS-Web and is hereby incorporated by reference in its entirety. Said ASCII copy, created on Nov. 9, 2021, is named H082470213US10-SEQ-EPG and is 4,463,178 bytes in size.

### BACKGROUND OF THE INVENTION

Targeted editing of nucleic acid sequences, for example, the targeted cleavage or the targeted introduction of a specific modification into genomic DNA, is a highly promising approach for the study of gene function and also has the potential to provide new therapies for human genetic diseases.¹ An ideal nucleic acid editing technology possesses three characteristics: (1) high efficiency of installing the desired modification; (2) minimal off-target activity; and (3) the ability to be programmed to edit precisely any site in a given nucleic acid, e.g., any site within the human genome.² Current genome engineering tools, including engineered zinc finger nucleases (ZFNs),³ transcription activator like effector nucleases (TALENs),⁴ and most recently, the RNA-guided DNA endonuclease Cas9,⁵ effect sequence-specific DNA cleavage in a genome. This programmable cleavage can result in mutation of the DNA at the cleavage site via non-homologous end joining (NHEJ) or replacement of the DNA surrounding the cleavage site via homology-directed repair (HDR).^{6,7}

One drawback to the current technologies is that both NHEJ and HDR are stochastic processes that typically result in modest gene editing efficiencies as well as unwanted gene alterations that can compete with the desired alteration.⁸ Since many genetic diseases in principle can be treated by effecting a specific nucleotide change at a specific location in the genome (for example, a C to T change in a specific codon of a gene associated with a disease),⁹ the development of a programmable way to achieve such precision gene editing would represent both a powerful new research tool, as well as a potential new approach to gene editing-based human therapeutics.

### SUMMARY OF THE INVENTION

The clustered regularly interspaced short palindromic repeat (CRISPR) system is a recently discovered prokaryotic adaptive immune system¹⁰ that has been modified to enable robust and general genome engineering in a variety of organisms and cell lines.¹¹ CRISPR-Cas (CRISPR associated) systems are protein-RNA complexes that use an RNA molecule (sgRNA) as a guide to localize the complex to a target DNA sequence via base-pairing.¹² In the natural systems, a Cas protein then acts as an endonuclease to cleave the targeted DNA sequence.¹³ The target DNA sequence must be both complementary to the sgRNA, and also contain a “protospacer-adjacent motif” (PAM) at the 3′-end of the complementary region in order for the system to function.¹⁴

Among the known Cas proteins, S. pyogenes Cas9 has been mostly widely used as a tool for genome engineering.¹⁵ This Cas9 protein is a large, multi-domain protein containing two distinct nuclease domains. Point mutations can be introduced into Cas9 to abolish nuclease activity, resulting in a dead Cas9 (dCas9) that still retains its ability to bind DNA in a sgRNA-programmed manner.¹⁶ In principle, when fused to another protein or domain, dCas9 can target that protein to virtually any DNA sequence simply by co-expression with an appropriate sgRNA.

The potential of the dCas9 complex for genome engineering purposes is immense. Its unique ability to bring proteins to specific sites in a genome programmed by the sgRNA in theory can be developed into a variety of site-specific genome engineering tools beyond nucleases, including transcriptional activators, transcriptional repressors, histone-modifying proteins, integrases, and recombinases.¹¹ Some of these potential applications have recently been implemented through dCas9 fusions with transcriptional activators to afford RNA-guided transcriptional activators,^{17,18} transcriptional repressors,^{16,19,20} and chromatin modification enzymes.²¹ Simple co-expression of these fusions with a variety of sgRNAs results in specific expression of the target genes. These seminal studies have paved the way for the design and construction of readily programmable sequence-specific effectors for the precise manipulation of genomes.

Significantly, 80-90% of protein mutations responsible for human disease arise from the substitution, deletion, or insertion of only a single nucleotide.⁶ Most current strategies for single-base gene correction include engineered nucleases (which rely on the creation of double-strand breaks, DSBs, followed by stochastic, inefficient homology-directed repair, HDR), and DNA-RNA chimeric oligonucleotides.²² The latter strategy involves the design of a RNA/DNA sequence to base pair with a specific sequence in genomic DNA except at the nucleotide to be edited. The resulting mismatch is recognized by the cell's endogenous repair system and fixed, leading to a change in the sequence of either the chimera or the genome. Both of these strategies suffer from low gene editing efficiencies and unwanted gene alterations, as they are subject to both the stochasticity of HDR and the competition between HDR and non-homologous end-joining, NHEJ.²³⁻²⁵ HDR efficiencies vary according to the location of the target gene within the genome,²⁶ the state of the cell cycle,²⁷ and the type of cell/tissue.²⁸ The development of a direct, programmable way to install a specific type of base modification at a precise location in genomic DNA with enzyme-like efficiency and no stochasticity therefore represents a powerful new approach to gene editing-based research tools and human therapeutics.

Some aspects of the disclosure are based on the recognition that certain configurations of a dCas9 domain, and a cytidine deaminase domain fused by a linker are useful for efficiently deaminating target cytidine residues. Other aspects of this disclosure relate to the recognition that a nucleobase editing fusion protein with a cytidine deaminase domain fused to the N-terminus of a nuclease inactive Cas9 (dCas9) via a linker was capable of efficiently deaminating target nucleic acids in a double stranded DNA target molecule. See for example, Examples 3 and 4 below, which demonstrate that the fusion proteins, which are also referred to herein as base editors, generate less indels and more efficiently deaminate target nucleic acids than other base editors, such as base editors without a UGI domain. In some embodiments, the fusion protein comprises a nuclease-inactive Cas9 (dCas9) domain and an apolipoprotein B mRNA-editing complex 1 (APOBEC1) deaminase domain, where the deaminase domain is fused to the N-terminus of the dCas9 domain via a linker comprising the amino acid sequence SGSETPGTSESATPES (SEQ ID NO: 7). In some embodiments, the nuclease-inactive Cas9 (dCas9) domain of comprises the amino acid sequence set forth in SEQ ID NO: 263. In some embodiments, the deaminase is rat APOBEC1 (SEQ ID NO: 284). In some embodiments, the deaminase is human APOBEC1 (SEQ ID NO: 282). In some embodiments, the deaminase is pmCDA1 (SEQ ID NO: 5738). In some embodiments, the deaminase is human APOBEC3G (SEQ ID NO: 275). In some embodiments, the deaminase is a human APOBEC3G variant of any one of (SEQ ID NOs: 5739-5741).

Some aspects of the disclosure are based on the recognition that certain configurations of a dCas9 domain, and a cytidine deaminase domain fused by a linker are useful for efficiently deaminating target cytidine residues. Other aspects of this disclosure relate to the recognition that a nucleobase editing fusion protein with an apolipoprotein B mRNA-editing complex 1 (APOBEC1) deaminase domain fused to the N-terminus of a nuclease inactive Cas9 (dCas9) via a linker comprising the amino acid sequence SGSETPGTSESATPES (SEQ ID NO: 7) was capable of efficiently deaminating target nucleic acids in a double stranded DNA target molecule. In some embodiments, the fusion protein comprises a nuclease-inactive Cas9 (dCas9) domain and an apolipoprotein B mRNA-editing complex 1 (APOBEC1) deaminase domain, where the deaminase domain is fused to the N-terminus of the dCas9 domain via a linker comprising the amino acid sequence SGSETPGTSESATPES (SEQ ID NO: 7).

In some embodiments, the fusion protein comprises the amino acid residues 11-1629 of the amino acid sequence set forth in SEQ ID NO: 591. In some embodiments, the fusion protein comprises the amino acid sequence set forth in SEQ ID NO: 591. In some embodiments, the fusion protein comprises the amino acid sequence of any one of SEQ ID NOs: 5737, 5743, 5745, and 5746.

Some aspects of this disclosure provide strategies, systems, reagents, methods, and kits that are useful for the targeted editing of nucleic acids, including editing a single site within a subject's genome, e.g., a human's genome. In some embodiments, fusion proteins of Cas9 (e.g., dCas9, nuclease active Cas9, or Cas9 nickase) and deaminases or deaminase domains, are provided. In some embodiments, methods for targeted nucleic acid editing are provided. In some embodiments, reagents and kits for the generation of targeted nucleic acid editing proteins, e.g., fusion proteins of Cas9 and deaminases or deaminase domains, are provided.

Some aspects of this disclosure provide fusion proteins comprising a Cas9 protein as provided herein that is fused to a second protein (e.g., an enzymatic domain such as a cytidine deaminase domain), thus forming a fusion protein. In some embodiments, the second protein comprises an enzymatic domain, or a binding domain. In some embodiments, the enzymatic domain is a nuclease, a nickase, a recombinase, a deaminase, a methyltransferase, a methylase, an acetylase, an acetyltransferase, a transcriptional activator, or a transcriptional repressor domain. In some embodiments, the enzymatic domain is a nucleic acid editing domain. In some embodiments, the nucleic acid editing domain is a deaminase domain. In some embodiments, the deaminase is a cytosine deaminase or a cytidine deaminase. In some embodiments, the deaminase is an apolipoprotein B mRNA-editing complex (APOBEC) family deaminase. In some embodiments, the deaminase is an APOBEC1 deaminase. In some embodiments, the deaminase is an APOBEC2 deaminase. In some embodiments, the deaminase is an APOBEC3 deaminase. In some embodiments, the deaminase is an APOBEC3A deaminase. In some embodiments, the deaminase is an APOBEC3B deaminase. In some embodiments, the deaminase is an APOBEC3C deaminase. In some embodiments, the deaminase is an APOBEC3D deaminase. In some embodiments, the deaminase is an APOBEC3E deaminase. In some embodiments, the deaminase is an APOBEC3F deaminase. In some embodiments, the deaminase is an APOBEC3G deaminase. In some embodiments, the deaminase is an APOBEC3H deaminase. In some embodiments, the deaminase is an APOBEC4 deaminase. In some embodiments, the deaminase is an activation-induced deaminase (AID). It should be appreciated that the deaminase may be from any suitable organism (e.g., a human or a rat). In some embodiments, the deaminase is from a human, chimpanzee, gorilla, monkey, cow, dog, rat, or mouse. In some embodiments, the deaminase is rat APOBEC1 (SEQ ID NO: 284). In some embodiments, the deaminase is human APOBEC1 (SEQ ID NO: 282). In some embodiments, the deaminase is pmCDA1.

Some aspects of this disclosure provide fusion proteins comprising: (i) a nuclease-inactive Cas9 (dCas9) domain comprising the amino acid sequence of SEQ ID NO: 263; and (ii) an apolipoprotein B mRNA-editing complex 1 (APOBEC1) deaminase domain, wherein the deaminase domain is fused to the N-terminus of the dCas9 domain via a linker comprising the amino acid sequence of SGSETPGTSESATPES (SEQ ID NO: 7). In some embodiments, the deaminase is rat APOBEC1 (SEQ ID NO: 284). In some embodiments, the deaminase is human APOBEC1 (SEQ ID NO: 282). In some embodiments, the fusion protein comprises the amino acid sequence of SEQ ID NO: 591. In some embodiments, the fusion protein comprises the amino acid sequence of SEQ ID NO: 5737. In some embodiments, the deaminase is pmCDA1 (SEQ ID NO: 5738). In some embodiments, the deaminase is human APOBEC3G (SEQ ID NO: 275). In some embodiments, the deaminase is a human APOBEC3G variant of any one of SEQ ID NOs: 5739-5741.

Some aspects of this disclosure provide fusion proteins comprising: (i) a Cas9 nickase domain and (ii) an apolipoprotein B mRNA-editing complex 1 (APOBEC1) deaminase domain, wherein the deaminase domain is fused to the N-terminus of the Cas9 nickase domain. In some embodiments, the Cas9 nickase domain comprises a D10X mutation of the amino acid sequence provided in SEQ ID NO: 10, or a corresponding mutation in any of the amino acid sequences provided in SEQ ID NOs: 11-260, wherein X is any amino acid except for D. In some embodiments, the amino acid sequence of the Cas9 nickase domain comprises a D10A mutation of the amino acid sequence provided in SEQ ID NO: 10, or a corresponding mutation in any of the amino acid sequences provided in SEQ ID NOs: 11-260. In some embodiments, the amino acid sequence of the Cas9 nickase domain comprises a histidine at amino acid position 840 of the amino acid sequence provided in SEQ ID NO: 10, or a corresponding amino acid position in any of the amino acid sequences provided in SEQ ID NOs: 11-260. In some embodiments, the amino acid sequence of the Cas9 nickase domain comprises the amino acid sequence as set forth in SEQ ID NO: 267. In some embodiments, the deaminase is rat APOBEC1 (SEQ ID NO: 284). In some embodiments, the deaminase is human APOBEC1 (SEQ ID NO: 282). In some embodiments, the deaminase is pmCDA1.

Some aspects of this disclosure provide fusion proteins comprising: (i) a Cas9 nickase domain and (ii) an apolipoprotein B mRNA-editing complex 1 (APOBEC1) deaminase domain, wherein the deaminase domain is fused to the N-terminus of the Cas9 nickase domain. In some embodiments, the Cas9 nickase domain comprises a D10X mutation of the amino acid sequence provided in SEQ ID NO: 10, or a corresponding mutation in any of the amino acid sequences provided in SEQ ID NOs: 11-260, wherein X is any amino acid except for D. In some embodiments, the amino acid sequence of the Cas9 nickase domain comprises a D10A mutation of the amino acid sequence provided in SEQ ID NO: 10, or a corresponding mutation in any of the amino acid sequences provided in SEQ ID NOs: 11-260. In some embodiments, the amino acid sequence of the Cas9 nickase domain comprises a histidine at amino acid position 840 of the amino acid sequence provided in SEQ ID NO: 10, or a corresponding amino acid position in any of the amino acid sequences provided in SEQ ID NOs: 11-260. In some embodiments, the amino acid sequence of the Cas9 nickase domain comprises the amino acid sequence as set forth in SEQ ID NO: 267. In some embodiments, the deaminase is rat APOBEC1 (SEQ ID NO: 284). In some embodiments, the deaminase is human APOBEC1 (SEQ ID NO: 282). In some embodiments, the deaminase is pmCDA1.

Other aspects of this disclosure relate to the recognition that fusion proteins comprising a deaminase domain, a dCas9 domain and a uracil glycosylase inhibitor (UGI) domain demonstrate improved efficiency for deaminating target nucleotides in a nucleic acid molecule. Without wishing to be bound by any particular theory, cellular DNA-repair response to the presence of U:G heteroduplex DNA may be responsible for a decrease in nucleobase editing efficiency in cells. Uracil DNA glycosylase (UDG) catalyzes removal of U from DNA in cells, which may initiate base excision repair, with reversion of the U:G pair to a C:G pair as the most common outcome. As demonstrated herein, Uracil DNA Glycosylase Inhibitor (UGI) may inhibit human UDG activity. Without wishing to be bound by any particular theory, base excision repair may be inhibited by molecules that bind the single strand, block the edited base, inhibit UGI, inhibit base excision repair, protect the edited base, and/or promote “fixing” of the non-edited strand, etc. Thus, this disclosure contemplates fusion proteins comprising a dCas9-cytidine deaminase domain that is fused to a UGI domain.

In some embodiments, the fusion protein comprises a nuclease-inactive Cas9 (dCas9) domain; a nucleic acid editing domain; and a uracil glycosylase inhibitor (UGI) domain. In some embodiments, the dCas9 domain comprises a D10X mutation of the amino acid sequence provided in SEQ ID NO: 10, or a corresponding mutation in any of the amino acid sequences provided in SEQ ID NOs: 11-260, wherein X is any amino acid except for D. In some embodiments, the amino acid sequence of the dCas9 domain comprises a D10A mutation of the amino acid sequence provided in SEQ ID NO: 10, or a corresponding mutation in any of the amino acid sequences provided in SEQ ID NOs: 11-260. In some embodiments, the amino acid sequence of the dCas9 domain comprises an H840X mutation of the amino acid sequence provided in SEQ ID NO: 10, or a corresponding mutation in any of the amino acid sequences provided in SEQ ID NOs: 11-260, wherein X is any amino acid except for H. In some embodiments, the amino acid sequence of the dCas9 domain comprises an H840A mutation of the amino acid sequence provided in SEQ ID NO: 10, or a corresponding mutation in any of the amino acid sequences provided in SEQ ID NOs: 11-260. In some embodiments, the dCas9 domain comprises the amino acid sequence as set forth in SEQ ID NO: 263.

Further aspects of this disclosure relate to the recognition that fusion proteins using a Cas9 nickase as the Cas9 domain demonstrate improved efficiency for editing nucleic acids. For example, aspects of this disclosure relate to the recognition that fusion proteins comprising a Cas9 nickase, a deaminase domain and a UGI domain demonstrate improved efficiency for editing nucleic acids. For example, the improved efficiency for editing nucleotides is described below in the Examples section.

Some aspects of the disclosure are based on the recognition that any of the base editors provided herein are capable of modifying a specific nucleotide base without generating a significant proportion of indels. An “indel”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g. mutate or deaminate) a specific nucleotide within a nucleic acid, without generating a large number of insertions or deletions (i.e., indels) in the nucleic acid. In certain embodiments, any of the base editors provided herein are capable of generating a greater proportion of intended modifications (e.g., point mutations or deaminations) versus indels.

Some aspects of the disclosure are based on the recognition that any of the base editors provided herein are capable of efficiently generating an intended mutation, such as a point mutation, in a nucleic acid (e.g. a nucleic acid within a genome of a subject) without generating a significant number of unintended mutations, such as unintended point mutations.

In some embodiments, a fusion protein comprises a Cas9 nickase domain, a nucleic acid editing domain; and a uracil glycosylase inhibitor (UGI) domain. In some embodiments, the amino acid sequence of the Cas9 nickase domain comprises a D10X mutation of the amino acid sequence provided in SEQ ID NO: 10, or a corresponding mutation in any of the amino acid sequences provided in SEQ ID NOs: 11-260, wherein X is any amino acid except for D. In some embodiments, the amino acid sequence of the Cas9 nickase domain comprises a D10A mutation of the amino acid sequence provided in SEQ ID NO: 10, or a corresponding mutation in any of the amino acid sequences provided in SEQ ID NOs: 11-260. In some embodiments, the amino acid sequence of the Cas9 nickase domain comprises a histidine at amino acid position 840 of the amino acid sequence provided in SEQ ID NO: 10, or a corresponding amino acid position in any of the amino acid sequences provided in SEQ ID NOs: 11-260. In some embodiments, the amino acid sequence of the Cas9 nickase domain comprises the amino acid sequence as set forth in SEQ ID NO: 267.

In some embodiments, the deaminase domain of the fusion protein is fused to the N-terminus of the dCas9 domain or the Cas9 nickase. In some embodiments, the UGI domain is fused to the C-terminus of the dCas9 domain or the Cas9 nickase. In some embodiments, the dCas9 domain or the Cas9 nickase and the nucleic acid editing domain are fused via a linker. In some embodiments, the dCas9 domain or the Cas9 nickase and the UGI domain are fused via a linker.

In certain embodiments, linkers may be used to link any of the peptides or peptide domains of the invention. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may included functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some embodiments, the linker comprises the amino acid sequence (GGGGS)ₙ (SEQ ID NO: 5), (G)ₙ, (EAAAK)ₙ (SEQ ID NO: 6), (GGS)ₙ, (SGGS)ₙ (SEQ ID NO: 4288), SGSETPGTSESATPES (SEQ ID NO: 7), (XP)ₙ, or any combination thereof, wherein n is independently an integer between 1 and 30, and wherein X is any amino acid. In some embodiments, the linker comprises the amino acid sequence (GGS)ₙ, wherein n is 1, 3, or 7. In some embodiments, the linker comprises the amino acid sequence SGSETPGTSESATPES (SEQ ID NO: 7).

In some embodiments, the fusion protein comprises the structure [nucleic acid editing domain]-[optional linker sequence]-[dCas9 or Cas9 nickase]-[optional linker sequence]-[UGI]. In some embodiments, the fusion protein comprises the structure [nucleic acid editing domain]-[optional linker sequence]-[UGI]-[optional linker sequence]-[dCas9 or Cas9 nickase]; [UGI]-[optional linker sequence]-[nucleic acid editing domain]-[optional linker sequence]-[dCas9 or Cas9 nickase]; [UGI]-[optional linker sequence]-[dCas9 or Cas9 nickase]-[optional linker sequence]-[nucleic acid editing domain]; [dCas9 or Cas9 nickase]-[optional linker sequence]-[UGI]-[optional linker sequence]-[nucleic acid editing domain]; or [dCas9 or Cas9 nickase]-[optional linker sequence]-[nucleic acid editing domain]-[optional linker sequence]-[UGI].

In some embodiments, the nucleic acid editing domain comprises a deaminase. In some embodiments, the nucleic acid editing domain comprises a deaminase. In some embodiments, the deaminase is a cytidine deaminase. In some embodiments, the deaminase is an apolipoprotein B mRNA-editing complex (APOBEC) family deaminase. In some embodiments, the deaminase is an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, or an APOBEC4 deaminase. In some embodiments, the deaminase is an activation-induced deaminase (AID). In some embodiments, the deaminase is a Lamprey CDA1 (pmCDA1) deaminase.

In some embodiments, the deaminase is from a human, chimpanzee, gorilla, monkey, cow, dog, rat, or mouse. In some embodiments, the deaminase is from a human. In some embodiments the deaminase is from a rat. In some embodiments, the deaminase is a rat APOBEC1 deaminase comprising the amino acid sequence set forth in (SEQ ID NO: 284). In some embodiments, the deaminase is a human APOBEC1 deaminase comprising the amino acid sequence set forth in (SEQ ID NO: 282). In some embodiments, the deaminase is pmCDA1 (SEQ ID NO: 5738). In some embodiments, the deaminase is human APOBEC3G (SEQ ID NO: 275). In some embodiments, the deaminase is a human APOBEC3G variant of any one of (SEQ ID NOs: 5739-5741). In some embodiments, the deaminase is at least 80%, at least 85%, at least 90%, at least 92%, at least 95%, at least 96%, at least 97%, at least 98%, at least 99%, or at least 99.5% identical to any one of the amino acid sequences set forth in SEQ ID NOs: 266-284 or 5725-5741.

In some embodiments, the UGI domain comprises an amino acid sequence that is at least 80%, at least 85%, at least 90%, at least 92%, at least 95%, at least 96%, at least 97%, at least 98%, at least 99%, or at least 99.5% identical to SEQ ID NO: 600. In some embodiments, the UGI domain comprises the amino acid sequence as set forth in SEQ ID NO: 600.

Some aspects of this disclosure provide complexes comprising a Cas9 protein or a Cas9 fusion protein as provided herein, and a guide RNA bound to the Cas9 protein or the Cas9 fusion protein.

Some aspects of this disclosure provide methods of using the Cas9 proteins, fusion proteins, or complexes provided herein. For example, some aspects of this disclosure provide methods comprising contacting a DNA molecule (a) with a Cas9 protein or a fusion protein as provided herein and with a guide RNA, wherein the guide RNA is about 15-100 nucleotides long and comprises a sequence of at least 10 contiguous nucleotides that is complementary to a target sequence; or (b) with a Cas9 protein, a Cas9 fusion protein, or a Cas9 protein or fusion protein complex with a gRNA as provided herein.

Some aspects of this disclosure provide kits comprising a nucleic acid construct, comprising (a) a nucleotide sequence encoding a Cas9 protein or a Cas9 fusion protein as provided herein; and (b) a heterologous promoter that drives expression of the sequence of (a). In some embodiments, the kit further comprises an expression construct encoding a guide RNA backbone, wherein the construct comprises a cloning site positioned to allow the cloning of a nucleic acid sequence identical or complementary to a target sequence into the guide RNA backbone.

Some aspects of this disclosure provide polynucleotides encoding a Cas9 protein of a fusion protein as provided herein. Some aspects of this disclosure provide vectors comprising such polynucleotides. In some embodiments, the vector comprises a heterologous promoter driving expression of polynucleotide.

Some aspects of this disclosure provide cells comprising a Cas9 protein, a fusion protein, a nucleic acid molecule, and/or a vector as provided herein.

The description of exemplary embodiments of the reporter systems above is provided for illustration purposes only and not meant to be limiting. Additional reporter systems, e.g., variations of the exemplary systems described in detail above, are also embraced by this disclosure.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIG. 1 shows the deaminase activity of deaminases on single stranded DNA substrates. Single stranded DNA substrates using randomized PAM sequences (NNN PAM) were used as negative controls. Canonical PAM sequences used (NGG PAM)

FIG. 2 shows activity of Cas9:deaminase fusion proteins on single stranded DNA substrates.

FIG. 3 illustrates double stranded DNA substrate binding by Cas9:deaminase:sgRNA complexes.

FIG. 4 illustrates a double stranded DNA deamination assay.

FIG. 5 demonstrates that Cas9 fusions can target positions 3-11 of double-stranded DNA target sequences (numbered according to the schematic in FIG. 5). Upper Gel: 1 μM rAPOBEC1-GGS-dCas9, 125 nM dsDNA, 1 equivalent sgRNA. Mid Gel: 1 μM rAPOBEC1-(GGS)₃(SEQ ID NO: 596)-dCas9, 125 nM dsDNA, 1 equivalent sgRNA. Lower Gel: 1.85 μM rAPOBEC1-XTEN-dCas9, 125 nM dsDNA, 1 equivalent sgRNA.

FIG. 6 demonstrates that the correct guide RNA, e.g., the correct sgRNA, is required for deaminase activity.

FIG. 7 illustrates the mechanism of target DNA binding of in vivo target sequences by deaminase-dCas9:sgRNA complexes.

FIG. 8 shows successful deamination of exemplary disease-associated target sequences.

FIG. 9 shows in vitro C→T editing efficiencies using His6-rAPOBEC1-XTEN-dCas9.

FIG. 10 shows C→T editing efficiencies in HEK293T cells is greatly enhanced by fusion with UGI.

FIGS. 11A to 11C show NBE1 mediates specific, guide RNA-programmed C to U conversion in vitro. FIG. 11A: Nucleobase editing strategy. DNA with a target C (red) at a locus specified by a guide RNA (green) is bound by dCas9 (blue), which mediates the local denaturation of the DNA substrate. Cytidine deamination by a tethered APOBEC1 enzyme (orange) converts the target C to U. The resulting G:U heteroduplex can be permanently converted to an A:T base pair following DNA replication or repair. If the U is in the template DNA strand, it will also result in an RNA transcript containing a G to A mutation following transcription. FIG. 11B: Deamination assay showing an activity window of approximately five nucleotides. Following incubation of NBE1-sgRNA complexes with dsDNA substrates at 37° C. for 2 h, the 5′ fluorophore-labeled DNA was isolated and incubated with USER enzyme (uracil DNA glycosylase and endonuclease VIII) at 37° C. for 1 h to induce DNA cleavage at the site of any uracils. The resulting DNA was resolved on a denaturing polyacrylamide gel, and any fluorophore-linked strands were visualized. Each lane is labeled according to the position of the target C within the protospacer, or with “-” if no target C is present, counting the base distal from the PAM as position 1. FIG. 11C: Deaminase assay showing the sequence specificity and sgRNA-dependence of NBE1. The DNA substrate with a target C at position 7 was incubated with NBE1 as in FIG. 11B with either the correct sgRNA, a mismatched sgRNA, or no sgRNA. No C to U editing is observed with the mismatched sgRNA or with no sgRNA. The positive control sample contains a DNA sequence with a U synthetically incorporated at position 7.

FIGS. 12A to 12B show effects of sequence context and target C position on nucleobase editing efficiency in vitro. FIG. 12A: Effect of changing the sequence surrounding the target C on editing efficiency in vitro. The deamination yield of 80% of targeted strands (40% of total sequencing reads from both strands) for C₇ in the protospacer sequence 5′-TTATTTCGTGGATTTATTTA-3′(SEQ ID NO: 264) was defined as 1.0, and the relative deamination efficiencies of substrates containing all possible single-base mutations at positions 1-6 and 8-13 are shown. Values and error bars reflect the mean and standard deviation of two or more independent biological replicates performed on different days. FIG. 12B: Positional effect of each NC motif on editing efficiency in vitro. Each NC target motif was varied from positions 1 to 8 within the protospacer as indicated in the sequences shown on the right (the PAM shown in red, the protospacer plus one base 5′ to the protospacer are also shown). The percentage of total sequence reads containing T at each of the numbered target C positions following incubation with NBE1 is shown in the graph. Note that the maximum possible deamination yield in vitro is 50% of total sequencing reads (100% of targeted strands). Values and error bars reflect the mean and standard deviation of two or three independent biological replicates performed on different days. FIG. 12B depicts SEQ ID NOs: 285 through 292 from top to bottom, respectively.

FIGS. 13A to 13C show nucleobase editing in human cells. FIG. 13A: Protospacer (black) and PAM (red) sequences of the six mammalian cell genomic loci targeted by nucleobase editors. Target Cs are indicated with subscripted numbers corresponding to their positions within the protospacer. FIG. 13A depicts SEQ ID NOs: 293 through 298 from top to bottom, respectively. FIG. 13B: HEK293T cells were transfected with plasmids expressing NBE1, NBE2, or NBE3 and an appropriate sgRNA. Three days after transfection, genomic DNA was extracted and analyzed by high-throughput DNA sequencing at the six loci. Cellular C to T conversion percentages, defined as the percentage of total DNA sequencing reads with Ts at the target positions indicated, are shown for NBE1, NBE2, and NBE3 at all six genomic loci, and for wt Cas9 with a donor HDR template at three of the six sites (EMX1, HEK293 site 3, and HEK293 site 4). Values and error bars reflect the mean and standard deviation of three independent biological replicates performed on different days. FIG. 13C: Frequency of indel formation, calculated as described in the Methods, is shown following treatment of HEK293T cells with NBE2 and NBE3 for all six genomic loci, or with wt Cas9 and a single-stranded DNA template for HDR at three of the six sites (EMX1, HEK293 site 3, and HEK293 site 4). Values reflect the mean of at least three independent biological replicates performed on different days.

FIGS. 14A to 14C show NBE2- and NBE3-mediated correction of three disease-relevant mutations in mammalian cells. For each site, the sequence of the protospacer is indicated to the right of the name of the mutation, with the PAM highlighted in green and the base responsible for the mutation indicated in bold with a subscripted number corresponding to its position within the protospacer. The amino acid sequence above each disease-associated allele is shown, together with the corrected amino acid sequence following nucleobase editing in red. Underneath each sequence are the percentages of total sequencing reads with the corresponding base. Cells were nucleofected with plasmids encoding NBE2 or NBE3 and an appropriate sgRNA. Two days after nucleofection, genomic DNA was extracted and analyzed by HTS to assess pathogenic mutation correction. FIG. 14A: The Alzheimer's disease-associated APOE4 allele is converted to APOE3′ in mouse astrocytes by NBE3 in 11% of total reads (44% of nucleofected astrocytes). Two nearby Cs are also converted to Ts, but with no change to the predicted sequence of the resulting protein (SEQ ID NO: 299). FIG. 14B The cancer-associated p53 N239D mutation is corrected by NBE2 in 11% of treated human lymphoma cells (12% of nucleofected cells) that are heterozygous for the mutation (SEQ ID NO: 300). FIG. 14C The p53 Y163C mutation is corrected by NBE3 in 7.6% of nucleofected human breast cancer cells (SEQ ID NO: 301).

FIGS. 15A to 15D show effects of deaminase-dCas9 linker length and composition on nucleobase editing. Gel-based deaminase assay showing the deamination window of nucleobase editors with deaminase-Cas9 linkers of GGS (FIG. 15A), (GGS)₃ (SEQ ID NO: 596) (FIG. 15B), XTEN (FIG. 15C), or (GGS)₇ (SEQ ID NO: 597) (FIG. 15D). Following incubation of 1.85 μM editor-sgRNA complexes with 125 nM dsDNA substrates at 37° C. for 2 h, the dye-conjugated DNA was isolated and incubated with USER enzyme (uracil DNA glycosylase and endonuclease VIII) at 37° C. for an additional hour to cleave the DNA backbone at the site of any uracils. The resulting DNA was resolved on a denaturing polyacrylamide gel, and the dye-conjugated strand was imaged. Each lane is numbered according to the position of the target C within the protospacer, or with—if no target C is present. 8U is a positive control sequence with a U synthetically incorporated at position 8.

FIGS. 16A to 16B show NBE1 is capable of correcting disease-relevant mutations in vitro. FIG. 16A: Protospacer and PAM sequences (red) of seven disease-relevant mutations. The disease-associated target C in each case is indicated with a subscripted number reflecting its position within the protospacer. For all mutations except both APOE4 SNPs, the target C resides in the template (non-coding) strand. FIG. 16A depicts SEQ ID NOs: 302 through 308 from top to bottom, respectively. FIG. 16B: Deaminase assay showing each dsDNA oligonucleotide before (−) and after (+) incubation with NBE1, DNA isolation, and incubation with USER enzymes to cleave DNA at positions containing U. Positive control lanes from incubation of synthetic oligonucleotides containing U at various positions within the protospacer with USER enzymes are shown with the corresponding number indicating the position of the U.

FIG. 17 shows processivity of NBE1. The protospacer and PAM (red) of a 60-mer DNA oligonucleotide containing eight consecutive Cs is shown at the top. The oligonucleotide (125 nM) was incubated with NBE1 (2 μM) for 2 h at 37° C. The DNA was isolated and analyzed by high-throughput sequencing. Shown are the percent of total reads for the most frequent nine sequences observed. The vast majority of edited strands (>93%) have more than one C converted to T. This figure depicts SEQ ID NO: 309.

FIGS. 18A to 18H show the effect of fusing UGI to NBE1 to generate NBE2. FIG. 18A: Protospacer and PAM (red) sequences of the six mammalian cell genomic loci targeted with nucleobase editors. Editable Cs are indicated with labels corresponding to their positions within the protospacer. FIG. 18A depicts SEQ ID NOs: 293 through 298 from top to bottom, respectively. FIGS. 18B to 18G: HEK293T cells were transfected with plasmids expressing NBE1, NBE2, or NBE1 and UGI, and an appropriate sgRNA. Three days after transfection, genomic DNA was extracted and analyzed by high-throughput DNA sequencing at the six loci. Cellular C to T conversion percentages, defined as the percentage of total DNA sequencing reads with Ts at the target positions indicated, are shown for NBE1, NBE1 and UGI, and NBE2 at all six genomic loci. FIG. 18H: C to T mutation rates at 510 Cs surrounding the protospacers of interest for NBE1, NBE1 plus UGI on a separate plasmid, NBE2, and untreated cells are shown. The data show the results of 3,000,000 DNA sequencing reads from 1.5×106 cells. Values reflect the mean of at least two biological experiments conducted on different days.

FIG. 19 shows nucleobase editing efficiencies of NBE2 in U2OS and H1 EK293T cells. Cellular C to T conversion percentages by NBE2 are shown for each of the six targeted genomic loci in HEK293T cells and U2OS cells. HEK293T cells were transfected using lipofectamine 2000, and U2OS cells were nucleofected. U2OS nucleofection efficiency was 74%. Three days after plasmid delivery, genomic DNA was extracted and analyzed for nucleobase editing at the six genomic loci by HTS. Values and error bars reflect the mean and standard deviation of at least two biological experiments done on different days.

FIG. 20 shows nucleobase editing persists over multiple cell divisions. Cellular C to T conversion percentages by NBE2 are displayed at two genomic loci in HEK293T cells before and after passaging the cells. HEK293T cells were transfected using Lipofectamine 2000. Three days post transfection, the cells were harvested and split in half. One half was subjected to HTS analysis, and the other half was allowed to propagate for approximately five cell divisions, then harvested and subjected to HTS analysis.

FIG. 21 shows genetic variants from ClinVar that can be corrected in principle by nucleobase editing. The NCBI ClinVar database of human genetic variations and their corresponding phenotypes⁶⁸ was searched for genetic diseases that can be corrected by current nucleobase editing technologies. The results were filtered by imposing the successive restrictions listed on the left. The x-axis shows the number of occurrences satisfying that restriction and all above restrictions on a logarithmic scale.

FIG. 22 shows in vitro identification of editable Cs in six genomic loci. Synthetic 80-mers with sequences matching six different genomic sites were incubated with NBE1 then analyzed for nucleobase editing via HTS. For each site, the sequence of the protospacer is indicated to the right of the name of the site, with the PAM highlighted in red. Underneath each sequence are the percentages of total DNA sequencing reads with the corresponding base. A target C was considered as “editable” if the in vitro conversion efficiency is >10%. Note that maximum yields are 50% of total DNA sequencing reads since the non-targeted strand is not a substrate for nucleobase editing. This figure depicts SEQ ID NOs: 293 through 298 from top to bottom, respectively.

FIG. 23 shows activities of NBE1, NBE2, and NBE3 at EMX1 off-targets. HEK293T cells were transfected with plasmids expressing NBE1, NBE2, or NBE3 and a sgRNA matching the EMX1 sequence using Lipofectamine 2000. Three days after transfection, genomic DNA was extracted, amplified by PCR, and analyzed by high-throughput DNA sequencing at the on-target loci, plus the top ten known Cas9 off-target loci for the EMX1 sgRNA, as previously determined using the GUIDE-seq method⁵⁵. EMX1 off-target 5 locus did not amplify and is not shown. Sequences of the on-target and off-target protospacers and protospacer adjacent motifs (PAMs) are displayed. Cellular C to T conversion percentages, defined as the percentage of total DNA sequencing reads with T at each position of an original C within the protospacer, are shown for NBE1, NBE2, and NBE3. On the far right are displayed the total number of sequencing reads reported for each sequence. This figure depicts SEQ ID NOs: 293, and 310 through 318 from top to bottom, respectively.

FIG. 24 shows activities of NBE1, NBE2, and NBE3 at FANCF off-targets. HEK293T cells were transfected with plasmids expressing NBE1, NBE2, or NBE3 and a sgRNA matching the FANCF sequence using Lipofectamine 2000. Three days after transfection, genomic DNA was extracted, amplified by PCR, and analyzed by high-throughput DNA sequencing at the on-target loci, plus all of the known Cas9 off-target loci for the FANCF sgRNA, as previously determined using the GUIDE-seq method⁵⁵. Sequences of the on-target and off-target protospacers and protospacer adjacent motifs (PAMs) are displayed. Cellular C to T conversion percentages, defined as the percentage of total DNA sequencing reads with T at each position of an original C within the protospacer, are shown for NBE1, NBE2, and NBE3. On the far right are displayed the total number of sequencing reads reported for each sequence. This figure depicts SEQ ID NOs: 294 and 319 through 326 from top to bottom, respectively.

FIG. 25 shows activities of NBE1, NBE2, and NBE3 at HEK293 site 2 off-targets. HEK293T cells were transfected with plasmids expressing NBE1, NBE2, or NBE3 and a sgRNA matching the HEK293 site 2 sequence using Lipofectamine 2000. Three days after transfection, genomic DNA was extracted, amplified by PCR, and analyzed by high-throughput DNA sequencing at the on-target loci, plus all of the known Cas9 off-target loci for the HEK293 site 2 sgRNA, as previously determined using the GUIDE-seq method⁵⁵. Sequences of the on-target and off-target protospacers and protospacer adjacent motifs (PAMs) are displayed. Cellular C to T conversion percentages, defined as the percentage of total DNA sequencing reads with T at each position of an original C within the protospacer, are shown for NBE1, NBE2, and NBE3. On the far right are displayed the total number of sequencing reads reported for each sequence. This figure depicts SEQ ID NOs: 295, 327, and 328 from top to bottom, respectively.

FIG. 26 shows activities of NBE1, NBE2, and NBE3 at HEK293 site 3 off-targets. HEK293T cells were transfected with plasmids expressing NBE1, NBE2, or NBE3 and a sgRNA matching the HEK293 site 3 sequence using Lipofectamine 2000. Three days after transfection, genomic DNA was extracted, amplified by PCR, and analyzed by high-throughput DNA sequencing at the on-target loci, plus all of the known Cas9 off-target loci for the HEK293 site 3 sgRNA, as previously determined using the GUIDE-seq method⁵⁵. Sequences of the on-target and off-target protospacers and protospacer adjacent motifs (PAMs) are displayed. Cellular C to T conversion percentages, defined as the percentage of total DNA sequencing reads with T at each position of an original C within the protospacer, are shown for NBE1, NBE2, and NBE3. On the far right are displayed the total number of sequencing reads reported for each sequence. This figure depicts SEQ ID NOs: 296 and 659 through 663 from top to bottom, respectively.

FIG. 27 shows activities of NBE1, NBE2, and NBE3 at HEK293 site 4 off-targets. HEK293T cells were transfected with plasmids expressing NBE1, NBE2, or NBE3 and a sgRNA matching the HEK293 site 4 sequence using Lipofectamine 2000. Three days after transfection, genomic DNA was extracted, amplified by PCR, and analyzed by high-throughput DNA sequencing at the on-target loci, plus the top ten known Cas9 off-target loci for the HEK293 site 4 sgRNA, as previously determined using the GUIDE-seq method⁵⁵. Sequences of the on-target and off-target protospacers and protospacer adjacent motifs (PAMs) are displayed. Cellular C to T conversion percentages, defined as the percentage of total DNA sequencing reads with T at each position of an original C within the protospacer, are shown for NBE1, NBE2, and NBE3. On the far right are displayed the total number of sequencing reads reported for each sequence. This figure depicts SEQ ID NOs: 297 and 664 through 673 from top to bottom, respectively.

FIG. 28 shows non-target C mutation rates. Shown here are the C to T mutation rates at 2,500 distinct cytosines surrounding the six on-target and 34 off-target loci tested, representing a total of 14,700,000 sequence reads derived from approximately 1.8×106 cells.

FIGS. 29A to 29C show base editing in human cells. FIG. 29A shows possible base editing outcomes in mammalian cells. Initial editing resulted in a U:G mismatch. Recognition and excision of the U by uracil DNA glycosylase (UDG) initiated base excision repair (BER), which lead to reversion to the C:G starting state. BER was impeded by BE2 and BE3, which inhibited UDG. The U:G mismatch was also processed by mismatch repair (MMR), which preferentially repaired the nicked strand of a mismatch. BE3 nicked the non-edited strand containing the G, favoring resolution of the U:G mismatch to the desired U:A or T:A outcome. FIG. 29B shows HEK293T cells treated as described in the Materials and Methods in the Examples below. The percentage of total DNA sequencing read with Ts at the target positions indicated show treatment with BE1, BE2, or BE3, or for treatment with wt Cas9 with a donor HDR template. FIG. 29C shows frequency of indel formation following the treatment in FIG. 29B. Values are listed in FIG. 34. For FIGS. 29B and 29C, values and error bars reflect the mean and s.d. of three independent biological replicates performed on different days.

FIGS. 30A to 30B show BE3-mediated correction of two disease-relevant mutations in mammalian cells. The sequence of the protospacer is shown to the right of the mutation, with the PAM in blue and the target base in red with a subscripted number indicating its position within the protospacer. Underneath each sequence are the percentages of total sequencing reads with the corresponding base. Cells were treated as described in the Materials and Methods. FIG. 30A shows the Alzheimer's disease-associated APOE4 allele converted to APOE3r in mouse astrocytes by BE3 in 74.9% of total reads. Two nearby Cs were also converted to Ts, but with no change to the predicted sequence of the resulting protein. Identical treatment of these cells with wt Cas9 and donor ssDNA results in only 0.3% correction, with 26.1% indel formation. FIG. 30B shows the cancer associated p53 Y163C mutation corrected by BE3 in 7.6% of nucleofected human breast cancer cells with 0.7% indel formation. Identical treatment of these cells with wt Cas9 and donor ssDNA results in no mutation correction with 6.1% indel formation. This figure depicts SEQ ID NOs: 675 to 680 from top to bottom, respectively.

FIG. 31 shows activities of BE1, BE2, and BE3 at HEK293 site 2 off-targets. HEK293T cells were transfected with plasmids expressing BE1, BE2, or BE3 and a sgRNA matching the HEK293 site 2 sequence using Lipofectamine 2000. Three days after transfection, genomic DNA was extracted, amplified by PCR, and analyzed by high-throughput DNA sequencing at the on-target loci, plus all of the known Cas9 and dCas9 off-target loci for the HEK293 site 2 sgRNA, as previously determined by Joung and coworkers using the GUIDE-seq method (63), and Adli and coworkers using chromatin immunoprecipitation high-throughput sequencing (ChIP-seq) experiments (18). Sequences of the on-target and off-target protospacers and protospacer adjacent motifs (PAMs) are displayed. Cellular C to T conversion percentages, defined as the percentage of total DNA sequencing reads with T at each position of an original C within the protospacer, are shown for BE1, BE2, and BE3. On the far right are displayed the total number of sequencing reads reported, and the ChIP-seq signal intensity reported for each sequence. This figure depicts SEQ ID NOs: 681 to 688 from top to bottom, respectively.

FIG. 32 shows activities of BE1, BE2, and BE3 at HEK293 site 3 off-targets. HEK293T cells were transfected with plasmids expressing BE1, BE2, or BE3 and a sgRNA matching the HEK293 site 3 sequence using Lipofectamine 2000. Three days after transfection, genomic DNA was extracted, amplified by PCR, and analyzed by high-throughput DNA sequencing at the on-target loci, plus all of the known Cas9 off-target loci and the top five known dCas9 off-target loci for the HEK293 site 3 sgRNA, as previously determined by Joung and coworkers using the GUIDE-seq method⁵⁴, and using chromatin immunoprecipitation high-throughput sequencing (ChIP-seq) experiments⁶¹. Sequences of the on-target and off-target protospacers and protospacer adjacent motifs (PAMs) are displayed. Cellular C to T conversion percentages, defined as the percentage of total DNA sequencing reads with T at each position of an original C within the protospacer, are shown for BE1, BE2, and BE3. On the far right are displayed the total number of sequencing reads reported, and the ChIP-seq signal intensity reported for each sequence. This figure depicts SEQ ID NOs: 689 to 699 from top to bottom, respectively.

FIG. 33 shows activities of BE1, BE2, and BE3 at HEK293 site 4 off-targets. HEK293T cells were transfected with plasmids expressing BE1, BE2, or BE3 and a sgRNA matching the HEK293 site 4 sequence using Lipofectamine 2000. Three days after transfection, genomic DNA was extracted, amplified by PCR, and analyzed by high-throughput DNA sequencing at the on-target loci, plus the top ten known Cas9 off-target loci and the top five known dCas9 off-target loci for the HEK293 site 4 sgRNA, as previously determined using the GUIDE-seq method⁵⁴, and using chromatin immunoprecipitation high-throughput sequencing (ChIP-seq) experiments 61. Sequences of the on-target and off-target protospacers and protospacer adjacent motifs (PAMs) are displayed. Cellular C to T conversion percentages, defined as the percentage of total DNA sequencing reads with T at each position of an original C within the protospacer, are shown for BE1, BE2, and BE3. On the far right are displayed the total number of sequencing reads reported, and the ChIP-seq signal intensity reported for each sequence. This figure depicts SEQ ID NOs: 700 to 712 from top to bottom, respectively.

FIG. 34 shows mutation rates of non-protospacer bases following BE3-mediated correction of the Alzheimer's disease-associated APOE4 allele to APOE3r in mouse astrocytes. The DNA sequence of the 50 bases on either side of the protospacer from FIG. 30A and FIG. 34B is shown with each base's position relative to the protospacer. The side of the protospacer distal to the PAM is designated with positive numbers, while the side that includes the PAM is designated with negative numbers, with the PAM shown in blue. Underneath each sequence are the percentages of total DNA sequencing reads with the corresponding base for untreated cells, for cells treated with BE3 and an sgRNA targeting the APOE4 C158R mutation, or for cells treated with BE3 and an sgRNA targeting the VEGFA locus. Neither BE3-treated sample resulted in mutation rates above those of untreated controls. This figure depicts SEQ ID NOs: 713 to 716 from top to bottom, respectively.

FIG. 35 shows mutation rates of non-protospacer bases following BE3-mediated correction of the cancer-associated p53 Y163C mutation in HCC1954 human cells. The DNA sequence of the 50 bases on either side of the protospacer from FIG. 30B and FIG. 39B is shown with each base's position relative to the protospacer. The side of the protospacer distal to the PAM is designated with positive numbers, while the side that includes the PAM is designated with negative numbers, with the PAM shown in blue. Underneath each sequence are the percentages of total sequencing reads with the corresponding base for untreated cells, for cells treated with BE3 and an sgRNA targeting the TP53 Y163C mutation, or for cells treated with BE3 and an sgRNA targeting the VEGFA locus. Neither BE3-treated sample resulted in mutational rates above those of untreated controls. This figure depicts SEQ ID NOs: 717 to 720 from top to bottom, respectively.

FIGS. 36A to 36F show the effects of deaminase, linker length, and linker composition on base editing. FIG. 36A shows a gel-based deaminase assay showing activity of rAPOBEC1, pmCDA1, hAID, hAPOBEC3G, rAPOBEC1-GGS-dCas9, rAPOBEC1-(GGS)₃(SEQ ID NO: 596)-dCas9, and dCas9-(GGS)₃(SEQ ID NO: 596)-rAPOBEC1 on ssDNA. Enzymes were expressed in a mammalian cell lysate-derived in vitro transcription-translation system and incubated with 1.8 μM dye-conjugated ssDNA and USER enzyme (uracil DNA glycosylase and endonuclease VIII) at 37° C. for 2 hours. The resulting DNA was resolved on a denaturing polyacrylamide gel and imaged. The positive control is a sequence with a U synthetically incorporated at the same position as the target C. FIG. 36B shows coomassie-stained denaturing PAGE gel of the expressed and purified proteins used in FIGS. 36C to 36F. FIGS. 36C to 36F show gel-based deaminase assay showing the deamination window of base editors with deaminase-Cas9 linkers of GGS (FIG. 36C), (GGS)₃ (SEQ ID NO: 596) (FIG. 36D), XTEN (FIG. 36E), or (GGS)₇ (SEQ ID NO: 597) (FIG. 36F). Following incubation of 1.85 μM deaminase-dCas9 fusions complexed with sgRNA with 125 nM dsDNA substrates at 37° C. for 2 hours, the dye-conjugated DNA was isolated and incubated with USER enzyme at 37° C. for 1 hour to cleave the DNA backbone at the site of any uracils. The resulting DNA was resolved on a denaturing polyacrylamide gel, and the dye-conjugated strand was imaged. Each lane is numbered according to the position of the target C within the protospacer, or with—if no target C is present. 8U is a positive control sequence with a U synthetically incorporated at position 8.

FIGS. 37A to 37C show BE1 base editing efficiencies are dramatically decreased in mammalian cells. FIG. 37A Protospacer (black and red) and PAM (blue) sequences of the six mammalian cell genomic loci targeted by base editors. Target Cs are indicated in red with subscripted numbers corresponding to their positions within the protospacer. FIG. 37B shows synthetic 80-mers with sequences matching six different genomic sites were incubated with BE1 then analyzed for base editing by HTS. For each site, the sequence of the protospacer is indicated to the right of the name of the site, with the PAM highlighted in blue. Underneath each sequence are the percentages of total DNA sequencing reads with the corresponding base. We considered a target C as “editable” if the in vitro conversion efficiency is >10%. Note that maximum yields are 50% of total DNA sequencing reads since the non-targeted strand is unaffected by BE1. Values are shown from a single experiment. FIG. 37C shows HEK293T cells were transfected with plasmids expressing BE1 and an appropriate sgRNA. Three days after transfection, genomic DNA was extracted and analyzed by high-throughput DNA sequencing at the six loci. Cellular C to T conversion percentages, defined as the percentage of total DNA sequencing reads with Ts at the target positions indicated, are shown for BE1 at all six genomic loci. Values and error bars of all data from HEK293T cells reflect the mean and standard deviation of three independent biological replicates performed on different days. FIG. 37A depicts SEQ ID NOs: 721 to 726 from top to bottom, respectively. FIG. 37B depicts SEQ ID NOs: 727 to 732 from top to bottom, respectively.

FIG. 38 shows base editing persists over multiple cell divisions. Cellular C to T conversion percentages by BE2 and BE3 are shown for HEK293 sites 3 and 4 in HEK293T cells before and after passaging the cells. HEK293T cells were nucleofected with plasmids expressing BE2 or BE3 and an sgRNA targeting HEK293 site 3 or 4. Three days after nucleofection, the cells were harvested and split in half. One half was subjected to HTS analysis, and the other half was allowed to propagate for approximately five cell divisions, then harvested and subjected to HTS analysis. Values and error bars reflect the mean and standard deviation of at least two biological experiments.

FIGS. 39A to 39C show non-target C/G mutation rates. Shown here are the C to T and G to A mutation rates at 2,500 distinct cytosines and guanines surrounding the six on-target and 34 off-target loci tested, representing a total of 14,700,000 sequence reads derived from approximately 1.8×10⁶ cells. FIGS. 39A and 39B show cellular non-target C to T and G to A conversion percentages by BE1, BE2, and BE3 are plotted individually against their positions relative to a protospacer for all 2,500 cytosines/guanines. The side of the protospacer distal to the PAM is designated with positive numbers, while the side that includes the PAM is designated with negative numbers. FIG. 39C shows average non-target cellular C to T and G to A conversion percentages by BE1, BE2, and BE3 are shown, as well as the highest and lowest individual conversion percentages.

FIGS. 40A to 40B show additional data sets of BE3-mediated correction of two disease-relevant mutations in mammalian cells. For each site, the sequence of the protospacer is indicated to the right of the name of the mutation, with the PAM highlighted in blue and the base responsible for the mutation indicated in red bold with a subscripted number corresponding to its position within the protospacer. The amino acid sequence above each disease-associated allele is shown, together with the corrected amino acid sequence following base editing in green. Underneath each sequence are the percentages of total sequencing reads with the corresponding base. Cells were nucleofected with plasmids encoding BE3 and an appropriate sgRNA. Two days after nucleofection, genomic DNA was extracted from the nucleofected cells and analyzed by HTS to assess pathogenic mutation correction. FIG. 40A shows the Alzheimer's disease-associated APOE4 allele is converted to APOE3r in mouse astrocytes by BE3 in 58.3% of total reads only when treated with the correct sgRNA. Two nearby Cs are also converted to Ts, but with no change to the predicted sequence of the resulting protein. Identical treatment of these cells with wt Cas9 and donor ssDNA results in 0.2% correction, with 26.7% indel formation. FIG. 40B shows the cancer-associated p53 Y163C mutation is corrected by BE3 in 3.3% of nucleofected human breast cancer cells only when treated with the correct sgRNA. Identical treatment of these cells with wt Cas9 and donor ssDNA results in no detectable mutation correction with 8.0% indel formation. FIGS. 40A to 40B depict SEQ ID NOs: 733 to 740 from top to bottom, respectively.

FIG. 41 shows a schematic representation of an exemplary USER (Uracil-Specific Excision Reagent) Enzyme-based assay, which may be used to test the activity of various deaminases on single-stranded DNA (ssDNA) substrates.

FIG. 42 is a schematic of the pmCDA-nCas9-UGI-NLS construct and its activity at the HeK-3 site relative to the base editor (rAPOBEC1) and the negative control (untreated).

FIG. 43 is a schematic of the pmCDA1-XTEN-nCas9-UGI-NLS construct and its activity at the HeK-3 site relative to the base editor (rAPOBEC1) and the negative control (untreated).

FIG. 44 shows the percent of total sequencing reads with target C converted to T using cytidine deaminases (CDA) or APOBEC.

FIG. 45 shows the percent of total sequencing reads with target C converted to A using deaminases (CDA) or APOBEC.

FIG. 46 shows the percent of total sequencing reads with target C converted to G using deaminases (CDA) or APOBEC.

FIG. 47 is a schematic of the huAPOBEC3G-XTEN-nCas9-UGI-NLS construct and its activity at the HeK-2 site relative to a mutated form (huAPOBEC3G*(D316R_D317R)-XTEN-nCas9-UGI-NLS, the base editor (rAPOBEC1) and the negative control (untreated).

FIG. 48 shows the schematic of the LacZ construct used in the selection assay of Example 7.

FIG. 49 shows reversion data from different plasmids and constructs.

FIG. 50 shows the verification of lacZ reversion and the purification of reverted clones.

FIG. 51 is a schematic depicting a deamination selection plasmid used in Example 7.

FIG. 52 shows the results of a chloramphenicol reversion assay (pmCDA1 fusion).

FIGS. 53A to 53B demonstrated DNA correction induction of two constructs.

FIG. 54 shows the results of a chloramphenicol reversion assay (huAPOBEC3G fusion).

FIG. 55 shows the activities of BE3 and HF-BE3 at EMX1 off-targets. The sequences, from top to bottom, correspond to SEQ ID NOs: 286-292, 299-301.

FIG. 56 shows on-target base editing efficiencies of BE3 and HF-BE3.

FIG. 57 is a graph demonstrating that mutations affect cytidine deamination with varying degrees. Combinations of mutations that each slightly impairs catalysis allow selective deamination at one position over others. The FANCF site was GGAATC₆C₇C₈TTC₁₁TGCAGCACCTGG (SEQ ID NO: 303).

FIG. 58 is a schematic depicting next generation base editors.

FIG. 59 is a schematic illustrating new base editors made from Cas9 variants.

FIG. 60 shows the base-edited percentage of different NGA PAM sites.

FIG. 61 shows the base-edited percentage of cytidines using NGCG PAM EMX (VRER BE3) and the C₁TC₃C₄C₅ATC₈AC₁₀ATCAACCGGT (SEQ ID NO: 304) spacer.

FIG. 62 shows the based-edited percentages resulting from different NNGRRT PAM sites.

FIG. 63 shows the based-edited percentages resulting from different NNHRRT PAM sites.

FIGS. 64A to 64C show the base-edited percentages resulting from different TTTN PAM sites using Cpf1 BE2. The spacers used were: TTTCCTC₃C₄C₅C₆C₇C₈C₉AC₁₁AGGTAGAACAT (FIG. 64A, SEQ ID NO: 305), TTTCC₁C₂TC₄TGTC₈C₉AC₁₁ACCCTCATCCTG (FIG. 64B, SEQ ID NO: 306), and TTTCC₁C2C₃AGTC₇C₈TC₁₀C₁₁AC₁₃AC₁₅C₁₆C₁₇TGAAAC (FIG. 64C, SEQ ID NO: 307).

FIG. 65 is a schematic depicting selective deamination as achieved through kinetic modulation of cytidine deaminase point mutagenesis.

FIG. 66 is a graph showing the effect of various mutations on the deamination window probed in cell culture with multiple cytidines in the spacer. The spacer used was: TGC₃C₄C₅C₆TC₈C₉C₁₀TC₁₂C₁₃C₁₄TGGCCC (SEQ ID NO: 308).

FIG. 67 is a graph showing the effect of various mutations on the deamination window probed in cell culture with multiple cytidines in the spacer. The spacer used was: AGAGC₅C₆C₇C₈C₉C₁₀C₁₁TC₁₃AAAGAGA (SEQ ID NO: 309).

FIG. 68 is a graph showing the effect of various mutations on the FANCF site with a limited number of cytidines. The spacer used was: GGAATC₆C₇C₈TTC₁₁TGCAGCACCTGG (SEQ ID NO: 303). Note that the triple mutant (W90Y, R126E, R132E) preferentially edits the cytidine at the sixth position.

FIG. 69 is a graph showing the effect of various mutations on the HEK3 site with a limited number of cytidines. The spacer used was: GGCC₄C₅AGACTGAGCACGTGATGG (SEQ ID NO: 310). Note that the double and triple mutants preferentially edit the cytidine at the fifth position over the cytidine in the fourth position.

FIG. 70 is a graph showing the effect of various mutations on the EMX1 site with a limited number of cytidines. The spacer used was: GAGTC₅C₆GAGCAGAAGAAGAAGGG (SEQ ID NO: 311). Note that the triple mutant only edits the cytidine at the fifth position, not the sixth.

FIG. 71 is a graph showing the effect of various mutations on the HEK2 site with a limited number of cytidines. The spacer used was: GAAC₄AC₆AAAGCATAGACTGCGGG (SEQ ID NO: 312).

FIG. 72 shows on-target base editing efficiencies of BE3 and BE3 comprising mutations W90Y R132E in immortalized astrocytes.

FIG. 73 depicts a schematic of three Cpf1 fusion constructs.

FIG. 74 shows a comparison of plasmid delivery of BE3 and HF-BE3 (EMX1, FANCF, and RNF2).

FIG. 75 shows a comparison of plasmid delivery of BE3 and HF-BE3 (HEK3 and HEK 4).

FIG. 76 shows off-target editing of EMX-1 at all 10 sites.

FIG. 77 shows deaminase protein lipofection to HEK cells using a GAGTCCGAGCAGAAGAAGAAG (SEQ ID NO: 313) spacer. The EMX-1 on-target and EMX-1 off target site 2 were examined.

FIG. 78 shows deaminase protein lipofection to HEK cells using a GGAATCCCTTCTGCAGCACCTGG (SEQ ID NO: 314) spacer. The FANCF on target and FANCF off target site 1 were examined.

FIG. 79 shows deaminase protein lipofection to HEK cells using a GGCCCAGACTGAGCACGTGA (SEQ ID NO: 315) spacer. The HEK-3 on target site was examined.

FIG. 80 shows deaminase protein lipofection to HEK cells using a GGCACTGCGGCTGGAGGTGGGGG (SEQ ID NO: 316) spacer. The HEK-4 on target, off target site 1, site 3, and site 4.

FIG. 81 shows the results of an in vitro assay for sgRNA activity for sgHR_13 (GTCAGGTCGAGGGTTCTGTC (SEQ ID NO: 317) spacer; C8 target: G51 to STOP), sgHR_14 (GGGCCGCAGTATCCTCACTC (SEQ ID NO: 318) spacer; C7 target; C7 target: Q68 to STOP), and sgHR_15 (CCGCCAGTCCCAGTACGGGA (SEQ ID NO: 319) spacer; C10 and C11 are targets: W239 or W237 to STOP).

FIG. 82 shows the results of an in vitro assay for sgHR_17 (CAACCACTGCTCAAAGATGC (SEQ ID NO: 320) spacer; C4 and C5 are targets: W410 to STOP), and sgHR_16 (CTTCCAGGATGAGAACACAG (SEQ ID NO: 321) spacer; C4 and C5 are targets: W273 to STOP).

FIG. 83 shows the direct injection of BE3 protein complexed with sgHR_13 in zebrafish embryos.

FIG. 84 shows the direct injection of BE3 protein complexed with sgHR_16 in zebrafish embryos.

FIG. 85 shows the direct injection of BE3 protein complexed with sgHR_17 in zebrafish embryos.

FIG. 86 shows exemplary nucleic acid changes that may be made using base editors that are capable of making a cytosine to thymine change.

FIG. 87 shows an illustration of apolipoprotein E (APOE) isoforms, demonstrating how a base editor (e.g., BE3) may be used to edit one APOE isoform (e.g., APOE4) into another APOE isoform (e.g., APOE3r) that is associated with a decreased risk of Alzheimer's disease.

FIG. 88 shows base editing of APOE4 to APOE3r in mouse astrocytes.

FIG. 89 shows base editing of PRNP to cause early truncation of the protein at arginine residue 37.

FIG. 90 shows that knocking out UDG (which UGI inhibits) dramatically improves the cleanliness of efficiency of C to T base editing.

FIG. 91 shows that use of a base editor with the nickase but without UGI leads to a mixture of outcomes, with very high indel rates.

FIGS. 92A to 92G show that SaBE3, SaKKH-BE3, VQR-BE3, EQR-BE3, and VRER-BE3 mediate efficient base editing at target sites containing non-NGG PAMs in human cells. FIG. 92A shows base editor architectures using S. pyogenes and S. aureus Cas9. FIG. 92B shows recently characterized Cas9 variants with alternate or relaxed PAM requirements. FIGS. 92C and 92D show HEK293T cells treated with the base editor variants shown as described in Example 12. The percentage of total DNA sequencing reads (with no enrichment for transfected cells) with C converted to T at the target positions indicated are shown. The PAM sequence of each target tested is shown below the X-axis. The charts show the results for SaBE3 and SaKKH-BE3 at genomic loci with NNGRRT PAMs (FIG. 92C), SaBE3 and SaKKH-BE3 at genomic loci with NNNRRT PAMs (FIG. 92D), VQR-BE3 and EQR-BE3 at genomic loci with NGAG PAMs (FIG. 92E), and with NGAH PAMs (FIG. 92F), and VRER-BE3 at genomic loci with NGCG PAMs (FIG. 92G). Values and error bars reflect the mean and standard deviation of at least two biological replicates.

FIGS. 93A to 93C demonstrate that base editors with mutations in the cytidine deaminase domain exhibit narrowed editing windows. FIGS. 93A to 93C show HEK293T cells transfected with plasmids expressing mutant base editors and an appropriate sgRNA. Three days after transfection, genomic DNA was extracted and analyzed by high-throughput DNA sequencing at the indicated loci. The percentage of total DNA sequencing reads (without enrichment for transfected cells) with C changed to T at the target positions indicated are shown for the EMX1 site, HEK293 site 3, FANCF site, HEK293 site 2, site A, and site B loci. FIG. 93A illustrates certain cytidine deaminase mutations which narrow the base editing window. See FIG. 98 for the characterization of additional mutations. FIG. 93B shows the effect of cytidine deaminase mutations which effect the editing window width on genomic loci. Combining beneficial mutations has an additive effect on narrowing the editing window. FIG. 93C shows that YE1-BE3, YE2-BE3, EE-BE3, and YEE-BE3 effect the product distribution of base editing, producing predominantly singly-modified products in contrast with BE3. Values and error bars reflect the mean and standard deviation of at least two biological replicates.

FIGS. 94A and 94B show genetic variants from ClinVar that in principle can be corrected by the base editors developed in this work. The NCBI ClinVar database of human genetic variations and their corresponding phenotypes was searched for genetic diseases that in theory can be corrected by base editing. FIG. 94A demonstrates improvement in base editing targeting scope among all pathogenic T→C mutations in the ClinVar database through the use of base editors with altered PAM specificities. The white fractions denote the proportion of pathogenic T→C mutations accessible on the basis of the PAM requirements of either BE3, or BE3 together with the five modified-PAM base editors developed in this work. FIG. 94B shows improvement in base editing targeting scope among all pathogenic T→C mutations in the ClinVar database through the use of base editors with narrowed activity windows. BE3 was assumed to edit Cs in positions 4-8 with comparable efficiency as shown in FIGS. 93A to 93C. YEE-BE3 was assumed to edit with C5>C6>C7>others preference within its activity window. The white fractions denote the proportion of pathogenic T→C mutations that can be edited BE3 without comparable editing of other Cs (left), or that can be edited BE3 or YEE-BE3 without comparable editing of other Cs (right).

FIGS. 95A and 95B show the effect of truncated guide RNAs on base editing window width. HEK293T cells were transfected with plasmids expressing BE3 and sgRNAs of different 5′ truncation lengths. The treated cells were analyzed as described in the Examples. FIG. 95A shows protospacer and PAM sequence (top, SEQ ID NO: 4270) and cellular C to T conversion percentages, defined as the percentage of total DNA sequencing reads with Ts at the target positions indicated, at a site within the EMX1 genomic locus. At this site, the base editing window was altered through the use of a 17-nt truncated gRNA. FIG. 95B shows protospacer and PAM sequences (top, SEQ ID NO: 4270) and cellular C to T conversion percentages, defined as the percentage of total DNA sequencing reads with Ts at the target positions indicated, at sites within the HEK site 3 and site 4 genomic loci. At these sites, no change in the base editing window was observed, but a linear decrease in editing efficiency for all substrate bases as the sgRNA is truncated was noted.

FIG. 96 shows the effect of APOBEC1-Cas9 linker lengths on base editing window width. HEK293T cells were transfected with plasmids expressing base editors with rAPOBEC1-Cas9 linkers of XTEN, GGS, (GGS)₃ (SEQ ID NO: 596), (GGS)₅ (SEQ ID NO: 4271), or (GGS)₇ (SEQ ID NO: 597) and an sgRNA. The treated cells were analyzed as described in the Examples. Cellular C to T conversion percentages, defined as the percentage of total DNA sequencing reads with Ts at the target positions indicated, are shown for the various base editors with different linkers.

FIGS. 97A to 97C show the effect of rAPOBEC mutations on base editing window width. FIG. 97C shows HEK293T cells transfected with plasmids expressing an sgRNA targeting either Site A or Site B and the BE3 point mutants indicated. The treated cells were analyzed as described in the Examples. All C's in the protospacer and within three basepairs of the protospacer are displayed and the cellular C to T conversion percentages are shown. The ‘editing window widths’, defined as the calculated number of nucleotides within which editing efficiency exceeds the half-maximal value, are displayed for all tested mutants.

FIG. 98 shows the effect of APOBEC1 mutation son product distributions of base editing in mammalian cells. HEK293T cells were transfected with plasmids expressing BE3 or its mutants and an appropriate sgRNAs. The treated cells were analyzed as described in the Examples. Cellular C to T conversion percentages, defined as the percentage of total DNA sequencing reads with Ts at the target positions indicated, are shown (left). Percent of total sequencing reads containing the C to T conversion is shown on the right. The BE3 point mutants do not significantly affect base editing efficiencies at HEK site 4, a site with only one target cytidine.

FIG. 99 shows a comparison of on-target editing plasma delivery in BE3 and HF-BE3.

FIG. 100 shows a comparison of on-target editing in protein and plasma delivery of BE3.

FIG. 101 shows a comparison of on-target editing in protein and plasma delivery of HF-BE3.

FIG. 102 shows that both lipofection and installing HF mutations decrease off-target deamination events. The diamond indicates no off targets were detected and the specificity ratio was set to 100.

FIG. 103 shows in vitro C to T editing on a synthetic substrate with Cs placed at even positions in the protospacer (NNNNTC₂TC₄TC₆TC₈TC₁₀TC₁₂TC₁₄TC₁₆TC₁₈TC₂₀NGG, SEQ ID NO: 4272).

FIG. 104 shows in vitro C to T editing on a synthetic substrate with Cs placed at odd positions in the protospacer (NNNNTC₂TC₄TC₆TC₈TC₁₀TC₁₂TC₁₄TC₁₆TC₁₈TC₂₀NGG, SEQ ID NO: 4272).

FIG. 105 includes two graphs depicting the specificity ratio of base editing with plasmid vs. protein delivery.

FIGS. 106A to 106B shows BE3 activity on non-NGG PAM sites. HEK293T cells were transfected with plasmids expressing BE3 and appropriate sgRNA. The treated cells were analyzed as described in the Examples. FIG. 106A shows BE3 activity on sites can be efficiently targeted by SaBE3 or SaKKH-BE3. BE3 shows low but significant activity on the NAG PAM. FIG. 106B shows BE3 has significantly reduced editing at sites with NGA or NGCG PAMs, in contrast to VQR-BE3 or VRER-BE3.

FIGS. 107A to 107B show the effect of APOBEC1 mutations on VQR-BE3 and SaKKH-BE3. HEK293T cells were transfected with plasmids expressing VQR-BE3, SaKKH-BE3 or its mutants and an appropriate sgRNAs. The treated cells were analyzed as described in the Methods. Cellular C to T conversion percentages, defined as the percentage of total DNA sequencing reads with Ts at the target positions indicated, are shown. FIG. 107A shows that the window-modulating mutations can be applied to VQR-BE3 to enable selective base editing at sites targetable by NGA PAM. FIG. 107B shows that, when applied to SaKKH-BE3, the mutations cause overall decrease in base editing efficiency without conferring base selectivity within the target window.

FIG. 108 shows a schematic representation of nucleotide editing. The following abbreviations are used: (MMR)—mismatch repair, (BE3 Nickase)—refers to base editor 3, which comprises a Cas9 nickase domain, (UGI)—uracil glycosylase inhibitor, UDG)—uracil DNA glycosylase, (APOBEC)—refers to an APOBEC cytidine deaminase.

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents. The term “Cas9” or “Cas9 nuclease” refers to an RNA-guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain, that is, the Cas9 is a nickase.

A nuclease-inactivated Cas9 protein may interchangeably be referred to as a “dCas9” protein (for nuclease-“dead” Cas9). Methods for generating a Cas9 protein (or a fragment thereof) having an inactive DNA cleavage domain are known (See, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

In some embodiments, the fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length. In some embodiments, wild type Cas9 corresponds to Cas9 from Streptococcus pyogenes (NCBI Reference Sequence: NC_017053.1, SEQ ID NO:1 (nucleotide); SEQ ID NO:2 (amino acid)).

(SEQ ID NO: 1)
ATGGATAAGAAATACTCAATAGGCTTAGATATCGGCACAAATAGC
GTCGGATGGGCGGTGATCACTGATGATTATAAGGTTCCGTCTAAA
AAGTTCAAGGTTCTGGGAAATACAGACCGCCACAGTATCAAAAAA
AATCTTATAGGGGCTCTTTTATTTGGCAGTGGAGAGACAGCGGAA
GCGACTCGTCTCAAACGGACAGCTCGTAGAAGGTATACACGTCGG
AAGAATCGTATTTGTTATCTACAGGAGATTTTTTCAAATGAGATG
GCGAAAGTAGATGATAGTTTCTTTCATCGACTTGAAGAGTCTTTT
TTGGTGGAAGAAGACAAGAAGCATGAACGTCATCCTATTTTTGGA
AATATAGTAGATGAAGTTGCTTATCATGAGAAATATCCAACTATC
TATCATCTGCGAAAAAAATTGGCAGATTCTACTGATAAAGCGGAT
TTGCGCTTAATCTATTTGGCCTTAGCGCATATGATTAAGTTTCGT
GGTCATTTTTTGATTGAGGGAGATTTAAATCCTGATAATAGTGAT
GTGGACAAACTATTTATCCAGTTGGTACAAATCTACAATCAATTA
TTTGAAGAAAACCCTATTAACGCAAGTAGAGTAGATGCTAAAGCG
ATTCTTTCTGCACGATTGAGTAAATCAAGACGATTAGAAAATCTC
ATTGCTCAGCTCCCCGGTGAGAAGAGAAATGGCTTGTTTGGGAAT
CTCATTGCTTTGTCATTGGGATTGACCCCTAATTTTAAATCAAAT
TTTGATTTGGCAGAAGATGCTAAATTACAGCTTTCAAAAGATACT
TACGATGATGATTTAGATAATTTATTGGCGCAAATTGGAGATCAA
TATGCTGATTTGTTTTTGGCAGCTAAGAATTTATCAGATGCTATT
TTACTTTCAGATATCCTAAGAGTAAATAGTGAAATAACTAAGGCT
CCCCTATCAGCTTCAATGATTAAGCGCTACGATGAACATCATCAA
GACTTGACTCTTTTAAAAGCTTTAGTTCGACAACAACTTCCAGAA
AAGTATAAAGAAATCTTTTTTGATCAATCAAAAAACGGATATGCA
GGTTATATTGATGGGGGAGCTAGCCAAGAAGAATTTTATAAATTT
ATCAAACCAATTTTAGAAAAAATGGATGGTACTGAGGAATTATTG
GTGAAACTAAATCGTGAAGATTTGCTGCGCAAGCAACGGACCTTT
GACAACGGCTCTATTCCCCATCAAATTCACTTGGGTGAGCTGCAT
GCTATTTTGAGAAGACAAGAAGACTTTTATCCATTTTTAAAAGAC
AATCGTGAGAAGATTGAAAAAATCTTGACTTTTCGAATTCCTTAT
TATGTTGGTCCATTGGCGCGTGGCAATAGTCGTTTTGCATGGATG
ACTCGGAAGTCTGAAGAAACAATTACCCCATGGAATTTTGAAGAA
GTTGTCGATAAAGGTGCTTCAGCTCAATCATTTATTGAACGCATG
ACAAACTTTGATAAAAATCTTCCAAATGAAAAAGTACTACCAAAA
CATAGTTTGCTTTATGAGTATTTTACGGTTTATAACGAATTGACA
AAGGTCAAATATGTTACTGAGGGAATGCGAAAACCAGCATTTCTT
TCAGGTGAACAGAAGAAAGCCATTGTTGATTTACTCTTCAAAACA
AATCGAAAAGTAACCGTTAAGCAATTAAAAGAAGATTATTTCAAA
AAAATAGAATGTTTTGATAGTGTTGAAATTTCAGGAGTTGAAGAT
AGATTTAATGCTTCATTAGGCGCCTACCATGATTTGCTAAAAATT
ATTAAAGATAAAGATTTTTTGGATAATGAAGAAAATGAAGATATC
TTAGAGGATATTGTTTTAACATTGACCTTATTTGAAGATAGGGGG
ATGATTGAGGAAAGACTTAAAACATATGCTCACCTCTTTGATGAT
AAGGTGATGAAACAGCTTAAACGTCGCCGTTATACTGGTTGGGGA
CGTTTGTCTCGAAAATTGATTAATGGTATTAGGGATAAGCAATCT
GGCAAAACAATATTAGATTTTTTGAAATCAGATGGTTTTGCCAAT
CGCAATTTTATGCAGCTGATCCATGATGATAGTTTGACATTTAAA
GAAGATATTCAAAAAGCACAGGTGTCTGGACAAGGCCATAGTTTA
CATGAACAGATTGCTAACTTAGCTGGCAGTCCTGCTATTAAAAAA
GGTATTTTACAGACTGTAAAAATTGTTGATGAACTGGTCAAAGTA
ATGGGGCATAAGCCAGAAAATATCGTTATTGAAATGGCACGTGAA
AATCAGACAACTCAAAAGGGCCAGAAAAATTCGCGAGAGCGTATG
AAACGAATCGAAGAAGGTATCAAAGAATTAGGAAGTCAGATTCTT
AAAGAGCATCCTGTTGAAAATACTCAATTGCAAAATGAAAAGCTC
TATCTCTATTATCTACAAAATGGAAGAGACATGTATGTGGACCAA
GAATTAGATATTAATCGTTTAAGTGATTATGATGTCGATCACATT
GTTCCACAAAGTTTCATTAAAGACGATTCAATAGACAATAAGGTA
CTAACGCGTTCTGATAAAAATCGTGGTAAATCGGATAACGTTCCA
AGTGAAGAAGTAGTCAAAAAGATGAAAAACTATTGGAGACAACTT
CTAAACGCCAAGTTAATCACTCAACGTAAGTTTGATAATTTAACG
AAAGCTGAACGTGGAGGTTTGAGTGAACTTGATAAAGCTGGTTTT
ATCAAACGCCAATTGGTTGAAACTCGCCAAATCACTAAGCATGTG
GCACAAATTTTGGATAGTCGCATGAATACTAAATACGATGAAAAT
GATAAACTTATTCGAGAGGTTAAAGTGATTACCTTAAAATCTAAA
TTAGTTTCTGACTTCCGAAAAGATTTCCAATTCTATAAAGTACGT
GAGATTAACAATTACCATCATGCCCATGATGCGTATCTAAATGCC
GTCGTTGGAACTGCTTTGATTAAGAAATATCCAAAACTTGAATCG
GAGTTTGTCTATGGTGATTATAAAGTTTATGATGTTCGTAAAATG
ATTGCTAAGTCTGAGCAAGAAATAGGCAAAGCAACCGCAAAATAT
TTCTTTTACTCTAATATCATGAACTTCTTCAAAACAGAAATTACA
CTTGCAAATGGAGAGATTCGCAAACGCCCTCTAATCGAAACTAAT
GGGGAAACTGGAGAAATTGTCTGGGATAAAGGGCGAGATTTTGCC
ACAGTGCGCAAAGTATTGTCCATGCCCCAAGTCAATATTGTCAAG
AAAACAGAAGTACAGACAGGCGGATTCTCCAAGGAGTCAATTTTA
CCAAAAAGAAATTCGGACAAGCTTATTGCTCGTAAAAAAGACTGG
GATCCAAAAAAATATGGTGGTTTTGATAGTCCAACGGTAGCTTAT
TCAGTCCTAGTGGTTGCTAAGGTGGAAAAAGGGAAATCGAAGAAG
TTAAAATCCGTTAAAGAGTTACTAGGGATCACAATTATGGAAAGA
AGTTCCTTTGAAAAAAATCCGATTGACTTTTTAGAAGCTAAAGGA
TATAAGGAAGTTAAAAAAGACTTAATCATTAAACTACCTAAATAT
AGTCTTTTTGAGTTAGAAAACGGTCGTAAACGGATGCTGGCTAGT
GCCGGAGAATTACAAAAAGGAAATGAGCTGGCTCTGCCAAGCAAA
TATGTGAATTTTTTATATTTAGCTAGTCATTATGAAAAGTTGAAG
GGTAGTCCAGAAGATAACGAACAAAAACAATTGTTTGTGGAGCAG
CATAAGCATTATTTAGATGAGATTATTGAGCAAATCAGTGAATTT
TCTAAGCGTGTTATTTTAGCAGATGCCAATTTAGATAAAGTTCTT
AGTGCATATAACAAACATAGAGACAAACCAATACGTGAACAAGCA
GAAAATATTATTCATTTATTTACGTTGACGAATCTTGGAGCTCCC
GCTGCTTTTAAATATTTTGATACAACAATTGATCGTAAACGATAT
ACGTCTACAAAAGAAGTTTTAGATGCCACTCTTATCCATCAATCC
ATCACTGGTCTTTATGAAACACGCATTGATTTGAGTCAGCTAGGA
GGTGACTGA
(SEQ ID NO: 2)
MDKKYSIGLDIGTNSVGWAVITDDYKVPSKKFKVLGNTDRHSIKK
NLIGALLFGSGETAEATRLKRTARRRYTRRKNRICYLQEIFSNEM
AKVDDSFFHRLEESFLVEEDKKHERHPIFGNIVDEVAYHEKYPTI
YHLRKKLADSTDKADLRLIYLALAHMIKFRGHFLIEGDLNPDNSD
VDKLFIQLVQIYNQLFEENPINASRVDAKAILSARLSKSRRLENL
IAQLPGEKRNGLFGNLIALSLGLTPNFKSNFDLAEDAKLQLSKDT
YDDDLDNLLAQIGDQYADLFLAAKNLSDAILLSDILRVNSEITKA
PLSASMIKRYDEHHQDLTLLKALVRQQLPEKYKEIFFDQSKNGYA
GYIDGGASQEEFYKFIKPILEKMDGTEELLVKLNREDLLRKQRTF
DNGSIPHQIHLGELHAILRRQEDFYPFLKDNREKIEKILTFRIPY
YVGPLARGNSRFAWMTRKSEETITPWNFEEVVDKGASAQSFIERM
TNFDKNLPNEKVLPKHSLLYEYFTVYNELTKVKYVTEGMRKPAFL
SGEQKKAIVDLLFKTNRKVTVKQLKEDYFKKIECFDSVEISGVED
RFNASLGAYHDLLKIIKDKDFLDNEENEDILEDIVLTLTLFEDRG
MIEERLKTYAHLFDDKVMKQLKRRRYTGWGRLSRKLINGIRDKQS
GKTILDFLKSDGFANRNFMQLIHDDSLTFKEDIQKAQVSGQGHSL
HEQIANLAGSPAIKKGILQTVKIVDELVKVMGHKPENIVIEMARE
NQTTQKGQKNSRERMKRIEEGIKELGSQILKEHPVENTQLQNEKL
YLYYLQNGRDMYVDQELDINRLSDYDVDHIVPQSFIKDDSIDNKV
LTRSDKNRGKSDNVPSEEVVKKMKNYWRQLLNAKLITQRKFDNLT
KAERGGLSELDKAGFIKRQLVETRQITKHVAQILDSRMNTKYDEN
DKLIREVKVITLKSKLVSDERKDFQFYKVREINNYHHAHDAYLNA
VVGTALIKKYPKLESEFVYGDYKVYDVRKMIAKSEQEIGKATAKY
FFYSNIMNFFKTEITLANGEIRKRPLIETNGETGEIVWDKGRDFA
TVRKVLSMPQVNIVKKTEVQTGGFSKESILPKRNSDKLIARKKDW
DPKKYGGFDSPTVAYSVLVVAKVEKGKSKKLKSVKELLGITIMER
SSFEKNPIDFLEAKGYKEVKKDLIIKLPKYSLFELENGRKRMLAS
AGELQKGNELALPSKYVNFLYLASHYEKLKGSPEDNEQKQLFVEQ
HKHYLDEIIEQISEFSKRVILADANLDKVLSAYNKHRDKPIREQA
ENIIHLFTLTNLGAPAAFKYFDTTIDRKRYTSTKEVLDATLIHQS
ITGLYETRIDLSQLGGD
(single underline: HNH domain;
double underline: RuvC domain)

In some embodiments, wild type Cas9 corresponds to, or comprises SEQ ID NO:3 (nucleotide) and/or SEQ ID NO: 4 (amino acid):

(SEQ ID NO: 3)
ATGGATAAAAAGTATTCTATTGGTTTAGACATCGGCACTAATTCC
GTTGGATGGGCTGTCATAACCGATGAATACAAAGTACCTTCAAAG
AAATTTAAGGTGTTGGGGAACACAGACCGTCATTCGATTAAAAAG
AATCTTATCGGTGCCCTCCTATTCGATAGTGGCGAAACGGCAGAG
GCGACTCGCCTGAAACGAACCGCTCGGAGAAGGTATACACGTCGC
AAGAACCGAATATGTTACTTACAAGAAATTTTTAGCAATGAGATG
GCCAAAGTTGACGATTCTTTCTTTCACCGTTTGGAAGAGTCCTTC
CTTGTCGAAGAGGACAAGAAACATGAACGGCACCCCATCTTTGGA
AACATAGTAGATGAGGTGGCATATCATGAAAAGTACCCAACGATT
TATCACCTCAGAAAAAAGCTAGTTGACTCAACTGATAAAGCGGAC
CTGAGGTTAATCTACTTGGCTCTTGCCCATATGATAAAGTTCCGT
GGGCACTTTCTCATTGAGGGTGATCTAAATCCGGACAACTCGGAT
GTCGACAAACTGTTCATCCAGTTAGTACAAACCTATAATCAGTTG
TTTGAAGAGAACCCTATAAATGCAAGTGGCGTGGATGCGAAGGCT
ATTCTTAGCGCCCGCCTCTCTAAATCCCGACGGCTAGAAAACCTG
ATCGCACAATTACCCGGAGAGAAGAAAAATGGGTTGTTCGGTAAC
CTTATAGCGCTCTCACTAGGCCTGACACCAAATTTTAAGTCGAAC
TTCGACTTAGCTGAAGATGCCAAATTGCAGCTTAGTAAGGACACG
TACGATGACGATCTCGACAATCTACTGGCACAAATTGGAGATCAG
TATGCGGACTTATTTTTGGCTGCCAAAAACCTTAGCGATGCAATC
CTCCTATCTGACATACTGAGAGTTAATACTGAGATTACCAAGGCG
CCGTTATCCGCTTCAATGATCAAAAGGTACGATGAACATCACCAA
GACTTGACACTTCTCAAGGCCCTAGTCCGTCAGCAACTGCCTGAG
AAATATAAGGAAATATTCTTTGATCAGTCGAAAAACGGGTACGCA
GGTTATATTGACGGCGGAGCGAGTCAAGAGGAATTCTACAAGTTT
ATCAAACCCATATTAGAGAAGATGGATGGGACGGAAGAGTTGCTT
GTAAAACTCAATCGCGAAGATCTACTGCGAAAGCAGCGGACTTTC
GACAACGGTAGCATTCCACATCAAATCCACTTAGGCGAATTGCAT
GCTATACTTAGAAGGCAGGAGGATTTTTATCCGTTCCTCAAAGAC
AATCGTGAAAAGATTGAGAAAATCCTAACCTTTCGCATACCTTAC
TATGTGGGACCCCTGGCCCGAGGGAACTCTCGGTTCGCATGGATG
ACAAGAAAGTCCGAAGAAACGATTACTCCATGGAATTTTGAGGAA
GTTGTCGATAAAGGTGCGTCAGCTCAATCGTTCATCGAGAGGATG
ACCAACTTTGACAAGAATTTACCGAACGAAAAAGTATTGCCTAAG
CACAGTTTACTTTACGAGTATTTCACAGTGTACAATGAACTCACG
AAAGTTAAGTATGTCACTGAGGGCATGCGTAAACCCGCCTTTCTA
AGCGGAGAACAGAAGAAAGCAATAGTAGATCTGTTATTCAAGACC
AACCGCAAAGTGACAGTTAAGCAATTGAAAGAGGACTACTTTAAG
AAAATTGAATGCTTCGATTCTGTCGAGATCTCCGGGGTAGAAGAT
CGATTTAATGCGTCACTTGGTACGTATCATGACCTCCTAAAGATA
ATTAAAGATAAGGACTTCCTGGATAACGAAGAGAATGAAGATATC
TTAGAAGATATAGTGTTGACTCTTACCCTCTTTGAAGATCGGGAA
ATGATTGAGGAAAGACTAAAAACATACGCTCACCTGTTCGACGAT
AAGGTTATGAAACAGTTAAAGAGGCGTCGCTATACGGGCTGGGGA
CGATTGTCGCGGAAACTTATCAACGGGATAAGAGACAAGCAAAGT
GGTAAAACTATTCTCGATTTTCTAAAGAGCGACGGCTTCGCCAAT
AGGAACTTTATGCAGCTGATCCATGATGACTCTTTAACCTTCAAA
GAGGATATACAAAAGGCACAGGTTTCCGGACAAGGGGACTCATTG
CACGAACATATTGCGAATCTTGCTGGTTCGCCAGCCATCAAAAAG
GGCATACTCCAGACAGTCAAAGTAGTGGATGAGCTAGTTAAGGTC
ATGGGACGTCACAAACCGGAAAACATTGTAATCGAGATGGCACGC
GAAAATCAAACGACTCAGAAGGGGCAAAAAAACAGTCGAGAGCGG
ATGAAGAGAATAGAAGAGGGTATTAAAGAACTGGGCAGCCAGATC
TTAAAGGAGCATCCTGTGGAAAATACCCAATTGCAGAACGAGAAA
CTTTACCTCTATTACCTACAAAATGGAAGGGACATGTATGTTGAT
CAGGAACTGGACATAAACCGTTTATCTGATTACGACGTCGATCAC
ATTGTACCCCAATCCTTTTTGAAGGACGATTCAATCGACAATAAA
GTGCTTACACGCTCGGATAAGAACCGAGGGAAAAGTGACAATGTT
CCAAGCGAGGAAGTCGTAAAGAAAATGAAGAACTATTGGCGGCAG
CTCCTAAATGCGAAACTGATAACGCAAAGAAAGTTCGATAACTTA
ACTAAAGCTGAGAGGGGGGCTTGTCTGAACTTGACAAGGCCGGAT
TTATTAAACGTCAGCTCGTGGAAACCCGCCAAATCACAAAGCATG
TTGCACAGATACTAGATTCCCGAATGAATACGAAATACGACGAGA
ACGATAAGCTGATTCGGGAAGTCAAAGTAATCACTTTAAAGTCAA
AATTGGTGTCGGACTTCAGAAAGGATTTTCAATTCTATAAAGTTA
GGGAGATAAATAACTACCACCATGCGCACGACGCTTATCTTAATG
CCGTCGTAGGGACCGCACTCATTAAGAAATACCCGAAGCTAGAAA
GTGAGTTTGTGTATGGTGATTACAAAGTTTATGACGTCCGTAAGA
TGATCGCGAAAAGCGAACAGGAGATAGGCAAGGCTACAGCCAAAT
ACTTCTTTTATTCTAACATTATGAATTTCTTTAAGACGGAAATCA
CTCTGGCAAACGGAGAGATACGCAAACGACCTTTAATTGAAACCA
ATGGGGAGACAGGTGAAATCGTATGGGATAAGGGCCGGGACTTCG
CGACGGTGAGAAAAGTTTTGTCCATGCCCCAAGTCAACATAGTAA
AGAAAACTGAGGTGCAGACCGGAGGGTTTTCAAAGGAATCGATTC
TTCCAAAAAGGAATAGTGATAAGCTCATCGCTCGTAAAAAGGACT
GGGACCCGAAAAAGTACGGTGGCTTCGATAGCCCTACAGTTGCCT
ATTCTGTCCTAGTAGTGGCAAAAGTTGAGAAGGGAAAATCCAAGA
AACTGAAGTCAGTCAAAGAATTATTGGGGATAACGATTATGGAGC
GCTCGTCTTTTGAAAAGAACCCCATCGACTTCCTTGAGGCGAAAG
GTTACAAGGAAGTAAAAAAGGATCTCATAATTAAACTACCAAAGT
ATAGTCTGTTTGAGTTAGAAAATGGCCGAAAACGGATGTTGGCTA
GCGCCGGAGAGCTTCAAAAGGGGAACGAACTCGCACTACCGTCTA
AATACGTGAATTTCCTGTATTTAGCGTCCCATTACGAGAAGTTGA
AAGGTTCACCTGAAGATAACGAACAGAAGCAACTTTTTGTTGAGC
AGCACAAACATTATCTCGACGAAATCATAGAGCAAATTTCGGAAT
TCAGTAAGAGAGTCATCCTAGCTGATGCCAATCTGGACAAAGTAT
TAAGCGCATACAACAAGCACAGGGATAAACCCATACGTGAGCAGG
CGGAAAATATTATCCATTTGTTTACTCTTACCAACCTCGGCGCTC
CAGCCGCATTCAAGTATTTTGACACAACGATAGATCGCAAACGAT
ACACTTCTACCAAGGAGGTGCTAGACGCGACACTGATTCACCAAT
CCATCACGGGATTATATGAAACTCGGATAGATTTGTCACAGCTTG
GGGGTGACGGATCCCCCAAGAAGAAGAGGAAAGTCTCGAGCGACT
ACAAAGACCATGACGGTGATTATAAAGATCATGACATCGATTACA
AGGATGACGATGACAAGGCTGCAGGA
(SEQ ID NO: 4)
MDKKYSIGLAIGTNSVGWAVITDEYKVPSKKFKVLGNTDRHSIKK
NLIGALLFDSGETAEATRLKRTARRRYTRRKNRICYLQEIFSNEM
AKVDDSFFHRLEESFLVEEDKKHERHPIFGNIVDEVAYHEKYPTI
YHLRKKLVDSTDKADLRLIYLALAHMIKFRGHFLIEGDLNPDNSD
VDKLFIQLVQTYNQLFEENPINASGVDAKAILSARLSKSRRLENL
IAQLPGEKKNGLFGNLIALSLGLTPNFKSNFDLAEDAKLQLSKDT
YDDDLDNLLAQIGDQYADLFLAAKNLSDAILLSDILRVNTEITKA
PLSASMIKRYDEHHQDLTLLKALVRQQLPEKYKEIFFDQSKNGYA
GYIDGGASQEEFYKFIKPILEKMDGTEELLVKLNREDLLRKQRTF
DNGSIPHQIHLGELHAILRRQEDFYPFLKDNREKIEKILTFRIPY
YVGPLARGNSRFAWMTRKSEETITPWNFEEVVDKGASAQSFIERM
TNFDKNLPNEKVLPKHSLLYEYFTVYNELTKVKYVTEGMRKPAFL
SGEQKKAIVDLLFKTNRKVTVKQLKEDYFKKIECFDSVEISGVED
RFNASLGTYHDLLKIIKDKDFLDNEENEDILEDIVLTLTLFEDRE
MIEERLKTYAHLFDDKVMKQLKRRRYTGWGRLSRKLINGIRDKQS
GKTILDFLKSDGFANRNFMQLIHDDSLTFKEDIQKAQVSGQGDSL
HEHIANLAGSPAIKKGILQTVKVVDELVKVMGRHKPENIVIEMAR
ENQTTQKGQKNSRERMKRIEEGIKELGSQILKEHPVENTQLQNEK
LYLYYLQNGRDMYVDQELDINRLSDYDVDHIVPQSFLKDDSIDNK
VLTRSDKNRGKSDNVPSEEVVKKMKNYWRQLLNAKLITQRKFDNL
TKAERGGLSELDKAGFIKRQLVETRQITKHVAQILDSRMNTKYDE
NDKLIREVKVITLKSKLVSDERKDFQFYKVREINNYHHAHDAYLN
AVVGTALIKKYPKLESEFVYGDYKVYDVRKMIAKSEQEIGKATAK
YFFYSNIMNFFKTEITLANGEIRKRPLIETNGETGEIVWDKGRDF
ATVRKVLSMPQVNIVKKTEVQTGGFSKESILPKRNSDKLIARKKD
WDPKKYGGFDSPTVAYSVLVVAKVEKGKSKKLKSVKELLGITIME
RSSFEKNPIDFLEAKGYKEVKKDLIIKLPKYSLFELENGRKRMLA
SAGELQKGNELALPSKYVNFLYLASHYEKLKGSPEDNEQKQLFVE
QHKHYLDEIIEQISEFSKRVILADANLDKVLSAYNKHRDKPIREQ
AENIIHLFTLTNLGAPAAFKYFDTTIDRKRYTSTKEVLDATLIHQ
SITGLYETRIDLSQLGGD
(single underline: HNH domain;
double underline: RuvC domain)

In some embodiments, wild type Cas9 corresponds to Cas9 from Streptococcus pyogenes (NCBI Reference Sequence: NC_002737.2, SEQ ID NO: 8 (nucleotide); and Uniport Reference Sequence: Q99ZW2, SEQ ID NO: 10 (amino acid).

(SEQ ID NO: 8)
ATGGATAAGAAATACTCAATAGGCTTAGATATCGGCACAAATAGC
GTCGGATGGGCGGTGATCACTGATGAATATAAGGTTCCGTCTAAA
AAGTTCAAGGTTCTGGGAAATACAGACCGCCACAGTATCAAAAAA
AATCTTATAGGGGCTCTTTTATTTGACAGTGGAGAGACAGCGGAA
GCGACTCGTCTCAAACGGACAGCTCGTAGAAGGTATACACGTCGG
AAGAATCGTATTTGTTATCTACAGGAGATTTTTTCAAATGAGATG
GCGAAAGTAGATGATAGTTTCTTTCATCGACTTGAAGAGTCTTTT
TTGGTGGAAGAAGACAAGAAGCATGAACGTCATCCTATTTTTGGA
AATATAGTAGATGAAGTTGCTTATCATGAGAAATATCCAACTATC
TATCATCTGCGAAAAAAATTGGTAGATTCTACTGATAAAGCGGAT
TTGCGCTTAATCTATTTGGCCTTAGCGCATATGATTAAGTTTCGT
GGTCATTTTTTGATTGAGGGAGATTTAAATCCTGATAATAGTGAT
GTGGACAAACTATTTATCCAGTTGGTACAAACCTACAATCAATTA
TTTGAAGAAAACCCTATTAACGCAAGTGGAGTAGATGCTAAAGCG
ATTCTTTCTGCACGATTGAGTAAATCAAGACGATTAGAAAATCTC
ATTGCTCAGCTCCCCGGTGAGAAGAAAAATGGCTTATTTGGGAAT
CTCATTGCTTTGTCATTGGGTTTGACCCCTAATTTTAAATCAAAT
TTTGATTTGGCAGAAGATGCTAAATTACAGCTTTCAAAAGATACT
TACGATGATGATTTAGATAATTTATTGGCGCAAATTGGAGATCAA
TATGCTGATTTGTTTTTGGCAGCTAAGAATTTATCAGATGCTATT
TTACTTTCAGATATCCTAAGAGTAAATACTGAAATAACTAAGGCT
CCCCTATCAGCTTCAATGATTAAACGCTACGATGAACATCATCAA
GACTTGACTCTTTTAAAAGCTTTAGTTCGACAACAACTTCCAGAA
AAGTATAAAGAAATCTTTTTTGATCAATCAAAAAACGGATATGCA
GGTTATATTGATGGGGGAGCTAGCCAAGAAGAATTTTATAAATTT
ATCAAACCAATTTTAGAAAAAATGGATGGTACTGAGGAATTATTG
GTGAAACTAAATCGTGAAGATTTGCTGCGCAAGCAACGGACCTTT
GACAACGGCTCTATTCCCCATCAAATTCACTTGGGTGAGCTGCAT
GCTATTTTGAGAAGACAAGAAGACTTTTATCCATTTTTAAAAGAC
AATCGTGAGAAGATTGAAAAAATCTTGACTTTTCGAATTCCTTAT
TATGTTGGTCCATTGGCGCGTGGCAATAGTCGTTTTGCATGGATG
ACTCGGAAGTCTGAAGAAACAATTACCCCATGGAATTTTGAAGAA
GTTGTCGATAAAGGTGCTTCAGCTCAATCATTTATTGAACGCATG
ACAAACTTTGATAAAAATCTTCCAAATGAAAAAGTACTACCAAAA
CATAGTTTGCTTTATGAGTATTTTACGGTTTATAACGAATTGACA
AAGGTCAAATATGTTACTGAAGGAATGCGAAAACCAGCATTTCTT
TCAGGTGAACAGAAGAAAGCCATTGTTGATTTACTCTTCAAAACA
AATCGAAAAGTAACCGTTAAGCAATTAAAAGAAGATTATTTCAAA
AAAATAGAATGTTTTGATAGTGTTGAAATTTCAGGAGTTGAAGAT
AGATTTAATGCTTCATTAGGTACCTACCATGATTTGCTAAAAATT
ATTAAAGATAAAGATTTTTTGGATAATGAAGAAAATGAAGATATC
TTAGAGGATATTGTTTTAACATTGACCTTATTTGAAGATAGGGAG
ATGATTGAGGAAAGACTTAAAACATATGCTCACCTCTTTGATGAT
AAGGTGATGAAACAGCTTAAACGTCGCCGTTATACTGGTTGGGGA
CGTTTGTCTCGAAAATTGATTAATGGTATTAGGGATAAGCAATCT
GGCAAAACAATATTAGATTTTTTGAAATCAGATGGTTTTGCCAAT
CGCAATTTTATGCAGCTGATCCATGATGATAGTTTGACATTTAAA
GAAGACATTCAAAAAGCACAAGTGTCTGGACAAGGCGATAGTTTA
CATGAACATATTGCAAATTTAGCTGGTAGCCCTGCTATTAAAAAA
GGTATTTTACAGACTGTAAAAGTTGTTGATGAATTGGTCAAAGTA
ATGGGGCGGCATAAGCCAGAAAATATCGTTATTGAAATGGCACGT
GAAAATCAGACAACTCAAAAGGGCCAGAAAAATTCGCGAGAGCGT
ATGAAACGAATCGAAGAAGGTATCAAAGAATTAGGAAGTCAGATT
CTTAAAGAGCATCCTGTTGAAAATACTCAATTGCAAAATGAAAAG
CTCTATCTCTATTATCTCCAAAATGGAAGAGACATGTATGTGGAC
CAAGAATTAGATATTAATCGTTTAAGTGATTATGATGTCGATCAC
ATTGTTCCACAAAGTTTCCTTAAAGACGATTCAATAGACAATAAG
GTCTTAACGCGTTCTGATAAAAATCGTGGTAAATCGGATAACGTT
CCAAGTGAAGAAGTAGTCAAAAAGATGAAAAACTATTGGAGACAA
CTTCTAAACGCCAAGTTAATCACTCAACGTAAGTTTGATAATTTA
ACGAAAGCTGAACGTGGAGGTTTGAGTGAACTTGATAAAGCTGGT
TTTATCAAACGCCAATTGGTTGAAACTCGCCAAATCACTAAGCAT
GTGGCACAAATTTTGGATAGTCGCATGAATACTAAATACGATGAA
AATGATAAACTTATTCGAGAGGTTAAAGTGATTACCTTAAAATCT
AAATTAGTTTCTGACTTCCGAAAAGATTTCCAATTCTATAAAGTA
CGTGAGATTAACAATTACCATCATGCCCATGATGCGTATCTAAAT
GCCGTCGTTGGAACTGCTTTGATTAAGAAATATCCAAAACTTGAA
TCGGAGTTTGTCTATGGTGATTATAAAGTTTATGATGTTCGTAAA
ATGATTGCTAAGTCTGAGCAAGAAATAGGCAAAGCAACCGCAAAA
TATTTCTTTTACTCTAATATCATGAACTTCTTCAAAACAGAAATT
ACACTTGCAAATGGAGAGATTCGCAAACGCCCTCTAATCGAAACT
AATGGGGAAACTGGAGAAATTGTCTGGGATAAAGGGCGAGATTTT
GCCACAGTGCGCAAAGTATTGTCCATGCCCCAAGTCAATATTGTC
AAGAAAACAGAAGTACAGACAGGCGGATTCTCCAAGGAGTCAATT
TTACCAAAAAGAAATTCGGACAAGCTTATTGCTCGTAAAAAAGAC
TGGGATCCAAAAAAATATGGTGGTTTTGATAGTCCAACGGTAGCT
TATTCAGTCCTAGTGGTTGCTAAGGTGGAAAAAGGGAAATCGAAG
AAGTTAAAATCCGTTAAAGAGTTACTAGGGATCACAATTATGGAA
AGAAGTTCCTTTGAAAAAAATCCGATTGACTTTTTAGAAGCTAAA
GGATATAAGGAAGTTAAAAAAGACTTAATCATTAAACTACCTAAA
TATAGTCTTTTTGAGTTAGAAAACGGTCGTAAACGGATGCTGGCT
AGTGCCGGAGAATTACAAAAAGGAAATGAGCTGGCTCTGCCAAGC
AAATATGTGAATTTTTTATATTTAGCTAGTCATTATGAAAAGTTG
AAGGGTAGTCCAGAAGATAACGAACAAAAACAATTGTTTGTGGAG
CAGCATAAGCATTATTTAGATGAGATTATTGAGCAAATCAGTGAA
TTTTCTAAGCGTGTTATTTTAGCAGATGCCAATTTAGATAAAGTT
CTTAGTGCATATAACAAACATAGAGACAAACCAATACGTGAACAA
GCAGAAAATATTATTCATTTATTTACGTTGACGAATCTTGGAGCT
CCCGCTGCTTTTAAATATTTTGATACAACAATTGATCGTAAACGA
TATACGTCTACAAAAGAAGTTTTAGATGCCACTCTTATCCATCAA
TCCATCACTGGTCTTTATGAAACACGCATTGATTTGAGTCAGCTA
GGAGGTGACTGA
(SEQ ID NO: 10)
MDKKYSIGLDIGTNSVGWAVITDEYKVPSKKFKVLGNTDRHSIKK
NLIGALLEDSGETAEATRLKRTARRRYTRRKNRICYLQEIFSNEM
AKVDDSFFHRLEESFLVEEDKKHERHPIFGNIVDEVAYHEKYPTI
YHLRKKLVDSTDKADLRLIYLALAHMIKFRGHFLIEGDLNPDNSD
VDKLFIQLVQTYNQLFEENPINASGVDAKAILSARLSKSRRLENL
IAQLPGEKKNGLFGNLIALSLGLTPNFKSNFDLAEDAKLQLSKDT
YDDDLDNLLAQIGDQYADLFLAAKNLSDAILLSDILRVNTEITKA
PLSASMIKRYDEHHQDLTLLKALVRQQLPEKYKEIFFDQSKNGYA
GYIDGGASQEEFYKFIKPILEKMDGTEELLVKLNREDLLRKQRTF
DNGSIPHQIHLGELHAILRRQEDFYPFLKDNREKIEKILTFRIPY
YVGPLARGNSRFAWMTRKSEETITPWNFEEVVDKGASAQSFIERM
TNFDKNLPNEKVLPKHSLLYEYFTVYNELTKVKYVTEGMRKPAFL
SGEQKKAIVDLLFKTNRKVTVKQLKEDYFKKIECFDSVEISGVED
RFNASLGTYHDLLKIIKDKDFLDNEENEDILEDIVLTLTLFEDRE
MIEERLKTYAHLFDDKVMKQLKRRRYTGWGRLSRKLINGIRDKQS
GKTILDFLKSDGFANRNFMQLIHDDSLTFKEDIQKAQVSGQGDSL
HEHIANLAGSPAIKKGILQTVKVVDELVKVMGRHKPENIVIEMAR
ENQTTQKGQKNSRERMKRIEEGIKELGSQILKEHPVENTQLQNEK
LYLYYLQNGRDMYVDQELDINRLSDYDVDHIVPQSFLKDDSIDNK
VLTRSDKNRGKSDNVPSEEVVKKMKNYWRQLLNAKLITQRKFDNL
TKAERGGLSELDKAGFIKRQLVETRQITKHVAQILDSRMNTKYDE
NDKLIREVKVITLKSKLVSDERKDFQFYKVREINNYHHAHDAYLN
AVVGTALIKKYPKLESEFVYGDYKVYDVRKMIAKSEQEIGKATAK
YFFYSNIMNFFKTEITLANGEIRKRPLIETNGETGEIVWDKGRDF
ATVRKVLSMPQVNIVKKTEVQTGGFSKESILPKRNSDKLIARKKD
WDPKKYGGFDSPTVAYSVLVVAKVEKGKSKKLKSVKELLGITIME
RSSFEKNPIDFLEAKGYKEVKKDLIIKLPKYSLFELENGRKRMLA
SAGELQKGNELALPSKYVNFLYLASHYEKLKGSPEDNEQKQLFVE
QHKHYLDEIIEQISEFSKRVILADANLDKVLSAYNKHRDKPIREQ
AENIIHLFTLTNLGAPAAFKYFDTTIDRKRYTSTKEVLDATLIHQ
SITGLYETRIDLSQLGGD
(single underline: HNH domain;
double underline: RuvC domain)

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI (NCBI Ref: NC_018721.1); Streptococcus thermophilus (NCBI Ref: YP_820832.1), Listeria innocua (NCBI Ref: NP_472073.1), Campylobacter jejuni (NCBI Ref: YP_002344900.1) or Neisseria. meningitidis (NCBI Ref: YP_002342100.1) or to a Cas9 from any of the organisms listed in Example 5.

In some embodiments, dCas9 corresponds to, or comprises in part or in whole, a Cas9 amino acid sequence having one or more mutations that inactivate the Cas9 nuclease activity. For example, in some embodiments, a dCas9 domain comprises D10A and/or H840A mutation.

dCas9 (D10A and H840A):

(SEQ ID NO: 9)
MDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHERHPI
FGNIVDEVAYHEKYPTIYHLRKKLVDSTDKADLRLIYLALAHMIKFRGHFLIEGDLNPDN
SDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWG

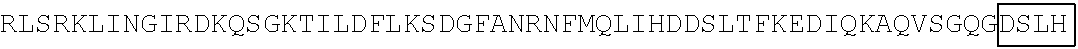

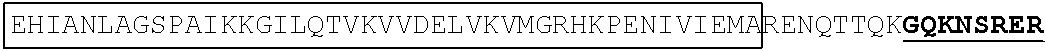

MKRIEEGIKELGSQILKEHPVENTQLQNEKLYLYYLONGRDMYVDQELDINRLSDY
DVDAIVPQSFLKDDSIDNKVLTRSDKNRGKSDNVPSEEVVKKMKNYWROLLNAKLI

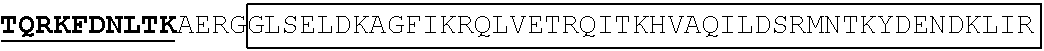

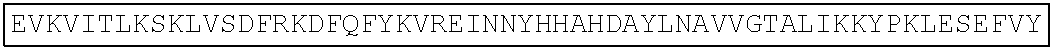

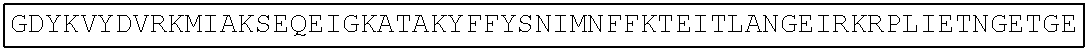

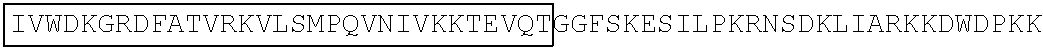

YGGFDSPTVAYSVLVVAKVEKGKSKKLKSVKELLGITIMERSSFEKNPIDFLEAKGYKEV
KKDLIIKLPKYSLFELENGRKRMLASAGELQKGNELALPSKYVNFLYLASHYEKLKGSPE
DNEQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGD
(single underline: HNH domain; double underline: RuvC domain).

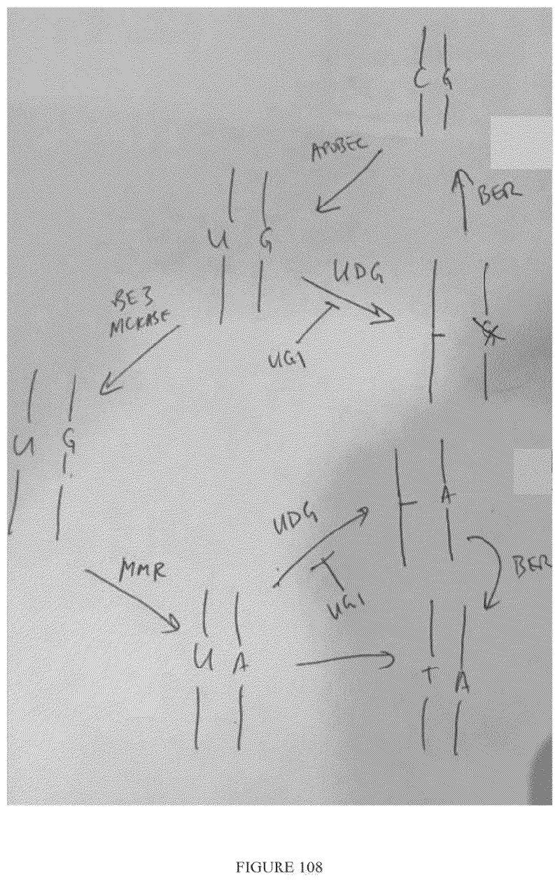

In some embodiments, the Cas9 domain comprises a D10A mutation, while the residue at position 840 remains a histidine in the amino acid sequence provided in SEQ ID NO: 10, or at corresponding positions in any of the amino acid sequences provided in SEQ ID NOs: 11-260. Without wishing to be bound by any particular theory, the presence of the catalytic residue H840 restores the activity of the Cas9 to cleave the non-edited (e.g., non-deaminated) strand containing a G opposite the targeted C. Restoration of H840 (e.g., from A840) does not result in the cleavage of the target strand containing the C. Such Cas9 variants are able to generate a single-strand DNA break (nick) at a specific location based on the gRNA-defined target sequence, leading to repair of the non-edited strand, ultimately resulting in a G to A change on the non-edited strand. A schematic representation of this process is shown in FIG. 108. Briefly, the C of a C-G basepair can be deaminated to a U by a deaminase, e.g., an APOBEC deamonase. Nicking the non-edited strand, having the G, facilitates removal of the G via mismatch repair mechanisms. UGI inhibits UDG, which prevents removal of the U.

In other embodiments, dCas9 variants having mutations other than D10A and H840A are provided, which, e.g., result in nuclease inactivated Cas9 (dCas9). Such mutations, by way of example, include other amino acid substitutions at D10 and H820, or other substitutions within the nuclease domains of Cas9 (e.g., substitutions in the HNH nuclease subdomain and/or the RuvC1 subdomain). In some embodiments, variants or homologues of dCas9 (e.g., variants of SEQ ID NO: 10) are provided which are at least about 70% identical, at least about 80% identical, at least about 90% identical, at least about 95% identical, at least about 98% identical, at least about 99% identical, at least about 99.5% identical, or at least about 99.9% identical to SEQ ID NO: 10. In some embodiments, variants of dCas9 (e.g., variants of SEQ ID NO: 10) are provided having amino acid sequences which are shorter, or longer than SEQ ID NO: 10, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

In some embodiments, Cas9 fusion proteins as provided herein comprise the full-length amino acid sequence of a Cas9 protein, e.g., one of the Cas9 sequences provided herein. In other embodiments, however, fusion proteins as provided herein do not comprise a full-length Cas9 sequence, but only a fragment thereof. For example, in some embodiments, a Cas9 fusion protein provided herein comprises a Cas9 fragment, wherein the fragment binds crRNA and tracrRNA or sgRNA, but does not comprise a functional nuclease domain, e.g., in that it comprises only a truncated version of a nuclease domain or no nuclease domain at all. Exemplary amino acid sequences of suitable Cas9 domains and Cas9 fragments are provided herein, and additional suitable sequences of Cas9 domains and fragments will be apparent to those of skill in the art.

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria. meningitidis (NCBI Ref: YP_002342100.1).

The term “deaminase” or “deaminase domain,” as used herein, refers to a protein or enzyme that catalyzes a deamination reaction. In some embodiments, the deaminase or deaminase domain is a cytidine deaminase, catalyzing the hydrolytic deamination of cytidine or deoxycytidine to uridine or deoxyuridine, respectively. In some embodiments, the deaminase or deaminase domain is a cytidine deaminase domain, catalyzing the hydrolytic deamination of cytosine to uracil. In some embodiments, the deaminase or deaminase domain is a naturally-occurring deaminase from an organism, such as a human, chimpanzee, gorilla, monkey, cow, dog, rat, or mouse. In some embodiments, the deaminase or deaminase domain is a variant of a naturally-occurring deaminase from an organism, that does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism.

The term “effective amount,” as used herein, refers to an amount of a biologically active agent that is sufficient to elicit a desired biological response. For example, in some embodiments, an effective amount of a nuclease may refer to the amount of the nuclease that is sufficient to induce cleavage of a target site specifically bound and cleaved by the nuclease. In some embodiments, an effective amount of a fusion protein provided herein, e.g., of a fusion protein comprising a nuclease-inactive Cas9 domain and a nucleic acid editing domain (e.g., a deaminase domain) may refer to the amount of the fusion protein that is sufficient to induce editing of a target site specifically bound and edited by the fusion protein. As will be appreciated by the skilled artisan, the effective amount of an agent, e.g., a fusion protein, a nuclease, a deaminase, a recombinase, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the cell or tissue being targeted, and on the agent being used.

The term “linker,” as used herein, refers to a chemical group or a molecule linking two molecules or moieties, e.g., two domains of a fusion protein, such as, for example, a nuclease-inactive Cas9 domain and a nucleic acid editing domain (e.g., a deaminase domain). In some embodiments, a linker joins a gRNA binding domain of an RNA-programmable nuclease, including a Cas9 nuclease domain, and the catalytic domain of a nucleic-acid editing protein. In some embodiments, a linker joins a dCas9 and a nucleic-acid editing protein. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “nucleic acid editing domain,” as used herein refers to a protein or enzyme capable of making one or more modifications (e.g., deamination of a cytidine residue) to a nucleic acid (e.g., DNA or RNA). Exemplary nucleic acid editing domains include, but are not limited to a deaminase, a nuclease, a nickase, a recombinase, a methyltransferase, a methylase, an acetylase, an acetyltransferase, a transcriptional activator, or a transcriptional repressor domain. In some embodiments the nucleic acid editing domain is a deaminase (e.g., a cytidine deaminase, such as an APOBEC or an AID deaminase).

The term “proliferative disease,” as used herein, refers to any disease in which cell or tissue homeostasis is disturbed in that a cell or cell population exhibits an abnormally elevated proliferation rate. Proliferative diseases include hyperproliferative diseases, such as pre-neoplastic hyperplastic conditions and neoplastic diseases. Neoplastic diseases are characterized by an abnormal proliferation of cells and include both benign and malignant neoplasias. Malignant neoplasia is also referred to as cancer.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

The term “RNA-programmable nuclease,” and “RNA-guided nuclease” are used interchangeably herein and refer to a nuclease that forms a complex with (e.g., binds or associates with) one or more RNA that is not a target for cleavage. In some embodiments, an RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 complex to the target); and (2) a domain that binds a Cas9 protein. In some embodiments, domain (2) corresponds to a sequence known as a tracrRNA, and comprises a stem-loop structure. For example, in some embodiments, domain (2) is identical or homologous to a tracrRNA as provided in Jinek et al., Science 337:816-821(2012), the entire contents of which is incorporated herein by reference. Other examples of gRNAs (e.g., those including domain 2) can be found in U.S. Provisional Patent Application Ser. No. 61/874,682, filed Sep. 6, 2013, entitled “Switchable Cas9 Nucleases And Uses Thereof,” and U.S. Provisional Patent Application Ser. No. 61/874,746, filed Sep. 6, 2013, entitled “Delivery System For Functional Nucleases,” the entire contents of each are hereby incorporated by reference in their entirety. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example Cas9 (Csn1) from Streptococcus pyogenes (see, e.g., “Complete genome sequence of an M1 strain of Streptococcus pyogenes.” Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference.

Because RNA-programmable nucleases (e.g., Cas9) use RNA:DNA hybridization to target DNA cleavage sites, these proteins are able to be targeted, in principle, to any sequence specified by the guide RNA. Methods of using RNA-programmable nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature biotechnology 31, 227-229 (2013); Jinek, M. et al. RNA-programmed genome editing in human cells. eLife 2, e00471 (2013); Dicarlo, J. E. et al. Genome engineering in Saccharomyces cerevisiae using CRISPR-Cas systems. Nucleic acids research (2013); Jiang, W. et al. RNA-guided editing of bacterial genomes using CRISPR-Cas systems. Nature biotechnology 31, 233-239 (2013); the entire contents of each of which are incorporated herein by reference).

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

The term “target site” refers to a sequence within a nucleic acid molecule that is deaminated by a deaminase or a fusion protein comprising a deaminase, (e.g., a dCas9-deaminase fusion protein provided herein).

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

The term “nucleobase editors (NBEs)” or “base editors (BEs),” as used herein, refers to the Cas9 fusion proteins described herein. In some embodiments, the fusion protein comprises a nuclease-inactive Cas9 (dCas9) fused to a deaminase. In some embodiments, the fusion protein comprises a Cas9 nickase fused to a deaminase. In some embodiments, the fusion protein comprises a nuclease-inactive Cas9 fused to a deaminase and further fused to a UGI domain. In some embodiments, the fusion protein comprises a Cas9 nickase fused to a deaminase and further fused to a UGI domain. In some embodiments, the dCas9 of the fusion protein comprises a D10A and a H840A mutation of SEQ ID NO: 10, or a corresponding mutation in any of SEQ ID NOs: 11-260, which inactivates nuclease activity of the Cas9 protein. In some embodiments, the fusion protein comprises a D10A mutation and comprises a histidine at residue 840 of SEQ ID NO: 10, or a corresponding mutation in any of SEQ ID NOs: 11-260, which renders Cas9 capable of cleaving only one strand of a nucleic acid duplex. An example of a Cas9 nickase is shown below in SEQ ID NO: 674. The terms “nucleobase editors (NBEs)” and “base editors (BEs)” may be used interchangeably.

The term “uracil glycosylase inhibitor” or “UGI,” as used herein, refers to a protein that is capable of inhibiting a uracil-DNA glycosylase base-excision repair enzyme.

The term “Cas9 nickase,” as used herein, refers to a Cas9 protein that is capable of cleaving only one strand of a duplexed nucleic acid molecule (e.g., a duplexed DNA molecule). In some embodiments, a Cas9 nickase comprises a D10A mutation and has a histidine at position H840 of SEQ ID NO: 10, or a corresponding mutation in any of SEQ ID NOs: 11-260. For example, a Cas9 nickase may comprise the amino acid sequence as set forth in SEQ ID NO: 674. Such a Cas9 nickase has an active HNH nuclease domain and is able to cleave the non-targeted strand of DNA, i.e., the strand bound by the gRNA. Further, such a Cas9 nickase has an inactive RuvC nuclease domain and is not able to cleave the targeted strand of the DNA, i.e., the strand where base editing is desired.

Exemplary Cas9 nickase (Cloning vector pPlatTET-gRNA2; Accession No. BAV54124).

(SEQ ID NO: 674)
MDKKYSIGLAIGTNSVGWAVITDEYKVPSKKFKVLGNTDRHSIKK
NLIGALLFDSGETAEATRLKRTARRRYTRRKNRICYLQEIFSNEM
AKVDDSFFHRLEESFLVEEDKKHERHPIFGNIVDEVAYHEKYPTI
YHLRKKLVDSTDKADLRLIYLALAHMIKFRGHFLIEGDLNPDNSD
VDKLFIQLVQTYNQLFEENPINASGVDAKAILSARLSKSRRLENL
IAQLPGEKKNGLFGNLIALSLGLTPNFKSNFDLAEDAKLQLSKDT
YDDDLDNLLAQIGDQYADLFLAAKNLSDAILLSDILRVNTEITKA
PLSASMIKRYDEHHQDLTLLKALVRQQLPEKYKEIFFDQSKNGYA
GYIDGGASQEEFYKFIKPILEKMDGTEELLVKLNREDLLRKQRTF
DNGSIPHQIHLGELHAILRRQEDFYPFLKDNREKIEKILTFRIPY
YVGPLARGNSRFAWMTRKSEETITPWNFEEVVDKGASAQSFIERM
TNFDKNLPNEKVLPKHSLLYEYFTVYNELTKVKYVTEGMRKPAFL
SGEQKKAIVDLLFKTNRKVTVKQLKEDYFKKIECFDSVEISGVED
RFNASLGTYHDLLKIIKDKDFLDNEENEDILEDIVLTLTLFEDRE
MIEERLKTYAHLFDDKVMKQLKRRRYTGWGRLSRKLINGIRDKQS
GKTILDFLKSDGFANRNFMQLIHDDSLTFKEDIQKAQVSGQGDSL
HEHIANLAGSPAIKKGILQTVKVVDELVKVMGRHKPENIVIEMAR
ENQTTQKGQKNSRERMKRIEEGIKELGSQILKEHPVENTQLQNEK
LYLYYLQNGRDMYVDQELDINRLSDYDVDHIVPQSFLKDDSIDNK
VLTRSDKNRGKSDNVPSEEVVKKMKNYWRQLLNAKLITQRKFDNL
TKAERGGLSELDKAGFIKRQLVETRQITKHVAQILDSRMNTKYDE
NDKLIREVKVITLKSKLVSDFRKDFQFYKVREINNYHHAHDAYLN
AVVGTALIKKYPKLESEFVYGDYKVYDVRKMIAKSEQEIGKATAK
YFFYSNIMNFFKTEITLANGEIRKRPLIETNGETGEIVWDKGRDF
ATVRKVLSMPQVNIVKKTEVQTGGFSKESILPKRNSDKLIARKKD
WDPKKYGGFDSPTVAYSVLVVAKVEKGKSKKLKSVKELLGITIME
RSSFEKNPIDFLEAKGYKEVKKDLIIKLPKYSLFELENGRKRMLA
SAGELQKGNELALPSKYVNFLYLASHYEKLKGSPEDNEQKQLFVE
QHKHYLDEIIEQISEFSKRVILADANLDKVLSAYNKHRDKPIREQ
AENIIHLFTLTNLGAPAAFKYFDTTIDRKRYTSTKEVLDATLIHQ
SITGLYETRIDLSQLGGD

### DETAILED DESCRIPTION OF CERTAIN EMBODIMENTS OF THE INVENTION

Some aspects of this disclosure provide fusion proteins that comprise a domain capable of binding to a nucleotide sequence (e.g., a Cas9, or a Cpf1 protein) and an enzyme domain, for example, a DNA-editing domain, such as, e.g., a deaminase domain. The deamination of a nucleobase by a deaminase can lead to a point mutation at the respective residue, which is referred to herein as nucleic acid editing. Fusion proteins comprising a Cas9 variant or domain and a DNA editing domain can thus be used for the targeted editing of nucleic acid sequences. Such fusion proteins are useful for targeted editing of DNA in vitro, e.g., for the generation of mutant cells or animals; for the introduction of targeted mutations, e.g., for the correction of genetic defects in cells ex vivo, e.g., in cells obtained from a subject that are subsequently re-introduced into the same or another subject; and for the introduction of targeted mutations, e.g., the correction of genetic defects or the introduction of deactivating mutations in disease-associated genes in a subject. Typically, the Cas9 domain of the fusion proteins described herein does not have any nuclease activity but instead is a Cas9 fragment or a dCas9 protein or domain. Methods for the use of Cas9 fusion proteins as described herein are also provided.

### Cas9 Domains of Nucleobase Editors

Non-limiting, exemplary Cas9 domains are provided herein. The Cas9 domain may be a nuclease active Cas9 domain, a nuclease inactive Cas9 domain, or a Cas9 nickase. In some embodiments, the Cas9 domain is a nuclease active domain. For example, the Cas9 domain may be a Cas9 domain that cuts both strands of a duplexed nucleic acid (e.g., both strands of a duplexed DNA molecule). In some embodiments, the Cas9 domain comprises any one of the amino acid sequences as set forth in SEQ ID NOs: 10-263. In some embodiments the Cas9 domain comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in SEQ ID NOs: 10-263. In some embodiments, the Cas9 domain comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more or more mutations compared to any one of the amino acid sequences set forth in SEQ ID NOs: 10-263. In some embodiments, the Cas9 domain comprises an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences set forth in SEQ ID NOs: 10-263.

In some embodiments, the Cas9 domain is a nuclease-inactive Cas9 domain (dCas9). For example, the dCas9 domain may bind to a duplexed nucleic acid molecule (e.g., via a gRNA molecule) without cleaving either strand of the duplexed nucleic acid molecule. In some embodiments, the nuclease-inactive dCas9 domain comprises a D10X mutation and a H840X mutation of the amino acid sequence set forth in SEQ ID NO: 10, or a corresponding mutation in any of the amino acid sequences provided in SEQ ID NOs: 11-260, wherein X is any amino acid change. In some embodiments, the nuclease-inactive dCas9 domain comprises a D10A mutation and a H840A mutation of the amino acid sequence set forth in SEQ ID NO: 10, or a corresponding mutation in any of the amino acid sequences provided in SEQ ID NOs: 11-260. As one example, a nuclease-inactive Cas9 domain comprises the amino acid sequence set forth in SEQ ID NO: 263 (Cloning vector pPlatTET-gRNA2, Accession No. BAV54124).

(SEQ ID NO: 263
MDKKYSIGLAIGTNSVGWAVITDEYKVPSKKFKVLGNTDRHSIKK
NLIGALLFDSGETAEATRLKRTARRRYTRRKNRICYLQEIFSNEM
AKVDDSFFHRLEESFLVEEDKKHERHPIFGNIVDEVAYHEKYPTI
YHLRKKLVDSTDKADLRLIYLALAHMIKFRGHFLIEGDLNPDNSD
VDKLFIQLVQTYNQLFEENPINASGVDAKAILSARLSKSRRLENL
IAQLPGEKKNGLFGNLIALSLGLTPNFKSNFDLAEDAKLQLSKDT
YDDDLDNLLAQIGDQYADLFLAAKNLSDAILLSDILRVNTEITKA
PLSASMIKRYDEHHQDLTLLKALVRQQLPEKYKEIFFDQSKNGYA
GYIDGGASQEEFYKFIKPILEKMDGTEELLVKLNREDLLRKQRTF
DNGSIPHQIHLGELHAILRRQEDFYPFLKDNREKIEKILTFRIPY
YVGPLARGNSRFAWMTRKSEETITPWNFEEVVDKGASAQSFIERM
TNFDKNLPNEKVLPKHSLLYEYFTVYNELTKVKYVTEGMRKPAFL
SGEQKKAIVDLLFKTNRKVTVKQLKEDYFKKIECFDSVEISGVED
RFNASLGTYHDLLKIIKDKDFLDNEENEDILEDIVLTLTLFEDRE
MIEERLKTYAHLFDDKVMKQLKRRRYTGWGRLSRKLINGIRDKQS
GKTILDFLKSDGFANRNFMQLIHDDSLTFKEDIQKAQVSGQGDSL
HEHIANLAGSPAIKKGILQTVKVVDELVKVMGRHKPENIVIEMAR
ENQTTQKGQKNSRERMKRIEEGIKELGSQILKEHPVENTQLQNEK
LYLYYLQNGRDMYVDQELDINRLSDYDVDAIVPQSFLKDDSIDNK
VLTRSDKNRGKSDNVPSEEVVKKMKNYWRQLLNAKLITQRKFDNL
TKAERGGLSELDKAGFIKRQLVETRQITKHVAQILDSRMNTKYDE
NDKLIREVKVITLKSKLVSDFRKDFQFYKVREINNYHHAHDAYLN
AVVGTALIKKYPKLESEFVYGDYKVYDVRKMIAKSEQEIGKATAK
YFFYSNIMNFFKTEITLANGEIRKRPLIETNGETGEIVWDKGRDF
ATVRKVLSMPQVNIVKKTEVQTGGFSKESILPKRNSDKLIARKKD
WDPKKYGGFDSPTVAYSVLVVAKVEKGKSKKLKSVKELLGITIME
RSSFEKNPIDFLEAKGYKEVKKDLIIKLPKYSLFELENGRKRMLA
SAGELQKGNELALPSKYVNFLYLASHYEKLKGSPEDNEQKQLFVE
QHKHYLDEIIEQISEFSKRVILADANLDKVLSAYNKHRDKPIREQ
AENIIHLFTLTNLGAPAAFKYFDTTIDRKRYTSTKEVLDATLIHQ
SITGLYETRIDLSQLGGD;

see, e.g., Qi et al., Repurposing CRISPR as an RNA-guided platform for sequence-specific control of gene expression. Cell. 2013; 152(5):1173-83, the entire contents of which are incorporated herein by reference).

Additional suitable nuclease-inactive dCas9 domains will be apparent to those of skill in the art based on this disclosure and knowledge in the field, and are within the scope of this disclosure. Such additional exemplary suitable nuclease-inactive Cas9 domains include, but are not limited to, D10A/H840A, D10A/D839A/H840A, and D10A/D839A/H840A/N863A mutant domains (See, e.g., Prashant et al., CAS9 transcriptional activators for target specificity screening and paired nickases for cooperative genome engineering. Nature Biotechnology. 2013; 31(9): 833-838, the entire contents of which are incorporated herein by reference). In some embodiments the dCas9 domain comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the dCas9 domains provided herein. In some embodiments, the Cas9 domain comprises an amino acid sequences that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more or more mutations compared to any one of the amino acid sequences set forth in SEQ ID NOs: 10-263. In some embodiments, the Cas9 domain comprises an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences set forth in SEQ ID NOs: 10-263.

In some embodiments, the Cas9 domain is a Cas9 nickase. The Cas9 nickase may be a Cas9 protein that is capable of cleaving only one strand of a duplexed nucleic acid molecule (e.g., a duplexed DNA molecule). In some embodiments the Cas9 nickase cleaves the target strand of a duplexed nucleic acid molecule, meaning that the Cas9 nickase cleaves the strand that is base paired to (complementary to) a gRNA (e.g., an sgRNA) that is bound to the Cas9. In some embodiments, a Cas9 nickase comprises a D10A mutation and has a histidine at position 840 of SEQ ID NO: 10, or a mutation in any of SEQ ID NOs: 11-260. For example, a Cas9 nickase may comprise the amino acid sequence as set forth in SEQ ID NO: 674. In some embodiments the Cas9 nickase cleaves the non-target, non-base-edited strand of a duplexed nucleic acid molecule, meaning that the Cas9 nickase cleaves the strand that is not base paired to a gRNA (e.g., an sgRNA) that is bound to the Cas9. In some embodiments, a Cas9 nickase comprises an H840A mutation and has an aspartic acid residue at position 10 of SEQ ID NO: 10, or a corresponding mutation in any of SEQ ID NOs: 11-260. In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field, and are within the scope of this disclosure.

Cas9 Domains with Reduced PAM Exclusivity

Some aspects of the disclosure provide Cas9 domains that have different PAM specificities. Typically, Cas9 proteins, such as Cas9 from S. pyogenes (spCas9), require a canonical NGG PAM sequence to bind a particular nucleic acid region. This may limit the ability to edit desired bases within a genome. In some embodiments, the base editing fusion proteins provided herein may need to be placed at a precise location, for example where a target base is placed within a 4 base region (e.g., a “deamination window”), which is approximately 15 bases upstream of the PAM. See Komor, A. C., et al., “Programmable editing of a target base in genomic DNA without double-stranded DNA cleavage” Nature 533, 420-424 (2016), the entire contents of which are hereby incorporated by reference. Accordingly, in some embodiments, any of the fusion proteins provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the Cas9 domain is a Cas9 domain from Staphylococcus aureus (SaCas9). In some embodiments, the SaCas9 domain is a nuclease active SaCas9, a nuclease inactive SaCas9 (SaCas9d), or a SaCas9 nickase (SaCas9n). In some embodiments, the SaCas9 comprises the amino acid sequence SEQ ID NO: 4273. In some embodiments, the SaCas9 comprises a N579X mutation of SEQ ID NO: 4273, or a corresponding mutation in any of the amino acid sequences provided in SEQ ID NOs: 11-260, wherein X is any amino acid except for N. In some embodiments, the SaCas9 comprises a N579A mutation of SEQ ID NO: 4273, or a corresponding mutation in any of the amino acid sequences provided in SEQ ID NOs: 11-260. In some embodiments, the SaCas9 domain, the SaCas9d domain, or the SaCas9n domain can bind to a nucleic acid sequence having a non-canonical PAM. In some embodiments, the SaCas9 domain, the SaCas9d domain, or the SaCas9n domain can bind to a nucleic acid sequence having a NNGRRT PAM sequence. In some embodiments, the SaCas9 domain comprises one or more of a E781X, a N967X, and a R1014X mutation of SEQ ID NO: 4273, or a corresponding mutation in any of the amino acid sequences provided in SEQ ID NOs: 11-260, wherein X is any amino acid. In some embodiments, the SaCas9 domain comprises one or more of a E781K, a N967K, and a R1014H mutation of SEQ ID NO: 4273, or one or more corresponding mutation in any of the amino acid sequences provided in SEQ ID NOs: 11-260. In some embodiments, the SaCas9 domain comprises a E781K, a N967K, or a R1014H mutation of SEQ ID NO: 4273, or corresponding mutations in any of the amino acid sequences provided in SEQ ID NOs: 11-260.

In some embodiments, the Cas9 domain of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of SEQ ID NOs: 4273-4275. In some embodiments, the Cas9 domain of any of the fusion proteins provided herein comprises the amino acid sequence of any one of SEQ ID NOs: 4273-4275. In some embodiments, the Cas9 domain of any of the fusion proteins provided herein consists of the amino acid sequence of any one of SEQ ID NOs: 4273-4275.

Exemplary SaCas9 sequence
(SEQ ID NO: 4273)
KRNYILGLDIGITSVGYGIIDYETRDVIDAGVRLFKEANVENNEG
RRSKRGARRLKRRRRHRIQRVKKLLFDYNLLTDHSELSGINPYEA
RVKGLSQKLSEEEFSAALLHLAKRRGVHNVNEVEEDTGNELSTKE
QISRNSKALEEKYVAELQLERLKKDGEVRGSINRFKTSDYVKEAK
QLLKVQKAYHQLDQSFIDTYIDLLETRRTYYEGPGEGSPFGWKDI
KEWYEMLMGHCTYFPEELRSVKYAYNADLYNALNDLNNLVITRDE
NEKLEYYEKFQIIENVFKQKKKPTLKQIAKEILVNEEDIKGYRVT
STGKPEFTNLKVYHDIKDITARKEIIENAELLDQIAKILTIYQSS
EDIQEELTNLNSELTQEEIEQISNLKGYTGTHNLSLKAINLILDE
LWHTNDNQIAIFNRLKLVPKKVDLSQQKEIPTTLVDDFILSPVVK
RSFIQSIKVINAIIKKYGLPNDIIIELAREKNSKDAQKMINEMQK
RNRQTNERIEEIIRTTGKENAKYLIEKIKLHDMQEGKCLYSLEAI
PLEDLLNNPFNYEVDHIIPRSVSFDNSFNNKVLVKQEENSKKGNR
TPFQYLSSSDSKISYETFKKHILNLAKGKGRISKTKKEYLLEERD
INRFSVQKDFINRNLVDTRYATRGLMNLLRSYFRVNNLDVKVKSI
NGGFTSFLRRKWKFKKERNKGYKHHAEDALIIANADFIFKEWKKL
DKAKKVMENQMFEEKQAESMPEIETEQEYKEIFITPHQIKHIKDF
KDYKYSHRVDKKPNRELINDTLYSTRKDDKGNTLIVNNLNGLYDK
DNDKLKKLINKSPEKLLMYHHDPQTYQKLKLIMEQYGDEKNPLYK
YYEETGNYLTKYSKKDNGPVIKKIKYYGNKLNAHLDITDDYPNSR
NKVVKLSLKPYRFDVYLDNGVYKFVTVKNLDVIKKENYYEVNSKC
YEEAKKLKKISNQAEFIASFYNNDLIKINGELYRVIGVNNDLLNR
IEVNMIDITYREYLENMNDKRPPRIIKTIASKTQSIKKYSTDILG
NLYEVKSKKHPQIIKKG

Residue N579 of SEQ ID NO: 4273, which is underlined and in bold, may be mutated (e.g., to a A579) to yield a SaCas9 nickase.

Exemplary SaCas9n sequence
(SEQ ID NO: 4274)
KRNYILGLDIGITSVGYGIIDYETRDVIDAGVRLFKEANVENNEG
RRSKRGARRLKRRRRHRIQRVKKLLFDYNLLTDHSELSGINPYEA
RVKGLSQKLSEEEFSAALLHLAKRRGVHNVNEVEEDTGNELSTKE
QISRNSKALEEKYVAELQLERLKKDGEVRGSINRFKTSDYVKEAK
QLLKVQKAYHQLDQSFIDTYIDLLETRRTYYEGPGEGSPFGWKDI
KEWYEMLMGHCTYFPEELRSVKYAYNADLYNALNDLNNLVITRDE
NEKLEYYEKFQIIENVFKQKKKPTLKQIAKEILVNEEDIKGYRVT
STGKPEFTNLKVYHDIKDITARKEIIENAELLDQIAKILTIYQSS
EDIQEELTNLNSELTQEEIEQISNLKGYTGTHNLSLKAINLILDE
LWHTNDNQIAIFNRLKLVPKKVDLSQQKEIPTTLVDDFILSPVVK
RSFIQSIKVINAIIKKYGLPNDIIIELAREKNSKDAQKMINEMQK
RNRQTNERIEEIIRTTGKENAKYLIEKIKLHDMQEGKCLYSLEAI
PLEDLLNNPFNYEVDHIIPRSVSFDNSFNNKVLVKQEEASKKGNR
TPFQYLSSSDSKISYETFKKHILNLAKGKGRISKTKKEYLLEERD
INRFSVQKDFINRNLVDTRYATRGLMNLLRSYFRVNNLDVKVKSI
NGGFTSFLRRKWKFKKERNKGYKHHAEDALIIANADFIFKEWKKL
DKAKKVMENQMFEEKQAESMPEIETEQEYKEIFITPHQIKHIKDF
KDYKYSHRVDKKPNRELINDTLYSTRKDDKGNTLIVNNLNGLYDK
DNDKLKKLINKSPEKLLMYHHDPQTYQKLKLIMEQYGDEKNPLYK
YYEETGNYLTKYSKKDNGPVIKKIKYYGNKLNAHLDITDDYPNSR
NKVVKLSLKPYRFDVYLDNGVYKFVTVKNLDVIKKENYYEVNSKC
YEEAKKLKKISNQAEFIASFYNNDLIKINGELYRVIGVNNDLLNR
IEVNMIDITYREYLENMNDKRPPRIIKTIASKTQSIKKYSTDILG
NLYEVKSKKHPQIIKKG.

Residue A579 of SEQ ID NO: xx, which can be mutated from N579 of SEQ ID NO: 4274 to yield a SaCas9 nickase, is underlined and in bold.

Exemplary SaKKH Cas9
(SEQ ID NO: 4275)
KRNYILGLDIGITSVGYGIIDYETRDVIDAGVRLFKEANVENNEG
RRSKRGARRLKRRRRHRIQRVKKLLFDYNLLTDHSELSGINPYEA
RVKGLSQKLSEEEFSAALLHLAKRRGVHNVNEVEEDTGNELSTKE
QISRNSKALEEKYVAELQLERLKKDGEVRGSINRFKTSDYVKEAK
QLLKVQKAYHOLDQSFIDTYIDLLETRRTYYEGPGEGSPFGWKDI
KEWYEMLMGHCTYFPEELRSVKYAYNADLYNALNDLNNLVITRDE
NEKLEYYEKFQIIENVFKQKKKPTLKQIAKEILVNEEDIKGYRVT
STGKPEFTNLKVYHDIKDITARKEIIENAELLDQIAKILTIYQSS
EDIQEELTNLNSELTQEEIEQISNLKGYTGTHNLSLKAINLILDE
LWHTNDNQIAIFNRLKLVPKKVDLSQQKEIPTTLVDDFILSPVVK
RSFIQSIKVINAIIKKYGLPNDIIIELAREKNSKDAQKMINEMQK
RNRQTNERIEEIIRTTGKENAKYLIEKIKLHDMQEGKCLYSLEAI
PLEDLLNNPFNYEVDHIIPRSVSFDNSFNNKVLVKQEEASKKGNR
TPFQYLSSSDSKISYETFKKHILNLAKGKGRISKTKKEYLLEERD
INRFSVQKDFINRNLVDTRYATRGLMNLLRSYFRVNNLDVKVKSI
NGGFTSFLRRKWKFKKERNKGYKHHAEDALIIANADFIFKEWKKL
DKAKKVMENOMFEEKQAESMPEIETEQEYKEIFITPHQIKHIKDF
KDYKYSHRVDKKPNRKLINDTLYSTRKDDKGNTLIVNNLNGLYDK
DNDKLKKLINKSPEKLLMYHHDPQTYQKLKLIMEQYGDEKNPLYK
YYEETGNYLTKYSKKDNGPVIKKIKYYGNKLNAHLDITDDYPNSR
NKVVKLSLKPYRFDVYLDNGVYKFVTVKNLDVIKKENYYEVNSKC
YEEAKKLKKISNQAEFIASFYKNDLIKINGELYRVIGVNNDLLNR
IEVNMIDITYREYLENMNDKRPPHIIKTIASKTQSIKKYSTDILG
NLYEVKSKKHPQIIKKG.

Residue A579 of SEQ ID NO: 4275, which can be mutated from N579 of SEQ ID NO: 4275 to yield a SaCas9 nickase, is underlined and in bold. Residues K781, K967, and H1014 of SEQ ID NO: 4275, which can be mutated from E781, N967, and R1014 of SEQ ID NO: 4275 to yield a SaKKH Cas9 are underlined and in italics.

In some embodiments, the Cas9 domain is a Cas9 domain from Streptococcus pyogenes (SpCas9). In some embodiments, the SpCas9 domain is a nuclease active SpCas9, a nuclease inactive SpCas9 (SpCas9d), or a SpCas9 nickase (SpCas9n). In some embodiments, the SpCas9 comprises the amino acid sequence SEQ ID NO: 4276. In some embodiments, the SpCas9 comprises a D9X mutation of SEQ ID NO: 4276, or a corresponding mutation in any of the amino acid sequences provided in SEQ ID NOs: 11-260, wherein X is any amino acid except for D. In some embodiments, the SpCas9 comprises a D9A mutation of SEQ ID NO: 4276, or a corresponding mutation in any of the amino acid sequences provided in SEQ ID NOs: 11-260. In some embodiments, the SpCas9 domain, the SpCas9d domain, or the SpCas9n domain can bind to a nucleic acid sequence having a non-canonical PAM. In some embodiments, the SpCas9 domain, the SpCas9d domain, or the SpCas9n domain can bind to a nucleic acid sequence having a NGG, a NGA, or a NGCG PAM sequence. In some embodiments, the SpCas9 domain comprises one or more of a D1134X, a R1334X, and a T1336X mutation of SEQ ID NO: 4276, or a corresponding mutation in any of the amino acid sequences provided in SEQ ID NOs: 11-260, wherein X is any amino acid. In some embodiments, the SpCas9 domain comprises one or more of a D1134E, R1334Q, and T1336R mutation of SEQ ID NO: 4276, or a corresponding mutation in any of the amino acid sequences provided in SEQ ID NOs: 11-260. In some embodiments, the SpCas9 domain comprises a D1134E, a R1334Q, and a T1336R mutation of SEQ ID NO: 4276, or corresponding mutations in any of the amino acid sequences provided in SEQ ID NOs: 11-260. In some embodiments, the SpCas9 domain comprises one or more of a D1134X, a R1334X, and a T1336X mutation of SEQ ID NO: 4276, or a corresponding mutation in any of the amino acid sequences provided in SEQ ID NOs: 11-260, wherein X is any amino acid. In some embodiments, the SpCas9 domain comprises one or more of a D1134V, a R1334Q, and a T1336R mutation of SEQ ID NO: 4276, or a corresponding mutation in any of the amino acid sequences provided in SEQ ID NOs: 11-260. In some embodiments, the SpCas9 domain comprises a D1134V, a R1334Q, and a T1336R mutation of SEQ ID NO: 4276, or corresponding mutations in any of the amino acid sequences provided in SEQ ID NOs: 11-260. In some embodiments, the SpCas9 domain comprises one or more of a D1134X, a G1217X, a R1334X, and a T1336X mutation of SEQ ID NO: 4276, or a corresponding mutation in any of the amino acid sequences provided in SEQ ID NOs: 11-260, wherein X is any amino acid. In some embodiments, the SpCas9 domain comprises one or more of a D1134V, a G1217R, a R1334Q, and a T1336R mutation of SEQ ID NO: 4276, or a corresponding mutation in any of the amino acid sequences provided in SEQ ID NOs: 11-260. In some embodiments, the SpCas9 domain comprises a D1134V, a G1217R, a R1334Q, and a T1336R mutation of SEQ ID NO: 4276, or corresponding mutations in any of the amino acid sequences provided in SEQ ID NOs: 11-260.

In some embodiments, the Cas9 domain of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of SEQ ID NOs: 4276-4280. In some embodiments, the Cas9 domain of any of the fusion proteins provided herein comprises the amino acid sequence of any one of SEQ ID NOs: 4276-4280. In some embodiments, the Cas9 domain of any of the fusion proteins provided herein consists of the amino acid sequence of any one of SEQ ID NOs: 4276-4280.

Exemplary SpCas9
(SEQ ID NO: 4276)
DKKYSIGLDIGTNSVGWAVITDEYKVPSKKFKVLGNTDRHSIKKN
LIGALLFDSGETAEATRLKRTARRRYTRRKNRICYLQEIFSNEMA
KVDDSFFHRLEESFLVEEDKKHERHPIFGNIVDEVAYHEKYPTIY
HLRKKLVDSTDKADLRLIYLALAHMIKFRGHFLIEGDLNPDNSDV
DKLFIQLVQTYNQLFEENPINASGVDAKAILSARLSKSRRLENLI
AQLPGEKKNGLFGNLIALSLGLTPNFKSNFDLAEDAKLQLSKDTY
DDDLDNLLAQIGDQYADLFLAAKNLSDAILLSDILRVNTEITKAP
LSASMIKRYDEHHQDLTLLKALVRQQLPEKYKEIFFDQSKNGYAG
YIDGGASQEEFYKFIKPILEKMDGTEELLVKLNREDLLRKQRTFD
NGSIPHQIHLGELHAILRRQEDFYPFLKDNREKIEKILTFRIPYY
VGPLARGNSRFAWMTRKSEETITPWNFEEVVDKGASAQSFIERMT
NFDKNLPNEKVLPKHSLLYEYFTVYNELTKVKYVTEGMRKPAFLS
GEQKKAIVDLLFKTNRKVTVKQLKEDYFKKIECFDSVEISGVEDR
FNASLGTYHDLLKIIKDKDFLDNEENEDILEDIVLTLTLFEDREM
IEERLKTYAHLFDDKVMKQLKRRRYTGWGRLSRKLINGIRDKQSG
KTILDFLKSDGFANRNFMQLIHDDSLTFKEDIQKAQVSGQGDSLH
EHIANLAGSPAIKKGILQTVKVVDELVKVMGRHKPENIVIEMARE
NQTTQKGQKNSRERMKRIEEGIKELGSQILKEHPVENTQLQNEKL
YLYYLQNGRDMYVDQELDINRLSDYDVDHIVPQSFLKDDSIDNKV
LTRSDKNRGKSDNVPSEEVVKKMKNYWRQLLNAKLITQRKFDNLT
KAERGGLSELDKAGFIKRQLVETRQITKHVAQILDSRMNTKYDEN
DKLIREVKVITLKSKLVSDFRKDFQFYKVREINNYHHAHDAYLNA
VVGTALIKKYPKLESEFVYGDYKVYDVRKMIAKSEQEIGKATAKY
FFYSNIMNFFKTEITLANGEIRKRPLIETNGETGEIVWDKGRDFA
TVRKVLSMPQVNIVKKTEVQTGGFSKESILPKRNSDKLIARKKDW
DPKKYGGFDSPTVAYSVLVVAKVEKGKSKKLKSVKELLGITIMER
SSFEKNPIDFLEAKGYKEVKKDLIIKLPKYSLFELENGRKRMLAS
AGELQKGNELALPSKYVNFLYLASHYEKLKGSPEDNEQKQLFVEQ
HKHYLDEIIEQISEFSKRVILADANLDKVLSAYNKHRDKPIREQA
ENIIHLFTLTNLGAPAAFKYFDTTIDRKRYTSTKEVLDATLIHQS
ITGLYETRIDLSQLGGD
Exemplary SpCas9n
(SEQ ID NO: 4277)
DKKYSIGLAIGTNSVGWAVITDEYKVPSKKFKVLGNTDRHSIKKN
LIGALLFDSGETAEATRLKRTARRRYTRRKNRICYLQEIFSNEMA
KVDDSFFHRLEESFLVEEDKKHERHPIFGNIVDEVAYHEKYPTIY
HLRKKLVDSTDKADLRLIYLALAHMIKFRGHFLIEGDLNPDNSDV
DKLFIQLVQTYNQLFEENPINASGVDAKAILSARLSKSRRLENLI
AQLPGEKKNGLFGNLIALSLGLTPNFKSNFDLAEDAKLQLSKDTY
DDDLDNLLAQIGDQYADLFLAAKNLSDAILLSDILRVNTEITKAP
LSASMIKRYDEHHQDLTLLKALVRQQLPEKYKEIFFDQSKNGYAG
YIDGGASQEEFYKFIKPILEKMDGTEELLVKLNREDLLRKQRTFD
NGSIPHQIHLGELHAILRRQEDFYPFLKDNREKIEKILTFRIPYY
VGPLARGNSRFAWMTRKSEETITPWNFEEVVDKGASAQSFIERMT
NFDKNLPNEKVLPKHSLLYEYFTVYNELTKVKYVTEGMRKPAFLS
GEQKKAIVDLLFKTNRKVTVKQLKEDYFKKIECFDSVEISGVEDR
FNASLGTYHDLLKIIKDKDFLDNEENEDILEDIVLTLTLFEDREM
IEERLKTYAHLFDDKVMKQLKRRRYTGWGRLSRKLINGIRDKQSG
KTILDFLKSDGFANRNFMQLIHDDSLTFKEDIQKAQVSGQGDSLH
EHIANLAGSPAIKKGILQTVKVVDELVKVMGRHKPENIVIEMARE
NQTTQKGQKNSRERMKRIEEGIKELGSQILKEHPVENTQLQNEKL
YLYYLQNGRDMYVDQELDINRLSDYDVDHIVPQSFLKDDSIDNKV
LTRSDKNRGKSDNVPSEEVVKKMKNYWRQLLNAKLITQRKFDNLT
KAERGGLSELDKAGFIKRQLVETRQITKHVAQILDSRMNTKYDEN
DKLIREVKVITLKSKLVSDFRKDFQFYKVREINNYHHAHDAYLNA
VVGTALIKKYPKLESEFVYGDYKVYDVRKMIAKSEQEIGKATAKY
FFYSNIMNFFKTEITLANGEIRKRPLIETNGETGEIVWDKGRDFA
TVRKVLSMPQVNIVKKTEVQTGGFSKESILPKRNSDKLIARKKDW
DPKKYGGFDSPTVAYSVLVVAKVEKGKSKKLKSVKELLGITIMER
SSFEKNPIDFLEAKGYKEVKKDLIIKLPKYSLFELENGRKRMLAS
AGELQKGNELALPSKYVNFLYLASHYEKLKGSPEDNEQKQLFVEQ
HKHYLDEIIEQISEFSKRVILADANLDKVLSAYNKHRDKPIREQA
ENIIHLFTLTNLGAPAAFKYFDTTIDRKRYTSTKEVLDATLIHQS
ITGLYETRIDLSQLGGD
Exemplary SpEQR Cas9
(SEQ ID NO: 4278)
DKKYSIGLAIGTNSVGWAVITDEYKVPSKKFKVLGNTDRHSIKKN
LIGALLFDSGETAEATRLKRTARRRYTRRKNRICYLQEIFSNEMA
KVDDSFFHRLEESFLVEEDKKHERHPIFGNIVDEVAYHEKYPTI
YHLRKKLVDSTDKADLRLIYLALAHMIKFRGHFLIEGDLNPDNSD
VDKLFIQLVQTYNQLFEENPINASGVDAKAILSARLSKSRRLENL
IAQLPGEKKNGLFGNLIALSLGLTPNFKSNFDLAEDAKLQLSKDT
YDDDLDNLLAQIGDQYADLFLAAKNLSDAILLSDILRVNTEITKA
PLSASMIKRYDEHHQDLTLLKALVRQQLPEKYKEIFFDQSKNGYA
GYIDGGASQEEFYKFIKPILEKMDGTEELLVKLNREDLLRKQRTF
DNGSIPHQIHLGELHAILRRQEDFYPFLKDNREKIEKILTFRIPY
YVGPLARGNSRFAWMTRKSEETITPWNFEEVVDKGASAQSFIERM
TNFDKNLPNEKVLPKHSLLYEYFTVYNELTKVKYVTEGMRKPAFL
SGEQKKAIVDLLFKTNRKVTVKQLKEDYFKKIECFDSVEISGVED
RFNASLGTYHDLLKIIKDKDFLDNEENEDILEDIVLTLTLFEDRE
MIEERLKTYAHLFDDKVMKQLKRRRYTGWGRLSRKLINGIRDKQS
GKTILDFLKSDGFANRNFMQLIHDDSLTFKEDIQKAQVSGQGDSL
HEHIANLAGSPAIKKGILQTVKVVDELVKVMGRHKPENIVIEMAR
ENQTTQKGQKNSRERMKRIEEGIKELGSQILKEHPVENTQLQNEK
LYLYYLQNGRDMYVDQELDINRLSDYDVDHIVPQSFLKDDSIDNK
VLTRSDKNRGKSDNVPSEEVVKKMKNYWRQLLNAKLITQRKFDNL
TKAERGGLSELDKAGFIKRQLVETRQITKHVAQILDSRMNTKYDE
NDKLIREVKVITLKSKLVSDFRKDFQFYKVREINNYHHAHDAYLN
AVVGTALIKKYPKLESEFVYGDYKVYDVRKMIAKSEQEIGKATAK
YFFYSNIMNFFKTEITLANGEIRKRPLIETNGETGEIVWDKGRDF
ATVRKVLSMPQVNIVKKTEVQTGGFSKESILPKRNSDKLIARKKD
WDPKKYGGFESPTVAYSVLVVAKVEKGKSKKLKSVKELLGITIME
RSSFEKNPIDFLEAKGYKEVKKDLIIKLPKYSLFELENGRKRMLA
SAGELQKGNELALPSKYVNFLYLASHYEKLKGSPEDNEQKQLFVE
QHKHYLDEIIEQISEFSKRVILADANLDKVLSAYNKHRDKPIREQ
AENIIHLFTLTNLGAPAAFKYFDTTIDRKQYRSTKEVLDATLIHQ
SITGLYETRIDLSQLGGD

Residues E1134, Q1334, and R1336 of SEQ ID NO: 4278, which can be mutated from D1134, R1334, and T1336 of SEQ ID NO: 4278 to yield a SpEQR Cas9, are underlined and in bold.

Exemplary SpVQR Cas9
(SEQ ID NO: 4279)
DKKYSIGLAIGTNSVGWAVITDEYKVPSKKFKVLGNTDRHSIKKN
LIGALLFDSGETAEATRLKRTARRRYTRRKNRICYLQEIFSNEMA
KVDDSFFHRLEESFLVEEDKKHERHPIFGNIVDEVAYHEKYPTIY
HLRKKLVDSTDKADLRLIYLALAHMIKFRGHFLIEGDLNPDNSDV
DKLFIQLVQTYNQLFEENPINASGVDAKAILSARLSKSRRLENLI
AQLPGEKKNGLFGNLIALSLGLTPNFKSNFDLAEDAKLQLSKDTY
DDDLDNLLAQIGDQYADLFLAAKNLSDAILLSDILRVNTEITKAP
LSASMIKRYDEHHQDLTLLKALVRQQLPEKYKEIFFDQSKNGYAG
YIDGGASQEEFYKFIKPILEKMDGTEELLVKLNREDLLRKQRTFD
NGSIPHQIHLGELHAILRRQEDFYPFLKDNREKIEKILTFRIPYY
VGPLARGNSRFAWMTRKSEETITPWNFEEVVDKGASAQSFIERMT
NFDKNLPNEKVLPKHSLLYEYFTVYNELTKVKYVTEGMRKPAFLS
GEQKKAIVDLLFKTNRKVTVKQLKEDYFKKIECFDSVEISGVEDR
FNASLGTYHDLLKIIKDKDFLDNEENEDILEDIVLTLTLFEDREM
IEERLKTYAHLFDDKVMKQLKRRRYTGWGRLSRKLINGIRDKQSG
KTILDFLKSDGFANRNFMQLIHDDSLTFKEDIQKAQVSGQGDSLH
EHIANLAGSPAIKKGILQTVKVVDELVKVMGRHKPENIVIEMARE
NQTTQKGQKNSRERMKRIEEGIKELGSQILKEHPVENTQLQNEKL
YLYYLQNGRDMYVDQELDINRLSDYDVDHIVPQSFLKDDSIDNKV
LTRSDKNRGKSDNVPSEEVVKKMKNYWRQLLNAKLITQRKFDNLT
KAERGGLSELDKAGFIKRQLVETRQITKHVAQILDSRMNTKYDEN
DKLIREVKVITLKSKLVSDFRKDFQFYKVREINNYHHAHDAYLNA
VVGTALIKKYPKLESEFVYGDYKVYDVRKMIAKSEQEIGKATAKY
FFYSNIMNFFKTEITLANGEIRKRPLIETNGETGEIVWDKGRDFA
TVRKVLSMPQVNIVKKTEVQTGGFSKESILPKRNSDKLIARKKDW
DPKKYGGFVSPTVAYSVLVVAKVEKGKSKKLKSVKELLGITIMER
SSFEKNPIDFLEAKGYKEVKKDLIIKLPKYSLFELENGRKRMLAS
AGELQKGNELALPSKYVNFLYLASHYEKLKGSPEDNEQKQLFVEQ
HKHYLDEIIEQISEFSKRVILADANLDKVLSAYNKHRDKPIREQA
ENIIHLFTLTNLGAPAAFKYFDTTIDRKQYRSTKEVLDATLIHQS
ITGLYETRIDLSQLGGD

Residues V1134, Q1334, and R1336 of SEQ ID NO: 4279, which can be mutated from D1134, R1334, and T1336 of SEQ ID NO: 4279 to yield a SpVQR Cas9, are underlined and in bold.

Exemplary SpVRER Cas9
(SEQ ID NO: 4280)
DKKYSIGLAIGTNSVGWAVITDEYKVPSKKFKVLGNTDRHSIKKN
LIGALLFDSGETAEATRLKRTARRRYTRRKNRICYLQEIFSNEMA
KVDDSFFHRLEESFLVEEDKKHERHPIFGNIVDEVAYHEKYPTIY
HLRKKLVDSTDKADLRLIYLALAHMIKFRGHFLIEGDLNPDNSDV
DKLFIQLVQTYNQLFEENPINASGVDAKAILSARLSKSRRLENLI
AQLPGEKKNGLFGNLIALSLGLTPNFKSNFDLAEDAKLQLSKDTY
DDDLDNLLAQIGDQYADLFLAAKNLSDAILLSDILRVNTEITKAP
LSASMIKRYDEHHQDLTLLKALVRQQLPEKYKEIFFDQSKNGYAG
YIDGGASQEEFYKFIKPILEKMDGTEELLVKLNREDLLRKQRTFD
NGSIPHQIHLGELHAILRRQEDFYPFLKDNREKIEKILTFRIPYY
VGPLARGNSRFAWMTRKSEETITPWNFEEVVDKGASAQSFIERMT
NFDKNLPNEKVLPKHSLLYEYFTVYNELTKVKYVTEGMRKPAFLS
GEQKKAIVDLLFKTNRKVTVKQLKEDYFKKIECFDSVEISGVEDR
FNASLGTYHDLLKIIKDKDFLDNEENEDILEDIVLTLTLFEDREM
IEERLKTYAHLFDDKVMKQLKRRRYTGWGRLSRKLINGIRDKQSG
KTILDFLKSDGFANRNFMQLIHDDSLTFKEDIQKAQVSGQGDSLH
EHIANLAGSPAIKKGILQTVKVVDELVKVMGRHKPENIVIEMARE
NQTTQKGQKNSRERMKRIEEGIKELGSQILKEHPVENTQLQNEKL
YLYYLQNGRDMYVDQELDINRLSDYDVDHIVPQSFLKDDSIDNKV
LTRSDKNRGKSDNVPSEEVVKKMKNYWRQLLNAKLITQRKFDNLT
KAERGGLSELDKAGFIKRQLVETRQITKHVAQILDSRMNTKYDEN
DKLIREVKVITLKSKLVSDFRKDFQFYKVREINNYHHAHDAYLNA
VVGTALIKKYPKLESEFVYGDYKVYDVRKMIAKSEQEIGKATAKY
FFYSNIMNFFKTEITLANGEIRKRPLIETNGETGEIVWDKGRDFA
TVRKVLSMPQVNIVKKTEVQTGGFSKESILPKRNSDKLIARKKDW
DPKKYGGFVSPTVAYSVLVVAKVEKGKSKKLKSVKELLGITIMER
SSFEKNPIDFLEAKGYKEVKKDLIIKLPKYSLFELENGRKRMLAS
ARELQKGNELALPSKYVNFLYLASHYEKLKGSPEDNEQKQLFVEQ
HKHYLDEIIEQISEFSKRVILADANLDKVLSAYNKHRDKPIREQA
ENIIHLFTLTNLGAPAAFKYFDTTIDRKEYRSTKEVLDATLIHQS
ITGLYETRIDLSQLGGD

Residues V1134, R1217, Q1334, and R1336 of SEQ ID NO: 4280, which can be mutated from D1134, G1217, R1334, and T1336 of SEQ ID NO: 4280 to yield a SpVRER Cas9, are underlined and in bold.

The following are exemplary fusion proteins (e.g., base editing proteins) capable of binding to a nucleic acid sequence having a non-canonical (e.g., a non-NGG) PAM sequence:

Exemplary SaBE3 (rAPOBEC1-XTEN-SaCas9n-UGI-NLS)
(SEQ ID NO: 4281)
MSSETGPVAVDPTLRRRIEPHEFEVFFDPRELRKETCLLYEINWG
GRHSIWRHTSQNTNKHVEVNFIEKFTTERYFCPNTRCSITWFLSW
SPCGECSRAITEFLSRYPHVTLFIYIARLYHHADPRNRQGLRDLI
SSGVTIQIMTEQESGYCWRNFVNYSPSNEAHWPRYPHLWVRLYVL
ELYCIILGLPPCLNILRRKQPQLTFFTIALQSCHYQRLPPHILWA
TGLKSGSETPGTSESATPESKRNYILGLDIGITSVGYGIIDYETR
DVIDAGVRLFKEANVENNEGRRSKRGARRLKRRRRHRIQRVKKLL
FDYNLLTDHSELSGINPYEARVKGLSQKLSEEEFSAALLHLAKRR
GVHNVNEVEEDTGNELSTKEQISRNSKALEEKYVAELQLERLKKD
GEVRGSINRFKTSDYVKEAKQLLKVQKAYHQLDQSFIDTYIDLLE
TRRTYYEGPGEGSPFGWKDIKEWYEMLMGHCTYFPEELRSVKYAY
NADLYNALNDLNNLVITRDENEKLEYYEKFQIIENVFKQKKKPTL
KQIAKEILVNEEDIKGYRVTSTGKPEFTNLKVYHDIKDITARKEI
IENAELLDQIAKILTIYQSSEDIQEELTNLNSELTQEEIEQISNL
KGYTGTHNLSLKAINLILDELWHTNDNQIAIFNRLKLVPKKVDLS
QQKEIPTTLVDDFILSPVVKRSFIQSIKVINAIIKKYGLPNDIII
ELAREKNSKDAQKMINEMQKRNRQTNERIEEIIRTTGKENAKYLI
EKIKLHDMQEGKCLYSLEAIPLEDLLNNPFNYEVDHIIPRSVSFD
NSFNNKVLVKQEEASKKGNRTPFQYLSSSDSKISYETFKKHILNL
AKGKGRISKTKKEYLLEERDINRFSVQKDFINRNLVDTRYATRGL
MNLLRSYFRVNNLDVKVKSINGGFTSFLRRKWKFKKERNKGYKHH
AEDALIIANADFIFKEWKKLDKAKKVMENQMFEEKQAESMPEIET
EQEYKEIFITPHQIKHIKDFKDYKYSHRVDKKPNRELINDTLYST
RKDDKGNTLIVNNLNGLYDKDNDKLKKLINKSPEKLLMYHHDPQT
YQKLKLIMEQYGDEKNPLYKYYEETGNYLTKYSKKDNGPVIKKIK
YYGNKLNAHLDITDDYPNSRNKVVKLSLKPYRFDVYLDNGVYKFV
TVKNLDVIKKENYYEVNSKCYEEAKKLKKISNQAEFIASFYNNDL
IKINGELYRVIGVNNDLLNRIEVNMIDITYREYLENMNDKRPPRI
IKTIASKTQSIKKYSTDILGNLYEVKSKKHPQIIKKGSGGSTNLS
DIIEKETGKQLVIQESILMLPEEVEEVIGNKPESDILVHTAYDES
TDENVMLLTSDAPEYKPWALVIQDSNGENKIKMLSGGSPKKKRKV
Exemplary SaKKH-BE3
(rAPOBEC1-XTEN-SaCas9n-UGI-NLS)
(SEQ ID NO: 4282)
MSSETGPVAVDPTLRRRIEPHEFEVFFDPRELRKETCLLYEINWG
GRHSIWRHTSQNTNKHVEVNFIEKFTTERYFCPNTRCSITWFLSW
SPCGECSRAITEFLSRYPHVTLFIYIARLYHHADPRNRQGLRDLI
SSGVTIQIMTEQESGYCWRNFVNYSPSNEAHWPRYPHLWVRLYVL
ELYCIILGLPPCLNILRRKQPQLTFFTIALQSCHYQRLPPHILWA
TGLKSGSETPGTSESATPESKRNYILGLDIGITSVGYGIIDYETR
DVIDAGVRLFKEANVENNEGRRSKRGARRLKRRRRHRIQRVKKLL
FDYNLLTDHSELSGINPYEARVKGLSQKLSEEEFSAALLHLAKRR
GVHNVNEVEEDTGNELSTKEQISRNSKALEEKYVAELQLERLKKD
GEVRGSINRFKTSDYVKEAKQLLKVQKAYHQLDQSFIDTYIDLLE
TRRTYYEGPGEGSPFGWKDIKEWYEMLMGHCTYFPEELRSVKYAY
NADLYNALNDLNNLVITRDENEKLEYYEKFQIIENVFKQKKKPTL
KQIAKEILVNEEDIKGYRVTSTGKPEFTNLKVYHDIKDITARKEI
IENAELLDQIAKILTIYQSSEDIQEELTNLNSELTQEEIEQISNL
KGYTGTHNLSLKAINLILDELWHTNDNQIAIFNRLKLVPKKVDLS
QQKEIPTTLVDDFILSPVVKRSFIQSIKVINAIIKKYGLPNDIII
ELAREKNSKDAQKMINEMQKRNRQTNERIEEIIRTTGKENAKYLI
EKIKLHDMQEGKCLYSLEAIPLEDLLNNPFNYEVDHIIPRSVSFD
NSFNNKVLVKQEEASKKGNRTPFQYLSSSDSKISYETFKKHILNL
AKGKGRISKTKKEYLLEERDINRFSVQKDFINRNLVDTRYATRGL
MNLLRSYFRVNNLDVKVKSINGGFTSFLRRKWKFKKERNKGYKHH
AEDALIIANADFIFKEWKKLDKAKKVMENQMFEEKQAESMPEIET
EQEYKEIFITPHQIKHIKDFKDYKYSHRVDKKPNRKLINDTLYST
RKDDKGNTLIVNNLNGLYDKDNDKLKKLINKSPEKLLMYHHDPQT
YQKLKLIMEQYGDEKNPLYKYYEETGNYLTKYSKKDNGPVIKKIK
YYGNKLNAHLDITDDYPNSRNKVVKLSLKPYRFDVYLDNGVYKFV
TVKNLDVIKKENYYEVNSKCYEEAKKLKKISNQAEFIASFYKNDL
IKINGELYRVIGVNNDLLNRIEVNMIDITYREYLENMNDKRPPHI
IKTIASKTQSIKKYSTDILGNLYEVKSKKHPQIIKKGSGGSTNLS
DIIEKETGKQLVIQESILMLPEEVEEVIGNKPESDILVHTAYDES
TDENVMLLTSDAPEYKPWALVIQDSNGENKIKMLSGGSPKKKRKV
Exemplary EQR-BE3 (rAPOBEC1-XTEN-Cas9n-UGI-NLS)
(SEQ ID NO: 4283)
MSSETGPVAVDPTLRRRIEPHEFEVFFDPRELRKETCLLYEINWG
GRHSIWRHTSQNTNKHVEVNFIEKFTTERYFCPNTRCSITWFLSW
SPCGECSRAITEFLSRYPHVTLFIYIARLYHHADPRNRQGLRDLI
SSGVTIQIMTEQESGYCWRNFVNYSPSNEAHWPRYPHLWVRLYVL
ELYCIILGLPPCLNILRRKQPQLTFFTIALQSCHYQRLPPHILWA
TGLKSGSETPGTSESATPESDKKYSIGLAIGTNSVGWAVITDEYK
VPSKKFKVLGNTDRHSIKKNLIGALLFDSGETAEATRLKRTARRR
YTRRKNRICYLQEIFSNEMAKVDDSFFHRLEESFLVEEDKKHERH
PIFGNIVDEVAYHEKYPTIYHLRKKLVDSTDKADLRLIYLALAHM
IKFRGHFLIEGDLNPDNSDVDKLFIQLVQTYNQLFEENPINASGV
DAKAILSARLSKSRRLENLIAQLPGEKKNGLFGNLIALSLGLTPN
FKSNFDLAEDAKLQLSKDTYDDDLDNLLAQIGDQYADLFLAAKNL
SDAILLSDILRVNTEITKAPLSASMIKRYDEHHQDLTLLKALVRQ
QLPEKYKEIFFDQSKNGYAGYIDGGASQEEFYKFIKPILEKMDGT
EELLVKLNREDLLRKQRTFDNGSIPHQIHLGELHAILRRQEDFYP
FLKDNREKIEKILTFRIPYYVGPLARGNSRFAWMTRKSEETITPW
NFEEVVDKGASAQSFIERMTNFDKNLPNEKVLPKHSLLYEYFTVY
NELTKVKYVTEGMRKPAFLSGEQKKAIVDLLFKTNRKVTVKQLKE
DYFKKIECFDSVEISGVEDRFNASLGTYHDLLKIIKDKDFLDNEE
NEDILEDIVLTLTLFEDREMIEERLKTYAHLFDDKVMKQLKRRRY
TGWGRLSRKLINGIRDKQSGKTILDFLKSDGFANRNFMQLIHDDS
LTFKEDIQKAQVSGQGDSLHEHIANLAGSPAIKKGILQTVKVVDE
LVKVMGRHKPENIVIEMARENQTTQKGQKNSRERMKRIEEGIKEL
GSQILKEHPVENTQLQNEKLYLYYLQNGRDMYVDQELDINRLSDY
DVDHIVPQSFLKDDSIDNKVLTRSDKNRGKSDNVPSEEVVKKMKN
YWRQLLNAKLITQRKFDNLTKAERGGLSELDKAGFIKRQLVETRQ
ITKHVAQILDSRMNTKYDENDKLIREVKVITLKSKLVSDFRKDFQ
FYKVREINNYHHAHDAYLNAVVGTALIKKYPKLESEFVYGDYKVY
DVRKMIAKSEQEIGKATAKYFFYSNIMNFFKTEITLANGEIRKRP
LIETNGETGEIVWDKGRDFATVRKVLSMPQVNIVKKTEVQTGGFS
KESILPKRNSDKLIARKKDWDPKKYGGFESPTVAYSVLVVAKVEK
GKSKKLKSVKELLGITIMERSSFEKNPIDFLEAKGYKEVKKDLII
KLPKYSLFELENGRKRMLASAGELQKGNELALPSKYVNFLYLASH
YEKLKGSPEDNEQKQLFVEQHKHYLDEIIEQISEFSKRVILADAN
LDKVLSAYNKHRDKPIREQAENIIHLETLTNLGAPAAFKYFDTTI
DRKQYRSTKEVLDATLIHQSITGLYETRIDLSQLGGDSGGSTNLS
DIIEKETGKQLVIQESILMLPEEVEEVIGNKPESDILVHTAYDES
TDENVMLLTSDAPEYKPWALVIQDSNGENKIKMLSGGSPKKKRKV
VQR-BE3 (rAPOBEC1-XTEN-Cas9n-UGI-NLS)
(SEQ ID NO: 4284)
MSSETGPVAVDPTLRRRIEPHEFEVFFDPRELRKETCLLYEINWG
GRHSIWRHTSQNTNKHVEVNFIEKFTTERYFCPNTRCSITWFLSW
SPCGECSRAITEFLSRYPHVTLFIYIARLYHHADPRNRQGLRDLI
SSGVTIQIMTEQESGYCWRNFVNYSPSNEAHWPRYPHLWVRLYVL
ELYCIILGLPPCLNILRRKQPQLTFFTIALQSCHYQRLPPHILWA
TGLKSGSETPGTSESATPESDKKYSIGLAIGTNSVGWAVITDEYK
VPSKKFKVLGNTDRHSIKKNLIGALLFDSGETAEATRLKRTARRR
YTRRKNRICYLQEIFSNEMAKVDDSFFHRLEESFLVEEDKKHERH
PIFGNIVDEVAYHEKYPTIYHLRKKLVDSTDKADLRLIYLALAHM
IKFRGHFLIEGDLNPDNSDVDKLFIQLVQTYNQLFEENPINASGV
DAKAILSARLSKSRRLENLIAQLPGEKKNGLFGNLIALSLGLTPN
FKSNFDLAEDAKLQLSKDTYDDDLDNLLAQIGDQYADLFLAAKNL
SDAILLSDILRVNTEITKAPLSASMIKRYDEHHQDLTLLKALVRQ
QLPEKYKEIFFDQSKNGYAGYIDGGASQEEFYKFIKPILEKMDGT
EELLVKLNREDLLRKQRTFDNGSIPHQIHLGELHAILRRQEDFYP
FLKDNREKIEKILTFRIPYYVGPLARGNSRFAWMTRKSEETITPW
NFEEVVDKGASAQSFIERMTNFDKNLPNEKVLPKHSLLYEYFTVY
NELTKVKYVTEGMRKPAFLSGEQKKAIVDLLFKTNRKVTVKQLKE
DYFKKIECFDSVEISGVEDRFNASLGTYHDLLKIIKDKDFLDNEE
NEDILEDIVLTLTLFEDREMIEERLKTYAHLFDDKVMKQLKRRRY
TGWGRLSRKLINGIRDKQSGKTILDFLKSDGFANRNFMQLIHDDS
LTFKEDIQKAQVSGQGDSLHEHIANLAGSPAIKKGILQTVKVVDE
LVKVMGRHKPENIVIEMARENQTTQKGQKNSRERMKRIEEGIKEL
GSQILKEHPVENTQLQNEKLYLYYLQNGRDMYVDQELDINRLSDY
DVDHIVPQSFLKDDSIDNKVLTRSDKNRGKSDNVPSEEVVKKMKN
YWRQLLNAKLITQRKFDNLTKAERGGLSELDKAGFIKRQLVETRQ
ITKHVAQILDSRMNTKYDENDKLIREVKVITLKSKLVSDFRKDFQ
FYKVREINNYHHAHDAYLNAVVGTALIKKYPKLESEFVYGDYKVY
DVRKMIAKSEQEIGKATAKYFFYSNIMNFFKTEITLANGEIRKRP
LIETNGETGEIVWDKGRDFATVRKVLSMPQVNIVKKTEVQTGGFS
KESILPKRNSDKLIARKKDWDPKKYGGFVSPTVAYSVLVVAKVEK
GKSKKLKSVKELLGITIMERSSFEKNPIDFLEAKGYKEVKKDLII
KLPKYSLFELENGRKRMLASAGELQKGNELALPSKYVNFLYLASH
YEKLKGSPEDNEQKQLFVEQHKHYLDEIIEQISEFSKRVILADAN
LDKVLSAYNKHRDKPIREQAENIIHLFTLTNLGAPAAFKYFDTTI
DRKQYRSTKEVLDATLIHQSITGLYETRIDLSQLGGDSGGSTNLS
DIIEKETGKQLVIQESILMLPEEVEEVIGNKPESDILVHTAYDES
TDENVMLLTSDAPEYKPWALVIQDSNGENKIKMLSGGSPKKKRKV
VRER-BE3 (rAPOBEC1-XTEN-Cas9n-UGI-NLS)
(SEQ ID NO: 4285)
MSSETGPVAVDPTLRRRIEPHEFEVFFDPRELRKETCLLYEINWG
GRHSIWRHTSQNTNKHVEVNFIEKFTTERYFCPNTRCSITWFLSW
SPCGECSRAITEFLSRYPHVTLFIYIARLYHHADPRNRQGLRDLI
SSGVTIQIMTEQESGYCWRNFVNYSPSNEAHWPRYPHLWVRLYVL
ELYCIILGLPPCLNILRRKQPQLTFFTIALQSCHYQRLPPHILWA
TGLKSGSETPGTSESATPESDKKYSIGLAIGTNSVGWAVITDEYK
VPSKKFKVLGNTDRHSIKKNLIGALLFDSGETAEATRLKRTARRR
YTRRKNRICYLQEIFSNEMAKVDDSFFHRLEESFLVEEDKKHERH
PIFGNIVDEVAYHEKYPTIYHLRKKLVDSTDKADLRLIYLALAHM
IKFRGHFLIEGDLNPDNSDVDKLFIQLVQTYNQLFEENPINASGV
DAKAILSARLSKSRRLENLIAQLPGEKKNGLFGNLIALSLGLTPN
FKSNFDLAEDAKLQLSKDTYDDDLDNLLAQIGDQYADLFLAAKNL
SDAILLSDILRVNTEITKAPLSASMIKRYDEHHQDLTLLKALVRQ
QLPEKYKEIFFDQSKNGYAGYIDGGASQEEFYKFIKPILEKMDGT
EELLVKLNREDLLRKQRTFDNGSIPHQIHLGELHAILRRQEDFYP
FLKDNREKIEKILTFRIPYYVGPLARGNSRFAWMTRKSEETITPW
NFEEVVDKGASAQSFIERMTNFDKNLPNEKVLPKHSLLYEYFTVY
NELTKVKYVTEGMRKPAFLSGEQKKAIVDLLFKTNRKVTVKQLKE
DYFKKIECFDSVEISGVEDRFNASLGTYHDLLKIIKDKDFLDNEE
NEDILEDIVLTLTLFEDREMIEERLKTYAHLFDDKVMKQLKRRRY
TGWGRLSRKLINGIRDKQSGKTILDFLKSDGFANRNFMQLIHDDS
LTFKEDIQKAQVSGQGDSLHEHIANLAGSPAIKKGILQTVKVVDE
LVKVMGRHKPENIVIEMARENQTTQKGQKNSRERMKRIEEGIKEL
GSQILKEHPVENTQLQNEKLYLYYLQNGRDMYVDQELDINRLSDY
DVDHIVPQSFLKDDSIDNKVLTRSDKNRGKSDNVPSEEVVKKMKN
YWRQLLNAKLITQRKFDNLTKAERGGLSELDKAGFIKRQLVETRQ
ITKHVAQILDSRMNTKYDENDKLIREVKVITLKSKLVSDFRKDFQ
FYKVREINNYHHAHDAYLNAVVGTALIKKYPKLESEFVYGDYKVY
DVRKMIAKSEQEIGKATAKYFFYSNIMNFFKTEITLANGEIRKRP
LIETNGETGEIVWDKGRDFATVRKVLSMPQVNIVKKTEVQTGGFS
KESILPKRNSDKLIARKKDWDPKKYGGFVSPTVAYSVLVVAKVEK
GKSKKLKSVKELLGITIMERSSFEKNPIDFLEAKGYKEVKKDLII
KLPKYSLFELENGRKRMLASARELQKGNELALPSKYVNFLYLASH
YEKLKGSPEDNEQKQLFVEQHKHYLDEIIEQISEFSKRVILADAN
LDKVLSAYNKHRDKPIREQAENIIHLFTLTNLGAPAAFKYFDTTI
DRKEYRSTKEVLDATLIHQSITGLYETRIDLSQLGGDSGGSTNLS
DIIEKETGKQLVIQESILMLPEEVEEVIGNKPESDILVHTAYDES
TDENVMLLTSDAPEYKPWALVIQDSNGENKIKMLSGGSPKKKRKV

### High Fidelity Base Editors

Some aspects of the disclosure provide Cas9 fusion proteins (e.g., any of the fusion proteins provided herein) comprising a Cas9 domain that has high fidelity. Additional aspects of the disclosure provide Cas9 fusion proteins (e.g., any of the fusion proteins provided herein) comprising a Cas9 domain with decreased electrostatic interactions between the Cas9 domain and a sugar-phosphate backbone of a DNA, as compared to a wild-type Cas9 domain. In some embodiments, a Cas9 domain (e.g., a wild type Cas9 domain) comprises one or more mutations that decreases the association between the Cas9 domain and a sugar-phosphate backbone of a DNA. In some embodiments, any of the Cas9 fusion proteins provided herein comprise one or more of a N497X, a R661X, a Q695X, and/or a Q926X mutation of the amino acid sequence provided in SEQ ID NO: 10, or a corresponding mutation in any of the amino acid sequences provided in SEQ ID NOs: 11-260, wherein X is any amino acid. In some embodiments, any of the Cas9 fusion proteins provided herein comprise one or more of a N497A, a R661A, a Q695A, and/or a Q926A mutation of the amino acid sequence provided in SEQ ID NO: 10, or a corresponding mutation in any of the amino acid sequences provided in SEQ ID NOs: 11-260. In some embodiments, the Cas9 domain comprises a D10A mutation of the amino acid sequence provided in SEQ ID NO: 10, or a corresponding mutation in any of the amino acid sequences provided in SEQ ID NOs: 11-260. In some embodiments, the Cas9 domain (e.g., of any of the fusion proteins provided herein) comprises the amino acid sequence as set forth in SEQ ID NO: 325. In some embodiments, the fusion protein comprises the amino acid sequence as set forth in SEQ ID NO: 285. Cas9 domains with high fidelity are known in the art and would be apparent to the skilled artisan. For example, Cas9 domains with high fidelity have been described in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each are incorporated herein by reference.

It should be appreciated that the base editors provided herein, for example base editor 2 (BE2) or base editor 3 (BE3), may be converted into high fidelity base editors by modifying the Cas9 domain as described herein to generate high fidelity base editors, for example high fidelity base editor 2 (HF-BE2) or high fidelity base editor 3 (HF-BE3). In some embodiments, base editor 2 (BE2) comprises a deaminase domain, a dCas9, and a UGI domain. In some embodiments, base editor 3 (BE3) comprises a deaminase domain an nCas9 domain and a UGI domain.

Cas9 domain where mutations relative to Cas9
of SEQ ID NO: 10 are shown in bold and
underlines
(SEQ ID NO: 325)
DKKYSIGLAIGTNSVGWAVITDEYKVPSKKFKVLGNTDRHSIKKN
LIGALLFDSGETAEATRLKRTARRRYTRRKNRICYLQEIFSNEMA
KVDDSFFHRLEESFLVEEDKKHERHPIFGNIVDEVAYHEKYPTIY
HLRKKLVDSTDKADLRLIYLALAHMIKFRGHFLIEGDLNPDNSDV
DKLFIQLVQTYNQLFEENPINASGVDAKAILSARLSKSRRLENLI
AQLPGEKKNGLFGNLIALSLGLTPNFKSNFDLAEDAKLQLSKDTY
DDDLDNLLAQIGDQYADLFLAAKNLSDAILLSDILRVNTEITKAP
LSASMIKRYDEHHQDLTLLKALVRQQLPEKYKEIFFDQSKNGYAG
YIDGGASQEEFYKFIKPILEKMDGTEELLVKLNREDLLRKQRTFD
NGSIPHQIHLGELHAILRRQEDFYPFLKDNREKIEKILTFRIPYY
VGPLARGNSRFAWMTRKSEETITPWNFEEVVDKGASAQSFIERMT
AFDKNLPNEKVLPKHSLLYEYFTVYNELTKVKYVTEGMRKPAFLS
GEQKKAIVDLLFKTNRKVTVKQLKEDYFKKIECFDSVEISGVEDR
FNASLGTYHDLLKIIKDKDFLDNEENEDILEDIVLTLTLFEDREM
IEERLKTYAHLFDDKVMKQLKRRRYTGWGALSRKLINGIRDKQSG
KTILDFLKSDGFANRNFMALIHDDSLTFKEDIQKAQVSGQGDSLH
EHIANLAGSPAIKKGILQTVKVVDELVKVMGRHKPENIVIEMARE
NQTTQKGQKNSRERMKRIEEGIKELGSQILKEHPVENTQLQNEKL
YLYYLQNGRDMYVDQELDINRLSDYDVDHIVPQSFLKDDSIDNKV
LTRSDKNRGKSDNVPSEEVVKKMKNYWRQLLNAKLITQRKFDNLT
KAERGGLSELDKAGFIKRQLVETRAITKHVAQILDSRMNTKYDEN
DKLIREVKVITLKSKLVSDFRKDFQFYKVREINNYHHAHDAYLNA
VVGTALIKKYPKLESEFVYGDYKVYDVRKMIAKSEQEIGKATAKY
FFYSNIMNFFKTEITLANGEIRKRPLIETNGETGEIVWDKGRDFA
TVRKVLSMPQVNIVKKTEVQTGGFSKESILPKRNSDKLIARKKDW
DPKKYGGFDSPTVAYSVLVVAKVEKGKSKKLKSVKELLGITIMER
SSFEKNPIDFLEAKGYKEVKKDLIIKLPKYSLFELENGRKRMLAS
AGELQKGNELALPSKYVNFLYLASHYEKLKGSPEDNEQKQLFVEQ
HKHYLDEIIEQISEFSKRVILADANLDKVLSAYNKHRDKPIREQA
ENIIHLFTLTNLGAPAAFKYFDTTIDRKRYTSTKEVLDATLIHQS
ITGLYETRIDLSQLGGD
HF-BE3
(SEQ ID NO: 285)
MSSETGPVAVDPTLRRRIEPHEFEVFFDPRELRKETCLLYEINWG
GRHSIWRHTSQNTNKHVEVNFIEKFTTERYFCPNTRCSITWFLSW
SPCGECSRAITEFLSRYPHVTLFIYIARLYHHADPRNRQGLRDLI
SSGVTIQIMTEQESGYCWRNFVNYSPSNEAHWPRYPHLWVRLYVL
ELYCIILGLPPCLNILRRKQPQLTFFTIALQSCHYQRLPPHILWA
TGLKSGSETPGTSESATPESDKKYSIGLAIGTNSVGWAVITDEYK
VPSKKFKVLGNTDRHSIKKNLIGALLFDSGETAEATRLKRTARRR
YTRRKNRICYLQEIFSNEMAKVDDSFFHRLEESFLVEEDKKHERH
PIFGNIVDEVAYHEKYPTIYHLRKKLVDSTDKADLRLIYLALAHM
IKFRGHFLIEGDLNPDNSDVDKLFIQLVQTYNQLFEENPINASGV
DAKAILSARLSKSRRLENLIAQLPGEKKNGLFGNLIALSLGLTPN
FKSNFDLAEDAKLQLSKDTYDDDLDNLLAQIGDQYADLFLAAKNL
SDAILLSDILRVNTEITKAPLSASMIKRYDEHHQDLTLLKALVRQ
QLPEKYKEIFFDQSKNGYAGYIDGGASQEEFYKFIKPILEKMDGT
EELLVKLNREDLLRKQRTFDNGSIPHQIHLGELHAILRRQEDFYP
FLKDNREKIEKILTFRIPYYVGPLARGNSRFAWMTRKSEETITPW
NFEEVVDKGASAQSFIERMTAFDKNLPNEKVLPKHSLLYEYFTVY
NELTKVKYVTEGMRKPAFLSGEQKKAIVDLLFKTNRKVTVKQLKE
DYFKKIECFDSVEISGVEDRFNASLGTYHDLLKIIKDKDFLDNEE
NEDILEDIVLTLTLFEDREMIEERLKTYAHLFDDKVMKQLKRRRY
TGWGALSRKLINGIRDKQSGKTILDFLKSDGFANRNFMALIHDDS
LTFKEDIQKAQVSGQGDSLHEHIANLAGSPAIKKGILQTVKVVDE
LVKVMGRHKPENIVIEMARENQTTQKGQKNSRERMKRIEEGIKEL
GSQILKEHPVENTQLQNEKLYLYYLQNGRDMYVDQELDINRLSDY
DVDHIVPQSFLKDDSIDNKVLTRSDKNRGKSDNVPSEEVVKKMKN
YWRQLLNAKLITQRKFDNLTKAERGGLSELDKAGFIKRQLVETRA
ITKHVAQILDSRMNTKYDENDKLIREVKVITLKSKLVSDFRKDFQ
FYKVREINNYHHAHDAYLNAVVGTALIKKYPKLESEFVYGDYKVY
DVRKMIAKSEQEIGKATAKYFFYSNIMNFFKTEITLANGEIRKRP
LIETNGETGEIVWDKGRDFATVRKVLSMPQVNIVKKTEVQTGGFS
KESILPKRNSDKLIARKKDWDPKKYGGFDSPTVAYSVLVVAKVEK
GKSKKLKSVKELLGITIMERSSFEKNPIDFLEAKGYKEVKKDLII
KLPKYSLFELENGRKRMLASAGELQKGNELALPSKYVNFLYLASH
YEKLKGSPEDNEQKQLFVEQHKHYLDEIIEQISEFSKRVILADAN
LDKVLSAYNKHRDKPIREQAENIIHLFTLTNLGAPAAFKYFDTTI
DRKRYTSTKEVLDATLIHQSITGLYETRIDLSQLGGD

### Cas9 Fusion Proteins

Any of the Cas9 domains (e.g., a nuclease active Cas9 protein, a nuclease-inactive dCas9 protein, or a Cas9 nickase protein) disclosed herein may be fused to a second protein, thus fusion proteins provided herein comprise a Cas9 domain as provided herein and a second protein, or a “fusion partner”. In some embodiments, the second protein is fused to the N-terminus of the Cas9 domain. However, in other embodiments, the second protein is fused to the C-terminus of the Cas9 domain. In some embodiments, the second protein that is fused to the Cas9 domain is a nucleic acid editing domain. In some embodiments, the Cas9 domain and the nucleic acid editing domain are fused via a linker, while in other embodiments the Cas9 domain and the nucleic acid editing domain are fused directly to one another. In some embodiments, the linker comprises (GGGS)ₙ (SEQ ID NO: 265), (GGGGS)ₙ (SEQ ID NO: 5), (G)ₙ, (EAAAK)ₙ (SEQ ID NO: 6), (GGS)ₙ, (SGGS)ₙ (SEQ ID NO: 4288), SGSETPGTSESATPES (SEQ ID NO: 7), or (XP)ₙ motif, or a combination of any of these, wherein n is independently an integer between 1 and 30, and wherein X is any amino acid. In some embodiments, the linker comprises a (GGS)ₙ motif, wherein n is 1, 3, or 7. In some embodiments, the linker comprises a (GGS)ₙ motif, wherein n is 1, 2, 3, 4, 5, 6, 7, 8, 9, 10, 11, 12, 13, 14, or 15. In some embodiments, the linker comprises an amino acid sequence of SGSETPGTSESATPES (SEQ ID NO: 7),also referred to as the XTEN linker in the Examples). The length of the linker can influence the base to be edited, as illustrated in the Examples. For example, a linker of 3-amino-acid long (e.g., (GGS)₁) may give a 2-5, 2-4, 2-3, 3-4 base editing window relative to the PAM sequence, while a 9-amino-acid linker (e.g., (GGS)₃ (SEQ ID NO: 596)) may give a 2-6, 2-5, 2-4, 2-3, 3-6, 3-5, 3-4, 4-6, 4-5, 5-6 base editing window relative to the PAM sequence. A 16-amino-acid linker (e.g., the XTEN linker) may give a 2-7, 2-6, 2-5, 2-4, 2-3, 3-7, 3-6, 3-5, 3-4, 4-7, 4-6, 4-5, 5-7, 5-6, 6-7 base window relative to the PAM sequence with exceptionally strong activity, and a 21-amino-acid linker (e.g., (GGS)₇ (SEQ ID NO: 597)) may give a 3-8, 3-7, 3-6, 3-5, 3-4, 4-8, 4-7, 4-6, 4-5, 5-8, 5-7, 5-6, 6-8, 6-7, 7-8 base editing window relative to the PAM sequence. The novel finding that varying linker length may allow the dCas9 fusion proteins of the disclosure to edit nucleobases different distances from the PAM sequence affords significant clinical importance, since a PAM sequence may be of varying distance to the disease-causing mutation to be corrected in a gene. It is to be understood that the linker lengths described as examples here are not meant to be limiting.

In some embodiments, the second protein comprises an enzymatic domain. In some embodiments, the enzymatic domain is a nucleic acid editing domain. Such a nucleic acid editing domain may be, without limitation, a nuclease, a nickase, a recombinase, a deaminase, a methyltransferase, a methylase, an acetylase, or an acetyltransferase. Non-limiting exemplary binding domains that may be used in accordance with this disclosure include transcriptional activator domains and transcriptional repressor domains.

### Deaminase Domains

In some embodiments, second protein comprises a nucleic acid editing domain. In some embodiments, the nucleic acid editing domain can catalyze a C to U base change. In some embodiments, the nucleic acid editing domain is a deaminase domain. In some embodiments, the deaminase is a cytidine deaminase or a cytidine deaminase. In some embodiments, the deaminase is an apolipoprotein B mRNA-editing complex (APOBEC) family deaminase. In some embodiments, the deaminase is an APOBEC1 deaminase. In some embodiments, the deaminase is an APOBEC2 deaminase. In some embodiments, the deaminase is an APOBEC3 deaminase. In some embodiments, the deaminase is an APOBEC3A deaminase. In some embodiments, the deaminase is an APOBEC3B deaminase. In some embodiments, the deaminase is an APOBEC3C deaminase. In some embodiments, the deaminase is an APOBEC3D deaminase. In some embodiments, the deaminase is an APOBEC3E deaminase. In some embodiments, the deaminase is an APOBEC3F deaminase. In some embodiments, the deaminase is an APOBEC3G deaminase. In some embodiments, the deaminase is an APOBEC3H deaminase. In some embodiments, the deaminase is an APOBEC4 deaminase. In some embodiments, the deaminase is an activation-induced deaminase (AID). In some embodiments, the deaminase is a vertebrate deaminase. In some embodiments, the deaminase is an invertebrate deaminase. In some embodiments, the deaminase is a human, chimpanzee, gorilla, monkey, cow, dog, rat, or mouse deaminase. In some embodiments, the deaminase is a human deaminase. In some embodiments, the deaminase is a rat deaminase, e.g., rAPOBEC1. In some embodiments, the deaminase is a Petromyzon marinus cytidine deaminase 1 (pmCDA1). In some embodiments, the deminase is a human APOBEC3G (SEQ ID NO: 275). In some embodiments, the deaminase is a fragment of the human APOBEC3G (SEQ ID NO: 5740). In some embodiments, the deaminase is a human APOBEC3G variant comprising a D316R_D317R mutation (SEQ ID NO: 5739). In some embodiments, the deaminase is a frantment of the human APOBEC3G and comprising mutations corresponding to the D316R_D317R mutations in SEQ ID NO: 275 (SEQ ID NO: 5741).

In some embodiments, the nucleic acid editing domain is at least 80%, at least 85%, at least 90%, at least 92%, at least 95%, at least 96%, at least 97%, at least 98%, at least 99%, or at least 99.5% identical to the deaminase domain of any one of SEQ ID NOs: 266-284, 607-610, 5724-5736, or 5738-5741. In some embodiments, the nucleic acid editing domain comprises the amino acid sequence of any one of SEQ ID NOs: 266-284, 607-610, 5724-5736, or 5738-5741.

Deaminase Domains that Modulate the Editing Window of Base Editors

Some aspects of the disclosure are based on the recognition that modulating the deaminase domain catalytic activity of any of the fusion proteins provided herein, for example by making point mutations in the deaminase domain, affect the processivity of the fusion proteins (e.g., base editors). For example, mutations that reduce, but do not eliminate, the catalytic activity of a deaminase domain within a base editing fusion protein can make it less likely that the deaminase domain will catalyze the deamination of a residue adjacent to a target residue, thereby narrowing the deamination window. The ability to narrow the deamination window may prevent unwanted deamination of residues adjacent of specific target residues, which may decrease or prevent off-target effects.

In some embodiments, any of the fusion proteins provided herein comprise a deaminase domain (e.g., a cytidine deaminase domain) that has reduced catalytic deaminase activity. In some embodiments, any of the fusion proteins provided herein comprise a deaminase domain (e.g., a cytidine deaminase domain) that has a reduced catalytic deaminase activity as compared to an appropriate control. For example, the appropriate control may be the deaminase activity of the deaminase prior to introducing one or more mutations into the deaminase. In other embodiments, the appropriate control may be a wild-type deaminase. In some embodiments, the appropriate control is a wild-type apolipoprotein B mRNA-editing complex (APOBEC) family deaminase. In some embodiments, the appropriate control is an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, or an APOBEC3H deaminase. In some embodiments, the appropriate control is an activation induced deaminase (AID). In some embodiments, the appropriate control is a cytidine deaminase 1 from Petromyzon marinus (pmCDA1). In some embodiments, the deaminse domain may be a deaminase domain that has at least 1%, at least 5%, at least 15%, at least 20%, at least 25%, at least 30%, at least 40%, at least 50%, at least 60%, at lest 70%, at least 80%, at least 90%, or at least 95% less catalytic deaminase activity as compared to an appropriate control.

In some embodiments, any of the fusion proteins provided herein comprise an APOBEC deaminase comprising one or more mutations selected from the group consisting of H121X, H122X, R126X, R126X, R118X, W90X, W90X, and R132X of rAPOBEC1 (SEQ ID NO: 284), or one or more corresponding mutations in another APOBEC deaminase, wherein X is any amino acid. In some embodiments, any of the fusion proteins provided herein comprise an APOBEC deaminase comprising one or more mutations selected from the group consisting of H121R, H122R, R126A, R126E, R118A, W90A, W90Y, and R132E of rAPOBEC1 (SEQ ID NO: 284), or one or more corresponding mutations in another APOBEC deaminase.

In some embodiments, any of the fusion proteins provided herein comprise an APOBEC deaminase comprising one or more mutations selected from the group consisting of D316X, D317X, R320X, R320X, R313X, W285X, W285X, R326X of hAPOBEC3G (SEQ ID NO: 275), or one or more corresponding mutations in another APOBEC deaminase, wherein X is any amino acid. In some embodiments, any of the fusion proteins provided herein comprise an APOBEC deaminase comprising one or more mutations selected from the group consisting of D316R, D317R, R320A, R320E, R313A, W285A, W285Y, R326E of hAPOBEC3G (SEQ ID NO: 275), or one or more corresponding mutations in another APOBEC deaminase.

In some embodiments, any of the fusion proteins provided herein comprise an APOBEC deaminase comprising a H121R and a H122R mutation of rAPOBEC1 (SEQ ID NO: 284), or one or more corresponding mutations in another APOBEC deaminase. In some embodiments, any of the fusion proteins provided herein comprise an APOBEC deaminase comprising a R126A mutation of rAPOBEC1 (SEQ ID NO: 284), or one or more corresponding mutations in another APOBEC deaminase. In some embodiments, any of the fusion proteins provided herein comprise an APOBEC deaminase comprising a R126E mutation of rAPOBEC1 (SEQ ID NO: 284), or one or more corresponding mutations in another APOBEC deaminase. In some embodiments, any of the fusion proteins provided herein comprise an APOBEC deaminase comprising a R118A mutation of rAPOBEC1 (SEQ ID NO: 284), or one or more corresponding mutations in another APOBEC deaminase. In some embodiments, any of the fusion proteins provided herein comprise an APOBEC deaminase comprising a W90A mutation of rAPOBEC1 (SEQ ID NO: 284), or one or more corresponding mutations in another APOBEC deaminase. In some embodiments, any of the fusion proteins provided herein comprise an APOBEC deaminase comprising a W90Y mutation of rAPOBEC1 (SEQ ID NO: 284), or one or more corresponding mutations in another APOBEC deaminase. In some embodiments, any of the fusion proteins provided herein comprise an APOBEC deaminase comprising a R132E mutation of rAPOBEC1 (SEQ ID NO: 284), or one or more corresponding mutations in another APOBEC deaminase. In some embodiments, any of the fusion proteins provided herein comprise an APOBEC deaminase comprising a W90Y and a R126E mutation of rAPOBEC1 (SEQ ID NO: 284), or one or more corresponding mutations in another APOBEC deaminase. In some embodiments, any of the fusion proteins provided herein comprise an APOBEC deaminase comprising a R126E and a R132E mutation of rAPOBEC1 (SEQ ID NO: 284), or one or more corresponding mutations in another APOBEC deaminase. In some embodiments, any of the fusion proteins provided herein comprise an APOBEC deaminase comprising a W90Y and a R132E mutation of rAPOBEC1 (SEQ ID NO: 284), or one or more corresponding mutations in another APOBEC deaminase. In some embodiments, any of the fusion proteins provided herein comprise an APOBEC deaminase comprising a W90Y, R126E, and R132E mutation of rAPOBEC1 (SEQ ID NO: 284), or one or more corresponding mutations in another APOBEC deaminase.

In some embodiments, any of the fusion proteins provided herein comprise an APOBEC deaminase comprising a D316R and a D317R mutation of hAPOBEC3G (SEQ ID NO: 275), or one or more corresponding mutations in another APOBEC deaminase. In some embodiments, any of the fusion proteins provided herein comprise an APOBEC deaminase comprising a R320A mutation of hAPOBEC3G (SEQ ID NO: 275), or one or more corresponding mutations in another APOBEC deaminase. In some embodiments, any of the fusion proteins provided herein comprise an APOBEC deaminase comprising a R320E mutation of hAPOBEC3G (SEQ ID NO: 275), or one or more corresponding mutations in another APOBEC deaminase. In some embodiments, any of the fusion proteins provided herein comprise an APOBEC deaminase comprising a R313A mutation of hAPOBEC3G (SEQ ID NO: 275), or one or more corresponding mutations in another APOBEC deaminase. In some embodiments, any of the fusion proteins provided herein comprise an APOBEC deaminase comprising a W285A mutation of hAPOBEC3G (SEQ ID NO: 275), or one or more corresponding mutations in another APOBEC deaminase. In some embodiments, any of the fusion proteins provided herein comprise an APOBEC deaminase comprising a W285Y mutation of hAPOBEC3G (SEQ ID NO: 275), or one or more corresponding mutations in another APOBEC deaminase. In some embodiments, any of the fusion proteins provided herein comprise an APOBEC deaminase comprising a R326E mutation of hAPOBEC3G (SEQ ID NO: 275), or one or more corresponding mutations in another APOBEC deaminase. In some embodiments, any of the fusion proteins provided herein comprise an APOBEC deaminase comprising a W285Y and a R320E mutation of hAPOBEC3G (SEQ ID NO: 275), or one or more corresponding mutations in another APOBEC deaminase. In some embodiments, any of the fusion proteins provided herein comprise an APOBEC deaminase comprising a R320E and a R326E mutation of hAPOBEC3G (SEQ ID NO: 275), or one or more corresponding mutations in another APOBEC deaminase. In some embodiments, any of the fusion proteins provided herein comprise an APOBEC deaminase comprising a W285Y and a R326E mutation of hAPOBEC3G (SEQ ID NO: 275), or one or more corresponding mutations in another APOBEC deaminase. In some embodiments, any of the fusion proteins provided herein comprise an APOBEC deaminase comprising a W285Y, R320E, and R326E mutation of hAPOBEC3G (SEQ ID NO: 275), or one or more corresponding mutations in another APOBEC deaminase.

Some aspects of this disclosure provide fusion proteins comprising (i) a nuclease-inactive Cas9 domain; and (ii) a nucleic acid editing domain. In some embodiments, a nuclease-inactive Cas9 domain (dCas9), comprises an amino acid sequence that is at least 80%, at least 85%, at least 90%, at least 92%, at least 95%, at least 96%, at least 97%, at least 98%, at least 99%, or at least 99.5% identical to the amino acid sequence of a Cas9 as provided by any one of SEQ ID NOs: 10-263, and comprises mutations that inactivate the nuclease activity of Cas9. Mutations that render the nuclease domains of Cas9 inactive are well-known in the art. For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, the dCas9 of this disclosure comprises a D10A mutation of the amino acid sequence provided in SEQ ID NO: 10, or a corresponding mutation in any of the amino acid sequences provided in SEQ ID NOs: 11-260. In some embodiments, the dCas9 of this disclosure comprises a H840A mutation of the amino acid sequence provided in SEQ ID NO: 10, or a corresponding mutation in any of the amino acid sequences provided in SEQ ID NOs: 11-260. In some embodiments, the dCas9 of this disclosure comprises both D10A and H840A mutations of the amino acid sequence provided in SEQ ID NO: 10, or a corresponding mutation in any of the amino acid sequences provided in SEQ ID NOs: 11-260. In some embodiments, the Cas9 further comprises a histidine residue at position 840 of the amino acid sequence provided in SEQ ID NO: 10, or a corresponding mutation in any of the amino acid sequences provided in SEQ ID NOs: 11-260. The presence of the catalytic residue H840 restores the activity of the Cas9 to cleave the non-edited strand containing a G opposite the targeted C. Restoration of H840 does not result in the cleavage of the target strand containing the C. In some embodiments, the dCas9 comprises an amino acid sequence of SEQ ID NO: 263. It is to be understood that other mutations that inactivate the nuclease domains of Cas9 may also be included in the dCas9 of this disclosure.

The Cas9 or dCas9 domains comprising the mutations disclosed herein, may be a full-length Cas9, or a fragment thereof. In some embodiments, proteins comprising Cas9, or fragments thereof, are referred to as “Cas9 variants.” A Cas9 variant shares homology to Cas9, or a fragment thereof. For example a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9, e.g., a Cas9 comprising the amino acid sequence of SEQ ID NO: 10.

Any of the Cas9 fusion proteins of this disclosure may further comprise a nucleic acid editing domain (e.g., an enzyme that is capable of modifying nucleic acid, such as a deaminase). In some embodiments, the nucleic acid editing domain is a DNA-editing domain. In some embodiments, the nucleic acid editing domain has deaminase activity. In some embodiments, the nucleic acid editing domain comprises or consists of a deaminase or deaminase domain. In some embodiments, the deaminase is a cytidine deaminase. In some embodiments, the deaminase is an apolipoprotein B mRNA-editing complex (APOBEC) family deaminase. In some embodiments, the deaminase is an APOBEC1 family deaminase. In some embodiments, the deaminase is an activation-induced cytidine deaminase (AID). Some nucleic-acid editing domains as well as Cas9 fusion proteins including such domains are described in detail herein. Additional suitable nucleic acid editing domains will be apparent to the skilled artisan based on this disclosure and knowledge in the field.

Some aspects of the disclosure provide a fusion protein comprising a Cas9 domain fused to a nucleic acid editing domain, wherein the nucleic acid editing domain is fused to the N-terminus of the Cas9 domain. In some embodiments, the Cas9 domain and the nucleic acid editing-editing domain are fused via a linker. In some embodiments, the linker comprises a (GGGS)ₙ(SEQ ID NO: 265), a (GGGGS)ₙ (SEQ ID NO: 5), a (G)ₙ, an (EAAAK)ₙ (SEQ ID NO: 6), a (GGS)ₙ, (SGGS)ₙ(SEQ ID NO: 4288), an SGSETPGTSESATPES (SEQ ID NO: 7) motif (see, e.g., Guilinger J P, Thompson D B, Liu D R. Fusion of catalytically inactive Cas9 to FokI nuclease improves the specificity of genome modification. Nat. Biotechnol. 2014; 32(6): 577-82; the entire contents are incorporated herein by reference), or an (XP)ₙ motif, or a combination of any of these, wherein n is independently an integer between 1 and 30. In some embodiments, n is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or, if more than one linker or more than one linker motif is present, any combination thereof. In some embodiments, the linker comprises a (GGS)ₙ motif, wherein n is 1, 2, 3, 4, 5, 6, 7, 8, 9, 10, 11, 12, 13, 14 or 15. In some embodiments, the linker comprises a (GGS)ₙ motif, wherein n is 1, 3, or 7. In some embodiments, the linker comprises the amino acid sequence SGSETPGTSESATPES (SEQ ID NO: 7). Additional suitable linker motifs and linker configurations will be apparent to those of skill in the art. In some embodiments, suitable linker motifs and configurations include those described in Chen et al., Fusion protein linkers: property, design and functionality. Adv Drug Deliv Rev. 2013; 65(10):1357-69, the entire contents of which are incorporated herein by reference. Additional suitable linker sequences will be apparent to those of skill in the art based on the instant disclosure. In some embodiments, the general architecture of exemplary Cas9 fusion proteins provided herein comprises the structure:
[NH₂]-[nucleic acid editing domain]-[Cas9]-[COOH] or
[NH₂]-[nucleic acid editing domain]-[linker]-[Cas9]-[COOH],
   wherein NH₂ is the N-terminus of the fusion protein, and COOH is the C-terminus of the fusion protein.

The fusion proteins of the present disclosure may comprise one or more additional features. For example, in some embodiments, the fusion protein comprises a nuclear localization sequence (NLS). In some embodiments, the NLS of the fusion protein is localized between the nucleic acid editing domain and the Cas9 domain. In some embodiments, the NLS of the fusion protein is localized C-terminal to the Cas9 domain.

Other exemplary features that may be present are localization sequences, such as cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments, the nucleic acid editing domain is a deaminase. For example, in some embodiments, the general architecture of exemplary Cas9 fusion proteins with a deaminase domain comprises the structure:
[NH₂]-[NLS]-[deaminase]-[Cas9]-[COOH],
[NH₂]-[Cas9]-[deaminase]-[COOH],
[NH₂]-[deaminase]-[Cas9]-[COOH], or
[NH₂]-[deaminase]-[Cas9]-[NLS]-[COOH]
   wherein NLS is a nuclear localization sequence, NH₂ is the N-terminus of the fusion protein, and COOH is the C-terminus of the fusion protein. Nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences. In some embodiments, a NLS comprises the amino acid sequence PKKKRKV (SEQ ID NO: 741) or MDSLLMNRRKFLYQFKNVRWAKGRRETYLC (SEQ ID NO: 742). In some embodiments, a linker is inserted between the Cas9 and the deaminase. In some embodiments, the NLS is located C-terminal of the Cas9 domain. In some embodiments, the NLS is located N-terminal of the Cas9 domain. In some embodiments, the NLS is located between the deaminase and the Cas9 domain. In some embodiments, the NLS is located N-terminal of the deaminase domain. In some embodiments, the NLS is located C-terminal of the deaminase domain.

One exemplary suitable type of nucleic acid editing domain is a cytidine deaminase, for example, of the APOBEC family. The apolipoprotein B mRNA-editing complex (APOBEC) family of cytidine deaminase enzymes encompasses eleven proteins that serve to initiate mutagenesis in a controlled and beneficial manner.²⁹ One family member, activation-induced cytidine deaminase (AID), is responsible for the maturation of antibodies by converting cytosines in ssDNA to uracils in a transcription-dependent, strand-biased fashion.³⁰ The apolipoprotein B editing complex 3 (APOBEC3) enzyme provides protection to human cells against a certain HIV-1 strain via the deamination of cytosines in reverse-transcribed viral ssDNA.³¹ These proteins all require a Zn²⁺-coordinating motif (His-X-Glu-X₂₃₋₂₆-Pro-Cys-X₂₋₄-Cys; SEQ ID NO: 598) and bound water molecule for catalytic activity. The Glu residue acts to activate the water molecule to a zinc hydroxide for nucleophilic attack in the deamination reaction. Each family member preferentially deaminates at its own particular “hotspot”, ranging from WRC (W is A or T, R is A or G) for hAID, to TTC for hAPOBEC3F.³² A recent crystal structure of the catalytic domain of APOBEC3G revealed a secondary structure comprised of a five-stranded β-sheet core flanked by six α-helices, which is believed to be conserved across the entire family.³³ The active center loops have been shown to be responsible for both ssDNA binding and in determining “hotspot” identity.³⁴ Overexpression of these enzymes has been linked to genomic instability and cancer, thus highlighting the importance of sequence-specific targeting.³⁵

Some aspects of this disclosure relate to the recognition that the activity of cytidine deaminase enzymes such as APOBEC enzymes can be directed to a specific site in genomic DNA. Without wishing to be bound by any particular theory, advantages of using Cas9 as a recognition agent include (1) the sequence specificity of Cas9 can be easily altered by simply changing the sgRNA sequence; and (2) Cas9 binds to its target sequence by denaturing the dsDNA, resulting in a stretch of DNA that is single-stranded and therefore a viable substrate for the deaminase. It should be understood that other catalytic domains, or catalytic domains from other deaminases, can also be used to generate fusion proteins with Cas9, and that the disclosure is not limited in this regard.

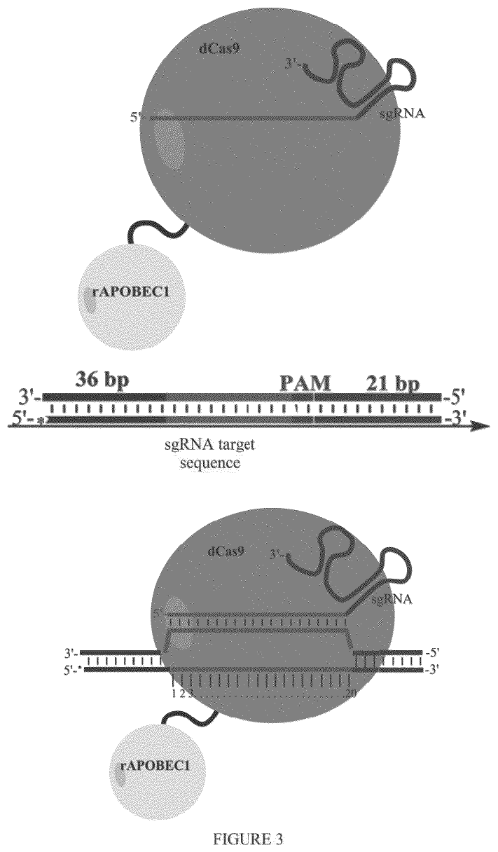

Some aspects of this disclosure are based on the recognition that Cas9:deaminase fusion proteins can efficiently deaminate nucleotides at positions 3-11 according to the numbering scheme in FIG. 3. In view of the results provided herein regarding the nucleotides that can be targeted by Cas9:deaminase fusion proteins, a person of skill in the art will be able to design suitable guide RNAs to target the fusion proteins to a target sequence that comprises a nucleotide to be deaminated.

In some embodiments, the deaminase domain and the Cas9 domain are fused to each other via a linker. Various linker lengths and flexibilities between the deaminase domain (e.g., AID) and the Cas9 domain can be employed (e.g., ranging from very flexible linkers of the form (GGGGS)ₙ (SEQ ID NO: 5), (GGS)ₙ, and (G)ₙ to more rigid linkers of the form (EAAAK)ₙ (SEQ ID NO: 6), (SGGS)ₙ(SEQ ID NO: 4288), SGSETPGTSESATPES (SEQ ID NO: 7) (see, e.g., Guilinger J P, Thompson D B, Liu D R. Fusion of catalytically inactive Cas9 to FokI nuclease improves the specificity of genome modification. Nat. Biotechnol. 2014; 32(6): 577-82; the entire contents are incorporated herein by reference) and (XP)ₙ)³⁶ in order to achieve the optimal length for deaminase activity for the specific application. In some embodiments, the linker comprises a (GGS)ₙ motif, wherein n is 1, 3, or 7. In some embodiments, the linker comprises a (an SGSETPGTSESATPES (SEQ ID NO: 7) motif.

Some exemplary suitable nucleic-acid editing domains, e.g., deaminases and deaminase domains, that can be fused to Cas9 domains according to aspects of this disclosure are provided below. It should be understood that, in some embodiments, the active domain of the respective sequence can be used, e.g., the domain without a localizing signal (nuclear localization sequence, without nuclear export signal, cytoplasmic localizing signal).

Human AID:
(SEQ ID NO: 266)
MDSLLMNRRKFLYQFKNVRWAKGRRETYLCYVVKRRDSATSFSLDFGYLRNKNGCHVELL
FLRYISDWDLDPGRCYRVTWFTSWSPCYDCARHVADFLRGNPNLSLRIFTARLYFCEDRK
AEPEGLRRLHRAGVQIAIMTFKDYFYCWNTFVENHERTFKAWEGLHENSVRLSRQLRRIL
LPLYEVDDLRDAFRTLGL
(underline: nuclear localization sequence;
double underline: nuclear export signal)
Mouse AID:
(SEQ ID NO: 267)
MDSLLMKQKKFLYHFKNVRWAKGRHETYLCYVVKRRDSATSCSLDFGHLRNKSGCHVELL
FLRYISDWDLDPGRCYRVTWFTSWSPCYDCARHVAEFLRWNPNLSLRIFTARLYFCEDRK
AEPEGLRRLHRAGVQIGIMTFKDYFYCWNTFVENRERTFKAWEGLHENSVRLTRQLRRIL
LPLYEVDDLRDAFRMLGE
(underline: nuclear localization sequence;
double underline: nuclear export signal)
Dog AID:
(SEQ ID NO: 268)
MDSLLMKQRKFLYHFKNVRWAKGRHETYLCYVVKRRDSATSFSLDFGHLRNKSGCHVELL
FLRYISDWDLDPGRCYRVTWFTSWSPCYDCARHVADFLRGYPNLSLRIFAARLYFCEDRK
AEPEGLRRLHRAGVQIAIMTFKDYFYCWNTFVENREKTFKAWEGLHENSVRLSRQLRRIL
LPLYEVDDLRDAFRTLGL
(underline: nuclear localization sequence;
double underline: nuclear export signal)
Bovine AID:
(SEQ ID NO: 269)
MDSLLKKQRQFLYQFKNVRWAKGRHETYLCYVVKRRDSPTSFSLDFGHLRNKAGCHVELL
FLRYISDWDLDPGRCYRVTWFTSWSPCYDCARHVADFLRGYPNLSLRIFTARLYFCDKER
KAEPEGLRRLHRAGVQIAIMTFKDYFYCWNTFVENHERTFKAWEGLHENSVRLSRQLRRI
LLPLYEVDDLRDAFRTLGL
(underline: nuclear localization sequence;
double underline: nuclear export signal)
Rat AID
(SEQ ID NO: 5725)
MAVGSKPKAALVGPHWERERIWCFLCSTGLGTQQTGQTSRWLRPAATQDPVSPPRSLLMK
QRKFLYHFKNVRWAKGRHETYLCYVVKRRDSATSFSLDFGYLRNKSGCHVELLFLRYISD
WDLDPGRCYRVTWFTSWSPCYDCARHVADFLRGNPNLSLRIFTARLTGWGALPAGLMSPA
RPSDYFYCWNTFVENHERTFKAWEGLHENSVRLSRRLRRILLPLYEVDDLRDAFRTLGL
(underline: nuclear localization sequence;
double underline: nuclear export signal)
Mouse APOBEC-3:
(SEQ ID NO: 270)
MGPFCLGCSHRKCYSPIRNLISQETFKFHFKNLGYAKGRKDTFLCYEVTRKDCDSPVSLH
HGVFKNKDNIHAEICFLYWFHDKVLKVLSPREEFKITWYMSWSPCFECAEQIVRFLATHH
NLSLDIFSSRLYNVQDPETQQNLCRLVQEGAQVAAMDLYEFKKCWKKFVDNGGRRFRPWK
RLLTNFRYQDSKLQEILRPCYIPVPSSSSSTLSNICLTKGLPETRFCVEGRRMDPLSEEE
FYSQFYNQRVKHLCYYHRMKPYLCYQLEQFNGQAPLKGCLLSEKGKQHAEILFLDKIRSM
ELSQVTITCYLTWSPCPNCAWQLAAFKRDRPDLILHIYTSRLYFHWKRPFQKGLCSLWQS
GILVDVMDLPQFTDCWTNFVNPKRPFWPWKGLEIISRRTQRRLRRIKESWGLQDLVNDFG
NLQLGPPMS
(italic: nucleic acid editing domain)
Rat APOBEC-3:
(SEQ ID NO: 271)
MGPFCLGCSHRKCYSPIRNLISQETFKFHFKNLRYAIDRKDTFLCYEVTRKDCDSPVSLH
HGVFKNKDNIHAEICFLYWFHDKVLKVLSPREEFKITWYMSWSPCFECAEQVLRFLATHH
NLSLDIFSSRLYNIRDPENQQNLCRLVQEGAQVAAMDLYEFKKCWKKFVDNGGRRFRPWK
KLLTNFRYQDSKLQEILRPCYIPVPSSSSSTLSNICLTKGLPETRFCVERRRVHLLSEEE
FYSQFYNQRVKHLCYYHGVKPYLCYQLEQFNGQAPLKGCLLSEKGKQHAEILFLDKIRSM
ELSQVIITCYLTWSPCPNCAWQLAAFKRDRPDLILHIYTSRLYFHWKRPFQKGLCSLWQS
GILVDVMDLPQFTDCWTNFVNPKRPFWPWKGLEIISRRTQRRLHRIKESWGLQDLVNDFG
NLQLGPPMS
(italic: nucleic acid editing domain)
Rhesus macaque APOBEC-3G:
(SEQ ID NO: 272)
MVEPMDPRTFVSNENNRPILSGLNTVWLCCEVKTKDPSGPPLDAKIFQGKVYSKAKYHPE
MRFLRWFHKWRQLHHDQEYKVTWYVSWSPCTRCANSVATFLAKDPKVTLTIFVARLYYFW
KPDYQQALRILCQKRGGPHATMKIMNYNEFQDCWNKFVDGRGKPFKPRNNLPKHYTLLQA
TLGELLRHLMDPGTFTSNFNNKPWVSGQHETYLCYKVERLHNDTWVPLNQHRGFLRNQAP
NIHGFPKGRHAELCFLDLIPFWKLDGQQYRVTCFTSWSPCFSCAQEMAKFISNNEHVSLC
IFAARIYDDQGRYQEGLRALHRDGAKIAMMNYSEFEYCWDTFVDRQGRPFQPWDGLDEHS
QALSGRLRAI
(italic: nucleic acid editing domain;
underline: cytoplasmic localization signal)
Chimpanzee APOBEC-3G:
(SEQ ID NO: 273)
MKPHFRNPVERMYQDTFSDNFYNRPILSHRNTVWLCYEVKTKGPSRPPLDAKIFRGQVYS
KLKYHPEMRFFHWFSKWRKLHRDQEYEVTWYISWSPCTKCTRDVATFLAEDPKVTLTIFV
ARLYYFWDPDYQEALRSLCQKRDGPRATMKIMNYDEFQHCWSKFVYSQRELFEPWNNLPK
YYILLHIMLGEILRHSMDPPTFTSNFNNELWVRGRHETYLCYEVERLHNDTWVLLNQRRG
FLCNQAPHKHGFLEGRHAELCFLDVIPFWKLDLHQDYRVTCFTSWSPCFSCAQEMAKFIS
NNKHVSLCIFAARIYDDQGRCQEGLRTLAKAGAKISIMTYSEFKHCWDTFVDHQGCPFQP
WDGLEEHSQALSGRLRAILQNQGN
(italic: nucleic acid editing domain;
underline: cytoplasmic localization signal)
Green monkey APOBEC-3G:
(SEQ ID NO: 274)
MNPQIRNMVEQMEPDIFVYYFNNRPILSGRNTVWLCYEVKTKDPSGPPLDANIFQGKLYP
EAKDHPEMKFLHWFRKWRQLHRDQEYEVTWYVSWSPCTRCANSVATFLAEDPKVTLTIFV
ARLYYFWKPDYQQALRILCQERGGPHATMKIMNYNEFQHCWNEFVDGQGKPFKPRKNLPK
HYTLLHATLGELLRHVMDPGTFTSNFNNKPWVSGQRETYLCYKVERSHNDTWVLLNQHRG
FLRNQAPDRHGFPKGRHAELCFLDLIPFWKLDDQQYRVTCFTSWSPCFSCAQKMAKFISN
NKHVSLCIFAARIYDDQGRCQEGLRTLHRDGAKIAVMNYSEFEYCWDTFVDRQGRPFQPW
DGLDEHSQALSGRLRAI
(italic: nucleic acid editing domain;
underline: cytoplasmic localization signal)
Human APOBEC-3G:
(SEQ ID NO: 275)
MKPHFRNTVERMYRDTFSYNFYNRPILSRRNTVWLCYEVKTKGPSRPPLDAKIFRGQVYS
ELKYHPEMRFFHWFSKWRKLHRDQEYEVTWYISWSPCTKCTRDMATFLAEDPKVTLTIFV
ARLYYFWDPDYQEALRSLCQKRDGPRATMKIMNYDEFQHCWSKFVYSQRELFEPWNNLPK
YYILLHIMLGEILRHSMDPPTFTFNFNNEPWVRGRHETYLCYEVERMHNDTWVLLNQRRG
FLCNQAPHKHGFLEGRHAELCFLDVIPFWKLDLDQDYRVTCFTSWSPCFSCAQEMAKFIS
KNKHVSLCIFTARIYDDQGRCQEGLRTLAEAGAKISIMTYSEFKHCWDTFVDHQGCPFQP
WDGLDEHSQDLSGRLRAILQNQEN
(italic: nucleic acid editing domain;
underline: cytoplasmic localization signal)
Human APOBEC-3F:
(SEQ ID NO: 276)
MKPHFRNTVERMYRDTFSYNFYNRPILSRRNTVWLCYEVKTKGPSRPRLDAKIFRGQVYS
QPEHHAEMCFLSWFCGNQLPAYKCFQITWFVSWTPCPDCVAKLAEFLAEHPNVTLTISAA
RLYYYWERDYRRALCRLSQAGARVKIMDDEEFAYCWENFVYSEGQPFMPWYKFDDNYAFL
HRTLKEILRNPMEAMYPHIFYFHFKNLRKAYGRNESWLCFTMEVVKHHSPVSWKRGVFRN
QVDPETHCHAERCFLSWFCDDILSPNTNYEVTWYTSWSPCPECAGEVAEFLARHSNVNLT
IFTARLYYFWDTDYQEGLRSLSQEGASVEIMGYKDFKYCWENFVYNDDEPFKPWKGLKYN
FLFLDSKLQEILE
(italic: nucleic acid editing domain)
Human APOBEC-3B:
(SEQ ID NO: 277)
MNPQIRNPMERMYRDTFYDNFENEPILYGRSYTWLCYEVKIKRGRSNLLWDTGVFRGQVY
FKPQYHAEMCFLSWFCGNQLPAYKCFQITWFVSWTPCPDCVAKLAEFLSEHPNVTLTISA
ARLYYYWERDYRRALCRLSQAGARVTIMDYEEFAYCWENFVYNEGQQFMPWYKFDENYAF
LHRTLKEILRYLMDPDTFTFNFNNDPLVLRRRQTYLCYEVERLDNGTWVLMDQHMGFLCN
EAKNLLCGFYGRHAELRFLDLVPSLQLDPAQIYRVTWFISWSPCFSWGCAGEVRAFLQEN
THVRLRIFAARIYDYDPLYKEALQMLRDAGAQVSIMTYDEFEYCWDTFVYRQGCPFQPWD
GLEEHSQALSGRLRAILQNQGN
(italic: nucleic acid editing domain)
Rat APOBEC-3B:
(SEQ ID NO: 5729)
MQPQGLGPNAGMGPVCLGCSHRRPYSPIRNPLKKLYQQTFYFHFKNVRYAWGRKNNFLCY
EVNGMDCALPVPLRQGVFRKQGHIHAELCFIYWFHDKVLRVLSPMEEFKVTWYMSWSPCS
KCAEQVARFLAAHRNLSLAIFSSRLYYYLRNPNYQQKLCRLIQEGVHVAAMDLPEFKKCW
NKFVDNDGQPFRPWMRLRINFSFYDCKLQEIFSRMNLLREDVFYLQFNNSHRVKPVQNRY
YRRKSYLCYQLERANGQEPLKGYLLYKKGEQHVEILFLEKMRSMELSQVRITCYLTWSPC
PNCARQLAAFKKDHPDLILRIYTSRLYFYWRKKFQKGLCTLWRSGIHVDVMDLPQFADCW
TNFVNPQRPFRPWNELEKNSWRIQRRLRRIKESWGL
Bovine APOBEC-3B:
(SEQ ID NO: 5730)
DGWEVAFRSGTVLKAGVLGVSMTEGWAGSGHPGQGACVWTPGTRNTMNLLREVLFKQQFG
NQPRVPAPYYRRKTYLCYQLKQRNDLTLDRGCFRNKKQRHAEIRFIDKINSLDLNPSQSY
KIICYITWSPCPNCANELVNFITRNNHLKLEIFASRLYFHWIKSFKMGLQDLQNAGISVA
VMTHTEFEDCWEQFVDNQSRPFQPWDKLEQYSASIRRRLQRILTAPI
Chimpanzee APOBEC-3B:
(SEQ ID NO: 5731)
MNPQIRNPMEWMYQRTFYYNFENEPILYGRSYTWLCYEVKIRRGHSNLLWDTGVFRGQMY
SQPEHHAEMCFLSWFCGNQLSAYKCFQITWFVSWTPCPDCVAKLAKFLAEHPNVTLTISA
ARLYYYWERDYRRALCRLSQAGARVKIMDDEEFAYCWENFVYNEGQPFMPWYKFDDNYAF
LHRTLKEIIRHLMDPDTFTFNFNNDPLVLRRHQTYLCYEVERLDNGTWVLMDQHMGFLCN
EAKNLLCGFYGRHAELRFLDLVPSLQLDPAQIYRVTWFISWSPCFSWGCAGQVRAFLQEN
THVRLRIFAARIYDYDPLYKEALQMLRDAGAQVSIMTYDEFEYCWDTFVYRQGCPFQPWD
GLEEHSQALSGRLRAILQVRASSLCMVPHRPPPPPQSPGPCLPLCSEPPLGSLLPTGRPA
PSLPFLLTASFSFPPPASLPPLPSLSLSPGHLPVPSFHSLTSCSIQPPCSSRIRETEGWA
SVSKEGRDLG
Human APOBEC-3C:
(SEQ ID NO: 278)
MNPQIRNPMKAMYPGTFYFQFKNLWEANDRNETWLCFTVEGIKRRSVVSWKTGVFRNQVD
SETHCHAERCFLSWFCDDILSPNTKYQVTWYTSWSPCPDCAGEVAEFLARHSNVNLTIFT
ARLYYFQYPCYQEGLRSLSQEGVAVEIMDYEDFKYCWENFVYNDNEPFKPWKGLKTNFRL
LKRRLRESLQ
(italic: nucleic acid editing domain)
Gorilla APOBEC3C
(SEQ ID NO: 5726)
MNPQIRNPMKAMYPGTFYFQFKNLWEANDRNETWLCFTVEGIKRRSVVSWKTGVFRNQVD
SETHCHAERCFLSWFCDDILSPNTNYQVTWYTSWSPCPECAGEVAEFLARHSNVNLTIFT
ARLYYFQDTDYQEGLRSLSQEGVAVKIMDYKDFKYCWENFVYNDDEPFKPWKGLKYNFRF
LKRRLQEILE
(italic: nucleic acid editing domain)
Human APOBEC-3A:
(SEQ ID NO: 279)
MEASPASGPRHLMDPHIFTSNFNNGIGRHKTYLCYEVERLDNGTSVKMDQHRGFLHNQAK
NLLCGFYGRHAELRFLDLVPSLQLDPAQIYRVTWFISWSPCFSWGCAGEVRAFLQENTHV
RLRIFAARIYDYDPLYKEALQMLRDAGAQVSIMTYDEFKHCWDTFVDHQGCPFQPWDGLD
EHSQALSGRLRAILQNQGN
(italic: nucleic acid editing domain)
Rhesus macaque APOBEC-3A:
(SEQ ID NO: 5727)
MDGSPASRPRHLMDPNTFTFNFNNDLSVRGRHQTYLCYEVERLDNGTWVPMDERRGFLCN
KAKNVPCGDYGCHVELRFLCEVPSWQLDPAQTYRVTWFISWSPCFRRGCAGQVRVFLQEN
KHVRLRIFAARIYDYDPLYQEALRTLRDAGAQVSIMTYEEFKHCWDTFVDRQGRPFQPWD
GLDEHSQALSGRLRAILQNQGN
(italic: nucleic acid editing domain)
Bovine APOBEC-3A:
(SEQ ID NO: 5728)
MDEYTFTENFNNQGWPSKTYLCYEMERLDGDATIPLDEYKGFVRNKGLDQPEKPCHAELY
FLGKIHSWNLDRNQHYRLTCFISWSPCYDCAQKLTTFLKENHHISLHILASRIYTHNRFG
CHQSGLCELQAAGARITIMTFEDFKHCWETFVDHKGKPFQPWEGLNVKSQALCTELQAIL
KTQQN
(italic: nucleic acid editing domain)
Human APOBEC-3H:
(SEQ ID NO: 280)
MALLTAETFRLQFNNKRRLRRPYYPRKALLCYQLTPQNGSTPTRGYFENKKKCHAEICFI
NEIKSMGLDETQCYQVTCYLTWSPCSSCAWELVDFIKAHDHLNLGIFASRLYYHWCKPQQ
KGLRLLCGSQVPVEVMGFPKFADCWENFVDHEKPLSFNPYKMLEELDKNSRAIKRRLERI
KIPGVRAQGRYMDILCDAEV
(italic: nucleic acid editing domain)
Rhesus macaque APOBEC-3H:
(SEQ ID NO: 5732)
MALLTAKTFSLQFNNKRRVNKPYYPRKALLCYQLTPQNGSTPTRGHLKNKKKDHAEIRFI
NKIKSMGLDETQCYQVTCYLTWSPCPSCAGELVDFIKAHRHLNLRIFASRLYYHWRPNYQ
EGLLLLCGSQVPVEVMGLPEFTDCWENFVDHKEPPSFNPSEKLEELDKNSQAIKRRLERI
KSRSVDVLENGLRSLQLGPVTPSSSIRNSR
Human APOBEC-3D:
(SEQ ID NO: 281)
MNPQIRNPMERMYRDTFYDNFENEPILYGRSYTWLCYEVKIKRGRSNLLWDTGVFRGPVL
PKRQSNHRQEVYFRFENHAEMCFLSWFCGNRLPANRRFQITWFVSWNPCLPCVVKVTKFL
AEHPNVTLTISAARLYYYRDRDWRWVLLRLHKAGARVKIMDYEDFAYCWENFVCNEGQPF
MPWYKFDDNYASLHRTLKEILRNPMEAMYPHIFYFHFKNLLKACGRNESWLCFTMEVTKH
HSAVFRKRGVFRNQVDPETHCHAERCFLSWFCDDILSPNTNYEVTWYTSWSPCPECAGEV
AEFLARHSNVNLTIFTARLCYFWDTDYQEGLCSLSQEGASVKIMGYKDFVSCWKNFVYSD
DEPFKPWKGLQTNFRLLKRRLREILQ
(italic: nucleic acid editing domain)
Human APOBEC-1:
(SEQ ID NO: 282)
MTSEKGPSTGDPTLRRRIEPWEFDVFYDPRELRKEACLLYEIKWGMSRKIWRSSGKNTTN
HVEVNFIKKFTSERDFHPSMSCSITWFLSWSPCWECSQAIREFLSRHPGVTLVIYVARLF
WHMDQQNRQGLRDLVNSGVTIQIMRASEYYHCWRNFVNYPPGDEAHWPQYPPLWMMLYAL
ELHCIILSLPPCLKISRRWQNHLTFFRLHLQNCHYQTIPPHILLATGLIHPSVAWR
Mouse APOBEC-1:
(SEQ ID NO: 283)
MSSETGPVAVDPTLRRRIEPHEFEVFFDPRELRKETCLLYEINWGGRHSVWRHTSQNTSN
HVEVNFLEKFTTERYFRPNTRCSITWFLSWSPCGECSRAITEFLSRHPYVTLFIYIARLY
HHTDQRNRQGLRDLISSGVTIQIMTEQEYCYCWRNFVNYPPSNEAYWPRYPHLWVKLYVL
ELYCIILGLPPCLKILRRKQPQLTFFTITLQTCHYQRIPPHLLWATGLK
Rat APOBEC-1:
(SEQ ID NO: 284)
MSSETGPVAVDPTLRRRIEPHEFEVFFDPRELRKETCLLYEINWGGRHSIWRHTSQNTNK
HVEVNFIEKFTTERYFCPNTRCSITWFLSWSPCGECSRAITEFLSRYPHVTLFIYIARLY
HHADPRNRQGLRDLISSGVTIQIMTEQESGYCWRNFVNYSPSNEAHWPRYPHLWVRLYVL
ELYCIILGLPPCLNILRRKQPQLTFFTIALQSCHYQRLPPHILWATGLK
Human APOBEC-2:
(SEQ ID NO: 5733)
MAQKEEAAVATEAASQNGEDLENLDDPEKLKELIELPPFEIVTGERLPANFFKFQFRNVE
YSSGRNKTFLCYVVEAQGKGGQVQASRGYLEDEHAAAHAEEAFFNTILPAFDPALRYNVT
WYVSSSPCAACADRIIKTLSKTKNLRLLILVGRLFMWEEPEIQAALKKLKEAGCKLRIMK
PQDFEYVWQNFVEQEEGESKAFQPWEDIQENFLYYEEKLADILK
Mouse APOBEC-2:
(SEQ ID NO: 5734)
MAQKEEAAEAAAPASQNGDDLENLEDPEKLKELIDLPPFEIVTGVRLPVNFFKFQFRNVE
YSSGRNKTFLCYVVEVQSKGGQAQATQGYLEDEHAGAHAEEAFFNTILPAFDPALKYNVT
WYVSSSPCAACADRILKTLSKTKNLRLLILVSRLFMWEEPEVQAALKKLKEAGCKLRIMK
PQDFEYIWQNFVEQEEGESKAFEPWEDIQENFLYYEEKLADILK
Rat APOBEC-2:
(SEQ ID NO: 5735)
MAQKEEAAEAAAPASQNGDDLENLEDPEKLKELIDLPPFEIVTGVRLPVNFFKFQFRNVE
YSSGRNKTFLCYVVEAQSKGGQVQATQGYLEDEHAGAHAEEAFFNTILPAFDPALKYNVT
WYVSSSPCAACADRILKTLSKTKNLRLLILVSRLFMWEEPEVQAALKKLKEAGCKLRIMK
PQDFEYLWQNFVEQEEGESKAFEPWEDIQENFLYYEEKLADILK
Bovine APOBEC-2:
(SEQ ID NO: 5736)
MAQKEEAAAAAEPASQNGEEVENLEDPEKLKELIELPPFEIVTGERLPAHYFKFQFRNVE
YSSGRNKTFLCYVVEAQSKGGQVQASRGYLEDEHATNHAEEAFFNSIMPTFDPALRYMVT
WYVSSSPCAACADRIVKTLNKTKNLRLLILVGRLFMWEEPEIQAALRKLKEAGCRLRIMK
PQDFEYIWQNFVEQEEGESKAFEPWEDIQENFLYYEEKLADILK
Petromyzon marinus CDA1 (pmCDA1)
(SEQ ID NO: 5738)
MTDAEYVRIHEKLDIYTFKKQFFNNKKSVSHRCYVLFELKRRGERRACFWGYAVNKPQSG
TERGIHAEIFSIRKVEEYLRDNPGQFTINWYSSWSPCADCAEKILEWYNQELRGNGHTLK
IWACKLYYEKNARNQIGLWNLRDNGVGLNVMVSEHYQCCRKIFIQSSHNQLNENRWLEKT
LKRAEKRRSELSIMIQVKILHTTKSPAV
Human APOBEC3G D316R_D317R
(SEQ ID NO: 5739)
MKPHFRNTVERMYRDTFSYNFYNRPILSRRNTVWLCYEVKTKGPSRPPLDAKIFRGQVYS
ELKYHPEMRFFHWFSKWRKLHRDQEYEVTWYISWSPCTKCTRDMATFLAEDPKVTLTIFV
ARLYYFWDPDYQEALRSLCQKRDGPRATMKIMNYDEFQHCWSKFVYSQRELFEPWNNLPK
YYILLHIMLGEILRHSMDPPTFTFNFNNEPWVRGRHETYLCYEVERMHNDTWVLLNQRRG
FLCNQAPHKHGFLEGRHAELCFLDVIPFWKLDLDQDYRVTCFTSWSPCFSCAQEMAKFIS
KNKHVSLCIFTARIYRRQGRCQEGLRTLAEAGAKISIMTYSEFKHCWDTFVDHQGCPFQP
WDGLDEHSQDLSGRLRAILQNQEN
Human APOBEC3G chain A
(SEQ ID NO: 5740)
MDPPTFTFNFNNEPWVRGRHETYLCYEVERMHNDTWVLLNQRRGFLCNQAPHKHGFLEGR
HAELCFLDVIPFWKLDLDQDYRVTCFTSWSPCFSCAQEMAKFISKNKHVSLCIFTARIYD
DQGRCQEGLRTLAEAGAKISIMTYSEFKHCWDTFVDHQGCPFQPWDGLDEHSQDLSGRLR
AILQ
Human APOBEC3G chain A D120R_D121R
(SEQ ID NO: 5741)
MDPPTFTFNFNNEPWVRGRHETYLCYEVERMHNDTWVLLNQRRGFLCNQAPHKHGFLEGR
HAELCFLDVIPFWKLDLDQDYRVTCFTSWSPCFSCAQEMAKFISKNKHVSLCIFTARIYR
RQGRCQEGLRTLAEAGAKISIMTYSEFKHCWDTFVDHQGCPFQPWDGLDEHSQDLSGRLR
AILQ

In some embodiments, fusion proteins as provided herein comprise the full-length amino acid of a nucleic acid editing enzyme, e.g., one of the sequences provided above. In other embodiments, however, fusion proteins as provided herein do not comprise a full-length sequence of a nucleic acid editing enzyme, but only a fragment thereof. For example, in some embodiments, a fusion protein provided herein comprises a Cas9 domain and a fragment of a nucleic acid editing enzyme, e.g., wherein the fragment comprises a nucleic acid editing domain. Exemplary amino acid sequences of nucleic acid editing domains are shown in the sequences above as italicized letters, and additional suitable sequences of such domains will be apparent to those of skill in the art.

Additional suitable nucleic-acid editing enzyme sequences, e.g., deaminase enzyme and domain sequences, that can be used according to aspects of this invention, e.g., that can be fused to a nuclease-inactive Cas9 domain, will be apparent to those of skill in the art based on this disclosure. In some embodiments, such additional enzyme sequences include deaminase enzyme or deaminase domain sequences that are at least 70%, at least 75%, at least 80%, at least 85%, at least 90%, at least 95%, at least 96%, at least 97%, at least 98%, or at least 99% similar to the sequences provided herein. Additional suitable Cas9 domains, variants, and sequences will also be apparent to those of skill in the art. Examples of such additional suitable Cas9 domains include, but are not limited to, D10A, D10A/D839A/H840A, and D10A/D839A/H840A/N863A mutant domains (See, e.g., Prashant et al., CAS9 transcriptional activators for target specificity screening and paired nickases for cooperative genome engineering. Nature Biotechnology. 2013; 31(9): 833-838 the entire contents of which are incorporated herein by reference). In some embodiments, the Cas9 comprises a histidine residue at position 840 of the amino acid sequence provided in SEQ ID NO: 10, or a corresponding mutation in any of the amino acid sequences provided in SEQ ID NOs: 11-260. The presence of the catalytic residue H840 restores the activity of the Cas9 to cleave the non-edited strand containing a G opposite the targeted C. Restoration of H840 does not result in the cleavage of the target strand containing the C.

Additional suitable strategies for generating fusion proteins comprising a Cas9 domain and a deaminase domain will be apparent to those of skill in the art based on this disclosure in combination with the general knowledge in the art. Suitable strategies for generating fusion proteins according to aspects of this disclosure using linkers or without the use of linkers will also be apparent to those of skill in the art in view of the instant disclosure and the knowledge in the art. For example, Gilbert et al., CRISPR-mediated modular RNA-guided regulation of transcription in eukaryotes. Cell. 2013; 154(2):442-51, showed that C-terminal fusions of Cas9 with VP64 using 2 NLS's as a linker (SPKKKRKVEAS, SEQ ID NO: 599), can be employed for transcriptional activation. Mali et al., CAS9 transcriptional activators for target specificity screening and paired nickases for cooperative genome engineering. Nat Biotechnol. 2013; 31(9):833-8, reported that C-terminal fusions with VP64 without linker can be employed for transcriptional activation. And Maeder et al., CRISPR RNA-guided activation of endogenous human genes. Nat Methods. 2013; 10: 977-979, reported that C-terminal fusions with VP64 using a Gly4Ser (SEQ ID NO: 5) linker can be used as transcriptional activators. Recently, dCas9-FokI nuclease fusions have successfully been generated and exhibit improved enzymatic specificity as compared to the parental Cas9 enzyme (In Guilinger J P, Thompson D B, Liu D R. Fusion of catalytically inactive Cas9 to FokI nuclease improves the specificity of genome modification. Nat. Biotechnol. 2014; 32(6): 577-82, and in Tsai S Q, Wyvekens N, Khayter C, Foden J A, Thapar V, Reyon D, Goodwin M J, Aryee M J, Joung J K. Dimeric CRISPR RNA-guided FokI nucleases for highly specific genome editing. Nat Biotechnol. 2014; 32(6):569-76. PMID: 24770325 a SGSETPGTSESATPES (SEQ ID NO: 7) or a GGGGS (SEQ ID NO: 5) linker was used in FokI-dCas9 fusion proteins, respectively).

Some aspects of this disclosure provide fusion proteins comprising (i) a Cas9 enzyme or domain (e.g., a first protein); and (ii) a nucleic acid-editing enzyme or domain (e.g., a second protein). In some aspects, the fusion proteins provided herein further include (iii) a programmable DNA-binding protein, for example, a zinc-finger domain, a TALE, or a second Cas9 protein (e.g., a third protein). Without wishing to be bound by any particular theory, fusing a programmable DNA-binding protein (e.g., a second Cas9 protein) to a fusion protein comprising (i) a Cas9 enzyme or domain (e.g., a first protein); and (ii) a nucleic acid-editing enzyme or domain (e.g., a second protein) may be useful for improving specificity of the fusion protein to a target nucleic acid sequence, or for improving specificity or binding affinity of the fusion protein to bind target nucleic acid sequence that does not contain a canonical PAM (NGG) sequence. In some embodiments, the third protein is a Cas9 protein (e.g, a second Cas9 protein). In some embodiments, the third protein is any of the Cas9 proteins provided herein. In some embodiments, the third protein is fused to the fusion protein N-terminal to the Cas9 protein (e.g., the first protein). In some embodiments, the third protein is fused to the fusion protein C-terminal to the Cas9 protein (e.g., the first protein). In some embodiments, the Cas9 domain (e.g., the first protein) and the third protein (e.g., a second Cas9 protein) are fused via a linker (e.g., a second linker). In some embodiments, the linker comprises a (GGGGS)ₙ (SEQ ID NO: 5), a (G)ₙ, an (EAAAK)ₙ (SEQ ID NO: 6), a (GGS)ₙ, (SGGS)ₙ (SEQ ID NO: 4288), an SGSETPGTSESATPES (SEQ ID NO: 7), or an (XP)ₙ motif, or a combination of any of these, wherein n is independently an integer between 1 and 30. In some embodiments, the general architecture of exemplary Cas9 fusion proteins provided herein comprises the structure:
[NH2]-[nucleic acid-editing enzyme or domain]-[Cas9]-[third protein]-[COOH];
[NH2]-[third protein]-[Cas9]-[nucleic acid-editing enzyme or domain]-[COOH];
[NH2]-[Cas9]-[nucleic acid-editing enzyme or domain]-[third protein]-[COOH];
[NH2]-[third protein]-[nucleic acid-editing enzyme or domain]-[Cas9]-[COOH];
[NH2]-[UGI]-[nucleic acid-editing enzyme or domain]-[Cas9]-[third protein]-[COOH];
[NH2]-[UGI]-[third protein]-[Cas9]-[nucleic acid-editing enzyme or domain]-[COOH];
[NH2]-[UGI]-[Cas9]-[nucleic acid-editing enzyme or domain]-[third protein]-[COOH];
[NH2]-[UGI]-[third protein]-[nucleic acid-editing enzyme or domain]-[Cas9]-[COOH];
[NH2]-[nucleic acid-editing enzyme or domain]-[Cas9]-[third protein]-[UGI]-[COOH];
[NH2]-[third protein]-[Cas9]-[nucleic acid-editing enzyme or domain]-[UGI]-[COOH];
[NH2]-[Cas9]-[nucleic acid-editing enzyme or domain]-[third protein]-[UGI]-[COOH]; or
[NH2]-[third protein]-[nucleic acid-editing enzyme or domain]-[Cas9]-[UGI]-[COOH];
   wherein NH2 is the N-terminus of the fusion protein, and COOH is the C-terminus of the fusion protein. In some embodiments, the “]-[” used in the general architecture above indicates the presence of an optional linker sequence. In other examples, the general architecture of exemplary Cas9 fusion proteins provided herein comprises the structure:
[NH2]-[nucleic acid-editing enzyme or domain]-[Cas9]-[second Cas9 protein]-[COOH];
[NH2]-[second Cas9 protein]-[Cas9]-[nucleic acid-editing enzyme or domain]-[COOH];
[NH2]-[Cas9]-[nucleic acid-editing enzyme or domain]-[second Cas9 protein]-[COOH];
[NH2]-[second Cas9 protein]-[nucleic acid-editing enzyme or domain]-[Cas9]-[COOH];
[NH2]-[UGI]-[nucleic acid-editing enzyme or domain]-[Cas9]-[second Cas9 protein]-[COOH],
[NH2]-[UGI]-[second Cas9 protein]-[Cas9]-[nucleic acid-editing enzyme or domain]-[COOH];
[NH2]-[UGI]-[Cas9]-[nucleic acid-editing enzyme or domain]-[second Cas9 protein]-[COOH];
[NH2]-[UGI]-[second Cas9 protein]-[nucleic acid-editing enzyme or domain]-[Cas9]-[COOH];
[NH2]-[nucleic acid-editing enzyme or domain]-[Cas9]-[second Cas9 protein]-[UGI]-[COOH];
[NH2]-[second Cas9 protein]-[Cas9]-[nucleic acid-editing enzyme or domain]-[UGI]-[COOH];
[NH2]-[Cas9]-[nucleic acid-editing enzyme or domain]-[second Cas9 protein]-[UGI]-[COOH]; or
[NH2]-[second Cas9 protein]-[nucleic acid-editing enzyme or domain]-[Cas9]-[UGI]-[COOH];
   wherein NH₂ is the N-terminus of the fusion protein, and COOH is the C-terminus of the fusion protein. In some embodiments, the “]-[” used in the general architecture above indicates the presence of an optional linker sequence. In some embodiments, the second Cas9 is a dCas9 protein. In some examples, the general architecture of exemplary Cas9 fusion proteins provided herein comprises a structure as shown in FIG. 3. It should be appreciated that any of the proteins provided in any of the general architectures of exemplary Cas9 fusion proteins may be connected by one or more of the linkers provided herein. In some embodiments, the linkers are the same. In some embodiments, the linkers are different. In some embodiments, one or more of the proteins provided in any of the general architectures of exemplary Cas9 fusion proteins are not fused via a linker. In some embodiments, the fusion proteins further comprise a nuclear targeting sequence, for example a nuclear localization sequence. In some embodiments, fusion proteins provided herein further comprise a nuclear localization sequence (NLS). In some embodiments, the NLS is fused to the N-terminus of the fusion protein. In some embodiments, the NLS is fused to the C-terminus of the fusion protein. In some embodiments, the NLS is fused to the N-terminus of the third protein. In some embodiments, the NLS is fused to the C-terminus of the third protein. In some embodiments, the NLS is fused to the N-terminus of the Cas9 protein. In some embodiments, the NLS is fused to the C-terminus of the Cas9 protein. In some embodiments, the NLS is fused to the N-terminus of the nucleic acid-editing enzyme or domain. In some embodiments, the NLS is fused to the C-terminus of the nucleic acid-editing enzyme or domain. In some embodiments, the NLS is fused to the N-terminus of the UGI protein. In some embodiments, the NLS is fused to the C-terminus of the UGI protein. In some embodiments, the NLS is fused to the fusion protein via one or more linkers. In some embodiments, the NLS is fused to the fusion protein without a linker

### Uracil Glycosylase Inhibitor Fusion Proteins

Some aspects of the disclosure relate to fusion proteins that comprise a uracil glycosylase inhibitor (UGI) domain. In some embodiments, any of the fusion proteins provided herein that comprise a Cas9 domain (e.g., a nuclease active Cas9 domain, a nuclease inactive dCas9 domain, or a Cas9 nickase) may be further fused to a UGI domain either directly or via a linker. Some aspects of this disclosure provide deaminase-dCas9 fusion proteins, deaminase-nuclease active Cas9 fusion proteins and deaminase-Cas9 nickase fusion proteins with increased nucleobase editing efficiency. Without wishing to be bound by any particular theory, cellular DNA-repair response to the presence of U:G heteroduplex DNA may be responsible for the decrease in nucleobase editing efficiency in cells. For example, uracil DNA glycosylase (UDG) catalyzes removal of U from DNA in cells, which may initiate base excision repair, with reversion of the U:G pair to a C:G pair as the most common outcome. As demonstrated in the Examples below, Uracil DNA Glycosylase Inhibitor (UGI) may inhibit human UDG activity. Thus, this disclosure contemplates a fusion protein comprising dCas9-nucleic acid editing domain further fused to a UGI domain. This disclosure also contemplates a fusion protein comprising a Cas9 nickase-nucleic acid editing domain further fused to a UGI domain. It should be understood that the use of a UGI domain may increase the editing efficiency of a nucleic acid editing domain that is capable of catalyzing a C to U change. For example, fusion proteins comprising a UGI domain may be more efficient in deaminating C residues. In some embodiments, the fusion protein comprises the structure:
[deaminase]-[optional linker sequence]-[dCas9]-[optional linker sequence]-[UGI];
[deaminase]-[optional linker sequence]-[UGI]-[optional linker sequence]-[dCas9];
[UGI]-[optional linker sequence]-[deaminase]-[optional linker sequence]-[dCas9];
[UGI]-[optional linker sequence]-[dCas9]-[optional linker sequence]-[deaminase];
[dCas9]-[optional linker sequence]-[deaminase]-[optional linker sequence]-[UGI]; or
[dCas9]-[optional linker sequence]-[UGI]-[optional linker sequence]-[deaminase].
   In other embodiments, the fusion protein comprises the structure:
[deaminase]-[optional linker sequence]-[Cas9 nickase]-[optional linker sequence]-[UGI];
[deaminase]-[optional linker sequence]-[UGI]-[optional linker sequence]-[Cas9 nickase];
[UGI]-[optional linker sequence]-[deaminase]-[optional linker sequence]-[Cas9 nickase];
[UGI]-[optional linker sequence]-[Cas9 nickase]-[optional linker sequence]-[deaminase];
[Cas9 nickase]-[optional linker sequence]-[deaminase]-[optional linker sequence]-[UGI]; or
[Cas9 nickase]-[optional linker sequence]-[UGI]-[optional linker sequence]-[deaminase].

In some embodiments, the fusion proteins provided herein do not comprise a linker sequence. In some embodiments, one or both of the optional linker sequences are present.

In some embodiments, the “-” used in the general architecture above indicates the presence of an optional linker sequence. In some embodiments, the fusion proteins comprising a UGI further comprise a nuclear targeting sequence, for example a nuclear localization sequence. In some embodiments, fusion proteins provided herein further comprise a nuclear localization sequence (NLS). In some embodiments, the NLS is fused to the N-terminus of the fusion protein. In some embodiments, the NLS is fused to the C-terminus of the fusion protein. In some embodiments, the NLS is fused to the N-terminus of the UGI protein. In some embodiments, the NLS is fused to the C-terminus of the UGI protein. In some embodiments, the NLS is fused to the N-terminus of the Cas9 protein. In some embodiments, the NLS is fused to the C-terminus of the Cas9 protein. In some embodiments, the NLS is fused to the N-terminus of the deaminase. In some embodiments, the NLS is fused to the C-terminus of the deaminase. In some embodiments, the NLS is fused to the N-terminus of the second Cas9. In some embodiments, the NLS is fused to the C-terminus of the second Cas9. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. In some embodiments, the NLS comprises an amino acid sequence as set forth in SEQ ID NO: 741 or SEQ ID NO: 742.

In some embodiments, a UGI domain comprises a wild-type UGI or a UGI as set forth in SEQ ID NO: 600. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 600. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 600. In some embodiments, a UGI comprises an amino acid sequence homologous to the amino acid sequence set forth in SEQ ID NO: 600 or an amino acid sequence homologous to a fragment of the amino acid sequence set forth in SEQ ID NO: 600. In some embodiments, proteins comprising UGI or fragments of UGI or homologs of UGI or UGI fragments are referred to as “UGI variants.” A UGI variant shares homology to UGI, or a fragment thereof. For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 600. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 600. In some embodiments, the UGI comprises the following amino acid sequence:

>sp P14739|UNGI_BPPB2 Uracil-DNA glycosylase
inhibitor
(SEQ ID NO: 600)
MTNLSDIIEKETGKQLVIQESILMLPEEVEEVIGNKPESDIL
VHTAYDESTDENVMLLTSDAPEYKPWALVIQDSNGENKIKML

Suitable UGI protein and nucleotide sequences are provided herein and additional suitable UGI sequences are known to those in the art, and include, for example, those published in Wang et al., Uracil-DNA glycosylase inhibitor gene of bacteriophage PBS2 encodes a binding protein specific for uracil-DNA glycosylase. J. Biol. Chem. 264:1163-1171(1989); Lundquist et al., Site-directed mutagenesis and characterization of uracil-DNA glycosylase inhibitor protein. Role of specific carboxylic amino acids in complex formation with Escherichia coli uracil-DNA glycosylase. J. Biol. Chem. 272:21408-21419(1997); Ravishankar et al., X-ray analysis of a complex of Escherichia coli uracil DNA glycosylase (EcUDG) with a proteinaceous inhibitor. The structure elucidation of a prokaryotic UDG. Nucleic Acids Res. 26:4880-4887(1998); and Putnam et al., Protein mimicry of DNA from crystal structures of the uracil-DNA glycosylase inhibitor protein and its complex with Escherichia coli uracil-DNA glycosylase. J. Mol. Biol. 287:331-346(1999), the entire contents of each are incorporated herein by reference.

It should be appreciated that additional proteins may be uracil glycosylase inhibitors. For example, other proteins that are capable of inhibiting (e.g., sterically blocking) a uracil-DNA glycosylase base-excision repair enzyme are within the scope of this disclosure. Additionally, any proteins that block or inhibit base-excision repair as also within the scope of this disclosure. In some embodiments, a protein that binds DNA is used. In another embodiment, a substitute for UGI is used. In some embodiments, a uracil glycosylase inhibitor is a protein that binds single-stranded DNA. For example, a uracil glycosylase inhibitor may be a Erwinia tasmaniensis single-stranded binding protein. In some embodiments, the single-stranded binding protein comprises the amino acid sequence (SEQ ID NO: 322). In some embodiments, a uracil glycosylase inhibitor is a protein that binds uracil. In some embodiments, a uracil glycosylase inhibitor is a protein that binds uracil in DNA. In some embodiments, a uracil glycosylase inhibitor is a catalytically inactive uracil DNA-glycosylase protein. In some embodiments, a uracil glycosylase inhibitor is a catalytically inactive uracil DNA-glycosylase protein that does not excise uracil from the DNA. For example, a uracil glycosylase inhibitor is a UdgX. In some embodiments, the UdgX comprises the amino acid sequence (SEQ ID NO: 323). As another example, a uracil glycosylase inhibitor is a catalytically inactive UDG. In some embodiments, a catalytically inactive UDG comprises the amino acid sequence (SEQ ID NO: 324). It should be appreciated that other uracil glycosylase inhibitors would be apparent to the skilled artisan and are within the scope of this disclosure. In some embodiments, a uracil glycosylase inhibitor is a protein that is homologous to any one of SEQ ID NOs: 322-324. In some embodiments, a uracil glycosylase inhibitor is a protein that is at least 50% identical, at least 55% identical at least 60% identical, at least 65% identical, at least 70% identical, at least 75% identical, at least 80% identical at least 85% identical, at least 90% identical, at least 95% identical, at least 96% identical, at least 98% identical, at least 99% identical, or at least 99.5% identical to any one of SEQ ID NOs: 322-324.

Erwinia tasmaniensis SSB (themostable single-
stranded DNA binding protein)
(SEQ ID NO: 322)
MASRGVNKVILVGNLGQDPEVRYMPNGGAVANITLATSESWRDKQ
TGETKEKTEWHRVVLFGKLAEVAGEYLRKGSQVYIEGALQTRKWT
DQAGVEKYTTEVVVNVGGTMQMLGGRSQGGGASAGGONGGSNNGW
GQPQQPQGGNQFSGGAQQQARPQQQPQQNNAPANNEPPIDFDDDI
P
UdgX (binds to Uracil in DNA but does not
excise)
(SEQ ID NO: 323)
MAGAQDFVPHTADLAELAAAAGECRGCGLYRDATQAVFGAGGRSA
RIMMIGEQPGDKEDLAGLPFVGPAGRLLDRALEAADIDRDALYVT
NAVKHFKFTRAAGGKRRIHKTPSRTEVVACRPWLIAEMTSVEPDV
VVLLGATAAKALLGNDFRVTQHRGEVLHVDDVPGDPALVATVHPS
SLLRGPKEERESAFAGLVDDLRVAADVRP
UDG (catalytically inactive human UDG, binds
to Uracil in DNA but does not excise)
(SEQ ID NO: 324)
MIGQKTLYSFFSPSPARKRHAPSPEPAVQGTGVAGVPEESGDAAA
IPAKKAPAGQEEPGTPPSSPLSAEQLDRIQRNKAAALLRLAARNV
PVGFGESWKKHLSGEFGKPYFIKLMGFVAEERKHYTVYPPPHQVF
TWTQMCDIKDVKVVILGQEPYHGPNQAHGLCFSVQRPVPPPPSLE
NIYKELSTDIEDFVHPGHGDLSGWAKQGVLLLNAVLTVRAHQANS
HKERGWEQFTDAVVSWLNQNSNGLVFLLWGSYAQKKGSAIDRKRH
HVLQTAHPSPLSVYRGFFGCRHFSKTNELLQKSGKKPIDWKEL

In some embodiments, the nucleic acid editing domain is a deaminase domain. In some embodiments, the deaminase is a cytosine deaminase or a cytidine deaminase. In some embodiments, the deaminase is an apolipoprotein B mRNA-editing complex (APOBEC) family deaminase. In some embodiments, the deaminase is an APOBEC1 deaminase. In some embodiments, the deaminase is an APOBEC2 deaminase. In some embodiments, the deaminase is an APOBEC3 deaminase. In some embodiments, the deaminase is an APOBEC3A deaminase. In some embodiments, the deaminase is an APOBEC3B deaminase. In some embodiments, the deaminase is an APOBEC3C deaminase. In some embodiments, the deaminase is an APOBEC3D deaminase. In some embodiments, the deaminase is an APOBEC3E deaminase. In some embodiments, the deaminase is an APOBEC3F deaminase. In some embodiments, the deaminase is an APOBEC3G deaminase. In some embodiments, the deaminase is an APOBEC3H deaminase. In some embodiments, the deaminase is an APOBEC4 deaminase. In some embodiments, the deaminase is an activation-induced deaminase (AID). In some embodiments, the deminase is a rat APOBEC1 (SEQ ID NO: 282). In some embodiments, the deminase is a human APOBEC1 (SEQ ID No: 284). In some embodiments, the deaminase is a Petromyzon marinus cytidine deaminase 1 (pmCDA1). In some embodiments, the deminase is a human APOBEC3G (SEQ ID NO: 275). In some embodiments, the deaminase is a fragment of the human APOBEC3G (SEQ ID NO: 5740). In some embodiments, the deaminase is a human APOBEC3G variant comprising a D316R_D317R mutation (SEQ ID NO: 5739). In some embodiments, the deaminase is a frantment of the human APOBEC3G and comprising mutations corresponding to the D316R_D317R mutations in SEQ ID NO: 275 (SEQ ID NO: 5741).

In some embodiments, the linker comprises a (GGGS)ₙ (SEQ ID NO: 265), (GGGGS)ₙ (SEQ ID NO: 5), a (G)ₙ, an (EAAAK)ₙ (SEQ ID NO: 6), a (GGS)ₙ, an SGSETPGTSESATPES (SEQ ID NO: 7), or an (XP)ₙ motif, or a combination of any of these, wherein n is independently an integer between 1 and 30.

Suitable UGI protein and nucleotide sequences are provided herein and additional suitable UGI sequences are known to those in the art, and include, for example, those published in Wang et al., Uracil-DNA glycosylase inhibitor gene of bacteriophage PBS2 encodes a binding protein specific for uracil-DNA glycosylase. J. Biol. Chem. 264:1163-1171(1989); Lundquist et al., Site-directed mutagenesis and characterization of uracil-DNA glycosylase inhibitor protein. Role of specific carboxylic amino acids in complex formation with Escherichia coli uracil-DNA glycosylase. J. Biol. Chem. 272:21408-21419(1997); Ravishankar et al., X-ray analysis of a complex of Escherichia coli uracil DNA glycosylase (EcUDG) with a proteinaceous inhibitor. The structure elucidation of a prokaryotic UDG. Nucleic Acids Res. 26:4880-4887(1998); and Putnam et al., Protein mimicry of DNA from crystal structures of the uracil-DNA glycosylase inhibitor protein and its complex with Escherichia coli uracil-DNA glycosylase. J. Mol. Biol. 287:331-346(1999), the entire contents of which are incorporated herein by reference. In some embodiments, the optional linker comprises a (GGS)ₙ motif, wherein n is 1, 2, 3, 4, 5, 6, 7, 8, 9, 19, 11, 12, 13, 14, 15, 16, 17, 18, 19, or 20. In some embodiments, the optional linker comprises a (GGS)n motif, wherein n is 1, 3, or 7. In some embodiments, the optional linker comprises the amino acid sequence SGSETPGTSESATPES (SEQ ID NO: 7), which is also referred to as the XTEN linker in the Examples.

In some embodiments, a Cas9 nickase may further facilitate the removal of a base on the non-edited strand in an organism whose genome is edited in vivo. The Cas9 nickase, as described herein, may comprise a D10A mutation in SEQ ID NO: 10, or a corresponding mutation in any of SEQ ID NOs: 11-260. In some embodiments, the Cas9 nickase of this disclosure may comprise a histidine at mutation 840 of SEQ ID NO: 10, or a corresponding residue in any of SEQ ID NOs: 11-260. Such fusion proteins comprising the Cas9 nickase, can cleave a single strand of the target DNA sequence, e.g., the strand that is not being edited. Without wishing to be bound by any particular theory, this cleavage may inhibit mis-match repair mechanisms that reverse a C to U edit made by the deaminase.

Cas9 Complexes with Guide RNAs

Some aspects of this disclosure provide complexes comprising any of the fusion proteins provided herein, and a guide RNA bound to a Cas9 domain (e.g., a dCas9, a nuclease active Cas9, or a Cas9 nickase) of fusion protein.

In some embodiments, the guide RNA is from 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence. In some embodiments, the target sequence is a DNA sequence. In some embodiments, the target sequence is a sequence in the genome of a mammal. In some embodiments, the target sequence is a sequence in the genome of a human. In some embodiments, the 3′ end of the target sequence is immediately adjacent to a canonical PAM sequence (NGG). In some embodiments, the guide RNA is complementary to a sequence associated with a disease or disorder. In some embodiments, the guide RNA is complementary to a sequence associated with a disease or disorder having a mutation in a gene selected from the genes disclosed in any one of Tables 1-3. In some embodiments, the guide RNA comprises a nucleotide sequence of any one of the guide sequences provided in Table 2 or Table 3. Exemplary sequences in the human genome that may be targeted by the complexes of this disclosure are provided herein in Tables 1-3.

### Methods of Using Cas9 Fusion Proteins

Some aspects of this disclosure provide methods of using the Cas9 proteins, fusion proteins, or complexes provided herein. For example, some aspects of this disclosure provide methods comprising contacting a DNA molecule (a) with any of the Cas9 proteins or fusion proteins provided herein, and with at least one guide RNA, wherein the guide RNA is about 15-100 nucleotides long and comprises a sequence of at least 10 contiguous nucleotides that is complementary to a target sequence; or (b) with a Cas9 protein, a Cas9 fusion protein, or a Cas9 protein or fusion protein complex with at least one gRNA as provided herein. In some embodiments, the 3′ end of the target sequence is not immediately adjacent to a canonical PAM sequence (NGG). In some embodiments, the 3′ end of the target sequence is immediately adjacent to an AGC, GAG, TTT, GTG, or CAA sequence.

In some embodiments, the target DNA sequence comprises a sequence associated with a disease or disorder. In some embodiments, the target DNA sequence comprises a point mutation associated with a disease or disorder. In some embodiments, the activity of the Cas9 protein, the Cas9 fusion protein, or the complex results in a correction of the point mutation. In some embodiments, the target DNA sequence comprises a T→C point mutation associated with a disease or disorder, and wherein the deamination of the mutant C base results in a sequence that is not associated with a disease or disorder. In some embodiments, the target DNA sequence encodes a protein and wherein the point mutation is in a codon and results in a change in the amino acid encoded by the mutant codon as compared to the wild-type codon. In some embodiments, the deamination of the mutant C results in a change of the amino acid encoded by the mutant codon. In some embodiments, the deamination of the mutant C results in the codon encoding the wild-type amino acid. In some embodiments, the contacting is in vivo in a subject. In some embodiments, the subject has or has been diagnosed with a disease or disorder. In some embodiments, the disease or disorder is cystic fibrosis, phenylketonuria, epidermolytic hyperkeratosis (EHK), Charcot-Marie-Toot disease type 4J, neuroblastoma (NB), von Willebrand disease (vWD), myotonia congenital, hereditary renal amyloidosis, dilated cardiomyopathy (DCM), hereditary lymphedema, familial Alzheimer's disease, HIV, Prion disease, chronic infantile neurologic cutaneous articular syndrome (CINCA), desmin-related myopathy (DRM), a neoplastic disease associated with a mutant PI3KCA protein, a mutant CTNNB1 protein, a mutant HRAS protein, or a mutant p53 protein.

Some embodiments provide methods for using the Cas9 DNA editing fusion proteins provided herein. In some embodiments, the fusion protein is used to introduce a point mutation into a nucleic acid by deaminating a target nucleobase, e.g., a C residue. In some embodiments, the deamination of the target nucleobase results in the correction of a genetic defect, e.g., in the correction of a point mutation that leads to a loss of function in a gene product. In some embodiments, the genetic defect is associated with a disease or disorder, e.g., a lysosomal storage disorder or a metabolic disease, such as, for example, type I diabetes. In some embodiments, the methods provided herein are used to introduce a deactivating point mutation into a gene or allele that encodes a gene product that is associated with a disease or disorder. For example, in some embodiments, methods are provided herein that employ a Cas9 DNA editing fusion protein to introduce a deactivating point mutation into an oncogene (e.g., in the treatment of a proliferative disease). A deactivating mutation may, in some embodiments, generate a premature stop codon in a coding sequence, which results in the expression of a truncated gene product, e.g., a truncated protein lacking the function of the full-length protein.

In some embodiments, the purpose of the methods provide herein is to restore the function of a dysfunctional gene via genome editing. The Cas9 deaminase fusion proteins provided herein can be validated for gene editing-based human therapeutics in vitro, e.g., by correcting a disease-associated mutation in human cell culture. It will be understood by the skilled artisan that the fusion proteins provided herein, e.g., the fusion proteins comprising a Cas9 domain and a nucleic acid deaminase domain can be used to correct any single point T→C or A→G mutation. In the first case, deamination of the mutant C back to U corrects the mutation, and in the latter case, deamination of the C that is base-paired with the mutant G, followed by a round of replication, corrects the mutation.

An exemplary disease-relevant mutation that can be corrected by the provided fusion proteins in vitro or in vivo is the H1047R (A3140G) polymorphism in the PI3KCA protein. The phosphoinositide-3-kinase, catalytic alpha subunit (PI3KCA) protein acts to phosphorylate the 3-OH group of the inositol ring of phosphatidylinositol. The PI3KCA gene has been found to be mutated in many different carcinomas, and thus it is considered to be a potent oncogene.³⁷ In fact, the A3140G mutation is present in several NCI-60 cancer cell lines, such as, for example, the HCT116, SKOV3, and T47D cell lines, which are readily available from the American Type Culture Collection (ATCC).³⁸

In some embodiments, a cell carrying a mutation to be corrected, e.g., a cell carrying a point mutation, e.g., an A3140G point mutation in exon 20 of the PI3KCA gene, resulting in a H1047R substitution in the PI3KCA protein, is contacted with an expression construct encoding a Cas9 deaminase fusion protein and an appropriately designed sgRNA targeting the fusion protein to the respective mutation site in the encoding PI3KCA gene. Control experiments can be performed where the sgRNAs are designed to target the fusion enzymes to non-C residues that are within the PI3KCA gene. Genomic DNA of the treated cells can be extracted, and the relevant sequence of the PI3KCA genes PCR amplified and sequenced to assess the activities of the fusion proteins in human cell culture.

It will be understood that the example of correcting point mutations in PI3KCA is provided for illustration purposes and is not meant to limit the instant disclosure. The skilled artisan will understand that the instantly disclosed DNA-editing fusion proteins can be used to correct other point mutations and mutations associated with other cancers and with diseases other than cancer including other proliferative diseases.

The successful correction of point mutations in disease-associated genes and alleles opens up new strategies for gene correction with applications in therapeutics and basic research. Site-specific single-base modification systems like the disclosed fusions of Cas9 and deaminase enzymes or domains also have applications in “reverse” gene therapy, where certain gene functions are purposely suppressed or abolished. In these cases, site-specifically mutating Trp (TGG), Gln (CAA and CAG), or Arg (CGA) residues to premature stop codons (TAA, TAG, TGA) can be used to abolish protein function in vitro, ex vivo, or in vivo.

The instant disclosure provides methods for the treatment of a subject diagnosed with a disease associated with or caused by a point mutation that can be corrected by a Cas9 DNA editing fusion protein provided herein. For example, in some embodiments, a method is provided that comprises administering to a subject having such a disease, e.g., a cancer associated with a PI3KCA point mutation as described above, an effective amount of a Cas9 deaminase fusion protein that corrects the point mutation or introduces a deactivating mutation into the disease-associated gene. In some embodiments, the disease is a proliferative disease. In some embodiments, the disease is a genetic disease. In some embodiments, the disease is a neoplastic disease. In some embodiments, the disease is a metabolic disease. In some embodiments, the disease is a lysosomal storage disease. Other diseases that can be treated by correcting a point mutation or introducing a deactivating mutation into a disease-associated gene will be known to those of skill in the art, and the disclosure is not limited in this respect.

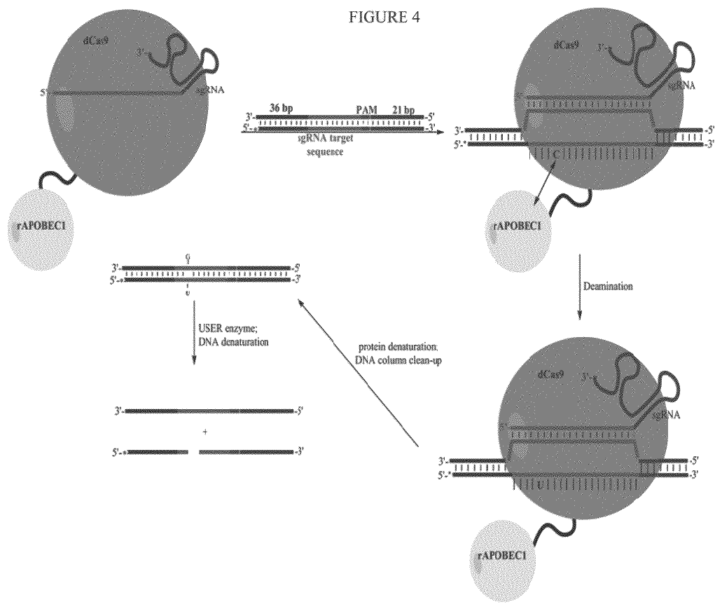

The instant disclosure provides methods for the treatment of additional diseases or disorders, e.g., diseases or disorders that are associated or caused by a point mutation that can be corrected by deaminase-mediated gene editing. Some such diseases are described herein, and additional suitable diseases that can be treated with the strategies and fusion proteins provided herein will be apparent to those of skill in the art based on the instant disclosure. Exemplary suitable diseases and disorders are listed below. It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues. Exemplary suitable diseases and disorders include, without limitation, cystic fibrosis (see, e.g., Schwank et al., Functional repair of CFTR by CRISPR/Cas9 in intestinal stem cell organoids of cystic fibrosis patients. Cell stem cell. 2013; 13: 653-658; and Wu et. al., Correction of a genetic disease in mouse via use of CRISPR-Cas9. Cell stem cell. 2013; 13: 659-662, neither of which uses a deaminase fusion protein to correct the genetic defect); phenylketonuria—e.g., phenylalanine to serine mutation at position 835 (mouse) or 240 (human) or a homologous residue in phenylalanine hydroxylase gene (T>C mutation)—see, e.g., McDonald et al., Genomics. 1997; 39:402-405; Bernard-Soulier syndrome (BSS)—e.g., phenylalanine to serine mutation at position 55 or a homologous residue, or cysteine to arginine at residue 24 or a homologous residue in the platelet membrane glycoprotein IX (T>C mutation)—see, e.g., Noris et al., British Journal of Haematology. 1997; 97: 312-320, and Ali et al., Hematol. 2014; 93: 381-384; epidermolytic hyperkeratosis (EHK)—e.g., leucine to proline mutation at position 160 or 161 (if counting the initiator methionine) or a homologous residue in keratin 1 (T>C mutation)—see, e.g., Chipev et al., Cell. 1992; 70: 821-828, see also accession number P04264 in the UNIPROT database at www[dot]uniprot[dot]org; chronic obstructive pulmonary disease (COPD)—e.g., leucine to proline mutation at position 54 or 55 (if counting the initiator methionine) or a homologous residue in the processed form of α₁-antitrypsin or residue 78 in the unprocessed form or a homologous residue (T>C mutation)—see, e.g., Poller et al., Genomics. 1993; 17: 740-743, see also accession number P01011 in the UNIPROT database; Charcot-Marie-Toot disease type 4J—e.g., isoleucine to threonine mutation at position 41 or a homologous residue in FIG. 4 (T>C mutation)—see, e.g., Lenk et al., PLoS Genetics. 2011; 7: e1002104; neuroblastoma (NB)—e.g., leucine to proline mutation at position 197 or a homologous residue in Caspase-9 (T>C mutation)—see, e.g., Kundu et al., 3 Biotech. 2013, 3:225-234; von Willebrand disease (vWD)—e.g., cysteine to arginine mutation at position 509 or a homologous residue in the processed form of von Willebrand factor, or at position 1272 or a homologous residue in the unprocessed form of von Willebrand factor (T>C mutation)—see, e.g., Lavergne et al., Br. J. Haematol. 1992, see also accession number P04275 in the UNIPROT database; 82: 66-72; myotonia congenital—e.g., cysteine to arginine mutation at position 277 or a homologous residue in the muscle chloride channel gene CLCN1 (T>C mutation)—see, e.g., Weinberger et al., The J. of Physiology. 2012; 590: 3449-3464; hereditary renal amyloidosis—e.g., stop codon to arginine mutation at position 78 or a homologous residue in the processed form of apolipoprotein AII or at position 101 or a homologous residue in the unprocessed form (T>C mutation)—see, e.g., Yazaki et al., Kidney Int. 2003; 64: 11-16; dilated cardiomyopathy (DCM)—e.g., tryptophan to Arginine mutation at position 148 or a homologous residue in the FOXD4 gene (T>C mutation), see, e.g., Minoretti et. al., Int. J. of Mol. Med. 2007; 19: 369-372; hereditary lymphedema—e.g., histidine to arginine mutation at position 1035 or a homologous residue in VEGFR3 tyrosine kinase (A>G mutation), see, e.g., Irrthum et al., Am. J. Hum. Genet. 2000; 67: 295-301; familial Alzheimer's disease—e.g., isoleucine to valine mutation at position 143 or a homologous residue in presenilin1 (A>G mutation), see, e.g., Gallo et. al., J. Alzheimer's disease. 2011; 25: 425-431; Prion disease—e.g., methionine to valine mutation at position 129 or a homologous residue in prion protein (A>G mutation)—see, e.g., Lewis et. al., J. of General Virology. 2006; 87: 2443-2449; chronic infantile neurologic cutaneous articular syndrome (CINCA)—e.g., Tyrosine to Cysteine mutation at position 570 or a homologous residue in cryopyrin (A>G mutation)—see, e.g., Fujisawa et. al. Blood. 2007; 109: 2903-2911; and desmin-related myopathy (DRM)—e.g., arginine to glycine mutation at position 120 or a homologous residue in αβ crystallin (A>G mutation)—see, e.g., Kumar et al., J. Biol. Chem. 1999; 274: 24137-24141. The entire contents of all references and database entries is incorporated herein by reference.

The instant disclosure provides lists of genes comprising pathogenic T>C or A>G mutations. Provided herein, are the names of these genes, their respective SEQ ID NOs, their gene IDs, and sequences flanking the mutation site. (Tables 2 and 3). In some instances, the gRNA sequences that can be used to correct the mutations in these genes are disclosed (Tables 2 and 3).

In some embodiments, a Cas9-deaminase fusion protein recognizes canonical PAMs and therefore can correct the pathogenic T>C or A>G mutations with canonical PAMs, e.g., NGG (listed in Tables 2 and 3, SEQ ID NOs: 2540-2702 and 5084-5260), respectively, in the flanking sequences. For example, the Cas9 proteins that recognize canonical PAMs comprise an amino acid sequence that is at least 90% identical to the amino acid sequence of Streptococcus pyogenes Cas9 as provided by SEQ ID NO: 10, or to a fragment thereof comprising the RuvC and HNH domains of SEQ ID NO: 10.

It will be apparent to those of skill in the art that in order to target a Cas9:nucleic acid editing enzyme/domain fusion protein as disclosed herein to a target site, e.g., a site comprising a point mutation to be edited, it is typically necessary to co-express the Cas9:nucleic acid editing enzyme/domain fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein. In some embodiments, the guide RNA comprises a structure 5′-[guide sequence]-guuuuagagcuagaaauagcaaguuaaaauaaaggcuaguccguuaucaacuugaaaaaguggcaccgagucggugcuuuuu-3′ (SEQ ID NO: 601), wherein the guide sequence comprises a sequence that is complementary to the target sequence. The guide sequence is typically 20 nucleotides long. The sequences of suitable guide RNAs for targeting Cas9:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting Cas9:nucleic acid editing enzyme/domain fusion proteins to specific target sequences are provided below.

### Base Editor Efficiency

Some aspects of the disclosure are based on the recognition that any of the base editors provided herein are capable of modifying a specific nucleotide base without generating a significant proportion of indels. An “indel”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g. mutate or deaminate) a specific nucleotide within a nucleic acid, without generating a large number of insertions or deletions (i.e., indels) in the nucleic acid. In certain embodiments, any of the base editors provided herein are capable of generating a greater proportion of intended modifications (e.g., point mutations or deaminations) versus indels. In some embodiments, the base editors provided herein are capable of generating a ratio of intended point mutations to indels that is greater than 1:1. In some embodiments, the base editors provided herein are capable of generating a ratio of intended point mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method, for example the methods used in the below Examples.

In some embodiments, the base editors provided herein are capable of limiting formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein are capable of limiting the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. The number of indels formed at a nucleic acid region may depend on the amount of time a nucleic acid (e.g., a nucleic acid within the genome of a cell) is exposed to a base editor. In some embodiments, an number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base editor.

Some aspects of the disclosure are based on the recognition that any of the base editors provided herein are capable of efficiently generating an intended mutation, such as a point mutation, in a nucleic acid (e.g. a nucleic acid within a genome of a subject) without generating a significant number of unintended mutations, such as unintended point mutations. In some embodiments, a intended mutation is a mutation that is generated by a specific base editor bound to a gRNA, specifically designed to generate the intended mutation. In some embodiments, the intended mutation is a mutation associated with a disease or disorder. In some embodiments, the intended mutation is a cytosine (C) to thymine (T) point mutation associated with a disease or disorder. In some embodiments, the intended mutation is a guanine (G) to adenine (A) point mutation associated with a disease or disorder. In some embodiments, the intended mutation is a cytosine (C) to thymine (T) point mutation within the coding region of a gene. In some embodiments, the intended mutation is a guanine (G) to adenine (A) point mutation within the coding region of a gene. In some embodiments, the intended mutation is a point mutation that generates a stop codon, for example, a premature stop codon within the coding region of a gene. In some embodiments, the intended mutation is a mutation that eliminates a stop codon. In some embodiments, the intended mutation is a mutation that alters the splicing of a gene. In some embodiments, the intended mutation is a mutation that alters the regulatory sequence of a gene (e.g., a gene promotor or gene repressor). In some embodiments, any of the base editors provided herein are capable of generating a ratio of intended mutations to unintended mutations (e.g., intended point mutations:unintended point mutations) that is greater than 1:1. In some embodiments, any of the base editors provided herein are capable of generating a ratio of intended mutations to unintended mutations (e.g., intended point mutations:unintended point mutations) that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more. It should be appreciated that the characteristics of the base editors described in the “Base Editor Efficiency” section, herein, may be applied to any of the fusion proteins, or methods of using the fusion proteins provided herein.

### Methods for Editing Nucleic Acids

Some aspects of the disclosure provide methods for editing a nucleic acid. In some embodiments, the method is a method for editing a nucleobase of a nucleic acid (e.g., a base pair of a double-stranded DNA sequence). In some embodiments, the method comprises the steps of: a) contacting a target region of a nucleic acid (e.g., a double-stranded DNA sequence) with a complex comprising a base editor (e.g., a Cas9 domain fused to a cytidine deaminase domain) and a guide nucleic acid (e.g., gRNA), wherein the target region comprises a targeted nucleobase pair, b) inducing strand separation of said target region, c) converting a first nucleobase of said target nucleobase pair in a single strand of the target region to a second nucleobase, and d) cutting no more than one strand of said target region, where a third nucleobase complementary to the first nucleobase base is replaced by a fourth nucleobase complementary to the second nucleobase; and the method results in less than 20% indel formation in the nucleic acid. It should be appreciated that in some embodiments, step b is omitted. In some embodiments, the first nucleobase is a cytosine. In some embodiments, the second nucleobase is a deaminated cytosine, or a uracil. In some embodiments, the third nucleobase is a guanine. In some embodiments, the fourth nucleobase is an adenine. In some embodiments, the first nucleobase is a cytosine, the second nucleobase is a deaminated cytosine, or a uracil, the third nucleobase is a guanine, and the fourth nucleobase is an adenine. In some embodiments, the method results in less than 19%, 18%, 16%, 14%, 12%, 10%, 8%, 6%, 4%, 2%, 1%, 0.5%, 0.2%, or less than 0.1% indel formation. In some embodiments, the method further comprises replacing the second nucleobase with a fifth nucleobase that is complementary to the fourth nucleobase, thereby generating an intended edited base pair (e.g., C:G→T:A). In some embodiments, the fifth nucleobase is a thymine. In some embodiments, at least 5% of the intended basepaires are edited. In some embodiments, at least 10%, 15%, 20%, 25%, 30%, 35%, 40%, 45%, or 50% of the intended basepaires are edited.

In some embodiments, the ratio of intended products to unintended products in the target nucleotide is at least 2:1, 5:1, 10:1, 20:1, 30:1, 40:1, 50:1, 60:1, 70:1, 80:1, 90:1, 100:1, or 200:1, or more. In some embodiments, the ratio of intended point mutation to indel formation is greater than 1:1, 10:1, 50:1, 100:1, 500:1, or 1000:1, or more. In some embodiments, the cut single strand (nicked strand) is hybridized to the guide nucleic acid. In some embodiments, the cut single strand is opposite to the strand comprising the first nucleobase. In some embodiments, the base editor comprises a Cas9 domain. In some embodiments, the first base is cytosine, and the second base is not a G, C, A, or T. In some embodiments, the second base is uracil. In some embodiments, the first base is cytosine. In some embodiments, the second base is not a G, C, A, or T. In some embodiments, the second base is uracil. In some embodiments, the base editor inhibits base escision repair of the edited strand. In some embodiments, the base editor protects or binds the non-edited strand. In some embodiments, the base editor comprises UGI activity. In some embodiments, the base editor comprises nickase activity. In some embodiments, the intended edited basepair is upstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited basepair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site. In some embodiments, the nucleobase editor comprises a linker. In some embodiments, the linker is 1-25 amino acids in length. In some embodiments, the linker is 5-20 amino acids in length. In some embodiments, linker is 10, 11, 12, 13, 14, 15, 16, 17, 18, 19, or 20 amino acids in length. In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair is within the target window. In some embodiments, the target window comprises the intended edited base pair. In some embodiments, the method is performed using any of the base editors provided herein. In some embodiments, a target window is a deamination window

In some embodiments, the disclosure provides methods for editing a nucleotide. In some embodiments, the disclosure provides a method for editing a nucleobase pair of a double-stranded DNA sequence. In some embodiments, the method comprises a) contacting a target region of the double-stranded DNA sequence with a complex comprising a base editor and a guide nucleic acid (e.g., gRNA), where the target region comprises a target nucleobase pair, b) inducing strand separation of said target region, c) converting a first nucleobase of said target nucleobase pair in a single strand of the target region to a second nucleobase, d) cutting no more than one strand of said target region, wherein a third nucleobase complementary to the first nucleobase base is replaced by a fourth nucleobase complementary to the second nucleobase, and the second nucleobase is replaced with a fifth nucleobase that is complementary to the fourth nucleobase, thereby generating an intended edited basepair, wherein the efficiency of generating the intended edited basepair is at least 5%. It should be appreciated that in some embodiments, step b is omitted. In some embodiments, at least 5% of the intended basepaires are edited. In some embodiments, at least 10%, 15%, 20%, 25%, 30%, 35%, 40%, 45%, or 50% of the intended basepaires are edited. In some embodiments, the method causes less than 19%, 18%, 16%, 14%, 12%, 10%, 8%, 6%, 4%, 2%, 1%, 0.5%, 0.2%, or less than 0.1% indel formation. In some embodiments, the ratio of intended product to unintended products at the target nucleotide is at least 2:1, 5:1, 10:1, 20:1, 30:1, 40:1, 50:1, 60:1, 70:1, 80:1, 90:1, 100:1, or 200:1, or more. In some embodiments, the ratio of intended point mutation to indel formation is greater than 1:1, 10:1, 50:1, 100:1, 500:1, or 1000:1, or more. In some embodiments, the cut single strand is hybridized to the guide nucleic acid. In some embodiments, the cut single strand is opposite to the strand comprising the first nucleobase. In some embodiments, the first base is cytosine. In some embodiments, the second nucleobase is not G, C, A, or T. In some embodiments, the second base is uracil. In some embodiments, the base editor inhibits base escision repair of the edited strand. In some embodiments, the base editor protects or binds the non-edited strand. In some embodiments, the nucleobase editor comprises UGI activity. In some embodiments, the nucleobase edit comprises nickase activity. In some embodiments, the intended edited basepair is upstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited basepair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site. In some embodiments, the nucleobase editor comprises a linker. In some embodiments, the linker is 1-25 amino acids in length. In some embodiments, the linker is 5-20 amino acids in length. In some embodiments, the linker is 10, 11, 12, 13, 14, 15, 16, 17, 18, 19, or 20 amino acids in length. In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair occurs within the target window. In some embodiments, the target window comprises the intended edited base pair. In some embodiments, the nucleobase editor is any one of the base editors provided herein.

### Kits, Vectors, Cells

Some aspects of this disclosure provide kits comprising a nucleic acid construct, comprising (a) a nucleotide sequence encoding a Cas9 protein or a Cas9 fusion protein as provided herein; and (b) a heterologous promoter that drives expression of the sequence of (a). In some embodiments, the kit further comprises an expression construct encoding a guide RNA backbone, wherein the construct comprises a cloning site positioned to allow the cloning of a nucleic acid sequence identical or complementary to a target sequence into the guide RNA backbone.

Some aspects of this disclosure provide polynucleotides encoding a Cas9 protein of a fusion protein as provided herein. Some aspects of this disclosure provide vectors comprising such polynucleotides. In some embodiments, the vector comprises a heterologous promoter driving expression of polynucleotide.

Some aspects of this disclosure provide cells comprising a Cas9 protein, a fusion protein, a nucleic acid molecule encoding the fusion protein, a complex comprise the Cas9 protein and the gRNA, and/or a vector as provided herein.

The description of exemplary embodiments of the reporter systems above is provided for illustration purposes only and not meant to be limiting. Additional reporter systems, e.g., variations of the exemplary systems described in detail above, are also embraced by this disclosure.

### EXAMPLES

### Example 1: Cas9 Deaminase Fusion Proteins

A number of Cas9:Deaminase fusion proteins were generated and deaminase activity of the generated fusions was characterized. The following deaminases were tested:

Human AID (hAID):
(SEQ ID NO: 607)
MDSLLMNRRKFLYQFKNVRWAKGRRETYLCYVVKRRDSATSFSLD
FGYLRNKNGCHVELLFLRYISDWDLDPGRCYRVTWFTSWSPCYDC
ARHVADFLRGNPYLSLRIFTARLYFCEDRKAEPEGLRRLHRAGVQ
IAIMTFKDYFYCWNTFVENHERTFKAWEGLHENSVRLSRQLRRIL
LPLYEVDDLRDAFRTLGLLD
Human AID-DC (hAID-DC, truncated version
of hAID with 7-fold increased activity):
(SEQ ID NO: 608)
MDSLLMNRRKFLYQFKNVRWAKGRRETYLCYVVKRRDSATSFSLD
FGYLRNKNGCHVELLFLRYISDWDLDPGRCYRVTWFTSWSPCYDC
ARHVADFLRGNPNLSLRIFTARLYFCEDRKAEPEGLRRLHRAGVQ
IAIMTFKDYFYCWNTFVENHERTFKAWEGLHENSVRLSRQLRRIL
L
Rat APOBEC1 (rAPOBEC1):
(SEQ ID NO: 284)
MSSETGPVAVDPTLRRRIEPHEFEVFFDPRELRKETCLLYEINWG
GRHSIWRHTSQNTNKHVEVNFIEKFTTERYFCPNTRCSITWFLSW
SPCGECSRAITEFLSRYPHVTLFIYIARLYHHADPRNRQGLRDLI
SSGVTIQIMTEQESGYCWRNFVNYSPSNEAHWPRYPHLWVRLYVL
ELYCIILGLPPCLNILRRKQPQLTFFTIALQSCHYQRLPPHILWA
TGLK
Human APOBEC1 (hAPOBEC1)
(SEQ ID NO: 5724)
MTSEKGPSTGDPTLRRRIEPWEFDVFYDPRELRKEACLLYEIKWG
MSRKIWRSSGKNTTNHVEVNFIKKFTSERDFHPSMSCSITWFLSW
SPCWECSQAIREFLSRHPGVTLVIYVARLFWHMDQQNRQGLRDLV
NSGVTIQIMRASEYYHCWRNFVNYPPGDEAHWPQYPPLWMMLYAL
ELHCIILSLPPCLKISRRWQNHLTFFRLHLQNCHYQTIPPHILLA
TGLIHPSVAWR
Petromyzon marinus (Lamprey) CDA1 (pmCDA1):
(SEQ ID NO: 609)
MTDAEYVRIHEKLDIYTFKKQFFNNKKSVSHRCYVLFELKRRGER
RACFWGYAVNKPQSGTERGIHAEIFSIRKVEEYLRDNPGQFTINW
YSSWSPCADCAEKILEWYNQELRGNGHTLKIWACKLYYEKNARNQ
IGLWNLRDNGVGLNVMVSEHYQCCRKIFIQSSHNQLNENRWLEKT
LKRAEKRRSELSIMIQVKILHTTKSPAV
Human APOBEC3G (hAPOBEC3G):
(SEQ ID NO: 610)
MELKYHPEMRFFHWFSKWRKLHRDQEYEVTWYISWSPCTKCTRDM
ATFLAEDPKVTLTIFVARLYYFWDPDYQEALRSLCQKRDGPRATM
KIMNYDEFQHCWSKFVYSQRELFEPWNNLPKYYILLHIMLGEILR
HSMDPPTFTFNFNNEPWVRGRHETYLCYEVERMHNDTWVLLNQRR
GFLCNQAPHKHGFLEGRHAELCFLDVIPFWKLDLDQDYRVTCFTS
WSPCFSCAQEMAKFISKNKHVSLCIFTARIYDDQGRCQEGLRTLA
EAGAKISIMTYSEFKHCWDTFVDHQGCPFQPWDGLDEHSQDLSGR
LRAILQNQEN

Deaminase Activity on ssDNA. A USER (Uracil-Specific Excision Reagent) Enzyme-based assay for deamination was employed to test the activity of various deaminases on single-stranded DNA (ssDNA) substrates. USER Enzyme was obtained from New England Biolabs. An ssDNA substrate was provided with a target cytosine residue at different positions. Deamination of the ssDNA cytosine target residue results in conversion of the target cytosine to a uracil. The USER Enzyme excises the uracil base and cleaves the ssDNA backbone at that position, cutting the ssDNA substrate into two shorter fragments of DNA. In some assays, the ssDNA substrate is labeled on one end with a dye, e.g., with a 5′ Cy3 label (the * in the scheme below). Upon deamination, excision, and cleavage of the strand, the substrate can be subjected to electrophoresis, and the substrate and any fragment released from it can be visualized by detecting the label. Where Cy5 is images, only the fragment with the label will be visible via imaging.

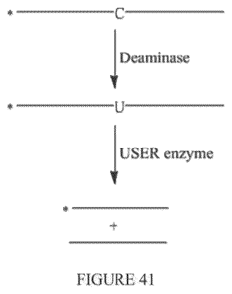

In one USER Enzyme assay, ssDNA substrates were used that matched the target sequences of the various deaminases tested. Expression cassettes encoding the deaminases tested were inserted into a CMV backbone plasmid that has been used previously in the lab (Addgene plasmid 52970). The deaminase proteins were expressed using a TNT Quick Coupled Transcription/Translation System (Promega) according to the manufacturers recommendations. After 90 min of incubation, 5 mL of lysate was incubated with 5′ Cy3-labeled ssDNA substrate and 1 unit of USER Enzyme (NEB) for 3 hours. The DNA was resolved on a 10% TBE PAGE gel and the DNA was imaged using Cy-dye imaging. A schematic representation of the USER Enzyme assay is shown in FIG. 41.

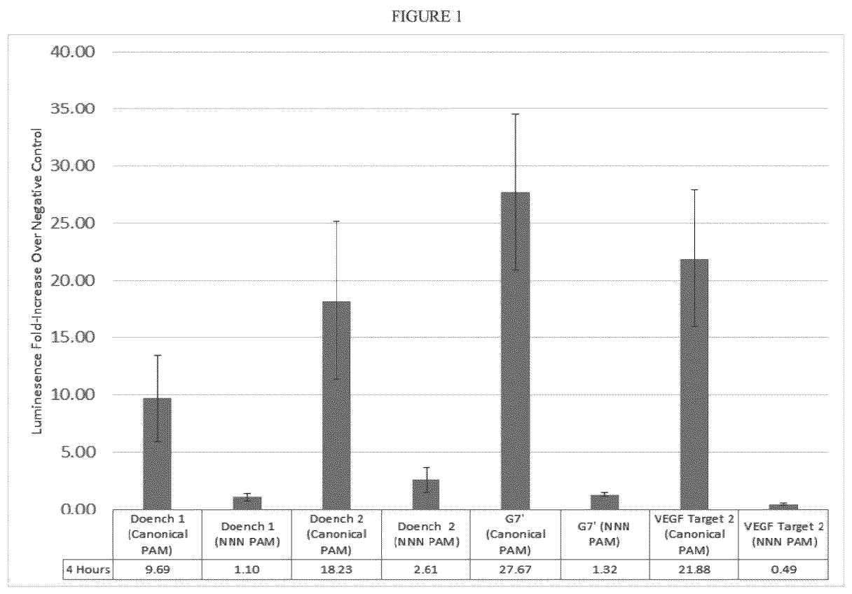

FIG. 1 shows the deaminase activity of the tested deaminases on ssDNA substrates, such as Doench 1, Doench 2, G7′ and VEGF Target 2. The rAPOBEC1 enzyme exhibited a substantial amount of deamination on the single-stranded DNA substrate with a canonical NGG PAM, but not with a negative control non-canonical NNN PAM.

Cas9 fusion proteins with APOBEC family deaminases were generated. The following fusion architectures were constructed and tested on ssDNA:

rAPOBEC1-GGS-dCas9 primary sequence
(SEQ ID NO: 611)
MSSETGPVAVDPTLRRRIEPHEFEVFFDPRELRKETCLLYEINWGGRHSIWRHTSQNTNK
HVEVNFIEKFTTERYFCPNTRCSITWFLSWSPCGECSRAITEFLSRYPHVTLFIYIARLYHH
ADPRNRQGLRDLISSGVTIQIMTEQESGYCWRNFVNYSPSNEAHWPRYPHLWVRLYVLE

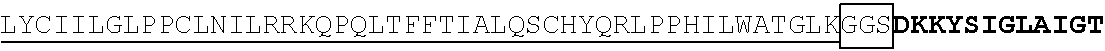

NSVGWAVITDEYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIVDEVAYHEKYP
TIYHLRKKLVDSTDKADLRLIYLALAHMIKFRGHFLIEGDLNPDNSDVDKLFIQLVQTYN
QLFEENPINASGVDAKAILSARLSKSRRLENLIAQLPGEKKNGLFGNLIALSLGLTPNFKS
NFDLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKD
NREKIEKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLFKT
NRKVTVKOLKEDYFKKIECFDSVEISGVEDRFNASLGTYHDLLKIIKDKDFLDNEENEDIL
EDIVLTLTLFEDREMIEERLKTYAHLFDDKVMKQLKRRRYTGWGRLSRKLINGIRDKQSG
KTILDFLKSDGFANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIEEGIKELGSQIL
KEHPVENTQLQNEKLYLYYLQNGRDMYVDQELDINRLSDYDVDAIVPQSFLKDDSIDNK
VLTRSDKNRGKSDNVPSEEVVKKMKNYWRQLLNAKLITQRKFDNLTKAERGGLSELDKA
GFIKRQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMPQVNIV
KKTEVQTGGFSKESILPKRNSDKLIARKKDWDPKKYGGFDSPTVAYSVLVVAKVEKGKSK
KLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEF
SKRVILADANLDKVLSAYNKHRDKPIREQAENIIHLFTLTNLGAPAAFKYFDTTIDRKRYT
STKEVLDATLIHQSITGLYETRIDLSQLGGD
rAPOBEC1-(GGS)3-dCas9 primary sequence
(SEQ ID NO: 612)
MSSETGPVAVDPTLRRRIEPHEFEVFFDPRELRKETCLLYEINWGGRHSIWRHTSONTNK
HVEVNFIEKFTTERYFCPNTRCSITWFLSWSPCGECSRAITEFLSRYPHVTLFIYIARLYHH
ADPRNROGLRDLISSGVTIQIMTEQESGYCWRNFVNYSPSNEAHWPRYPHLWVRLYVLE

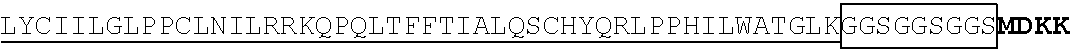

YSIGLAIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDE
VAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLG
LTPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGGA
SQEEFYKFIKPILEKMDGTEELLVKLNREDLLRKORTFDNGSIPHQIHLGELHAILRRQE
DFYPFLKDNREKIEKILTFRIPYYVGPLARGNSRFAWMTRKSEETITPWNFEEVVDKGAS
AQSFIERMTNFDKNLPNEKVLPKHSLLYEYFTVYNELTKVKYVTEGMRKPAFLSGEQKK
AIVDLLFKTNRKVTVKOLKEDYFKKIECFDSVEISGVEDRFNASLGTYHDLLKIIKDKDFL
DNEENEDILEDIVLTLTLFEDREMIEERLKTYAHLFDDKVMKOLKRRRYTGWGRLSRKLI
NGIRDKQSGKTILDFLKSDGFANRNFMQLIHDDSLTFKEDIQKAQVSGQGDSLHEHIANL
AGSPAIKKGILQTVKVVDELVKVMGRHKPENIVIEMARENQTTQKGQKNSRERMKRIEE
GIKELGSQILKEHPVENTQLQNEKLYLYYLQNGRDMYVDQELDINRLSDYDVDAIVPQSF
LKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVLVVA
KVEKGKSKKLKSVKELLGITIMERSSFEKNPIDFLEAKGYKEVKKDLIIKLPKYSLFELENG
RKRMLASAGELQKGNELALPSKYVNFLYLASHYEKLKGSPEDNEQKQLFVEQHKHYLDE
IIEQISEFSKRVILADANLDKVLSAYNKHRDKPIREQAENIIHLFTLTNLGAPAAFKYFDTT
IDRKRYTSTKEVLDATLIHQSITGLYETRIDLSQLGGD

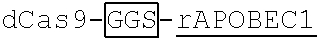

(SEQ ID NO: 613)
DKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVD
KLFIQLVQTYNQLFEENPINASGVDAKAILSARLSKSRRLENLIAQLPGEKKNGLFGNLIA
LSLGLTPNFKSNFDLAEDAKLQLSKDTYDDDLDNLLAQIGDQYADLFLAAKNLSDAILLS
DILRVNTEITKAPLSASMIKRYDEHHQDLTLLKALVRQQLPEKYKEIFFDQSKNGYAGYID
GGASQEEFYKFIKPILEKMDGTEELLVKLNREDLLRKQRTFDNGSIPHQIHLGELHAILR
RQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYHDLLKIIKDK
DFLDNEENEDILEDIVLTLTLFEDREMIEERLKTYAHLFDDKVMKOLKRRRYTGWGRLS
RKLINGIRDKQSGKTILDFLKSDGFANRNFMQLIHDDSLTFKEDIQKAQVSGQGDSLHEH
IANLAGSPAIKKGILQTVKVVDELVKVMGRHKPENIVIEMARENQTTQKGQKNSRERMK
RIEEGIKELGSQILKEHPVENTQLQNEKLYLYYLONGRDMYVDQELDINRLSDYDVDAIV
PQSFLKDDSIDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIREVKVITLKSKL
VSDFRKDFQFYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAYS
VLVVAKVEKGKSKKLKSVKELLGITIMERSSFEKNPIDFLEAKGYKEVKKDLIIKLPKYSLF
ELENGRKRMLASAGELQKGNELALPSKYVNFLYLASHYEKLKGSPEDNEQKQLFVEQHK
HYLDEIIEQISEFSKRVILADANLDKVLSAYNKHRDKPIREQAENIIHLFTLTNLGAPAAFK

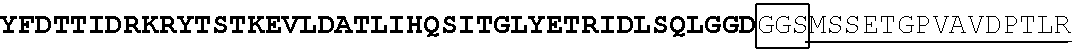

RRIEPHEFEVFFDPRELRKETCLLYEINWGGRHSIWRHTSONTNKHVEVNFIEKFTTERYF
CPNTRCSITWFLSWSPCGECSRAITEFLSRYPHVTLFIYIARLYHHADPRNROGLRDLISSG
VTIQIMTEQESGYCWRNFVNYSPSNEAHWPRYPHLWVRLYVLELYCIILGLPPCLNILRR
KQPQLTFFTIALQSCHYQRLPPHILWATGLK

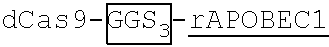

(SEQ ID NO: 614)
DKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVD
KLFIQLVQTYNQLFEENPINASGVDAKAILSARLSKSRRLENLIAQLPGEKKNGLFGNLIA
LSLGLTPNFKSNFDLAEDAKLQLSKDTYDDDLDNLLAQIGDQYADLFLAAKNLSDAILLS
DILRVNTEITKAPLSASMIKRYDEHHQDLTLLKALVRQQLPEKYKEIFFDQSKNGYAGYID
GGASQEEFYKFIKPILEKMDGTEELLVKLNREDLLRKQRTFDNGSIPHQIHLGELHAILR
RQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYHDLLKIIKDK
DFLDNEENEDILEDIVLTLTLFEDREMIEERLKTYAHLFDDKVMKQLKRRRYTGWGRLS
RKLINGIRDKQSGKTILDFLKSDGFANRNFMQLIHDDSLTFKEDIQKAQVSGQGDSLHEH
IANLAGSPAIKKGILQTVKVVDELVKVMGRHKPENIVIEMARENQTTQKGQKNSRERMK
RIEEGIKELGSQILKEHPVENTQLQNEKLYLYYLQNGRDMYVDQELDINRLSDYDVDAIV
PQSFLKDDSIDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIREVKVITLKSKL
VSDFRKDFQFYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAYS
VLVVAKVEKGKSKKLKSVKELLGITIMERSSFEKNPIDFLEAKGYKEVKKDLIIKLPKYSLF
ELENGRKRMLASAGELQKGNELALPSKYVNFLYLASHYEKLKGSPEDNEQKQLFVEQHK
HYLDEIIEQISEFSKRVILADANLDKVLSAYNKHRDKPIREQAENIIHLFTLTNLGAPAAFK

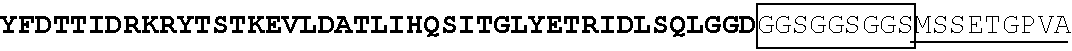

VDPTLRRRIEPHEFEVFFDPRELRKETCLLYEINWGGRHSIWRHTSONTNKHVEVNFIEKF
TTERYFCPNTRCSITWFLSWSPCGECSRAITEFLSRYPHVTLFIYIARLYHHADPRNRQGLR
DLISSGVTIQIMTEQESGYCWRNFVNYSPSNEAHWPRYPHLWVRLYVLELYCIILGLPPCL
NILRRKOPOLTFFTIALOSCHYQRLPPHILWATGLK

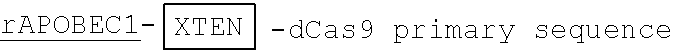

(SEQ ID NO: 615)
MSSETGPVAVDPTLRRRIEPHEFEVFFDPRELRKETCLLYEINWGGRHSIWRHTSONTNK
HVEVNFIEKFTTERYFCPNTRCSITWFLSWSPCGECSRAITEFLSRYPHVTLFIYIARLYHH
ADPRNROGLRDLISSGVTIQIMTEQESGYCWRNFVNYSPSNEAHWPRYPHLWVRLYVLE

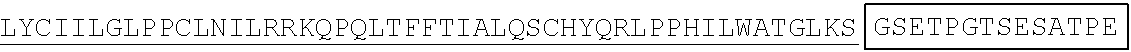

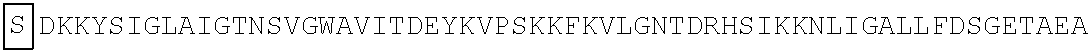

TRLKRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLAAKNLSDAILL
SDILRVNTEITKAPLSASMIKRYDEHHQDLTLLKALVRQQLPEKYKEIFFDQSKNGYAGYI
DGGASQEEFYKFIKPILEKMDGTEELLVKLNREDLLRKORTFDNGSIPHQIHLGELHAIL
RRQEDFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYHDLLKIIKDK
DFLDNEENEDILEDIVLTLTLFEDREMIEERLKTYAHLFDDKVMKOLKRRRYTGWGRLS
RKLINGIRDKQSGKTILDFLKSDGFANRNFMQLIHDDSLTFKEDIQKAQVSGQGDSLHEH
IANLAGSPAIKKGILQTVKVVDELVKVMGRHKPENIVIEMARENQTTQKGQKNSRERMK
RIEEGIKELGSQILKEHPVENTQLQNEKLYLYYLQNGRDMYVDQELDINRLSDYDVDAIV
PQSFLKDDSIDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIREVKVITLKSKL
VSDFRKDFQFYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAYS
VLVVAKVEKGKSKKLKSVKELLGITIMERSSFEKNPIDFLEAKGYKEVKKDLIIKLPKYSLF
ELENGRKRMLASAGELQKGNELALPSKYVNFLYLASHYEKLKGSPEDNEQKQLFVEQHK
HYLDEIIEQISEFSKRVILADANLDKVLSAYNKHRDKPIREQAENIIHLFTLTNLGAPAAFK
YFDTTIDRKRYTSTKEVLDATLIHQSITGLYETRIDLSQLGGD

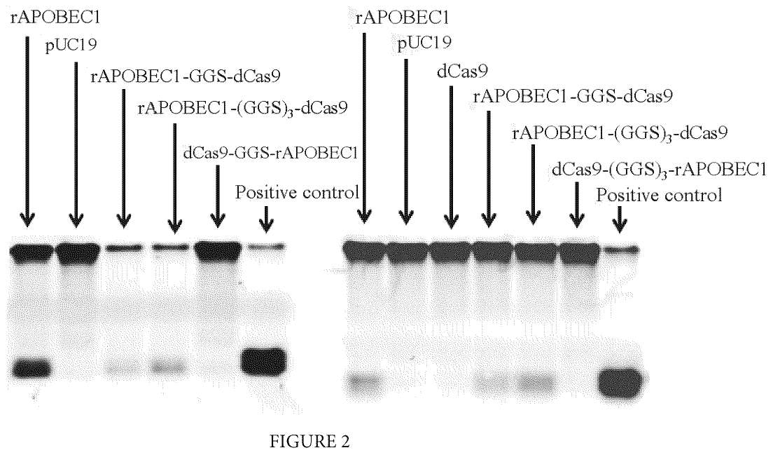

FIG. 2 shows that the N-terminal deaminase fusions showed significant activity on the single stranded DNA substrates. For this reason, only the N-terminal architecture was chosen for further experiments.

FIG. 3 illustrates double stranded DNA substrate binding by deaminase-dCas9:sgRNA complexes. A number of double stranded deaminase substrate sequences were generated. The sequences are provided below. The structures according to FIG. 3 are identified in these sequences (36bp: underlined, sgRNA target sequence: bold; PAM: boxed; 21bp: italicized). All substrates were labeled with a 5′-Cy3 label:

(SEQ ID NO: 616)

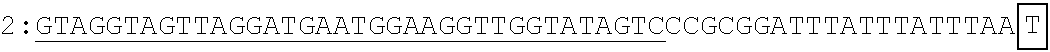

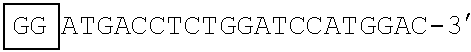

(SEQ ID NO: 617)

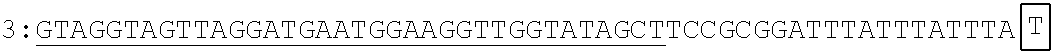

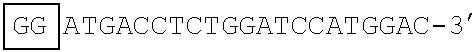

(SEQ ID NO: 618)

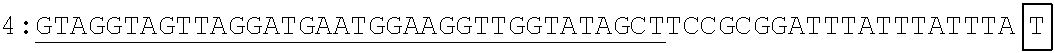

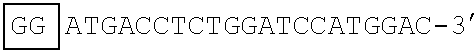

(SEQ ID NO: 619)

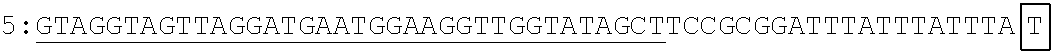

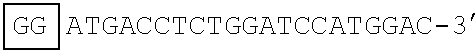

(SEQ ID NO: 620)

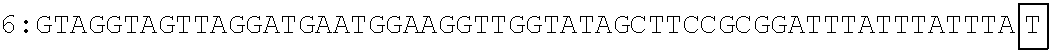

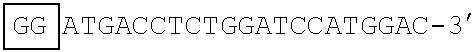

(SEQ ID NO: 621)

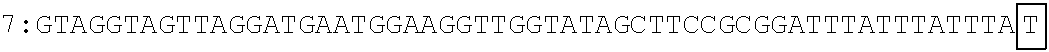

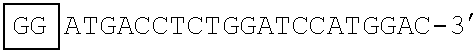

(SEQ ID NO: 622)

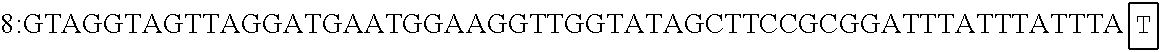

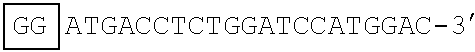

(SEQ ID NO: 623)

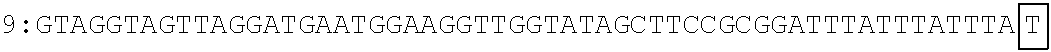

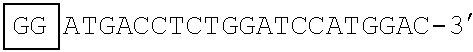

(SEQ ID NO: 624)
10:GTAGGTAGTTAGGATGAATGGAAGGTTGGTATAGCCATTATATTCCGCGGATTTAT

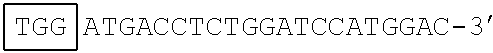

(SEQ ID NO: 625)
11:GTAGGTAGTTAGGATGAATGGAAGGTTGGTATAGCCTATTATATTCCGCGGATTTA

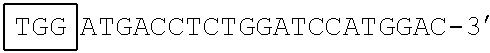

(SEQ ID NO: 626)
12:GTAGGTAGTTAGGATGAATGGAAGGTTGGTATAGCCTTATTATATTCCGCGGATTT

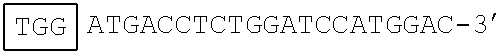

(SEQ ID NO: 627)
13:GTAGGTAGTTAGGATGAATGGAAGGTTGGTATAGCCATTATTATATTCCGCGGATT

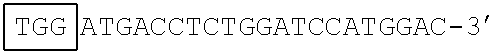

(SEQ ID NO: 628)
14:GTAGGTAGTTAGGATGAATGGAAGGTTGGTATAGCCTATTATTATATTCCGCGGAT

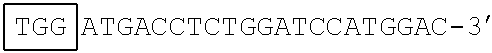

(SEQ ID NO: 629)
15: GTAGGTAGTTAGGATGAATGGAAGGTTGGTATAGCCATTATTATTATTACCGCGGA

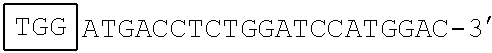

(SEQ ID NO: 630)

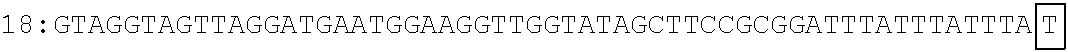

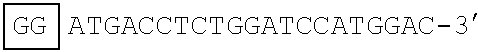

“-”:
(SEQ ID NO: 631)

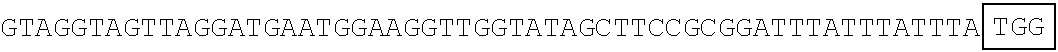

ATGACCTCTGGATCCATGGAC-3′
(SEQ ID NO: 632)

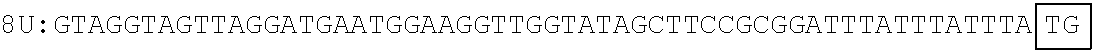

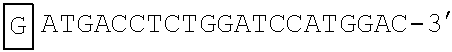

*In all substrates except for “8U”, the top strand in FIG. 3 is the
complement of the sequence specified here. In the case of “8U”, there is a
“G” opposite the U.

FIG. 4 shows the results of a double stranded DNA Deamination Assay. The fusions were expressed and purified with an N-terminal His6 tag via both Ni-NTA and sepharose chromatography. In order to assess deamination on dsDNA substrates, the various dsDNA substrates shown on the previous slide were incubated at a 1:8 dsDNA:fusion protein ratio and incubated at 37° C. for 2 hours. Once the dCas9 portion of the fusion binds to the DNA it blocks access of the USER enzyme to the DNA. Therefore, the fusion proteins were denatured following the incubation and the dsDNA was purified on a spin column, followed by incubation for 45 min with the USER Enzyme and resolution of the resulting DNA substrate and substrate fragments on a 10% TBE-urea gel.

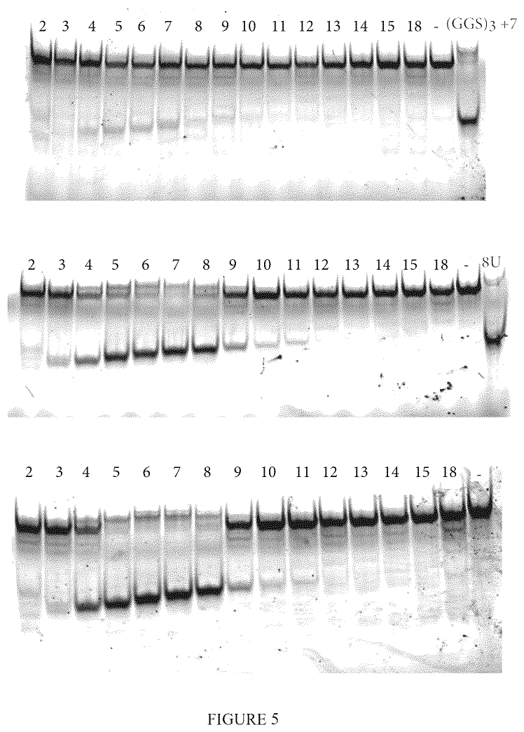

FIG. 5 demonstrates that Cas9 fusions can target positions 3-11 of double-stranded DNA target sequences (numbered according to the schematic in FIG. 3). Upper Gel: 1 μM rAPOBEC1-GGS-dCas9, 125 nM dsDNA, 1 eq sgRNA. Mid Gel: 1 μM rAPOBEC1-(GGS)₃-dCas9, 125 nM dsDNA, 1 eq sgRNA. Lower Gel: 1.85 μM rAPOBEC1-XTEN-dCas9, 125 nM dsDNA, 1 eq sgRNA. Based on the data from these gels, positions 3-11 (according to the numbering in FIG. 3) are sufficiently exposed to the activity of the deaminase to be targeted by the fusion proteins tested. Access of the deaminase to other positions is most likely blocked by the dCas9 protein.

The data further indicates that a linker of only 3 amino acids (GGS) is not optimal for allowing the deaminase to access the single stranded portion of the DNA. The 9 amino acid linker [(GGS)₃](SEQ ID NO: 596) and the more structured 16 amino acid linker (XTEN) allow for more efficient deamination.

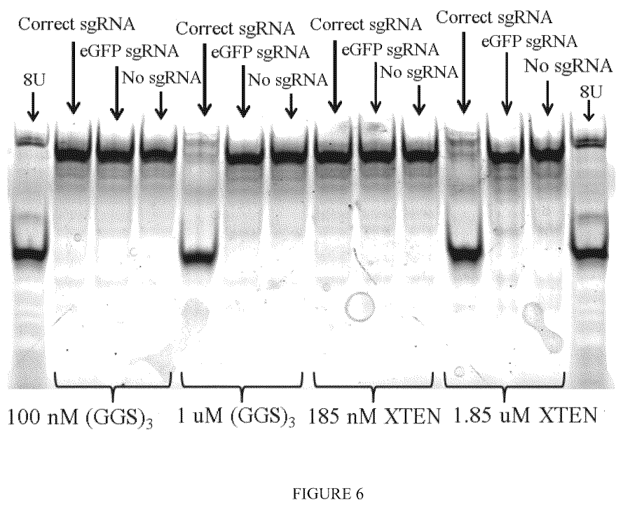

FIG. 6 demonstrates that the correct guide RNA, e.g., the correct sgRNA, is required for deaminase activity. The gel shows that fusing the deaminase to dCas9, the deaminase enzyme becomes sequence specific (e.g., using the fusion with an eGFP sgRNA results in no deamination), and also confers the capacity to the deaminase to deaminate dsDNA. The native substrate of the deaminase enzyme is ssDNA, and no deamination occurred when no sgRNA was added. This is consistent with reported knowledge that APOBEC deaminase by itself does not deaminate dsDNA. The data indicates that Cas9 opens the double-stranded DNA helix within a short window, exposing single-stranded DNA that is then accessible to the APOBEC deaminase for cytidine deamination. The sgRNA sequences used are provided below. sequences (36bp: underlined, sgRNA target sequence: bold; PAM: boxed; 21bp: italicized)

### DNA Sequence 8:

(SEQ ID NO: 633)
5′-Cy3-GTAGGTAGTTAGGATGAATGGAAGGTTGGTATAGCCATTATTCCGCGGATTTATT

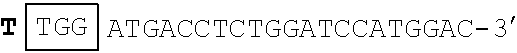

Correct sgRNA sequence (partial 3′ sequence):
(SEQ ID NO: 634)
5′-AUUAUUCCGCGGAUUUAUUUGUUUUAGAGCUAG ...-3′
eGFP sgRNA sequence (partial 3′-sequence):
(SEQ ID NO: 635)
5′-CGUAGGCCAGGGUGGUCACGGUUUUAGAGCUAG ...-3′

### Example 2: Deamination of DNA Target Sequence

Exemplary deamination targets. The dCas9:deaminase fusion proteins described herein can be delivered to a cell in vitro or ex vivo or to a subject in vivo and can be used to effect C to T or G to A transitions when the target nucleotide is in positions 3-11 with respect to a PAM. Exemplary deamination targets include, without limitation, the following: CCR5 truncations: any of the codons encoding Q93, Q102, Q186, R225, W86, or Q261 of CCR5 can be deaminated to generate a STOP codon, which results in a nonfunctional truncation of CCR5 with applications in HIV treatment. APOE4 mutations: mutant codons encoding C11R and C57R mutant APOE4 proteins can be deaminated to revert to the wild-type amino acid with applications in Alzheimer's treatment. eGFP truncations: any of the codons encoding Q158, Q184, Q185 can be deaminated to generate a STOP codon, or the codon encoding M1 can be deaminated to encode I, all of which result in loss of eGFP fluorescence, with applications in reporter systems. eGFP restoration: a mutant codon encoding T65A or Y66C mutant GFP, which does not exhibit substantial fluorescence, can be deaminated to restore the wild-type amino acid and confer fluorescence. PIK3CA mutation: a mutant codon encoding K111E mutant PIK3CA can be deaminated to restore the wild-type amino acid residue with applications in cancer. CTNNB1 mutation: a mutant codon encoding T41A mutant CTNNB1 can be deaminated to restore the wild-type amino acid residue with applications in cancer. HRAS mutation: a mutant codon encoding Q61R mutant HRAS can be deaminated to restore the wild-type amino acid residue with applications in cancer. P53 mutations: any of the mutant codons encoding Y163C, Y236C, or N239D mutant p53 can be deaminated to encode the wild type amino acid sequence with applications in cancer.

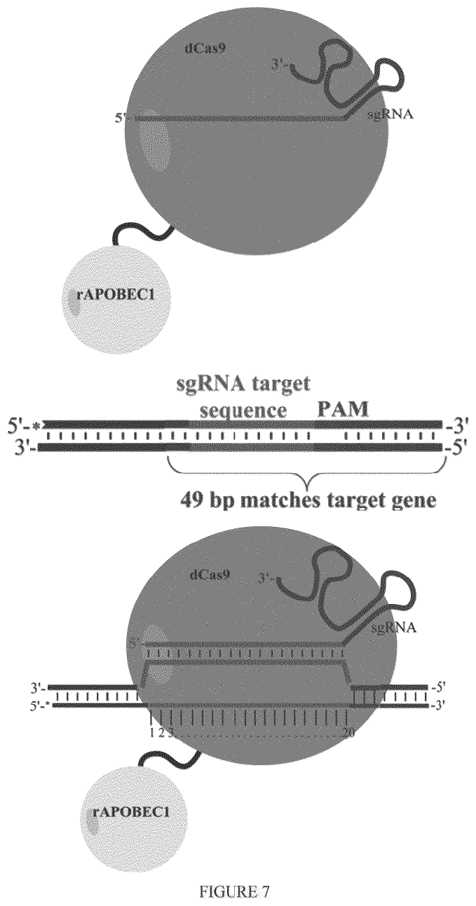

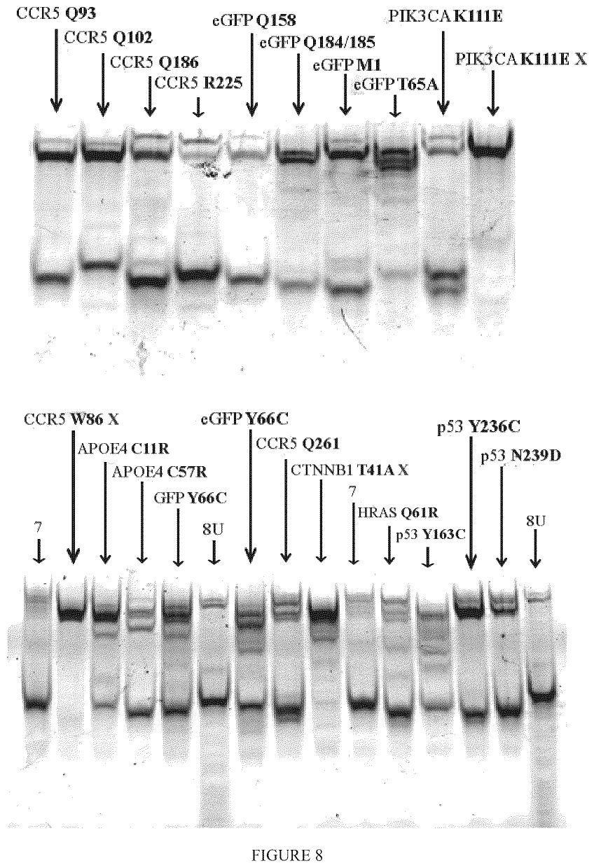

The feasibility of deaminating these target sequences in double-stranded DNA is demonstrated in FIGS. 7 and 8. FIG. 7 illustrates the mechanism of target DNA binding of in vivo target sequences by deaminase-dCas9:sgRNA complexes.

FIG. 8 shows successful deamination of exemplary disease-associated target sequences. Upper Gel: CCR5 Q93: coding strand target in pos. 10 (potential off-targets at positions 2, 5, 6, 8, 9); CCR5 Q102: coding strand target in pos. 9 (potential off-targets at positions 1, 12, 14); CCR5 Q186: coding strand target in pos. 9 (potential off-targets at positions 1, 5, 15); CCR5 R225: coding strand target in pos. 6 (no potential off-targets); eGFP Q158: coding strand target in pos. 5 (potential off-targets at positions 1, 13, 16); eGFP Q184/185: coding strand target in pos. 4 and 7 (potential off-targets at positions 3, 12, 14, 15, 16, 17, 18); eGFP M1: template strand target in pos. 12 (potential off-targets at positions 2, 3, 7, 9, 11) (targets positions 7 and 9 to small degree); eGFP T65A: template strand target in pos. 7 (potential off-targets at positions 1, 8, 17); PIK3CA K111E: template strand target in pos. 2 (potential off-targets at positions 5, 8, 10, 16, 17); PIK3CA K111E: template strand target in pos. 13 (potential off-targets at positions 11, 16, 19) X. Lower Gel: CCR5 W86: template strand target in pos. 2 and 3 (potential off-targets at positions 1, 13) X; APOE4 C11R: coding strand target in pos. 11 (potential off-targets at positions 7, 13, 16, 17); APOE4 C57R: coding strand target in pos. 5) (potential off-targets at positions 7, 8, 12); eGFP Y66C: template strand target in pos. 11 (potential off-targets at positions 1, 4, 6, 8, 9, 16); eGFP Y66C: template strand target in pos. 3 (potential off-targets at positions 1, 8, 17); CCR5 Q261: coding strand target in pos. 10 (potential off-targets at positions 3, 5, 6, 9, 18); CTNNB1 T41A: template strand target in pos. 7 (potential off-targets at positions 1, 13, 15, 16) X; HRAS Q61R: template strand target in pos. 6 (potential off-targets at positions 1, 2, 4, 5, 9, 10, 13); p53 Y163C: template strand target in pos. 6 (potential off-targets at positions 2, 13, 14); p53 Y236C: template strand target in pos. 8 (potential off-targets at positions 2, 4); p53 N239D: template strand target in pos. 4 (potential off-targets at positions 6, 8). Exemplary DNA sequences of disease targets are provided below (PAMs (5′-NGG-3′) and target positions are boxed):

(SEQ ID NO: 636)
CCR5 Q93: 5′-Cy3-GTAGGTAGTTAGGATGAATGGAAGGTTGGTAACTATGCTGCCGCC

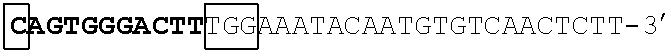

(SEQ ID NO: 637)
CCR5 Q102: 5′-Cy3-GTAGGTAGTTAGGATGAATGGAAGGTTGGTAAAATACAATGTGT

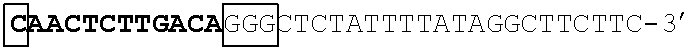

(SEQ ID NO: 638)
CCR5 Q186: 5′-Cy3-GTAGGTAGTTAGGATGAATGGAAGGTTGGTATTTTCCATACAGT

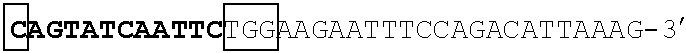

(SEQ ID NO: 639)

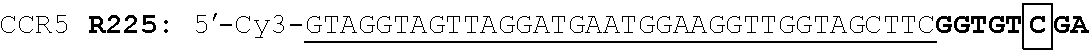

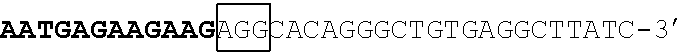

(SEQ ID NO: 640)
CCR5 W86: 5′-Cy3-

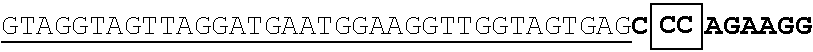

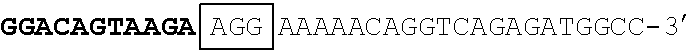

(SEQ ID NO: 641)
CCR5 Q261: 5′-Cy3-GTAGGTAGTTAGGATGAATGGAAGGTTGGTATCCTGAACACCTT

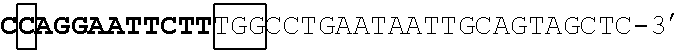

(SEQ ID NO: 642)
APOE4 C11R: 5′-Cy3-GTAGGTAGTTAGGATGAATGGAAGGTTGGTAGACATGGAGGAC

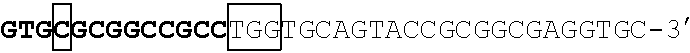

(SEQ ID NO: 643)

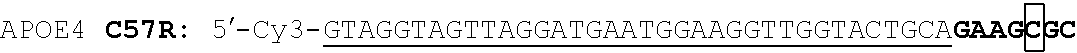

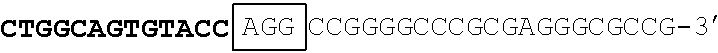

(SEQ ID NO: 644)

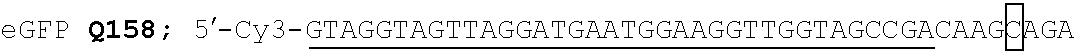

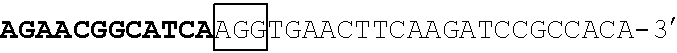

(SEQ ID NO: 645)

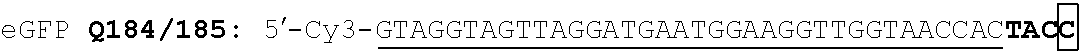

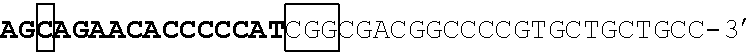

(SEQ ID NO: 646)
eGFP M1: 5′-Cy3-GTAGGTAGTTAGGATGAATGGAAGGTTGGTACCTCGCCCTTGCTCA

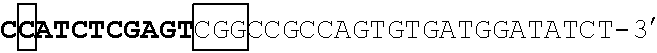

(SEQ ID NO: 647)
eGFP T65A: 5′-Cy3-GTAGGTAGTTAGGATGAATGGAAGGTTGGTACACGCGTAGGCCA 113

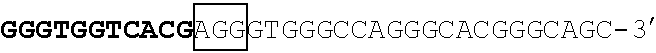

(SEQ ID NO: 648)
eGFP Y66C: 5′-Cy3-GTAGGTAGTTAGGATGAATGGAAGGTTGGTAAAGCACTGCACTC

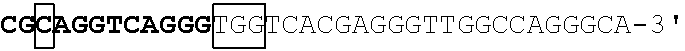

(SEQ ID NO: 649)

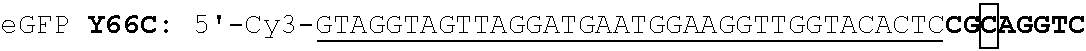

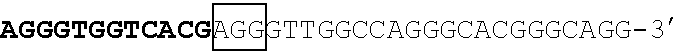

(SEQ ID NO: 650)

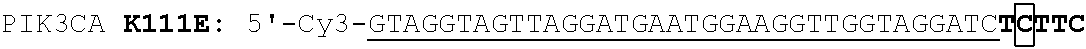

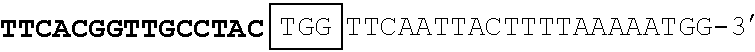

(SEQ ID NO: 651)
PIK3CA K111E: 5′-Cy3-GTAGGTAGTTAGGATGAATGGAAGGTTGGTATTCTCGATTG

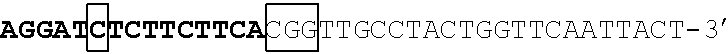

(SEQ ID NO: 652)
CTNNB1 T41A: 5′-Cy3-GTAGGTAGTTAGGATGAATGGAAGGTTGGTAAGGAGCTGTGG

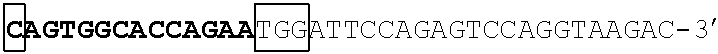

(SEQ ID NO: 653)

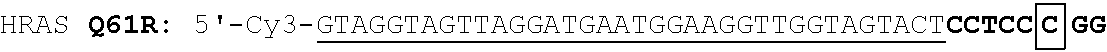

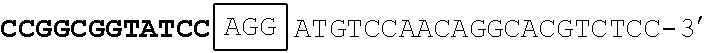

(SEQ ID NO: 654)

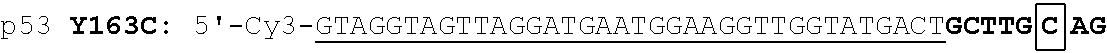

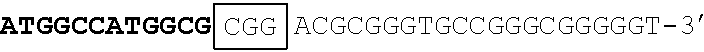

(SEQ ID NO: 655)

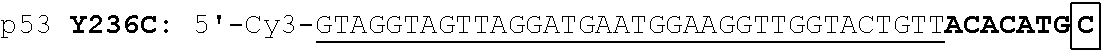

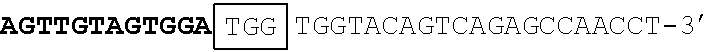

(SEQ ID NO: 656)

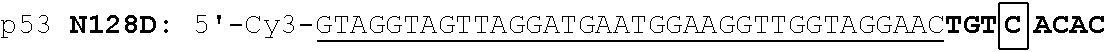

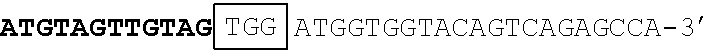

### Example 3: Uracil Glycosylase Inhibitor Fusion Improves Deamination Efficiency

Direct programmable nucleobase editing efficiencies in mammalian cells by dCas9:deaminase fusion proteins can be improved significantly by fusing a uracil glycosylase inhibitor (UGI) to the dCas9:deaminase fusion protein.

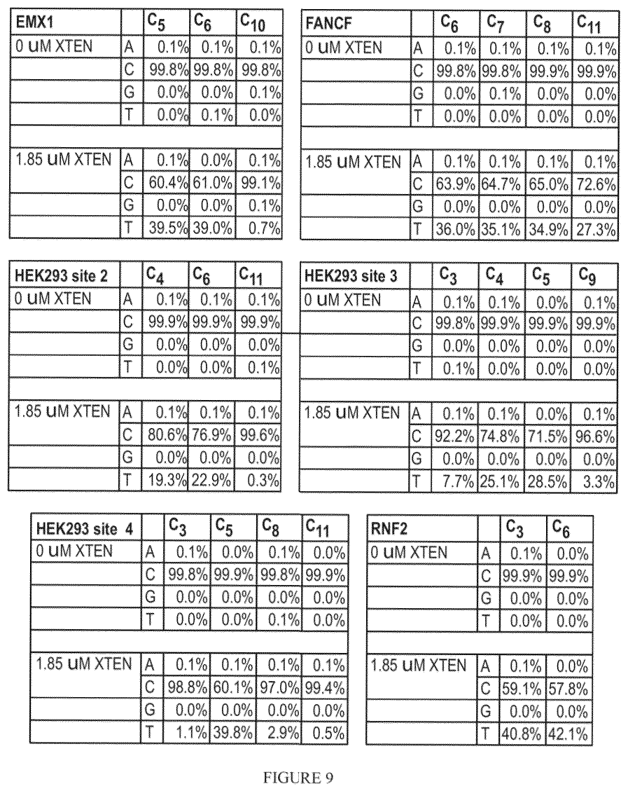

FIG. 9 shows in vitro C→T editing efficiencies in human HEK293 cells using rAPOBEC1-XTEN-dCas9:

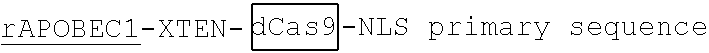

MSSETGPVAVDPTLRRRIEPHEFEVFFDPRELRKETCLLYEINWGGRHSIWRHTSONTNKH
VEVNFIEKFTTERYFCPNTRCSITWFLSWSPCGECSRAITEFLSRYPHVTLFIYIARLYHHAD
PRNRQGLRDLISSGVTIQIMTEQESGYCWRNFVNYSPSNEAHWPRYPHLWVRLYVLELYC

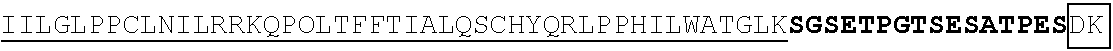

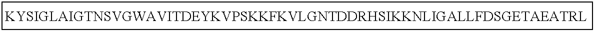

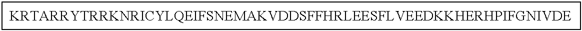

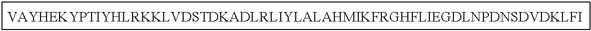

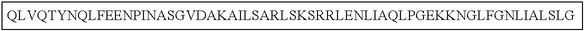

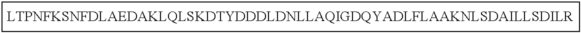

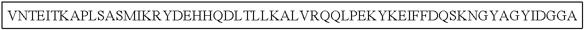

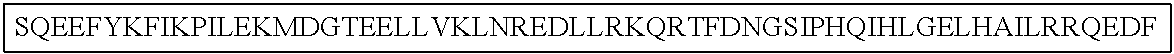

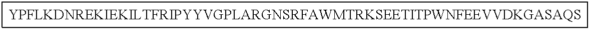

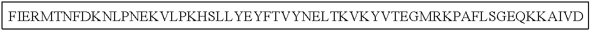

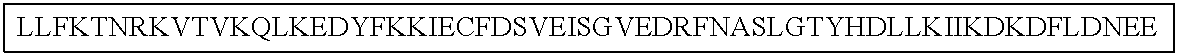

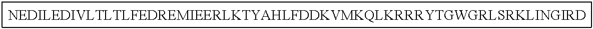

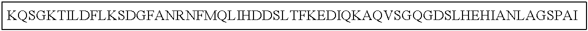

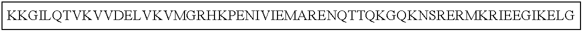

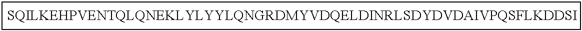

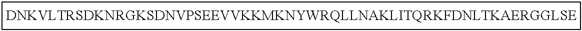

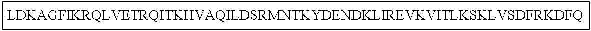

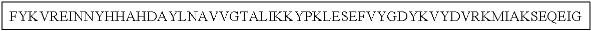

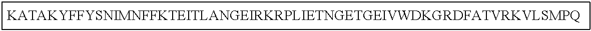

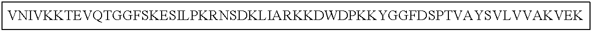

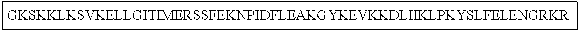

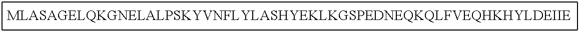

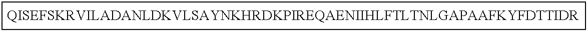

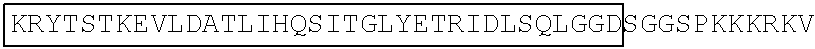

Protospacer sequences were as follows:

(SEQ ID NO: 293)
EMX1: 5′-GAGTC5C6GAGC10AGAAGAAGAA 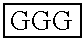 -3′
(SEQ ID NO: 294)
FANCF: 5′-GGAATC6C7C8TTC11TGCAGCACC 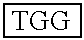 -3′
(SEQ ID NO: 295)
HEK293 site 2: 5′-GAAC4AC6AAAGC11ATAGACTGC 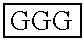 -3′
(SEQ ID NO: 296)
HEK293 site 3: 5′-GGC3C4C5AGAC9TGAGCACGTGA 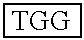 -3′
(SEQ ID NO: 297)
HEK293 site 4: 5′-GGC3AC5TGC8GGC11TGGAGGTGG 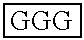 -3′
(SEQ ID NO: 298)
RNF2: 5′-GTC3ATC6TTAGTCATTACCTG 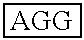 -3′
*PAMs are boxed, C residues within target window (positions 3-11) are
numbered and bolded.

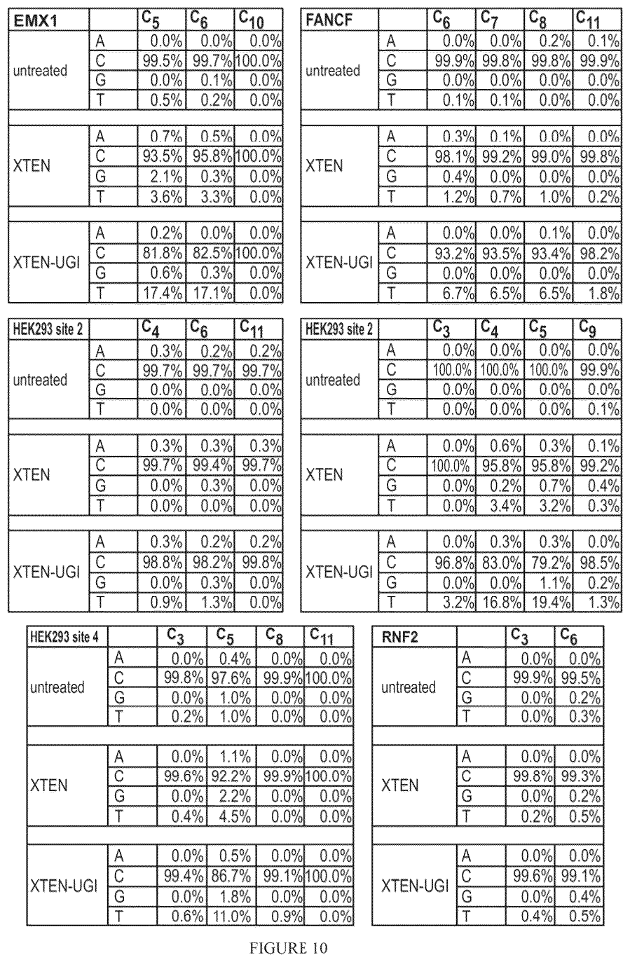

FIG. 10 demonstrates that C→T editing efficiencies on the same protospacer sequences in HEK293T cells are greatly enhanced when a UGI domain is fused to the rAPOBEC1:dCas9 fusion protein.

rAPOBEC1-XTEN- 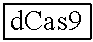 -UGI-NLS primary sequence
(SEQ ID NO: 658)
MSSETGPVAVDPTLRRRIEPHEFEVFFDPRELRKETCLLYEINWGGRHSIWRHTSONTNKH
VEVNFIEKFTTERYFCPNTRCSITWFLSWSPCGECSRAITEFLSRYPHVTLFIYIARLYHHAD
PRNRQGLRDLISSGVTIQIMTEQESGYCWRNFVNYSPSNEAHWPRYPHLWVRLYVLELYC

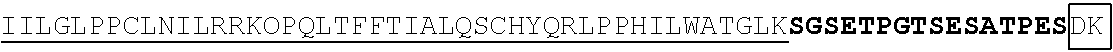

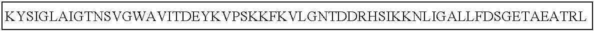

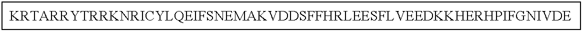

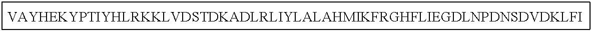

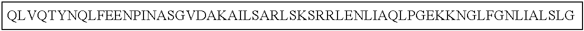

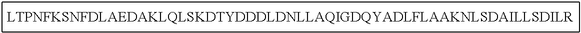

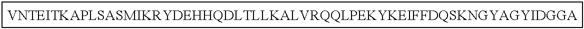

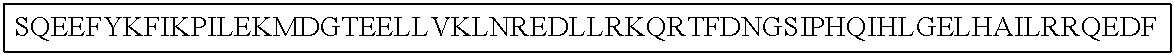

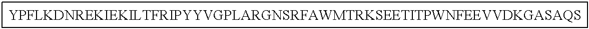

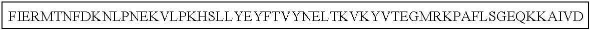

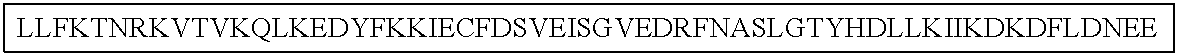

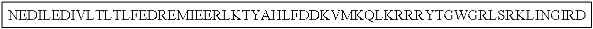

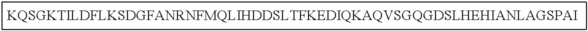

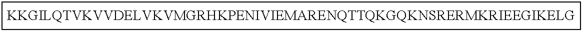

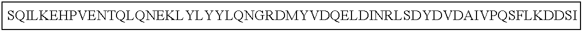

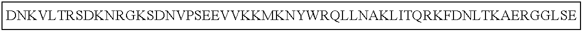

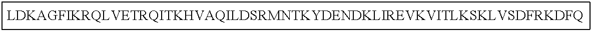

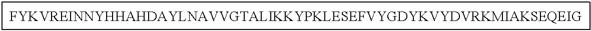

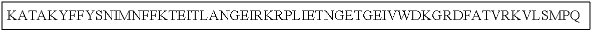

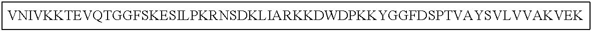

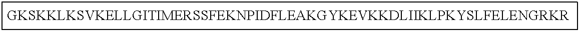

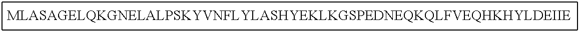

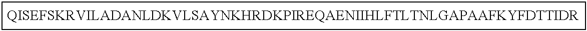

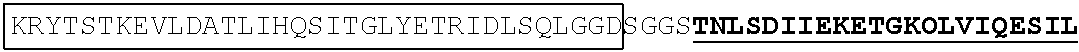

MLPEEVEEVIGNKPESDILVHTAYDESTDENVMLLTSDAPEYKPWALVIODSNGENKI
KMLSGGSPKKKRKV

The percentages in FIGS. 9 and 10 are shown from sequencing both strands of the target sequence. Because only one of the strands is a substrate for deamination, the maximum possible deamination value in this assay is 50%. Accordingly, the deamination efficiency is double the percentages shown in the tables. E.g., a value of 50% relates to deamination of 100% of double-stranded target sequences.

When a uracil glycosylase inhibitor (UGI) was fused to the dCas9:deaminase fusion protein (e.g., rAPOBEC1-XTEN-dCas9-[UGI]-NLS), a significant increase in editing efficiency in cells was observed. This result indicates that in mammalian cells, the DNA repair machinery that cuts out the uracil base in a U:G base pair is a rate-limiting process in DNA editing. Tethering UGI to the dVas9:deaminase fusion proteins greatly increases editing yields.

Without UGI, typical editing efficiencies in human cells were in the ˜2-14% yield range (FIG. 9 and FIG. 10, “XTEN” entries). With UGI (FIG. 10, “UGI” entries) the editing was observed in the ˜6-40% range. Using a UGI fusion is thus more efficient than the current alternative method of correcting point mutations via HDR, which also creates an excess of indels in addition to correcting the point mutation. No indels resulting from treatment with the cas9:deaminase:UGI fusions were observed.

### Example 4: Direct, Programmable Conversion of a Target Nucleotide in Genomic DNA without Double-Stranded DNA Cleavage

Current genome-editing technologies introduce double-stranded DNA breaks at a target locus of interest as the first step to gene correction.^{39,40} Although most genetic diseases arise from mutation of a single nucleobase to a different nucleobase, current approaches to revert such changes are very inefficient and typically induce an abundance of random insertions and deletions (indels) at the target locus as a consequence of the cellular response to double-stranded DNA breaks.^{39,40} Reported herein is the development of nucleobase editing, a new strategy for genome editing that enables the direct conversion of one target nucleobase into another in a programmable manner, without requiring double-stranded DNA backbone cleavage. Fusions of CRISPR/Cas9 were engineered and the cytidine deaminase enzyme APOBEC1 that retain the ability to be programmed with a guide RNA, do not induce double-stranded DNA breaks, and mediate the direct conversion of cytidine to uracil, thereby effecting a C→T (or G→A) substitution following DNA replication, DNA repair, or transcription if the template strand is targeted. The resulting “nucleobase editors” convert cytidines within a window of approximately five nucleotides, and can efficiently correct a variety of point mutations relevant to human disease in vitro. In four transformed human and murine cell lines, second- and third-generation nucleobase editors that fuse uracil glycosylase inhibitor (UGI), and that use a Cas9 nickase targeting the non-edited strand, respectively, can overcome the cellular DNA repair response to nucleobase editing, resulting in permanent correction of up to 37% or (˜15-75%) of total cellular DNA in human cells with minimal (typically <1%) indel formation. In contrast, canonical Cas9-mediated HDR on the same targets yielded an average of 0.7% correction with 4% indel formation. Nucleobase editors were used to revert two oncogenic p53 mutations into wild-type alleles in human breast cancer and lymphoma cells, and to convert an Alzheimer's Disease associated Arg codon in ApoE4 into a non-disease-associated Cys codon in mouse astrocytes. Base editing expands the scope and efficiency of genome editing of point mutations.

The clustered regularly interspaced short palindromic repeat (CRISPR) system is a prokaryotic adaptive immune system that has been adapted to mediate genome engineering in a variety of organisms and cell lines.⁴¹ CRISPR/Cas9 protein-RNA complexes localize to a target DNA sequence through base pairing with a guide RNA, and natively create a DNA double-stranded break (DSB) at the locus specified by the guide RNA. In response to DSBs, endogenous DNA repair processes mostly result in random insertions or deletions (indels) at the site of DNA cleavage through non-homologous end joining (NHEJ). In the presence of a homologous DNA template, the DNA surrounding the cleavage site can be replaced through homology-directed repair (HDR). When simple disruption of a disease-associated gene is sufficient (for example, to treat some gain-of-function diseases), targeted DNA cleavage followed by indel formation can be effective. For most known genetic diseases, however, correction of a point mutation in the target locus, rather than stochastic disruption of the gene, is needed to address or study the underlying cause of the disease.⁶⁸

Motivated by this need, researchers have invested intense effort to increase the efficiency of HDR and suppress NHEJ. For example, a small-molecule inhibitor of ligase IV, an essential enzyme in the NHEJ pathway, has been shown to increase HDR efficiency.^{42,43} However, this strategy is challenging in post-mitotic cells, which typically down-regulate HDR, and its therapeutic relevance is limited by the potential risks of inhibiting ligase IV in non-target cells. Enhanced HDR efficiency can also be achieved by the timed delivery of Cas9-guide RNA complexes into chemically synchronized cells, as HDR efficiency is highly cell-cycle dependent.⁴⁴ Such an approach, however, is limited to research applications in cell culture since synchronizing cells is highly disruptive. Despite these developments, current strategies to replace point mutations using HDR in most contexts are very inefficient (typically ˜0.1 to 5%),^{42,43,45,46, 75} especially in unmodified, non-dividing cells. In addition, HDR competes with NHEJ during the resolution of double-stranded breaks, and indels are generally more abundant outcomes than gene replacement. These observations highlight the need to develop alternative approaches to install specific modifications in genomic DNA that do not rely on creating double-stranded DNA breaks. A small-molecule inhibitor of ligase IV, an essential enzyme in the NHEJ pathway, has been shown to increase HDR efficiency.^{42,43} However, this strategy is challenging in post-mitotic cells, which typically down-regulate HDR, and its therapeutic relevance is limited by the potential risks of inhibiting ligase IV in non-target cells. Enhanced HDR efficiency can also be achieved by the timed delivery of Cas9-guide RNA complexes into chemically synchronized cells, as HDR efficiency is highly cell-cycle dependent.⁴⁴ Such an approach, however, is limited to research applications in cell culture since synchronizing cells is highly disruptive. In some cases, it is possible to design HDR templates such that the product of successful HDR contains mutations in the PAM sequence and therefore is no longer a substrate for subsequent Cas9 modification, increasing the overall yield of HDR products,⁷⁵ although such an approach imposes constraints on the product sequences. Recently, this strategy has been coupled to the use of ssDNA donors that are complementary to the non-target strand and high-efficiency ribonucleoprotein (RNP) delivery to substantially increase the efficiency of HDR, but even in these cases the ratio of HDR to NHEJ outcomes is relatively low (<2).⁸³

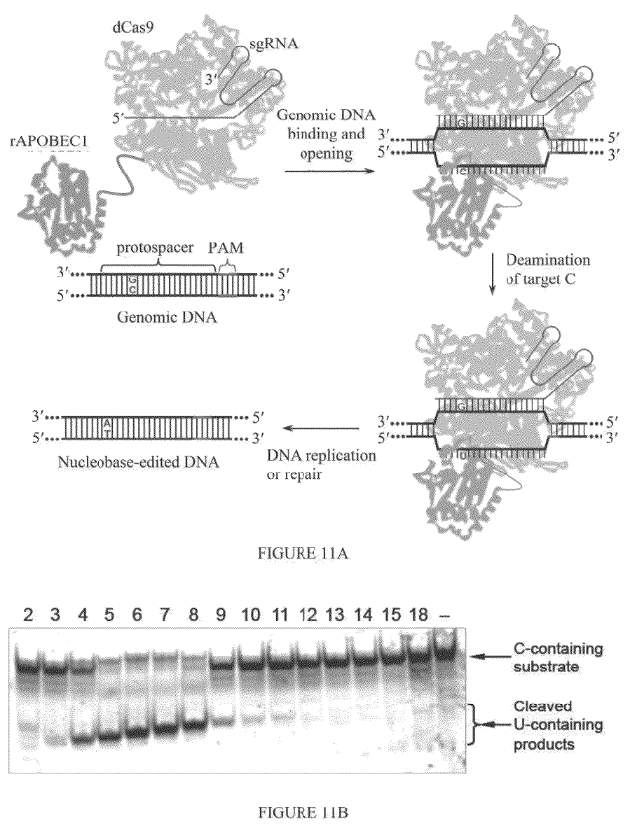

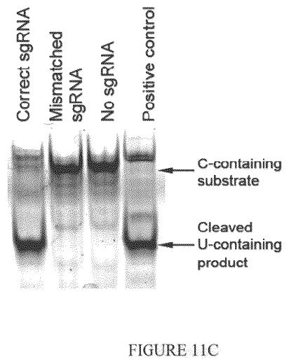

It was envisioned that direct catalysis of the conversion of one nucleobase to another at a programmable target locus without requiring DNA backbone cleavage could increase the efficiency of gene correction relative to HDR without introducing undesired random indels at the locus of interest. Catalytically dead Cas9 (dCas9), which contains Asp10A1a and His840Ala mutations that inactivate its nuclease activity, retains its ability to bind DNA in a guide RNA-programmed manner but does not cleave the DNA backbone.^{16,47} In principle, conjugation of dCas9 with an enzymatic or chemical catalyst that mediates the direct conversion of one nucleobase to another could enable RNA-programmed nucleobase editing. The deamination of cytosine (C) is catalyzed by cytidine deaminases²⁹ and results in uracil (U), which has the base pairing properties of thymine (T). dCas9 was fused to cytidine deaminase enzymes in order to test their ability to convert C to U at a guide RNA-specified DNA locus. Most known cytidine deaminases operate on RNA, and the few examples that are known to accept DNA require single-stranded DNA.⁴⁸ Recent studies on the dCas9-target DNA complex reveal that at least nine nucleotides of the displaced DNA strand are unpaired upon formation of the Cas9:guide RNA:DNA “R-loop” complex.¹² Indeed, in the structure of the Cas9 R-loop complex the first 11 nucleotides of the protospacer on the displaced DNA strand are disordered, suggesting that their movement is not highly restricted.⁷⁶ It has also been speculated that Cas9 nickase-induced mutations at cytosines in the non-template strand might arise from their accessibility by cellular cytidine deaminase enzymes.⁷⁷ Recent studies on the dCas9-target DNA complex have revealed that at least 26 bases on the non-template strand are unpaired when Cas9 binds to its target DNA sequence.⁴⁹ It was reasoned that a subset of this stretch of single-stranded DNA in the R-loop might serve as a substrate for a dCas9-tethered cytidine deaminase to effect direct, programmable conversion of C to U in DNA (FIG. 11A).

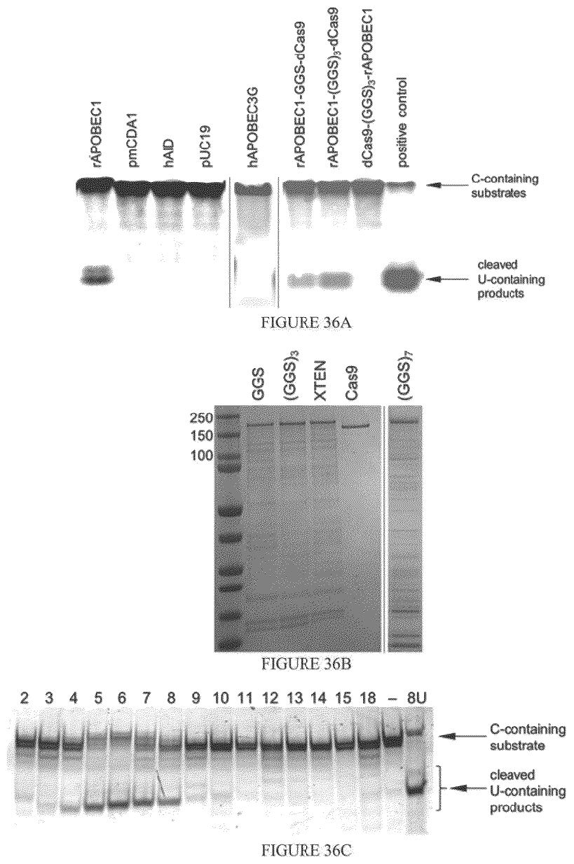

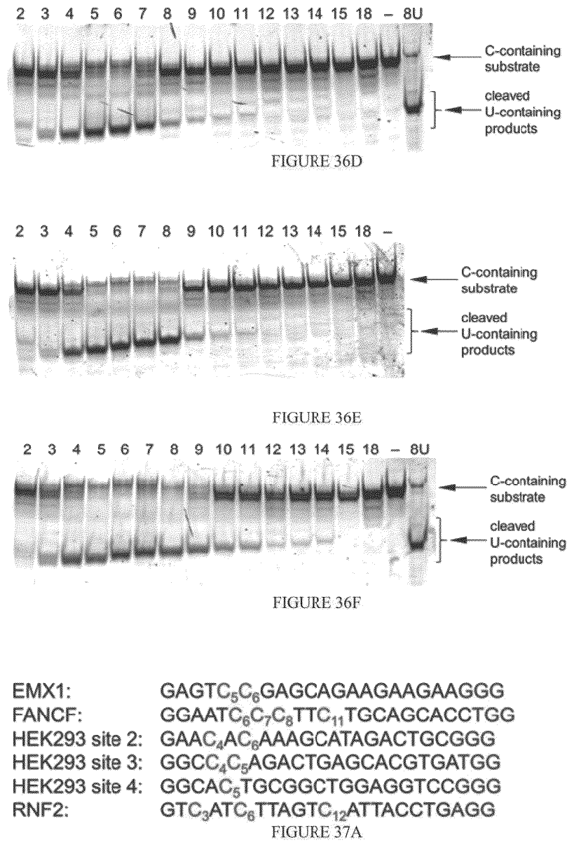

Four different cytidine deaminase enzymes (hAID, hAPOBEC3G, rAPOBEC1, and pmCDA1) were expressed in a mammalian cell lysate-derived in vitro transcription-translation system and evaluated for ssDNA deamination. Of the four enzymes, rAPOBEC1 showed the highest deaminase activity under the tested conditions and was chosen for dCas9 fusion experiments (FIG. 36A). Although appending rAPOBEC1 to the C-terminus of dCas9 abolishes deaminase activity, fusion to the N-terminus of dCas9 preserves deaminase activity on ssDNA at a level comparable to that of the unfused enzyme. Four rAPOBEC1-dCas9 fusions were expressed and purified with linkers of different length and composition (FIG. 36B), and evaluated each fusion for single guide RNA (sgRNA)-programmed dsDNA deamination in vitro (FIGS. 11A to 11C and FIGS. 15A to 15D).

Efficient, sequence-specific, sgRNA-dependent C to U conversion was observed in vitro (FIGS. 11A to 11C). Conversion efficiency was greatest using rAPOBEC1-dCas9 linkers over nine amino acids in length. The number of positions susceptible to deamination (the deamination “activity window”) increases with linker length was extended from three to 21 amino acids (FIGS. 36C to 36F15A to 15D). The 16-residue XTEN linker⁵⁰ was found to offer a promising balance between these two characteristics, with an efficient deamination window of approximately five nucleotides, from positions 4 to 8 within the protospacer, counting the end distal to the protospacer-adjacent motif (PAM) as position 1. The rAPOBEC1-XTEN-dCas9 protein served as the first-generation nucleobase editor (NBE1).

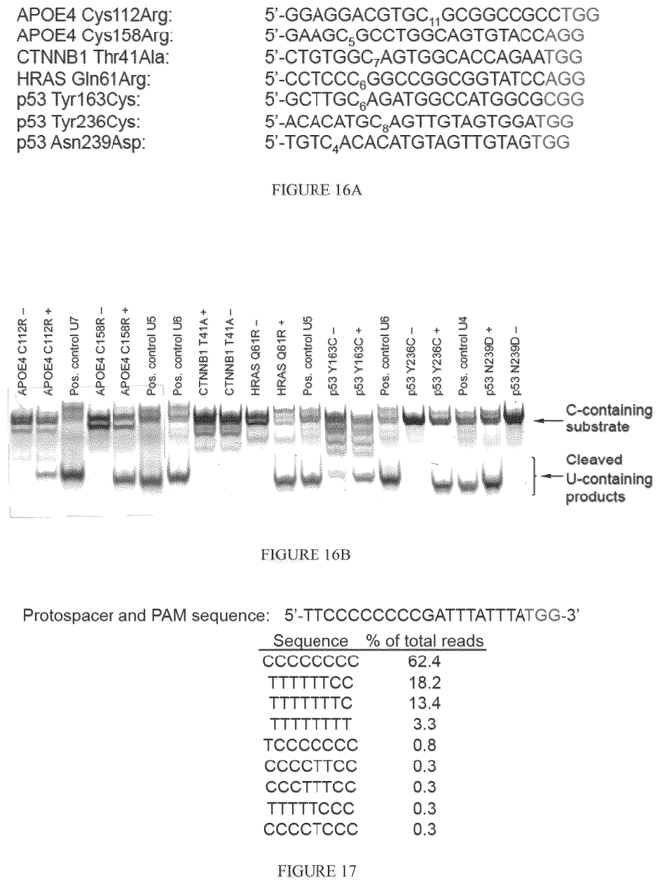

Elected were seven mutations relevant to human disease that in theory could be corrected by C to T nucleobase editing, synthesized double-stranded DNA 80-mers of the corresponding sequences, and assessed the ability of NBE1 to correct these mutations in vitro (FIGS. 16A to 16B). NBE1 yielded products consistent with efficient editing of the target C, or of at least one C within the activity window when multiple Cs were present, in six of these seven targets in vitro, with an average apparent editing efficiency of 44% (FIGS. 16A to 16B). In the three cases in which multiple Cs were present within the deamination window, evidence of deamination of some or all of these cytosines was observed. In only one of the seven cases tested were substantial yields of edited product observed (FIGS. 16A to 16B).

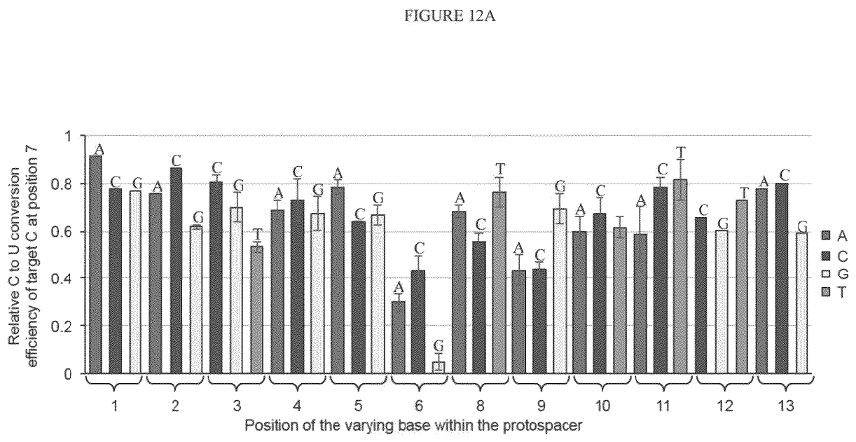

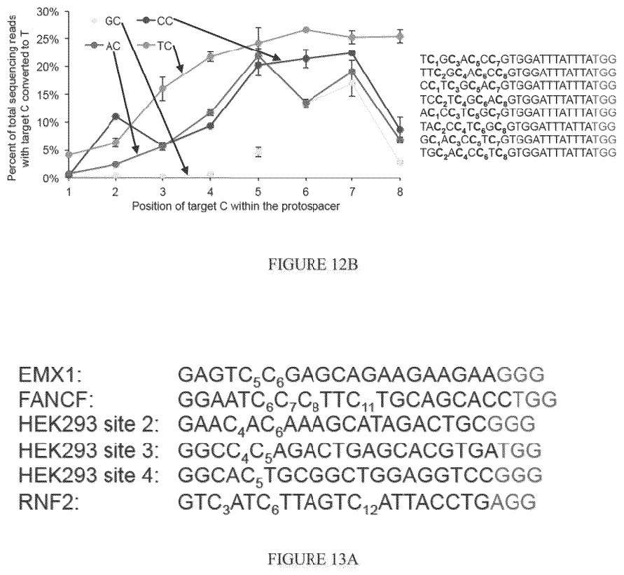

Although the preferred sequence context for APOBEC1 substrates is reported to be CC or TC,⁵¹ it was anticipated that the increased effective molarity of the deaminase and its single-stranded DNA substrate mediated by dCas9 binding to the target locus may relax this restriction. To illuminate the sequence context generality of NBE1, its ability to edit a 60-mer double-stranded DNA oligonucleotide containing a single fixed C at position 7 within the protospacer was assayed, as well as all 36 singly mutated variants in which protospacer bases 1-6 and 8-13 were individually varied to each of the other three bases. Each of these 37 sequences were treated with 1.9 μM NBE1, 1.9 μM of the corresponding sgRNA, and 125 nM DNA for 2 h, similar to standard conditions for in vitro Cas9 assays⁵². High-throughput DNA sequencing (HTS) revealed 50 to 80% C to U conversion of targeted strands (25 to 40% of total sequence reads arising from both DNA strands, one of which is not a substrate for NBE1) (FIG. 12A). The nucleotides surrounding the target C had little effect on editing efficiency was independent of sequence context unless the base immediately 5′ of the target C is a G, in which case editing efficiency was substantially lower (FIGS. 12A to 12B). NBE1 activity in vitro was assessed on all four NC motifs at positions 1 through 8 within the protospacer (FIGS. 12A to 12B). In general NBE1 activity on substrates was observed to follow the order TC≥CC≥AC≥GC, with maximum editing efficiency achieved when the target C is at or near position 7. In addition, it was observed that the nucleobase editor is highly processive, and will efficiently convert most of all Cs to Us on the same DNA strand within the 5-base activity window (FIG. 17).

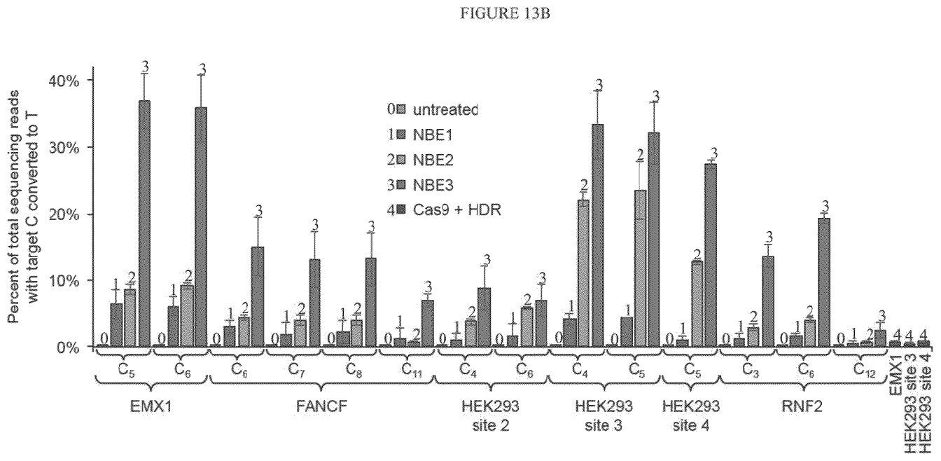

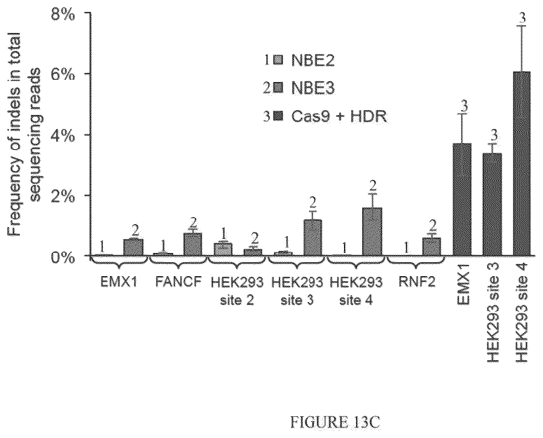

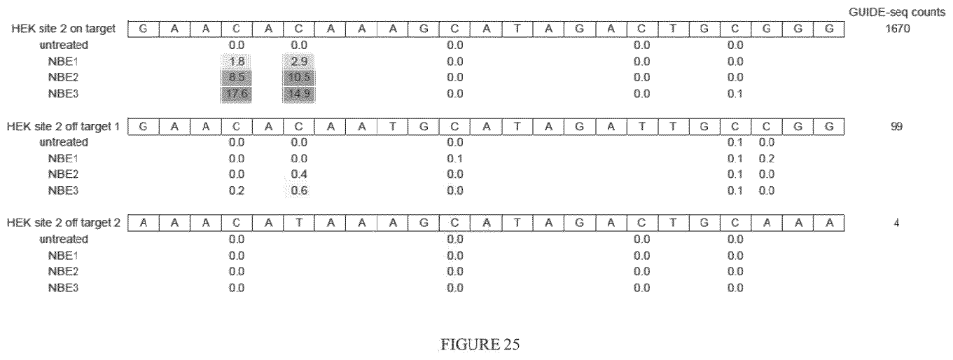

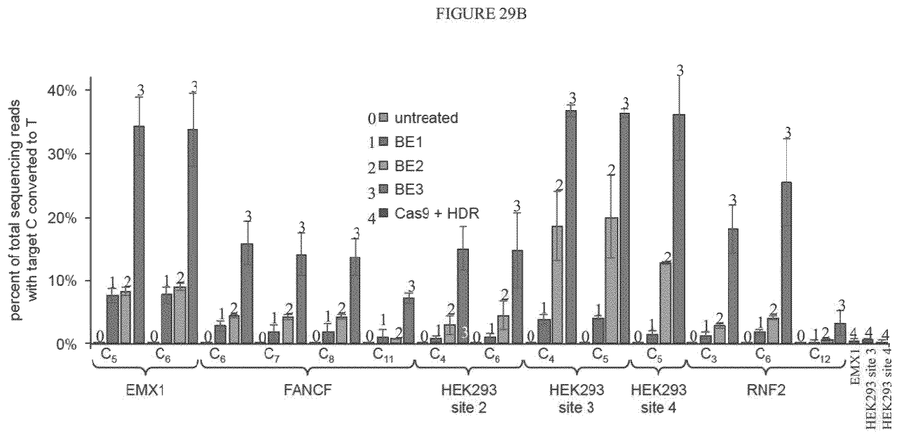

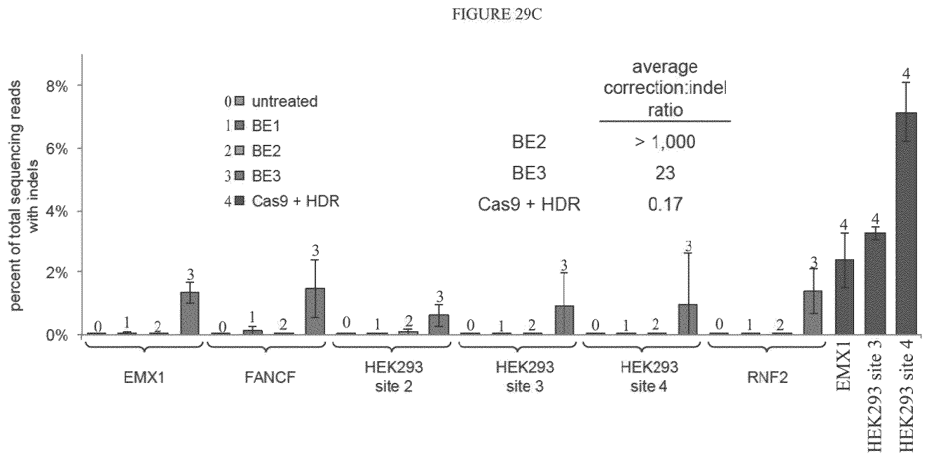

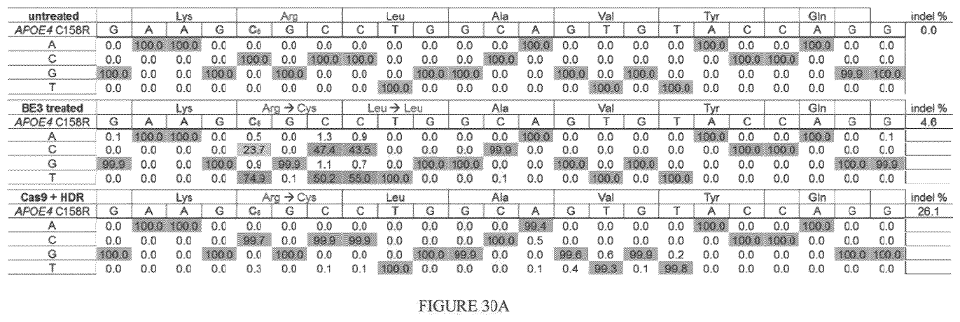

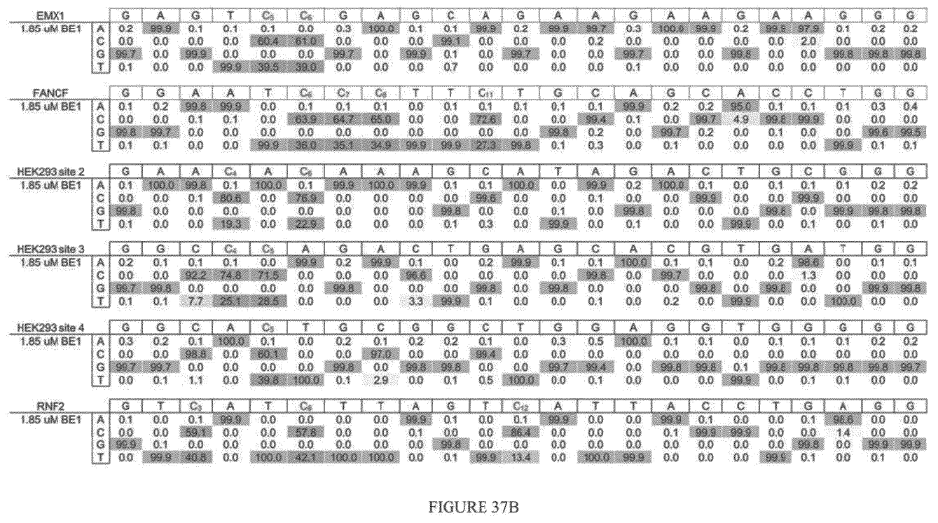

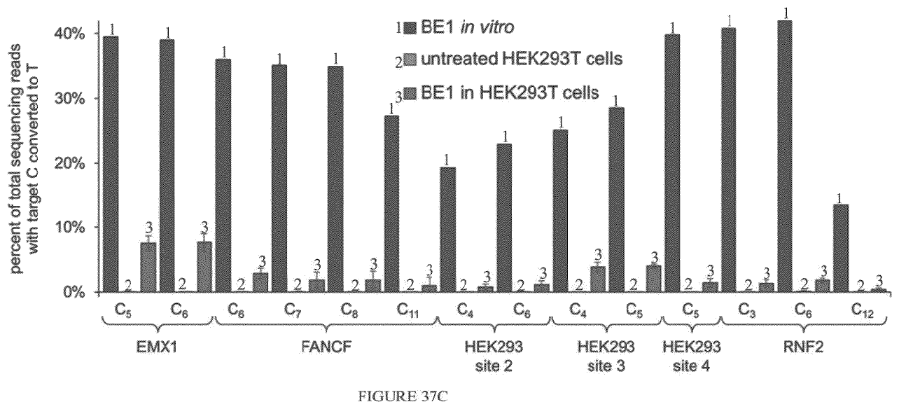

While BE1 efficiently processes substrates in a test tube, in cells a tree of possible DNA repair outcomes determines the fate of the initial U:G product of base editing (FIG. 29A). To test the effectiveness of nucleobase editing in human cells, NBE1 codon usage was optimized for mammalian expression, appended a C-terminal nuclear localization sequence (NLS),⁵³ and assayed its ability to convert C to T in human cells on 14Cs in six well-studied target sites throughout the human genome (FIG. 37A).⁵⁴ The editable Cs were confirmed within each protospacer in vitro by incubating NBE1 with synthetic 80-mers that correspond to the six different genomic sites, followed by HTS (FIGS. 13A to 13C, FIG. 29B and FIG. 25). Next, HEK293T cells were transfected with plasmids encoding NBE1 and one of the six target sgRNAs, allowed three days for nucleobase editing to occur, extracted genomic DNA from the cells, and analyzed the loci by HTS. Although C to T editing in cells at the target locus was observed for all six cases, the efficiency of nucleobase editing was 1.1% to 6.3% or 0.8%-7.7% of total DNA sequences (corresponding to 2.2% to 12.6% of targeted strands), a 6.3-fold to 37-fold or 5-fold to 36-fold decrease in efficiency compared to that of in vitro nucleobase editing (FIGS. 13A to 13C, FIG. 29B and FIG. 25). It was observed that some base editing outside of the typical window of positions 4 to 8 when the substrate C is preceded by a T, which we attribute to the unusually high activity of APOBEC1 for TC substrates.⁴⁸

It was asked whether the cellular DNA repair response to the presence of U:G heteroduplex DNA was responsible for the large decrease in nucleobase editing efficiency in cells (FIG. 29A). Uracil DNA glycosylase (UDG) catalyzes removal of U from DNA in cells and initiates base excision repair (BER), with reversion of the U:G pair to a C:G pair as the most common outcome (FIG. 29A).⁵⁵ Uracil DNA glycosylase inhibitor (UGI), an 83-residue protein from B. subtilis bacteriophage PBS1, potently blocks human UDG activity (IC₅₀=12 μM).⁵⁶ UGI was fused to the C-terminus of NBE1 to create the second-generation nucleobase editor NBE2 and repeated editing assays on all six genomic loci. Editing efficiencies in human cells were on average 3-fold higher with NBE2 than with NBE1, resulting in gene conversion efficiencies of up to 22.8% of total DNA sequenced (up to 45.6% of targeted strands) (FIGS. 13A to 13C and FIG. 29B). To test base editing in human cells, BE1 codon usage was optimized for mammalian expression and appended a C-terminal nuclear localization sequence (NLS).⁵³

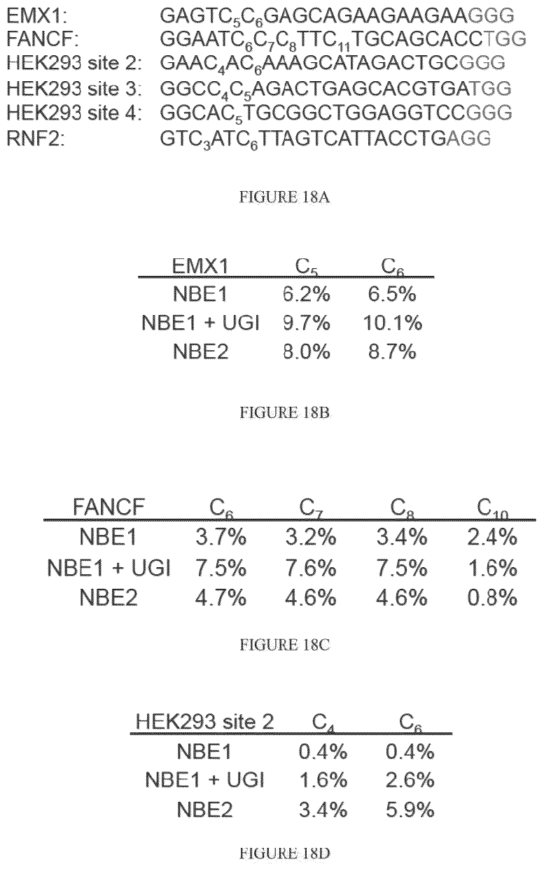

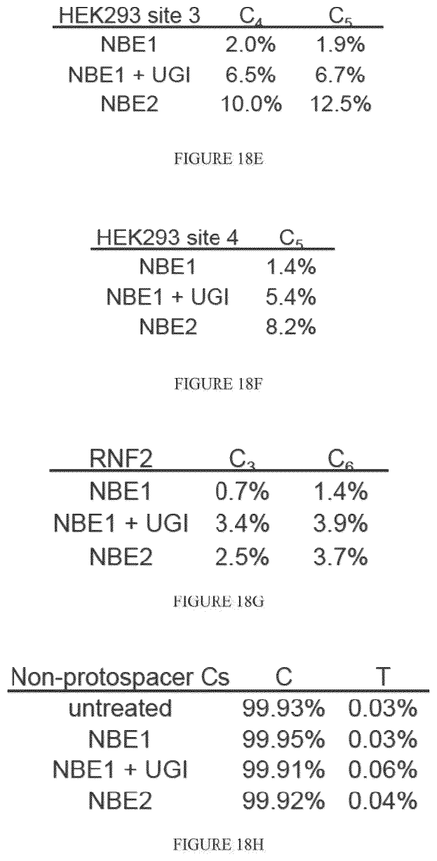

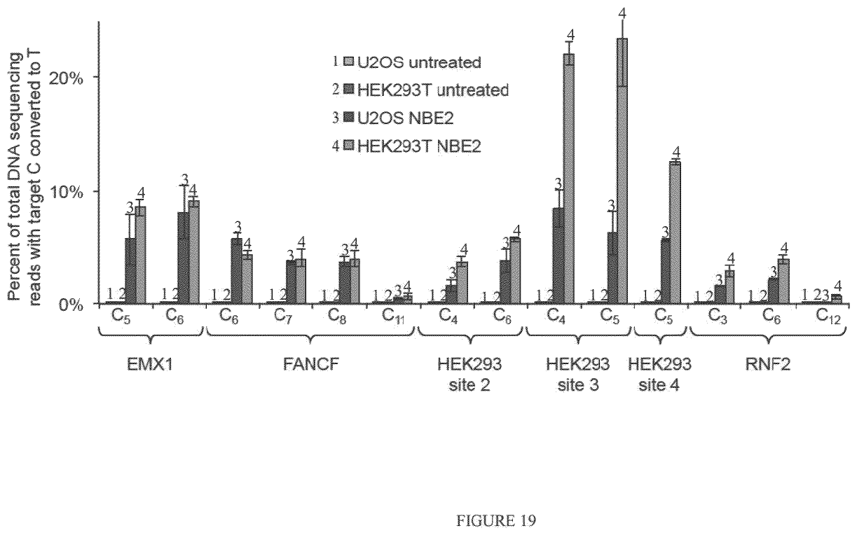

Similar editing efficiencies were observed when a separate plasmid overexpressing UGI was co-transfected with NBE1 (FIGS. 18A to 18H). However, while the direct fusion of UGI to NBE1 resulted in no significant increase in C to T mutations at monitored non-targeted genomic locations, overexpression of unfused UGI detectably increased the frequency of C to T mutations elsewhere in the genome (FIGS. 18A to 18H). The generality of NBE2-mediated nucleobase editing was confirmed by assessing editing efficiencies on the same six genomic targets in U2OS cells, and observed similar results with those in HEK293T cells (FIG. 19). Importantly, NBE2 typically did not result in any detectable indels (FIG. 13C and FIG. 29C), consistent with the known mechanistic dependence of NHEJ on double-stranded DNA breaks.^{57,78} Together, these results indicate that conjugating UGI to NBE1 can greatly increase the efficiency of nucleobase editing in human cells.

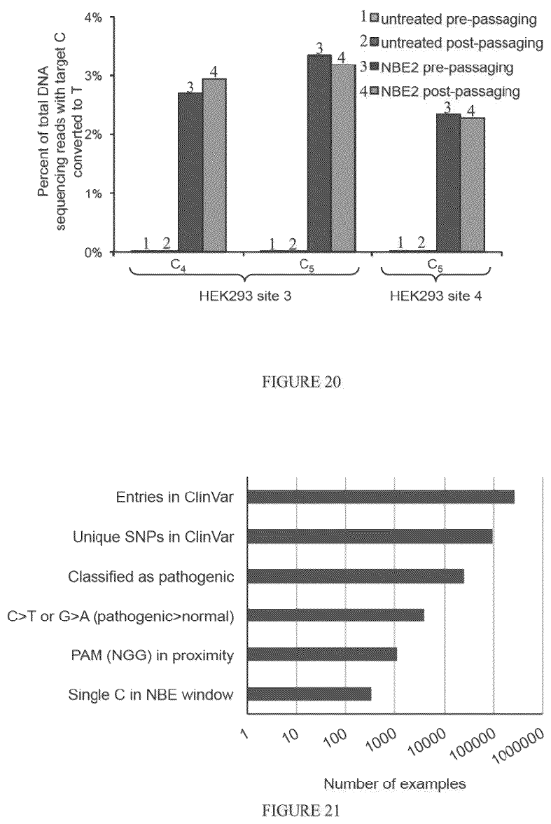

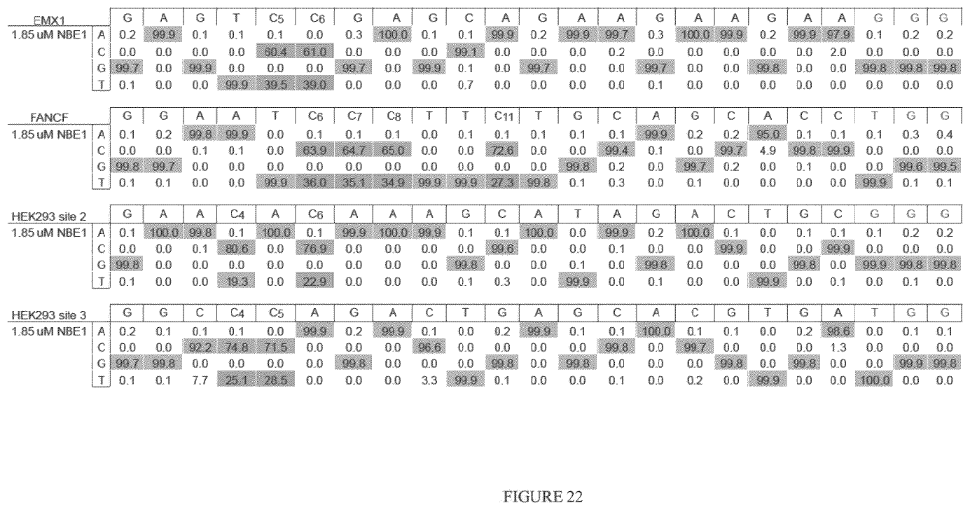

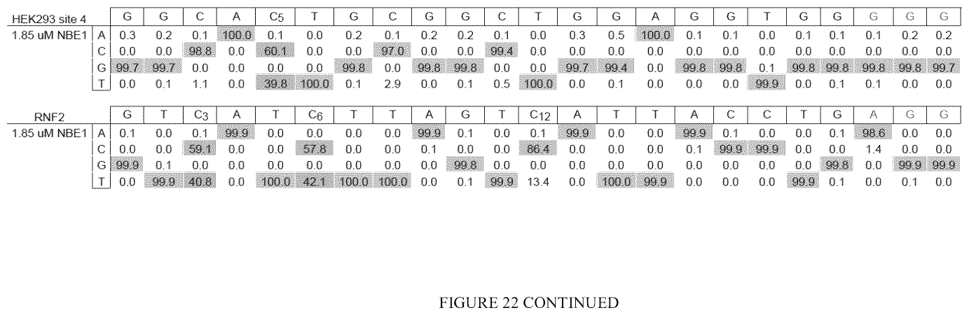

The permanence of nucleobase editing in human cells was confirmed by monitoring editing efficiencies over multiple cell divisions in HEK293T cells at two of the tested genomic loci. Genomic DNA was harvested at two time points: three days after transfection with plasmids expressing NBE2 and appropriate sgRNAs, and after passaging the cells and growing them for four additional days (approximately five subsequent cell divisions). No significant change in editing efficiency was observed between the non-passaged cells (editing observed in 4.6% to 6.6% of targeted strands for three different target Cs) and passaged cells (editing observed in 4.6% to 6.4% of targeted strands for the same three target Cs), confirming that the nucleobase edits became permanent following cell division (FIG. 20). Indels will on rare occasion arise from the processing of U:G lesions by cellular repair processes, which involve single-strand break intermediates that are known to lead to indels.⁸⁴ Given that several hundred endogenous U:G lesions are generated every day per human cell from spontaneous cytidine deaminase,⁸⁵ it was anticipate that the total indel frequency from U:G lesion repair is unlikely to increase from BE1 or BE2 activity at a single target locus.

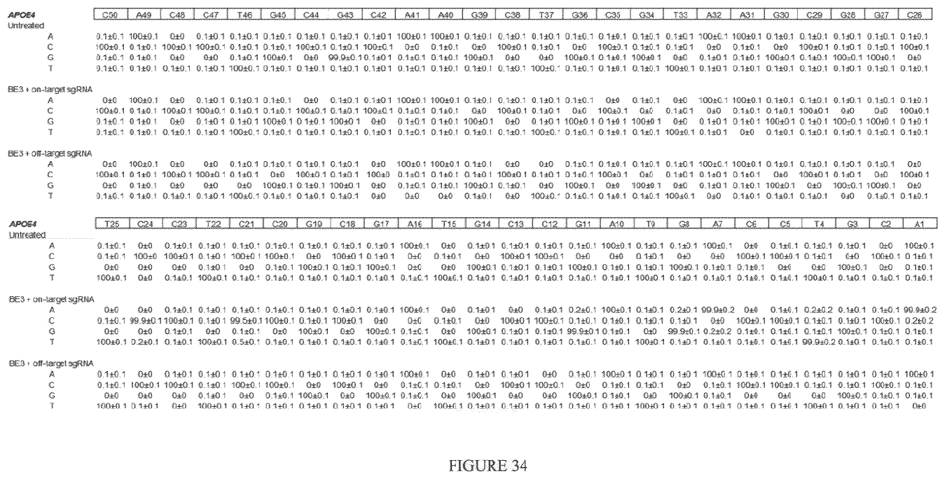

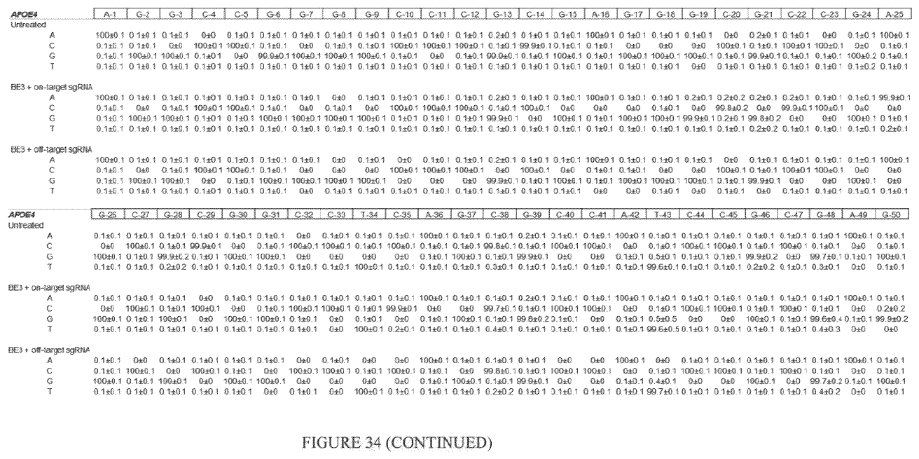

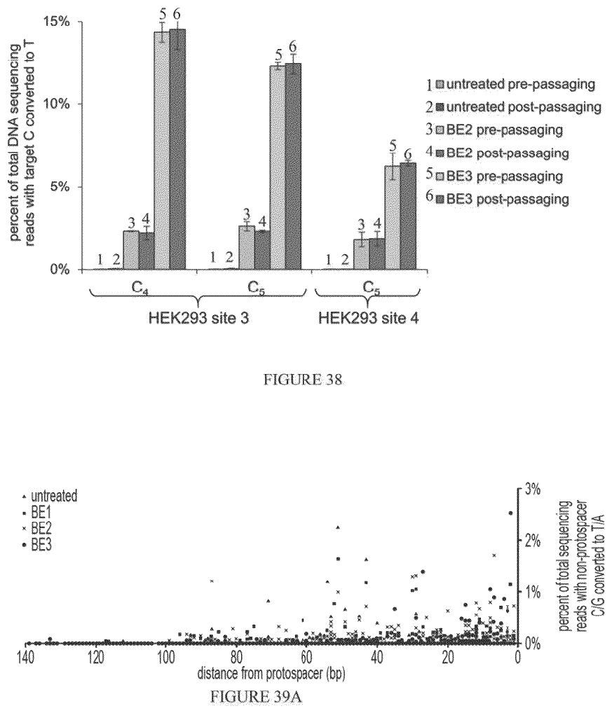

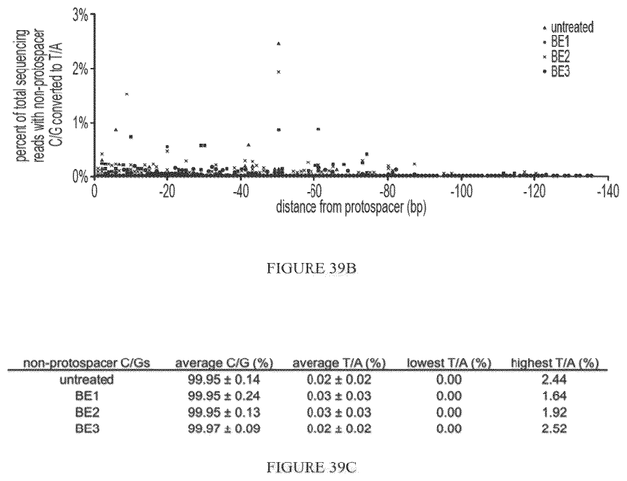

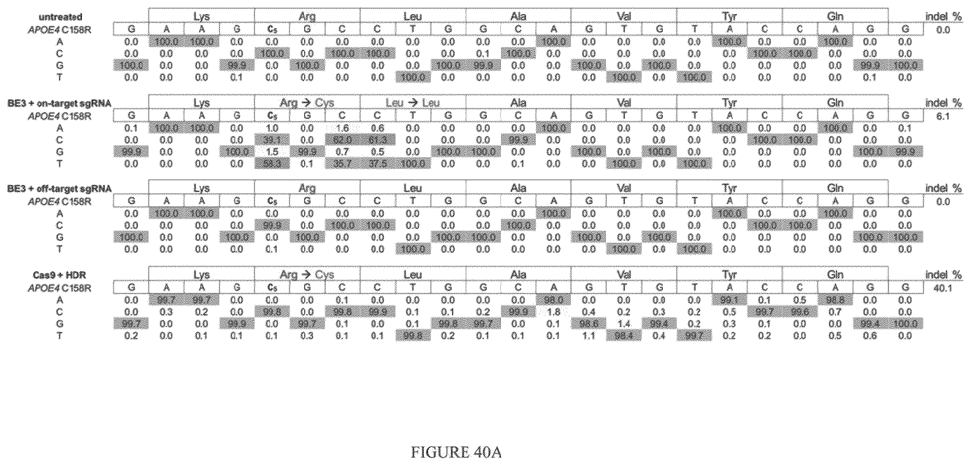

To further increase the efficiency of nucleobase editing in cells, it was anticipated that nicking the non-edited strand may result in a smaller fraction of edited Us being removed by the cell, since eukaryotic mismatch repair machinery uses strand discontinuity to direct DNA repair to any broken strand of a mismatched duplex (FIG. 29A).⁵⁸⁻⁷⁹⁻⁸⁰ The catalytic His residue was restored at position 840 in the Cas9 HNH domain,^{47,59} resulting in the third-generation nucleobase editor NBE3 that nicks the non-edited strand containing a G opposite the targeted C, but does not cleave the target strand containing the C. Because NBE3 still contains the Asp10A1a mutation in Cas9, it does not induce double-stranded DNA cleavage. This strategy of nicking the non-edited strand augmented nucleobase editing efficiency in human cells by an additional 1.4- to 4.8-fold relative to NBE2, resulting in up to 36.3% of total DNA sequences containing the targeted C to T conversion on the same six human genomic targets in HEK293T cells (FIGS. 13A to 13C and FIG. 29B). Importantly, only a small frequency of indels, averaging 0.8% (ranging from 0.2% to 1.6% for the six different loci), was observed from NBE3 treatment (FIG. 13C, FIG. 29C, and FIG. 34). In contrast, when cells were treated with wild-type Cas9, sgRNA, and a single-stranded DNA donor template to mediate HDR at three of these loci C to T conversion efficiencies averaging only 0.7% were observed, with much higher relative indel formation averaging 3.9% (FIGS. 13A to 13C and FIG. 29C). The ratio of allele conversion to NHEJ outcomes averaged>1,000 for BE2, 23 for BE3, and 0.17 for wild-type Cas9 (FIG. 3c). We confirmed the permanence of base editing in human cells by monitoring editing efficiencies over multiple cell divisions in HEK293T cells at the HEK293 site 3 and 4 genomic loci (FIG. 38). These results collectively establish that nucleobase editing can effect much more efficient targeted single-base editing in human cells than Cas9-mediated HDR, and with much less (NBE3) or no (NBE2) indel formation.

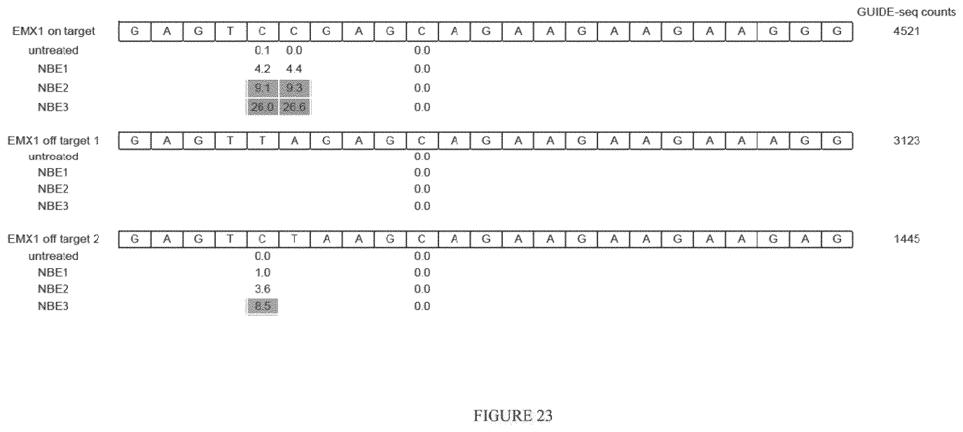

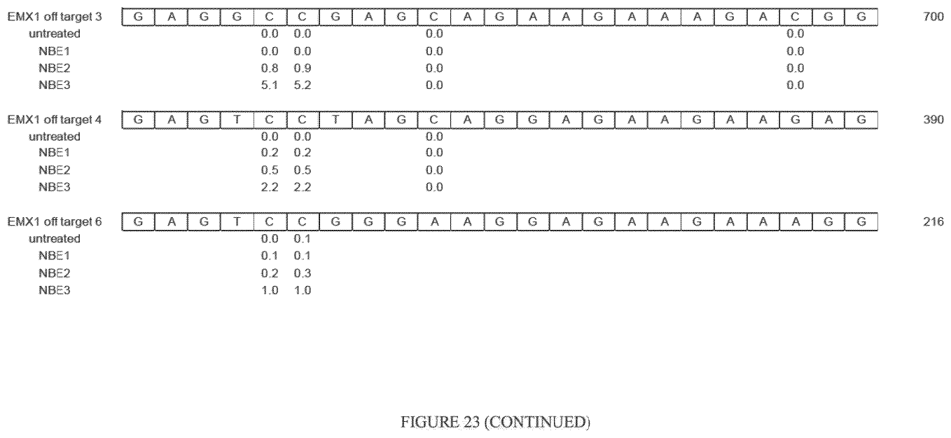

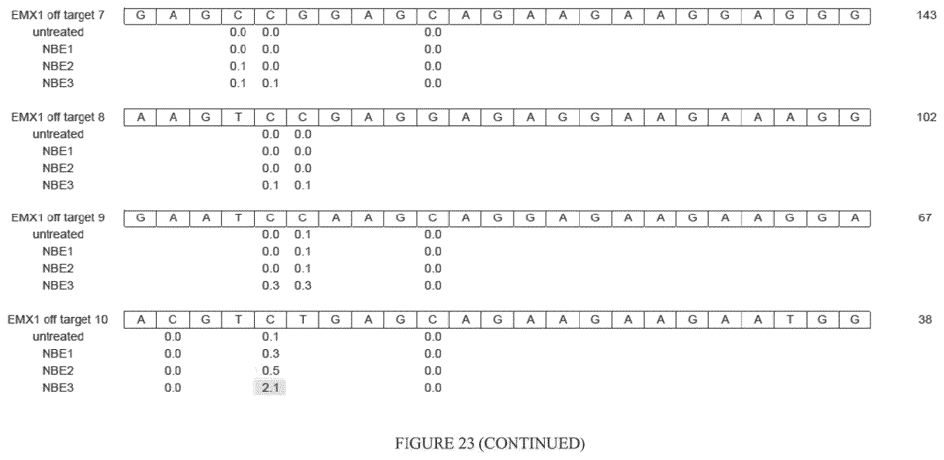

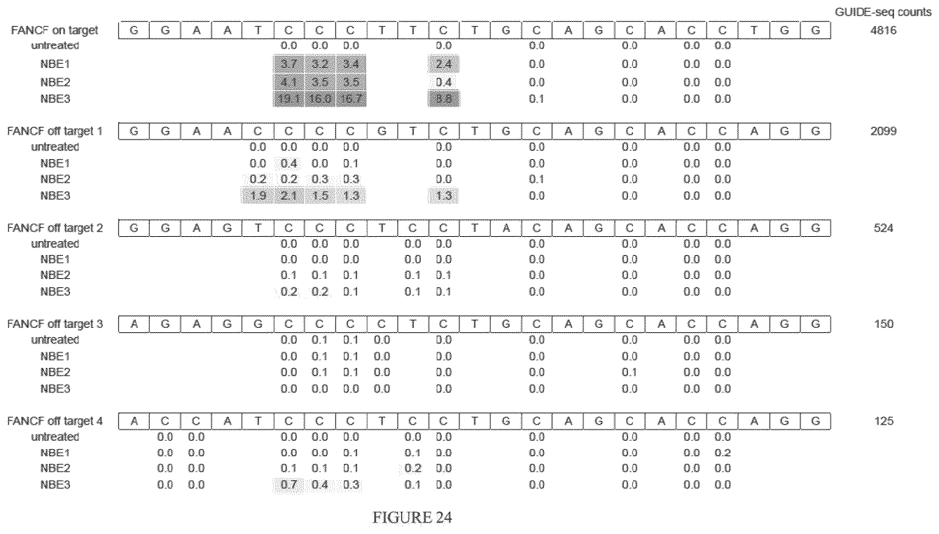

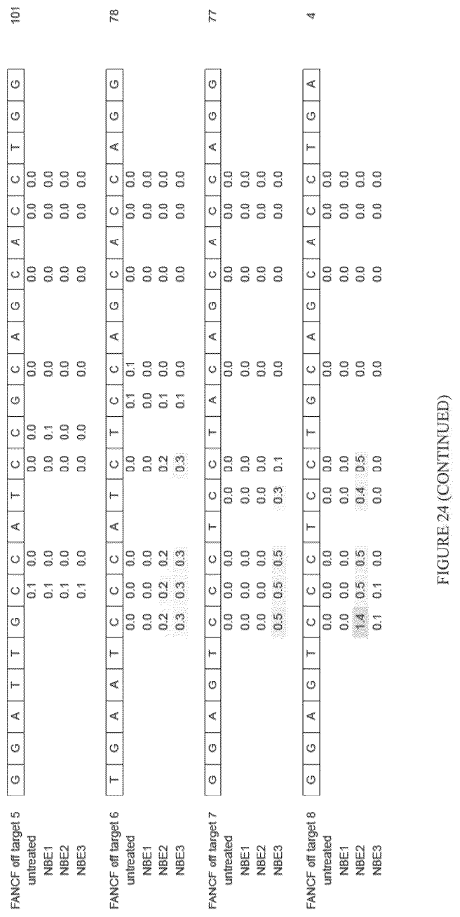

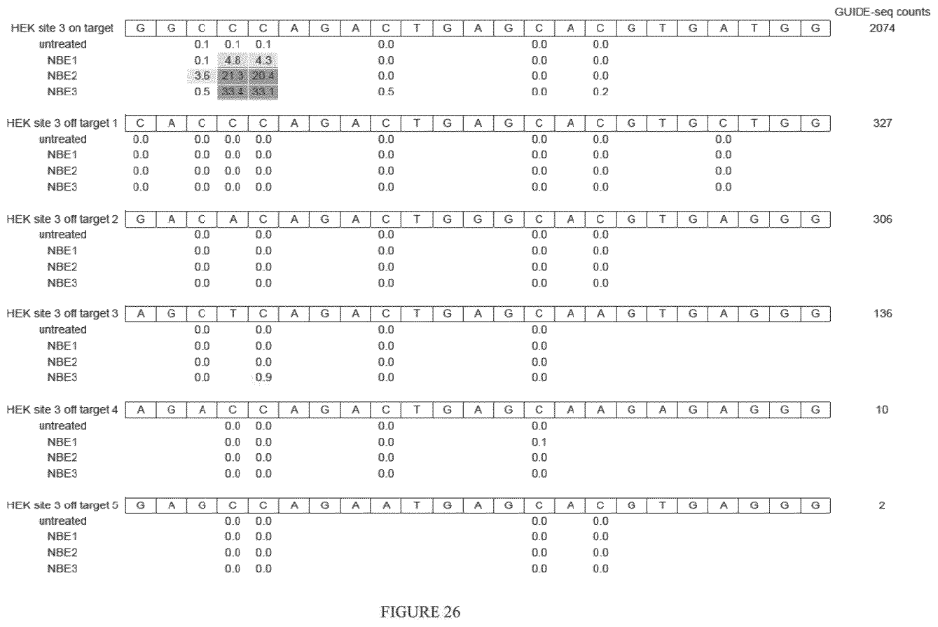

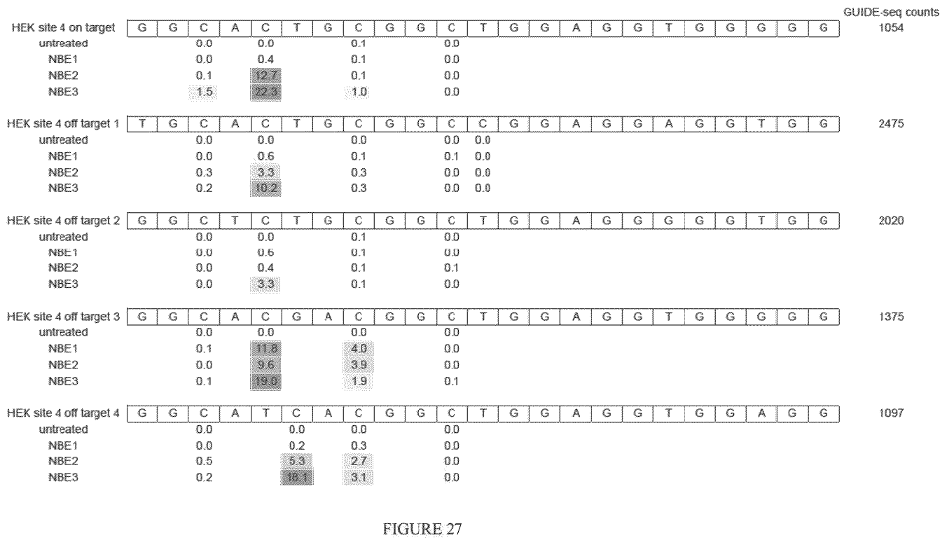

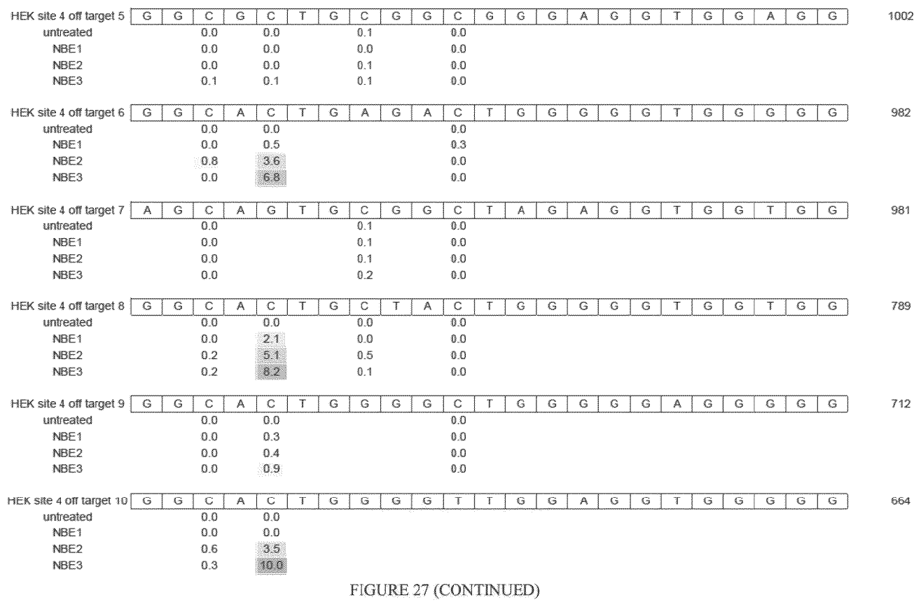

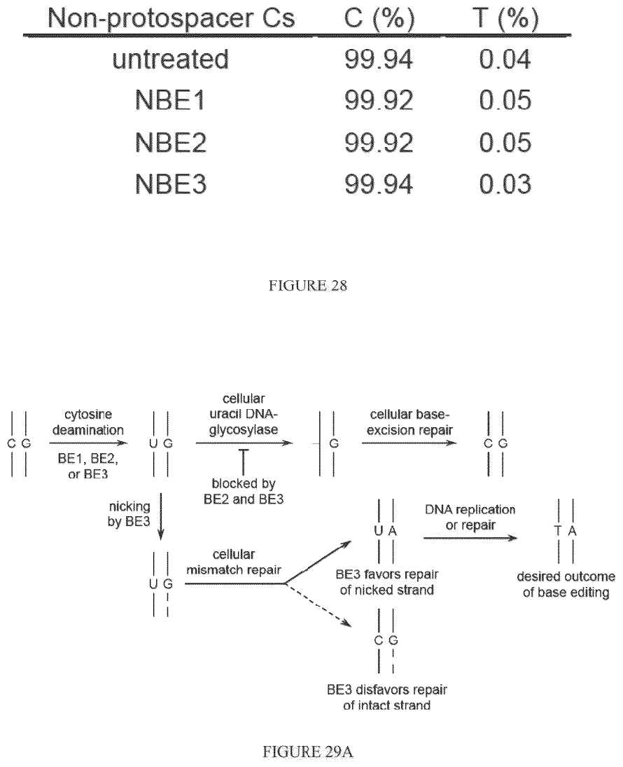

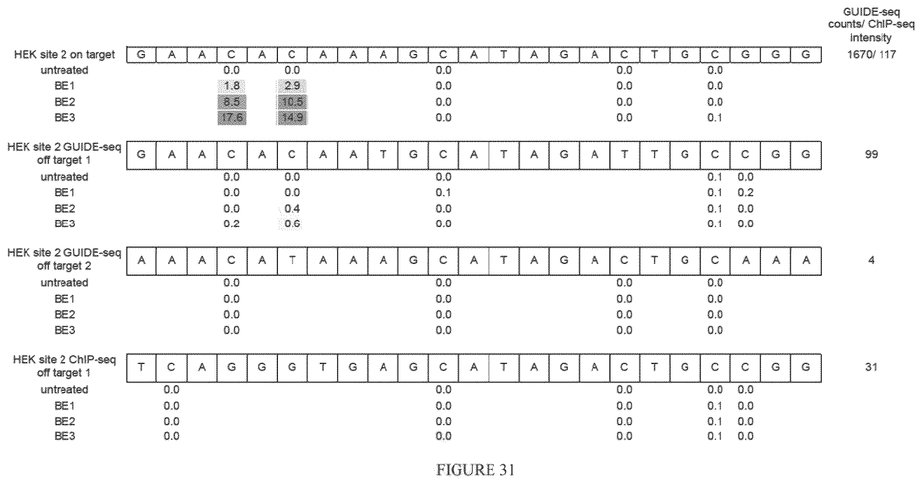

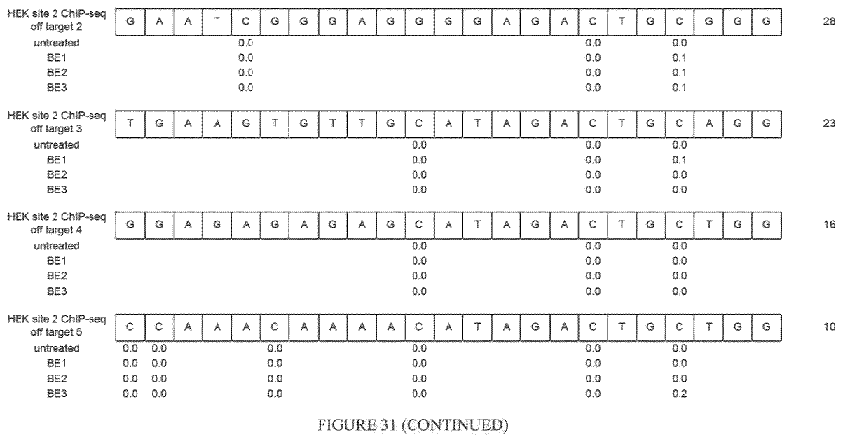

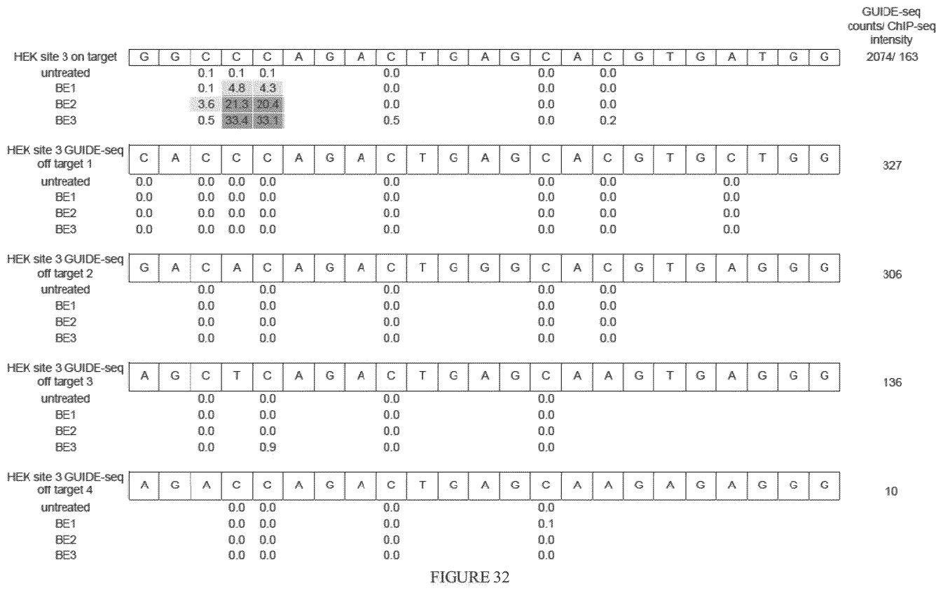

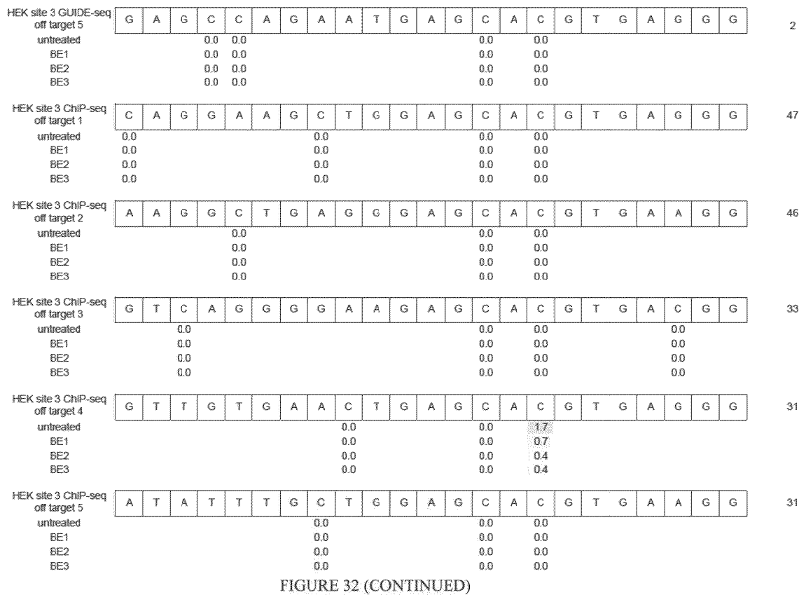

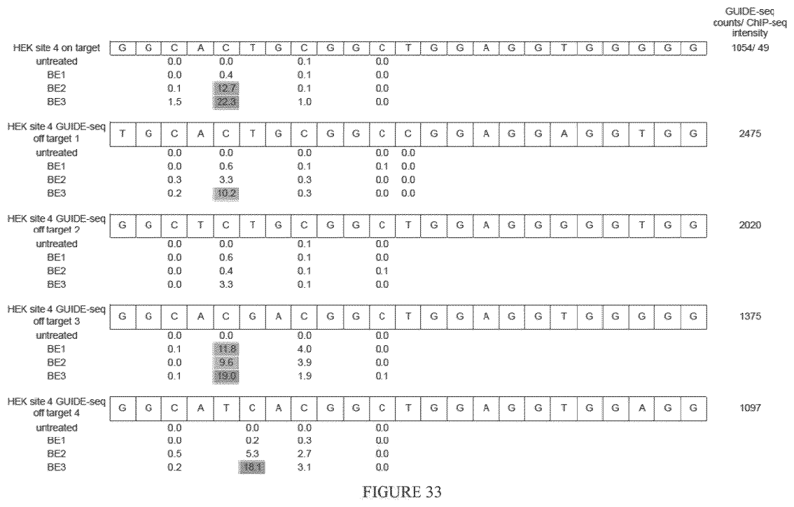

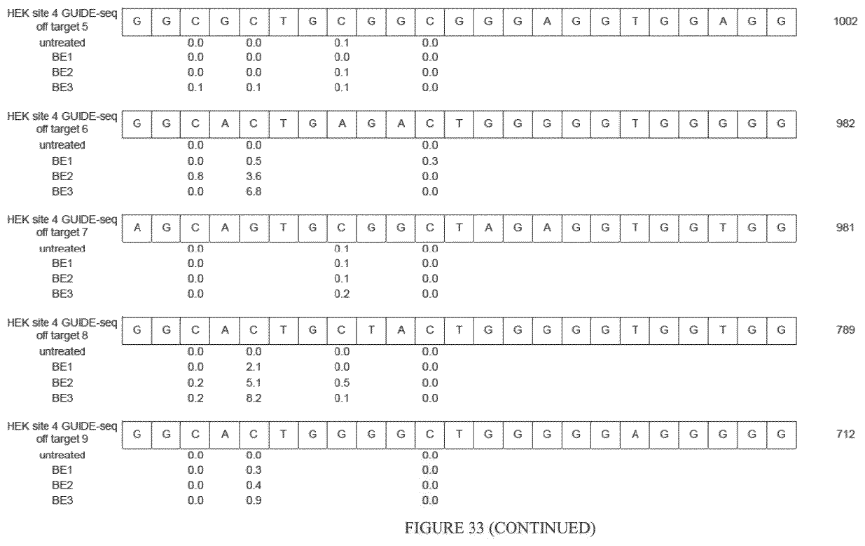

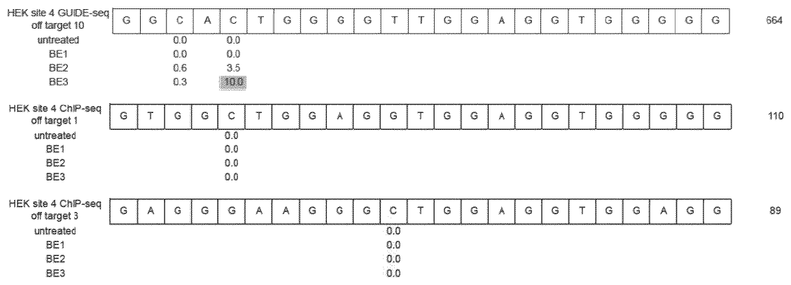

Next, the off-target activity of NBE1, NBE2, and NBE3 in human cells was evaluated. The off-target activities of Cas9, dCas9, and Cas9 nickase have been extensively studied (FIGS. 23 to 24 and 31 to 33).^{54,60-62} Because the sequence preference of rAPOBEC1 has been shown to be independent of DNA bases more than one base from the target C,⁶³ consistent with the sequence context independence observed in FIGS. 12A to 12B, it was assumed that potential off-target activity of nucleobase editors arises from off-target Cas9 binding. Since only a fraction of Cas9 off-target sites will have a C within the active window for nucleobase editing, off-target nucleobase editing sites should be a subset of the off-target sites of canonical Cas9 variants. For each of the six sites studied, the top ten known Cas9 off-target loci in human cells that were previously determined using the GUIDE-seq method were sequenced (FIGS. 23 to 27 and 31 to 33).⁵⁴⁻⁶¹ Detectable off-target nucleobase editing at only a subset (16/34, 47% for NBE1 and NBE2, and 17/34, 50% for NBE3) of known dCas9 off-target loci was observed. In all cases, the off-target base-editing substrates contained a C within the five-base target window. In general, off-target C to T conversion paralleled off-target Cas9 nuclease-mediated genome modification frequencies (FIGS. 23 to 27). Also monitored were C to T conversions at 2,500 distinct cytosines surrounding the six on-target and 34 off-target loci tested, representing a total of 14,700,000 sequence reads derived from approximately 1.8×10⁶ cells, and observed no detectable increase in C to T conversions at any of these other sites upon NBE1, NBE2, or NBE3 treatment compared to that of untreated cells (FIG. 28). Taken together, these findings suggest that off-target substrates of nucleobase editors include a subset of Cas9 off-target substrates, and that nucleobase editors in human cells do not induce untargeted C to T conversion throughout the genome at levels that can be detected by the methods used here. No substantial change was observed in editing efficiency between non-passaged HEK293T cells (editing observed in 1.8% to 2.6% of sequenced strands for the three target Cs with BE2, and 6.2% to 14.3% with BE3) and cells that had undergone approximately five cell divisions after base editing (editing observed in 1.9% to 2.3% of sequenced strands for the same target Cs with BE2, and 6.4% to 14.5% with BE3), confirming that base edits in these cells are durable (Extended Data FIG. 6).

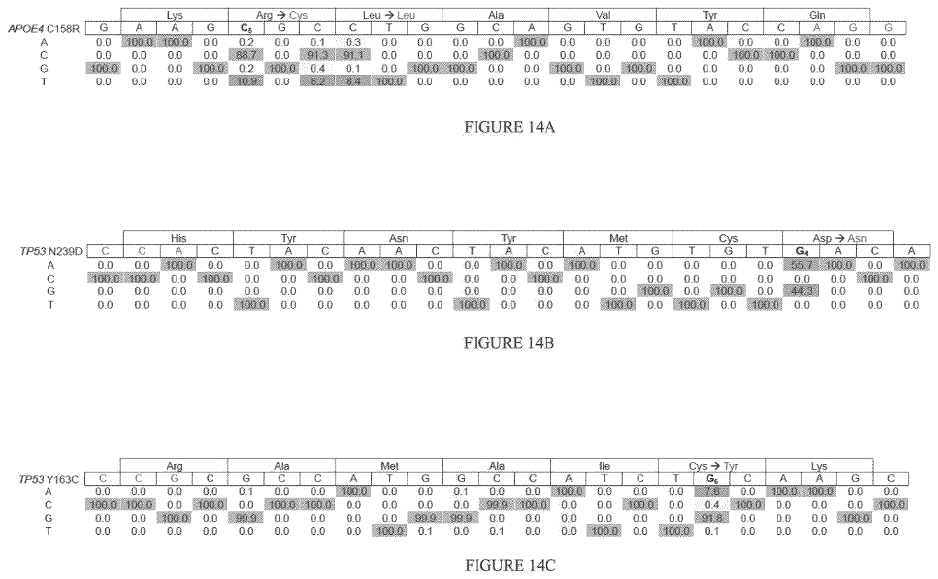

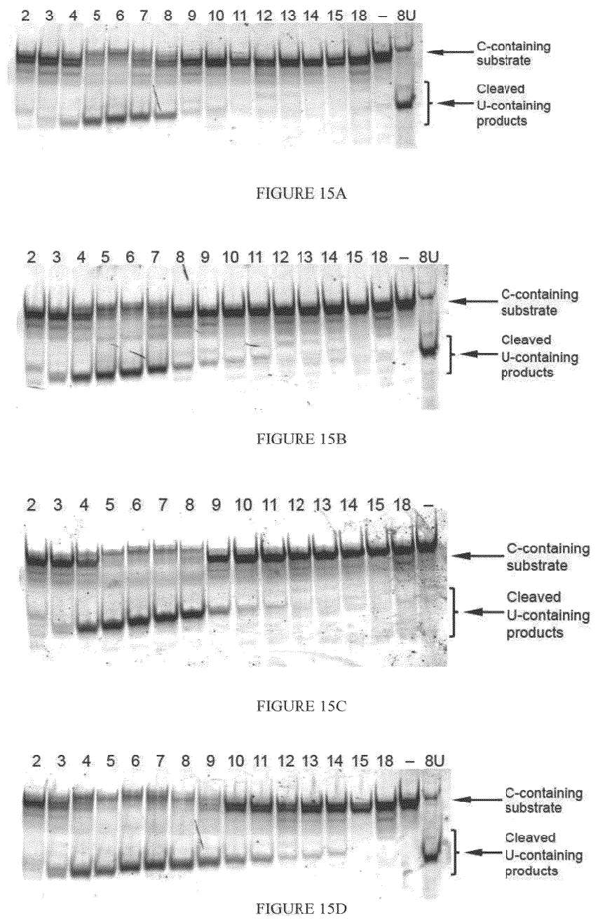

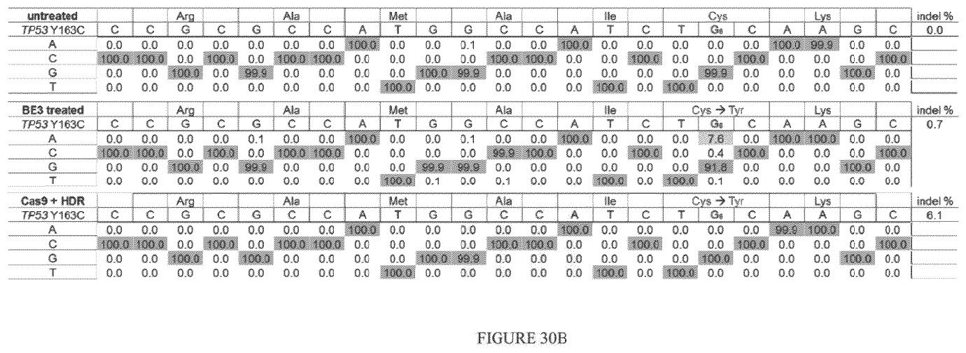

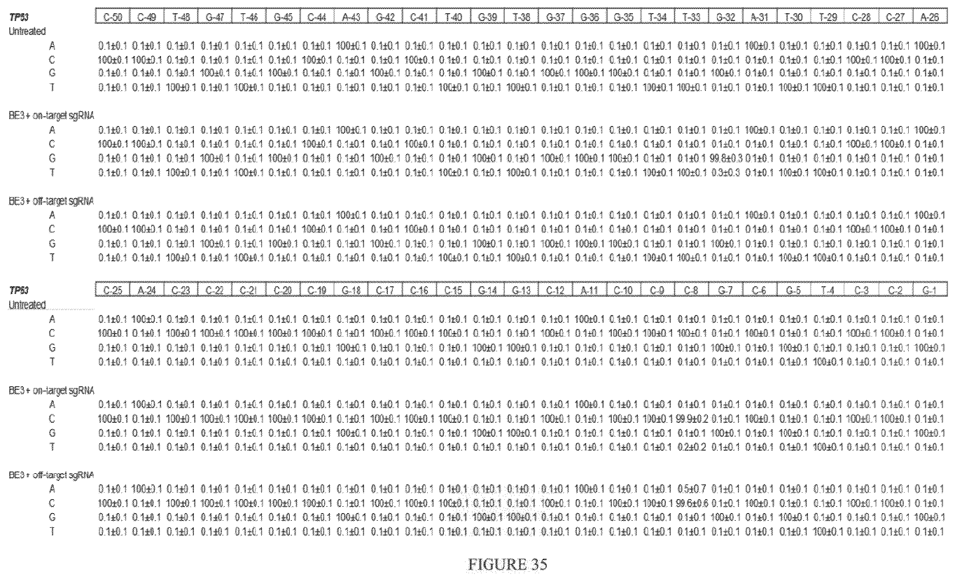

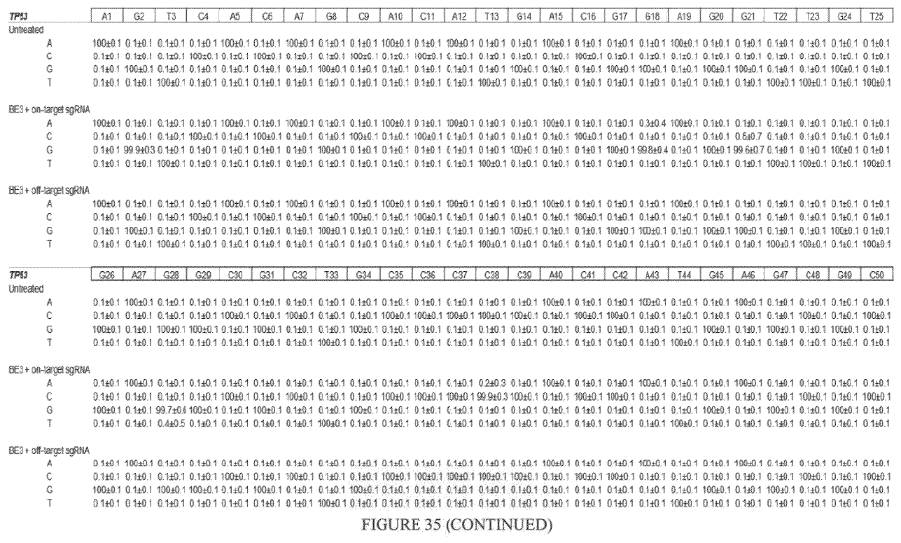

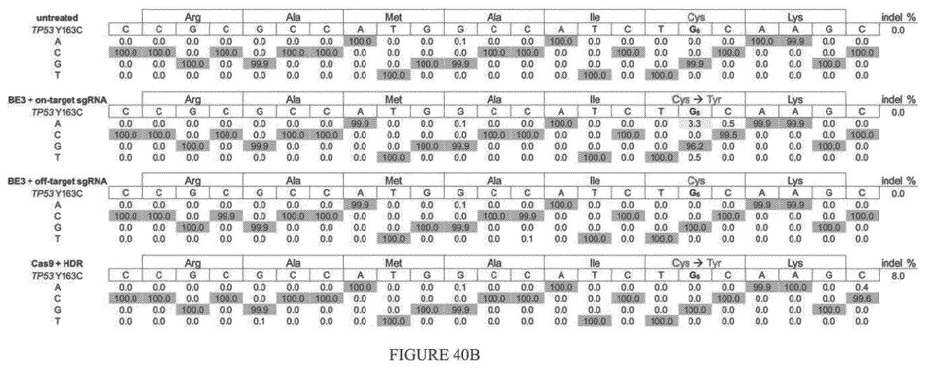

Finally, the potential of nucleobase editing to correct three disease-relevant mutations in mammalian cells was tested. The apolipoprotein E gene variant APOE4 encodes two Arg residues at amino acid positions 112 and 158, and is the largest and most common genetic risk factor for late-onset Alzheimer's disease.⁶⁴ ApoE variants with Cys residues in positions 112 or 158, including APOE2 (Cys112/Cys158), APOE3 (Cys112/Arg158), and APOE3′ (Arg112/Cys158) have been shown⁶⁵ or are presumed⁸¹ to confer substantially lower Alzheimer's disease risk than APOE4. Encouraged by the ability of NBE1 to convert APOE4 to APOE3′ in vitro (FIGS. 16A to 16B), this conversion was attempted in immortalized mouse astrocytes in which the endogenous murine APOE gene has been replaced by human APOE4 (Taconic). DNA encoding NBE3 and an appropriate sgRNA was delivered into these astrocytes by nucleofection (nucleofection efficiency of 25%), extracted genomic DNA from all treated cells two days later, and measured editing efficiency by HTS. Conversion of Arg158 to Cys158 was observed in 58-75% of total DNA sequencing reads (44% of nucleofected astrocytes) (FIGS. 14A to 14C and FIG. 30A). Also observed was 36-50% editing of total DNA at the third position of codon 158 and 38-55% editing of total DNA at the first position of Leu159, as expected since all three of these Cs are within the active nucleobase editing window. However, neither of the other two C→T conversions results in a change in the amino acid sequence of the ApoE3′ protein since both TGC and TGT encode Cys, and both CTG and TTG encode Leu. From >1,500,000 sequencing reads derived from 1×10⁶ cells evidence of 1.7% indels at the targeted locus following NBE3 treatment was observed (FIG. 35). In contrast, identical treatment of astrocytes with wt Cas9 and donor ssDNA resulted in 0.1-0.3% APOE4 correction and 26-40% indels at the targeted locus, efficiencies consistent with previous reports of single-base correction using Cas9 and HDR^{45,75} (FIG. 30A and FIG. 40A). Astrocytes treated identically but with an sgRNA targeting the VEGFA locus displayed no evidence of APOE4 base editing (FIG. 34 and FIG. 40A). These results demonstrate how nucleobase editors can effect precise, single-amino acid changes in the coding sequence of a protein as the major product of editing, even when their processivity results in more than one nucleotide change in genomic DNA. The off-target activities of Cas9, dCas9, and Cas9 nickase have been extensively studied.^{54,60-62} In general, off-target C to T conversions by BE1, BE2, and BE3 paralleled off-target Cas9 nuclease-mediated genome modification frequencies.

The dominant-negative p53 mutations Tyr163Cys and Asn239Asp are strongly associated with several types of cancer.⁶⁶⁻⁶⁷ Both of these mutations can be corrected by a C to T conversion on the template strand (FIGS. 16A to 16B). A human breast cancer cell line homozygous for the p53 Tyr163Cys mutation (HCC1954 cells) was nucleofected with DNA encoding NBE3 and an sgRNA programmed to correct Tyr163Cys. Because the nucleofection efficiency of HCC1954 cells was <10%, a plasmid expressing IRFP was co-nucleofected into these cells to enable isolation of nucleofected cells by fluorescence-activated cell sorting two days after treatment. HTS of genomic DNA revealed correction of the Tyr163Cys mutation in 7.6% of nucleofected HCC1954 cells (FIG. 30B and FIG. 40A to 40B). Also nucleofected was a human lymphoma cell line that is heterozygous for p53 Asn239Asp (ST486 cells) with DNA encoding NBE2 and an sgRNA programmed to correct Asn239Asp with 92% nucleofection efficiency). Correction of the Asn239Asp mutation was observed in 11% of treated ST486 cells (12% of nucleofected ST486 cells). Consistent with the findings in HEK cells, no indels were observed from the treatment of ST486 cells with NBE2, and 0.6% indel formation from the treatment of HCC1954 cells with NBE3. No other DNA changes within at least 50 base pairs of both sides of the protospacer were detected at frequencies above that of untreated controls out of >2,000,000 sequencing reads derived from 2×10⁵ cells (FIGS. 14A to 14C, FIG. 30B and Table 1). These results collectively represent the conversion of three disease-associated alleles in genomic DNA into their wild-type forms with an efficiency and lack of other genome modification events that is, to our knowledge, not currently achievable using other methods.

To illuminate the potential relevance of nucleobase editors to address human genetic diseases, the NCBI ClinVar database⁶⁸ was searched for known genetic diseases that could in principle be corrected by this approach. ClinVar was filtered by first examining only single nucleotide polymorphisms (SNPs), then removing any nonpathogenic variants. Out of the 24,670 pathogenic SNPs, 3,956 are caused by either a T to C, or an A to G, substitution. This list was further filtered to only include variants with a nearby NGG PAM that would position the SNP within the deamination activity window, resulting in 1,089 clinically relevant pathogenic gene variants that could in principle be corrected by the nucleobase editors described here (FIG. 21 and Table 1). To illuminate the potential relevance of base editors to address human genetic diseases, the NCBI ClinVar database⁶⁸ was searched for known genetic diseases that could in principle be corrected by this approach. ClinVar was filtered by first examining only single nucleotide polymorphisms (SNPs), then removing any non-pathogenic variants. Out of the 24,670 pathogenic SNPs, 3,956 are caused by either a T to C, or an A to G, substitution. This list was further filtered to only include variants with a nearby NGG PAM that would position the SNP within the deamination activity window, resulting in 911 clinically relevant pathogenic gene variants that could in principle be corrected by the base editors described here. Of these, 284 contain only one C within the base editing activity window. A detailed list of these pathogenic mutations can be found in Table 1.

TABLE 1
List of 911 base-editable gene variants associated with human disease with an
NGG PAM (SEQ ID NOs: 747 to 1868 appear from top to bottom below, respectively). The “Y”
in the protospacer and PAM sequences indicates the base to be edited, e.g., C.
(SEQ ID NOs: 747 to 1868 appear from top to bottom below, respectively)

| | | Protospacer and | |
|---|---|---|---|
| dbSNP # | Genotype | PAM sequence(s) | Associated genetic disease |
| 755445790 | NM_000391.3(TPP1): | TTTYTTTTTTTTTTTTTTTGAGG | Ceroid lipofuscinosis, |
| | c.887 − 10A > G | | neuronal, 2 |
| 113994167 | NM_000018.3(ACADVL): | TTTGYGGTGGAGAGGGGCTTCGG, | Very long chain acyl-CoA |
| | c.848T > C (p.Val283Ala) | TTGYGGTGGAGAGGGGCTTCGGG | dehydrogenase deficiency |
| 119470018 | NM_024996.5(GFM1): | TTGYTAATAAAAGTTAGAAACGG | Combined oxidative |
| | c.521A > G (p.Asn174Ser) | | phosphorylation deficiency 1 |
| 115650537 | NM_000426.3(LAMA2): | TTGAYAGGGAGCAAGCAGTTCGG, | Merosin deficient congenital |
| | c.8282T > C (p.Ile2761Thr) | TGAYAGGGAGCAAGCAGTTCGGG | muscular dystrophy |
| 587777752 | NM_014946.3(SPAST): | TTCYGTAAAACATAAAAGTCAGG | Spastic paraplegia 4, |
| | c.1688 − 2A > G | | autosomal dominant |
| 794726821 | NM_001165963.1(SCN1A): | TTCYGGTTTGTCTTATATTCTGG | Severe myoclonic epilepsy in |
| | c.4055T > C (p.Leu1352Pro) | | infancy |
| 397514745 | NM_001130089.1(KARS): | CTTCYATGATCTTCGAGGAGAGG, | Deafness, autosomal recessive |
| | c.517T > C (p.Tyr173His) | TTCYATGATCTTCGAGGAGAGGG | 89 |
| 376960358 | NM_001202.3(BMP4): | TTCGTGGYGGAAGCTCCTCACGG | Microphthalmia syndromic 6 |
| | c.362A > G (p.His121Arg) | | |
| 606231280 | NM_001287223.1(SCN11A): | CTTCAYTGTGGTCATTTTCCTGG, | Episodic pain syndrome, |
| | c.1142T > C (p.Ile381Thr) | TTCAYTGTGGTCATTTTCCTGGG | familial, 3 |
| 387906735 | m.608A > G | TTCAGYGTATTGCTTTGAGGAGG | |
| 199474663 | m.3260A > G | TTAAGTTYTATGCGATTACCGGG | Cardiomyopathy with or |
| | | | without skeletal myopathy |
| 104894962 | NM_003413.3(ZIC3): | TGTGTTYGCGCAGGGAGCTCGGG, | Heterotaxy, visceral, X-linked |
| | c.1213A > G (p.Lys405Glu) | ATGTGTTYGCGCAGGGAGCTCGG | |
| 796053181 | NM_021007.2(SCN2A): | TGTGGYGGCCATGGCCTATGAGG | not provided |
| | c.1271T > C (p.Val424Ala) | | |
| 267606788 | NM_000129.3(F13A1): | TGTGAYGGACAGAGCACAAATGG | Factor xiii, a subunit, |
| | c.728T > C (p.Met243Thr) | | deficiency of |
| 397514503 | NM_003863.3(DPM2): | TGTAGYAGGTGAAGATGATCAGG | Congenital disorder of |
| | c.68A > G (p.Tyr23Cys) | | glycosylation type 1u |
| 104893973 | NM_000416.2(IFNGR1): | TGTAATAYTTCTGATCATGTTGG | Disseminated atypical |
| | c.260T > C (p.Ile87Thr) | | mycobacterial infection, |
| | | | Mycobacterium tuberculosis, |
| | | | susceptibility to |
| 121908466 | NM_005682.6(ADGRG1): | TGGYAGAGGCCCCTGGGGTCAGG | Polymicrogyria, bilateral |
| | c.263A > G (p.Tyr88Cys) | | frontoparietal |
| 147952488 | NM_002437.4(MPV17): | TGGYAAGTTCTCCCCTCAACAGG | Navajo neurohepatopathy |
| | c.186 + 2T > C | | |
| 121909537 | NM_001145.4(ANG): | TGGTTYGGCATCATAGTGCTGGG, | Amyotrophic lateral sclerosis |
| | c.121A > G (p.Lys41Glu) | GTGGTTYGGCATCATAGTGCTGG | type 9 |
| 121918489 | NM_000141.4(FGFR2): | TGGGGAAYATACGTGCTTGGCGG, | Crouzon syndrome |
| | c.1018T > C (p.Tyr340His) | GGGGAAYATACGTGCTTGGCGGG | |
| 121434463 | m.12320A > G | GAGTYGCACCAAAATTTTTGGGG, | Mitochondrial myopathy |
| | | GGAGTYGCACCAAAATTTTTGGG, | |
| | | TGGAGTYGCACCAAAATTTTTGG | |
| 121908046 | NM_000403.3(GALE): | TGGAAGYTATCGATGACCACAGG | UDPglucose-4-epimerase |
| | c.101A > G (p.Asn34Ser) | | deficiency |
| 431905512 | NM_003764.3(STX11): | TGCYGGTGGCCGACGTGAAGCGG | Hemophagocytic |
| | c.173T > C (p.Leu58Pro) | | lymphohistiocytosis, familial, |
| | | | 4 |
| 121917905 | NM_000124.3(ERCC6): | TGCYAAAAGACCCAAAACAAAGG | Cerebro-oculo-facio-skeletal |
| | c.2960T > C (p.Leu987Pro) | | syndrome |
| 121918500 | NM_000141.4(FGFR2): | TGCTYGATCCACTGGATGTGGGG, | Crouzon syndrome |
| | c.874A > G (p.Lys292Glu) | GTGCTYGATCCACTGGATGTGGG, | |
| | | CGTGCTYGATCCACTGGATGTGG | |
| 60431989 | NM_000053.3(ATP7B): | TGCTGAYTGGAAACCGTGAGTGG | Wilson disease |
| | c.3443T > C (p.Ile1148Thr) | | |
| 78950939 | NM_000250.1(MPO): | GTGCGGYATTTGTCCTGCTCCGG, | Myeloperoxidase deficiency |
| | c.518A > G (p.Tyr173Cys) | TGCGGYATTTGTCCTGCTCCGGG | |
| 115677373 | NM_201631.3(TGM5): | TGCGGAGYGGACGGGCAGCGTGG | Peeling skin syndrome, acral |
| | c.763T > C (p.Trp255Arg) | | type |
| 5030804 | NM_000551.3(VHL): | GCGAYTGCAGAAGATGACCTGGG, | Von Hippel-Lindau syndrome |
| | c.233A > G (p.Asn78Ser) | TGCGAYTGCAGAAGATGACCTGG | |
| 397508328 | NM_000492.3(CFTR): | GCAYGGTCTCTCGGGCGCTGGGG, | Cystic fibrosis |
| | c.1A > G (p.Met1Val) | TGCAYGGTCTCTCGGGCGCTGGG, | |
| | | CTGCAYGGTCTCTCGGGCGCTGG | |
| 137853299 | NM_000362.4(TIMP3): | TGCAGYAGCCGCCCTTCTGCCGG | Sorsby fundus dystrophy |
| | c.572A > G (p.Tyr191Cys) | | |
| 121908549 | NM_000334.4(SCN4A): | TGAYGGAGGGGATGGCGCCTAGG | |
| | c.3478A > G (p.Ile1160Val) | | |
| 121909337 | NM_001451.2(FOXF1): | TGATGYGAGGCTGCCGCCGCAGG | Alveolar capillary dysplasia |
| | c.1138T > C (p.Ter380Arg) | | with misalignment of |
| | | | pulmonary veins |
| 281875320 | NM_005359.5(SMAD4): | TGAGYATGCATAAGCGACGAAGG | Myhre syndrome |
| | c.1500A > G (p.Ile500Met) | | |
| 730880132 | NM_170707.3(LMNA): | TGAGTYTGAGAGCCGGCTGGCGG | Primary dilated |
| | c.710T > C (p.Phe237Ser) | | cardiomyopathy |
| 281875322 | NM_005359.5(SMAD4): | TGAGTAYGCATAAGCGACGAAGG | Hereditary cancer-predisposing |
| | c.1498A > G (p.Ile500Val) | | syndrome, Myhre syndrome |
| 72556283 | NM_000531.5(OTC): | TGAGGYAATCAGCCAGGATCTGG | not provided |
| | c.527A > G (p.Tyr176Cys) | | |
| 74315311 | NM_020435.3(GJC2): | TGAGAYGGCCCACCTGGGCTTGG, | Leukodystrophy, |
| | c.857T > C (p.Met286Thr) | GAGAYGGCCCACCTGGGCTTGGG | hypomyelinating, 2 |
| 121912495 | NM_170707.3(LMNA): | TCTYGGAGGGCGAGGAGGAGAGG | Congenital muscular |
| | c.1139T > C (p.Leu380Ser) | | dystrophy, LMNA-related |
| 128620184 | NM_000061.2(BTK): | TCTYGATGGCCACGTCGTACTGG | X-linked agammaglobulinemia |
| | c.1288A > G (p.Lys430Glu) | | |
| 118192252 | NM_004519.3(KCNQ3): | TCTTTAYTGTTTAAGCCAACAGG | Benign familial neonatal |
| | c.1403A > G (p.Asn468Ser) | | seizures 2, not specified |
| 121909142 | NM_001300.5(KLF6): | TCTGYGGACCAAAATCATTCTGG | |
| | c.190T > C (p.Trp64Arg) | | |
| 104895503 | NM_001127255.1(NLRP7): | TCTGGYTGATACTCAAGTCCAGG | Hydatidiform mole |
| | c.2738A > G (p.Asn913Ser) | | |
| 587783035 | NM_000038.5(APC): | TCCYAGTAAGAAACAGAATATGG | Familial adenomatous |
| | c.1744 − 2A > G | | polyposis 1 |
| 72556289 | NM_000531.5(OTC): | TCCYAAAAGGCACGGGATGAAGG | not provided |
| | c.541 − 2A > G | | |
| 28937313 | NM_005502.3(ABCA1): | TCCAYTGTGGCCCAGGAAGGAGG, | Tangier disease |
| | c.2804A > G (p.Asn935Ser) | CGCTCCAYTGTGGCCCAGGAAGG | |
| 143246552 | NM_001003811.1(TEX11): | TCCAYGGTCAAGTCAGCCTCAGG, | Spermatogenic failure, X- |
| | c.511A > G (p.Met171Val) | CCAYGGTCAAGTCAGCCTCAGGG | linked, 2 |
| 587776451 | NM_002049.3(GATA1): | CTCCAYGGAGTTCCCTGGCCTGG, | GATA-1-related |
| | c.2T > C (p.Met1Thr) | TCCAYGGAGTTCCCTGGCCTGGG, | thrombocytopenia with |
| | | CCAYGGAGTTCCCTGGCCTGGGG | dyserythropoiesis |
| 121908403 | NM_021102.3(SPINT2): | TCCAYAGATGAAGTTATTGCAGG | Diarrhea 3, secretory sodium, |
| | c.488A > G (p.Tyr163Cys) | | congenital, syndromic |
| 281874738 | NM_000495.4(COL4A5): | CTCCAGYAAGTTATAAAATTTGG, | Alport syndrome, X-linked |
| | c.438 + 2T > C | TCCAGYAAGTTATAAAATTTGGG | recessive |
| 730880279 | NM_030653.3(DDX11): | TCCAGGYGCGGGCGTCATGCTGG, | Warsaw breakage syndrome |
| | c.2271 + 2T > C | CCAGGYGCGGGCGTCATGCTGGG | |
| 28940272 | NM_017890.4(VPS13B): | TCAYTGATAAGCAGGGCCCAGGG, | Cohen syndrome, not specified |
| | c.8978A > G (p.Asn2993Ser) | TTCAYTGATAAGCAGGGCCCAGG | |
| 137852375 | NM_000132.3(F8): | TCAYGGTGAGTTAAGGACAGTGG | Hereditary factor VIII |
| | c.5372T > C (p.Met1791Thr) | | deficiency disease |
| 11567847 | NM_021961.5(TEAD1): | TCATATTYACAGGCTTGTAAAGG | |
| | c.1261T > C (p.Tyr?His) | | |
| 786203989 | NM_016069.9(PAM16): | CATAGTYCTGCAGAGGAGAGGGG, | Chondrodysplasia, |
| | c.226A > G (p.Asn76Asp) | TCATAGTYCTGCAGAGGAGAGGG | megarbane-dagher-melki type |
| 587776437 | NC_012920.1:m.9478T > C | TCAGAAGYTTTTTTCTTCGCAGG | Leigh disease |
| 121912474 | NM_000424.3(KRT5): | TCAAGTGYGTCCTTCCGGAGCGG, | Epidermolysis bullosa |
| | c.20T > C (p.Val7Ala) | CAAGTGYGTCCTTCCGGAGCGGG, | simplex, Koebner type |
| | | AAGTGYGTCCTTCCGGAGCGGGG, | |
| | | AGTGYGTCCTTCCGGAGCGGGGG | |
| 104886461 | NM_020533.2(MCOLN1): | AGGTACYGTGGGCAGAGAAGGGG, | Ganglioside sialidase |
| | c.406 − 2A > G | TACYGTGGGCAGAGAAGGGGAGG, | deficiency |
| | | CAGGTACYGTGGGCAGAGAAGGG | |
| 104894275 | NM_000317.2(PTS): | TAAYTGTGCCCATGGCCATTTGG | 6-pyruvoyl-tetrahydropterin |
| | c.155A > G (p.Asn52Ser) | | synthase deficiency |
| 587777562 | NM_015599.2(PGM3): | TAAATGAYTGAGTTTGCCCTTGG | Immunodeficiency 23 |
| | c.737A > G (p.Asn246Ser) | | |
| 121964906 | NM_000027.3(AGA): | GTTATAYGTGCCAATGTGACTGG | Aspartylglycosaminuria |
| | c.916T > C (p.Cys306Arg) | | |
| 28941769 | NM_000356.3(TCOF1): | GTGTGTAYAGATGTCCAGAAGGG | Treacher collins syndrome 1 |
| | c.149A > G (p.Tyr50Cys) | | |
| 121434464 | m.12297T > C | GTCYTAGGCCCCAAAAATTTTGG | Cardiomyopathy, |
| | | | mitochondrial |
| 121908407 | NM_054027.4(ANKH): | GTCGAGAYGCTGGCCAGCTACGG, | Chondrocalcinosis 2 |
| | c.143T > C (p.Met48Thr) | TCGAGAYGCTGGCCAGCTACGGG | |
| 59151893 | NM_000422.2(KRT17): | GTCAYTGAGGTTCTGCATGGTGG, | Pachyonychia congenita type 2 |
| | c.275A > G (p.Asn92Ser) | GCGGTCAYTGAGGTTCTGCATGG | |
| 121909499 | NM_002427.3(MMP13): | GTCAYGAAAAAGCCAAGATGCGG, | |
| | c.272T > C (p.Met91Thr) | TCAYGAAAAAGCCAAGATGCGGG | |
| 61748478 | NM_000552.3(VWF): | GTCAYAGTTCTGGCACGTTTTGG | von Willebrand disease type |
| | c.2384A > G (p.Tyr795Cys) | | 2N |
| 387906889 | NM_006796.2(AFG3L2): | GTAYAGAGGTATTGTTCTTTTGG | Spastic ataxia 5, autosomal |
| | c.1847A > G (p.Tyr616Cys) | | recessive |
| 118203907 | NM_000130.4(F5): | GTAGYAGGCCCAAGCCCGACAGG | Factor V deficiency |
| | c.5189A > G (p.Tyr1730Cys) | | |
| 118203945 | NM_013319.2(UBIAD1): | GTAAGTGYTGACCAAATTACCGG | Schnyder crystalline corneal |
| | c.305A > G (p.Asn102Ser) | | dystrophy |
| 267607080 | NM_005633.3(SOS1): | GGTYGGGAGGGAAAAGACATTGG | Noonan syndrome 4, |
| | c.1294T > C (p.Trp432Arg) | | Rasopathy |
| 137852953 | NM_012464.4(TLL1): | GGTTAYGGTGCCGTTAAGTTTGG | Atrial septal defect 6 |
| | c.1885A > G (p.Ile629Val) | | |
| 118203949 | NM_013319.2(UBIAD1): | GGTGTTGYTGGAATGGAGAATGG | Schnyder crystalline corneal |
| | c.695A > G (p.Asn232Ser) | | dystrophy |
| 137852952 | NM_012464.4(TLL1): | GGGATTGYTGTTCATGAATTGGG | Atrial septal defect 6 |
| | c.713T > C (p.Val238Ala) | | |
| 41460449 | m.3394T > C | GGCYATATACAACTACGCAAAGG | Leber optic atrophy |
| 80357281 | NM_007294.3(BRCA1): | GGGCYAGAAATCTGTTGCTATGG, | Familial cancer of breast, |
| | c.5291T > C (p.Leu1764Pro) | GGCYAGAAATCTGTTGCTATGGG | Breast-ovarian cancer, familial |
| | | | 1 |
| 5030764 | NM_000174.4(GP9): | GGCTGYTGTTGGCCAGCAGAAGG | Bernard-Soulier syndrome |
| | c.182A > G (p.Asn61Ser) | | type C |
| 72556282 | NM_000531.5(OTC): | GGCTGATYACCTCACGCTCCAGG, | not provided |
| | c.526T > C (p.Tyr176His) | GATYACCTCACGCTCCAGGTTGG | |
| 121913594 | NM_000530.6(MPZ): | GGCATAGYGGAAGATCTATGAGG | Charcot-Marie-Tooth disease |
| | c.242A > G (p.His81Arg) | | type 1B |
| 587777736 | NM_017617.3(NOTCH1): | GGCAAGYGCATCAACACGCTGGG, | Adams-Oliver syndrome 1, |
| | c.1285T > C (p.Cys429Arg) | GGGCAAGYGCATCAACACGCTGG | Adams-Oliver syndrome 5 |
| 63750912 | NM_016835.4(MAPT): | GGATAAYATCAAACACGTCCCGG, | Frontotemporal dementia |
| | c.1839T > C (p.Asn613=) | GATAAYATCAAACACGTCCCGGG | |
| 121918075 | NM_000371.3(TTR): | GGAGYAGGGGCTCAGCAGGGCGG, | Amyloidogenic transthyretin |
| | c.401A > G (p.Tyr134Cys) | ATAGGAGYAGGGGCTCAGCAGGG | amyloidosis |
| 730882063 | NM_004523.3(KIF11): | GGAGGYAATAACTTTGTAAGTGG | Microcephaly with or without |
| | c.2547 + 2T > C | | chorioretinopathy, |
| | | | lymphedema, or mental |
| | | | retardation |
| 397516156 | NM_000257.3(MYH7): | GGAGAYGGCCTCCATGAAGGAGG | Primary familial hypertrophic |
| | c.2546T > C (p.Met849Thr) | | cardiomyopathy, |
| | | | Cardiomyopathy |
| 118204430 | NM_000035.3(ALDOB): | GGAAGYGGCGTGCTGTGCTGAGG | Hereditary fructosuria |
| | c.442T > C (p.Trp148Arg) | | |
| 200198778 | NM_013382.5(POMT2): | GGAAGYAGTGGTGGAAGTAGAGG | Congenital muscular |
| | c.1997A > G (p.Tyr666Cys) | | dystrophy, Congenital |
| | | | muscular dystrophy- |
| | | | dystroglycanopathy with brain |
| | | | and eye anomalies, type A2, |
| | | | Muscular dystrophy, |
| | | | Congenital muscular |
| | | | dystrophy-dystroglycanopathy |
| | | | with mental retardation, type |
| | | | B2 |
| 754896795 | NM_004006.2(DMD): | GCTTTTYTTCAAGCTGCCCAAGG | Duchenne muscular dystrophy, |
| | c.6982A > T (p.Lys2328Ter) | | Becker muscular dystrophy, |
| | | | Dilated cardiomyopathy 3B |
| 148924904 | NM_000546.5(TP53): | GCTTGYAGATGGCCATGGCGCGG | Hereditary cancer-predisposing |
| | c.488A > G (p.Tyr163Cys) | | syndrome |
| 786204770 | NM_016035.4(COQ4): | GCTGTYGGCCGCCGGCTCCGCGG | COENZYME Q10 |
| | c.155T > C (p.Leu52Ser) | | DEFICIENCY, PRIMARY, 7 |
| 121909520 | NM_001100.3(ACTA1): | CGGYTGGCCTTGGGATTGAGGGG, | Nemaline myopathy 3 |
| | c.350A > G (p.Asn117Ser) | GCGGYTGGCCTTGGGATTGAGGG, | |
| | | CGCGGYTGGCCTTGGGATTGAGG | |
| 587776879 | NM_004656.3(BAP1): | GCCYGGGGAAAAACAGAGTCAGG | Tumor predisposition |
| | c.438 − 2A > G | | syndrome |
| 727504434 | NM_000501.3(ELN): | GCCYGAAAACACAGCCACAGAGG | Supravalvar aortic stenosis |
| | c.890 − 2A > G | | |
| 119455953 | NM_000391.3(TPP1): | GCCGGGYGTTGGTCTGTCTCTGG | Ceroid lipofuscinosis, |
| | c.1093T > C (p.Cys365Arg) | | neuronal, 2 |
| 121964983 | NM_000481.3(AMT): | GCCAGGYGGAAGTCATAGAGCGG | Non-ketotic hyperglycinemia |
| | c.125A > G (p.His42Arg) | | |
| 121908300 | NM_001005741.2(GBA): | GCCAGAYACTTTGTGAAGTAAGG, | Gaucher disease, type 1 |
| | c.751T > C (p.Tyr251His) | CCAGAYACTTTGTGAAGTAAGGG | |
| 786205083 | NM_003494.3(DYSF): | GCCAGAGYGAGTGGCTGGAGTGG | Limb-girdle muscular |
| | c.3443 − 33A > G | | dystrophy, type 2B |
| 121908133 | NM_175073.2(APTX): | GCCAAYGGTAACGGGCCTTTGGG, | Adult onset ataxia with |
| | c.602A > G (p.His201Arg) | AGCCAAYGGTAACGGGCCTTTGG | oculomotor apraxia |
| 587777195 | NM_005017.3(PCYT1A): | GCATGYTTGCTCCAACACAGAGG | Spondylometaphyseal |
| | c.571T > C (p.Phe191Leu) | | dysplasia with cone-rod |
| | | | dystrophy |
| 431905520 | NM_014714.3(IFT140): | CAAGCAGYGTGAGCTGCTCCTGG, | Renal dysplasia, retinal |
| | c.4078T > C (p.Cys1360Arg) | GCAGYGTGAGCTGCTCCTGGAGG | pigmentary dystrophy, |
| | | | cerebellar ataxia and skeletal |
| | | | dysplasia |
| 121912889 | NM_001844.4(COL2A1): | GCAGTGGYAGGTGATGTTCTGGG | Spondyloperipheral dysplasia, |
| | c.4172A > G (p.Tyr1391Cys) | | Platyspondylic lethal skeletal |
| | | | dysplasia Torrance type |
| 137854492 | NM_001363.4(DKC1): | GCAGGYAGAGATGACCGCTGTGG | Dyskeratosis congenita X- |
| | c.1069A > G (p.Thr357Ala) | | linked |
| 121434362 | NM_152783.4(D2HGDH): | GCAGGTYACCATCTCCTGGAGGG, | D-2-hydroxyglutaric aciduria 1 |
| | c.1315A > G (p.Asn439Asp) | TGCAGGTYACCATCTCCTGGAGG | |
| 80338732 | NM_002764.3(PRPS1): | GCAAATAYGCTATCTGTAGCAGG | Charcot-Marie-Tooth disease, |
| | c.344T > C (p.Met115Thr) | | X-linked recessive, type 5 |
| 387906675 | NM_000313.3(PROS1): | GATTAYATCTGTAGCCTTCGGGG, | Thrombophilia due to protein |
| | c.701A > G (p.Tyr234Cys) | AGATTAYATCTGTAGCCTTCGGG, | S deficiency, autosomal |
| | | GAGATTAYATCTGTAGCCTTCGG | recessive |
| 28935478 | NM_000061.2(BTK): | GATGGYAGTTAATGAGCTCAGGG, | |
| | c.1082A > G (p.Tyr361Cys) | TGATGGYAGTTAATGAGCTCAGG | |
| 201777056 | NM_005050.3(ABCD4): | GATGAGGYAGATGCACACAAAGG | METHYLMALONIC |
| | c.956A > G (p.Tyr319Cys) | | ACIDURIA AND |
| | | | HOMOCYSTINURIA, cblJ |
| | | | TYPE |
| 121918528 | NM_000098.2(CPT2): | GATAGGYACATATCAAACCAGGG, | Carnitine palmitoyltransferase |
| | c.359A > G (p.Tyr120Cys) | AGATAGGYACATATCAAACCAGG | II deficiency, infantile |
| 267607014 | NM_002942.4(ROBO2): | GAGAYTGGAAATTTTGGCCGTGG | Vesicoureteral reflux 2 |
| | c.2834T > C (p.Ile945Thr) | | |
| 281865192 | NM_025114.3(CEP290): | GATAYTCACAATTACAACTGGGG, | Leber congenital amaurosis 10 |
| | c.2991 + 1655A > G | AGATAYTCACAATTACAACTGGG, | |
| | | GAGATAYTCACAATTACAACTGG | |
| 386833492 | NM_000112.3(SLC26A2): | GAGAGGYGAGAAGAGGGAAGCGG | Diastrophic dysplasia |
| | c.−26 + 2T > C | | |
| 587779773 | NM_001101.3(ACTB): | GAGAAGAYGACCCAGGTGAGTGG | Baraitser-Winter syndrome 1 |
| | c.356T > C (p.Met119Thr) | | |
| 121913512 | NM_000222.2(KIT): | GACTTYGAGTTCAGACATGAGGG, | |
| | c.1924A > G (p.Lys642Glu) | GGACTTYGAGTTCAGACATGAGG | |
| 28939072 | NM_006329.3(FBLN5): | GACAYTGATGAATGTCGCTATGG | Age-related macular |
| | c.506T > C (p.Ile169Thr) | | degeneration 3 |
| 104894248 | NM_000525.3(KCNJ11): | GACAYGGTAGATGATCAGCGGGG, | Islet cell hyperplasia |
| | c.776A > G (p.His259Arg) | TGACAYGGTAGATGATCAGCGGG, | |
| | | ATGACAYGGTAGATGATCAGCGG | |
| 387907132 | NM_016464.4(TMEM138): | GACAYGAAGGGAGATGCTGAGGG, | Joubert syndrome 16 |
| | c.287A > G (p.His96Arg) | AGACAYGAAGGGAGATGCTGAGG | |
| 121918170 | NM_000275.2(OCA2): | GACATYTGGAGGGTCCCCGATGG | Tyrosinase-positive |
| | c.1465A>G (p.Asn489Asp) | | oculocutaneous albinism |
| 122467173 | NM_014009.3(FOXP3): | GACAGAGYTCCTCCACAACATGG | Insulin-dependent diabetes |
| | c.970T > C (p.Phe324Leu) | | mellitus secretory diarrhea |
| | | | syndrome |
| 137852268 | NM_000133.3(F9): | GAAYATATACCAAGGTATCCCGG | Hereditary factor IX deficiency |
| | c.1328T > C (p.Ile443Thr) | | disease |
| 149054177 | NM_001999.3(FBN2): | GAATGTAYGATAATGAACGGAGG | not specified, Macular |
| | c.3740T > C (p.Met1247Thr) | | degeneration, early-onset |
| 137854488 | NM_212482.1(FN1): | GAAGTAAYAGGTGACCCCAGGGG | Glomerulopathy with |
| | c.2918A > G (p.Tyr973Cys) | | fibronectin deposits 2 |
| 786204027 | NM_005957.4(MTHFR): | GAAGGYGTGGTAGGGAGGCACGG, | Homocysteinemia due to |
| | c.1530 + 2T > C | AAGGYGTGGTAGGGAGGCACGGG, | MTHFR deficiency |
| | | AGGYGTGGTAGGGAGGCACGGGG | |
| 104894223 | NM_012193.3(FZD4): | GAAATAYGATGGGGCGCTCAGGG, | Retinopathy of prematurity |
| | c.766A > G (p.Ile256Val) | AGAAATAYGATGGGGCGCTCAGG | |
| 137854474 | NM_000138.4(FBN1): | CTTGYGTTATGATGGATTCATGG | Marfan syndrome |
| | c.3793T > C (p.Cys1265Arg) | | |
| 587784418 | NM_006306.3(SMC1A): | CTTAYAGATCTCATCAATGTTGG | Congenital muscular |
| | c.3254A > G (p.Tyr1085Cys) | | hypertrophy-cerebral |
| | | | syndrome |
| 81002805 | NM_000059.3(BRCA2): | CTTAGGYAAGTAATGCAATATGG | Familial cancer of breast, |
| | c.316 + 2T > C | | Breast-ovarian cancer, familial |
| | | | 2, Hereditary cancer- |
| | | | predisposing syndrome |
| 121909653 | NM_182925.4(FLT4): | CTGYGGATGCACTGGGGTGCGGG, | |
| | c.3104A > G (p.His1035Arg) | TCTGYGGATGCACTGGGGTGCGG | |
| 786205107 | NM_031226.2(CYP19A1): | CTGTGYAAGTAATACAACTTTGG | Aromatase deficiency |
| | c.743 + 2T > C | | |
| 587777037 | NM_001283009.1(RTEL1): | CTGTGTGYGCCAGGGCTGTGGGG | Dyskeratosis congenita, |
| | c.3730T > C (p.Cys1244Arg) | | autosomal recessive, 5 |
| 794728380 | NM_000238.3(KCNH2): | CTGTGAGYGTGCCCAGGGGCGGG, | Cardiac arrhythmia |
| | c.1945 + 6T > C | TGAGYGTGCCCAGGGGGGGGCGG | |
| 267607987 | NM_000251.2(MSH2): | CTGGYAAAAAACCTGGTTTTTGG, | Hereditary Nonpolyposis |
| | c.2005 + 2T > C | TGGYAAAAAACCTGGTTTTTGGG | Colorectal Neoplasms |
| 397509397 | NM_006876.2(B4GAT1): | TGATYTTCAGCCTCCTTTTGGGG, | Congenital muscular |
| | c.1168A > G (p.Asn390Asp) | CTGATYTTCAGCCTCCTTTTGGG, | dystrophy-dystroglycanopathy |
| | | GCTGATYTTCAGCCTCCTTTTGG | with brain and eye anomalies, |
| | | | type A13 |
| 121918381 | NM_000040.1(APOC3): | CTGAAGYTGGTCTGACCTCAGGG, | |
| | c.280A > G (p.Thr94Ala) | GCTGAAGYTGGTCTGACCTCAGG | |
| 104894919 | NM_001015877.1(PHF6): | CTCYTGATGTTGTTGTGAGCTGG | Borjeson-Forssman-Lehmann |
| | c.769A > G (p.Arg257Gly) | | syndrome |
| 267606869 | NM_005144.4(HR): | CTCYAGGGCCGCAGGTTGGAGGG, | Marie Unna hereditary |
| | c.−218A > G | GCTCYAGGGCCGCAGGTTGGAGG, | hypotrichosis 1 |
| | | GGCGCTCYAGGGCCGCAGGTTGG | |
| 139732572 | NM_000146.3(FTL): | CTCAYGGTTGGTTGGCAAGAAGG | L-ferritin deficiency |
| | c.1A > G (p.Met1Val) | | |
| 397515418 | NM_018486.2(HDAC8): | CTCAYGATCTGGGATCTCAGAGG | Cornelia de Lange syndrome 5 |
| | c.1001A > G (p.His334Arg) | | |
| 372395294 | NM_198056.2(SCN5A): | CTCAYAGGCCATTGCGACCACGG | not provided |
| | c.1247A > G (p.Tyr416Cys) | | |
| 104895304 | NM_000431.3(MVK): | CTCAAYAGATGCCATCTCCCTGG | Hyperimmunoglobulin D with |
| | c.803T > C (p.Ile268Thr) | | periodic fever, Mevalonic |
| | | | aciduria |
| 587777188 | NM_001165899.1(PDE4D): | CTATAYTGTTCATCCCCTCTGGG, | Acrodysostosis 2, with or |
| | c.1850T > C (p.Ile617Thr) | ACTATAYTGTTCATCCCCTCTGG | without hormone resistance |
| 398123026 | NM_003867.3(FGF17): | CGTGGYTGGGGAAGGGCAGCTGG | Hypogonadotropic |
| | c.560A > G (p.Asn187Ser) | | hypogonadism 20 with or |
| | | | without anosmia |
| 121964924 | NM_001385.2(DPYS): | CGTAATAYGGGAAAAAGGCGTGG, | Dihydropyrimidinase |
| | c.1078T > C (p.Trp360Arg) | AATAYGGGAAAAAGGCGTGGTGG, | deficiency |
| | | ATAYGGGAAAAAGGCGTGGTGGG | |
| 587777301 | NM_199189.2(MATR3): | CGGYTGAACTCTCAGTCTTCTGG | Myopathy, distal, 2 |
| | c.1864A > G (p.Thr622Ala) | | |
| 200238879 | NM_000527.4(LDLR): | ACTGCGGYATGGGCGGGGCCAGG, | Familial hypercholesterolemia |
| | c.694 + 2T > C | CTGCGGYATGGGCGGGGCCAGGG, | |
| | | CGGYATGGGCGGGGCCAGGGTGG | |
| 142951029 | NM_145046.4(CALR3): | CGGTYTGAAGCGTGCAGAGATGG | Arrhythmogenic right |
| | c.245A > G (p.Lys82Arg) | | ventricular cardiomyopathy, |
| | | | Familial hypertrophic |
| | | | cardiomyopathy 19, |
| | | | Hypertrophic cardiomyopathy |
| 786200953 | NM_006785.3(MALT1): | CGCYTTGAAAAAAAAAGAAAGGG, | Combined immunodeficiency |
| | c.1019 − 2A > G | TCGCYTTGAAAAAAAAAGAAAGG | |
| 120074192 | NM_000218.2(KCNQ1): | CGCYGAAGATGAGGCAGACCAGG | Atrial fibrillation, familial, 3, |
| | c.418A > G (p.Ser140Gly) | | Atrial fibrillation |
| 267606887 | NM_005957.4(MTHFR): | CGCGGYTGAGGGTGTAGAAGTGG | Homocystinuria due to |
| | c.971A > G (p.Asn324Ser) | | MTHFR deficiency |
| 118192117 | NM_000540.2(RYR1): | CGCAYGATCCACAGCACCAATGG | Congenital myopathy with |
| | c.1205T > C (p.Met402Thr) | | fiber type disproportion, |
| | | | Central core disease |
| 199473625 | NM_198056.2(SCN5A): | CGAYGTTGAAGAGGGCAGGCAGG, | Brugada syndrome |
| | c.4978A > G (p.Ile1660Val) | AGCCCGAYGTTGAAGAGGGCAGG | |
| 794726865 | NM_000921.4(PDE3A): | CGAGGYGGTGGTGGTCCAAGTGG | Brachydactyly with |
| | c.1333A > G (p.Thr445Ala) | | hypertension |
| 606231254 | NM_005740.2(DNAL4): | CGAGGYATTGCCAGCAGTGCAGG | Mirror movements 3 |
| | c.153 + 2T > C | | |
| 786204826 | NM_004771.3(MMP20): | CGAAAYGTGTATCTCCTCCCAGG | Amelogenesis imperfecta, |
| | c.611A > G (p.His204Arg) | | hypomaturation type, IIA2 |
| 796053139 | NM_021007.2(SCN2A): | CGAAATGYAAGTCTAGTTAGAGG, | not provided |
| | c.4308 + 2T > C | GAAATGYAAGTCTAGTTAGAGGG | |
| 137854494 | NM_005502.3(ABCA1): | CCTGTGYGTCCCCCAGGGGCAGG, | Tangier disease |
| | c.4429T > C (p.Cys1477Arg) | CTGTGYGTCCCCCAGGGGCAGGG, | |
| | | TGTGYGTCCCCCAGGGGCAGGGG, | |
| | | GTGYGTCCCCCAGGGGCAGGGGG | |
| 786205144 | NM_001103.3(ACTN2): | CCTAAAAYGTTGGATGCTGAAGG | Dilated cardiomyopathy 1AA |
| | c.683T > C (p.Met228Thr) | | |
| 199919568 | NM_007254.3(PNKP): | CCGGYGAGGCCCTGGGGCGGGGG, | not provided |
| | c.1029 + 2T > C | TCCGGYGAGGCCCTGGGGCGGGG, | |
| | | ATCCGGYGAGGCCCTGGGGGGGG, | |
| | | GATCCGGYGAGGCCCTGGGGCGG | |
| 28939079 | NM_018965.3(TREM2): | TGAYCCAGGGGGTCTATGGGAGG, | Polycystic lipomembranous |
| | c.401A > G (p.Asp134Gly) | CGGTGAYCCAGGGGGTCTATGGG, | osteodysplasia with sclerosing |
| | | CCGGTGAYCCAGGGGGTCTATGG | leukoencephalopathy |
| 193302855 | NM_032520.4(GNPTG): | CCCYGAAGGTGGAGGATGCAGGG, | Mucolipidosis III Gamma |
| | c.610 − 2A > G | GCCCYGAAGGTGGAGGATGCAGG | |
| 111033708 | NM_000155.3(GALT): | CCCTYGGGTGCAGGTTTGTGAGG | Deficiency of UDPglucose- |
| | c.499T > C (p.Trp167Arg) | | hexose-1-phosphate |
| | | | uridylyltransferase |
| 28933378 | NM_000174.4(GP9): | CCCAYGTACCTGCCGCGCCCTGG | Bernard Soulier syndrome, |
| | c.70T > C (p.Cys24Arg) | | Bernard-Soulier syndrome |
| | | | type C |
| 364897 | NM_000157.3(GBA): | CCAYTGGTCTTGAGCCAAGTGGG, | Gaucher disease, Subacute |
| | c.680A > G (p.Asn227Ser) | TCCAYTGGTCTTGAGCCAAGTGG | neuronopathic Gaucher |
| | | | disease, Gaucher disease, type |
| | | | 1 |
| 796052551 | NM_000833.4(GRIN2A): | CCAYGTTGTCAATGTCCAGCTGG | not provided |
| | c.2449A > G (p.Met817Val) | | |
| 63751006 | NM_002087.3(GRN): | CCAYGTGGACCCTGGTGAGCTGG | Frontotemporal dementia, |
| | c.2T > C (p.Met1Thr) | | ubiquitin-positive |
| 786203997 | NM_001031.4(RPS28): | TGTCCAYGATGGCGGCGCGGCGG, | Diamond-Blackfan anemia |
| | c.1A > G (p.Met1 Val) | CCAYGATGGCGGCGCGGCGGCGG | with microtia and cleft palate |
| 121908595 | NM_002755.3(MAP2K1): | CCAYAGAAGCCCACGATGTACGG | Cardiofaciocutaneous |
| | c.389A > G (p.Tyr130Cys) | | syndrome 3, Rasopathy |
| 398122910 | NM_000431.3(MVK): | CCAGGYATCCCGGGGGTAGGTGG, | Porokeratosis, disseminated |
| | c.1039 + 2T > C | CAGGYATCCCGGGGGTAGGTGGG | superficial actinic 1 |
| 119474039 | NM_020365.4(EIF2B3): | CCAGAYTGTCAGCAAACACCTGG | Leukoencephalopathy with |
| | c.1037T > C (p.Ile346Thr) | | vanishing white matter |
| 587777866 | NM_000076.2(CDKN1C): | CCAAGYGAGTACAGCGCACCTGG, | Beckwith-Wiedemann |
| | c.*5 + 2T > C | CAAGYGAGTACAGCGCACCTGGG, | syndrome |
| | | AAGYGAGTACAGCGCACCTGGGG | |
| 121918530 | NM_005587.2(MEF2A): | AGAYTACCACCACCTGGTGGAGG, | |
| | c.788A > G (p.Asn263Ser) | CCAAGAYTACCACCACCTGGTGG | |
| 483352818 | NM_000211.4(ITGB2): | CATGYGAGTGCAGGCGGAGCAGG | Leukocyte adhesion deficiency |
| | c.1877 + 2T > C | | type 1 |
| 460184 | NM_000186.3(CFH): | CAGYTGAATTTGTGTGTAAACGG | Atypical hemolytic-uremic |
| | c.3590T > C (p.Val1197Ala) | | syndrome 1 |
| 121908423 | NM_004795.3(KL): | CAGYGGTACAGGGTGACCACGGG, | |
| | c.578A > G (p.His193Arg) | CCAGYGGTACAGGGTGACCACGG | |
| 281860300 | NM_005247.2(FGF3): | GCAGYAGAGCTTGCGGCGCCGGG, | Deafness with labyrinthine |
| | c.146A > G (p.Tyr49Cys) | CAGYAGAGCTTGCGGCGCCGGGG, | aplasia microtia and |
| | | CGCAGYAGAGCTTGCGGCGCCGG | microdontia (LAMM) |
| 28935488 | NM_000169.2(GLA): | CAGTTAGYGATTGGCAACTTTGG | Fabry disease |
| | c.806T > C (p.Val269Ala) | | |
| 587776514 | NM_173560.3(RFX6): | CAGTGGYGAGACTCGCCCGCAGG, | Mitchell-Riley syndrome |
| | c.380 + 2T > C | AGTGGYGAGACTCGCCCGCAGGG | |
| 104894117 | NM_178138.4(LHX3): | CAGGTGGYACACGAAGTCCTGGG | Pituitary hormone deficiency, |
| | c.332A > G (p.Tyr111Cys) | | combined 3 |
| 34878913 | NM_000184.2(HBG2): | CAGAGGTYCTTTGACAGCTTTGG | Cyanosis, transient neonatal |
| | c.125T > C (p.Phe42Ser) | | |
| 120074124 | NM_000543.4(SMPD1): | AGCACYTGTGAGGAAGTTCCTGG, | Sphingomyelin/cholesterol |
| | c.911T > C (p.Leu304Pro) | GCACYTGTGAGGAAGTTCCTGGG, | lipidosis, Niemann-Pick |
| | | CACYTGTGAGGAAGTTCCTGGGG | disease, type A, Niemann-Pick |
| | | | disease, type B |
| 281860272 | NM_005211.3(CSF1R): | CACYGAGGGAAAGCACTGCAGGG, | Hereditary diffuse |
| | c.2320 − 2A > G | GCACYGAGGGAAAGCACTGCAGG | leukoencephalopathy with |
| | | | spheroids |
| 128624216 | NM_000033.3(ABCD1): | CACTGYTGACGAAGGTAGCAGGG, | Adrenoleukodystrophy |
| | c.443A > G (p.Asn148Ser) | GCACTGYTGACGAAGGTAGCAGG | |
| 398124257 | NM_012463.3(ATP6V0A2): | CACTGYGAGTAAGCTGGAAGTGG | Cutis laxa with osteodystrophy |
| | c.825 + 2T > C | | |
| 267606679 | NM_004183.3(BEST1): | CACTGGYGTATACACAGGTGAGG | Vitreoretinochoroidopathy |
| | c.704T > C (p.Val235Ala) | | dominant |
| 397514518 | NM_000344.3(SMN1): | CACTGGAYATGGAAATAGAGAGG | Kugelberg-Welander disease |
| | c.388T > C (p.Tyr130His) | | |
| 143946794 | NM_001946.3(DUSP6): | CACTAYTGGGGTCTCGGTCAAGG | Hypogonadotropic |
| | c.566A > G (p.Asn189Ser) | | hypogonadism 19 with or |
| | | | without anosmia |
| 397516076 | NM_000256.3(MYBPC3): | GCACGYGAGTGGCCATCCTCAGG, | Familial hypertrophic |
| | c.821 + 2T > C | CACGYGAGTGGCCATCCTCAGGG | cardiomyopathy 4, not |
| | | | specified |
| 149977726 | NM_001257988.1(TYMP): | CACGAGTYTCTTACTGAGAATGG, | |
| | c.665A > G (p.Lys222Arg) | GAGTYTCTTACTGAGAATGGAGG | |
| 121917770 | NM_003361.3(UMOD): | CACAYTGACACATGTGGCCAGGG, | Familial juvenile gout |
| | c.383A > G (p.Asn128Ser) | CCACAYTGACACATGTGGCCAGG | |
| 121909008 | NM_000492.3(CFTR): | CACATAAYACGAACTGGTGCTGG | Cystic fibrosis |
| | c.2738A > G (p.Tyr913Cys) | | |
| 137852819 | NM_003688.3(CASK): | CACAGYGGGTCCCTGTCTCCTGG, | FG syndrome 4 |
| | c.2740T > C (p.Trp914Arg) | ACAGYGGGTCCCTGTCTCCTGGG | |
| 74315320 | NM_024009.2(GJB3): | CAAYGATGAGCTTGAAGATGAGG | Deafness, autosomal recessive |
| | c.421A > G (p.Ile141Val) | | |
| 80356747 | NM_001701.3(BAAT): | CAAYGAAGAGGAATTGCCCCTGG | Atypical hemolytic-uremic |
| | c.967A > G (p.Ile323Val) | | syndrome 1 |
| 180177324 | NM_012203.1(GRHPR): | CAAGTYGTTAGCTGCCAACAAGG | Primary hyperoxaluria, type II |
| | c.934A > G (p.Asn312Asp) | | |
| 281860274 | NM_005211.3(CSF1R): | CAAGAYTGGGGACTTCGGGCTGG | Hereditary diffuse |
| | c.2381T > C (p.Ile794Thr) | | leukoencephalopathy with |
| | | | spheroids |
| 398122908 | NM_005334.2(HCFC1): | CAAGAYGGCGGCTCCCAGGGAGG | Mental retardation 3, X-linked |
| | c.−970T > C | | |
| 548076633 | NM_002693.2(POLG): | CAAGAGGYTGGTGATCTGCAAGG | not provided |
| | c.3470A > G (p.Asn1157Ser) | | |
| 120074146 | NM_000019.3(ACAT1): | CAAGAAYAGTAGGTAAGGCCAGG | Deficiency of acetyl-CoA |
| | c.935T > C (p.Ile312Thr) | | acetyltransferase |
| 397514489 | NM_005340.6(HINT1): | CAAGAAAYGTGCTGCTGATCTGG, | Gamstorp-Wohlfart syndrome |
| | c.250T > C (p.Cys84Arg) | AAGAAAYGTGCTGCTGATCTGGG | |
| 587783539 | NM_178151.2(DCX): | CAAAATAYGGAACTTGATTTTGG | Heterotopia |
| | c.2T > C (p.Met1Thr) | | |
| 104894765 | NM_005448.2(BMP15): | ATTGAAAYAGAGTAACAAGAAGG | Ovarian dysgenesis 2 |
| | c.704A > G (p.Tyr235Cys) | | |
| 137852429 | NM_000132.3(F8): | ATGYTGGAGGCTTGGAACTCTGG | Hereditary factor VIII |
| | c.1892A > G (p.Asn631Ser) | | deficiency disease |
| 72558441 | NM_000531.5(OTC):c.779T | ATGTATYAATTACAGACACTTGG | not provided |
| | >C (p.Leu260Ser) | | |
| 398123765 | NM_003494.3(DYSF): | ATGGYAAGGAGCAAGGGAGCAGG | Limb-girdle muscular |
| | c.1284 + 2T > C | | dystrophy, type 2B |
| 387906924 | NM_020191.2(MRPS22): | ATCYTAGGGTAAGGTGACTTAGG | Combined oxidative |
| | c.644T > C (p.Leu215Pro) | | phosphorylation deficiency 5 |
| 397518039 | NM_206933.2(USH2A): | ATCYAAAGCAAAAGACAAGCAGG | Retinitis pigmentosa, Usher |
| | c.8559 − 2A > G | | syndrome, type 2A |
| 5742905 | NM_000071.2(CBS): | ATCAYTGGGGTGGATCCCGAAGG, | Homocystinuria due to CBS |
| | c.833T > C (p.Ile278Thr) | TCAYTGGGGTGGATCCCGAAGGG | deficiency, Homocystinuria, |
| | | | pyridoxine-responsive |
| 397507473 | NM_004333.4(BRAF): | ATCATYTGGAACAGTCTACAAGG, | Cardiofaciocutaneous |
| | c.1403T > C (p.Phe468Ser) | TCATYTGGAACAGTCTACAAGGG | syndrome, Rasopathy |
| 786204056 | NM_000264.3(PTCH1): | TCATTGYGAGTGTATTATAAGGG, | Gorlin syndrome |
| | c.3168 + 2T > C | ATCATTGYGAGTGTATTATAAGG, | |
| | | CATTGYGAGTGTATTATAAGGGG | |
| 72558484 | NM_000531.5(OTC): | ATCATGGYAAGCAAGAAACAAGG | not provided |
| | c.1005 + 2T > C | | |
| 199473074 | NM_000335.4(SCN5A): | ATAYAGTTTTCAGGGCCCGGAGG, | Brugada syndrome |
| | c.688A > G (p.Ile230Val) | CTGATAYAGTTTTCAGGGCCCGG | |
| 111033273 | NM_206933.2(USH2A): | ATATAGAYGCCTCTGCTCCCAGG | Usher syndrome, type 2A |
| | c.1606T > C (p.Cys536Arg) | | |
| 72556290 | NM_000531.5(OTC): | ATAGTGTYCCTAAAAGGCACGGG | not provided |
| | c.542A > G (p.Glu181Gly) | | |
| 121918711 | NM_004612.3(TGFBR1): | ATAGATGYCAGCACGTTTGAAGG | Loeys-Dietz syndrome 1 |
| | c.1199A > G (p.Asp400Gly) | | |
| 104886288 | NM_000495.4(COL4A5): | AGTAYGTGAAGCTCCAGCTGTGG | Alport syndrome, X-linked |
| | c.4699T > C (p.Cys1567Arg) | | recessive |
| 144637717 | NM_016725.2(FOLR1): | CTTCAGGYGAGGGCTGGGGTGGG, | not provided |
| | c.493 + 2T > C | AGGYGAGGGCTGGGGTGGGCAGG | |
| 72558492 | NM_000531.5(OTC): | AGGTGAGYAATCTGTCAGCAGGG | not provided |
| | c.1034A > G (p.Tyr345Cys) | | |
| 62638745 | NM_000121.3(EPOR): | AGGGYTGGAGTAGGGGCCATCGG | Acute myeloid leukemia, M6 |
| | c.1460A > G (p.Asn487Ser) | | type, Familial erythrocytosis, 1 |
| 387907021 | NM_031427.3(DNAL1): | AGGGAYTGCCTACAAACACCAGG | Kartagener syndrome, Ciliary |
| | c.449A > G (p.Asn150Ser) | | dyskinesia, primary, 16 |
| 397514488 | NM_001161581.1(POC1A): | AGCYGTGGGACAAGAGCAGCCGG | Short stature, onychodysplasia, |
| | c.398T > C (p.Leu133Pro) | | facial dysmorphism, and |
| | | | hypotrichosis |
| 154774633 | NM_017882.2(CLN6): | AGCYGGTATTCCCTCTCGAGTGG | Adult neuronal ceroid |
| | c.200T > C (p.Leu67Pro) | | lipofuscinosis |
| 111033700 | NM_000155.3(GALT): | AGCYGGGTGCCCAGTACCCTTGG | Deficiency of UDPglucose- |
| | c.482T > C (p.Leu161Pro) | | hexose-1-phosphate |
| | | | uridylyltransferase |
| 128621198 | NM_000061.2(BTK): | GAGCYGGGGACTGGACAATTTGG, | X-linked agammaglobulinemia |
| | c.1223T > C (p.Leu408Pro) | AGCYGGGGACTGGACAATTTGGG | |
| 137852611 | NM_000211.4(ITGB2): | AGCYAGGTGGCGACCTGCTCCGG | Leukocyte adhesion deficiency |
| | c.446T > C (p.Leu149Pro) | | |
| 121908838 | NM_003722.4(TP63): | AGCTTYTTTGTAGACAGGCATGG | Split-hand/foot malformation 4 |
| | c.697A > G (p.Lys233Glu) | | |
| 397515869 | NM_000169.2(GLA): | AGCTGTGYGATGAAGCAGGCAGG | not specified |
| | c.1153A > G (p.Thr385Ala) | | |
| 118204064 | NM_000237.2(LPL): | GCTGGAYCGAGGCCTTAAAAGGG, | Hyperlipoproteinemia, type I |
| | c.548A > G (p.Asp183Gly) | AGCTGGAYCGAGGCCTTAAAAGG | |
| 128620186 | NM_000061.2(BTK): | AGCTAYGGCCGCAGTGATTCTGG | X-linked agammaglobulinemia |
| | c.2T > C (p.Met1Thr) | | |
| 786204132 | NM_014946.3(SPAST): | ATTGYCTTCCCATTCCCAGGTGG, | Spastic paraplegia 4, |
| | c.1165A > G (p.Thr389Ala) | AGCATTGYCTTCCCATTCCCAGG | autosomal dominant |
| 199473661 | NM_000218.2(KCNQ1): | CAGCAAGBACGTGGGCCTCTGGG, | Congenital long QT syndrome, |
| | c.550T > C (p.Tyr184His) | AGCAAGBACGTGGGCCTCTGGGG, | Cardiac arrhythmia |
| | | GCAAGBACGTGGGCCTCTGGGGG | |
| 387907129 | NM_024599.5(RHBDF2): | AGAYTGTGGATCCGCTGGCCCGG | Howel-Evans syndrome |
| | c.557T > C (p.Ile186Thr) | | |
| 387906702 | NM_006306.3(SMC1A): | AGAYTGGTGTGCGCAACATCCGG | Congenital muscular |
| | c.2351T > C (p.Ile784Thr) | | hypertrophy-cerebral |
| | | | syndrome |
| 193929348 | NM_000525.3(KCNJ11): | AGAYGAGGGTCTCAGCCCTGCGG | Permanent neonatal diabetes |
| | c.544A > G (p.Ile182Val) | | mellitus |
| 121908934 | NM_004086.2(COCH): | AGATAYGGCTTCTAAACCGAAGG | Deafness, autosomal dominant |
| | c.1535T > C (p.Met512Thr) | | 9 |
| 397514377 | NM_000060.3(BTD): | AGAGGYTGTGTTTACGGTAGCGG | Biotinidase deficiency |
| | c.641A > G (p.Asn214Ser) | | |
| 72552295 | NM_000531.5(OTC): | AGAAGAYGCTGTTTAATCTGAGG | not provided |
| | c.2T > C (p.Met1Thr) | | |
| 201893545 | NM_016247.3(IMPG2): | ACTYTTTGGGATCGACTTCCTGG | Macular dystrophy, |
| | c.370T > C (p.Phe124Leu) | | vitelliform, 5 |
| 121434469 | m.4290T > C | ACTYTGATAGAGTAAATAATAGG | |
| 121918733 | NM_006920.4(SCN1A): | ACTTYTATAGTATTGAATAAAGG, | Severe myoclonic epilepsy in |
| | c.269T > C (p.Phe90Ser) | CTTYTATAGTATTGAATAAAGGG | infancy |
| 121434471 | m.4291T > C | ACTTYGATAGAGTAAATAATAGG | Hypertension, |
| | | | hypercholesterolemia, and |
| | | | hypomagnesemia, |
| | | | mitochondrial |
| 606231289 | NM_001302946.1(TRNT1): | ACTTYATTTGACTACTTTAATGG | Sideroblastic anemia with B- |
| | c.497T > C (p.Leu166Ser) | | cell immunodeficiency, |
| | | | periodic fevers, and |
| | | | developmental delay |
| 63750067 | NM_000517.4(HBA2): | CTTYATTCAAAGACCAGGAAGGG, | Hemoglobin H disease, |
| | c.*92A > G | ACTTYATTCAAAGACCAGGAAGG | nondeletional |
| 121918734 | NM_006920.4(SCN1A): | ACTTTTAYAGTATTGAATAAAGG, | Severe myoclonic epilepsy in |
| | c.272T > C (p.Ile91Thr) | CTTTTAYAGTATTGAATAAAGGG | infancy |
| 137854557 | NM_000267.3(NF1): | ACTTAYAGCTTCTTGTCTCCAGG | Neurofibromatosis, type 1 |
| | c.1466A > G (p.Tyr489Cys) | | |
| 397514626 | NM_018344.5(SLC29A3): | ACTGATAYCAGGTGAGAGCCAGG, | Histiocytosis- |
| | c.607T > C (p.Ser203Pro) | CTGATAYCAGGTGAGAGCCAGGG | lymphadenopathy plus |
| | | | syndrome |
| 118204440 | NM_000512.4(GALNS): | ACGYTGAGCTGGGGCTGCGCGGG, | Mucopolysaccharidosis, MPS- |
| | c.1460A > G (p.Asn487Ser) | CACGYTGAGCTGGGGCTGCGCGG | IV-A |
| 587776843 | NG_012088.1:g.2209A > G | ACCYTATGATCCGCCCGCCTTGG | |
| 137853033 | NM_001080463.1(DYNC2H1): | ACCYGTGAAGGGAACAGAGATGG | Short-rib thoracic dysplasia 3 |
| | c.4610A > G (p.Gln1537Arg) | | with or without polydactyly |
| 28933698 | NM_000435.2(NOTCH3): | TTCACCYGTATCTGTATGGCAGG, | Cerebral autosomal dominant |
| | c.1363T > C (p.Cys455Arg) | ACCYGTATCTGTATGGCAGGTGG | arteriopathy with subcortical |
| | | | infarcts and |
| | | | leukoencephalopathy |
| 587776766 | NM_000463.2(UGT1A1): | ACCYGAGATGCAAAATAGGGAGG, | Crigler Najjar syndrome, type |
| | c.1085 − 2A > G | GTGACCYGAGATGCAAAATAGGG, | 1 |
| | | GGTGACCYGAGATGCAAAATAGG | |
| 587781628 | NM_001128425.1(MUTYH): | ACCYGAGAGGGAGGGCAGCCAGG | Hereditary cancer-predisposing |
| | c.1187 − 2A > G | | syndrome, Carcinoma of colon |
| 61755817 | NM_000322.4(PRPH2): | ACCTGYGGGTGCGTGGCTGCAGG, | Retinitis pigmentosa |
| | c.736T > C (p.Trp246Arg) | CCTGYGGGTGCGTGGCTGCAGGG | |
| 121909184 | NM_001089.2(ABCA3): | ACCGTYGTGGCCCAGCAGGACGG | Surfactant metabolism |
| | c.1702A > G (p.Asn568Asp) | | dysfunction, pulmonary, 3 |
| 121434466 | m.4269A > G | ACAYATTTCTTAGGTTTGAGGGG, | |
| | | GACAYATTTCTTAGGTTTGAGGG, | |
| | | AGACAYATTTCTTAGGTTTGAGG | |
| 794726768 | NM_001165963.1(SCN1A): | ACAYATATCCCTCTGGACATTGG | Severe myoclonic epilepsy in |
| | c.1048A > G (p.Met350Val) | | infancy |
| 28934876 | NM_001382.3(DPAGT1): | ACAYAGTACAGGATTCCTGCGGG, | Congenital disorder of |
| | c.509A > G (p.Tyr170Cys) | GACAYAGTACAGGATTCCTGCGG | glycosylation type 1J |
| 104894749 | NM_000054.4(AVPR2): | ACAYAGGTGCGACGGCCCCAGGG, | Nephrogenic diabetes |
| | c.614A > G (p.Tyr205Cys) | GACAYAGGTGCGACGGCCCCAGG | insipidus, Nephrogenic |
| | | | diabetes insipidus, X-linked |
| 128621205 | NM_000061.2(BTK): | ACATTYGGGCTTTTGGTAAGTGG | X-linked agammaglobulinemia |
| | c.1741T > C (p.Trp581Arg) | | |
| 28940892 | NM_000529.2(MC2R): | ACATGYAGCAGGCGCAGTAGGGG, | ACTH resistance |
| | c.761A > G (p.Tyr254Cys) | GACATGYAGCAGGCGCAGTAGGG, | |
| | | AGACATGYAGCAGGCGCAGTAGG | |
| 794726844 | NM_001165963.1(SCN1A): | ACATAYATCCCTCTGGACATTGG | Severe myoclonic epilepsy in |
| | c.1046A > G (p.Tyr349Cys) | | infancy |
| 587783083 | NM_003159.2(CDKL5): | ACAGTYTTAGGACATCATTGTGG | not provided |
| | c.449A > G (p.Lys150Arg) | | |
| 397514651 | NM_000108.4(DLD): | ACAGTTAYAGGTTCTGGTCCTGG, | Maple syrup urine disease, |
| | c.140T > C (p.Ile47Thr) | GTTAYAGGTTCTGGTCCTGGAGG | type 3 |
| 794727060 | NM_001848.2(COL6A1): | ACAAGGYGAGCGTGGGCTGCTGG, | Ullrich congenital muscular |
| | c.957 + 2T > C | CAAGGYGAGCGTGGGCTGCTGGG | dystrophy, Bethlem myopathy |
| 72554346 | NM_000531.5(OTC): | ACAAGATYGTCTACAGAAACAGG | not provided |
| | c.284T > C (p.Leu95Ser) | | |
| 483353031 | NM_002136.2(HNRNPA1): | AATYTTGGAGGCAGAAGCTCTGG | Chronic progressive multiple |
| | c.841T > C (p.Phe281Leu) | | sclerosis |
| 104894271 | NM_000315.2(PTH): | AATTYGTTTTCTTACAAAATCGG | Hypoparathyroidism familial |
| | c.52T > C (p.Cys18Arg) | | isolated |
| 267608260 | NM_015599.2(PGM3): | AATGTYGGCACCATCCTGGGAGG | Immunodeficiency 23 |
| | c.248T > C (p.Leu83Ser) | | |
| 267606900 | NM_018109.3(MTPAP): | AATGGATYCTGAATGTACAGAGG | Ataxia, spastic, 4, autosomal |
| | c.1432A > G (p.Asn478Asp) | | recessive |
| 796053169 | NM_021007.2(SCN2A): | AATAAAGYAGAATATCGTCAAGG | not provided |
| | c.387 − 2A > G | | |
| 104894937 | NM_000116.4(TAZ): | AAGYGTGTGCCTGTGTGCCGAGG | 3-Methylglutaconic aciduria |
| | c.352T > C (p.Cys118Arg) | | type 2 |
| 104893911 | NM_001018077.1(NR3C1): | AAGYGATTGCAGCAGTGAAATGG | Pseudohermaphroditism, |
| | c.1712T > C (p.Val571Ala) | | female, with hypokalemia, due |
| | | | to glucocorticoid resistance |
| 397514472 | NM_004813.2(PEX16): | AAGYAGATTTTCTGCCAGGTGGG, | Peroxisome biogenesis |
| | c.992A > G (p.Tyr331Cys) | GAAGYAGATTTTCTGCCAGGTGG, | disorder 8B |
| | | GTAGAAGYAGATTTTCTGCCAGG | |
| 121918407 | NM_001083112.2(GPD2): | AAGTYTGATGCAGACCAGAAAGG | Diabetes mellitus type 2 |
| | c.1904T > C (p.Phe635Ser) | | |
| 63751110 | NM_000251.2(MSH2): | AAGGAAYGTGTTTTACCCGGAGG | Hereditary Nonpolyposis |
| | c.595T > C (p.Cys199Arg) | | Colorectal Neoplasms |
| 119450945 | NM_000026.2(ADSL): | AAGAYGGTGACAGAAAAGGCAGG | Adenylosuccinate lyase |
| | c.674T > C (p.Met225Thr) | | deficiency |
| 113993988 | NM_002863.4(PYGL): | AAGAAYATGCCCAAAACATCTGG | Glycogen storage disease, type |
| | c.2461T > C (p.Tyr821His) | | VI |
| 119485091 | NM_022041.3(GAN): | AAGAAAAYCTACGCCATGGGTGG, | Giant axonal neuropathy |
| | c.1268T > C (p.Ile423Thr) | AAAAYCTACGCCATGGGTGGAGG | |
| 137852419 | NM_000132.3(F8): | AACYAGAGTAATAGCGGGTCAGG | Hereditary factor VIII |
| | c.1660A > G (p.Ser554Gly) | | deficiency disease |
| 121964967 | NM_000071.2(CBS): | GAACTYGGTCCTGCGGGATGGGG, | Homocystinuria, pyridoxine- |
| | c.1150A > G (p.Lys384Glu) | AACTYGGTCCTGCGGGATGGGGG, | responsive |
| | | GGAACTYGGTCCTGCGGGATGGG, | |
| | | AGGAACTYGGTCCTGCGGGATGG | |
| 137852376 | NM_000132.3(F8): | AACAGAYAATGTCAGACAAGAGG | Hereditary factor VIII |
| | c.1754T > C (p.Ile585Thr) | | deficiency disease |
| 121917930 | NM_006920.4(SCN1A): | AACAAYGGTGGAACCTGAGAAGG | Generalized epilepsy with |
| | c.3577T > C (p.Trp1193Arg) | | febrile seizures plus, type 1, |
| | | | Generalized epilepsy with |
| | | | febrile seizures plus, type 2 |
| 28939717 | NM_003907.2(EIF2B5): | AAATGYTTCCTGTACACCTGTGG | Leukoencephalopathy with |
| | c.271A > G (p.Thr91Ala) | | vanishing white matter |
| 80357276 | NM_007294.3(BRCA1): | AAATATGYGGTCACACTTTGTGG | Familial cancer of breast, |
| | c.122A > G (p.His41Arg) | | Breast-ovarian cancer, familial |
| | | | 1 |
| 397515897 | NM_000256.3(MYBPC3): | AAAGGYGGGCCTGGGACCTGAGG | Familial hypertrophic |
| | c.1351 + 2T > C | | cardiomyopathy 4, |
| | | | Cardiomyopathy |
| 397514491 | NM_005340.6(HINT1): | AAAAYGTGTTGGTGCTTGAGGGG, | Gamstorp-Wohlfart syndrome |
| | c.152A > G (p.His51Arg) | GAAAAYGTGTTGGTGCTTGAGGG, | |
| | | AGAAAAYGTGTTGGTGCTTGAGG | |
| 387907164 | NM_020894.2(UVSSA): | AAAATTYGCAAGTATGTCTTAGG, | UV-sensitive syndrome 3 |
| | c.94T > C (p.Cys32Arg) | AAATTYGCAAGTATGTCTTAGGG | |
| 118161496 | NM_025152.2(NUBPL): | TGGTTCYAATGGATGTCTGCTGG, | Mitochondrial complex I |
| | c.815 − 27T > C | GGTTCYAATGGATGTCTGCTGGG | deficiency |
| 764313717 | NM_005609.2(PYGM): | TGGCTGYCAGGGACCCAGCAAGG, | |
| | c.425528del | CTGYCAGGGACCCAGCAAGGAGG | |
| 28934568 | NM_003242.5(TGFBR2): | AGTTCCYGACGGCTGAGGAGCGG | Loeys-Dietz syndrome 2 |
| | c.923T > C (p.Leu308Pro) | | |
| 121913461 | NM_007313.2(ABL1): | CCAGYACGGGGAGGTGTACGAGG, | |
| | c.814T > C (p.Tyr272His) | CAGYACGGGGAGGTGTACGAGGG | |
| 377750405 | NM_173551.4(ANKS6): | AGGGCYGTCGGACCTTCGAGTGG, | Nephronophthisis 16 |
| | c.1322A > G (p.Gln441Arg) | GGGCYGTCGGACCTTCGAGTGGG, | |
| | | GGCYGTCGGACCTTCGAGTGGGG | |
| 57639980 | NM_001927.3(DES): | ATTCCCYGATGAGGCAGATGCGG, | Myofibrillar myopathy 1 |
| | c.1034T > C (p.Leu345Pro) | TTCCCYGATGAGGCAGATGCGGG | |
| 147391618 | NM_020320.3(RARS2): | ATACCYGGCAAGCAATAGCGCGG | Pontocerebellar hypoplasia |
| | c.35A > G (p.Gln12Arg) | | type 6 |
| 182650126 | NM_002977.3(SCN9A): | GTAAYTGCAAGATCTACAAAAGG | Small fiber neuropathy |
| | c.2215A > G (p.Ile739Val) | | |
| 80358278 | NM_004700.3(KCNQ4): | ACATYGACAACCATCGGCTATGG | DFNA 2 Nonsyndromic |
| | c.842T > C (p.Leu281Ser) | | Hearing Loss |
| 786204012 | NM_005957.4(MTHFR): | GACCYGCTGCCGTCAGCGCCTGG | Homocysteinemia due to |
| | c.388T > C (p.Cys130Arg) | | MTHFR deficiency |
| 786204037 | NM_005957.4(MTHFR): | TCCCACYGGACAACTGCCTCTGG | Homocysteinemia due to |
| | c.1883T > C (p.Leu628Pro) | | MTHFR deficiency |
| 202147607 | NM_000140.3(FECH): | GTAGAYACCTTAGAGAACAATGG | Erythropoietic protoporphyria |
| | c.1137 + 3A > G | | |
| 122456136 | NM_005183.3(CACNA1F): | TGCCAYTGCTGTGGACAACCTGG | |
| | c.2267T > C (p.Ile756Thr) | | |
| 786204851 | NM_007374.2(SIX6): | GTCGCYGCCCGTGGCCCCTGCGG | Cataract, microphthalmia and |
| | c.110T > C (p.Leu37Pro) | | nystagmus |
| 794728167 | NM_000138.4(FBN1): | ATTGGYACGTGATCCATCCTAGG | Thoracic aortic aneurysms and |
| | c.1468 + 2T > C | | aortic dissections |
| 121964909 | NM_000027.3(AGA): | GACGGCYCTGTAGGCTTTGGAGG | Aspartylglycosaminuria |
| | c.214T > C (p.Ser72Pro) | | |
| 121964978 | NM_000170.2(GLDC): | CGGCCAYGCAGTCCTGTGCCAGG, | Non-ketotic hyperglycinemia |
| | c.2T > C (p.Met1Thr) | GGCCAYGCAGTCCTGTGCCAGGG | |
| 121965008 | NM_000398.6(CYB5R3): | CTGCYGGTCTACCAGGGCAAAGG | METHEMOGLOBINEMIA, |
| | c.446T > C (p.Leu149Pro) | | TYPE I |
| 121965064 | NM_000128.3(F11): | TGATYTCTTGGGAGAAGAACTGG | Hereditary factor XI deficiency |
| | c.901T > C (p.Phe301Leu) | | disease |
| 45517398 | NM_000548.3(TSC2): | GCCCYGCACGCAAATGTGAGTGG, | Tuberous sclerosis syndrome |
| | c.5150T > C (p.Leu1717Pro) | CCCYGCACGCAAATGTGAGTGGG | |
| 786205857 | NM_015662.2(IFT172): | TTGTGCYAGGAAGTTATGACAGG | RETINITIS PIGMENTOSA |
| | c.770T > C (p.Leu257Pro) | | 71 |
| 786205904 | NM_001135669.1(XPR1): | GCGTTYACGTGTCCCCCCTTTGG, | BASAL GANGLIA |
| | c.653T > C (p.Leu218Ser) | CGTTYACGTGTCCCCCCTTTGGG | CALCIFICATION, |
| | | | IDIOPATHIC, 6 |
| 104893704 | NM_000388.3(CASR): | ACGCTYTCAAGGTGGCTGCCCGG, | Hypercalciuric hypercalcemia |
| | c.2641T > C (p.Phe881Leu) | CGCTYTCAAGGTGGCTGCCCGGG | |
| 104893747 | NM_198159.2(MITF): | ACTTYCCCTTATTCCATCCACGG, | Waardenburg syndrome type |
| | c.1195T > C (p.Ser399Pro) | CTTYCCCTTATTCCATCCACGGG | 2A |
| 104893770 | NM_000539.3(RHO): | CATGYTTCTGCTGATCGTGCTGG, | Retinitis pigmentosa 4 |
| | c.133T > C (p.Phe45Leu) | ATGYTTCTGCTGATCGTGCTGGG | |
| 28937596 | NM_003907.2(EIF2B5): | AGGCCYGGAGCCCTGTTTTTAGG | Leukoencephalopathy with |
| | c.1882T > C (p.Trp628Arg) | | vanishing white matter |
| 104893876 | NM_001151.3(SLC25A4): | GCAGCYCTTCTTAGGGGGTGTGG | Autosomal dominant |
| | c.293T > C (p.Leu98Pro) | | progressive external |
| | | | ophthalmoplegia with |
| | | | mitochondrial DNA deletions |
| | | | 2 |
| 104893883 | NM_006005.3(WFS1): | ACCATCCYGGAGGGCCGCCTGGG | WFS1-Related Disorders |
| | c.2486T > C (p.Leu829Pro) | | |
| 104893962 | NM_000165.4(GJA1): | CTACYCAACTGCTGGAGGGAAGG | Oculodentodigital dysplasia |
| | c.52T > C (p.Ser18Pro) | | |
| 104893978 | NM_000434.3(NEU1): | CCTCCYGGCGCTACGGAAGTGGG, | Sialidosis, type II |
| | c.718T > C (p.Trp240Arg) | GCCTCCYGGCGCTACGGAAGTGG, | |
| | | CTCCYGGCGCTACGGAAGTGGGG | |
| 104894092 | NM_002546.3(TNFRSF11B): | TAGAGYTCTGCTTGAAACATAGG | Hyperphosphatasemia with |
| | c.349T > C (p.Phe117Leu) | | bone disease |
| 104894135 | NM_000102.3(CYP17A1): | CATCGCGYCCAACAACCGTAAGG, | Complete combined 17-alpha- |
| | c.316T > C (p.Ser106Pro) | ATCGCGYCCAACAACCGTAAGGG | hydroxylase/17,20-lyase |
| | | | deficiency |
| 104894151 | NM_000102.3(CYP17A1): | AGCTCTYCCTCATCATGGCCTGG | Combined partial 17-alpha- |
| | c.1358T > C (p.Phe453Ser) | | hydroxylase/17,20-lyase |
| | | | deficiency |
| 36015961 | NM_000518.4(HBB): | TGTGTGCYGGCCCATCACTTTGG | Beta thalassemia intermedia |
| | c.344T > C (p.Leu115Pro) | | |
| 104894472 | NM_152443.2(RDH12): | TCCYCGGTGGCTCACCACATTGG | Leber congenital amaurosis 13 |
| | c.523T > C (p.Ser175Pro) | | |
| 104894587 | NM_004870.3(MPDU1): | TTCCYGGTCATGCACTACAGAGG | Congenital disorder of |
| | c.356T > C (p.Leu119Pro) | | glycosylation type 1F |
| 104894588 | NM_004870.3(MPDU1): | AATAYGGCGGCCGAGGCGGACGG | Congenital disorder of |
| | c.2T > C (p.Met1Thr) | | glycosylation type 1F |
| 104894626 | NM_000304.3(PMP22): | TAGCAAYGGATCGTGGGCAATGG | Charcot-Marie-Tooth disease, |
| | c.82T > C (p.Trp28Arg) | | type IE |
| 104894631 | NM_018129.3(PNPO): | ACCTYAACTCTGGGACCTGCTGG | Pyridoxal 5-phosphate- |
| | c.784T > C (p.Ter262Gln) | | dependent epilepsy |
| 104894703 | NM_032551.4(KISS1R): | GCCCTGCYGTACCCGCTGCCCGG, | |
| | c.305T > C (p.Leu102Pro) | TGCYGTACCCGCTGCCCGGCTGG | |
| 104894826 | NM_000166.5(GJB1): | ATGYCATCAGCGTGGTGTTCCGG | Dejerine-Sottas disease, X- |
| | c.407T > C (p.Val136Ala) | | linked hereditary motor and |
| | | | sensory neuropathy |
| 104894859 | NM_001122606.1(LAMP2): | CAGCTACYGGGATGCCCCCCTGG, | Danon disease |
| | c.961T > C (p.Trp321Arg) | AGCTACYGGGATGCCCCCCTGGG | |
| 104894931 | NM_006517.4(SLC16A2): | TGAGCYGGTGGGCCCAATGCAGG | Allan-Herndon-Dudley |
| | c.1313T > C (p.Leu438Pro) | | syndrome |
| 104894935 | NM_000330.3(RS1): | TTACTTCYCTTTGGCTATGAAGG | Juvenile retinoschisis |
| | c.38T > C (p.Leu13Pro) | | |
| 104895217 | NM_001065.3(TNFRSF1A): | TGCYGTACCAAGTGCCACAAAGG | TNF receptor-associated |
| | c.175T > C (p.Cys59Arg) | | periodic fever syndrome |
| | | | (TRAPS) |
| 143889283 | NM_003793.3(CTSF): | CTCCAYACTGAGCTGTGCCACGG | Ceroid lipofuscinosis, |
| | c.692A > G (p.Tyr231Cys) | | neuronal, 13 |
| 122459147 | NM_001159702.2(FHL1): | GGGGYGCTTCAAGGCCATTGTGG | Myopathy, reducing body, X- |
| | c.310T > C (p.Cys104Arg) | | linked, childhood-onset |
| 74552543 | NM_020184.3(CNNM4): | AAGCTCCYGGACTTTTTTCTGGG | Cone-rod dystrophy |
| | c.971T > C (p.Leu324Pro) | | amelogenesis imperfecta |
| 199476117 | m.10158T > C | AAAYCCACCCCTTACGAGTGCGG | Leigh disease, Leigh syndrome |
| | | | due to mitochondrial complex |
| | | | I deficiency, Mitochondrial |
| | | | complex I deficiency |
| 794727808 | NM_020451.2(SEPN1): | TTCCGGYGAGTGGGCCACACTGG | Congenital myopathy with |
| | c.872 + 2T > C | | fiber type disproportion, |
| | | | Eichsfeld type congenital |
| | | | muscular dystrophy |
| 140547520 | NM_005022.3(PFN1): | CACCTYCTTTGCCCATCAGCAGG | Amyotrophic lateral sclerosis |
| | c.350A > G (p.Glu117Gly) | | 18 |
| 397514359 | NM_000060.3(BTD): | TCACCGCYTCAATGACACAGAGG | Biotinidase deficiency |
| | c.445T > C (p.Phe149Leu) | | |
| 207460001 | m.15197T > C | CTAYCCGCCATCCCATACATTGG | Exercise intolerance |
| 397514406 | NM_000060.3(BTD): | TTCACCCYGGTCCCTGTCTGGGG | Biotinidase deficiency |
| | c.1214T > C (p.Leu405Pro) | | |
| 397514516 | NM_006177.3(NRL): | GAGGCCAYGGAGCTGCTGCAGGG | Retinitis pigmentosa 27 |
| | c.287T > C (p.Met96Thr) | | |
| 72554312 | NM_000531.5(OTC): | CTCACTCYAAAAAACTTTACCGG | Ornithine |
| | c.134T > C (p.Leu45Pro) | | carbamoyltransferase |
| | | | deficiency |
| 397514569 | NM_178012.4(TUBB2B): | GGTCCYGGATGTGGTGAGGAAGG | Polymicrogyria, asymmetric |
| | c.350T > C (p.Leu117Pro) | | |
| 397514571 | NM_000431.3(MVK): | CGGCYTCAACCCCACAGCAATGG, | Porokeratosis, disseminated |
| | c.122T > C (p.Leu41Pro) | GGCYTCAACCCCACAGCAATGGG | superficial actinic 1 |
| 794728390 | NM_000238.3(KCNH2): | GCCATCCYGGGTATGGGGTGGGG, | Cardiac arrhythmia |
| | c.2396T > C (p.Leu799Pro) | CCATCCYGGGTATGGGGTGGGGG, | |
| | | CATCCYGGGTATGGGGTGGGGGG | |
| 397514713 | NM_001199107.1(TBC1D24): | GGTCTYTGACGTCTTCCTGGTGG | Early infantile epileptic |
| | c.686T > C (p.Phe229Ser) | | encephalopathy 16 |
| 397514719 | NM_080605.3(B3GALT6): | CGCYGGCCACCAGCACTGCCAGG | Spondyloepimetaphyseal |
| | c.193A > G (p.Ser65Gly) | | dysplasia with joint laxity |
| 730880608 | NM_000256.3(MYBPC3): | GAGYGCCGCCTGGAGGTGCGAGG | Cardiomyopathy |
| | c.3796T > C (p.Cys1266Arg) | | |
| 397515329 | NM_001382.3(DPAGT1): | AATCCYGTACTATGTCTACATGG, | Congenital disorder of |
| | c.503T > C (p.Leu168Pro) | ATCCYGTACTATGTCTACATGGG, | glycosylation type 1J |
| | | TCCYGTACTATGTCTACATGGGG | |
| 397515465 | NM_018127.6(ELAC2): | ATAYTTTCTGGTCCATTGAAAGG | Combined oxidative |
| | c.460T > C (p.Phe154Leu) | | phosphorylation deficiency 17 |
| 397515557 | NM_005211.3(CSF1R): | CATCTYTGACTGTGTCTACACGG | Hereditary diffuse |
| | c.2483T > C (p.Phe828Ser) | | leukoencephalopathy with |
| | | | spheroids |
| 397515599 | NM_194248.2(OTOF): | AGGTGCYGTTCTGGGGCCTACGG, | Deafness, autosomal recessive |
| | c.3413T > C (p.Leu1138Pro) | GGTGCYGTTCTGGGGCCTACGGG | 9 |
| 397515766 | NM_000138.4(FBN1): | GGACAAYGTAGAAATACTCCTGG | Marfan syndrome |
| | c.2341T > C (p.Cys781Arg) | | |
| 565779970 | NM_001429.3(EP300): | CTTAYTACAGTTACCAGAACAGG | Rubinstein-Taybi syndrome 2 |
| | c.3573T > A (p.Tyr1191Ter) | | |
| 786200938 | NM_080605.3(B3GALT6): | AGCTTCAYGGCGCCCGCGCCGGG, | Spondyloepimetaphyseal |
| | c.1A > G (p.Met1Val) | TCAYGGCGCCCGCGCCGGGCCGG | dysplasia with joint laxity |
| 28942087 | NM_000229.1(LCAT): | ATCTCTCYTGGGGCTCCCTGGGG, | Norum disease |
| | c.698T > C (p.Leu233Pro) | TCTCYTGGGGCTCCCTGGGGTGG | |
| 128621203 | NM_000061.2(BTK): | TCGGCCYGTCCAGGTGAGTGTGG | X-linked agammaglobulinemia |
| | c.1625T > C (p.Leu542Pro) | | with growth hormone |
| | | | deficiency |
| 397515412 | NM_006383.3(CIB2): | CTTCAYCTGCAAGGAGGACCTGG | Deafness, autosomal recessive |
| | c.368T > C (p.Ile123Thr) | | 48 |
| 193929364 | NM_000352.4(ABCC8): | AAGCYGCTAATTGGTAGGTGAGG | Permanent neonatal diabetes |
| | c.404T > C (p.Leu135Pro) | | mellitus |
| 730880872 | NM_000257.3(MYH7): | TCGAGAYCTTCGATGTGAGTTGG, | Cardiomyopathy |
| | c.1400T > C (p.Ile467Thr) | CGAGAYCTTCGATGTGAGTTGGG | |
| 80356474 | NM_002977.3(SCN9A): | AAGATCAYTGGTAACTCAGTAGG, | Primary erythromelalgia |
| | c.2543T > C (p.Ile848Thr) | AGATCAYTGGTAACTCAGTAGGG, | |
| | | GATCAYTGGTAACTCAGTAGGGG | |
| 80356489 | NM_001164277.1(SLC37A4): | GGGCYGGCCCCCATGTGGGAAGG | Glucose-6-phosphate transport |
| | c.352T > C (p.Trp118Arg) | | defect |
| 80356536 | NM_152296.4(ATP1A3): | GCCCYTCCTGCTGTTCATCATGG | Dystonia 12 |
| | c.2338T > C (p.Phe780Leu) | | |
| 80356596 | NM_194248.2(OTOF): | GATGCYGGTGTTCGACAACCTGG | Deafness, autosomal recessive |
| | c.3032T > C (p.Leu1011Pro) | | 9, Auditory neuropathy, |
| | | | autosomal recessive, 1 |
| 80356689 | NM_000083.2(CLCN1): | AGGAGYGCTATTTAGCATCGAGG | Myotonia congenita |
| | c.857T > C (p.Val286Ala) | | |
| 118203884 | m.4409T > C | AGGYCAGCTAAATAAGCTATCGG | Mitochondrial myopathy |
| 587777625 | NM_173596.2(SLC39A5): | AGAACAYGCTGGGGCTTTTGCGG | Myopia 24, autosomal |
| | c.911T > C (p.Met304Thr) | | dominant |
| 587783087 | NM_003159.2(CDKL5): | ATTCYTGGGGAGCTTAGCGATGG | not provided |
| | c.602T > C (p.Leu201Pro) | | |
| 118203951 | NM_013319.2(UBIAD1): | TCTGGCYCCTTTCTCTACACAGG, | Schnyder crystalline corneal |
| | c.511T > C (p.Ser171Pro) | GGCYCCTTTCTCTACACAGGAGG | dystrophy |
| 118204017 | NM_000018.3(ACADVL): | TCGCATCYTCCGGATCTTTGAGG, | Very long chain acyl-CoA |
| | c.1372T > C (p.Phe458Leu) | CGCATCYTCCGGATCTTTGAGGG, | dehydrogenase deficiency |
| | | GCATCYTCCGGATCTTTGAGGGG | |
| 397518466 | NM_000833.4(GRIN2A): | CTAYGGGCAGAGTGGGCTATTGG | Focal epilepsy with speech |
| | c.2T > C (p.Met1Thr) | | disorder with or without |
| | | | mental retardation |
| 118204069 | NM_000237.2(LPL): | GGACYGGCTGTCACGGGCTCAGG | Hyperlipoproteinemia, type I |
| | c.337T > C (p.Trp113Arg) | | |
| 118204080 | NM_000237.2(LPL): | GTGAYTGCAGAGAGAGGACTTGG | Hyperlipoproteinemia, type I |
| | c.755T > C (p.Ile252Thr) | | |
| 118204111 | NM_000190.3(HMBS): | GCTTCGCYGCATCGCTGAAAGGG | Acute intermittent porphyria |
| | c.739T > C (p.Cys247Arg) | | |
| 80357438 | NM_007294.3(BRCA1): | AAATCTYAGAGTGTCCCATCTGG | Familial cancer of breast, |
| | c.65T > C (p.Leu22Ser) | | Breast-ovarian cancer, familial |
| | | | 1, Hereditary cancer- |
| | | | predisposing syndrome |
| 139877390 | NM_001040431.2(COA3): | CCAYCTGGGGAGGTAGGTTCAGG | |
| | c.215A > G (p.Tyr72Cys) | | |
| 793888527 | NM_005859.4(PURA): | GACCAYTGCGCTGCCCGCGCAGG, | not provided, Mental |
| | c.563T > C (p.Ile188Thr) | ACCAYTGCGCTGCCCGCGCAGGG, | retardation, autosomal |
| | | CCAYTGCGCTGCCCGCGCAGGGG | dominant 31 |
| 561425038 | NM_002878.3(RAD51D): | CGCCCAYGTTCCCCGCAGGCCGG | Hereditary cancer-predisposing |
| | c.1A > G (p.Met1Val) | | syndrome |
| 121907934 | NM_024105.3(ALG12): | TCCYGCTGGCCCTCGCGGCCTGG | Congenital disorder of |
| | c.473T > C (p.Leu158Pro) | | glycosylation type 1G |
| 80358207 | NM_153212.2(GJB4): | CCTCATCYTCAAGGCCGCCGTGG | Erythrokeratodermia variabilis |
| | c.409T > C (p.Phe137Leu) | | |
| 80358228 | NM_002353.2(TACSTD2): | TCGGCYGCACCCCAAGTTCGTGG | Lattice corneal dystrophy Type |
| | c.557T > C (p.Leu186Pro) | | III |
| 121908076 | NM_138691.2(TMC1): | AGGACCTYGCTGGGAAACAATGG, | Deafness, autosomal recessive |
| | c.1543T > C (p.Cys515Arg) | ACCTYGCTGGGAAACAATGGTGG, | 7 |
| | | CCTYGCTGGGAAACAATGGTGGG | |
| 121908089 | NM_017838.3(NHP2): | GGAGGCTYACGATGAGTGCCTGG, | Dyskeratosis congenita |
| | c.415T > C (p.Tyr139His) | GGCTYACGATGAGTGCCTGGAGG | autosomal recessive 1, |
| | | | Dyskeratosis congenita, |
| | | | autosomal recessive 2 |
| 121908154 | NM_001243133.1(NLRP3): | GGTGCCTYTGACGAGCACATAGG | Familial cold urticaria, |
| | c.926T > C (p.Phe309Ser) | | Chronic infantile neurological, |
| | | | cutaneous and articular |
| | | | syndrome |
| 121908158 | NM_001033855.2(DCLRE1C): | GGCGCTAYGAGTTCTTTCGAGGG, | Histiocytic medullary |
| | c.2T > C (p.Met1Thr) | GCGCTAYGAGTTCTTTCGAGGGG | reticulosis |
| 796052870 | NM_018129.3(PNPO): | CCCCCAYGACGTGCTGGCTGCGG, | not provided |
| | c.2T > C (p.Met1Thr) | CCCCAYGACGTGCTGGCTGCGGG, | |
| | | CCCAYGACGTGCTGGCTGCGGGG | |
| 121908318 | NM_020427.2(SLURP1): | GCAGCCYGGAGCATGGGCTGTGG | Acroerythrokeratoderma |
| | c.43T > C (p.Trp15Arg) | | |
| 121908352 | NM_022124.5(CDH23): | CTCACCTYCAACATCACTGCGGG | Deafness, autosomal recessive |
| | c.5663T > C (p.Phe1888Ser) | | 12 |
| 121908520 | NM_000030.2(AGXT): | CCTGTACYCGGGCTCCCAGAAGG | Primary hyperoxaluria, type I |
| | c.613T > C (p.Ser205Pro) | | |
| 121908618 | NM_004273.4(CHST3): | CGTGCYGGCCTCGCGCATGGTGG | Spondyloepiphyseal dysplasia |
| | c.920T > C (p.Leu307Pro) | | with congenital joint |
| | | | dislocations |
| 11694 | NM_006432.3(NPC2): | TATTCAGYCTAAAAGCAGCAAGG | Niemann-Pick disease type C2 |
| | c.199T > C (p.Ser67Pro) | | |
| 121908739 | NM_000022.2(ADA): | CCTGCYGGCCAACTCCAAAGTGG | Severe combined |
| | c.320T > C (p.Leu107Pro) | | immunodeficiency due to |
| | | | ADA deficiency |
| 80359022 | NM_000059.3(BRCA2): | TGCYTCTTCAACTAAAATACAGG | Familial cancer of breast, |
| | c.7958T > C (p.Leu2653Pro) | | Breast-ovarian cancer, familial |
| | | | 2 |
| 121908902 | NM_003880.3(WISP3): | AAAATCYGTGCCAAGCAACCAGG, | Progressive pseudorheumatoid |
| | c.232T > C (p.Cys78Arg) | AAATCYGTGCCAAGCAACCAGGG, | dysplasia |
| | | AATCYGTGCCAAGCAACCAGGGG | |
| 121908947 | NM_006892.3(DNMT3B): | CAAGTTCYCCGAGGTGAGTCCGG, | Centromeric instability of |
| | c.808T > C (p.Ser270Pro) | AAGTTCYCCGAGGTGAGTCCGGG, | chromosomes 1,9 and 16 and |
| | | AGTTCYCCGAGGTGAGTCCGGGG | immunodeficiency |
| 121909028 | NM_000492.3(CFTR): | AGCCTYTGGAGTGATACCACAGG | Cystic fibrosis |
| | c.3857T > C (p.Phe1286Ser) | | |
| 121909135 | NM_000085.4(CLCNKB): | CTTTGTCYATGGTGAGTCTGGGG | Bartter syndrome type 3 |
| | c.1294T > C (p.Tyr432His) | | |
| 121909143 | NM_001300.5(KLF6): | GGAGCYGCCCTCGCCAGGGAAGG | |
| | c.506T > C (p.Leu169Pro) | | |
| 121909182 | NM_001089.2(ABCA3): | GCACYTGTGATCAACATGCGAGG | Surfactant metabolism |
| | c.302T > C (p.Leu101Pro) | | dysfunction, pulmonary, 3 |
| 121909200 | NM_000503.5(EYA1): | CACTCYCGCTCATTCACTCCCGG | Melnick-Fraser syndrome |
| | c.1459T > C (p.Ser487Pro) | | |
| 121909247 | NM_004970.2(IGFALS): | GGACYGTGGCTGCCCTCTCAAGG | Acid-labile subunit deficiency |
| | c.1618T > C (p.Cys540Arg) | | |
| 121909253 | NM_005570.3(LMAN1): | AGAYGGCGGGATCCAGGCAAAGG | Combined deficiency of factor |
| | c.2T > C (p.Met1Thr) | | V and factor VIII, 1 |
| 121909385 | NM_000339.2(SLC12A3): | CAACCYGGCCCTCAGCTACTCGG | Familial hypokalemia- |
| | c.1868T > C (p.Leu623Pro) | | hypomagnesemia |
| 121909497 | NM_002427.3(MMP13): | TTCTYCGGCTTAGAGGTGACTGG | Spondyloepimetaphyseal |
| | c.224T > C (p.Phe75Ser) | | dysplasia, Missouri type |
| 121909508 | NM_000751.2(CHRND): | AACCYCATCTCCCTGGTGAGAGG | MYASTHENIC |
| | c.188T > C (p.Leu63Pro) | | SYNDROME, |
| | | | CONGENITAL, 3B, FAST- |
| | | | CHANNEL |
| 121909519 | NM_001100.3(ACTA1): | CGAGCYTCGCGTGGCTCCCGAGG | Nemaline myopathy 3 |
| | c.287T > C (p.Leu96Pro) | | |
| 121909572 | NM_000488.3(SERPINC1): | TGGGTGYCCAATAAGACCGAAGG | Antithrombin III deficiency |
| | c.667T > C (p.Ser223Pro) | | |
| 121909677 | NM_000821.6(GGCX): | TATGTYCTCCTACGTCATGCTGG | Pseudoxanthoma elasticum- |
| | c.896T > C (p.Phe299Ser) | | like disorder with multiple |
| | | | coagulation factor deficiency |
| 121909727 | NM_001018077.1(NR3C1): | CTATTGCYTCCAAACATTTTTGG | Glucocorticoid resistance, |
| | c.2209T > C (p.Phe737Leu) | | generalized |
| 139573311 | NM_000492.3(CFTR): | TTCACYTCTAATGGTGATTATGG, | Cystic fibrosis |
| | c.1400T > C (p.Leu467Pro) | TCACYTCTAATGGTGATTATGGG | |
| 121912441 | NM_000454.4(SOD1): | CATCAYTGGCCGCACACTGGTGG | Amyotrophic lateral sclerosis |
| | c.341T > C (p.Ile114Thr) | | type 1 |
| 121912446 | NM_000454.4(SOD1): | CGTTYGGCTTGTGGTGTAATTGG, | Amyotrophic lateral sclerosis |
| | c.434T > C (p.Leu145Ser) | GTTYGGCTTGTGGTGTAATTGGG | type 1 |
| 121912463 | NM_000213.3(ITGB4): | GGCCAGYGTGTGTGTGAGCCTGG | Epidermolysis bullosa with |
| | c.1684T > C (p.Cys562Arg) | | pyloric atresia |
| 121912492 | NM_002292.3(LAMB2): | CCTCAACYGCGAGCAGTGTCAGG | Nephrotic syndrome, type 5, |
| | c.961T > C (p.Cys321Arg) | | with or without ocular |
| | | | abnormalities |
| 397516659 | NM_001399.4(EDA): | GGCCAYGGGCTACCCGGAGGTGG | Hypohidrotic X-linked |
| | c.2T > C (p.Met1Thr) | | ectodermal dysplasia |
| 111033589 | NM_021044.2(DHH): | GTTGCYGGCGCGCCTCGCAGTGG | 46,XY gonadal dysgenesis, |
| | c.485T > C (p.Leu162Pro) | | complete, dhh-related |
| 111033622 | NM_000206.2(IL2RG): | TGGCYGTCAGTTGCAAAAAAAGG | X-linked severe combined |
| | c.343T > C (p.Cys115Arg) | | immunodeficiency |
| 121912613 | NM_001041.3(SI): | ATGCYGGAGTTCAGTTTGTTTGG | Sucrase-isomaltase deficiency |
| | c.1859T > C (p.Leu620Pro) | | |
| 121912619 | NM_016180.4(SLC45A2): | GAGTTTCYCATCTACGAAAGAGG | Oculocutaneous albinism type |
| | c.1082T > C (p.Leu361Pro) | | 4 |
| 61750581 | NM_000552.3(VWF): | CTGCCYCTGATGAGATCAAGAGG | von Willebrand disease, type |
| | c.4837T > C (p.Ser1613Pro) | | 2a |
| 121912653 | NM_000546.5(TP53): | CATCCYCACCATCATCACACTGG | Li-Fraumeni syndrome 1 |
| | c.755T > C (p.Leu252Pro) | | |
| 111033683 | NM_000155.3(GALT): | AGGTCAYGTGCTTCCACCCCTGG | Deficiency of UDPglucose- |
| | c.386T > C (p.Met129Thr) | | hexose-1-phosphate |
| | | | uridylyltransferase |
| 111033752 | NM_000155.3(GALT): | CAGGAGCYACTCAGGAAGGTGGG | Deficiency of UDPglucose- |
| | c.677T > C (p.Leu226Pro) | | hexose-1-phosphate |
| | | | uridylyltransferase |
| 121912729 | NM_000039.1(APOA1): | GCGCTYGGCCGCGCGCCTTGAGG | Familial visceral amyloidosis, |
| | c.593T > C (p.Leu198Ser) | | Ostertag type |
| 769452 | NM_000041.3(APOE): | AACYGGCACTGGGTCGCTTTTGG | |
| | c.137T > C (p.Leu46Pro) | | |
| 121912762 | NM_016124.4(RHD): | ACACYGTTCAGGTATTGGGATGG | |
| | c.329T > C (p.Leu110Pro) | | |
| 111033824 | NM_000155.3(GALT): | CGCCYGACCACGCCGACCACAGG, | Deficiency of UDPglucose- |
| | c.1138T > C (p.Ter380Arg) | GCCYGACCACGCCGACCACAGGG | hexose-1-phosphate |
| | | | uridylyltransferase |
| 111033832 | NM_000155.3(GALT): | TCCYGCGCTCTGCCACTGTCCGG | Deficiency of UDPglucose- |
| | c.980T > C (p.Leu327Pro) | | hexose-1-phosphate |
| | | | uridylyltransferase |
| 730881974 | NM_000455.4(STK11): | GGGAACCYGCTGCTCACCACCGG, | Hereditary cancer-predisposing |
| | c.545T > C (p.Leu182Pro) | AACCYGCTGCTCACCACCGGTGG | syndrome |
| 1064644 | NM_000157.3(GBA): | GGGYCACTCAAGGGACAGCCCGG | Gaucher disease |
| | c.703T > C (p.Ser235Pro) | | |
| 796052090 | NM_138413.3(HOGA1): | GGACCYGCCTGTGGATGCAGTGG | Primary hyperoxaluria, type III |
| | c.533T > C (p.Leu178Pro) | | |
| 121913141 | NM_000208.2(INSR): | CTACCYGGACGGCAGGTGTGTGG | Leprechaunism syndrome |
| | c.779T > C (p.Leu260Pro) | | |
| 121913272 | NM_006218.2(PIK3CA): | GGAACACYGTCCATTGGCATGGG, | Congenital lipomatous |
| | c.1258T > C (p.Cys420Arg) | GAACACYGTCCATTGGCATGGGG | overgrowth, vascular |
| | | | malformations, and epidermal |
| | | | nevi, Neoplasm of ovary, |
| | | | PIK3CA Related Overgrowth |
| | | | Spectrum |
| 61751310 | NM_000552.3(VWF): | GCTCCYGCTGCTCTCCGACACGG | von Willebrand disease, type |
| | c.8317T > C (p.Cys2773Arg) | | 2a |
| 312262799 | NM_024408.3(NOTCH2): | TTCACAYGTCTGTGCATGCCAGG | Alagille syndrome 2 |
| | c.1438T > C (p.Cys480Arg) | | |
| 121913570 | NM_000426.3(LAMA2): | ATCATTCYTTTGGGAAGTGGAGG, | Merosin deficient congenital |
| | c.7691T > C (p.Leu2564Pro) | TCATTCYTTTGGGAAGTGGAGGG | muscular dystrophy |
| 121913640 | NM_000257.3(MYH7): | AACTCCAYGTATAAGCTGACAGG | Familial hypertrophic |
| | c.1046T > C (p.Met349Thr) | | cardiomyopathy 1, |
| | | | Cardiomyopathy |
| 121913642 | NM_000257.3(MYH7): | CATCATGYCCATCCTGGAAGAGG | Dilated cardiomyopathy 1S |
| | c.1594T > C (p.Ser532Pro) | | |
| 119463996 | NM_001079802.1(FKTN): | GTAGTCTYTCATGAGAGGAGTGG | Limb-girdle muscular |
| | c.527T > C (p.Phe176Ser) | | dystrophy- |
| | | | dystroglycanopathy, type C4 |
| 587776456 | NM_002049.3(GATA1): | GCTCAYGAGGGCACAGAGCATGG | GATA-1-related |
| | c.1240T > C (p.Ter414Arg) | | thrombocytopenia with |
| | | | dyserythropoiesis |
| 63750654 | NM_000184.2(HBG2): | ATGCAAAYATCTGTCTGAAACGG | Fetal hemoglobin quantitative |
| | c.−228T > C | | trait locus 1 |
| 587776519 | NM_001999.3(FBN2): | AGCAYTGCAACCACATTGTCAGG | Congenital contractural |
| | c.3725 − 15A > G | | arachnodactyly |
| 78365220 | NM_000402.4(G6PD): | TGCCCYCCACCTGGGGTCACAGG | Anemia, nonspherocytic |
| | c.473T > C (p.Leu158Pro) | | hemolytic, due to G6PD |
| | | | deficiency |
| 63750741 | NM_000179.2(MSH6): | CTGGGGCYGGTATTCATGAAAGG | Hereditary Nonpolyposis |
| | c.1346T > C (p.Leu449Pro) | | Colorectal Neoplasms |
| 587776914 | NM_017565.3(FAM20A): | GTAATCYGCAAAGGAGGAGAAGG, | Enamel-renal syndrome |
| | c.590 − 2A > G | TAATCYGCAAAGGAGGAGAAGGG | |
| 5030809 | NM_000551.3(VHL): | CCCYACCCAACGCTGCCGCCTGG | Von Hippel-Lindau syndrome, |
| | c.292T > C (p.Tyr98His) | | Hereditary cancer-predisposing |
| | | | syndrome |
| 199476132 | m.5728T > C | CAATCYACTTCTCCCGCCGCCGG, | Cytochrome-c oxidase |
| | | AATCYACTTCTCCCGCCGCCGGG | deficiency, Mitochondrial |
| | | | complex I deficiency |
| 62637012 | NM_014336.4(AIPL1): | CTGCCAGYGCCTGCTGAAGAAGG, | Leber congenital amaurosis 4 |
| | c.715T > C (p.Cys239Arg) | CCAGYGCCTGCTGAAGAAGGAGG | |
| 199476199 | NM_207352.3(CYP4V2): | AAACTGGYCCTTATACCTGTTGG, | Bietti crystalline corneoretinal |
| | c.1021T > C (p.Ser341Pro) | AACTGGYCCTTATACCTGTTGGG | dystrophy |
| 587777183 | NM_006702.4(PNPLA6): | CCTYTAACCGCAGCATCCATCGG | Boucher Neuhauser syndrome |
| | c.3053T > C (p.Phe1018Ser) | | |
| 199476389 | NM_000487.5(ARSA): | GGTCTCTYGCGGTGTGGAAAGGG | Metachromatic leukodystrophy |
| | c.899T > C (p.Leu300Ser) | | |
| 199476398 | NM_016599.4(MYOZ2): | TTAYCCCATCTCAGTAACCGTGG | Familial hypertrophic |
| | c.142T > C (p.Ser48Pro) | | cardiomyopathy 16 |
| 119456967 | NM_001037633.1(SIL1): | TTGCYGAAGGAGCTGAGATGAGG | Marinesco-Sj\xc3\xb6gren |
| | c.1370T > C (p.Leu457Pro) | | syndrome |
| 730882253 | NM_006888.4(CALM1): | GGCAYTCCGAGTCTTTGACAAGG | Long QT syndrome 14 |
| | c.268T > C (p.Phe90Leu) | | |
| 587777283 | NM_012338.3(TSPAN12): | TAATCCAYAATTTGTCATCCTGG | Exudative vitreoretinopathy 5 |
| | c.413A > G (p.Tyr138Cys) | | |
| 587777306 | NM_015884.3(MBTPS2): | GCTYTGCTTTGGATGGACAATGG | Palmoplantar keratoderma, |
| | c.1391T > C (p.Phe464Ser) | | mutilating, with periorificial |
| | | | keratotic plaques, X-linked |
| 56378716 | NM_000250.1(MPO): | TCACTCAYGTTCATGCAATGGGG | Myeloperoxidase deficiency |
| | c.752T > C (p.Met251Thr) | | |
| 587777390 | NM_005026.3(PIK3CD): | GCAGGACYGCCCCATTGCCTGGG | Activated PI3K-delta |
| | c.1246T > C (p.Cys416Arg) | | syndrome |
| 587777480 | NM_003108.3(SOX11): | TATGGYCCAAGATCGAACGCAGG | Mental retardation, autosomal |
| | c.178T > C (p.Ser60Pro) | | dominant 27 |
| 587777663 | NM_001288767.1(ARMC5): | GCCCGACYGCGGGATGCTGGTGG | Acth-independent |
| | c.1379T > C (p.Leu460Pro) | | macronodular adrenal |
| | | | hyperplasia 2 |
| 61753033 | NM_000350.2(ABCA4): | AAGGCYACATGAACTAACCAAGG | Stargardt disease, Stargardt |
| | c.5819T > C (p.Leu1940Pro) | | disease 1, Cone-rod dystrophy |
| | | | 3 |
| 200488568 | NM_002972.3(SBF1): | CAGGCGYCCTCTTGCTCAGCCGG | Charcot-Marie-Tooth disease, |
| | c.4768A > G (p.Thr1590Ala) | | type 4B3 |
| 132630274 | NM_000377.2(WAS): | CGGAGTCYGTTCTCCAGGGCAGG | Severe congenital neutropenia |
| | c.809T > C (p.Leu270Pro) | | X-linked |
| 132630308 | NM_001399.4(EDA): | CTGCYACCTAGAGTTGCGCTCGG | Hypohidrotic X-linked |
| | c.181T > C (p.Tyr61His) | | ectodermal dysplasia |
| 60934003 | NM_170707.3(LMNA): | ACGGCTCYCATCAACTCCACTGG, | Benign scapuloperoneal |
| | c.1589T > C (p.Leu530Pro) | CGGCTCYCATCAACTCCACTGGG, | muscular dystrophy with |
| | | GGCTCYCATCAACTCCACTGGGG | cardiomyopathy |
| 180177160 | NM_000030.2(AGXT): | GGTGCYGCGGATCGGCCTGCTGG, | Primary hyperoxaluria, type I |
| | c.1076T > C (p.Leu359Pro) | GTGCYGCGGATCGGCCTGCTGGG | |
| 180177222 | NM_000030.2(AGXT): | GTGCYGCTGTTCTTAACCCACGG, | Primary hyperoxaluria, type I |
| | c.449T > C (p.Leu150Pro) | TGCYGCTGTTCTTAACCCACGGG | |
| 180177254 | NM_000030.2(AGXT): | GCTCATCYCCTTCAGTGACAAGG | Primary hyperoxaluria, type I |
| | c.661T > C (p.Ser221Pro) | | |
| 180177264 | NM_000030.2(AGXT): | GGGGCYGTGACGACCAGCCCAGG | Primary hyperoxaluria, type I |
| | c.757T > C (p.Cys253Arg) | | |
| 180177293 | NM_000030.2(AGXT): | GTATCYGCATGGGCGCCTGCAGG | Primary hyperoxaluria, type I |
| | c.893T > C (p.Leu298Pro) | | |
| 376785840 | NM_001282227.1(CECR1): | GAAATCAYAGGACAAGCCTTTGG | Polyarteritis nodosa |
| | c.1232A > G (p.Tyr411Cys) | | |
| 587779393 | NM_000257.3(MYH7): | GAGCCYCCAGAGCTTGTTGAAGG | Myopathy, distal, 1 |
| | c.4937T > C (p.Leu1646Pro) | | |
| 587779410 | NM_012434.4(SLC17A5): | ATTGTACYCAGAGCACTAGAAGG | Sialic acid storage disease, |
| | c.500T > C (p.Leu167Pro) | | severe infantile type |
| 587779513 | NM_000090.3(COL3A1): | AGGYAACCCTTAATACTACCTGG | Ehlers-Danlos syndrome, type |
| | c.2337 + 2T > C | | 4 |
| | (p.Gly762_Lys779del) | | |
| 777539013 | NM_020376.3(PNPLA2): | GAACGGYGCGCGGACCCGGGCGG, | Neutral lipid storage disease |
| | c.757 + 2T > C | AACGGYGCGCGGACCCGGGCGGG | with myopathy |
| 34557412 | NM_012452.2(TNFRSF13B): | ACTTCYGTGAGAACAAGCTCAGG | Immunoglobulin A deficiency |
| | c.310T > C (p.Cys104Arg) | | 2, Common variable |
| | | | immunodeficiency 2 |
| 796052970 | NM_001165963.1(SCN1A): | CAAGCTYTGATACCTTCAGTTGG, | not provided |
| | c.1094T > C (p.Phe365Ser) | AAGCTYTGATACCTTCAGTTGGG | |
| 724159989 | NC_012920.1:m.7505T > C | CCTCCAYGACTTTTTCAAAAAGG | Deafness, nonsyndromic |
| | | | sensorineural, mitochondrial |
| 796053222 | NM_014191.3(SCN8A): | CGTCYGATCAAAGGCGCCAAAGG, | not provided |
| | c.4889T > C (p.Leu1630Pro) | GTCYGATCAAAGGCGCCAAAGGG | |
| 118192127 | NM_000540.2(RYR1): | TACTACCYGGACCAGGTGGGTGG, | Central core disease |
| | c.10817T > C | ACTACCYGGACCAGGTGGGTGGG, | |
| | (p.Leu3606Pro) | CTACCYGGACCAGGTGGGTGGGG | |
| 118192170 | NM_000540.2(RYR1): | AGGCAYTGGGGACGAGATCGAGG | Malignant hyperthermia |
| | c.14693T > C | | susceptibility type 1, Central |
| | (p.Ile4898Thr) | | core disease |
| 121917703 | NM_005247.2(FGF3): | GTACGTGYCTGTGAACGGCAAGG, | Deafness with labyrinthine |
| | c.466T > C (p.Ser156Pro) | TACGTGYCTGTGAACGGCAAGGG | aplasia microtia and |
| | | | microdontia (LAMM) |
| 690016549 | NM_005211.3(CSF1R): | CCGCCYGCCTGTGAAGTGGATGG | Hereditary diffuse |
| | c.2450T > C (p.Leu817Pro) | | leukoencephalopathy with |
| | | | spheroids |
| 690016552 | NM_005211.3(CSF1R): | GAATCCCYACCCTGGCATCCTGG | Hereditary diffuse |
| | c.2566T > C (p.Tyr856His) | | leukoencephalopathy with |
| | | | spheroids |
| 121917738 | NM_001098668.2(SFTPA2): | GGAGACTYCCGCTACTCAGATGG, | Idiopathic fibrosing alveolitis, |
| | c.593T > C (p.Phe198Ser) | GAGACTYCCGCTACTCAGATGGG | chronic form |
| 690016559 | NM_005211.3(CSF1R): | AGCCYGTACCCATGGAGGTAAGG, | Hereditary diffuse |
| | c.1957T > C (p.Cys653Arg) | GCCYGTACCCATGGAGGTAAGGG | leukoencephalopathy with |
| | | | spheroids |
| 690016560 | NM_005211.3(CSF1R): | GCAGAYCTGCTCCTTCCTTCAGG | Hereditary diffuse |
| | c.2717T > C (p.Ile906Thr) | | leukoencephalopathy with |
| | | | spheroids |
| 121917769 | NM_003361.3(UMOD): | GGCCACAYGTGTCAATGTGGTGG, | Familial juvenile gout |
| | c.376T > C (p.Cys126Arg) | GCCACAYGTGTCAATGTGGTGGG | |
| 121917773 | NM_003361.3(UMOD): | ATGGCACYGCCAGTGCAAACAGG | Glomerulocystic kidney |
| | c.943T > C (p.Cys315Arg) | | disease with hyperuricemia |
| | | | and isosthenuria |
| 121917818 | NM_007255.2(B4GALT7): | TGCYCTCCAAGCAGCACTACCGG | Ehlers-Danlos syndrome |
| | c.617T > C (p.Leu206Pro) | | progeroid type |
| 121917824 | NM_021615.4(CHST6): | GGACCYGGCGCGGGAGCCGCTGG | Macular corneal dystrophy |
| | c.827T > C (p.Leu276Pro) | | Type I |
| 121917848 | NM_000452.2(SLC10A2): | TTTCYTCTGGCTAGAATTGCTGG | Bile acid malabsorption, |
| | c.728T > C (p.Leu243Pro) | | primary |
| 121918006 | NM_000478.4(ALPL): | TGGACYATGGTGAGACCTCCAGG | Infantile hypophosphatasia |
| | c.1306T > C (p.Tyr436His) | | |
| 121918010 | NM_000478.4(ALPL): | CAAAGGCYTCTTCTTGCTGGTGG, | Infantile hypophosphatasia |
| | c.979T > C (p.Phe327Leu) | GGCYTCTTCTTGCTGGTGGAAGG | |
| 121918088 | NM_000371.3(TTR): | CCCCYACTCCTATTCCACCACGG | |
| | c.400T > C (p.Tyr134His) | | |
| 121918110 | NM_001042465.1(PSAP): | GAAGCYGCCGAAGTCCCTGTCGG | Gaucher disease, atypical, due |
| | c.1055T > C (p.Leu352Pro) | | to saposin C deficiency |
| 121918137 | NM_003730.4(RNASET2): | CCAGYGCCTTCCACCAAGCCAGG | Leukoencephalopathy, cystic, |
| | c.550T > C (p.Cys184Arg) | | without megalencephaly |
| 121918191 | NM_001127628.1(FBP1): | GGAGTYCATTTTGGTGGACAAGG | Fructose-biphosphatase |
| | c.581T > C (p.Phe194Ser) | | deficiency |
| 121918306 | NM_006946.2(SPTBN2): | ACCAAGCYGCTGGATCCCGAAGG, | Spinocerebellar ataxia 5 |
| | c.758T > C (p.Leu253Pro) | AAGCYGCTGGATCCCGAAGGTGG, | |
| | | AGCYGCTGGATCCCGAAGGTGGG | |
| 121918505 | NM_000141.4(FGFR2): | AATGCCYCCACAGTGGTCGGAGG | Pfeiffer syndrome, Neoplasm |
| | c.799T > C (p.Ser267Pro) | | of stomach |
| 121918643 | NM_003126.2(SPTA1): | GTGGAGCYGGTAGCTAAAGAAGG, | Hereditary pyropoikilocytosis, |
| | c.620T > C (p.Leu207Pro) | TGGAGCYGGTAGCTAAAGAAGGG | Elliptocytosis 2 |
| 121918646 | NM_001024858.2(SPTB): | CTCCAGCYGGAAGGATGGCTTGG | Spherocytosis type 2 |
| | c.604T > C (p.Trp202Arg) | | |
| 121918648 | NM_001024858.2(SPTB): | ATGCCYCTGTGGCTGAGGCGTGG | |
| | c.6055T > C (p.Ser2019Pro) | | |
| 727504166 | NM_000543.4(SMPD1): | TGAGGCCYGTGGCCTGCTCCTGG, | Niemann-Pick disease, type A, |
| | c.475T > C (p.Cys159Arg) | GAGGCCYGTGGCCTGCTCCTGGG | Niemann-Pick disease, type B |
| 193922915 | NM_000434.3(NEU1): | CAGCYATGGCCAGGCCCCAGTGG | Sialidosis, type II |
| | c.1088T > C (p.Leu363Pro) | | |
| 727504419 | NM_000501.3(ELN): | CAGGYAACATCTGTCCCAGCAGG, | Supravalvar aortic stenosis |
| | c.889 + 2T > C | AGGYAACATCTGTCCCAGCAGGG | |
| 376395543 | NM_000256.3(MYBPC3): | GAGACYGAAGGGCCAGGTGGAGG | Primary familial hypertrophic |
| | c.26 − 2A > G | | cardiomyopathy, Familial |
| | | | hypertrophic cardiomyopathy |
| | | | 4, Cardiomyopathy |
| 1169305 | NM_000545.6(HNF1A): | GATGCYGGCAGGGTCCTGGCTGG, | Maturity-onset diabetes of the |
| | c.1720G > A (p.Gly574Ser) | ATGCYGGCAGGGTCCTGGCTGGG, | young, type 3 |
| | | TGCYGGCAGGGTCCTGGCTGGGG | |
| 730880130 | NM_000527.4(LDLR): | CTACYGGACCGACTCTGTCCTGG, | Familial hypercholesterolemia |
| | c.1468T > C (p.Trp490Arg) | TACYGGACCGACTCTGTCCTGGG | |
| 281860286 | NM_018713.2(SLC30A10): | GGCGCTTYCGGGGGGCCTCAGGG | Hypermanganesemia with |
| | c.500T > C (p.Phe167Ser) | | dystonia, polycythemia and |
| | | | cirrhosis |
| 730880306 | NM_145693.2(LPIN1): | AAGGYACCGCGGGCCTCGCGCGG, | Myoglobinuria, acute |
| | c.1441 + 2T > C | AGGYACCGCGGGCCTCGCGCGGG | recurrent, autosomal recessive |
| 74315452 | NM_000454.4(SOD1): | TTGCAYCATTGGCCGCACACTGG | Amyotrophic lateral sclerosis |
| | c.338T > C (p.Ile113Thr) | | type 1 |
| 730880455 | NM_000169.2(GLA): | CGCGCYTGCGCTTCGCTTCCTGG | not provided |
| | c.41T > C (p.Leu14Pro) | | |
| 267606656 | NM_054027.4(ANKH): | AGCTCYGTTTCGTGATGTTTTGG | Craniometaphyseal dysplasia, |
| | c.1015T > C (p.Cys339Arg) | | autosomal dominant |
| 267606687 | NM_033409.3(SLC52A3): | AGTTACGYCAAGGTGATGCTGGG | Brown-Vialetto-Van laere |
| | c.1238T > C (p.Val413Ala) | | syndrome |
| 267606721 | NM_001928.2(CFD): | GGTGYGCGGGGGCGTGCTCGAGG, | Complement factor d |
| | c.640T > C (p.Cys214Arg) | GTGYGCGGGGGCGTGCTCGAGGG | deficiency |
| 267606747 | NM_001849.3(COL6A2): | CGCCYGCGACAAGCCACAGCAGG | Ullrich congenital muscular |
| | c.2329T > C (p.Cys777Arg) | | dystrophy |
| 431905515 | NM_001044.4(SLC6A3): | CTGCACCYCCACCAGAGCCATGG | Infantile Parkinsonism- |
| | c.671T > C (p.Leu224Pro) | | dystonia |
| 267606857 | NM_000180.3(GUCY2D): | AGAGAYCGCCAACATGTCACTGG | Cone-rod dystrophy 6 |
| | c.2846T > C (p.Ile949Thr) | | |
| 267606880 | NM_022489.3(INF2): | GCTGCYCCAGATGCCCTCTGTGG | Focal segmental |
| | c.125T > C (p.Leu42Pro) | | glomerulosclerosis 5 |
| 515726191 | NM_015713.4(RRM2B): | AACTCCTYCTACAGCAGCAAAGG | RRM2B-related mitochondrial |
| | c.581A > G (p.Glu194Gly) | | disease |
| 267606917 | NM_004646.3(NPHS1): | GCTGCCGYGCGTGGCCCGAGGGG, | Finnish congenital nephrotic |
| | c.793T > C (p.Cys265Arg) | CTGCCGYGCGTGGCCCGAGGGGG | syndrome |
| 267607104 | NM_001199107.1(TBC1D24): | CAAGTTCYTCCACAAGGTGAGGG, | Myoclonic epilepsy, familial |
| | c.751T > C (p.Phe251Leu) | TTCYTCCACAAGGTGAGGGCCGG | infantile |
| 267607182 | NM_144631.5(ZNF513): | TGGGCGCYGCATGCGAGGAGAGG, | Retinitis pigmentosa 58 |
| | c.1015T > C (p.Cys339Arg) | CGCYGCATGCGAGGAGAGGCTGG | |
| 267607211 | NM_000229.1(LCAT): | TATGACYGGCGGCTGGAGCCCGG | Norum disease |
| | c.508T > C (p.Trp170Arg) | | |
| 267607215 | NM_016269.4(LEF1): | GAACGAGYCTGAAATCATCCCGG | Sebaceous tumors, somatic |
| | c.181T > C (p.Ser61Pro) | | |
| 587783580 | NM_178151.2(DCX): | AAAAAACYCTACACTCTGGATGG | Heterotopia |
| | c.683T > C (p.Leu228Pro) | | |
| 587783644 | NM_004004.5(GJB2): | GATCCYCGTTGTGGCTGCAAAGG | Hearing impairment |
| | c.107T > C (p.Leu36Pro) | | |
| 587783653 | NM_005682.6(ADGRG1): | CCCTGCYCACCTGCCTTTCCTGG | Polymicrogyria, bilateral |
| | c.1460T > C (p.Leu487Pro) | | frontoparietal |
| 587783863 | NM_000252.2(MTM1): | GGAAYCTTTAAAAAAAGTGAAGG | Severe X-linked myotubular |
| | c.958T > C (p.Ser320Pro) | | myopathy |
| 267607751 | NM_000249.3(MLH1): | ATCACGGYAAGAATGGTACATGG, | Hereditary Nonpolyposis |
| | c.453 + 2T > C | TCACGGYAAGAATGGTACATGGG | Colorectal Neoplasms |
| 119103227 | NM_000411.6(HLCS): | CTATCYTTCTCAGGGAGGGAAGG | Holocarboxylase synthetase |
| | c.710T > C (p.Leu237Pro) | | deficiency |
| 119103237 | NM_005787.5(ALG3): | GATTGACYGGAAGGCCTACATGG | Congenital disorder of |
| | c.211T > C (p.Trp71Arg) | | glycosylation type 1D |
| 398122806 | NM_003172.3(SURF1): | CCACYGGCATTATCGAGACCTGG | Congenital myasthenic |
| | c.679T > C (p.Trp227Arg) | | syndrome, acetazolamide- |
| | | | responsive |
| 80338747 | NM_004525.2(LRP2): | GTACCTGYACTGGGCTGACTGGG | Donnai Barrow syndrome |
| | c.7564T > C (p.Tyr2522His) | | |
| 398122838 | NM_001271723.1(FBXO38): | TTCCTYGTATCCCAATGCTAAGG | Distal hereditary motor |
| | c.616T > C (p.Cys206Arg) | | neuronopathy 2D |
| 398122989 | NM_014495.3(ANGPTL3): | ACAAAACYTCAATGAAACGTGGG | Hypobetalipoproteinemia, |
| | c.883T > C (p.Phe295Leu) | | familial, 2 |
| 80338945 | NM_004004.5(GJB2): | GCTCCYAGTGGCCATGCACGTGG | Deafness, autosomal recessive |
| | c.269T > C (p.Leu90Pro) | | 1A, Hearing impairment |
| 80338956 | NM_000334.4(SCN4A): | AAGATCAYTGGCAATTCAGTGGG, | Hyperkalemic Periodic |
| | c.2078T > C (p.Ile693Thr) | AGATCAYTGGCAATTCAGTGGGG, | Paralysis Type 1, |
| | | GATCAYTGGCAATTCAGTGGGGG | Paramyotonia congenita of von |
| | | | Eulenburg |
| 267608131 | NM_000179.2(MSH6): | CGGYAACTAACTAACTATAATGG | Hereditary Nonpolyposis |
| | c.4001 + 2T > C | | Colorectal Neoplasms |
| 587784573 | NM_004963.3(GUCY2C): | TCCCYGTGCTGCTGGAGTTGTGG, | Meconium ileus |
| | c.2782T > C (p.Cys928Arg) | CCCYGTGCTGCTGGAGTTGTGGG | |
| 267608511 | NM_003159.2(CDKL5): | CCAACYTTTTACTATTCAGAAGG | Early infantile epileptic |
| | c.659T > C (p.Leu220Pro) | | encephalopathy 2 |
| 373842615 | NM_000118.3(ENG): | CCGCCYGCGGGGATAAAGCCAGG, | Haemorrhagic telangiectasia 1 |
| | c.1273 − 2A > G | CGCCYGCGGGGATAAAGCCAGGG | |
| 185492581 | NM_000335.4(SCN5A): | GAATCTYCACAGCCGCTCTCCGG | Brugada syndrome |
| | c.376A > G (p.Lys126Glu) | | |
| 200533370 | NM_133499.2(SYN1): | GATGYCTGACGGGTAGCCTGTGG, | Epilepsy, X-linked, with |
| | c.1699A > G (p.Thr567Ala) | ATGYCTGACGGGTAGCCTGTGGG | variable learning disabilities |
| | | | and behavior disorders, not |
| | | | specified |
| 118203981 | NM_148960.2(CLDN19): | GCTCCYGGGCTTCGTGGCCATGG | Hypomagnesemia 5, renal, |
| | c.269T > C (p.Leu90Pro) | | with ocular involvement |
| 137853892 | NM_001235.3(SERPINH1): | GTCGCYAGGGCTCGTGTCGCTGG, | Osteogenesis imperfecta type |
| | c.233T > C (p.Leu78Pro) | TCGCYAGGGCTCGTGTCGCTGGG | 10 |
| 118204024 | NM_000263.3(NAGLU): | GGCCGACYTCTCCGTGTCGGTGG | Mucopolysaccharidosis, MPS- |
| | c.142T > C (p.Phe48Leu) | | III-B |
| 690016563 | NM_005211.3(CSF1R): | CAACCYGCAGTTTGGTGAGATGG | Hereditary diffuse |
| | c.1745T > C (p.Leu582Pro) | | leukoencephalopathy with |
| | | | spheroids |
| 58380626 | NM_000526.4(KRT14): | CGCCACCYACCGCCGCCTGCTGG, | Epidermolysis bullosa |
| | c.1243T > C (p.Tyr415His) | CACCYACCGCCGCCTGCTGGAGG, | herpetiformis, Dowling-Meara |
| | | ACCYACCGCCGCCTGCTGGAGGG | |
| 113994151 | NM_207346.2(TSEN54): | TTGAAGYCTCCCGCGGTGAGCGG, | Pontocerebellar hypoplasia |
| | c.277T > C (p.Ser93Pro) | AAGYCTCCCGCGGTGAGCGGCGG | type 4 |
| 113994206 | NM_004937.2(CTNS): | TGGTCYGAGCTTCGACTTCGTGG | Cystinosis |
| | c.473T > C (p.Leu158Pro) | | |
| 62516109 | NM_000277.1(PAH): | CCACTTCYTGAAAAGTACTGTGG | Phenylketonuria |
| | c.638T > C (p.Leu213Pro) | | |
| 370011798 | NM_001302946.1(TRNT1): | GCAAYTGCAGAAAATGCAAAAGG | Sideroblastic anemia with B- |
| | c.668T > C (p.Ile223Thr) | | cell immunodeficiency, |
| | | | periodic fevers, and |
| | | | developmental delay |
| 62517167 | NM_000277.1(PAH): | AAGATCTYGAGGCATGACATTGG | Mild non-PKU |
| | c.293T > C (p.Leu98Ser) | | hyperphenylalanemia |
| 12021720 | NM_001918.3(DBT): | GACYCACAGAGCCCAATTTCTGG | Intermediate maple syrup urine |
| | c.1150G > A (p.Gly384Ser) | | disease type 2 |
| 104886289 | NM_000495.4(COL4A5): | TCCCCATYGTCCTCAGGGATGGG | Alport syndrome, X-linked |
| | c.4756T > C (p.Cys1586Arg) | | recessive |
| 370471013 | NC_012920.1:m.5559A > G | CAACYTACTGAGGGCTTTGAAGG | Leigh disease |
| 121434215 | NM_000487.5(ARSA): | GCCTTCCYGCCCCCCCATCAGGG | Metachromatic |
| | c.410T > C (p.Leu137Pro) | | leukodystrophy, adult type |
| 386134128 | NM_000096.3(CP): | ACACTACYACATTGCCGCTGAGG | Deficiency of ferroxidase |
| | c.1123T > C (p.Tyr375His) | | |
| 121434275 | NM_001127328.2(ACADM): | GTGCAGAYACTTGGAGGCAATGG | Medium-chain acyl-coenzyme |
| | c.1136T > C (p.Ile379Thr) | | A dehydrogenase deficiency |
| 121434276 | NM_001127328.2(ACADM): | CAGCGAYGTTCAGATACTAGAGG | Medium-chain acyl-coenzyme |
| | c.742T > C (p.Cys248Arg) | | A dehydrogenase deficiency |
| 121434284 | NM_002225.3(IVD): | ATGGGCYAAGCGAGGAGCAGAGG | ISOVALERIC ACIDEMIA, |
| | c.134T > C (p.Leu45Pro) | | TYPE I |
| 121434334 | NM_005908.3(MANBA): | ATTACGYCCAGTCCTACAAATGG, | Beta-D-mannosidosis |
| | c.1513T > C (p.Ser505Pro) | TTACGYCCAGTCCTACAAATGGG, | |
| | | TACGYCCAGTCCTACAAATGGGG | |
| 121434366 | NM_000159.3(GCDH): | CGCCCGGYACGGCATCGCGTGGG, | Glutaric aciduria, type 1 |
| | c.883T > C (p.Tyr295His) | GCCCGGYACGGCATCGCGTGGGG | |
| 60715293 | NM_000424.3(KRT5): | GTTTGCCYCCTTCATCGACAAGG | Epidermolysis bullosa |
| | c.541T > C (p.Ser181Pro) | | herpetiformis, Dowling-Meara |
| 121434409 | NM_001003722.1(GLE1): | AAGGACAYTCCTGTCCCCAAGGG | Lethal arthrogryposis with |
| | c.2051T > C (p.Ile684Thr) | | anterior horn cell disease |
| 121434434 | NM_001287.5(CLCN7): | GGGCCYGCGGCACCTGGTGGTGG | Osteopetrosis autosomal |
| | c.2297T > C (p.Leu766Pro) | | recessive 4 |
| 121434455 | NM_000466.2(PEX1): | GATGACCYTGACCTCATTGCTGG | Zellweger syndrome |
| | c.1991T > C (p.Leu664Pro) | | |
| 199422317 | NM_001099274.1(TINF2): | CTGYTTCCCTTTAGGAATCTCGG | Aplastic anemia |
| | c.862T > C (p.Phe288Leu) | | |
| 104895221 | NM_001065.3(TNFRSF1A): | CTCTTCTYGCACAGTGGACCGGG | TNF receptor-associated |
| | c.349T > C (p.Cys117Arg) | | periodic fever syndrome |
| | | | (TRAPS) |
| 137854459 | NM_000138.4(FBN1): | GGGACAYGTTACAACACCGTTGG | Marfan syndrome |
| | c.4987T > C (p.Cys1663Arg) | | |
| 387907075 | NM_024027.4(COLEC11): | CAGCTGYCCTGCCAGGGCCGCGG, | Carnevale syndrome |
| | c.505T > C (p.Ser169Pro) | AGCTGYCCTGCCAGGGCCGCGGG, | |
| | | GCTGYCCTGCCAGGGCCGCGGGG, | |
| | | CTGYCCTGCCAGGGCCGCGGGGG | |
| 1048095 | NM_000352.4(ABCC8): | TGCYGTCCAAAGGCACCTACTGG | Permanent neonatal diabetes |
| | c.674T > C (p.Leu225Pro) | | mellitus |
| 796065347 | NM_019074.3(DLL4): | GAAYGTCCCCCCAACTTCACCGG | Adams-Oliver syndrome, |
| | c.1168T > C (p.Cys390Arg) | | ADAMS-OLIVER |
| | | | SYNDROME 6 |
| 137852347 | NM_000402.4(G6PD): | AGGGYACCTGGACGACCCCACGG | Anemia, nonspherocytic |
| | c.1054T > C (p.Tyr352His) | | hemolytic, due to G6PD |
| | | | deficiency |
| 74315327 | NM_213653.3(HFE2): | GGACCYCGCCTTCCATTCGGCGG | Hemochromatosis type 2A |
| | c.302T > C (p.Leu101Pro) | | |
| 137852579 | NM_000044.3(AR): | GTCCYGGAAGCCATTGAGCCAGG | |
| | c.2033T > C (p.Leu678Pro) | | |
| 137852636 | NM_001166107.1(HMGCS2): | CCCTCYTCAATGCTGCCAACTGG | mitochondrial 3-hydroxy-3- |
| | c.520T > C (p.Phe174Leu) | | methylglutaryl-CoA synthase |
| | | | deficiency |
| 137852661 | NM_033163.3(FGF8): | TTCCCTGYTCCGGGCTGGCCGGG | Kallmann syndrome 6 |
| | c.118T > C (p.Phe40Leu) | | |
| 121912967 | NM_005215.3(DCC): | AGCCCAYGCCAACAATCCACTGG | |
| | c.503T > C (p.Met168Thr) | | |
| 137852806 | NM_001039523.2(CHRNA1): | TGTGYTCCTTCTGGTCATCGTGG | Myasthenic syndrome, |
| | c.901T > C (p.Phe301Leu) | | congenital, fast-channel |
| 137852850 | NM_182760.3(SUMF1): | GGCGACYCCTTTGTCTTTGAAGG | Multiple sulfatase deficiency |
| | c.463T > C (p.Ser155Pro) | | |
| 137852886 | NM_000158.3(GBE1): | AATGTACYACCAAGAATCAAAGG | Glycogen storage disease, type |
| | c.671T > C (p.Leu224Pro) | | IV, GLYCOGEN STORAGE |
| | | | DISEASE IV, |
| | | | NONPROGRESSIVE |
| | | | HEPATIC |
| 137852911 | NM_000419.3(ITGA2B): | CTGGTGCYTGGGGCTCCTGGCGG | Glanzmann thrombasthenia |
| | c.641T > C (p.Leu214Pro) | | |
| 137852948 | NM_138694.3(PKHD1): | GAGCCCAYTGAAATACGCTCAGG | Polycystic kidney disease, |
| | c.10658T > C | | infantile type |
| | (p.Ile3553Thr) | | |
| 137852964 | NM_024960.4(PANK2): | ATTGACYCAGTCGGATTCAATGG | |
| | c.178T > C (p.Ser60Pro) | | |
| 137853020 | NM_006899.3(IDH3B): | TGCGGCYGAGGTAGGTGGTCTGG, | Retinitis pigmentosa 46 |
| | c.395T > C (p.Leu132Pro) | GCGGCYGAGGTAGGTGGTCTGGG | |
| 137853249 | NM_033500.2(HK1): | GACTTCTYGGCCCTGGATCTTGG, | Hemolytic anemia due to |
| | c.1550T > C (p.Leu517Ser) | TTCTYGGCCCTGGATCTTGGAGG | hexokinase deficiency |
| 137853270 | NM_000444.5(PHEX): | AGCYCCAGAAGCCTTTCTTTTGG | Familial X-linked |
| | c.1664T > C (p.Leu555Pro) | | hypophosphatemic vitamin D |
| | | | refractory rickets |
| 137853325 | NM_003639.4(IKBKG): | TGGAGYGCATTGAGTAGGGCCGG | Hypohidrotic ectodermal |
| | c.1249T > C (p.Cys417Arg) | | dysplasia with immune |
| | | | deficiency, Hyper-IgM |
| | | | immunodeficiency, X-linked, |
| | | | with hypohidrotic ectodermal |
| | | | dysplasia |
| 28932769 | NM_002055.4(GFAP): | GGACCYGCTCAATGTCAAGCTGG | Alexander disease |
| | c.1055T > C (p.Leu352Pro) | | |
| 397507439 | NM_002769.4(PRSS1): | TACCAGGYGTCCCTGAATTCTGG | Hereditary pancreatitis |
| | c.116T > C (p. Val39Ala) | | |
| 387906446 | NM_000132.3(F8): | AAAGAAYCTGTAGATCAAAGAGG | Hereditary factor VIII |
| | c.1729T > C (p.Ser577Pro) | | deficiency disease |
| 387906482 | NM_000133.3(F9): | ACGAACAYCTTCCTCAAATTTGG | Hereditary factor IX deficiency |
| | c.1031T > C (p.Ile344Thr) | | disease |
| 387906508 | NM_000131.4(F7): | GACGTYCTCTGAGAGGACGCTGG | Factor VII deficiency |
| | c.983T > C (p.Phe328Ser) | | |
| 387906532 | NM_001040113.1(MYH11): | GAAGCYGGAGGCGCAGGTGCAGG | Aortic aneurysm, familial |
| | c.3791T > C (p.Leu1264Pro) | | thoracic 4 |
| 387906658 | NM_002465.3(MYBPC1): | CAAACCYATATCCGCAGAGTTGG | Distal arthrogryposis type 1B |
| | c.2566T > C (p.Tyr856His) | | |
| 387906701 | NM_003491.3(NAA10): | TGGCCTTYCCTGGCCCCAGGTGG, | N-terminal acetyltransferase |
| | c.109T > C (p.Ser37Pro) | GGCCTTYCCTGGCCCCAGGTGGG | deficiency |
| 387906717 | NM_000377.2(WAS): | GACTTCAYTGAGGACCAGGGTGG, | Severe congenital neutropenia |
| | c.881T > C (p.Ile294Thr) | ACTTCAYTGAGGACCAGGGTGGG | X-linked |
| 387906809 | NM_000287.3(PEX6): | CTTCYGGGCCGGGACCGTGATGG, | Peroxisome biogenesis |
| | c.1601T > C (p.Leu534Pro) | TTCYGGGCCGGGACCGTGATGGG | disorder 4B |
| 387906965 | NM_024513.3(FYCO1): | CAGCCYGATCCCCATCACTGTGG | Cataract, autosomal recessive |
| | c.4127T > C (p.Leu1376Pro) | | congenital 2 |
| 387906967 | NM_006147.3(IRF6): | GCCYCTACCCTGGGCTCATCTGG | Van der Woude syndrome, |
| | c.65T > C (p.Leu22Pro) | | Popliteal pterygium syndrome |
| 387906982 | NM_025132.3(WDR19): | TCTCACYGCTAGAAAAGACTTGG | Asphyxiating thoracic |
| | c.20T > C (p.Leu7Pro) | | dystrophy 5 |
| 387907072 | NM_032446.2(MEGF10): | GGGCAGYGTACTTGCCGCACTGG | Myopathy, areflexia, |
| | c.2320T > C (p.Cys774Arg) | | respiratory distress, and |
| | | | dysphagia, early-onset, |
| | | | Myopathy, areflexia, |
| | | | respiratory distress, and |
| | | | dysphagia, early-onset, mild |
| | | | variant |
| 137854499 | NM_005502.3(ABCA1): | GAGTYCTTTGCCCTTTTGAGAGG | Familial |
| | c.6026T > C (p.Phe2009Ser) | | hypoalphalipoproteinemia |
| 387907117 | NM_000196.3(HSD11B2): | CCGCCGCYATTACCCCGGCCAGG, | Apparent mineralocorticoid |
| | c.1012T > C (p.Tyr338His) | CGCCGCYATTACCCCGGCCAGGG | excess |
| 387907170 | NM_004453.3(ETFDH): | CCAAAACYCACCTTTCCTGGTGG | |
| | c.1130T > C (p.Leu377Pro) | | |
| 387907205 | NM_033360.3(KRAS): | GGACCAGYACATGAGGACTGGGG, | Cardiofaciocutaneous |
| | c.211T > C (p.Tyr71His) | CCAGYACATGAGGACTGGGGAGG, | syndrome 2 |
| | | CAGYACATGAGGACTGGGGAGGG | |
| 387907240 | NM_024110.4(CARD14): | CAGCAGCYGCAGGAGCACCTGGG | Pityriasis rubra pilaris |
| | c.467T > C (p.Leu156Pro) | | |
| 387907282 | NM_152296.4(ATP1A3): | TGCCATCYCACTGGCGTACGAGG | Alternating hemiplegia of |
| | c.2431T > C (p.Ser811Pro) | | childhood 2 |
| 387907361 | NM_005120.2(MED12): | AGGACYCTGAGCCAGGGGCCCGG | Ohdo syndrome, X-linked |
| | c.3493T > C (p.Ser1165Pro) | | |
| 28933970 | NM_006194.3(PAX9): | GGCCGCYGCCCAACGCCATCCGG | Tooth agenesis, selective, 3 |
| | c.62T > C (p.Leu21Pro) | | |
| 137854472 | NM_000138.4(FBN1): | TGCACYTGCCGTGGGTGCAGAGG | |
| | c.3128A > G (p.Lys1043Arg) | | |
| 727504261 | NM_000257.3(MYH7): | AGCGCYCCTCAGCATCTGCCAGG | Cardiomyopathy, not specified |
| | c.2708A > G (p.Glu903Gly) | | |
| 81002853 | NM_000059.3(BRCA2): | ACCACYGGGGGTAAAAAAAGGGG, | Familial cancer of breast, |
| | c.476 − 2A > G | TACCACYGGGGGTAAAAAAAGGG, | Breast-ovarian cancer, familial |
| | | ATACCACYGGGGGTAAAAAAAGG | 2, Hereditary cancer- |
| | | | predisposing syndrome |
| 119473032 | NM_021020.3(LZTS1): | CCCTYCTCGGAGCCCTGTAGAGG | |
| | c.355A > G (p.Lys119Glu) | | |
| 193922801 | NM_000540.2(RYR1): | TTCYCCTCCACGCTCTCGCCTGG | not provided |
| | c.7043A > G (p.Glu2348Gly) | | |
| 36210419 | NM_000218.2(KCNQ1): | GCCCCTYGGAGCCCACGCAGAGG | Torsades de pointes, Cardiac |
| | c.652A > G (p.Lys218Glu) | | arrhythmia |
| 121964989 | NM_000108.4(DLD): | TTCTCYAAAAGCTTCTGATAAGG | Maple syrup urine disease, |
| | c.1483A > G (p.Arg495Gly) | | type 3 |
| 28936669 | NM_000095.2(COMP): | ATTGYCGTCGTCGTCGTCGCAGG | |
| | c.1418A > G (p.Asp473Gly) | | |
| 28936696 | NM_018488.2(TBX4): | GTACYGTAAGGAAGATTCTCGGG, | Ischiopatellar dysplasia |
| | c.1592A > G (p.Gln531Arg) | GGTACYGTAAGGAAGATTCTCGG | |
| 121965077 | NM_000137.2(FAH): | TCCYGGTCTGACCATTCCCCAGG | Tyrosinemia type I |
| | c.1141A > G (p.Arg381Gly) | | |
| 794728203 | NM_000138.4(FBN1): | ACTCAYCAATATCTGCAAAATGG | Thoracic aortic aneurysms and |
| | c.3344A > G (p.Asp1115Gly) | | aortic dissections |
| 786205436 | NM_003002.3(SDHD): | GAATAGYCCATCGCAGAGCAAGG | Fatal infantile mitochondrial |
| | c.275A > G (p.Asp92Gly) | | cardiomyopathy |
| 72551317 | NM_000784.3(CYP27A1): | AGTCCACYTGGGGAGGAAGGTGG | Cholestanol storage disease |
| | c.776A > G (p.Lys259Arg) | | |
| 786205687 | NM_016218.2(POLK): | ATTCACAYTCTTCAACTTAATGG | Malignant tumor of prostate |
| | c.1385A > G (p.Asn462Ser) | | |
| 794728280 | NM_000138.4(FBN1): | TGTTCAYACTGGAAGCCGGCGGG, | Thoracic aortic aneurysms and |
| | c.7916A > G (p.Tyr2639Cys) | CTGTTCAYACTGGAAGCCGGCGG | aortic dissections |
| 28937317 | NM_000335.4(SCN5A): | GCAYTGACCACCACCTCAAGTGG | Long QT syndrome 3, |
| | c.3971A > G (p.Asn1324Ser) | | Congenital long QT syndrome |
| 786205854 | NM_144499.2(GNAT1): | CGGAGYCCTTCCACAGCCGCTGG | NIGHT BLINDNESS, |
| | c.386A > G (p.Asp129Gly) | | CONGENITAL |
| | | | STATIONARY, TYPE 1G |
| 104893776 | NM_000539.3(RHO): | GGATGYACCTGAGGACAGGCAGG | Retinitis pigmentosa 4 |
| | c.533A > G (p.Tyr178Cys) | | |
| 28937590 | NM_001257342.1(BCS1L): | GACACYGAGGTGCTGAGTACGGG, | GRACILE syndrome |
| | c.232A > G (p.Ser78Gly) | CGACACYGAGGTGCTGAGTACGG | |
| 104893866 | NM_000320.2(QDPR): | TGCCGYACCCGATCATACCTGGG, | Dihydropteridine reductase |
| | c.449A > G (p.Tyr150Cys) | ATGCCGYACCCGATCATACCTGG | deficiency |
| 587776590 | NM_015629.3(PRPF31): | GACAYACCCCTGGGTGGTGGAGG, | Retinitis pigmentosa 11 |
| | c.527 + 3A > G | GCGGACAYACCCCTGGGTGGTGG | |
| 104894015 | NM_000162.3(GCK): | GTAGYAGCAGGAGATCATCGTGG | Hyperinsulinemic |
| | c.641A > G (p.Tyr214Cys) | | hypoglycemia familial 3 |
| 202247823 | NM_000532.4(PCCB): | ATATYTGCATGTTTTCTCCAAGG | Propionic acidemia |
| | c.1606A > G (p.Asn536Asp) | | |
| 104894199 | NM_000073.2(CD3G): | CCAYGTCAGTCTCTGTCCTCCGG | Immunodeficiency 17 |
| | c.1A > G (p.Met1Val) | | |
| 104894208 | NM_001814.4(CTSC): | CTCCYGAGGGCTTAGGATTGGGG, | Papillon-Lef\xc3\xa8vre |
| | c.857A > G (p.Gln286Arg) | CCTCCYGAGGGCTTAGGATTGGG, | syndrome, Haim-Munk |
| | | ACCTCCYGAGGGCTTAGGATTGG | syndrome |
| 104894211 | NM_001814.4(CTSC): | TCCTACAYAGTGGTACTCAGAGG | Papillon-Lef\xc3\xa8vre |
| | c.1040A > G (p.Tyr347Cys) | | syndrome, Periodontitis, |
| | | | aggressive, 1 |
| 104894290 | NM_000448.2(RAG1): | CTGYACTGGCAGAGGGATTCTGG | Histiocytic medullary |
| | c.2735A > G (p.Tyr912Cys) | | reticulosis |
| 104894354 | NM_000217.2(KCNA1): | GCGYTTCCACGATGAAGAAGGGG, | Episodic ataxia type 1 |
| | c.676A > G (p.Thr226Ala) | AGCGYTTCCACGATGAAGAAGGG, | |
| | | CAGCGYTTCCACGATGAAGAAGG | |
| 104894425 | NM_014239.3(EIF2B2): | AGTTGTCYCAATACCTGCTTTGG | Leukoencephalopathy with |
| | c.638A > G (p.Glu213Gly) | | vanishing white matter, |
| | | | Ovarioleukodystrophy |
| 104894450 | NM_000270.3(PNP): | ATAYCTCCAACCTCAAACTTGGG, | Purine-nucleoside |
| | c.383A > G (p.Asp128Gly) | GATAYCTCCAACCTCAAACTTGG | phosphorylase deficiency |
| 147394623 | NM_024887.3(DHDDS): | GGCACTYCTTGGCATAGCGACGG | Retinitis pigmentosa 59 |
| | c.124A > G (p.Lys42Glu) | | |
| 60723330 | NM_005557.3(KRT16): | GCGGTCAYTGAGGTTCTGCATGG | Pachyonychia congenita, type |
| | c.374A > G (p.Asn125Ser) | | 1, Palmoplantar keratoderma, |
| | | | nonepidermolytic, focal |
| 104894634 | NM_030665.3(RAI1): | CTGCTGCYGTCGTCGTCGCTTGG | Smith-Magenis syndrome |
| | c.4685A > G (p.Gln1562Arg) | | |
| 104894730 | NM_000363.4(TNNI3): | CCTYCTTCACCTGCTTGAGGTGG, | Familial restrictive |
| | c.532A > G (p.Lys178Glu) | CCTCCTYCTTCACCTGCTTGAGG | cardiomyopathy 1 |
| 104894816 | NM_002049.3(GATA1): | GTCCTGYCCCTCCGCCACAGTGG | GATA-1-related |
| | c.653A > G (p.Asp218Gly) | | thrombocytopenia with |
| | | | dyserythropoiesis |
| 794726773 | NM_001165963.1(SCN1A): | GTGCCAYACCTGGTGTGGGGAGG | Severe myoclonic epilepsy in |
| | c.1662 + 3A > G | | infancy |
| 104894861 | NM_000202.6(IDS): | AAAGACTYTTCCCACCGACATGG | Mucopolysaccharidosis, MPS- |
| | c.404A > G (p.Lys135Arg) | | II |
| 104894874 | NM_000266.3(NDP): | TGGYGCCTCATGCAGCGTCGAGG | |
| | c.125A > G (p.His42Arg) | | |
| 191205969 | NM_002420.5(TRPM1): | AAGCYCTTAATATCTGTGCATGG | Congenital stationary night |
| | c.296T > C (p.Leu99Pro) | | blindness, type 1C |
| 794727073 | NM_019109.4(ALG1): | TAAACYGCAGAGAGAACCAAGGG, | Congenital disorder of |
| | c.1188 − 2A > G | GTAAACYGCAGAGAGAACCAAGG | glycosylation type 1K |
| 281875236 | NM_001004334.3(GPR179): | CCCACAYATCCATCTGCCTGCGG | Congenital stationary night |
| | c.659A > G (p.Tyr220Cys) | | blindness, type 1E |
| 28939094 | NM_015915.4(ATL1): | CACCCAYCTTCTTCACCCCTCGG | Spastic paraplegia 3 |
| | c.1222A > G (p.Met408Val) | | |
| 281875324 | NM_005359.5(SMAD4): | ATCCATTYCAAAGTAAGCAATGG | Juvenile polyposis syndrome, |
| | c.989A > G (p.Glu330Gly) | | Hereditary cancer-predisposing |
| | | | syndrome |
| 77173848 | NM_000037.3(ANK1): | GGGCCYGGCCCGCACGTCACAGG | Spherocytosis, type 1, |
| | c.−108T > C | | autosomal recessive |
| 150181226 | NM_001159772.1(CANT1): | CGTCYGTACGTGGGCGGCCTGGG, | Desbuquois syndrome |
| | c.671T > C (p.Leu224Pro) | GCGTCYGTACGTGGGCGGCCTGG | |
| 397514253 | NM_000041.3(APOE): | CGCCCYGCGGCCGAGAGGGCGGG, | Familial type 3 |
| | c.237 − 2A > G | GCGCCCYGCGGCCGAGAGGGCGG | hyperlipoproteinemia |
| 397514348 | NM_000060.3(BTD): | GTTCAYAGATGTCAAGGTTCTGG | Biotinidase deficiency |
| | c.278A > G (p.Tyr93Cys) | | |
| 397514415 | NM_000060.3(BTD): | GGCAYACAGCTCTTTGGATAAGG | Biotinidase deficiency |
| | c.1313A > G (p.Tyr438Cys) | | |
| 397514501 | NM_007171.3(POMT1): | GAGCATYCTCTGTTTCAAAGAGG | Limb-girdle muscular |
| | c.430A > G (p.Asn144Asp) | | dystrophy- |
| | | | dystroglycanopathy, type C1 |
| 370382601 | NM_174917.4(ACSF3): | GGCAGCAYTGCACTGACAGGCGG | not provided |
| | c.1A > G (p.Met1Val) | | |
| 72554332 | NM_000531.5(OTC): | AAGGACTYCCCTTGCAATAAAGG | Ornithine |
| | c.238A > G (p.Lys80Glu) | | carbamoyltransferase |
| | | | deficiency |
| 397514599 | NM_033109.4(PNPT1): | GACTYCAGATGTAACTCTTATGG | Deafness, autosomal recessive |
| | c.1424A > G (p.Glu475Gly) | | 70 |
| 397514650 | NM_000108.4(DLD): | GACTCYAGCTATATCTTCACAGG | Maple syrup urine disease, |
| | c.1444A > G (p.Arg482Gly) | | type 3 |
| 397514675 | NM_003156.3(STIM1): | TTCCACAYCCACATCACCATTGG | Myopathy with tubular |
| | c.251A > G (p.Asp84Gly) | | aggregates |
| 794728378 | NM_000238.3(KCNH2): | ATCYTCTCTGAGTTGGTGTTGGG, | Cardiac arrhythmia |
| | c.1913A > G (p.Lys638Arg) | GATCYTCTCTGAGTTGGTGTTGG | |
| 397514711 | NM_002163.2(IRF8): | AACCTCGYCTTCCAAGTGGCTGG | Autosomal dominant |
| | c.238A > G (p.Thr80Ala) | | CD11C+/CD1C+ dendritic cell |
| | | | deficiency |
| 397514729 | NM_000388.3(CASR): | CCCCCTYCTTTTGGGCTCGCTGG | Hypocalcemia, autosomal |
| | c.85A > G (p.Lys29Glu) | | dominant 1, with bartter |
| | | | syndrome |
| 397514743 | NM_022114.3(PRDM16): | GCCGCCGYTTTGGCTGGCACGGG | Left ventricular |
| | c.2447A > G (p.Asn816Ser) | | noncompaction 8 |
| 397514757 | NM_005689.2(ABCB6): | TGGGCYGTTCCAAGACACCAGGG, | Dyschromatosis universalis |
| | c.508A > G (p.Ser170Gly) | GTGGGCYGTTCCAAGACACCAGG | hereditaria 3 |
| 28940313 | NM_152443.2(RDH12): | CACTGCGYAGGTGGTGACCCCGG | Leber congenital amaurosis 13 |
| | c.677A > G (p.Tyr226Cys) | | |
| 794728538 | NM_000218.2(KCNQ1): | GTCTYCTACTCGGTTCAGGCGGG, | Cardiac arrhythmia |
| | c.1787A > G (p.Glu596Gly) | TGTCTYCTACTCGGTTCAGGCGG | |
| 794728569 | NM_000218.2(KCNQ1): | AGGYCTGTGGAGTGCAGGAGAGG | Cardiac arrhythmia |
| | c.605A > G (p.Asp202Gly) | | |
| 794728573 | NM_000218.2(KCNQ1): | GCCYGCAGTGGAGAGAGGAGAGG | Cardiac arrhythmia |
| | c.1515 − 2A > G | | |
| 370874727 | NM_003494.3(DYSF): | CCGCCCYGGAGACACGAAGCTGG | Limb-girdle muscular |
| | c.33492A > G | | dystrophy, type 2B |
| 794728859 | NM_198056.2(SCN5A): | ACCYGTCGAGATAATGGGTCAGG | not provided |
| | c.2788 − 2A > G | | |
| 794728887 | NM_198056.2(SCN5A): | CCTCTGYCATGAAGATGTCCTGG | not provided |
| | c.4462A > G (p.Thr1488Ala) | | |
| 28940878 | NM_000372.4(TYR): | CTCCTGYCCCCGCTCCACGGTGG | Tyrosinase-negative |
| | c.125A > G (p.Asp42Gly) | | oculocutaneous albinism |
| 397515420 | NM_172107.2(KCNQ2): | GCAYGACACTGCAGGGGGGTGGG, | Early infantile epileptic |
| | c.1636A > G (p.Met546Val) | CGCAYGACACTGCAGGGGGGTGG, | encephalopathy 7 |
| | | AACCGCAYGACACTGCAGGGGGG | |
| 397515428 | NM_001410.2(MEGF8): | GACYCCCGTGAAATGATTCCCGG | Carpenter syndrome 2 |
| | c.7099A > G (p.Ser2367Gly) | | |
| 143601447 | NM_201631.3(TGM5): | TCAACCYCACCCTGTACTTCAGG | Peeling skin syndrome, acral |
| | c.122T > C (p.Leu41Pro) | | type |
| 397515519 | NM_000207.2(INS): | GGGCYTTATTCCATCTCTCTCGG | Permanent neonatal diabetes |
| | c.*59A > G | | mellitus |
| 397515523 | NM_000370.3(TTPA): | CAGGYCCAGATCGAAATCCCGGG, | Ataxia with vitamin E |
| | c.191A > G (p.Asp64Gly) | CCAGGYCCAGATCGAAATCCCGG | deficiency |
| 397515891 | NM_000256.3(MYBPC3): | TACTTGCYGTAGAACAGAAGGGG | Familial hypertrophic |
| | c.1224 − 2A > G | | cardiomyopathy 4, |
| | | | Cardiomyopathy |
| 397516082 | NM_000256.3(MYBPC3): | GTCCCYGTGTCCCGCAGTCTAGG | Familial hypertrophic |
| | c.927 − 2A > G | | cardiomyopathy 4, |
| | | | Cardiomyopathy |
| 397516138 | NM_000257.3(MYH7): | TATCAAYGAACTGTCCCTCAGGG, | Familial hypertrophic |
| | c.2206A > G (p.Ile736Val) | CTATCAAYGAACTGTCCCTCAGG | cardiomyopathy 1, |
| | | | Cardiomyopathy, not specified |
| 1154510 | NM_002150.2(HPD): | ATGACGYGGCCTGAATCACAGGG, | 4-Alpha- |
| | c.97G > A (p.Ala33Thr) | AATGACGYGGCCTGAATCACAGG | hydroxyphenylpyruvate |
| | | | hydroxylase deficiency |
| 397516330 | NM_000260.3(MYO7A): | ATATCCYGGGGGAGCAGAAAGGG, | Usher syndrome, type 1 |
| | c.6439 − 2A > G | GATATCCYGGGGGAGCAGAAAGG | |
| 72556271 | NM_000531.5(OTC): | CAGCCCAYTGATAATTGGGATGG | not provided |
| | c.482A > G (p.Asn161Ser) | | |
| 606231260 | NM_023073.3(C5orf42): | ATCYATCAAATACAAAAATTTGG | Orofaciodigital syndrome 6 |
| | c.3290 − 2A > G | | |
| 587777521 | NM_004817.3(TJP2): | CAGCTCYGAGAAGAAACCACGGG, | Progressive familial |
| | c.1992 − 2A > G | TCAGCTCYGAGAAGAAACCACGG | intrahepatic cholestasis 4 |
| 730880846 | NM_000257.3(MYH7): | CTTCYTGCTGCGGTCCCCAATGG | Cardiomyopathy |
| | c.617A > G (p.Lys206Arg) | | |
| 397517978 | NM_206933.2(USH2A): | TTCCCYGTAAGAAAATTAACAGG | Usher syndrome, type 2A, |
| | c.12067 − 2A > G | | Retinitis pigmentosa 39 |
| 606231409 | NM_000216.2(ANOS1): | GCACCAYGGCTGCGGGTCGAGGG, | Kallmann syndrome 1 |
| | c.1A > G (p.Met1Val) | GGCACCAYGGCTGCGGGTCGAGG | |
| 80356546 | NM_003334.3(UBA1): | TGGCYTGTCACCCGGATATGTGG | Arthrogryposis multiplex |
| | c.1639A > G (p.Ser547Gly) | | congenita, distal, X-linked |
| 80356584 | NM_194248.2(OTOF): | GACCYGCAGGCAGGAGAAGGGGG, | Deafness, autosomal recessive |
| | c.766 − 2A > G | TGACCYGCAGGCAGGAGAAGGGG, | 9 |
| | | CTGACCYGCAGGCAGGAGAAGGG, | |
| | | GCTGACCYGCAGGCAGGAGAAGG | |
| 730880930 | NM_000257.3(MYH7): | GGAACAYGCACTCCTCTTCCAGG | Cardiomyopathy |
| | c.1615A > G (p.Met539Val) | | |
| 118203947 | NM_013319.2(UBIAD1): | TCCYGTCATCACTCTTTTTGTGG | Schnyder crystalline corneal |
| | c.355A > G (p.Arg119Gly) | | dystrophy |
| 60171927 | NM_000526.4(KRT14): | GCGGTCAYTGAGGTTCTGCATGG | Epidermolysis bullosa |
| | c.368A > G (p.Asn123Ser) | | herpetiformis, Dowling-Meara |
| 199422248 | NM_001363.4(DKC1): | AATCYTGGCCCCATAGCAGATGG | Dyskeratosis congenita X- |
| | c.941A > G (p.Lys314Arg) | | linked |
| 72558467 | NM_000531.5(OTC): | TCCACTYCTTCTGGCTTTCTGGG, | not provided |
| | c.929A > G (p.Glu310Gly) | ATCCACTYCTTCTGGCTTTCTGG | |
| 72558478 | NM_000531.5(OTC): | ACTTTCYGTTTTCTGCCTCTGGG, | not provided |
| | c.988A > G (p.Arg330Gly) | CACTTTCYGTTTTCTGCCTCTGG | |
| 118204455 | NM_000505.3(F12): | GGTGGYACTGGAAGGGGAAGTGG | |
| | c.158A > G (p.Tyr53Cys) | | |
| 80357477 | NM_007294.3(BRCA1): | TTGYCCTCTGTCCAGGCATCTGG | Familial cancer of breast, |
| | c.5453A > G (p.Asp1818Gly) | | Breast-ovarian cancer, familial |
| | | | 1 |
| 121907908 | NM_024426.4(WT1): | CGCYCTCGTACCCTGTGCTGTGG | Mesothelioma |
| | c.1021A > G (p.Ser341Gly) | | |
| 121907926 | NM_000280.4(PAX6): | GTGGYGCCCGAGGTGCCCATTGG | Optic nerve aplasia, bilateral |
| | c.1171A > G (p.Thr391Ala) | | |
| 121908023 | NM_024740.2(ALG9): | TTAYACAAAACAATGTTGAGTGG | Congenital disorder of |
| | c.860A > G (p.Tyr287Cys) | | glycosylation type 1L |
| 121908148 | NM_001243133.1(NLRP3): | ACAATYCCAGCTGGCTGGGCTGG | Familial cold urticaria |
| | c.1880A > G (p.Glu627Gly) | | |
| 121908166 | NM_006492.2(ALX3): | CGGYTCTGGAACCAGACCTGGGG, | Frontonasal dysplasia 1 |
| | c.608A > G (p.Asn203Ser) | GCGGYTCTGGAACCAGACCTGGG, | |
| | | TGCGGYTCTGGAACCAGACCTGG | |
| 121908184 | NM_020451.2(SEPN1): | CCCAYGGCTGCGGCTGGCGGCGG, | Eichsfeld type congenital |
| | c.1A > G (p.Met1Val) | CGGCCCAYGGCTGCGGCTGGCGG | muscular dystrophy |
| 121908258 | NM_130468.3(CHST14): | AAGTCAYAGTGCACGGCACAAGG | Ehlers-Danlos syndrome, |
| | c.878A > G (p.Tyr293Cys) | | musculocontractural type |
| 121908383 | NM_001128425.1(MUTYH): | AAGCYGCTCTGAGGGCTCCCAGG | Neoplasm of stomach |
| | c.1241A > G (p.Gln414Arg) | | |
| 121908580 | NM_004328.4(BCS1L): | GTGYGATCATGTAATGGCGCCGG | Mitochondrial complex III |
| | c.148A > G (p.Thr50Ala) | | deficiency |
| 121908584 | NM_016417.2(GLRX5): | CCTGACCYTGTCGGAGCTCCGGG | Anemia, sideroblastic, |
| | c.294A > G (p.Gln98=) | | pyridoxine-refractory, |
| | | | autosomal recessive |
| 121908635 | NM_022817.2(PER2): | GCCACACYCTCTGCCTTGCCCGG | Advanced sleep phase |
| | c.1984A > G (p.Ser662Gly) | | syndrome, familial |
| 121908655 | NM_003839.3(TNFRSF11A): | GGGTCYGCATTTGTCCGTGGAGG | Osteopetrosis autosomal |
| | c.508A > G (p.Arg170Gly) | | recessive 7 |
| 29001653 | NM_000539.3(RHO): | CGCTCTYGGCAAAGAACGCTGGG, | Retinitis pigmentosa 4 |
| | c.886A > G (p.Lys296Glu) | GCGCTCTYGGCAAAGAACGCTGG | |
| 56307355 | NM_006502.2(POLH): | AGACTTTYCTGCTTAAAGAAGGG | Xeroderma pigmentosum, |
| | c.1603A > G (p.Lys535Glu) | | variant type |
| 121908919 | NM_002977.3(SCN9A): | CCTTTTCYTGTGTATTTGATTGG | Generalized epilepsy with |
| | c.1964A > G (p.Lys655Arg) | | febrile seizures plus, type 7, |
| | | | not specified |
| 121908939 | NM_006892.3(DNMT3B): | GACACGYCTGTGTAGTGCACAGG | Centromeric instability of |
| | c.2450A > G (p.Asp817Gly) | | chromosomes 1,9 and 16 and |
| | | | immunodeficiency |
| 121909088 | NM_001005360.2(DNM2): | ACTYCTTCTCTTTCTCCTGAGGG, | Charcot-Marie-Tooth disease, |
| | c.1684A > G (p.Lys562Glu) | TACTYCTTCTCTTTCTCCTGAGG | dominant intermediate b, with |
| | | | neutropenia |
| 120074112 | NM_000483.4(APOC2): | GCCCAYAGTGTCCAGAGACCTGG | Apolipoprotein C2 deficiency |
| | c.1A > G (p.Met1Val) | | |
| 121909239 | NM_000314.6(PTEN): | ATAYCACCACACACAGGTAACGG | Macrocephaly/autism |
| | c.755A > G (p.Asp252Gly) | | syndrome |
| 121909251 | NM_198217.2(ING1): | TGGYTGCACAGACAGTACGTGGG, | Squamous cell carcinoma of |
| | c.515A > G (p.Asn172Ser) | CTGGYTGCACAGACAGTACGTGG | the head and neck |
| 121909396 | NM_001174089.1(SLC4A11): | GATCAYCTTCATGTAGGGCAGGG, | Corneal dystrophy and |
| | c.2518A > G (p.Met840Val) | AGATCAYCTTCATGTAGGGCAGG | perceptive deafness |
| 121909533 | NM_000034.3(ALDOA): | CCAYCCAACCCTAAGAGAAGAGG | HNSHA due to aldolase A |
| | c.386A > G (p.Asp129Gly) | | deficiency |
| 128627255 | NM_004006.2(DMD): | TGACCGYGATCTGCAGAGAAGGG, | Dilated cardiomyopathy 3B |
| | c.835A > G (p.Thr279Ala) | CTGACCGYGATCTGCAGAGAAGG | |
| 116929575 | NM_001085.4(SERPINA3): | GCTCAYGAAGAAGATGTTCTGGG, | |
| | c.1240A > G (p.Met414Val) | TGCTCAYGAAGAAGATGTTCTGG | |
| 61748392 | NM_004992.3(MECP2): | CAACYCCACTTTAGAGCGAAAGG | Mental retardation, X-linked, |
| | c.410A > G (p.Glu137Gly) | | syndromic 13 |
| 61748906 | NM_001005741.2(GBA): | CCCACTYGGCTCAAGACCAATGG | Gaucher disease, type 1 |
| | c.667T > C (p.Trp223Arg) | | |
| 199473024 | NM_000238.3(KCNH2): | CTGCYCTCCACGTCGCCCCGGGG, | Sudden infant death syndrome |
| | c.3118A > G (p.Ser1040Gly) | CCTGCYCTCCACGTCGCCCCGGG, | |
| | | GCCTGCYCTCCACGTCGCCCCGG | |
| 794728365 | NM_000238.3(KCNH2): | GGACCYGCACCCGGGGAAGGCGG | Cardiac arrhythmia |
| | c.1129 − 2A > G | | |
| 72556293 | NM_000531.5(OTC): | AGAGCTAYAGTGTTCCTAAAAGG | not provided |
| | c.548A > G (p.Tyr183Cys) | | |
| 111033244 | NM_000441.1(SLC26A4): | TGAATYCCTAAGGAAGAGACTGG | Pendred syndrome, Enlarged |
| | c.1151A > G (p.Glu384Gly) | | vestibular aqueduct syndrome |
| 111033415 | NM_000260.3(MYO7A): | AGCYGCAGGGGCACAGGGATGGG, | Usher syndrome, type 1 |
| | c.1344 − 2A > G | AAGCYGCAGGGGCACAGGGATGG | |
| 121912439 | NM_000454.4(SOD1): | AGAATCTYCAATAGACACATCGG | Amyotrophic lateral sclerosis |
| | c.302A > G (p.Glu101Gly) | | type 1 |
| 111033567 | NM_002769.4(PRSS1): | ATCYTGTCATCATCATCAAAGGG, | Hereditary pancreatitis |
| | c.68A > G (p.Lys23Arg) | GATCYTGTCATCATCATCAAAGG | |
| 121912565 | NM_000901.4(NR3C2): | TCATCYGTTTGCCTGCTAAGCGG | Pseudohypoaldosteronism type |
| | c.2327A > G (p.Gln776Arg) | | 1 autosomal dominant |
| 121912574 | NM_000901.4(NR3C2): | CCGACYCCACCTTGGGCAGCTGG | Pseudohypoaldosteronism type |
| | c.2915A > G (p.Glu972Gly) | | 1 autosomal dominant |
| 121912589 | NM_001173464.1(KIF21A): | ATTCAYATCTGCCTCCATGTTGG | Fibrosis of extraocular |
| | c.2839A > G (p.Met947Val) | | muscles, congenital, 1 |
| 111033661 | NM_000155.3(GALT): | ATTCACCYACCGACAAGGATAGG | Deficiency of UDPglucose- |
| | c.253 − 2A > G | | hexose-1-phosphate |
| | | | uridylyltransferase |
| 111033669 | NM_000155.3(GALT): | GAAGTCGYTGTCAAACAGGAAGG | Deficiency of UDPglucose- |
| | c.290A > G (p.Asn97Ser) | | hexose-1-phosphate |
| | | | uridylyltransferase |
| 111033682 | NM_000155.3(GALT): | TGACCTYACTGGGTGGTGACGGG, | Deficiency of UDPglucose- |
| | c.379A > G (p.Lys127Glu) | ATGACCTYACTGGGTGGTGACGG | hexose-1-phosphate |
| | | | uridylyltransferase |
| 111033786 | NM_000155.3(GALT): | CAGCYGCCAATGGTTCCAGTTGG | Deficiency of UDPglucose- |
| | c.950A > G (p.Gln317Arg) | | hexose-1-phosphate |
| | | | uridylyltransferase |
| 121912765 | NM_001202.3(BMP4): | CCTCCYCCCCAGACTGAAGCCGG | Microphthalmia syndromic 6 |
| | c.278A > G (p.Glu93Gly) | | |
| 121912856 | NM_000094.3(COL7A1): | CACCYTGGGGACACCAGGTCGGG, | Epidermolysis bullosa |
| | c.425A > G (p.Lys142Arg) | TCACCYTGGGGACACCAGGTCGG | dystrophica inversa, autosomal |
| | | | recessive |
| 199474715 | NM_152263.3(TPM3): | CCAACTYACGAGCCACCTACAGG | Congenital myopathy with |
| | c.505A > G (p.Lys169Glu) | | fiber type disproportion |
| 199474718 | NM_152263.3(TPM3): | ATCYCTCAGCAAACTCAGCACGG | Congenital myopathy with |
| | c.733A > G (p.Arg245Gly) | | fiber type disproportion |
| 121912895 | NM_001844.4(COL2A1): | CCTCYCTCACCACGTTGCCCAGG | Spondyloepimetaphyseal |
| | c.2974A > G (p.Arg992Gly) | | dysplasia Strudwick type |
| 121913074 | NM_000129.3(F13A1): | ATAGGCAYAGATATTGTCCCAGG | Factor xiii, a subunit, |
| | c.851A > G (p.Tyr284Cys) | | deficiency of |
| 121913145 | NM_000208.2(INSR): | GCTGYGGCAACAGAGGCCTTCGG | Leprechaunism syndrome |
| | c.707A > G (p.His236Arg) | | |
| 312262745 | NM_025137.3(SPG11): | ACTTAYCCTGGGGAGAAGGATGG | Spastic paraplegia 11, |
| | c.2608A > G (p.Ile870Val) | | autosomal recessive |
| 121913682 | NM_000222.2(KIT): | AGAAYCATTCTTGATGTCTCTGG | Mast cell disease, systemic |
| | c.2459A > G (p.Asp820Gly) | | |
| 587776757 | NM_000151.3(G6PC): | GTTCYTACCACTTAAAGACGAGG | Glycogen storage disease type |
| | c.230 + 4A > G | | 1A |
| 61752063 | NM_000330.3(RS1): | TTCTTCGYGGACTGCAAACAAGG | Juvenile retinoschisis |
| | c.286T > C (p.Trp96Arg) | | |
| 367543065 | NM_024549.5(TCTN1): | AGCAACYGCAGAAAAAAGAGGGG, | Joubert syndrome 13 |
| | c.221 − 2A > G | CAGCAACYGCAGAAAAAAGAGGG | |
| 5030773 | NM_000894.2(LHB): | CCACCYGAGGCAGGGGCGGCAGG | Isolated lutropin deficiency |
| | c.221A > G (p.Gln74Arg) | | |
| 199476092 | NM_000264.3(PTCH1): | CGTTACYGAAACTCCTGTGTAGG | Gorlin syndrome, |
| | c.2479A > G (p.Ser827Gly) | | Holoprosencephaly 7, not |
| | | | specified |
| 398123158 | NM_000117.2(EMD): | CGTTCCCYGAGGCAAAAGAGGGG | not provided |
| | c.450 − 2A > G | | |
| 199476103 | RMRP:n.71A > G | ACTTYCCCCTAGGCGGAAAGGGG, | Metaphyseal |
| | | GACTTYCCCCTAGGCGGAAAGGG, | chondrodysplasia, McKusick |
| | | GGACTTYCCCCTAGGCGGAAAGG | type, Metaphyseal dysplasia |
| | | | without hypotrichosis |
| 5030856 | NM_000277.1(PAH): | CTCYCTGCCACGTAATACAGGGG, | Phenylketonuria, |
| | c.1169A > G (p.Glu390Gly) | ACTCYCTGCCACGTAATACAGGG, | Hyperphenylalaninemia, non- |
| | | AACTCYCTGCCACGTAATACAGG | pku |
| 5030860 | NM_000277.1(PAH): | GGGTCGYAGCGAACTGAGAAGGG, | Phenylketonuria, |
| | c.1241A > G (p.Tyr414Cys) | TGGGTCGYAGCGAACTGAGAAGG | Hyperphenylalaninemia, non- |
| | | | pku |
| 587777055 | NM_020988.2(GNAO1): | GGATGYCCTGCTCGGTGGGCTGG | Early infantile epileptic |
| | c.521A > G (p.Asp174Gly) | | encephalopathy 17 |
| 587777223 | NM_024301.4(FKRP): | CCGCAYGGGGCCGAAGTCTGGGG, | Congenital muscular |
| | c.1A > G (p.Met1Val) | GCCGCAYGGGGCCGAAGTCTGGG, | dystrophy-dystroglycanopathy |
| | | AGCCGCAYGGGGCCGAAGTCTGG | with brain and eye anomalies |
| | | | type A5 |
| 587777479 | NM_003108.3(SOX11): | GTACTTGYAGTCGGGGTAGTCGG | Mental retardation, autosomal |
| | c.347A > G (p.Tyr116Cys) | | dominant 27 |
| 587777496 | NM_020435.3(GJC2): | TTGYTCCCCCCTCGGCCTCAGGG, | Leukodystrophy, |
| | c.−170A > G | ATTGYTCCCCCCTCGGCCTCAGG | hypomyelinating, 2 |
| 587777507 | NM_022552.4(DNMT3A): | CTCCYGGTGCTGAAGGACTTGGG, | Tatton-Brown-rahman |
| | c.1943T > C (p.Leu648Pro) | GCTCCYGGTGCTGAAGGACTTGG | syndrome |
| 587777557 | NM_018400.3(SCN3B): | AATCAYGATGTACATCCTTCTGG | Atrial fibrillation, familial, |
| | c.482T > C (p.Met161Thr) | | 16 |
| 587777569 | NM_001030001.2(RPS29): | GATAYCGGTTTCATTAAGGTAGG | Diamond-Blackfan anemia 13 |
| | c.149T > C (p.Ile50Thr) | | |
| 587777657 | NM_153334.6(SCARF2): | CCACGYGCTGCGCTGGCTGGAGG | Marden Walker like syndrome |
| | c.190T > C (p.Cys64Arg) | | |
| 587777689 | NM_005726.5(TSFM): | ACTTCYCACCGGGTAGCTCCCGG | Combined oxidative |
| | c.57 + 4A > G | | phosphorylation deficiency 3 |
| 796052005 | NM_000255.3(MUT): | GCAYACTGGCGGATGGTCCAGGG, | not provided |
| | c.329A > G (p.Tyr110Cys) | AGCAYACTGGCGGATGGTCCAGG | |
| 587777809 | NM_144596.3(TTC8): | GTTCCYGGAAAGCATTAAGAAGG | Retinitis pigmentosa 51 |
| | c.115 − 2A > G | | |
| 587777878 | NM_000166.5(GJB1): | TAGCAYGAAGACGGTGAAGACGG | X-linked hereditary motor and |
| | c.580A > G (p.Met194Val) | | sensory neuropathy |
| 74315420 | NM_001029871.3(RSPO4): | CGTACYGGCGGATGCCTTCCCGG | Anonychia |
| | c.194A > G (p.Gln65Arg) | | |
| 180177219 | NM_000030.2(AGXT): | AGGCCCYGAGGAAGCAGGGACGG | Primary hyperoxaluria, type I |
| | c.424 − 2A > G | | |
| | (p.Gly_142Gln145del) | | |
| 367610201 | NM_002693.2(POLG): | CTCAYGGCACTTACCTGGGATGG | not provided |
| | c.1808T > C (p.Met603Thr) | | |
| 180177319 | NM_012203.1(GRHPR): | TCACAGCYGCGGGGAAAGGGAGG | Primary hyperoxaluria, type II |
| | c.84 − 2A > G | | |
| 796052068 | NM_000030.2(AGXT): | GGTACCYGGAAGACACGAGGGGG, | Primary hyperoxaluria, type I |
| | c.777 − 2A > G | TGGTACCYGGAAGACACGAGGGG | |
| 61754010 | NM_000552.3(VWF): | TGCCAYTGTAATTCCCACACAGG | von Willebrand disease, type |
| | c.1583A > G (p.Asn528Ser) | | 2a |
| 587778866 | NM_000321.2(RB1): | ATTYCAATGGCTTCTGGGTCTGG | Retinoblastoma |
| | c.1927A > G (p.Lys643Glu) | | |
| 74435397 | NM_006331.7(EMG1): | ATAYCTGGCCGCGCTTCCCCAGG | Bowen-Conradi syndrome |
| | c.257A > G (p.Asp86Gly) | | |
| 796052527 | NM_000156.5(GAMT): | CGCTCAYGCTGCAGGCTGGACGG | not provided |
| | c.1A > G (p.Met1Val) | | |
| 796052637 | NM_172107.2(KCNQ2): | GTACYTGTCCCCGTAGCCAATGG | not provided |
| | c.848A > G (p.Lys283Arg) | | |
| 724159963 | NM_032228.5(FAR1): | GATAYCATACAGGAATGCTGGGG, | Peroxisomal fatty acyl-coa |
| | c.1094A > G (p.Asp365Gly) | AGATAYCATACAGGAATGCTGGG, | reductase 1 disorder |
| | | TAGATAYCATACAGGAATGCTGG | |
| 587779722 | NM_000090.3(COL3A1): | CACCCYAAAGAAGAAGTGGTCGG | Ehlers-Danlos syndrome, type |
| | c.1762 − 2A > G | | 4 |
| | (p.Gly588_Gln605del) | | |
| 118192102 | m.8296A > G | TTTACAGYGGGCTCTAGAGGGGG | Diabetes-deafness syndrome |
| | | | maternally transmitted |
| 727502787 | NM_001077494.3(NFKB2): | CTGYCTTCCTTCACCTCTGCTGG | Common variable |
| | c.2594A > G (p.Asp865Gly) | | immunodeficiency 10 |
| 727503036 | NM_000117.2(EMD): | AGCCYTGGGAAGGGGGGCAGCGG | Emery-Dreifuss muscular |
| | c.266 − 2A > G | | dystrophy 1, X-linked |
| 690016544 | NM_005861.3(STUB1): | GGCCCGGYTGGTGTAATACACGG | Spinocerebellar ataxia, |
| | c.194A > G (p.Asn65Ser) | | autosomal recessive 16 |
| 690016554 | NM_005211.3(CSF1R): | GTATCYGGGAGATAGGACAGAGG | Hereditary diffuse |
| | c.2655 − 2A > G | | leukoencephalopathy with |
| | | | spheroids |
| 118192185 | NM_172107.2(KCNQ2): | GCACCAYGGTGCCTGGCGGGAGG | Benign familial neonatal |
| | c.1A > G (p.Met1Val) | | seizures 1 |
| 121917869 | NM_012064.3(MIP): | AGATCYCCACTGTGGTTGCCTGG | Cataract 15, multiple types |
| | c.401A > G (p.Glu134Gly) | | |
| 121918014 | NM_000478.4(ALPL): | AGGCCCAYTGCCATACAGGATGG | Infantile hypophosphatasia |
| | c.1250A > G (p.Asn417Ser) | | |
| 121918036 | NM_000174.4(GP9): | GCAGYCCACCCACAGCCCCATGG | Bernard-Soulier syndrome |
| | c.110A > G (p.Asp37Gly) | | type C |
| 121918089 | NM_000371.3(TTR): | CGGCAAYGGTGTAGCGGCGGGGG, | Amyloidogenic transthyretin |
| | c.379A > G (p.Ile127Val) | GCGGCAAYGGTGTAGCGGCGGGG | amyloidosis |
| 121918121 | NM_000823.3(GHRHR): | CGACTYGGAGAGACGCCTGCAGG | Isolated growth hormone |
| | c.985A > G (p.Lys329Glu) | | deficiency type 1B |
| 121918333 | NM_015335.4(MED13L): | ATATCAYCTAGAGGGAAGGGGGG, | Transposition of great arteries |
| | c.6068A > G (p.Asp2023Gly) | CATATCAYCTAGAGGGAAGGGGG | |
| 121918605 | NM_001035.2(RYR2): | CGCCAGCYGCATTTCAAAGATGG | Catecholaminergic |
| | c.12602A > G | | polymorphic ventricular |
| | (p.Gln4201Arg) | | tachycardia |
| 587781262 | NM_002764.3(PRPS1): | TAGCAYATTTGCAACAAGCTTGG | Charcot-Marie-Tooth disease, |
| | c.343A > G (p.Met115Val) | | X-linked recessive, type 5, |
| | | | Deafness, high-frequency |
| | | | sensorineural, X-linked |
| 121918608 | NM_001161766.1(AHCY): | GCGGGYACTTGGTGTGGATGAGG | Hypermethioninemia with s- |
| | c.344A > G (p.Tyr115Cys) | | adenosylhomocysteine |
| | | | hydrolase deficiency |
| 121918613 | NM_000702.3(ATP1A2): | CTGYCAGGGTCAGGCACACCTGG | Familial hemiplegic migraine |
| | c.1033A > G (p.Thr345Ala) | | type 2 |
| 587781339 | NM_000535.5(PMS2): | GCAGACCYGCACAAAATACAAGG | Hereditary cancer-predisposing |
| | c.904 − 2A > G | | syndrome |
| 121918691 | NM_001128177.1(THRB): | CTTCAYGTGCAGGAAGCGGCTGG | Thyroid hormone resistance, |
| | c.1324A > G (p.Met442Val) | | generalized, autosomal |
| | | | dominant |
| 121918692 | NM_001128177.1(THRB): | CCACCTYCATGTGCAGGAAGCGG | Thyroid hormone resistance, |
| | c.1327A > G (p.Lys443Glu) | | generalized, autosomal |
| | | | dominant |
| 727504333 | NM_000256.3(MYBPC3): | CCGTTCYGTGGGTATAGAGTGGG, | Familial hypertrophic |
| | c.2906 − 2A > G | GCCGTTCYGTGGGTATAGAGTGG | cardiomyopathy 4 |
| 730880805 | NM_000257.3(MYH7): | GCCCYCCTCGTGCTCCAGGGAGG, | Cardiomyopathy |
| | c.4664A > G (p.Glu1555Gly) | CTTGCCCYCCTCGTGCTCCAGGG | |
| 267606834 | NM_138387.3(G6PC3): | TGATCAYGCAGTGTCCAGAAGGG, | Dursun syndrome |
| | c.346A > G (p.Met116Val) | GTGATCAYGCAGTGTCCAGAAGG | |
| 267606851 | NM_000175.3(GPI): | GTACYGGTCATAGGGCAGCATGG | Hemolytic anemia, |
| | c.1028A > G (p.Gln343Arg) | | nonspherocytic, due to glucose |
| | | | phosphate isomerase |
| | | | deficiency |
| 515726182 | NM_015713.4(RRM2B): | TTCCTTCYGGACAGCAGAAGAGG | RRM2B-related mitochondrial |
| | c.190T > C (p.Trp64Arg) | | disease |
| 730881002 | NM_002880.3(RAF1): | GCTGCYGCCCTCGCACCACTGGG, | Rasopathy |
| | c.1279A > G (p.Ser427Gly) | GGCTGCYGCCCTCGCACCACTGG | |
| 267607030 | NM_002977.3(SCN9A): | AAGCTCYGAGGTCCTGGGGGAGG | Primary erythromelalgia |
| | c.29A > G (p.Gln10Arg) | | |
| 267607048 | NM_007373.3(SHOC2): | TACYCATGGTGACTCAAGCCTGG | Noonan-like syndrome with |
| | c.4A > G (p.Ser2Gly) | | loose anagen hair, Rasopathy |
| 587783486 | NM_004380.2(CREBBP): | GCAGCCCYAGGAAGTCCAGAAGG | Rubinstein-Taybi syndrome |
| | c.3983 − 2A > G | | |
| 730881357 | NM_000051.3(ATM): | AGCCYACGGGAAAAGAACTGTGG | Hereditary cancer-predisposing |
| | c.3154 − 2A > G | | syndrome |
| 398122404 | NM_001256864.1(DNAJC6): | AGGTATCYGAAACAGAAGGTTGG | Parkinson disease 19, juvenile- |
| | c.801 − 2A > G | | onset |
| 267607482 | NM_001927.3(DES): | GAATCGTYCTGCAGGAGAGGGGG | Myofibrillar myopathy 1 |
| | c.1024A > G (p.Asn342Asp) | | |
| 796053439 | NM_000391.3(TPP1): | CAGGTACYGCACATCTAGACTGG | not provided |
| | c.833A > G (p.Gln278Arg) | | |
| 587783835 | NM_000252.2(MTM1): | GTTATTCYCCAATGGTGATTGGG | Severe X-linked myotubular |
| | c.550A > G (p.Arg184Gly) | | myopathy |
| 587783842 | NM_000252.2(MTM1): | TCATCAYCTGAGGCACGATACGG | Severe X-linked myotubular |
| | c.629A > G (p.Asp210Gly) | | myopathy |
| 267607777 | NM_000249.3(MLH1): | TGCTACAYTACCTGAGGTACAGG | Hereditary Nonpolyposis |
| | c.884 + 4A > G | | Colorectal Neoplasms |
| 33972047 | NM_000518.4(HBB): | CACGYTCACCTTGCCCCACAGGG, | alpha Thalassemia |
| | c.59A > G (p.Asn20Ser) | CCACGYTCACCTTGCCCCACAGG | |
| 730882004 | NM_000546.5(TP53): | ACACAYGTAGTTGTAGTGGATGG | Li-Fraumeni syndrome, |
| | c.709A > G (p.Met237Val) | | Hereditary cancer- |
| | | | predisposing syndrome |
| 730882052 | NM_001231.4(CASQ1): | GGCTTGYCTGGGATGGTCACAGG | Myopathy, vacuolar, with |
| | c.731A > G (p.Asp244Gly) | | casq1 aggregates |
| 80338959 | NM_000334.4(SCN4A): | GATCAYGATGGTGATGTCGAAGG | Hyperkalemic Periodic |
| | c.4078A > G (p.Met1360Val) | | Paralysis Type 1 |
| 80338960 | NM_000334.4(SCN4A): | CCATCAYGGTGACCATGTTGAGG | Hyperkalemic Periodic |
| | c.4108A > G (p.Met1370Val) | | Paralysis Type 1 |
| 80338962 | NM_000334.4(SCN4A): | TGTACAYGTTGACCACGATGAGG | Hyperkalemic Periodic |
| | c.4774A > G (p.Met1592Val) | | Paralysis Type 1, Familial |
| | | | hyperkalemic periodic |
| | | | paralysis |
| 398123062 | NM_012160.4(FBXL4): | TATGYCCAGCTGCTGTAACCTGG | Mitochondrial DNA depletion |
| | c.1694A > G (p.Asp565Gly) | | syndrome 13 |
| | | | (encephalomyopathic type) |
| 730882140 | NM_001039550.1(DNAJB2): | GATCTCGYAGTAGGATGCCATGG | Charcot-Marie-Tooth disease, |
| | c.14A > G (p.Tyr5Cys) | | Charcot-Marie-Tooth disease, |
| | | | axonal, type 2T |
| 796053522 | NM_052859.3(RFT1): | GCAYCACAAAATTGTACCTGGGG, | Congenital disorder of |
| | c.1222A > G (p.Met408Val) | AGCAYCACAAAATTGTACCTGGG, | glycosylation type 1N |
| | | CAGCAYCACAAAATTGTACCTGG | |
| 398123211 | NM_000169.2(GLA): | AACCYGTATGAGAAAACAATGGG, | Fabry disease |
| | c.548 − 2A > G | TAACCYGTATGAGAAAACAATGG | |
| 587784423 | NM_006306.3(SMC1A): | AGCCYGTGCAAACAGGGGAATGG | Congenital muscular |
| | c.616 − 2A > G | | hypertrophy-cerebral |
| | | | syndrome |
| 398123411 | NM_000487.5(ARSA): | GGCTCYGGGGGCAGAGTCAGGGG, | Metachromatic |
| | c.1108 − 2A > G | GGGCTCYGGGGGCAGAGTCAGGG, | leukodystrophy |
| | | AGGGCTCYGGGGGCAGAGTCAGG | |
| 398123429 | NM_000512.4(GALNS): | CCGCCAYCAGCGTGTCGCCACGG | Mucopolysaccharidosis, MPS- |
| | c.1171A > G (p.Met391 Val) | | IV-A |
| 267608500 | NM_003159.2(CDKL5): | ATGYCCACGGACTTTCCATAGGG, | Early infantile epileptic |
| | c.578A > G (p.Asp193Gly) | CATGYCCACGGACTTTCCATAGG | encephalopathy 2 |
| 398123552 | NM_000402.4(G6PD): | ACACACAYATTCATCATCATGGG | Anemia, nonspherocytic |
| | c.188T > C (p.Ile63Thr) | | hemolytic, due to G6PD |
| | | | deficiency |
| 75391579 | NM_000155.3(GALT): | TTACCYGGCAGTGGGGGTGGGGG, | Deficiency of UDPglucose- |
| | c.563A > G (p.Gln188Arg) | CTTACCYGGCAGTGGGGGTGGGG, | hexose-1-phosphate |
| | | CCTTACCYGGCAGTGGGGGTGGG | uridylyltransferase |
| 398123639 | NM_001848.2(COL6A1): | TTCTCCCYGGAACACAAAACAGG | Ullrich congenital muscular |
| | c.805 − 2A > G | | dystrophy, Bethlem myopathy |
| 398123750 | NM_003482.3(KMT2D): | GCAGTTCYGTGGGGGAATGAAGG | Kabuki make-up syndrome |
| | c.5645 − 2A > G | | |
| 398124528 | NM_144997.5(FLCN): | CCCACYGGGGAGAAGGGCAGGGG, | Hereditary cancer- |
| | c.1433 − 2A > G | GCCCACYGGGGAGAAGGGCAGGG, | predisposing syndrome |
| | | GGCCCACYGGGGAGAAGGGCAGG | |
| 113994149 | NM_025265.3(TSEN2): | CAGAGCAYAGACCAAGAAAAAGG | Pontocerebellar hypoplasia |
| | c.926A > G (p.Tyr309Cys) | | type 2B |
| 281865052 | NM_198578.3(LRRK2): | TCAACAYAATATTTCTAGGCAGG | Parkinson disease 8, autosomal |
| | c.5605A > G (p.Met1869Val) | | dominant |
| 281865495 | NM_004614.4(TK2): | AAGYCTCAGGATTGGTCCGAAGG | Mitochondrial DNA depletion |
| | c.562A > G (p.Thr188Ala) | | syndrome 2 |
| 786200910 | NM_006204.3(PDE6C): | CTTTCYGTTGAAATAAGGATGGG, | Achromatopsia 5 |
| | c.1483 − 2A > G | TCTTTCYGTTGAAATAAGGATGG | |
| 281860296 | NM_000551.3(VHL): | GGTCTTYCTGCACATTTGGGTGG | Von Hippel-Lindau syndrome |
| | c.586A > T (p.Lys196Ter) | | |
| 730880444 | NM_000169.2(GLA): | GTGAACCYGAAATGAGAGGGAGG | not provided |
| | c.370 − 2A > G | | |
| 756328339 | NM_003494.3(DYSF): | CTAYACTCCCAGCCTGGGGGAGG, | Limb-girdle muscular |
| | c.3041A > G (p.Tyr1014Cys) | ATGCTAYACTCCCAGCCTGGGGG, | dystrophy, type 2B |
| | | GATGCTAYACTCCCAGCCTGGGG | |
| 387906810 | NM_153427.2(PITX2): | TCTYGAACCAAACCTGGGGGCGG, | Axenfeld-Rieger syndrome |
| | c.262A > G (p.Lys88Glu) | GATTCTYGAACCAAACCTGGGGG, | type 1 |
| | | CGATTCTYGAACCAAACCTGGGG | |
| 78310959 | NM_030964.3(SPRY4): | AGTGCYTGTCCAGCTCGGGTGGG, | Hypogonadotropic |
| | c.530A > G (p.Lys177Arg) | AAGTGCYTGTCCAGCTCGGGTGG | hypogonadism 17 with or |
| | | | without anosmia |
| 144109267 | NM_207352.3(CYP4V2): | TTCCYGGGGCCAGCAGAGAAGGG, | Bietti crystalline corneoretinal |
| | c.1393A > G (p.Arg465Gly) | GTTCCYGGGGCCAGCAGAGAAGG | dystrophy |
| 104886319 | NM_000495.4(COL4A5): | CACCYGAGTAAGATAAAGAAAGG | Alport syndrome, X-linked |
| | c.1340 − 2A > G | | recessive |
| 104886416 | NM_000495.4(COL4A5): | ACCCYAAAAGAAGCCATCAATGG | Alport syndrome, X-linked |
| | c.466 − 2A > G | | recessive |
| 121434443 | NM_004984.2(KIF5A): | GAACAYAGCTTTTCTGGGGGAGG | Spastic paraplegia 10 |
| | c.827A > G (p.Tyr276Cys) | | |
| 199422314 | NM_001099274.1(TINF2): | TGACTGYGGGGCGCTCCTTATGG | Dyskeratosis congenita |
| | c.850A > G (p.Thr284Ala) | | autosomal dominant |
| 121434478 | NM_004044.6(ATIC): | AGTGTACYTGACAGCAATGGTGG | AICAR transformylase/IMP |
| | c.1277A > G (p.Lys426Arg) | | cyclohydrolase deficiency |
| 111033765 | NM_000155.3(GALT): | CGCYCAGCAGGGGTCAGCTCAGG | Deficiency of UDPglucose- |
| | c.812A > G (p.Glu271Gly) | | hexose-1-phosphate |
| | | | uridylyltransferase |
| 121434606 | NM_006006.4(ZBTB16): | GATCAYGGCCGAGTAGTCCCGGG, | Skeletal defects, genital |
| | c.1849A > G (p.Met617Val) | TGATCAYGGCCGAGTAGTCCCGG | hypoplasia, and mental |
| | | | retardation |
| 566325901 | NM_000017.3(ACADS): | AGCCCAYGCCGCCCAGGATCTGG | not provided |
| | c.1108A > G (p.Met370Val) | | |
| 148665132 | NM_012079.5(DGAT1): | ACCGCGGYGAGGACCTCTGTGGG | Diarrhea 7 |
| | c.751 + 2T > C | | |
| 111033830 | NM_000155.3(GALT): | TGCYGGCCCATACCTGTCAAGGG, | Deficiency of UDPglucose- |
| | c.574A > G (p.Ser192Gly) | CTGCYGGCCCATACCTGTCAAGG | hexose-1-phosphate |
| | | | uridylyltransferase |
| 28933679 | NM_000132.3(F8): | GAGYGCACATCTTTTTCCTAGGG, | Hereditary factor VIII |
| | c.5600A > G (p.His1867Arg) | TGAGYGCACATCTTTTTCCTAGG | deficiency disease |
| 137852251 | NM_000133.3(F9): | GCTGCAYTGTAGTTGTGGTGAGG | Hereditary factor IX deficiency |
| | c.917A > G (p.Asn306Ser) | | disease |
| 141686175 | NM_001287223.1(SCN11A): | CGTGCGCYGTCCCAGTTTGAAGG | Episodic pain syndrome, |
| | c.3473T > C (p.Leu1158Pro) | | familial, 3 |
| 137852331 | NM_000402.4(G6PD): | ATGCGGTYCCAGCCTCTGCTGGG | Favism, susceptibility to, |
| | c.583A > G (p.Asn195Asp) | | Anemia, nonspherocytic |
| | | | hemolytic, due to G6PD |
| | | | deficiency |
| 137852369 | NM_000132.3(F8): | TAGCCATYGATTGCTGGAGAAGG | Hereditary factor VIII |
| | c.5821A > G (p.Asn1941Asp) | | deficiency disease |
| 137852389 | NM_000132.3(F8): | TCAYATTCAGCTCCTATAGCAGG | Hereditary factor VIII |
| | c.398A > G (p.Tyr133Cys) | | deficiency disease |
| 137852406 | NM_000132.3(F8): | TGAGCAGYAAGGAAAGTTATTGG | Hereditary factor VIII |
| | c.940A > G (p.Thr314Ala) | | deficiency disease |
| 28931576 | NM_000041.3(APOE): | ACAGTGYCTGCACCCAGCGCAGG | |
| | c.178A > G (p.Thr60Ala) | | |
| 74315301 | NM_000396.3(CTSK): | GAGYCACATCTTGGGGAAGCTGG | Pyknodysostosis |
| | c.990A > G (p.Ter330Trp) | | |
| 137852540 | NM_002764.3(PRPS1): | TAGCATAYTTGCAACAAGCTTGG | Phosphoribosylpyrophosphate |
| | c.341A > G (p.Asn114Ser) | | synthetase superactivity |
| 137852624 | NM_000215.3(JAK3): | AATCCTGYACAGCAGGACTTGGG | Severe combined |
| | c.299A > G (p.Tyr100Cys) | | immunodeficiency, autosomal |
| | | | recessive, T cell-negative, B |
| | | | cell-positive, NK cell-negative |
| 137852640 | NM_001166107.1(HMGCS2): | ACCACCGYAGCAGGCATTGGTGG | mitochondrial 3-hydroxy-3- |
| | c.500A > G (p.Tyr167Cys) | | methylglutaryl-CoA synthase |
| | | | deficiency |
| 137852814 | NM_005633.3(SOS1): | GCATCCYTTCCAGTGTACTCCGG | Noonan syndrome, Noonan |
| | c.1654A > G (p.Arg552Gly) | | syndrome 4, Rasopathy |
| 137852865 | NM_001171993.1(HPD): | CCTCAYATCCAGGCAAGAATTGG | 4-Hydroxyphenylpyruvate |
| | c.362A > G (p.Tyr121Cys) | | dioxygenase deficiency |
| 370898981 | NM_138691.2(TMC1): | TGGCCYACCAGATCATGCCTTGG | Deafness, autosomal recessive |
| | c.1763 + 3A > G | | 7 |
| 118192167 | NM_000540.2(RYR1): | CCATAYACCAGCCCAGGTACAGG | Malignant hyperthermia |
| | c.14387A > G | | susceptibility type 1, Central |
| | (p.Tyr4796Cys) | | core disease |
| 137852972 | NM_032667.6(BSCL2): | CGAGACAYTGGCAACAGGGAAGG | Distal hereditary motor |
| | c.263A > G (p.Asn88Ser) | | neuronopathy type 5, Silver |
| | | | spastic paraplegia syndrome, |
| | | | Charcot-Marie-Tooth disease, |
| | | | type 2 |
| 118192193 | NM_172107.2(KCNQ2): | CTTCYCATACTCCTTGATGGTGG, | Benign familial neonatal |
| | c.356A > G (p.Glu119Gly) | GCTCTTCYCATACTCCTTGATGG | seizures 1 |
| 118192201 | NM_172107.2(KCNQ2): | GGATCAYCCGCAGAATCTGCAGG | Benign familial neonatal |
| | c.622A > G (p.Met208Val) | | seizures 1 |
| 137853027 | NM_001080463.1(DYNC2H1): | ATAYCTCTAATTACATCAGGTGG, | Short-rib thoracic dysplasia 3 |
| | c.9044A > G | AGAATAYCTCTAATTACATCAGG | with or without polydactyly |
| | (p.Asp3015Gly) | | |
| 137853197 | NM_144573.3(NEXN): | ATAYACTCTCCTCCATCTTCTGG | Dilated cardiomyopathy 1CC, |
| | c.1955A > G (p.Tyr652Cys) | | Cardiomyopathy, not specified |
| 137853203 | NM_000476.2(AK1): | TTCTCAYAGAAGGCGATGACGGG, | Adenylate kinase deficiency, |
| | c.491A > G (p.Tyr164Cys) | TTTCTCAYAGAAGGCGATGACGG | hemolytic anemia due to |
| 786200859 | NM_000308.2(CTSA): | TCCCAYACCTGTTCCCCAGAAGG | Galactosialidosis, adult |
| | c.746 + 3A > G | | |
| 786200897 | NM_003494.3(DYSF): | CAGCYAGAAGACACAGGGAGGGG, | Limb-girdle muscular |
| | c.1285 − 2A > G | ACAGCYAGAAGACACAGGGAGGG, | dystrophy, type 2B |
| | | CACAGCYAGAAGACACAGGGAGG | |
| 786200928 | NM_206933.2(USH2A): | CTCTTAYCTTGGGAAAGGAGAGG | Usher syndrome, type 2A |
| | c.7595 − 2144A > G | | |
| 137853322 | NM_003639.4(IKBKG): | CCAYATCAGGGGCCTGATACTGG | Incontinentia pigmenti |
| | c.1219A > G (p.Met407Val) | | syndrome |
| 387906267 | NM_000022.2(ADA): | CCCCYGGGAAGGGAAGAAAGGGG, | Severe combined |
| | c.219 − 2A > G | GCCCCYGGGAAGGGAAGAAAGGG, | immunodeficiency due to |
| | | AGCCCCYGGGAAGGGAAGAAAGG | ADA deficiency |
| 387906362 | NM_000492.3(CFTR): | TCAAATCYCACCCTCTGGCCAGG | Cystic fibrosis |
| | c.3717 + 4A > G | | |
| 397507442 | NM_002769.4(PRSS1): | CTTGYCATCATCATCAAAGGGGG, | Hereditary pancreatitis |
| | c.65A > G (p.Asp22Gly) | TCTTGYCATCATCATCAAAGGGG, | |
| | | ATCTTGYCATCATCATCAAAGGG, | |
| | | GATCTTGYCATCATCATCAAAGG | |
| 137853971 | NM_024598.3(USB1): | CCACCYGGTTTTCTCTTGATTGG | Poikiloderma with neutropenia |
| | c.502A > G (p.Arg168Gly) | | |
| 2228063 | NM_000067.2(CA2): | TGTYCTTCAGTGGCTGAGCTGGG, | |
| | c.754A > G (p.Asn252Asp) | CTGTYCTTCAGTGGCTGAGCTGG | |
| 387906743 | NM_001376.4(DYNC1H1): | ATTCAAGYAGATTACCTGATTGG | Spinal muscular atrophy, lower |
| | c.2909A > G (p.Tyr970Cys) | | extremity predominant 1, |
| | | | autosomal dominant |
| 387906772 | NM_002052.4(GATA4): | TCCGCAYTGCAAGAGGCCTGGGG, | Atrial septal defect 2 |
| | c.928A > G (p.Met310Val) | TTCCGCAYTGCAAGAGGCCTGGG | |
| 387906825 | NM_000414.3(HSD17B4): | TGCCACAYACTCTGGCTTCAGGG | Gonadal dysgenesis with |
| | c.650A > G (p.Tyr217Cys) | | auditory dysfunction, |
| | | | autosomal recessive |
| | | | inheritance |
| 387906895 | NM_006587.3(CORIN): | GGATAACYTGTACTGTTGTAGGG | Preeclampsia/eclampsia 5 |
| | c.1414A > G (p.Ser472Gly) | | |
| 387906957 | NM_016013.3(NDUFAF1): | ACCYTGACCTCCTGCCAGTAGGG, | Mitochondrial complex I |
| | c.758A > G (p.Lys253Arg) | TACCYTGACCTCCTGCCAGTAGG | deficiency |
| 28933682 | NM_000132.3(F8): | TAGCCAYTGATTGCTGGAGAAGG | Hereditary factor VIII |
| | c.5822A > G (p.Asn1941Ser) | | deficiency disease |
| 387907135 | NM_016464.4(TMEM138): | CAGYACAACACTGCTGCTGTGGG, | Joubert syndrome 16 |
| | c.389A > G (p.Tyr130Cys) | GCAGYACAACACTGCTGCTGTGG | |
| 137854530 | NM_001077488.3(GNAS): | GCCCAYGGCGGCGGCGGCGGCGG | Pseudohypoparathyroidism |
| | c.1A > G (p.Met1Val) | | type 1A |
| 387907176 | NM_018105.2(THAP1): | CCTCACTYGTGGAAAGAAACGGG | Dystonia 6, torsion |
| | c.70A > G (p.Lys24Glu) | | |
| 137854593 | NM_000397.3(CYBB): | TCACAYCTTTCTCCTCATCATGG | Chronic granulomatous |
| | c.1499A > G (p.Asp500Gly) | | disease, X-linked |
| 387907226 | NM_000076.2(CDKN1C): | CGCTYGGCGAAGAAATCTGCGGG, | Intrauterine growth retardation, |
| | c.832A > G (p.Lys278Glu) | GCGCTYGGCGAAGAAATCTGCGG | metaphyseal dysplasia, adrenal |
| | | | hypoplasia congenita, and |
| | | | genital anomalies |
| 387907242 | NM_022912.2(REEP1): | TCCYGTCAAAGGAAAAACAGAGG | Distal hereditary motor |
| | c.304 − 2A > G | | neuronopathy type 5B |
| 387907291 | NM_022787.3(NMNAT1): | TGTYTCTCTGCAAAGGGGCCAGG | Leber congenital amaurosis 9 |
| | c.817A > G (p.Asn273Asp) | | |
| 387907576 | NM_001287.5(CLCN7): | TGTCAYAGTCCAAGCTCTGCAGG | Osteopetrosis autosomal |
| | c.296A > G (p.Tyr99Cys) | | dominant type 2, Osteopetrosis |
| | | | autosomal recessive 4 |
| 730880531 | NM_000256.3(MYBPC3): | GTACCYGGGTGGGGGCCGCAGGG, | Familial hypertrophic |
| | c.1227 − 2A > G | TGTACCYGGGTGGGGGCCGCAGG | cardiomyopathy 4, |
| | | | Cardiomyopathy |
| 267606643 | NM_013411.4(AK2): | TCAYCTTTCATGGGCTCTTTTGG | Reticular dysgenesis |
| | c.494A > G (p.Asp165Gly) | | |
| 267606705 | NM_005188.3(CBL): | TATTTYACATAGTTGGAATGTGG | Noonan syndrome-like |
| | c.1144A > G (p.Lys382Glu) | | disorder with or without |
| | | | juvenile myelomonocytic |
| | | | leukemia |
| 62642934 | NM_000277.1(PAH): | GGCCAAYTTCCTGTAATTGGGGG, | Phenylketonuria, |
| | c.916A > G (p.Ile306Val) | AGGCCAAYTTCCTGTAATTGGGG | Hyperphenylalaninemia, non- |
| | | | pku |
| 267606782 | NM_000117.2(EMD): | TCCAYGGCGGGTGCGGGCTCAGG | Emery-Dreifuss muscular |
| | c.1A > G (p.Met1Val) | | dystrophy, X-linked |
| 267606820 | NM_014053.3(FLVCR1): | AGGCGTYGACCAGCGAGTACAGG | Posterior column ataxia with |
| | c.361A > G (p.Asn121Asp) | | retinitis pigmentosa |

In some embodiments, any of the base editors provided herein may be used to treat a disease or disorder. For example, any base editors provided herein may be used to correct one or more mutations associated with any of the diseases or disorders provided herein. Exemplary diseases or disorders that may be treated include, without limitation, 3-Methylglutaconic aciduria type 2, 46,XY gonadal dysgenesis, 4-Alpha-hydroxyphenylpyruvate hydroxylase deficiency, 6-pyruvoyl-tetrahydropterin synthase deficiency, achromatopsia, Acid-labile subunit deficiency, Acrodysostosis, acroerythrokeratoderma, ACTH resistance, ACTH-independent macronodular adrenal hyperplasia, Activated PI3K-delta syndrome, Acute intermittent porphyria, Acute myeloid leukemia, Adams-Oliver syndrome 1/5/6, Adenylosuccinate lyase deficiency, Adrenoleukodystrophy, Adult neuronal ceroid lipofuscinosis, Adult onset ataxia with oculomotor apraxia, Advanced sleep phase syndrome, Age-related macular degeneration, Alagille syndrome, Alexander disease, Allan-Herndon-Dudley syndrome, Alport syndrome, X-linked recessive, Alternating hemiplegia of childhood, Alveolar capillary dysplasia with misalignment of pulmonary veins, Amelogenesis imperfecta, Amyloidogenic transthyretin amyloidosis, Amyotrophic lateral sclerosis, Anemia (nonspherocytic hemolytic, due to G6PD deficiency), Anemia (sideroblastic, pyridoxine-refractory, autosomal recessive), Anonychia, Antithrombin III deficiency, Aortic aneurysm, Aplastic anemia, Apolipoprotein C2 deficiency, Apparent mineralocorticoid excess, Aromatase deficiency, Arrhythmogenic right ventricular cardiomyopathy, Familial hypertrophic cardiomyopathy, Hypertrophic cardiomyopathy, Arthrogryposis multiplex congenital, Aspartylglycosaminuria, Asphyxiating thoracic dystrophy, Ataxia with vitamin E deficiency, Ataxia (spastic), Atrial fibrillation, Atrial septal defect, atypical hemolytic-uremic syndrome, autosomal dominant CD11C+/CD1C+dendritic cell deficiency, Autosomal dominant progressive external ophthalmoplegia with mitochondrial DNA deletions, Baraitser-Winter syndrome, Bartter syndrome, Basa ganglia calcification, Beckwith-Wiedemann syndrome, Benign familial neonatal seizures, Benign scapuloperoneal muscular dystrophy, Bernard Soulier syndrome, Beta thalassemia intermedia, Beta-D-mannosidosis, Bietti crystalline corneoretinal dystrophy, Bile acid malabsorption, Biotinidase deficiency, Borjeson-Forssman-Lehmann syndrome, Boucher Neuhauser syndrome, Bowen-Conradi syndrome, Brachydactyly, Brown-Vialetto-Van laere syndrome, Brugada syndrome, Cardiac arrhythmia, Cardiofaciocutaneous syndrome, Cardiomyopathy, Carnevale syndrome, Carnitine palmitoyltransferase II deficiency, Carpenter syndrome, Cataract, Catecholaminergic polymorphic ventricular tachycardia, Central core disease, Centromeric instability of chromosomes 1,9 and 16 and immunodeficiency, Cerebral autosomal dominant arteriopathy, Cerebro-oculo-facio-skeletal syndrome, Ceroid lipofuscinosis, Charcot-Marie-Tooth disease, Cholestanol storage disease, Chondrocalcinosis, Chondrodysplasia, Chronic progressive multiple sclerosis, Coenzyme Q10 deficiency, Cohen syndrome, Combined deficiency of factor V and factor VIII, Combined immunodeficiency, Combined oxidative phosphorylation deficiency, Combined partial 17-alpha-hydroxylase/17,20-lyase deficiency, Complement factor d deficiency, Complete combined 17-alpha-hydroxylase/17,20-lyase deficiency, Cone-rod dystrophy, Congenital contractural arachnodactyly, Congenital disorder of glycosylation, Congenital lipomatous overgrowth, Neoplasm of ovary, PIK3CA Related Overgrowth Spectrum, Congenital long QT syndrome, Congenital muscular dystrophy, Congenital muscular hypertrophy-cerebral syndrome, Congenital myasthenic syndrome, Congenital myopathy with fiber type disproportion, Eichsfeld type congenital muscular dystrophy, Congenital stationary night blindness, Corneal dystrophy, Cornelia de Lange syndrome, Craniometaphyseal dysplasia, Crigler Najjar syndrome, Crouzon syndrome, Cutis laxa with osteodystrophy, Cyanosis, Cystic fibrosis, Cystinosis, Cytochrome-c oxidase deficiency, Mitochondrial complex I deficiency, D-2-hydroxyglutaric aciduria, Danon disease, Deafness with labyrinthine aplasia microtia and microdontia (LAMM), Deafness, Deficiency of acetyl-CoA acetyltransferase, Deficiency of ferroxidase, Deficiency of UDPglucose-hexose-1-phosphate uridylyltransferase, Dejerine-Sottas disease, Desbuquois syndrome, DFNA, Diabetes mellitus type 2, Diabetes-deafness syndrome, Diamond-Blackfan anemia, Diastrophic dysplasia, Dihydropteridine reductase deficiency, Dihydropyrimidinase deficiency, Dilated cardiomyopathy, Disseminated atypical mycobacterial infection, Distal arthrogryposis, Distal hereditary motor neuronopathy, Donnai Barrow syndrome, Duchenne muscular dystrophy, Becker muscular dystrophy, Dyschromatosis universalis hereditaria, Dyskeratosis congenital, Dystonia, Early infantile epileptic encephalopathy, Ehlers-Danlos syndrome, Eichsfeld type congenital muscular dystrophy, Emery-Dreifuss muscular dystrophy, Enamel-renal syndrome, Epidermolysis bullosa dystrophica inversa, Epidermolysis bullosa herpetiformis, Epilepsy, Episodic ataxia, Erythrokeratodermia variabilis, Erythropoietic protoporphyria, Exercise intolerance, Exudative vitreoretinopathy, Fabry disease, Factor V deficiency, Factor VII deficiency, Factor xiii deficiency, Familial adenomatous polyposis, breast cancer, ovarian cancer, cold urticarial, chronic infantile neurological, cutaneous and articular syndrome, hemiplegic migraine, hypercholesterolemia, hypertrophic cardiomyopathy, hypoalphalipoproteinemia, hypokalemia-hypomagnesemia, juvenile gout, hyperlipoproteinemia, visceral amyloidosis, hypophosphatemic vitamin D refractory rickets, FG syndrome, Fibrosis of extraocular muscles, Finnish congenital nephrotic syndrome, focal epilepsy, Focal segmental glomerulosclerosis, Frontonasal dysplasia, Frontotemporal dementia, Fructose-biphosphatase deficiency, Gamstorp-Wohlfart syndrome, Ganglioside sialidase deficiency, GATA-1-related thrombocytopenia, Gaucher disease, Giant axonal neuropathy, Glanzmann thrombasthenia, Glomerulocystic kidney disease, Glomerulopathy, Glucocorticoid resistance, Glucose-6-phosphate transport defect, Glutaric aciduria, Glycogen storage disease, Gorlin syndrome, Holoprosencephaly, GRACILE syndrome, Haemorrhagic telangiectasia, Hemochromatosis, Hemoglobin H disease, Hemolytic anemia, Hemophagocytic lymphohistiocytosis, Carcinoma of colon, Myhre syndrome, leukoencephalopathy, Hereditary factor IX deficiency disease, Hereditary factor VIII deficiency disease, Hereditary factor XI deficiency disease, Hereditary fructosuria, Hereditary Nonpolyposis Colorectal Neoplasm, Hereditary pancreatitis, Hereditary pyropoikilocytosis, Elliptocytosis, Heterotaxy, Heterotopia, Histiocytic medullary reticulosis, Histiocytosis-lymphadenopathy plus syndrome, HNSHA due to aldolase A deficiency, Holocarboxylase synthetase deficiency, Homocysteinemia, Howel-Evans syndrome, Hydatidiform mole, Hypercalciuric hypercalcemia, Hyperimmunoglobulin D, Mevalonic aciduria, Hyperinsulinemic hypoglycemia, Hyperkalemic Periodic Paralysis, Paramyotonia congenita of von Eulenburg, Hyperlipoproteinemia, Hypermanganesemia, Hypermethioninemia, Hyperphosphatasemia, Hypertension, hypomagnesemia, Hypobetalipoproteinemia, Hypocalcemia, Hypogonadotropic hypogonadism, Hypogonadotropic hypogonadism, Hypohidrotic ectodermal dysplasia, Hyper-IgM immunodeficiency, Hypohidrotic X-linked ectodermal dysplasia, Hypomagnesemia, Hypoparathyroidism, Idiopathic fibrosing alveolitis, Immunodeficiency, Immunoglobulin A deficiency, Infantile hypophosphatasia, Infantile Parkinsonism-dystonia, Insulin-dependent diabetes mellitus, Intermediate maple syrup urine disease, Ischiopatellar dysplasia, Islet cell hyperplasia, Isolated growth hormone deficiency, Isolated lutropin deficiency, Isovaleric acidemia, Joubert syndrome, Juvenile polyposis syndrome, Juvenile retinoschisis, Kallmann syndrome, Kartagener syndrome, Kugelberg-Welander disease, Lattice corneal dystrophy, Leber congenital amaurosis, Leber optic atrophy, Left ventricular noncompaction, Leigh disease, Mitochondrial complex I deficiency, Leprechaunism syndrome, Arthrogryposis, Anterior horn cell disease, Leukocyte adhesion deficiency, Leukodystrophy, Leukoencephalopathy, Ovarioleukodystrophy, L-ferritin deficiency, Li-Fraumeni syndrome, Limb-girdle muscular dystrophy-dystroglycanopathy, Loeys-Dietz syndrome, Long QT syndrome, Macrocephaly/autism syndrome, Macular corneal dystrophy, Macular dystrophy, Malignant hyperthermia susceptibility, Malignant tumor of prostate, Maple syrup urine disease, Marden Walker like syndrome, Marfan syndrome, Marie Unna hereditary hypotrichosis, Mast cell disease, Meconium ileus, Medium-chain acyl-coenzyme A dehydrogenase deficiency, Melnick-Fraser syndrome, Mental retardation, Merosin deficient congenital muscular dystrophy, Mesothelioma, Metachromatic leukodystrophy, Metaphyseal chondrodysplasia, Methemoglobinemia, methylmalonic aciduria, homocystinuria, Microcephaly, chorioretinopathy, lymphedema, Microphthalmia, Mild non-PKU hyperphenylalanemia, Mitchell-Riley syndrome, mitochondrial 3-hydroxy-3-methylglutaryl-CoA synthase deficiency, Mitochondrial complex I deficiency, Mitochondrial complex III deficiency, Mitochondrial myopathy, Mucolipidosis III, Mucopolysaccharidosis, Multiple sulfatase deficiency, Myasthenic syndrome, Mycobacterium tuberculosis, Myeloperoxidase deficiency, Myhre syndrome, Myoclonic epilepsy, Myofibrillar myopathy, Myoglobinuria, Myopathy, Myopia, Myotonia congenital, Navajo neurohepatopathy, Nemaline myopathy, Neoplasm of stomach, Nephrogenic diabetes insipidus, Nephronophthisis, Nephrotic syndrome, Neurofibromatosis, Neutral lipid storage disease, Niemann-Pick disease, Non-ketotic hyperglycinemia, Noonan syndrome, Noonan syndrome-like disorder, Norum disease, Macular degeneration, N-terminal acetyltransferase deficiency, Oculocutaneous albinism, Oculodentodigital dysplasia, Ohdo syndrome, Optic nerve aplasia, Ornithine carbamoyltransferase deficiency, Orofaciodigital syndrome, Osteogenesis imperfecta, Osteopetrosis, Ovarian dysgenesis, Pachyonychia, Palmoplantar keratoderma, nonepidermolytic, Papillon-Lef\c3\xa8vre syndrome, Haim-Munk syndrome, Periodontitis, Peeling skin syndrome, Pendred syndrome, Peroxisomal fatty acyl-coa reductase 1 disorder, Peroxisome biogenesis disorder, Pfeiffer syndrome, Phenylketonuria, Phenylketonuria, Hyperphenylalaninemia, non-PKU, Pituitary hormone deficiency, Pityriasis rubra pilaris, Polyarteritis nodosa, Polycystic kidney disease, Polycystic lipomembranous osteodysplasia, Polymicrogyria, Pontocerebellar hypoplasia, Porokeratosis, Posterior column ataxia, Primary erythromelalgia, hyperoxaluria, Progressive familial intrahepatic cholestasis, Progressive pseudorheumatoid dysplasia, Propionic acidemia, Pseudohermaphroditism, Pseudohypoaldosteronism, Pseudoxanthoma elasticum-like disorder, Purine-nucleoside phosphorylase deficiency, Pyridoxal 5-phosphate-dependent epilepsy, Renal dysplasia, retinal pigmentary dystrophy, cerebellar ataxia, skeletal dysplasia, Reticular dysgenesis, Retinitis pigmentosa, Usher syndrome, Retinoblastoma, Retinopathy, RRM2B-related mitochondrial disease, Rubinstein-Taybi syndrome, Schnyder crystalline corneal dystrophy, Sebaceous tumor, Severe congenital neutropenia, Severe myoclonic epilepsy in infancy, Severe X-linked myotubular myopathy, onychodysplasia, facial dysmorphism, hypotrichosis, Short-rib thoracic dysplasia, Sialic acid storage disease, Sialidosis, Sideroblastic anemia, Small fiber neuropathy, Smith-Magenis syndrome, Sorsby fundus dystrophy, Spastic ataxia, Spastic paraplegia, Spermatogenic failure, Spherocytosis, Sphingomyelin/cholesterol lipidosis, Spinocerebellar ataxia, Split-hand/foot malformation, Spondyloepimetaphyseal dysplasia, Platyspondylic lethal skeletal dysplasia, Squamous cell carcinoma of the head and neck, Stargardt disease, Sucrase-isomaltase deficiency, Sudden infant death syndrome, Supravalvar aortic stenosis, Surfactant metabolism dysfunction, Tangier disease, Tatton-Brown-rahman syndrome, Thoracic aortic aneurysms and aortic dissections, Thrombophilia, Thyroid hormone resistance, TNF receptor-associated periodic fever syndrome (TRAPS), Tooth agenesis, Torsades de pointes, Transposition of great arteries, Treacher Collins syndrome, Tuberous sclerosis syndrome, Tyrosinase-negative oculocutaneous albinism, Tyrosinase-positive oculocutaneous albinism, Tyrosinemia, UDPglucose-4-epimerase deficiency, Ullrich congenital muscular dystrophy, Bethlem myopathy Usher syndrome, UV-sensitive syndrome, Van der Woude syndrome, popliteal pterygium syndrome, Very long chain acyl-CoA dehydrogenase deficiency, Vesicoureteral reflux, Vitreoretinochoroidopathy, Von Hippel-Lindau syndrome, von Willebrand disease, Waardenburg syndrome, Warsaw breakage syndrome, WFS1-Related Disorders, Wilson disease, Xeroderma pigmentosum, X-linked agammaglobulinemia, X-linked hereditary motor and sensory neuropathy, X-linked severe combined immunodeficiency, and Zellweger syndrome.

The development of nucleobase editing advances both the scope and effectiveness of genome editing. The nucleobase editors described here offer researchers a choice of editing with virtually no indel formation (NBE2), or more efficient editing with a low frequency (here, typically ≤1%) of indel formation (NBE3). That the product of base editing is, by definition, no longer a substrate likely contributes to editing efficiency by preventing subsequent product transformation, which can hamper traditional Cas9 applications. By removing the reliance on double-stranded DNA cleavage and stochastic DNA repair processes that vary greatly by cell state and cell type, nucleobase editing has the potential to expand the type of genome modifications that can be cleanly installed, the efficiency of these modifications, and the type of cells that are amenable to editing. It is likely that recent engineered Cas9 variants^{69,70,82} or delivery methods⁷¹ with improved DNA specificity, as well as Cas9 variants with altered PAM specificities,⁷² can be integrated into this strategy to provide additional nucleobase editors with improved DNA specificity or that can target an even wider range of disease-associated mutations. These findings also suggest that engineering additional fusions of dCas9 with enzymes that catalyze additional nucleobase transformations will increase the fraction of the possible DNA base changes that can be made through nucleobase editing. These results also suggest architectures for the fusion of other DNA-modifying enzymes, including methylases and demathylases, that mau enable additional types of programmable genome and epigenome base editing.

### Materials and Methods

Cloning. DNA sequences of all constructs and primers used in this paper are listed in the Supplementary Sequences. Plasmids containing genes encoding NBE1, NBE2, and NBE3 will be available from Addgene. PCR was performed using VeraSeq ULtra DNA polymerase (Enzymatics), or Q5 Hot Start High-Fidelity DNA Polymerase (New England Biolabs). NBE plasmids were constructed using USER cloning (New England Biolabs). Deaminase genes were synthesized as gBlocks Gene Fragments (Integrated DNA Technologies), and Cas9 genes were obtained from previously reported plasmids.¹⁸ Deaminase and fusion genes were cloned into pCMV (mammalian codon-optimized) or pET28b (E. coli codon-optimized) backbones. sgRNA expression plasmids were constructed using site-directed mutagenesis. Briefly, the primers listed in the Supplementary Sequences were 5′ phosphorylated using T4 Polynucleotide Kinase (New England Biolabs) according to the manufacturer's instructions. Next, PCR was performed using Q5 Hot Start High-Fidelity Polymerase (New England Biolabs) with the phosphorylated primers and the plasmid pFYF1320 (EGFP sgRNA expression plasmid) as a template according to the manufacturer's instructions. PCR products were incubated with DpnI (20 U, New England Biolabs) at 37° C. for 1 h, purified on a QIAprep spin column (Qiagen), and ligated using QuickLigase (New England Biolabs) according to the manufacturer's instructions. DNA vector amplification was carried out using Mach1 competent cells (ThermoFisher Scientific).

In vitro deaminase assay on ssDNA. Sequences of all ssDNA substrates are listed in the Supplementary Sequences. All Cy3-labelled substrates were obtained from Integrated DNA Technologies (IDT). Deaminases were expressed in vitro using the TNT T7 Quick Coupled Transcription/Translation Kit (Promega) according to the manufacturer's instructions using 1 g of plasmid. Following protein expression, 5 μL of lysate was combined with 35 μL of ssDNA (1.8 μM) and USER enzyme (1 unit) in CutSmart buffer (New England Biolabs) (50 mM potassium acetate, 29 mM Trisacetate, 10 mM magnesium acetate, 100 μg/mL BSA, pH 7.9) and incubated at 37° C. for 2 h. Cleaved U-containing substrates were resolved from full-length unmodified substrates on a 10% TBE-urea gel (Bio-Rad).

Expression and purification of His6-rAPOBEC1-linker-dCas9 fusions. E. Coli BL21 STAR (DE3)-competent cells (ThermoFisher Scientific) were transformed with plasmids encoding pET28b-His₆-rAPOBEC-linker-dCas9 with GGS, (GGS)₃, (SEQ ID NO: 596) XTEN, or (GGS)₇ (SEQ ID NO: 597) linkers. The resulting expression strains were grown overnight in Luria-Bertani (LB) broth containing 100 g/mL of kanamycin at 37° C. The cells were diluted 1:100 into the same growth medium and grown at 37° C. to OD₆₀₀=−0.6. The culture was cooled to 4° C. over a period of 2 h, and isopropyl-β-D-1-thiogalactopyranoside (IPTG) was added at 0.5 mM to induce protein expression. After ˜16 h, the cells were collected by centrifugation at 4,000 g and resuspended in lysis buffer (50 mM tris(hydroxymethyl)-aminomethane (Tris)-HCl, pH 7.0, 1 M NaCl, 20% glycerol, 10 mM tris(2.-carboxyethyl)phosphine (TCEP, Soltec Ventures)). The cells were lysed by sonication (20 s pulse-on. 20 s pulse-off for 8 min total at 6 W output) and the lysate supernatant was isolated following centrifugation at 25,000 g for 15 min. The lysate was incubated with His-Pur nickel-nitriloacetic acid (nickel-NTA) resin (ThermoFisher Scientific) at 4° C. for 1 h to capture the His-tagged fusion protein. The resin was transferred to a column and washed with 40 mL of lysis buffer. The His-tagged fusion protein was eluted in lysis buffer supplemented with 285 mM imidazole, and concentrated by ultrafiltration (Amicon-Millipore, 100-kDa molecular weight cut-off) to 1 mL total volume. The protein was diluted to 20 mL in low-salt purification buffer containing 50 mM tris(hydroxymethyl)aminomethane (Tris)-HCl pH 7.0, 0.1 M NaCl, 20% glycerol, 10 mM TCEP and loaded onto SP Sepharose Fast Flow resin (GE Life Sciences), The resin was washed with 40 mL of this low-salt buffer, and the protein eluted with 5 mL of activity buffer containing 50 mM tris(hydroxymethil)-aminomethane (Tris)-HCl, pH 7.0, 0.5 M NaCl, 20% glycerol, 10 mM TCEP. The eluted proteins were quantified on a SDSPAGE gel.

In vitro transcription of sgRNAs. Linear DNA fragments containing the T7 promoter followed by the 20-bp sgRNA target. sequence were transcribed in vitro using the primers listed in the Supplementary Sequences with the TranscriptAid T7 High Yield Transcription Kit (ThermoFisher Scientific) according to the manufacturer's instructions. sgRNA products were purified using the MEGAclear Kit (ThermoFisher Scientific) according to the manufacturer's instructions and quantified by UV absorbance.

Preparation of Cy3-conjugated dsDNA substrates. Sequences of 80-nucleotide unlabeled strands are listed in the Supplementary Sequences and were ordered as PAGE-purified oligonucleotides from IDT. The 25-nt Cy3-labeled primer listed in the Supplementary Sequences is complementary to the 3′ end of each 80-nt substrate. This primer was ordered as an HPLC-purified oligonucleotide from IDT. To generate the Cy3-labeled dsDNA substrates, the 80-nt strands (5 μL of a 100 μM solution) were combined with the Cy3-labeled primer (5 μL of a 100 μM solution) in NEBuffer 2 (38.25 μL of a 50 mM NaCl, 10 mMTris-HCl, 10 mM MgCl₂, 1 mM DTT, pH 7.9 solution, New England Biolabs) with dNTPs (0.75 μL of a 100 mM solution) and heated to 95° C. for 5 min, followed by a gradual cooling to 45° C. at a rate of 0.1° C./s. After this annealing period, Klenow exo (5 U, New England Biolabs) was added and the reaction was incubated at 37° C. for 1 h. The solution was diluted with Buffer PB (250 μL, Qiagen) and isopropanol (50 μL) and purified on a QIAprep spin column (Qiagen), eluting with 50 μL of Tris buffer.

Deaminase assay on dsDNA. The purified fusion protein (20 μL of 1.9 μM in activity buffer) was combined with 1 equivalent of appropriate sgRNA and incubated at ambient temperature for 5 min. The Cy3-labeled dsDNA substrate was added to final concentration of 125 nM and the resulting solution was incubated at 37° C. for 2 h. The dsDNA was separated from the fusion by the addition of Buffer PB (100 μL, Qiagen) and isopropanol (25 μL) and purified on a EconoSpin micro spin column (Epoch Life Science), eluting with 20 μL of CutSmart buffer (New England Biolabs). USER enzyme (1 U, New England Biolabs) was added to the purified, edited dsDNA and incubated at 37° C. for 1 h. The Cy3-labeled strand was fully denatured from its complement by combining 5 μL of the reaction solution with 15 μL of a DMSO-based loading buffer (5 mM Tris, 0.5 mM EDTA, 12.5% glycerol, 0.02% bromophenol blue, 0.02% xylene cyan, 80% DMSO). The full-length C-containing substrate was separated from any cleaved, U-containing edited substrates on a 10% TBE-urea gel (Bio-Rad) and imaged on a GE Amersham Typhoon imager.

Preparation of in vitro-edited dsDNA for high-throughput sequencing (HTS). The oligonucleotides listed in the Supplementary Sequences were obtained from IDT. Complementary sequences were combined (5 μL of a 100 μM solution) in Tris buffer and annealed by heating to 95° C. for 5 min, followed by a gradual cooling to 45° C. at a rate of 0.1° C./s to generate 60-bp dsDNA substrates. Purified fusion protein (20 μL of 1.9 μM in activity buffer) was combined with 1 equivalent of appropriate sgRNA and incubated at ambient temperature for 5 min. The 60-mer dsDNA substrate was added to final concentration of 125 nM and the resulting solution was incubated at 37° C. for 2 h. The dsDNA was separated from the fusion by the addition of Buffer PB (100 μL, Qiagen) and isopropanol (25 μL) and purified on a EconoSpin micro spin column (Epoch Life Science), eluting with 20 μL of Tris buffer. The resulting edited DNA (1 μL was used as a template) was amplified by PCR using the HTS primer pairs specified in the Supplementary Sequences and VeraSeq Ultra (Enzymatics) according to the manufacturer's instructions with 13 cycles of amplification. PCR reaction products were purified using RapidTips (Diffinity Genomics), and the purified DNA was amplified by PCR with primers containing sequencing adapters, purified, and sequenced on a MiSeq high-throughput DNA sequencer (Illumina) as previously described.⁷³

Cell culture. HEK293T (ATCC CRL-3216), U2OS (ATCC-HTB-96) and ST486 cells (ATCC) were maintained in Dulbecco's Modified Eagle's Medium plus GlutaMax (ThermoFisher) supplemented with 10% (v/v) fetal bovine serum (FBS) and penicillin/streptomycin (lx, Amresco), at 37° C. with 5% CO₂. HCC1954 cells (ATCC CRL-2338) were maintained in RPMI-1640 medium (ThermoFisher Scientific) supplemented as described above. Immortalized rat astrocytes containing the ApoE4 isoform of the APOE gene (Taconic Biosciences) were cultured in Dulbecco's Modified Eagle's Medium plus GlutaMax (ThermoFisher Scientific) supplemented with 10% (v/v) fetal bovine serum (FBS) and 200 g/mL Geneticin (ThermoFisher Scientific).

Transfections. HEK293T cells were seeded on 48-well collagen-coated BioCoat plates (Corning) and transfected at approximately 85% confluency. Briefly, 750 ng of NBE and 250 ng of sgRNA expression plasmids were transfected using 1.5 μl of Lipofectamine 2000 (ThermoFisher Scientific) per well according to the manufacturer's protocol. Astrocytes, U2OS, HCC1954, HEK293T and ST486 cells were transfected using appropriate AMAXA NUCLEOFECTOR™ II programs according to manufacturer's instructions. 40 ng of infrared RFP (Addgene plasmid 45457)⁷⁴ was added to the nucleofection solution to assess nucleofection efficiencies in these cell lines. For astrocytes, U2OS, and ST486 cells, nucleofection efficiencies were 25%, 74%, and 92%, respectively. For HCC1954 cells, nucleofection efficiency was <10%. Therefore, following trypsinization, the HCC1954 cells were filtered through a 40 micron strainer (Fisher Scientific), and the nucleofected HCC1954 cells were collected on a Beckman Coulter MoFlo XDP Cell Sorter using the iRFP signal (abs 643 nm, em 670 nm). The other cells were used without enrichment of nucleofected cells.

High-throughput DNA sequencing of genomic DNA samples. Transfected cells were harvested after 3 d and the genomic DNA was isolated using the Agencourt DNAdvance Genomic DNA Isolation Kit (Beckman Coulter) according to the manufacturer's instructions. On-target and off-target genomic regions of interest were amplified by PCR with flanking HTS primer pairs listed in the Supplementary Sequences. PCR amplification was carried out with Phusion high-fidelity DNA polymerase (ThermoFisher) according to the manufacturer's instructions using 5 ng of genomic DNA as a template. Cycle numbers were determined separately for each primer pair as to ensure the reaction was stopped in the linear range of amplification (30, 28, 28, 28, 32, and 32 cycles for EMX1, FANCF, HEK293 site 2, HEK293 site 3, HEK293 site 4, and RNF2 primers, respectively). PCR products were purified using RapidTips (Diffinity Genomics). Purified DNA was amplified by PCR with primers containing sequencing adaptors. The products were gel-purified and quantified using the QUANT-IT™ PicoGreen dsDNA Assay Kit (ThermoFisher) and KAPA Library Quantification Kit-Illumina (KAPA Biosystems). Samples were sequenced on an Illumina MiSeq as previously described.⁷³

Data analysis. Sequencing reads were automatically demultiplexed using MiSeq Reporter (Illumina), and individual FASTQ files were analyzed with a custom Matlab script provided in the Supplementary Notes. Each read was pairwise aligned to the appropriate reference sequence using the Smith-Waterman algorithm. Base calls with a Q-score below 31 were replaced with N's and were thus excluded in calculating nucleotide frequencies. This treatment yields an expected MiSeq base-calling error rate of approximately 1 in 1,000. Aligned sequences in which the read and reference sequence contained no gaps were stored in an alignment table from which base frequencies could be tabulated for each locus.

Indel frequencies were quantified with a custom Matlab script shown in the Supplementary Notes using previously described criteria⁷¹. Sequencing reads were scanned for exact matches to two 10-bp sequences that flank both sides of a window in which indels might occur. If no exact matches were located, the read was excluded from analysis. If the length of this indel window exactly matched the reference sequence the read was classified as not containing an indel. If the indel window was two or more bases longer or shorter than the reference sequence, then the sequencing read was classified as an insertion or deletion, respectively.

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### Supplementary Sequences

Primers used for generating sgRNA transfection plasmids. rev_sgRNA_plasmid was used in all cases. The pFYF1320 plasmid was used as template as noted in Materials and Methods section. SEQ ID NOs: 329-338 appear from top to bottom below, respectively.

| | |
|---|---|
| rev_sgRNA_plasmid | GGTGTTTCGTCCTTTCCACAAG |
| fwd _p53 _Y163C | GCTTGCAGATGGCCATGGCGGTTTT |
| | AGAGCTAGAAATAGCAAGTTAAAAT |
| | AAGGC |
| fwd_p53_N239D | TGTCACACATGTAGTTGTAGGTTTT |
| | AGAGCTAGAAATAGCAAGTTAAAAT |
| | AAGGC |
| fwd_APOE4_C158R | GAAGCGCCTGGCAGTGTACCGTTTT |
| | AGAGCTAGAAATAGCAAGTTAAAAT |
| | AAGGC |
| fwd_EMX1 | GAGTCCGAGCAGAAGAAGAAGTTTT |
| | AGAGCTAGAAATAGCAAGTTAAAAT |
| | AAGGC |
| fwd_FANCF | GGAATCCCTTCTGCAGCACCGTTTT |
| | AGAGCTAGAAATAGCAAGTTAAAAT |
| | AAGGC |
| fwd_HEK293_2 | GAACACAAAGCATAGACTGCGTTTT |
| | AGAGCTAGAAATAGCAAGTTAAAAT |
| | AAGGC |
| fwd_HEK293_3 | GGCCCAGACTGAGCACGTGAGTTTT |
| | AGAGCTAGAAATAGCAAGTTAAAAT |
| | AAGGC |
| fwd_HEK293_4 | GGCACTGCGGCTGGAGGTGGGTTTT |
| | AGAGCTAGAAATAGCAAGTTAAAAT |
| | AAGGC |
| fwd_RNF2 | GTCATCTTAGTCATTACCTGGTTTT |
| | AGAGCTAGAAATAGCAAGTTAAAAT |
| | AAGGC |

Sequences of all ssDNA substrates used in in vitro deaminase assays. SEQ ID NOs: 339-341 appear from top to bottom below, respectively.

| | |
|---|---|
| rAPOBEC1 substrate | Cy3-ATTATTATTATTCCGCGGA |
| | TTTATTTATTTATTTATTTATTT |
| hAID/pmCDA1 substrate | Cy3-ATTATTATTATTAGCTATT |
| | TATTTATTTATTTATTTATTT |
| hAPOBEC3G substrate | Cy3-ATTATTATTATTCCCGGAT |
| | TTATTTATTTATTTATTTATTT |

Primers used for generating PCR products to serve as substrates for T7 transcription of sgRNAs for gel-based deaminase assay. rev_gRNA_T7 was used in all cases. The pFYF1320 plasmid was used as template as noted in Materials and Methods section. SEQ ID NOs: 342-365 appear from top to bottom below, respectively.

| | |
|---|---|
| rev_sgRNA_T7 | AAAAAAAGCACCGACTCGGTG |
| fwd_sgRNA_T7_dsDNA_2 | TAATACGACTCACTATAGGCCGCGG |
| | ATTTATTTATTTAAGTTTTAGAGCT |
| | AGAAATAGCA |
| fwd_sgRNA_T7_dsDNA_3 | TAATACGACTCACTATAGGTCCGCG |
| | GATTTATTTATTTAGTTTTAGAGCT |
| | AGAAATAGCA |
| fwd_sgRNA_T7_dsDNA_4 | TAATACGACTCACTATAGGTTCCGC |
| | GGATTTATTTATTAGTTTTAGAGCT |
| | AGAAATAGCA |
| fwd_sgRNA_T7_dsDNA_5 | TAATACGACTCACTATAGGATTCCG |
| | CGGATTTATTTATTGTTTTAGAGCT |
| | AGAAATAGCA |
| fwd_sgRNA_T7_dsDNA_6 | TAATACGACTCACTATAGGTATTCC |
| | GCGGATTTATTTATGTTTTAGAGCT |
| | AGAAATAGCA |
| fwd_sgRNA_T7_dsDNA_7 | TAATACGACTCACTATAGGTTATTC |
| | CGCGGATTTATTTAGTTTTAGAGCT |
| | AGAAATAGCA |
| fwd_sgRNA_T7_dsDNA_8 | TAATACGACTCACTATAGGATTATT |
| | CCGCGGATTTATTTGTTTTAGAGCT |
| | AGAAATAGCA |
| fwd_sgRNA_T7_dsDNA_9 | TAATACGACTCACTATAGGTATTAT |
| | TCCGCGGATTTATTGTTTTAGAGCT |
| | AGAAATAGCA |
| fwd_sgRNA_T7_dsDNA_10 | TAATACGACTCACTATAGGATTATT |
| | ATCCGCGGATTTATGTTTTAGAGCT |
| | AGAAATAGCA |
| fwd_sgRNA_T7_dsDNA_11 | TAATACGACTCACTATAGGTATTAT |
| | ATTCCGCGGATTTAGTTTTAGAGCT |
| | AGAAATAGCA |
| fwd_sgRNA_T7_dsDNA_12 | TAATACGACTCACTATAGGTTATTA |
| | TATTCCGCGGATTTGTTTTAGAGCT |
| | AGAAATAGCA |
| fwd_sgRNA_T7_dsDNA_13 | TAATACGACTCACTATAGGATTATT |
| | ATATTCCGCGGATTGTTTTAGAGCT |
| | AGAAATAGCA |
| fwd_sgRNA_T7_dsDNA_14 | TAATACGACTCACTATAGGTATTAT |
| | TATATTCCGCGGATGTTTTAGAGCT |
| | AGAAATAGCA |
| fwd_sgRNA_T7_dsDNA_15 | TAATACGACTCACTATAGGATTATT |
| | ATTATTACCGCGGAGTTTTAGAGCT |
| | AGAAATAGCA |
| fwd_sgRNA_T7_dsDNA_18 | TAATACGACTCACTATAGGATTATT |
| | ATTATTATTACCGCGTTTTAGAGCT |
| | AGAAATAGCA |
| fwd_sgRNA_T7_dsDNA | TAATACGACTCACTATAGGGGAGGA |
| APOE4_C112R | CGTGCGCGGCCGCCGTTTTAGAGCT |
| | AGAAATAGCA |
| fwd_sgRNA_T7_dsDNA | TAATACGACTCACTATAGGGAAGCG |
| APOE4_C158R | CCTGGCAGTGTACCGTTTTAGAGCT |
| | AGAAATAGCA |
| fwd_sgRNA_T7_dsDNA_ | TAATACGACTCACTATAGGCTGTGG |
| CTNNB1_T41A | CAGTGGCACCAGAAGTTTTAGAGCT |
| | AGAAATAGCA |
| fwd_sgRNA_T7_dsDNA_ | TAATACGACTCACTATAGGCCTCCC |
| HRAS_Q61R | GGCCGGCGGTATCCGTTTTAGAGCT |
| | AGAAATAGCA |
| fwd_sgRNA_T7_dsDNA_ | TAATACGACTCACTATAGGGCTTGC |
| 53_Y163C | AGATGGCCATGGCGGTTTTAGAGCT |
| | AGAAATAGCA |
| fwd_sgRNA_T7_dsDNA_ | TAATACGACTCACTATAGGACACAT |
| 53_Y236C | GCAGTTGTAGTGGAGTTTTAGAGCT |
| | AGAAATAGCA |
| fwd_sgRNA_T7_dsDNA_ | TAATACGACTCACTATAGGTGTCAC |
| 53_N23_9D | ACATGTAGTTGTAGGTTTTAGAGCT |
| | AGAAATAGCA |

Sequences of 80-nucleotide unlabeled strands and Cy3-labeled universal primer used in gel-based dsDNA deaminase assays. SEQ ID NOs: 366-390 appear from top to bottom below, respectively.

| | |
|---|---|
| Cy3-primer | Cy3-GTAGGTAGTTAGGATGAATGGAAGGTTGGTA |
| dsDNA_2 | GTCCATGGATCCAGAGGTCATCCATTAAATAAATA |
| | AATCCGCGGGGCTATACCAACCTTCCATTCATCCT |
| | AACTACCTAC |
| dsDNA_3 | GTCCATGGATCCAGAGGTCATCCATAAATAAATAA |
| | ATCCGCGGAAGCTATACCAACCTTCCATTCATCCT |
| | AACTACCTAC |
| dsDNA_4 | GTCCATGGATCCAGAGGTCATCCATAATAAATAAA |
| | TCCGCGGAAGGCTATACCAACCTTCCATTCATCCT |
| | AACTACCTAC |
| dsDNA_5 | GTCCATGGATCCAGAGGTCATCCAAATAAATAAAT |
| | CCGCGGAATGGCTATACCAACCTTCCATTCATCCT |
| | AACTACCTAC |
| dsDNA_6 | GTCCATGGATCCAGAGGTCATCCAATAAATAAATC |
| | CGCGGAATAGGCTATACCAACCTTCCATTCATCCT |
| | AACTACCTAC |
| dsDNA_7 | GTCCATGGATCCAGAGGTCATCCATAAATAAATCC |
| | GCGGAATAAGGCTATACCAACCTTCCATTCATCCT |
| | AACTACCTAC |
| dsDNA_8 | GTCCATGGATCCAGAGGTCATCCAAAATAAATCCG |
| | CGGAATAATGGCTATACCAACCTTCCATTCATCCT |
| | AACTACCTAC |
| dsDNA_9 | GTCCATGGATCCAGAGGTCATCCAAATAAATCCGC |
| | GGAATAATAGGCTATACCAACCTTCCATTCATCCT |
| | AACTACCTAC |
| dsDNA_10 | GTCCATGGATCCAGAGGTCATCCAATAAATCCGCG |
| | GATAATAATGGCTATACCAACCTTCCATTCATCCT |
| | AACTACCTAC |
| dsDNA_11 | GTCCATGGATCCAGAGGTCATCCATAAATCCGCGG |
| | AATATAATAGGCTATACCAACCTTCCATTCATCCT |
| | AACTACCTAC |
| dsDNA_12 | GTCCATGGATCCAGAGGTCATCCAAAATCCGCGGA |
| | ATATAATAAGGCTATACCAACCTTCCATTCATCCT |
| | AACTACCTAC |
| dsDNA_13 | GTCCATGGATCCAGAGGTCATCCAAATCCGCGGAA |
| | TATAATAATGGCTATACCAACCTTCCATTCATCCT |
| | AACTACCTAC |
| dsDNA_14 | GTCCATGGATCCAGAGGTCATCCAATCCGCGGAAT |
| | ATAATAATAGGCTATACCAACCTTCCATTCATCCT |
| | AACTACCTAC |
| dsDNA_15 | GTCCATGGATCCAGAGGTCATCCATCCGCGGTAAT |
| | AATAATAATGGCTATACCAACCTTCCATTCATCCT |
| | AACTACCTAC |
| dsDNA_18 | GTCCATGGATCCAGAGGTCATCCAGCGGTAATAAT |
| | AATAATAATGGCTATACCAACCTTCCATTCATCCT |
| | AACTACCTAC |
| dsDNA_noC | GTCCATGGATCCAGAGGTCATCCATTAAATAAATA |
| | AATTAATATTACTATACCAACCTTCCATTCATCCT |
| | AACTACCTAC |
| dsDNA_8U | 5Cy3- |
| | GTAGGTAGTTAGGATGAATGGAAGGTTGGTGTAGA |
| | TTATTATCUGCGGATTTATTGGATGACCTCTGGAT |
| | CCATGGACAT |
| dsDNA_APOE_ | GCACCTCGCCGCGGTACTGCACCAGGCGGCCGCGC |
| C112R | ACGTCCTCCATGTCTACCAACCTTCCATTCATCCT |
| | AACTACCTAC |
| dsDNA_APOE_ | CGGCGCCCTCGCGGGCCCCGGCCTGGTACACTGCC |
| C158R | AGGCGCTTCTGCAGTACCAACCTTCCATTCATCCT |
| | AACTACCTAC |
| dsDNA_CTNN | GTCTTACCTGGACTCTGGAATCCATTCTGGTGCCA |
| B1_T41A | CTGCCACAGCTCCTTACCAACCTTCCATTCATCCT |
| | AACTACCTAC |
| dsDNA_HRAS_ | GGAGACGTGCCTGTTGGACATCCTGGATACCGCCG |
| Q61R | GCCGGGAGGAGTACTACCAACCTTCCATTCATCCT |
| | AACTACCTAC |
| dsDNA_p53_ | ACCCCCGCCCGGCACCCGCGTCCGCGCCATGGCCA |
| Y163C | TCTGCAAGCAGTCATACCAACCTTCCATTCATCCT |
| | AACTACCTAC |
| dsDNA_p53_Y | AGGTTGGCTCTGACTGTACCACCATCCACTACAAC |
| 236C | TGCATGTGTAACAGTACCAACCTTCCATTCATCCT |
| | AACTACCTAC |
| dsDNA_p53_N | TGGCTCTGACTGTACCACCATCCACTACAACTACA |
| 239D | TGTGTGACAGTTCCTACCAACCTTCCATTCATCCT |
| | AACTACCTAC |

Primers used for generating PCR products to serve as substrates for T7 transcription of sgRNAs for high-throughput sequencing. rev_gRNA_T7 (above) was used in all cases. The pFYF1320 plasmid was used as template as noted in Materials and Methods section. SEQ ID NOs: 391-442 appear from top to bottom below, respectively.

| | |
|---|---|
| fwd_sgRNA_T7_ | TAATACGACTCACTATAGGTTATTTCGTGGA |
| HTS_ base | TTTATTTAGTTTTAGAGCTAGAAATAGCA |
| fwd_sgRNA_T7_ | TAATACGACTCACTATAGGATATTTCGTGGA |
| HTS_1A | TTTATTTAGTTTTAGAGCTAGAAATAGCA |
| fwd_sgRNA_T7_ | TAATACGACTCACTATAGGCTATTTCGTGGA |
| HTS_1C | TTTATTTAGTTTTAGAGCTAGAAATAGCA |
| fwd_sgRNA_T7_ | TAATACGACTCACTATAGGGTATTTCGTGGA |
| HTS_1G | TTTATTTAGTTTTAGAGCTAGAAATAGCA |
| fwd_sgRNA_T7_ | TAATACGACTCACTATAGGTAATTTCGTGGA |
| HTS_2A | TTTATTTAGTTTTAGAGCTAGAAATAGCA |
| fwd_sgRNA_T7_ | TAATACGACTCACTATAGGTCATTTCGTGGA |
| HTS_2C | TTTATTTAGTTTTAGAGCTAGAAATAGCA |
| fwd_sgRNA_T7_ | TAATACGACTCACTATAGGTGATTTCGTGGA |
| HTS_2G | TTTATTTAGTTTTAGAGCTAGAAATAGCA |
| fwd_sgRNA_T7_ | TAATACGACTCACTATAGGTTTTTTCGTGGA |
| HTS_3T | TTTATTTAGTTTTAGAGCTAGAAATAGCA |
| fwd_sgRNA_T7_ | TAATACGACTCACTATAGGTTCTTTCGTGGA |
| HTS_3C | TTTATTTAGTTTTAGAGCTAGAAATAGCA |
| fwd_sgRNA_T7_ | TAATACGACTCACTATAGGTTGTTTCGTGGA |
| HTS_3G | TTTATTTAGTTTTAGAGCTAGAAATAGCA |
| fwd_sgRNA_T7_ | TAATACGACTCACTATAGGTTAATTCGTGGA |
| HTS_4A | TTTATTTAGTTTTAGAGCTAGAAATAGCA |
| fwd_sgRNA_T7_ | TAATACGACTCACTATAGGTTACTTCGTGGA |
| HTS_4C | TTTATTTAGTTTTAGAGCTAGAAATAGCA |
| fwd_sgRNA_T7_ | TAATACGACTCACTATAGGTTAGTTCGTGGA |
| HTS_4G | TTTATTTAGTTTTAGAGCTAGAAATAGCA |
| fwd_sgRNA_T7_ | TAATACGACTCACTATAGGTTATATCGTGGA |
| HTS_5A | TTTATTTAGTTTTAGAGCTAGAAATAGCA |
| fwd_sgRNA_T7_ | TAATACGACTCACTATAGGTTATCTCGTGGA |
| HTS_5C | TTTATTTAGTTTTAGAGCTAGAAATAGCA |
| fwd_sgRNA_T7_ | TAATACGACTCACTATAGGTTATGTCGTGGA |
| HTS_5G | TTTATTTAGTTTTAGAGCTAGAAATAGCA |
| fwd_sgRNA_T7_ | TAATACGACTCACTATAGGTTATTACGTGGA |
| HTS_6A | TTTATTTAGTTTTAGAGCTAGAAATAGCA |
| fwd_sgRNA_T7_ | TAATACGACTCACTATAGGTTATTCCGTGGA |
| HTS_6C | TTTATTTAGTTTTAGAGCTAGAAATAGCA |
| fwd_sgRNA_T7_ | TAATACGACTCACTATAGGTTATTGCGTGGA |
| HTS_6G | TTTATTTAGTTTTAGAGCTAGAAATAGCA |
| fwd_sgRNA_T7_ | TAATACGACTCACTATAGGTTATTTCATGGA |
| HTS_8A | TTTATTTAGTTTTAGAGCTAGAAATAGCA |
| fwd_sgRNA_T7_ | TAATACGACTCACTATAGGTTATTTCTTGGA |
| HTS_8T | TTTATTTAGTTTTAGAGCTAGAAATAGCA |
| fwd_sgRNA_T7_ | TAATACGACTCACTATAGGTTATTTCCTGGA |
| HTS_8C | TTTATTTAGTTTTAGAGCTAGAAATAGCA |
| fwd_sgRNA_T7_ | TAATACGACTCACTATAGGTTATTTCGAGGA |
| HTS_9A | TTTATTTAGTTTTAGAGCTAGAAATAGCA |
| fwd_sgRNA_T7_ | TAATACGACTCACTATAGGTTATTTCGCGGA |
| HTS_9C | TTTATTTAGTTTTAGAGCTAGAAATAGCA |
| fwd_sgRNA_T7_ | TAATACGACTCACTATAGGTTATTTCGGGGA |
| HTS_9G | TTTATTTAGTTTTAGAGCTAGAAATAGCA |
| fwd_sgRNA_T7_ | TAATACGACTCACTATAGGTTATTTCGTAGA |
| HTS_10A | TTTATTTAGTTTTAGAGCTAGAAATAGCA |
| fwd_sgRNA_T7_ | TAATACGACTCACTATAGGTTATTTCGTTGA |
| HTS_10T | TTTATTTAGTTTTAGAGCTAGAAATAGCA |
| fwd_sgRNA_T7_ | TAATACGACTCACTATAGGTTATTTCGTCGA |
| HTS_10C | TTTATTTAGTTTTAGAGCTAGAAATAGCA |
| fwd_sgRNA_T7_ | TAATACGACTCACTATAGGTTATTTCGTGAA |
| HTS_11A | TTTATTTAGTTTTAGAGCTAGAAATAGCA |
| fwd_sgRNA_T7_ | TAATACGACTCACTATAGGTTATTTCGTGTA |
| HTS_11T | TTTATTTAGTTTTAGAGCTAGAAATAGCA |
| fwd_sgRNA_T7_ | TAATACGACTCACTATAGGTTATTTCGTGCA |
| HTS_11C | TTTATTTAGTTTTAGAGCTAGAAATAGCA |
| fwd_sgRNA_T7_ | TAATACGACTCACTATAGGTTATTTCGTGGT |
| HTS_12T | TTTATTTAGTTTTAGAGCTAGAAATAGCA |
| fwd_sgRNA_T7_ | TAATACGACTCACTATAGGTTATTTCGTGGC |
| HTS_12C | TTTATTTAGTTTTAGAGCTAGAAATAGCA |
| fwd_sgRNA_T7_ | TAATACGACTCACTATAGGTTATTTCGTGGG |
| HTS_12G | TTTATTTAGTTTTAGAGCTAGAAATAGCA |
| fwd_sgRNA_T7_ | TAATACGACTCACTATAGGTTATTTCGTGGA |
| HTS_13A | ATTATTTAGTTTTAGAGCTAGAAATAGCA |
| fwd_sgRNA_T7_ | TAATACGACTCACTATAGGTTATTTCGTGGA |
| HTS_13C | CTTATTTAGTTTTAGAGCTAGAAATAGCA |
| fwd_sgRNA_T7_ | TAATACGACTCACTATAGGTTATTTCGTGGA |
| HTS_13G | GTTATTTAGTTTTAGAGCTAGAAATAGCA |
| fwd_sgRNA_T7_ | TAATACGACTCACTATAGGTTCCCCCCCCGA |
| HTS_multic | TTTATTTAGTTTTAGAGCTAGAAATAGCA |
| fwd_sgRNA_T7_ | TAATACGACTCACTATAGGCGCACCCGTGGA |
| HTS_TCGCACCC_ | TTTATTTAGTTTTAGAGCTAGAAATAGCA |
| odd | |
| fwd_sgRNA_T7_ | TAATACGACTCACTATAGGCTCGCACGTGGA |
| HTS_CCTCGCAC_ | TTTATTTAGTTTTAGAGCTAGAAATAGCA |
| odd | |
| fwd_sgRNA_T7_ | TAATACGACTCACTATAGGCCCTCGCGTGGA |
| HTS_ACCCTCGC_ | TTTATTTAGTTTTAGAGCTAGAAATAGCA |
| odd | |
| fwd_sgRNA_T7_ | TAATACGACTCACTATAGGCACCCTCGTGGA |
| HTS_GCACCCTC_ | TTTATTTAGTTTTAGAGCTAGAAATAGCA |
| odd | |
| fwd_sgRNA_T7_ | TAATACGACTCACTATAGGTCGCACCCGTGG |
| HTS_TCGCACCC_ | ATTTATTAGTTTTAGAGCTAGAAATAGCA |
| even | |
| fwd_sgRNA_T7_ | TAATACGACTCACTATAGGCCTCGCACGTGG |
| HTS_CCTCGCAC_ | ATTTATTAGTTTTAGAGCTAGAAATAGCA |
| even | |
| fwd_sgRNA_T7_ | TAATACGACTCACTATAGGACCCTCGCGTGG |
| HTS_ACCCTCGC_ | ATTTATTAGTTTTAGAGCTAGAAATAGCA |
| even | |
| fwd_sgRNA_T7_ | TAATACGACTCACTATAGGGCACCCTCGTGG |
| HTS_GCACCCTC_ | ATTTATTAGTTTTAGAGCTAGAAATAGCA |
| even | |
| fwd_sgRNA_T7_ | TAATACGACTCACTATAGGGAGTCCGAGCAG |
| HTS_EMX1 | AAGAAGAAGTTTTAGAGCTAGAAATAGCA |
| fwd_sgRNA_T7_ | TAATACGACTCACTATAGGGGAATCCCTTCT |
| HTS_FANCF | GCAGCACCGTTTTAGAGCTAGAAATAGCA |
| fwd_sgRNA_T7_ | TAATACGACTCACTATAGGGAACACAAAGCA |
| HTS_HEK293_ | TAGACTGCGTTTTAGAGCTAGAAATAGCA |
| site2 | |
| fwd_sgRNA_T7_ | TAATACGACTCACTATAGGGGCCCAGACTGA |
| HTS_HEK293_ | GCACGTGAGTTTTAGAGCTAGAAATAGCA |
| site3 | |
| fwd_sgRNA_T7_ | TAATACGACTCACTATAGGGGCACTGCGGCT |
| HTS_HEK293_ | GGAGGTGGGTTTTAGAGCTAGAAATAGCA |
| site4 | |
| fwd_sgRNA_T7_ | TAATACGACTCACTATAGGGTCATCTTAGTC |
| HTS_RNF2 | ATTACCTGGTTTTAGAGCTAGAAATAGCA |

Sequences of in vitro-edited dsDNA for high-throughput sequencing (HTS). Shown are the sequences of edited strands. Reverse complements of all sequences shown were also obtained. dsDNA substrates were obtained by annealing complementary strands as described in Materials and Methods. Oligonucleotides representing the EMX1, FANCF, HEK293 site 2, HEK293 site 3, HEK293 site 4, and RNF2 loci were originally designed for use in the gel-based deaminase assay and therefore have the same 25-nt sequence on their 5′-ends (matching that of the Cy3-primer). SEQ ID NOs: 443-494 appear from top to bottom below, respectively.

| | |
|---|---|
| Base | ACGTAAACGGCCACAAGTTCTTATTTCGTGGATT |
| sequence | TATTTATGGCATCTTCTTCAAGGACG |
| 1A | ACGTAAACGGCCACAAGTTCATATTTCGTGGATT |
| | TATTTATGGCATCTTCTTCAAGGACG |
| 1C | ACGTAAACGGCCACAAGTTCCTATTTCGTGGATT |
| | TATTTATGGCATCTTCTTCAAGGACG |
| 1G | ACGTAAACGGCCACAAGTTCGTATTTCGTGGATT |
| | TATTTATGGCATCTTCTTCAAGGACG |
| 2A | ACGTAAACGGCCACAAGTTCTAATTTCGTGGATT |
| | TATTTATGGCATCTTCTTCAAGGACG |
| 2C | ACGTAAACGGCCACAAGTTCTCATTTCGTGGATT |
| | TATTTATGGCATCTTCTTCAAGGACG |
| 2G | ACGTAAACGGCCACAAGTTCTGATTTCGTGGATT |
| | TATTTATGGCATCTTCTTCAAGGACG |
| 3T | ACGTAAACGGCCACAAGTTCTTTTTTCGTGGATT |
| | TATTTATGGCATCTTCTTCAAGGACG |
| 3C | ACGTAAACGGCCACAAGTTCTTCTTTCGTGGATT |
| | TATTTATGGCATCTTCTTCAAGGACG |
| 3G | ACGTAAACGGCCACAAGTTCTTGTTTCGTGGATT |
| | TATTTATGGCATCTTCTTCAAGGACG |
| 4A | ACGTAAACGGCCACAAGTTCTTAATTCGTGGATT |
| | TATTTATGGCATCTTCTTCAAGGACG |
| 4C | ACGTAAACGGCCACAAGTTCTTACTTCGTGGATT |
| | TATTTATGGCATCTTCTTCAAGGACG |
| 4G | ACGTAAACGGCCACAAGTTCTTAGTTCGTGGATT |
| | TATTTATGGCATCTTCTTCAAGGACG |
| 5A | ACGTAAACGGCCACAAGTTCTTATATCGTGGATT |
| | TATTTATGGCATCTTCTTCAAGGACG |
| 5C | ACGTAAACGGCCACAAGTTCTTATCTCGTGGATT |
| | TATTTATGGCATCTTCTTCAAGGACG |
| 5G | ACGTAAACGGCCACAAGTTCTTATGTCGTGGATT |
| | TATTTATGGCATCTTCTTCAAGGACG |
| 6A | ACGTAAACGGCCACAAGTTCTTATTACGTGGATT |
| | TATTTATGGCATCTTCTTCAAGGACG |
| 6C | ACGTAAACGGCCACAAGTTCTTATTCCGTGGATT |
| | TATTTATGGCATCTTCTTCAAGGACG |
| 6G | ACGTAAACGGCCACAAGTTCTTATTGCGTGGATT |
| | TATTTATGGCATCTTCTTCAAGGACG |
| 8A | ACGTAAACGGCCACAAGTTCTTATTTCATGGATT |
| | TATTTATGGCATCTTCTTCAAGGACG |
| 8T | ACGTAAACGGCCACAAGTTCTTATTTCTTGGATT |
| | TATTTATGGCATCTTCTTCAAGGACG |
| 8C | ACGTAAACGGCCACAAGTTCTTATTTCCTGGATT |
| | TATTTATGGCATCTTCTTCAAGGACG |
| 9A | ACGTAAACGGCCACAAGTTCTTATTTCGAGGATT |
| | TATTTATGGCATCTTCTTCAAGGACG |
| 9C | ACGTAAACGGCCACAAGTTCTTATTTCGCGGATT |
| | TATTTATGGCATCTTCTTCAAGGACG |
| 9G | ACGTAAACGGCCACAAGTTCTTATTTCGGGGATT |
| | TATTTATGGCATCTTCTTCAAGGACG |
| 10A | ACGTAAACGGCCACAAGTTCTTATTTCGTAGATT |
| | TATTTATGGCATCTTCTTCAAGGACG |
| 10T | ACGTAAACGGCCACAAGTTCTTATTTCGTTGATT |
| | TATTTATGGCATCTTCTTCAAGGACG |
| 10C | ACGTAAACGGCCACAAGTTCTTATTTCGTCGATT |
| | TATTTATGGCATCTTCTTCAAGGACG |
| 11A | ACGTAAACGGCCACAAGTTCTTATTTCGTGAATT |
| | TATTTATGGCATCTTCTTCAAGGACG |
| 11T | ACGTAAACGGCCACAAGTTCTTATTTCGTGTATT |
| | TATTTATGGCATCTTCTTCAAGGACG |
| 11C | ACGTAAACGGCCACAAGTTCTTATTTCGTGCATT |
| | TATTTATGGCATCTTCTTCAAGGACG |
| 12T | ACGTAAACGGCCACAAGTTCTTATTTCGTGGTTT |
| | TATTTATGGCATCTTCTTCAAGGACG |
| 12C | ACGTAAACGGCCACAAGTTCTTATTTCGTGGCTT |
| | TATTTATGGCATCTTCTTCAAGGACG |
| 12G | ACGTAAACGGCCACAAGTTCTTATTTCGTGGGTT |
| | TATTTATGGCATCTTCTTCAAGGACG |
| 13A | ACGTAAACGGCCACAAGTTCTTATTTCGTGGAAT |
| | TATTTATGGCATCTTCTTCAAGGACG |
| 13C | ACGTAAACGGCCACAAGTTCTTATTTCGTGGACT |
| | TATTTATGGCATCTTCTTCAAGGACG |
| 13G | ACGTAAACGGCCACAAGTTCTTATTTCGTGGAGT |
| | TATTTATGGCATCTTCTTCAAGGACG |
| multic | ACGTAAACGGCCACAAGTTCTTCCCCCCCCGATT |
| | TATTTATGGCATCTTCTTCAAGGACG |
| TCGCACCC_ | ACGTAAACGGCCACAAGTTTCGCACCCGTGGATT |
| odd | TATTTATGGCATCTTCTTCAAGGACG |
| CCTCGCAC_ | ACGTAAACGGCCACAAGTTCCTCGCACGTGGATT |
| odd | TATTTATGGCATCTTCTTCAAGGACG |
| ACCCTCGC_ | ACGTAAACGGCCACAAGTTACCCTCGCGTGGATT |
| odd | TATTTATGGCATCTTCTTCAAGGACG |
| GCACCCTC_ | ACGTAAACGGCCACAAGTTGCACCCTCGTGGATT |
| odd | TATTTATGGCATCTTCTTCAAGGACG |
| TCGCACCC_ | ACGTAAACGGCCACAAGTATTCGCACCCGTGGAT |
| even | TTATTATGGCATCTTCTTCAAGGACG |
| CCTCGCAC_ | ACGTAAACGGCCACAAGTATCCTCGCACGTGGAT |
| even | TTATTATGGCATCTTCTTCAAGGACG |
| ACCCTCGC_ | ACGTAAACGGCCACAAGTATACCCTCGCGTGGAT |
| even | TTATTATGGCATCTTCTTCAAGGACG |
| GCACCCTC_ | ACGTAAACGGCCACAAGTATGCACCCTCGTGGAT |
| even | TTATTATGGCATCTTCTTCAAGGACG |
| EMX1_ | GTAGGTAGTTAGGATGAATGGAAGGTTGGTAGGC |
| invitro | CTGAGTCCGAGCAGAAGAAGAAGGGCTCCCATCA |
| | CATCAACCGGTG |
| FANCF_ | GTAGGTAGTTAGGATGAATGGAAGGTTGGTACTC |
| invitro | ATGGAATCCCTTCTGCAGCACCTGGATCGCTTTT |
| | CCGAGCTTCTGG |
| HEK293_ | GTAGGTAGTTAGGATGAATGGAAGGTTGGTAAAC |
| site2 | TGGAACACAAAGCATAGACTGCGGGGGGGGCCAG |
| invitro | CCTGAATAGCTG |
| HEK293_ | GTAGGTAGTTAGGATGAATGGAAGGTTGGTACTT |
| site3 | GGGGCCCAGACTGAGCACGTGATGGCAGAGGAAA |
| invitro | GGAAGCCCTGCT |
| HEK293_ | GTAGGTAGTTAGGATGAATGGAAGGTTGGTACCG |
| site4 | GTGGCACTGCGGCTGGAGGTGGGGGTTAAAGCG |
| invitro | GAGACTCTGGTGC |
| RNF2_ | GTAGGTAGTTAGGATGAATGGAAGGTTGGTATGG |
| invitro | CAGTCATCTTAGTCATTACCTGAGGTGTTCGTTG |
| | TAACTCATATAA |

Primers for HTS of in vitro edited dsDNA. SEQ ID NOs: 495-503 appear from top to bottom below, respectively.

| | |
|---|---|
| fwd_ | ACACTCTTTCCCTACACGACGCTCTTCCGAT |
| invitro_ | CTNNNNACGTAAACGGCCACAA |
| HTS | |
| rev_ | TGGAGTTCAGACGTGTGCTCTTCCGATCTCG |
| invitro_ | TCCTTGAAGAAGATGC |
| HTS | |
| fwd_ | ACACTCTTTCCCTACACGACGCTCTTCCGAT |
| invitro_ | CTNNNNGTAGGTAGTTAGGATGAATGGAA |
| HEK_ | |
| targets | |
| rev_EMX1_ | TGGAGTTCAGACGTGTGCTCTTCCGATCTCA |
| invitro | CCGGTTGATGTGATGG |
| rev_ | TGGAGTTCAGACGTGTGCTCTTCCGATCTCC |
| FANCF_ | AGAAGCTCGGAAAAGC |
| invitro | |
| rev_ | TGGAGTTCAGACGTGTGCTCTTCCGATCTCA |
| HEK293_ | GCTATTCAGGCTGGC |
| site2_ | |
| invitro | |
| rev_ | TGGAGTTCAGACGTGTGCTCTTCCGATCTAG |
| HEK293_ | CAGGGCTTCCTTTC |
| site3_ | |
| invitro | |
| rev_ | TGGAGTTCAGACGTGTGCTCTTCCGATCTGC |
| HEK293_ | ACCAGAGTCTCCG |
| site4_ | |
| invitro | |
| rev_RNF2_ | TGGAGTTCAGACGTGTGCTCTTCCGATCTTT |
| invitro | ATATGAGTTACAACGAACACC |

Primers for HTS of on-target and off-target sites from all mammalian cell culture experiments. SEQ ID NOs: 504-579 and 1869-1900 appear from top to bottom below, respectively.

| | |
|---|---|
| fwd_EMX1_HTS | ACACTCTTTCCCTACACGACGCTCTTCCGATCTNNNNCAGCTCAGCCTGAGTGTTGA |
| rev_EMX1_HTS | TGGAGTTCAGACGTGTGCTCTTCCGATCTCTCGTGGGTTTGTGGTTGC |
| fwd_FANCF_HTS | ACACTCTTTCCCTACACGACGCTCTTCCGATCTNNNNCATTGCAGAGAGGCGTATCA |
| rev_FANCF_HTS | TGGAGTTCAGACGTGTGCTCTTCCGATCTGGGGTCCCAGGTGCTGAC |
| fwd_HEK293_site2_HTS | ACACTCTTTCCCTACACGACGCTCTTCCGATCTNNNNCCAGCCCCATCTGTCAAACT |
| rev_HEK293_site2_HTS | TGGAGTTCAGACGTGTGCTCTTCCGATCTTGAATGGATTCCTTGGAAACAATGA |
| fwd_HEK293_site3_HTS | ACACTCTTTCCCTACACGACGCTCTTCCGATCTNNNNATGTGGGCTGCCTAGAAAGG |
| rev_HEK293_site3_HTS | TGGAGTTCAGACGTGTGCTCTTCCGATCTCCCAGCCAAACTTGTCAACC |
| fwd_HEK293_site4_HTS | ACACTCTTTCCCTACACGACGCTCTTCCGATCTNNNNGAACCCAGGTAGCCAGAGAC |
| rev_HEK293_site4_HTS | TGGAGTTCAGACGTGTGCTCTTCCGATCTTCCTTTCAACCCGAACGGAG |
| fwd_RNF2_HTS | ACACTCTTTCCCTACACGACGCTCTTCCGATCTNNNNCTCTTCTTTATTTCCAGCAATGT |
| rev_RNF2_HTS | TGGAGTTCAGACGTGTGCTCTTCCGATCTGTTTTCATGTTCTAAAAATGTATCCCA |
| fwd_p53_Y163C_HTS | ACACTCTTTCCCTACACGACGCTCTTCCGATCTNNNNTACAGTACTCCCCTGCCCTC |
| rev_p53_Y163C_HTS | TGGAGTTCAGACGTGTGCTCTTCCGATCTGCTGCTCACCATCGCTATCT |
| fwd_p53_N239D_HTS | ACACTCTTTCCCTACACGACGCTCTTCCGATCTNNNNCCTCATCTTGGGCCTGTGTT |
| rev_p53_N239D_HTS | TGGAGTTCAGACGTGTGCTCTTCCGATCTAAATCGGTAAGAGGTGGGCC |
| fwd_APOE4_C158R_HTS | ACACTCTTTCCCTACACGACGCTCTTCCGATCTNNNNGCGGACATGGAGGACGTG |
| rev_APOE4_C158R_HTS | TGGAGTTCAGACGTGTGCTCTTCCGATCTCTGTTCCACCAGGGGCCC |
| fwd_EMX1_off1_HTS | ACACTCTTTCCCTACACGACGCTCTTCCGATCTNNNNTGCCCAATCATTGATGCTTTT |
| rev_EMX1_off1_HTS | TGGAGTTCAGACGTGTGCTCTTCCGATCTAGAAACATTTACCATAGACTATCACCT |
| fwd_EMX1_off2_HTS | ACACTCTTTCCCTACACGACGCTCTTCCGATCTNNNNAGTAGCCTCTTTCTCAATGTGC |
| rev_EMX1_off2_HTS | TGGAGTTCAGACGTGTGCTCTTCCGATCTGCTTTCACAAGGATGCAGTCT |
| fwd_EMX1_off3_HTS | ACACTCTTTCCCTACACGACGCTCTTCCGATCTNNNNGAGCTAGACTCCGAGGGGA |
| rev_EMX1_off3_HTS | TGGAGTTCAGACGTGTGCTCTTCCGATCTTCCTCGTCCTGCTCTCACTT |
| fwd_EMX1_off4_HTS | ACACTCTTTCCCTACACGACGCTCTTCCGATCTNNNNAGAGGCTGAAGAGGAAGACCA |
| rev_EMX1_off4_HTS | TGGAGTTCAGACGTGTGCTCTTCCGATCTGGCCCAGCTGTGCATTCTAT |
| fwd_EMX1_off6_HTS | ACACTCTTTCCCTACACGACGCTCTTCCGATCTNNNNCCAAGAGGGCCAAGTCCTG |
| rev_EMX1_off6_HTS | TGGAGTTCAGACGTGTGCTCTTCCGATCTCAGCGAGGAGTGACAGCC |
| fwd_EMX1_off7_HTS | ACACTCTTTCCCTACACGACGCTCTTCCGATCTNNNNCACTCCACCTGATCTCGGGG |
| rev_EMX1_off7_HTS | TGGAGTTCAGACGTGTGCTCTTCCGATCTCGAGGAGGGAGGGAGCAG |
| fwd_EMX1_off8_HTS | ACACTCTTTCCCTACACGACGCTCTTCCGATCTNNNNACCACAAATGCCCAAGAGAC |
| rev_EMX1_off8_HTS | TGGAGTTCAGACGTGTGCTCTTCCGATCTGACACAGTCAAGGGCCGG |
| fwd_EMX1_off9_HTS | ACACTCTTTCCCTACACGACGCTCTTCCGATCTNNNNCCCACCTTTGAGGAGGCAAA |
| rev_EMX1_off9_HTS | TGGAGTTCAGACGTGTGCTCTTCCGATCTTTCCATCTGAGAAGAGAGTGGT |
| fwd_EMX1_off10_HTS | ACACTCTTTCCCTACACGACGCTCTTCCGATCTNNNNGTCATACCTTGGCCCTTCCT |
| rev_EMX1_off10_HTS | TGGAGTTCAGACGTGTGCTCTTCCGATCTTCCCTAGGCCCACACCAG |
| fwd_FANCF_off1_HTS | ACACTCTTTCCCTACACGACGCTCTTCCGATCTNNNNAACCCACTGAAGAAGCAGGG |
| rev_FANCF_off1_HTS | TGGAGTTCAGACGTGTGCTCTTCCGATCTGGTGCTTAATCCGGCTCCAT |
| fwd_FANCF_off2_HTS | ACACTCTTTCCCTACACGACGCTCTTCCGATCTNNNNTCCAGTGTTTCCATCCCGAA |
| rev_FANCF_off2_HTS | TGGAGTTCAGACGTGTGCTCTTCCGATCTCCTCTGACCTCCACAACTCT |
| fwd_FANCF_off3_HTS | ACACTCTTTCCCTACACGACGCTCTTCCGATCTNNNNCTGGGTACAGTTCTGCGTGT |
| rev_FANCF_off3_HTS | TGGAGTTCAGACGTGTGCTCTTCCGATCTTCACTCTGAGCATCGCCAAG |
| fwd_FANCF_off4_HTS | ACACTCTTTCCCTACACGACGCTCTTCCGATCTNNNNGGTTTAGAGCCAGTGAACTAGAG |
| rev_FANCF_off4_HTS | TGGAGTTCAGACGTGTGCTCTTCCGATCTGCAAGACAAAATCCTCTTTATACTTTG |
| fwd_FANCF_off5_HTS | ACACTCTTTCCCTACACGACGCTCTTCCGATCTNNNNGGGAGGGGACGGCCTTAC |
| rev_FANCF_off5_HTS | TGGAGTTCAGACGTGTGCTCTTCCGATCTGCCTCTGGCGAACATGGC |
| fwd_FANCF_off6_HTS | ACACTCTTTCCCTACACGACGCTCTTCCGATCTNNNNTCCTGGTTAAGAGCATGGGC |
| rev_FANCF_off6_HTS | TGGAGTTCAGACGTGTGCTCTTCCGATCTGATTGAGTCCCCACAGCACA |
| fwd_FANCF_off_HTS | ACACTCTTTCCCTACACGACGCTCTTCCGATCTNNNNCCAGTGTTTCCCATCCCCAA |
| rev_FANCF_off_HTS | TGGAGTTCAGACGTGTGCTCTTCCGATCTTGACCTCCACAACTGGAAAAT |
| fwd_FANCF_off8_HTS | ACACTCTTTCCCTACACGACGCTCTTCCGATCTNNNNGCTTCCAGACCCACCTGAAG |
| rev_FANCF_off8_HTS | TGGAGTTCAGACGTGTGCTCTTCCGATCTACCGAGGAAAATTGCTTGTCG |
| fwd_HEK293_site2_off1_HTS | ACACTCTTTCCCTACACGACGCTCTTCCGATCTNNNNGTGTGGAGAGTGAGTAAGCCA |
| rev_HEK293_site2_off1_HTS | TGGAGTTCAGACGTGTGCTCTTCCGATCTACGGTAGGATGATTTCAGGCA |
| fwd_HEK293_site2_off2_HTS | ACACTCTTTCCCTACACGACGCTCTTCCGATCTNNNNCACAAAGCAGTGTAGCTCAGG |
| rev_HEK293_site2_off2_HTS | TGGAGTTCAGACGTGTGCTCTTCCGATCTTTTTTGGTACTCGAGTGTTATTCAG |
| fwd_HEK293_site3_off1_HTS | ACACTCTTTCCCTACACGACGCTCTTCCGATCTNNNNTCCCCTGTTGACCTGGAGAA |
| rev_HEK293_site3_off1_HTS | TGGAGTTCAGACGTGTGCTCTTCCGATCTCACTGTACTTGCCCTGACCA |
| fwd_HEK293_site3_off2_HTS | ACACTCTTTCCCTACACGACGCTCTTCCGATCTNNNNTTGGTGTTGACAGGGAGCAA |
| rev_HEK293_site3_off2_HTS | TGGAGTTCAGACGTGTGCTCTTCCGATCTCTGAGATGTGGGCAGAAGGG |
| fwd_HEK293_site3_off3_HTS | ACACTCTTTCCCTACACGACGCTCTTCCGATCTNNNNTGAGAGGGAACAGAAGGGCT |
| rev_HEK293_site3_off3_HTS | TGGAGTTCAGACGTGTGCTCTTCCGATCTGTCCAAAGGCCCAAGAACCT |
| fwd_HEK293_site3_off4_HTS | ACACTCTTTCCCTACACGACGCTCTTCCGATCTNNNNTCCTAGCACTTTGGAAGGTCG |
| rev_HEK293_site3_off4_HTS | TGGAGTTCAGACGTGTGCTCTTCCGATCTGCTCATCTTAATCTGCTCAGCC |
| fwd_HEK293_site3_off5_HTS | ACACTCTTTCCCTACACGACGCTCTTCCGATCTNNNNAAAGGAGCAGCTCTTCCTGG |
| rev_HEK293_site3_off5_HTS | TGGAGTTCAGACGTGTGCTCTTCCGATCTGTCTGCACCATCTCCCACAA |
| fwd_HEK293_site4_off1_HTS | ACACTCTTTCCCTACACGACGCTCTTCCGATCTNNNNGGCATGGCTTCTGAGACTCA |
| rev_HEK293_site4_off1_HTS | TGGAGTTCAGACGTGTGCTCTTCCGATCTGTCTCCCTTGCACTCCCTGTCTTT |
| fwd_HEK293_site4_off2_HTS | ACACTCTTTCCCTACACGACGCTCTTCCGATCTNNNNTTTGGCAATGGAGGCATTGG |
| rev_HEK293_site4_off2_HTS | TGGAGTTCAGACGTGTGCTCTTCCGATCTGAAGAGGCTGCCCATGAGAG |
| fwd_HEK293_site4_off3_HTS | ACACTCTTTCCCTACACGACGCTCTTCCGATCTNNNNGGTCTGAGGCTCGAATCCTG |
| rev_HEK293_site4_off3_HTS | TGGAGTTCAGACGTGTGCTCTTCCGATCTCTGTGGCCTCCATATCCCTG |
| fwd_HEK293_site4_off4_HTS | ACACTCTTTCCCTACACGACGCTCTTCCGATCTNNNNTTTCCACCAGAACTCAGCCC |
| rev_HEK293_site4_off4_HTS | TGGAGTTCAGACGTGTGCTCTTCCGATCTCCTCGGTTCCTCCACAACAC |
| fwd_HEK293_site4_off5_HTS | ACACTCTTTCCCTACACGACGCTCTTCCGATCTNNNNCACGGGAAGGACAGGAGAA¢ |
| rev_HEK293_site4_off5_HTS | TGGAGTTCAGACGTGTGCTCTTCCGATCTGCAGGGGAGGGATAAAGCAG |
| fwd_HEK293_site4_off6_HTS | ACACTCTTTCCCTACACGACGCTCTTCCGATCTNNNNCCACGGGAGATGGCTTATGT |
| rev_HEK293_site4_off6_HTS | TGGAGTTCAGACGTGTGCTCTTCCGATCTCACATCCTCACTGTGCCACT |
| fwd_HEK293_site4_off_HTS | ACACTCTTTCCCTACACGACGCTCTTCCGATCTNNNNGTCAGTCTCGGCCCCTCA |
| rev_HEK293_site4_off_HTS | TGGAGTTCAGACGTGTGCTCTTCCGATCTGCCACTGTAAAGCTCTTGGG |
| fwd_HEK293_site4_off8_HTS | ACACTCTTTCCCTACACGACGCTCTTCCGATCTNNNNAGGGTAGAGGGACAGAGCTG |
| rev_HEK293_site4_off8_HTS | TGGAGTTCAGACGTGTGCTCTTCCGATCTGGACCCCACATAGTCAGTGC |
| fwd_HEK293_site4_off9_HTS | ACACTCTTTCCCTACACGACGCTCTTCCGATCTNNNNGCTGTCAGCCCTATCTCCATC |
| rev_HEK293_site4_off9_HTS | TGGAGTTCAGACGTGTGCTCTTCCGATCTTGGGCAATTAGGACAGGGAC |
| fwd_HEK293_site4_off10_HTS | ACACTCTTTCCCTACACGACGCTCTTCCGATCTNNNNGCAGCGGAGGAGGTAGATTG |
| rev_HEK293_site4_off10_HTS | TGGAGTTCAGACGTGTGCTCTTCCGATCTCTCAGTACCTGGAGTCCCGA |
| fwd_HEK2_ChIP_off1_HTS | ACACTCTTTCCCTACACGACGCTCTTCCGATCTNNNNGACAGGCTCAGGAAAGCTGT |
| rev_HEK2_ChIP_off1_HTS | TGGAGTTCAGACGTGTGCTCTTCCGATCTACACAAGCCTTTCTCCAGGG |
| fwd_HEK2_ChIP_off2_HTS | ACACTCTTTCCCTACACGACGCTCTTCCGATCTNNNNAATAGGGGGTGAGACTGGGG |
| rev_HEK2_ChIP_off2_HTS | TGGAGTTCAGACGTGTGCTCTTCCGATCTGCCTCAGACGAGACTTGAGG |
| fwd_HEK2_ChIP_off3_HTS | ACACTCTTTCCCTACACGACGCTCTTCCGATCTNNNNGGCCAGCAGGAAAGGAATCT |
| rev HEK2 ChIP off3 HTS | TGGAGTTCAGACGTGTGCTCTTCCGATCTTGACTGCACCTGTAGCCATG |
| fwd_HEK2_ChIP_off4_HTS | ACACTCTTTCCCTACACGACGCTCTTCCGATCTNNNNTCAAGGAAATCACCCTGCCC |
| rev_HEK2_ChIP_off4_HTS | TGGAGTTCAGACGTGTGCTCTTCCGATCTAACTTCCTTGGTGTGCAGCT |
| fwd_HEK2_ChIP_off5_HTS | ACACTCTTTCCCTACACGACGCTCTTCCGATCTNNNNATGGGCTCAGCTACGTCATG |
| rev HEK2 ChIP off5 HTS | TGGAGTTCAGACGTGTGCTCTTCCGATCTAATAGCAGTGTGGTGGGCAA |
| fwd_HEK3_ChIP_off1_HTS | ACACTCTTTCCCTACACGACGCTCTTCCGATCTNNNNCGCACATCCCTTGTCTCTCT |
| rev_HEK3_ChIP_off1_HTS | TGGAGTTCAGACGTGTGCTCTTCCGATCTCTACTGGAGCACACCCCAAG |
| fwd_HEK3_ChIP_off2_HTS | ACACTCTTTCCCTACACGACGCTCTTCCGATCTNNNNTGGGTCACGTAGCTTTGGTC |
| rev_HEK3_ChIP_off2_HTS | TGGAGTTCAGACGTGTGCTCTTCCGATCTTGGTGGCCATGTGCAACTAA |
| fwd_HEK3_ChIP_off3_HTS | ACACTCTTTCCCTACACGACGCTCTTCCGATCTNNNNCTACTACGTGCCAGCTCAGG |
| rev_HEK3_ChIP_off3_HTS | TGGAGTTCAGACGTGTGCTCTTCCGATCTACCTCCCCTCCTCACTAACC |
| fwd_HEK3_ChIP_off4_HTS | ACACTCTTTCCCTACACGACGCTCTTCCGATCTNNNNGCCTCAGCTCCATTTCCTGT |
| rev_HEK3_ChIP_off4_HTS | TGGAGTTCAGACGTGTGCTCTTCCGATCTAACCTTTATGGCACCAGGGG |
| fwd_HEK3_ChIP_off5_HTS | ACACTCTTTCCCTACACGACGCTCTTCCGATCTNNNNGAGCTCAGCATTAGCAGGCT |
| rev_HEK3_ChIP_off5_HTS | TGGAGTTCAGACGTGTGCTCTTCCGATCTTTCCTGGCTTTCCGATTCCC |
| fwd_HEK4_ChIP_off1_HTS | ACACTCTTTCCCTACACGACGCTCTTCCGATCTNNNNGTGCAATTGGAGGAGGAGCT |
| rev_HEK4_ChIP_off1_HTS | TGGAGTTCAGACGTGTGCTCTTCCGATCTCACCAGCTACAGGCAGAACA |
| fwd_HEK4_ChIP_off3_HTS | ACACTCTTTCCCTACACGACGCTCTTCCGATCTNNNNCCTACCCCCAACACAGATGG |
| rev_HEK4_ChIP_off3_HTS | TGGAGTTCAGACGTGTGCTCTTCCGATCTCCACACAACTCAGGTCCTCC |

Sequences of single-stranded oligonucleotide donor templates (ssODNs) used in HDR studies.

EMX1 sense
(SEQ ID NO: 580)
TCATCTGTGCCCCTCCCTCCCTGGCCCAGGTGAAGGTGTGGTTCCAG
AACCGGAGGACAAAGTACAAACGGCAGAAGCTGGAGGAGGAAGGGCC
TGAGTTTGAGCAGAAGAAGAAGGGCTCCCATCACATCAACCGGTGGC
GCATTGCCACGAAGCAGGCCAATGGGGAGGACATCGATGTCACCTCC
AATGACTAGGGT
EMX1 antisense
(SEQ ID NO: 581)
ACCCTAGTCATTGGAGGTGACATCGATGTCCTCCCCATTGGCCTGCT
TCGTGGCAATGCGCCACCGGTTGATGTGATGGGAGCCCTTCTTCTTC
TGCTCAAACTCAGGCCCTTCCTCCTCCAGCTTCTGCCGTTTGTACTT
TGTCCTCCGGTTCTGGAACCACACCTTCACCTGGGCCAGGGAGGGAG
GGGCACAGATGA
HEK293 site 3 sense
(SEQ ID NO: 582)
CATGCAATTAGTCTATTTCTGCTGCAAGTAAGCATGCATTTGTAGGC
TTGATGCTTTTTTTCTGCTTCTCCAGCCCTGGCCTGGGTCAATCCTT
GGGGCTTAGACTGAGCACGTGATGGCAGAGGAAAGGAAGCCCTGCTT
CCTCCAGAGGGCGTCGCAGGACAGCTTTTCCTAGACAGGGGCTAGTA
TGTGCAGCTCCT
HEK293 site 3 antisense
(SEQ ID NO: 583)
AGGAGCTGCACATACTAGCCCCTGTCTAGGAAAAGCTGTCCTGCGAC
GCCCTCTGGAGGAAGCAGGGCTTCCTTTCCTCTGCCATCACGTGCTC
AGTCTAAGCCCCAAGGATTGACCCAGGCCAGGGCTGGAGAAGCAGAA
AAAAAGCATCAAGCCTACAAATGCATGCTTACTTGCAGCAGAAATAG
ACTAATTGCATG
HEK site 4 sense
(SEQ ID NO: 584)
GGCTGACAAAGGCCGGGCTGGGTGGAAGGAAGGGAGGAAGGGCGAGG
CAGAGGGTCCAAAGCAGGATGACAGGCAGGGGCACCGCGGCGCCCCG
GTGGCATTGCGGCTGGAGGTGGGGGTTAAAGCGGAGACTCTGGTGCT
GTGTGACTACAGTGGGGGCCCTGCCCTCTCTGAGCCCCCGCCTCCAG
GCCTGTGTGTGT
HEK site 4 antisense
(SEQ ID NO: 585)
ACACACACAGGCCTGGAGGGGGGGGCTCAGAGAGGGCAGGGCCCCCA
CTGTAGTCACACAGCACCAGAGTCTCCGCTTTAACCCCCACCTCCAG
CCGCAATGCCACCGGGGCGCCGCGGTGCCCCTGCCTGTCATCCTGCT
TTGGACCCTCTGCCTCGCCCTTCCTCCCTTCCTTCCACCCAGCCCGG
CCTTTGTCAGCC
APOE4 sense
(SEQ ID NO: 743)
AGCACCGAGGAGCTGCGGGTGCGCCTCGCCTCCCACCTGCGCAAGCT
GCGTAAGCGGCTCCTCCGCGATGCCGATGACCTGCAGAAGTGCCTGG
CAGTGTACCAGGCCGGGGCCCGCGAGGGCGCCGAGCGCGGCCTCAGC
GCCATCCGCGAGCGCCTGGGGCCCCTGGTGGAACAGGGCCGCGTGCG
GGCCGCCACTGT
APOE4 antisense
(SEQ ID NO: 744)
ACAGTGGCGGCCCGCACGCGGCCCTGTTCCACCAGGGGCCCCAGGCG
CTCGCGGATGGCGCTGAGGCCGCGCTCGGCGCCCTCGCGGGCCCCGG
CCTGGTACACTGCCAGGCACTTCTGCAGGTCATCGGCATCGCGGAGG
AGCCGCTTACGCAGCTTGCGCAGGTGGGAGGCGAGGCGCACCCGCAG
CTCCTCGGTGCT
p53 Y163C sense
(SEQ ID NO: 745)
ACTCCCCTGCCCTCAACAAGATGTTTTGCCAACTGGCCAAGACCTGC
CCTGTGCAGCTGTGGGTTGATTCCACACCCCCGCCCGGCACCCGCGT
CCGCGCCATGGCCATCTACAAGCAGTCACAGCACATGACGGAGGTTG
TGAGGCGCTGCCCCCACCATGAGCGCTGCTCAGATAGCGATGGTGAG
CAGCTGGGGCTG
p53 Y163C antisense
(SEQ ID NO: 746)
CAGCCCCAGCTGCTCACCATCGCTATCTGAGCAGCGCTCATGGTGGG
GGCAGCGCCTCACAACCTCCGTCATGTGCTGTGACTGCTTGTAGATG
GCCATGGCGCGGACGCGGGTGCCGGGGGGGGGTGTGGAATCAACCCA
CAGCTGCACAGGGCAGGTCTTGGCCAGTTGGCAAAACATCTTGTTGA
GGGCAGGGGAGT

Deaminase Gene gBlocks Gene Fragments

hAID
(SEQ ID NO: 586)
CATCCTTGGTACCGAGCTCGGATCCAGCCACCATGGATAGCCTCTTG
ATGAATAGACGCAAGTTCCTGTATCAGTTTAAAAACGTGAGATGGGC
AAAAGGCCGACGAGAGACATATCTGTGCTATGTCGTTAAGCGCAGAG
ATTCAGCCACCAGTTTCTCTCTCGACTTCGGCTACCTGCGGAACAAG
AATGGTTGCCATGTTGAGCTCCTGTTCCTGAGGTATATCAGCGACTG
GGATTTGGACCCAGGGCGGTGCTATAGGGTGACATGGTTTACCTCCT
GGTCACCTTGTTATGACTGCGCGCGGCATGTTGCCGATTTTCTGAGA
GGGAACCCTAACCTGTCTCTGAGGATCTTCACCGCGCGACTGTACTT
CTGTGAGGACCGGAAAGCCGAACCCGAGGGACTGAGACGCCTCCACA
GAGCGGGTGTGCAGATTGCCATAATGACCTTTAAGGACTACTTCTAC
TGCTGGAACACCTTCGTCGAAAATCACGAGCGGACTTTCAAGGCTTG
GGAAGGATTGCATGAAAACAGCGTCAGGCTTTCCAGGCAGCTTCGCC
GCATTCTTCTCCCGTTGTACGAGGTTGATGACCTCAGAGATGCCTTT
AGAACACTGGGACTGTAGGCGGCCGCTCGATTGGTTTGGTGTGGCTC
TAA
rAPOBEC1 (mammalian)
(SEQ ID NO: 587)
CATCCTTGGTACCGAGCTCGGATCCAGCCACCATGAGCTCAGAGACT
GGCCCAGTGGCTGTGGACCCCACATTGAGACGGCGGATCGAGCCCCA
TGAGTTTGAGGTATTCTTCGATCCGAGAGAGCTCCGCAAGGAGACCT
GCCTGCTTTACGAAATTAATTGGGGGGGCCGGCACTCCATTTGGCGA
CATACATCACAGAACACTAACAAGCACGTCGAAGTCAACTTCATCGA
GAAGTTCACGACAGAAAGATATTTCTGTCCGAACACAAGGTGCAGCA
TTACCTGGTTTCTCAGCTGGAGCCCATGCGGCGAATGTAGTAGGGCC
ATCACTGAATTCCTGTCAAGGTATCCCCACGTCACTCTGTTTATTTA
CATCGCAAGGCTGTACCACCACGCTGACCCCCGCAATCGACAAGGCC
TGCGGGATTTGATCTCTTCAGGTGTGACTATCCAAATTATGACTGAG
CAGGAGTCAGGATACTGCTGGAGAAACTTTGTGAATTATAGCCCGAG
TAATGAAGCCCACTGGCCTAGGTATCCCCATCTGTGGGTACGACTGT
ACGTTCTTGAACTGTACTGCATCATACTGGGCCTGCCTCCTTGTCTC
AACATTCTGAGAAGGAAGCAGCCACAGCTGACATTCTTTACCATCGC
TCTTCAGTCTTGTCATTACCAGCGACTGCCCCCACACATTCTCTGGG
CCACCGGGTTGAAATGAGCGGCCGCTCGATTGGTTTGGTGTGGCTCT
AA
pmCDA1
(SEQ ID NO: 588)
CATCCTTGGTACCGAGCTCGGATCCAGCCACCATGACAGACGCTGAA
TATGTTAGGATCCATGAAAAACTGGATATCTATACATTTAAGAAGCA
GTTCTTCAATAACAAAAAGTCAGTATCTCACAGATGCTATGTCCTGT
TCGAACTCAAGAGAAGAGGAGAAAGGCGGGCCTGTTTCTGGGGGTAC
GCGGTTAATAAACCCCAGTCCGGGACCGAGAGGGGGATTCACGCCGA
GATCTTTTCAATTAGGAAGGTTGAAGAGTATCTTCGCGACAATCCCG
GTCAGTTCACAATTAACTGGTACAGCTCCTGGAGCCCTTGCGCTGAT
TGCGCCGAGAAAATACTGGAATGGTACAACCAGGAGTTGAGAGGCAA
TGGCGACACTCTCAAGATTTGGGCTTGCAAGCTTTACTACGAGAAGA
ACGCGAGAAATCAGATTGGCTTGTGGAACCTCAGGGACAACGGGGTC
GGGTTGAATGTTATGGTGTCCGAACATTACCAGTGCTGTAGAAAGAT
CTTCATTCAGTCCAGTCACAATCAGCTGAACGAGAACAGATGGCTGG
AGAAAACACTGAAACGGGCAGAGAAAAGGCGCTCAGAGCTGAGTATC
ATGATCCAGGTCAAAATCCTGCATACAACCAAAAGCCCGGCTGTATA
AGCGGCCGCTCGATTGGTTTGGTGTGGCTCTAA
haPOBEC3G
(SEQ ID NO: 589)
CATCCTTGGTACCGAGCTCGGATCCAGCCACCATGGAGCTGAAGTAT
CACCCTGAGATGCGGTTTTTCCACTGGTTTAGTAAGTGGCGCAAACT
TCATCGGGATCAGGAGTATGAAGTGACCTGGTATATCTCTTGGTCTC
CCTGCACAAAATGTACACGCGACATGGCCACATTTCTGGCCGAGGAT
CCAAAGGTGACGCTCACAATCTTTGTGGCCCGCCTGTATTATTTCTG
GGACCCGGATTATCAGGAGGCACTTAGGTCATTGTGCCAAAAGCGCG
ACGGACCAGGGGCGACTATGAAAATCATGAATTATGACGAATTCCAG
CATTGCTGGAGTAAGTTTGTGTACAGCCAGCGGGAGCTGTTCGAGCC
CTGGAACAATCTTCCCAAGTACTACATACTGCTTCACATTATGTTGG
GGGAGATCCTTCGGCACTCTATGGATCCTCCTACCTTTACGTTTAAC
TTTAATAATGAGCCTTGGGTTCGGGGGCGCCATGAAACCTATTTGTG
CTACGAGGTCGAGCGGATGCATAATGATACGTGGGTCCTGCTGAATC
AGAGGAGGGGGTTTCTGTGTAACCAGGCTCCACATAAACATGGATTT
CTCGAGGGGCGGCACGCCGAACTGTGTTTCCTTGATGTGATACCTTT
CTGGAAGCTCGACCTTGATCAAGATTACAGGGTGACGTGTTTCACCT
CCTGGTCACCCTGCTTCAGTTGCGCCCAAGAGATGGCTAAATTTATC
AGTAAGAACAAGCATGTGTCCCTCTGTATTTTTACAGCCAGAATTTA
TGATGACCAGGGCCGGTGCCAGGAGGGGCTGCGGACACTCGCTGAGG
GGGGCGCGAAGATCAGCATAATGACATACTCCGAATTCAAACACTGT
TGGGACACTTTTGTGGACCACCAGGGCTGCCCATTTCAGCCGTGGGA
TGGGCTCGACGAACATAGTCAGGATCTCTCAGGCCGGCTGCGAGCCA
TATTGCAGAACCAGGAGAATTAGGCGGCCGCTCGATTGGTTTGGTGT
GGCTCTAA
rAPOBEC1(E. Coli)
(SEQ ID NO: 590)
GGCCGGGGATTCTAGAAATAATTTTGTTTAACTTTAAGAAGGAGATA
TACCATGGATGTCTTCTGAAACCGGTCCGGTTGCGGTTGACCCGACC
CTGCGTCGTCGTATCGAACCGCACGAATTCGAAGTTTTCTTCGACCC
GCGTGAACTGCGTAAAGAAACCTGCCTGCTGTACGAAATCAACTGGG
GTGGTCGTCACTCTATCTGGGGTCACACCTCTCAGAAGACCAACAAA
CACGTTGAAGTTAACTTCATCGAAAAATTCACCACCGAACGTTACTT
CTGCCCGAACACCCGTTGCTCTATCACCTGGTTCCTGTCTTGGTCTC
CGTGCGGTGAATGCTCTCGTGCGATCACCGAATTCCTGTCTCGTTAC
CCGCACGTTACCCTGTTCATCTACATCGCGCGTCTGTACCACCACGC
GGACCCGCGTAACCGTCAGGGTCTGCGTGACCTGATCTCTTCTGGTG
TTACCATCCAGATCATGACCGAACAGGAATCTGGTTACTGCTGGCGT
AACTTCGTTAACTACTCTCCGTCTAACGAAGCGCACTGGCCGCGTTA
CCCGCACCTGTGGGTTCGTCTGTACGTTCTGGAACTGTACTGCATCA
TCCTGGGTCTGCCGCCGTGCCTGAACATCCTGCGTCGTAAACAGCCG
CAGCTGACCTTCTTCACCATCGCGCTGCAGTCTTGCCACTACCAGCG
TCTGCCGCCGCACATCCTGTGGGCGACCGGTCTGAAAGGTGGTAGTG
GAGGGAGCGGCGGTTCAATGGATAAGAAATAC

### Amino Acid Sequences of NBE1, NBE2, and NBE3.

NBE1 for E. Coli expression (His₆-rAPOBEC1-XTEN-dCas9)
(SEQ ID NO: 591)
MGSSHHHHHHMSSETGPVAVDPTLRRRIEPHEFEVFFDPRELRKETCLLYEINWGGRHSIWRHTSQNTN
KHVEVNFIEKFTTERYFCPNTRCSITWFLSWSPCGECSRAITEFLSRYPHVTLFIYIARLYHHADPRNRQGL
RDLISSGVTIQIMTEQESGYCWRNFVNYSPSNEAHWPRYPHLWVRLYVLELYCIILGLPPCLNILRRKQPQ
LTFFTIALQSCHYQRLPPHILWATGLKSGSETPGTSESATPESDKKYSIGLAIGTNSVGWAVITDEYKVPSK
KFKVLGNTDRHSIKKNLIGALLFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLSDILRVNTEITKAPLSA
SMIKRYDEHHQDLTLLKALVRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDK
DFLDNEENEDILEDIVLTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENTQLQNEKLYLYYLQ
NGRDMYVDQELDINRLSDYDVDAIVPQSFLKDDSIDNKVLTRSDKNRGKSDNVPSEEVVKKMKNYWRQL
LNAKLITQRKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMIAKSEQEIG
KATAKYFFYSNIMNFFKTEITLANGEIRKRPLIETNGETGEIVWDKGRDFATVRKVLSMPQVNIVKKTEVQT
GGFSKESILPKRNSDKLIARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELALPSKYVNFLYLASHYEKL
KGSPEDNEQKQLFVEQHKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSPKKKRKV
NBE1 for Mammalian expression (rAPOBEC1-XTEN-dCas9-NLS)
(SEQ ID NO: 592)
MSSETGPVAVDPTLRRRIEPHEFEVFFDPRELRKETCLLYEINWGGRHSIWRHTSQNTNKHVEVNFIEKF
TTERYFCPNTRCSITWFLSWSPCGECSRAITEFLSRYPHVTLFIYIARLYHHADPRNRQGLRDLISSGVTIQ
IMTEQESGYCWRNFVNYSPSNEAHWPRYPHLWVRLYVLELYCIILGLPPCLNILRRKQPOLTFFTIALQSC
HYQRLPPHILWATGLKSGSETPGTSESATPESDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDLNPDNSDVDKLF
IQLVQTYNQLFEENPINASGVDAKAILSARLSKSRRLENLIAQLPGEKKNGLFGNLIALSLGLTPNFKSNFDL
AEDAKLQLSKDTYDDDLDNLLAQIGDQYADLFLAAKNLSDAILLSDILRVNTEITKAPLSASMIKRYDEHHQ
DLTLLKALVRQQLPEKYKEIFFDQSKNGYAGYIDGGASQEEFYKFIKPILEKMDGTEELLVKLNREDLLRKQ
RTFDNGSIPHOIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSGKTILDFLKSDGFA
NRNFMQLIHDDSLTFKEDIQKAQVSGQGDSLHEHIANLAGSPAIKKGILQTVKVVDELVKVMGRHKPENIVI
EMARENQTTQKGQKNSRERMKRIEEGIKELGSQILKEHPVENTQLQNEKLYLYYLQNGRDMYVDQELDI
NRLSDYDVDAIVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQTGGFSKESILPKRN
SDKLIARKKDWDPKKYGGFDSPTVAYSVLVVAKVEKGKSKKLKSVKELLGITIMERSSFEKNPIDFLEAKG
YKEVKKDLIIKLPKYSLFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHLFTLTNLGAPAAFKYFDTTI
DRKRYTSTKEVLDATLIHQSITGLYETRIDLSQLGGDSGGSPKKKRKV
Alternative NBE1 for Mammalian expression with human APOBEC1 (hAPOBEC1-
XTEN-dCas9-NLS)
(SEQ ID NO: 5737)
MTSEKGPSTGDPTLRRRIEPWEFDVFYDPRELRKEACLLYEIKWGMSRKIWRSSGKNTTN
HVEVNFIKKFTSERDFHPSMSCSITWFLSWSPCWECSQAIREFLSRHPGVTLVIYVARLFW
HMDQQNRQGLRDLVNSGVTIQIMRASEYYHCWRNFVNYPPGDEAHWPQYPPLWMMLY
ALELHCIILSLPPCLKISRRWQNHLTFFRLHLQNCHYQTIPPHILLATGLIHPSVAWRGSETP
GTSESATPESDKKYSIGLAIGTNSVGWAVITDEYKVPSKKFKVLGNTDRHSIKKNLIGALL
FDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLAA
KNLSDAILLSDILRVNTEITKAPLSASMIKRYDEHHQDLTLLKALVRQQLPEKYKEIFFDQ
SKNGYAGYIDGGASQEEFYKFIKPILEKMDGTEELLVKLNREDLLRKQRTFDNGSIPHQIH
LGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMR
KPAFLSGEQKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVMKQLKRRRY
TGWGRLSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQKGQK
NSRERMKRIEEGIKELGSQILKEHPVENTQLQNEKLYLYYLQNGRDMYVDQELDINRLSD
YDVDAIVPQSFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVK
VITLKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEIVW
DKGRDFATVRKVLSMPQVNIVKKTEVQTGGFSKESILPKRNSDKLIARKKDWDPKKYGG
FDSPTVAYSVLVVAKVEKGKSKKLKSVKELLGITIMERSSFEKNPIDFLEAKGYKEVKKD
LIIKLPKYSLFELENGRKRMLASAGELQKGNELALPSKYVNFLYLASHYEKLKGSPEDNE
QKQLFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGDSGGSPKKK
RKV
NBE2 (rAPOBEC1-XTEN-dCas9-UGI-NLS)
(SEQ ID NO: 593)
MSSETGPVAVDPTLRRRIEPHEFEVFFDPRELRKETCLLYEINWGGRHSIWRHTSQNTNKHVEVNFIEKF
TTERYFCPNTRCSITWFLSWSPCGECSRAITEFLSRYPHVTLFIYIARLYHHADPRNRQGLRDLISSGVTIQ
IMTEQESGYCWRNFVNYSPSNEAHWPRYPHLWVRLYVLELYCIILGLPPCLNILRRKOPQLTFFTIALQSC
HYQRLPPHILWATGLKSGSETPGTSESATPESDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDLNPDNSDVDKLF
IQLVQTYNQLFEENPINASGVDAKAILSARLSKSRRLENLIAQLPGEKKNGLFGNLIALSLGLTPNFKSNFDL
AEDAKLQLSKDTYDDDLDNLLAQIGDQYADLFLAAKNLSDAILLSDILRVNTEITKAPLSASMIKRYDEHHQ
DLTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKOLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSGKTILDFLKSDGFA
NRNFMQLIHDDSLTFKEDIQKAQVSGQGDSLHEHIANLAGSPAIKKGILQTVKVVDELVKVMGRHKPENIVI
EMARENQTTQKGQKNSRERMKRIEEGIKELGSQILKEHPVENTQLQNEKLYLYYLONGRDMYVDQELDI
NRLSDYDVDAIVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQTGGFSKESILPKRN
SDKLIARKKDWDPKKYGGFDSPTVAYSVLVVAKVEKGKSKKLKSVKELLGITIMERSSFEKNPIDFLEAKG
YKEVKKDLIIKLPKYSLFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHLFTLTNLGAPAAFKYFDTTI
DRKRYTSTKEVLDATLIHQSITGLYETRIDLSQLGGDSGGSTNLSDIIEKETGKQLVIQESILMLPEEVEEVIG
NKPESDILVHTAYDESTDENVMLLTSDAPEYKPWALVIQDSNGENKIKMLSGGSPKKKRKV
NBE3 (rAPOBEC1-XTEN-Cas9n-UGI-NLS)
(SEQ ID NO: 594)
MSSETGPVAVDPTLRRRIEPHEFEVFFDPRELRKETCLLYEINWGGRHSIWRHTSQNTNKHVEVNFIEKF
TTERYFCPNTRCSITWFLSWSPCGECSRAITEFLSRYPHVTLFIYIARLYHHADPRNRQGLRDLISSGVTIQ
IMTEQESGYCWRNFVNYSPSNEAHWPRYPHLWVRLYVLELYCIILGLPPCLNILRRKQPQLTFFTIALQSC
HYQRLPPHILWATGLKSGSETPGTSESATPESDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDLNPDNSDVDKLF
IQLVQTYNQLFEENPINASGVDAKAILSARLSKSRRLENLIAQLPGEKKNGLFGNLIALSLGLTPNFKSNFDL
AEDAKLQLSKDTYDDDLDNLLAQIGDQYADLFLAAKNLSDAILLSDILRVNTEITKAPLSASMIKRYDEHHQ
DLTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSGKTILDFLKSDGFA
NRNFMQLIHDDSLTFKEDIQKAQVSGQGDSLHEHIANLAGSPAIKKGILQTVKVVDELVKVMGRHKPENIVI
EMARENQTTQKGQKNSRERMKRIEEGIKELGSQILKEHPVENTQLQNEKLYLYYLQNGRDMYVDQELDI
NRLSDYDVDHIVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQTGGFSKESILPKRN
SDKLIARKKDWDPKKYGGFDSPTVAYSVLVVAKVEKGKSKKLKSVKELLGITIMERSSFEKNPIDFLEAKG
YKEVKKDLIIKLPKYSLFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHLFTLTNLGAPAAFKYFDTTI
DRKRYTSTKEVLDATLIHQSITGLYETRIDLSQLGGDSGGSTNLSDIIEKETGKQLVIQESILMLPEEVEEVIG
NKPESDILVHTAYDESTDENVMLLTSDAPEYKPWALVIQDSNGENKIKMLSGGSPKKKRKV
pmCDA1-XTEN-dCas9-UGI (bacteria)
(SEQ ID NO: 5742)
MTDAEYVRIHEKLDIYTFKKQFFNNKKSVSHRCYVLFELKRRGERRACFWGYAVNKPQS
GTERGIHAEIFSIRKVEEYLRDNPGQFTINWYSSWSPCADCAEKILEWYNQELRGNGHTL
KIWACKLYYEKNARNQIGLWNLRDNGVGLNVMVSEHYQCCRKIFIQSSHNQLNENRWL
EKTLKRAEKRRSELSIMIQVKILHTTKSPAVSGSETPGTSESATPESDKKYSIGLAIGTNSV
GWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYH
LRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLA
EDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILE
KMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLP
NEKVLPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIVLTLT
LFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENT
QLQNEKLYLYYLQNGRDMYVDQELDINRLSDYDVDAIVPQSFLKDDSIDNKVLTRSDKN
RGKSDNVPSEEVVKKMKNYWRQLLNAKLITQRKFDNLTKAERGGLSELDKAGFIKRQL
VETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEKGKSKKLKSV
KELLGITIMERSSFEKNPIDFLEAKGYKEVKKDLIIKLPKYSLFELENGRKRMLASAGELQ
KGNELALPSKYVNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTIDRKRYTSTKEV
LDATLIHQSITGLYETRIDLSQLGGDSGGSMTNLSDIIEKETGKQLVIQESILMLPEEVEEVI
GNKPESDILVHTAYDESTDENVMLLTSDAPEYKPWALVIQDSNGENKIKML
pmCDA1-XTEN-nCas9-UGI-NLS (mammalian construct)
(SEQ ID NO: 5743)
MTDAEYVRIHEKLDIYTFKKQFFNNKKSVSHRCYVLFELKRRGERRACFWGYAVNKPQS
GTERGIHAEIFSIRKVEEYLRDNPGQFTINWYSSWSPCADCAEKILEWYNQELRGNGHTL
KIWACKLYYEKNARNQIGLWNLRDNGVGLNVMVSEHYQCCRKIFIQSSHNQLNENRWL
EKTLKRAEKRRSELSIMIQVKILHTTKSPAVSGSETPGTSESATPESDKKYSIGLAIGTNSV
GWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYH
LRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLA
EDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILE
KMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLP
NEKVLPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIVLTLT
LFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRSDKN
RGKSDNVPSEEVVKKMKNYWRQLLNAKLITQRKFDNLTKAERGGLSELDKAGFIKRQL
VETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEKGKSKKLKSV
KELLGITIMERSSFEKNPIDFLEAKGYKEVKKDLIIKLPKYSLFELENGRKRMLASAGELQ
KGNELALPSKYVNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTIDRKRYTSTKEV
LDATLIHQSITGLYETRIDLSQLGGDSGGSTNLSDIIEKETGKQLVIQESILMLPEEVEEVIG
NKPESDILVHTAYDESTDENVMLLTSDAPEYKPWALVIQDSNGENKIKMLSGGSPKKKR
KV
huAPOBEC3G-XTEN-dCas9-UGI (bacteria)
(SEQ ID NO: 5744)
MDPPTFTFNFNNEPWVRGRHETYLCYEVERMHNDTWVLLNQRRGFLCNQAPHKHGFLE
GRHAELCFLDVIPFWKLDLDQDYRVTCFTSWSPCFSCAQEMAKFISKNKHVSLCIFTARIY
DDQGRCQEGLRTLAEAGAKISIMTYSEFKHCWDTFVDHQGCPFQPWDGLDEHSQDLSGR
LRAILQSGSETPGTSESATPESDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLLKALVRQQL
PEKYKEIFFDQSKNGYAGYIDGGASQEEFYKFIKPILEKMDGTEELLVKLNREDLLRKQRT
FDNGSIPHQIHLGELHAILRRQEDFYPFLKDNREKIEKILTFRIPYYVGPLARGNSRFAWMT
RKSEETITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEISGVEDR
FNASLGTYHDLLKIIKDKDFLDNEENEDILEDIVLTLTLFEDREMIEERLKTYAHLFDDKV
MKQLKRRRYTGWGRLSRKLINGIRDKQSGKTILDFLKSDGFANRNFMQLIHDDSLTFKED
IQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQ
ELDINRLSDYDVDAIVPQSFLKDDSIDNKVLTRSDKNRGKSDNVPSEEVVKKMKNYWRQ
LLNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKLE
SEFVYGDYKVYDVRKMIAKSEQEIGKATAKYFFYSNIMNFFKTEITLANGEIRKRPLIETN
GETGEIVWDKGRDFATVRKVLSMPQVNIVKKTEVQTGGFSKESILPKRNSDKLIARKKD
WDPKKYGGFDSPTVAYSVLVVAKVEKGKSKKLKSVKELLGITIMERSSFEKNPIDFLEAK
GYKEVKKDLIIKLPKYSLFELENGRKRMLASAGELQKGNELALPSKYVNFLYLASHYEKL
KGSPEDNEQKQLFVEQHKHYLDEIIEQISEFSKRVILADANLDKVLSAYNKHRDKPIREQA
ENIIHLFTLTNLGAPAAFKYFDTTIDRKRYTSTKEVLDATLIHQSITGLYETRIDLSQLGGD
SGGSMTNLSDIIEKETGKQLVIQESILMLPEEVEEVIGNKPESDILVHTAYDESTDENVMLL
TSDAPEYKPWALVIQDSNGENKIKML
huAPOBEC3G-XTEN-nCas9-UGI-NLS (mammalian construct)
(SEQ ID NO: 5745)
MDPPTFTFNFNNEPWVRGRHETYLCYEVERMHNDTWVLLNQRRGFLCNQAPHKHGFLE
GRHAELCFLDVIPFWKLDLDQDYRVTCFTSWSPCFSCAQEMAKFISKNKHVSLCIFTARIY
DDQGRCQEGLRTLAEAGAKISIMTYSEFKHCWDTFVDHQGCPFQPWDGLDEHSQDLSGR
LRAILQSGSETPGTSESATPESDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLLKALVRQQL
PEKYKEIFFDQSKNGYAGYIDGGASQEEFYKFIKPILEKMDGTEELLVKLNREDLLRKQRT
FDNGSIPHQIHLGELHAILRRQEDFYPFLKDNREKIEKILTFRIPYYVGPLARGNSRFAWMT
RKSEETITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEISGVEDR
FNASLGTYHDLLKIIKDKDFLDNEENEDILEDIVLTLTLFEDREMIEERLKTYAHLFDDKV
MKQLKRRRYTGWGRLSRKLINGIRDKQSGKTILDFLKSDGFANRNFMQLIHDDSLTFKED
IQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKNYWRQ
LLNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKLE
SEFVYGDYKVYDVRKMIAKSEQEIGKATAKYFFYSNIMNFFKTEITLANGEIRKRPLIETN
GETGEIVWDKGRDFATVRKVLSMPQVNIVKKTEVQTGGFSKESILPKRNSDKLIARKKD
WDPKKYGGFDSPTVAYSVLVVAKVEKGKSKKLKSVKELLGITIMERSSFEKNPIDFLEAK
GYKEVKKDLIIKLPKYSLFELENGRKRMLASAGELQKGNELALPSKYVNFLYLASHYEKL
KGSPEDNEQKQLFVEQHKHYLDEIIEQISEFSKRVILADANLDKVLSAYNKHRDKPIREQA
ENIIHLFTLTNLGAPAAFKYFDTTIDRKRYTSTKEVLDATLIHQSITGLYETRIDLSQLGGD
SGGSTNLSDIIEKETGKQLVIQESILMLPEEVEEVIGNKPESDILVHTAYDESTDENVMLLT
SDAPEYKPWALVIQDSNGENKIKMLSGGSPKKKRKV
huAPOBEC3G (D316R_D317R)-XTEN-nCas9-UGI-NLS (mammalian construct)
(SEQ ID NO: 5746)
MDPPTFTFNFNNEPWVRGRHETYLCYEVERMHNDTWVLLNORRGFLCNQAPHKHGFLE
GRHAELCFLDVIPFWKLDLDQDYRVTCFTSWSPCFSCAQEMAKFISKNKHVSLCIFTARIY
RRQGRCQEGLRTLAEAGAKISIMTYSEFKHCWDTFVDHQGCPFQPWDGLDEHSQDLSGR
LRAILQSGSETPGTSESATPESDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLLKALVRQQL
PEKYKEIFFDQSKNGYAGYIDGGASQEEFYKFIKPILEKMDGTEELLVKLNREDLLRKQRT
FDNGSIPHQIHLGELHAILRRQEDFYPFLKDNREKIEKILTFRIPYYVGPLARGNSRFAWMT
RKSEETITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEISGVEDR
FNASLGTYHDLLKIIKDKDFLDNEENEDILEDIVLTLTLFEDREMIEERLKTYAHLFDDKV
MKQLKRRRYTGWGRLSRKLINGIRDKQSGKTILDFLKSDGFANRNFMQLIHDDSLTFKED
IQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKNYWRQ
LLNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKLE
SEFVYGDYKVYDVRKMIAKSEQEIGKATAKYFFYSNIMNFFKTEITLANGEIRKRPLIETN
GETGEIVWDKGRDFATVRKVLSMPQVNIVKKTEVQTGGFSKESILPKRNSDKLIARKKD
WDPKKYGGFDSPTVAYSVLVVAKVEKGKSKKLKSVKELLGITIMERSSFEKNPIDFLEAK
GYKEVKKDLIIKLPKYSLFELENGRKRMLASAGELQKGNELALPSKYVNFLYLASHYEKL
KGSPEDNEQKQLFVEQHKHYLDEIIEQISEFSKRVILADANLDKVLSAYNKHRDKPIREQA
ENIIHLFTLTNLGAPAAFKYFDTTIDRKRYTSTKEVLDATLIHQSITGLYETRIDLSQLGGD
SGGSTNLSDIIEKETGKQLVIQESILMLPEEVEEVIGNKPESDILVHTAYDESTDENVMLLT
SDAPEYKPWALVIQDSNGENKIKMLSGGSPKKKRKV

### Base Calling Matlab Script

(SEQ ID NO: 595)
WTnuc =
‘GCGGACATGGAGGACGTGCGCGGCCGCCTGGTGCAGTACCGCGGCGAGG
TGCAGGCCATGCTCGGCCAGA
GCACCGAGGAGCTGCGGGTGCGCCTCGCCTCCCACCTGCGCAAGCTGCGT
AAGCGGCTCCTCCGCGATGCCGATGAC
CTGCAGAAGCGCCTGGCAGTGTACCAGGCCGGGGCCCGCGAGGGCGCCGA
GCGCGGCCTCAGCGCCATCCGCGAGCG
CCTGGGGCCCCTGGTGGAACAG’;

```
  %cycle through fastq files for different samples files=dir(‘*.fastq’);
  for d=1:20
  filename=files(d).name;
  %read fastq file
  [header,seqs,qscore]=fastqread(filename);
```

| | |
|---|---|
| seqsLength=length(seqs); | % number of sequences seqsFile= |
| strrep(filename,‘.fastq’,”); | % trims off .fastq |

%create a directory with the same name as fastq file ifexist(seqsFile,‘dir’);

error(‘Directory already exists. Please rename or move it before moving on.’);

end

| | |
|---|---|
| mkdir(seqsFile); | % make directory |
| wtLength=length(WTnuc); | % length of wildtype sequence |

```
  %% aligning back to the wildtype nucleotide sequence
  %
  % AlN is a matrix of the nucleotide alignment window=1:wtLength;
```

| | |
|---|---|
| sBLength = length(seqs); | % number of sequences |

```
  % counts number of skips nSkips = 0;
  ALN=repmat(“,[sBLength wtLength]);
  % iterate through each sequencing read for i = 1:sBLength
  %If you only have forward read fastq files leave as is
  %If you have R1 foward and R2 is reverse fastq files uncomment the
  %next four lines of code and the subsequent end statement
```

| | |
|---|---|
| % | ifmod(d,2) == 0; |
| % | reverse=seqrcomplement(seqs{i}); |
| % | [score,alignment,start]= |

swalign(reverse,WTnuc,‘Alphabet’,‘NT’);

| | |
|---|---|
| % | else |

[score,alignment,start]=swalign(seqs{i},WTnuc,‘Alphabet’,‘NT’);

| | |
|---|---|
| % | end |

```
  % length of the sequencing read len=
  length(alignment(3,:));
  % if there is a gap in the alignment , skip = 1 and we will
  % throw away the entire read skip = 0;
  for j = 1:len
if (alignment(3,j) == ‘−’ || alignment(1,j) == ‘−’) skip = 1;
```

break;

```
  end
  %in addition if the qscore for any given base in the read is
```

%below 31 the nucleotide is turned into an N (fastq qscores that are not letters)

```
  ifisletter(qscore{i}(start(1)+j−1)) else
  alignment(1,j) =‘N’;
    end
  end
  if skip == 0 && len>10
  ALN(i,  start(2):(start(2)+length(alignment)−1))=alignment(1,:);
```

end

```
  end
  % with the alignment matrices we can simply tally up the occurrences of
  % each nucleotide at each column in the alignment these
  % tallies ignore bases annotated as N
  % due to low qscores
  TallyNTD=zeros(5,wtLength); fori=1:wtLength
  TallyNTD(:,i)=[sum(ALN(:,i)==‘A’),sum(ALN(:,i)==‘C’),sum(ALN(:,i)==‘G’),sum(A
  LN(:,i)==‘T’),sum(ALN(:,i)==‘N’)];
  end
  % we then save these tally matrices in the respective folder for
  % further processing
  save(strcat(seqsFile,‘/TallyNTD’),‘TallyNTD’); dlmwrite(strcat(seqsFile,‘/TallyNTD.txt’),TallyNTD,‘precision’,
  ‘%.3f’, ‘newline’, ‘pc’); end
```

### INDEL Detection Matlab Script

(SEQ ID NO: 595)
WTnuc =
‘GCGGACATGGAGGACGTGCGCGGCCGCCTGGTGCAGTACCGCGGCGAGG
TGCAGGCCATGCTCGGCCAGA
GCACCGAGGAGCTGCGGGTGCGCCTCGCCTCCCACCTGCGCAAGCTGCGT
AAGCGGCTCCTCCGCGATGCCGATGAC
CTGCAGAAGCGCCTGGCAGTGTACCAGGCCGGGGCCCGCGAGGGCGCCGA
GCGCGGCCTCAGCGCCATCCGCGAGCG
CCTGGGGCCCCTGGTGGAACAG’;

```
  %cycle through fastq files for different samples files=dir(‘*.fastq’);
  %specify start and width of indel window as well as length of each flank indelstart=154;
  width=30; flank=10;
  for d=1:3
  filename=files(d).name;
  %read fastq file
  [header,seqs,qscore]=fastqread(filename);
```

| | |
|---|---|
| seqsLength = length(seqs); | % number of sequences seqsFile |

```
  =strcat(strrep(filename,‘.fastq’,”),‘_INDELS’);
  %create a directory with the same name as fastq file+_INDELS ifexist(seqsFile,‘dir’);
        error(‘Directory already exists. Please rename or move it before moving on.’);
```

| | |
|---|---|
| end | |
| mkdir(seqsFile); | % make directory |
| wtLength = length(WTnuc); | % length of wildtype sequence sBLength = |
| length(seqs); | % number of sequences |

```
  % initialize counters and cell arrays
  nSkips = 0; notINDEL=0;
  ins={ };
  dels={ }; NumIns=0;
  NumDels=0;
  % iterate through each sequencing read for i = 1:sBLength
      %search for 10BP sequences that should flank both sides of the “INDEL WINDOW”
    windowstart=strfind(seqs{i}, WTnuc(indelstart-flank:indelstart));
          windowend=strfind(seqs{i},WTnuc(indelstart+width:indelstart+width+flank
  ));
  % if the flanks are found proceed
  iflength(windowstart)==1&&length(windowend)==1
  %if the sequence length matches the INDEL window length save as
  %not INDEL
if windowend-windowstart==width+flank notINDEL=notINDEL+1;
  %if the sequence is two or more bases longer than the INDEL
  %window length save as an Insertion
elseif windowend-windowstart>=width+flank+2 NumIns=NumIns+1;
  ins{NumIns}=seqs{i};
  %if the sequence is two or more bases shorter than the INDEL
  %window length save as a Deletion
elseif windowend-windowstart <= width+flank−2 NumDels=NumDels+1;
  dels{NumDels}=seqs{i};
  %keep track of skipped sequences that are either one base
  %shorter or longer than the INDEL window width else
  nSkips=nSkips+1;
  end
  %keep track of skipped sequences that do not possess matching flank
  %sequences else
  nSkips=nSkips+1;
        end
  end
    fid=fopen(strcat(seqsFile,‘/summary.txt’),‘wt’);
    fprintf(fid, ‘Skipped reads %i\n not INDEL %i\n Insertions %i\n Deletions
%i\n’, [nSkips, notINDEL, NumIns, NumDels]); fclose(fid);
    save(strcat(seqsFile,‘/nSkips’),‘nSkips’); save(strcat(seqsFile,‘/notINDEL’),‘notINDEL’);
    save(strcat(seqsFile,‘/NumIns’),‘NumIns’); save(strcat(seqsFile,‘/NumDels’),‘NumDels’);
    save(strcat(seqsFile,‘/dels’),‘dels’);
    C = dels;
    fid = fopen(strcat(seqsFile, ‘/dels.txt’), ‘wt’); fprintf(fid,‘“%s”\n’,C{:});
    fclose(fid);
    save(strcat(seqsFile, ‘/ins’), ‘ins’); C = ins;
    fid = fopen(strcat(seqsFile, ‘/ins.txt’), ‘wt’); fprintf(fid,‘“%s”\n’,C{:});
    fclose(fid);
    end
```

### Example 5: Cas9 Variant Sequences

The disclosure provides Cas9 variants, for example Cas9 proteins from one or more organisms, which may comprise one or more mutations (e.g., to generate dCas9 or Cas9 nickase). In some embodiments, one or more of the amino acid residues, identified below by an asterisk, of a Cas9 protein may be mutated. In some embodiments, the D10 and/or H840 residues of the amino acid sequence provided in SEQ ID NO: 10, or a corresponding mutation in any of the amino acid sequences provided in SEQ ID NOs: 11-260, are mutated. In some embodiments, the D10 residue of the amino acid sequence provided in SEQ ID NO: 10, or a corresponding mutation in any of the amino acid sequences provided in SEQ ID NOs: 11-260, is mutated to any amino acid residue, except for D. In some embodiments, the D10 residue of the amino acid sequence provided in SEQ ID NO: 10, or a corresponding mutation in any of the amino acid sequences provided in SEQ ID NOs: 11-260, is mutated to an A. In some embodiments, the H840 residue of the amino acid sequence provided in SEQ ID NO: 10, or a corresponding residue in any of the amino acid sequences provided in SEQ ID NOs: 11-260, is an H. In some embodiments, the H840 residue of the amino acid sequence provided in SEQ ID NO: 10, or a corresponding mutation in any of the amino acid sequences provided in SEQ ID NOs: 11-260, is mutated to any amino acid residue, except for H. In some embodiments, the H840 residue of the amino acid sequence provided in SEQ ID NO: 10, or a corresponding mutation in any of the amino acid sequences provided in SEQ ID NOs: 11-260, is mutated to an A. In some embodiments, the D10 residue of the amino acid sequence provided in SEQ ID NO: 10, or a corresponding residue in any of the amino acid sequences provided in SEQ ID NOs: 11-260, is a D.

A number of Cas9 sequences from various species were aligned to determine whether corresponding homologous amino acid residues of D10 and H840 of SEQ ID NO: 10 or SEQ ID NO: 11 can be identified in other Cas9 proteins, allowing the generation of Cas9 variants with corresponding mutations of the homologous amino acid residues. The alignment was carried out using the NCBI Constraint-based Multiple Alignment Tool (COBALT (accessible at st-va.ncbi.nlm.nih.gov/tools/cobalt), with the following parameters. Alignment parameters: Gap penalties −11, −1; End-Gap penalties −5, −1. CDD Parameters: Use RPS BLAST on; Blast E-value 0.003; Find Conserved columns and Recompute on. Query Clustering Parameters: Use query clusters on; Word Size 4; Max cluster distance 0.8; Alphabet Regular.

An exemplary alignment of four Cas9 sequences is provided below. The Cas9 sequences in the alignment are: Sequence 1 (S1): SEQ ID NO: 11|WP_010922251|gi 499224711|type II CRISPR RNA-guided endonuclease Cas9 [Streptococcus pyogenes]; Sequence 2 (S2): SEQ ID NO: 12|WP_039695303|gi 746743737|type II CRISPR RNA-guided endonuclease Cas9 [Streptococcus gallolyticus]; Sequence 3 (S3): SEQ ID NO: 13|WP_045635197|gi 782887988|type II CRISPR RNA-guided endonuclease Cas9 [Streptococcus mitis]; Sequence 4 (S4): SEQ ID NO: 14|5AXW_A|gi 924443546|Staphylococcus aureus Cas9. The HNH domain (bold and underlined) and the RuvC domain (boxed) are identified for each of the four sequences. Amino acid residues 10 and 840 in S1 and the homologous amino acids in the aligned sequences are identified with an asterisk following the respective amino acid residue.

| | | | |
|---|---|---|---|
| S1 | 1 | --MDKK-YSIGLD*IGTNSVGWAVITDEYKVPSKKFKVLGNTDRHSIKKNLI--GALLFDSG--ETAEATRLKRTARRRYT | 73 |
| S2 | 1 | --MTKKNYSIGLD*IGTNSVGWAVITDDYKVPAKKMKVLGNTDKKYIKKNLL--GALLFDSG--ETAEATRLKRTARRRYT | 74 |
| S3 | 1 | --M-KKGYSIGLD*IGTNSVGFAVITDDYKVPSKKMKVLGNTDKRFIKKNLI--GALLFDEG--TTAEARRLKRTARRRYT | 73 |
| S4 | 1 | GSHMKRNYILGLD*IGITSVGYGII--DYET-----------------RDVIDAGVRLFKEANVENNEGRRSKRGARRLKR | 61 |
| S1 | 74 | RRKNRICYLQEIFSNEMAKVDDSFFHRLEESFLVEEDKKHERHPIFGNIVDEVAYHEKYPTIYHLRKKLVDSTDKADLRL | 153 |
| S2 | 75 | RRKNRLRYLQEIFANEIAKVDESFFQRLDESFLTDDDKTFDSHPIFGNKAEEDAYHQKFPTIYHLRKHLADSSEKADLRL | 154 |
| S3 | 74 | RRKNRLRYLQEIFSEEMSKVDSSFFHRLDDSFLIPEDKRESKYPIFATLTEEKEYHKQFPTIYHLRKQLADSKEKTDLRL | 153 |
| S4 | 62 | RRRHRIQRVKKLL--------------FDYNLLTD--------------------HSELSGINPYEARVKGLSQKLSEEE | 107 |
| S1 | 154 | IYLALAHMIKFRGHFLIEGDLNPDNSDVDKLFIQLVQTYNQLFEENPINASGVDAKAILSARLSKSRRLENLIAQLPGEK | 233 |
| S2 | 155 | VYLALAHMIKFRGHFLIEGELNAENTDVQKIFADFVGVYNRTFDDSHLSEITVDVASILTEKISKSRRLENLIKYYPTEK | 234 |
| S3 | 154 | IYLALAHMIKYRGHFLYEEAFDIKNNDIQKIFNEFISIYDNTFEGSSLSGQNAQVEAIFTDKISKSAKRERVLKLFPDEK | 233 |
| S4 | 108 | FSAALLHLAKRRG----------------------VHNVNEVEEDT---------------------------------- | 131 |
| S1 | 234 | KNGLFGNLIALSLGLTPNFKSNFDLAEDAKLQLSKDTYDDDLDNLLAQIGDQYADLFLAAKNLSDAILLSDILRVNTEIT | 313 |
| S2 | 235 | KNTLFGNLIALALGLQPNFKTNFKLSEDAKLQFSKDTYEEDLEELLGKIGDDYADLFTSAKNLYDAILLSGILTVDDNST | 314 |
| S3 | 234 | STGLFSEFLKLIVGNQADFKKHFDLEDKAPLQFSKDTYDEDLENLLGQIGDDFTDLFVSAKKLYDAILLSGILTVTDPST | 313 |
| S4 | 132 | -----GNELS------------------TKEQISRN-------------------------------------------- | 144 |
| S1 | 314 | KAPLSASMIKRYDEHHQDLTLLKALVRQQLPEKYKEIFFDQSKNGYAGYIDGGASQEEFYKFIKPILEKM--DGTEELLV | 391 |
| S2 | 315 | KAPLSASMIKRYVEHHEDLEKLKEFIKANKSELYHDIFKDKNKNGYAGYIENGVKQDEFYKYLKNILSKIKIDGSDYFLD | 394 |
| S3 | 314 | KAPLSASMIERYENHONDLAALKQFIKNNLPEKYDEVFSDQSKDGYAGYIDGKTTQETFYKYIKNLLSKF--EGTDYFLD | 391 |
| S4 | 145 | ----SKALEEKYVAELQ-------------------------------------------------LERLKKDG------ | 165 |
| S1 | 392 | KLNREDLLRKQRTFDNGSIPHQIHLGELHAILRRQEDFYPFLKDNREKIEKILTFRIPYYVGPLARGNSRFAWMTRKSEE | 471 |
| S2 | 395 | KIEREDFLRKQRTFDNGSIPHQIHLQEMHAILRRQGDYYPFLKEKQDRIEKILTFRIPYYVGPLVRKDSRFAWAEYRSDE | 474 |
| S3 | 392 | KIEREDFLRKQRTFDNGSIPHQIHLQEMNAILRRQGEYYPFLKDNKEKIEKILTFRIPYYVGPLARGNRDFAWLTRNSDE | 471 |
| S4 | 166 | --EVRGSINRFKTSD--------YVKEAKQLLKVQKAYHOLDQSFIDTYIDLLETRRTYYEGP--GEGSPFGW------K | 227 |
| S1 | 472 | TITPWNFEEVVDKGASAQSFIERMTNFDKNLPNEKVLPKHSLLYEYFTVYNELTKVKYVTEGMRKPAFLSGEQKKAIVDL | 551 |
| S2 | 475 | KITPWNFDKVIDKEKSAEKFITRMTLNDLYLPEEKVLPKHSHVYETYAVYNELTKIKYVNEQGKE-SFFDSNMKQEIFDH | 553 |
| S3 | 472 | AIRPWNFEEIVDKASSAEDFINKMTNYDLYLPEEKVLPKHSLLYETFAVYNELTKVKFIAEGLRDYQFLDSGQKKQIVNQ | 551 |
| S4 | 228 | DIKEW---------------YEMLMGHCTYFPEELRSVKYAYNADLYNALNDLNNLVITRDENEK---LEYYEKFQIIEN | 289 |
| $1 | 552 | LFKTNRKVTVKQLKEDYFKKIECFDSVEISGVEDR---FNASLGTYHDLLKIIKDKDFLDNEENEDILEDIVLTLTLFED | 628 |
| S2 | 554 | VFKENRKVTKEKLLNYLNKEFPEYRIKDLIGLDKENKSFNASLGTYHDLKKIL-DKAFLDDKVNEEVIEDIIKTLTLFED | 632 |
| S3 | 552 | LFKENRKVTEKDIIHYLHN-VDGYDGIELKGIEKQ---FNASLSTYHDLLKIIKDKEFMDDAKNEAILENIVHTLTIFED | 627 |
| S4 | 290 | VFKQKKKPTLKQIAKEILVNEEDIKGYRVTSTGKPEF---TNLKVYHDIKDITARKEII---ENAELLDQIAKILTIYQS | 363 |
| S1 | 629 | REMIEERLKTYAHLFDDKVMKQLKR-RRYTGWGRLSRKLINGIRDKQSGKTILDFLKSDGFANRNFMQLIHDDSLTFKED | 707 |
| S2 | 633 | KDMIHERLQKYSDIFTANQLKKLER-RHYTGWGRLSYKLINGIRNKENNKTILDYLIDDGSANRNFMQLINDDTLPFKQI | 711 |
| S3 | 628 | REMIKQRLAQYDSLFDEKVIKALTR-RHYTGWGKLSAKLINGICDKQTGNTILDYLIDDGKINRNFMQLINDDGLSFKEI | 706 |
| S4 | 364 | SEDIQEELTNLNSELTQEEIEQISNLKGYTGTHNLSLKAINLILDE------LWHTNDNQIAIFNRLKLVP--------- | 428 |
| S1 | 708 | 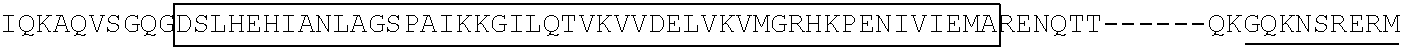 | 781 |
| S2 | 712 | 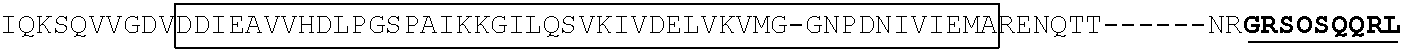 | 784 |
| S3 | 707 | 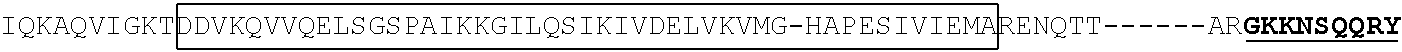 | 779 |
| S4 | 429 | 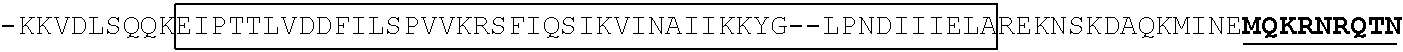 | 505 |
| S1 | 782 | KRIEEGIKELGSQIL-------KEHPVENTOLONEKLYLYYLQNGRDMYVDOELDINRLSD----YDVDH*IVPOSFLKDD | 850 |
| $2 | 785 | KKLQNSLKELGSNILNEEKPSYIEDKVENSHLQNDQLFLYYIQNGKDMYTGDELDIDHLSD----YDIDH*TIPQAFIKDD | 860 |
| S3 | 780 | KRIEDSLKILASGL---DSNILKENPTDNNOLONDRLFLYYLONGKDMYTGEALDINOLSS----YDIDH*TIPOAFIKDD | 852 |
| S4 | 506 | ERIEEIIRTTGK---------------ENAKYLIEKIKLHDMOEGKCLYSLEAIPLEDLLNNPFNYEVDH*IIPRSVSFDN | 570 |
| S1 | 851 | 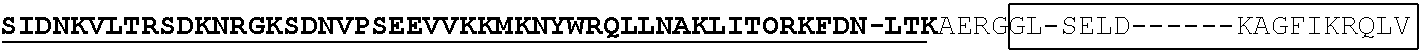 | 922 |
| S2 | 861 | 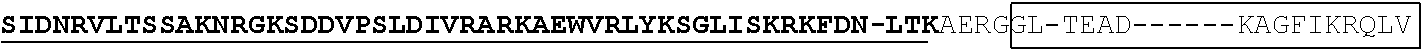 | 932 |
| S3 | 853 | 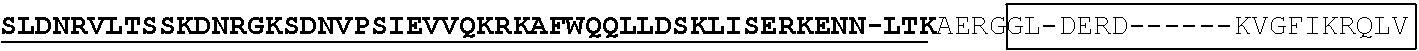 | 924 |
| $4 | 571 | 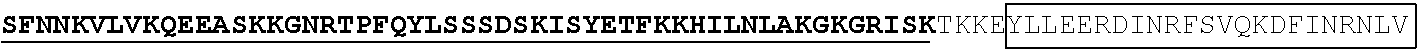 | 650 |
| S1 | 923 | 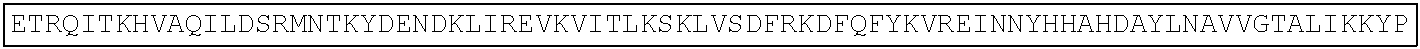 | 1002 |
| S2 | 933 | 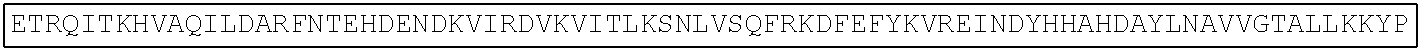 | 1012 |
| S3 | 925 | 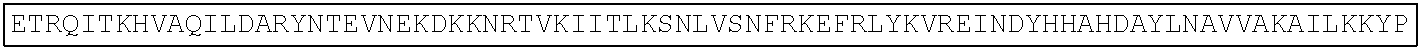 | 1004 |
| S4 | 651 | 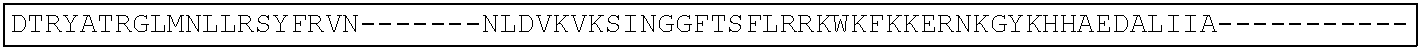 | 712 |
| S1 | 1003 | 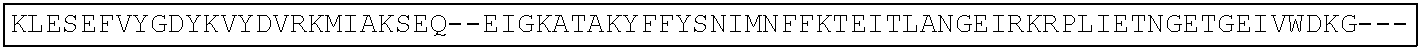 | 1077 |
| S2 | 1013 | 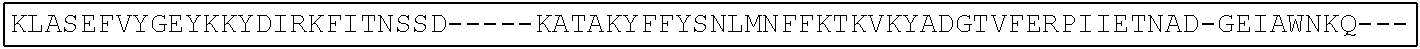 | 1083 |
| S3 | 1005 | 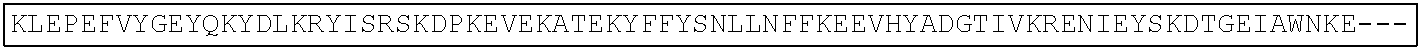 | 1081 |
| S4 | 713 | 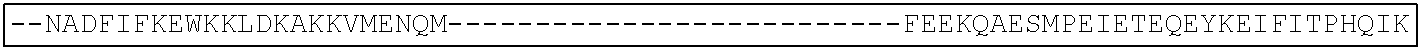 | 764 |
| S1 | 1078 | 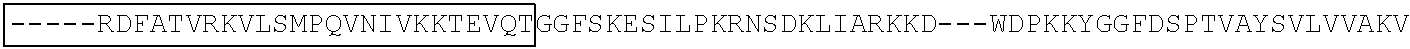 | 1149 |
| S2 | 1084 | 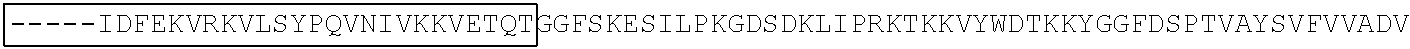 | 1158 |
| S3 | 1082 | 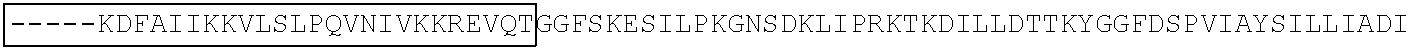 | 1156 |
| $4 | 765 | 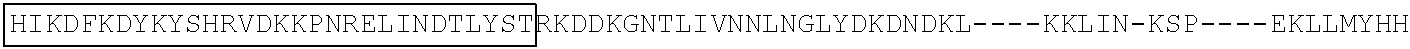 | 835 |
| S1 | 1150 | EKGKSKKLKSVKELLGITIMERSSFEKNPI-DFLEAKG-----YKEVKKDLIIKLPKYSLFELENGRKRMLASAGELQKG | 1223 |
| S2 | 1159 | EKGKAKKLKTVKELVGISIMERSFFEENPV-EFLENKG-----YHNIREDKLIKLPKYSLFEFEGGRRRLLASASELQKG | 1232 |
| S3 | 1157 | EKGKAKKLKTVKTLVGITIMEKAAFEENPI-TFLENKG-----YHNVRKENILCLPKYSLFELENGRRRLLASAKELQKG | 1230 |
| S4 | 836 | DPQTYQKLK--------LIMEQYGDEKNPLYKYYEETGNYLTKYSKKDNGPVIKKIKYYGNKLNAHLDITDDYPNSRNKV | 907 |
| S1 | 1224 | NELALPSKYVNFLYLASHYEKLKGSPEDNEQKQLFVEQHKHYLDEIIEQISEFSKRVILADANLDKVLSAYNKH------ | 1297 |
| S2 | 1233 | NEMVLPGYLVELLYHAHRADNF-----NSTEYLNYVSEHKKEFEKVLSCVEDFANLYVDVEKNLSKIRAVADSM------ | 1301 |
| S3 | 1231 | NEIVLPVYLTTLLYHSKNVHKL-----DEPGHLEYIQKHRNEFKDLLNLVSEFSQKYVLADANLEKIKSLYADN------ | 1299 |
| S4 | 908 | VKLSLKPYRFD-VYLDNGVYKFV-----TVKNLDVIK--KENYYEVNSKAYEEAKKLKKISNQAEFIASFYNNDLIKING | 979 |
| S1 | 1298 | RDKPIREQAENIIHLFTLTNLGAPAAFKYFDTTIDRKRYTSTKEVLDATLIHQSIT--------GLYETRI----DLSQL | 1365 |
| S2 | 1302 | DNFSIEEISNSFINLLTLTALGAPADFNFLGEKIPRKRYTSTKECLNATLIHQSIT--------GLYETRI----DLSKL | 1369 |
| S3 | 1300 | EQADIEILANSFINLLTFTALGAPAAFKFFGKDIDRKRYTTVSEILNATLIHQSIT--------GLYETWI----DLSKL | 1367 |
| S4 | 980 | ELYRVIGVNNDLLNRIEVNMIDITYR-EYLENMNDKRPPRIIKTIASKT---QSIKKYSTDILGNLYEVKSKKHPQIIKK | 1055 |
| S1 | 1366 | GGD | 1368 |
| S2 | 1370 | GEE | 1372 |
| S3 | 1368 | GED | 1370 |
| S4 | 1056 | G-- | 1056 |

The alignment demonstrates that amino acid sequences and amino acid residues that are homologous to a reference Cas9 amino acid sequence or amino acid residue can be identified across Cas9 sequence variants, including, but not limited to Cas9 sequences from different species, by identifying the amino acid sequence or residue that aligns with the reference sequence or the reference residue using alignment programs and algorithms known in the art. This disclosure provides Cas9 variants in which one or more of the amino acid residues identified by an asterisk in SEQ ID NOs: 11-14 (e.g., S1, S2, S3, and S4, respectively) are mutated as described herein. The residues D10 and H840 in Cas9 of SEQ ID NO: 10 that correspond to the residues identified in SEQ ID NOs: 11-14 by an asterisk are referred to herein as “homologous” or “corresponding” residues. Such homologous residues can be identified by sequence alignment, e.g., as described above, and by identifying the sequence or residue that aligns with the reference sequence or residue. Similarly, mutations in Cas9 sequences that correspond to mutations identified in SEQ ID NO: 10 herein, e.g., mutations of residues 10, and 840 in SEQ ID NO: 10, are referred to herein as “homologous” or “corresponding” mutations. For example, the mutations corresponding to the D10A mutation in SEQ ID NO: 10 or S1 (SEQ ID NO: 11) for the four aligned sequences above are D11A for S2, D10A for S3, and D13A for S4; the corresponding mutations for H840A in SEQ ID NO: 10 or S1 (SEQ ID NO: 11) are H850A for S2, H842A for S3, and H560A for S4.

A total of 250 Cas9 sequences (SEQ ID NOs: 11-260) from different species were aligned using the same algorithm and alignment parameters outlined above. Amino acid residues homologous to residues 10, and 840 of SEQ ID NO: 10 were identified in the same manner as outlined above. The alignments are provided below. The HNH domain (bold and underlined) and the RuvC domain (boxed) are identified for each of the four sequences. Single residues corresponding to amino acid residues 10, and 840 in SEQ ID NO: 10 are boxed in SEQ ID NO: 11 in the alignments, allowing for the identification of the corresponding amino acid residues in the aligned sequences.

Lengthy table referenced here
US20250270527A1-20250828-T00001
Please refer to the end of the specification for access instructions.

### Example 6: Next Generation C to T Editors

Other families of cytidine deaminases as alternatives to base etitor 3 (BE3) constructs were examined. The different C to T editors were developed to have a narrow or different editing window, alternate sequence specificity to expand targetable substrates, and to have higher activity.

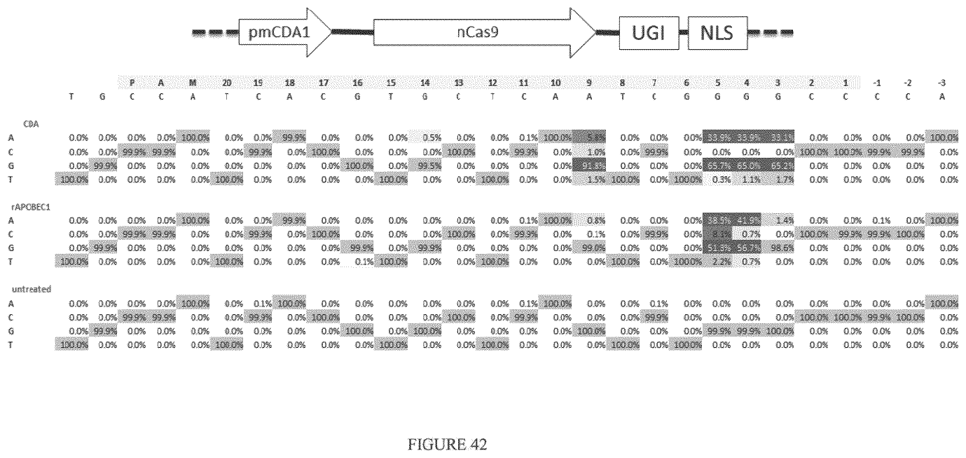

Using the methods described in Example 4, the pmCDA1 (cytidine deaminase 1 from Petromyzon marinus) activity at the HeK-3 site is evaluated (FIG. 42). The pmCDA1-nCas9-UGI-NLS (nCas9 indicates the Cas9 nickase described herein) construct is active on some sites (e.g., the C bases on the complementary strand at position 9, 5, 4, and 3) that are not accessible with rAPOBEC1 (BE3).

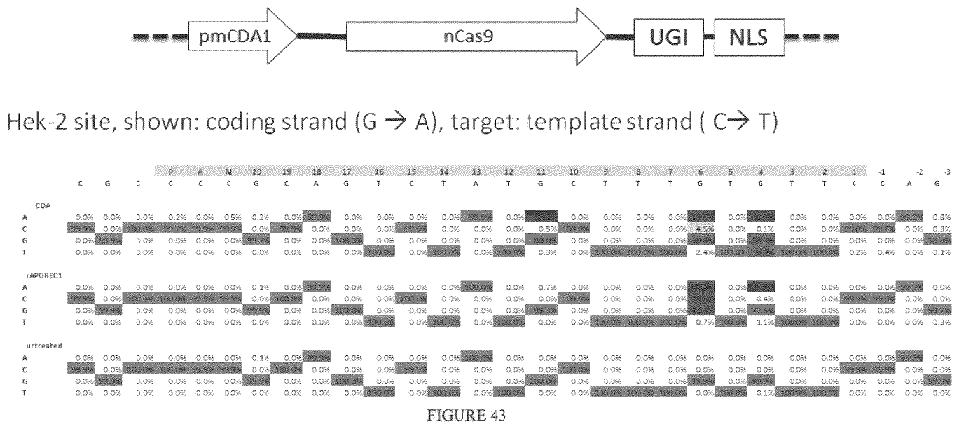

The pmCDA1 activity at the HeK-2 site is given in FIG. 43. The pmCDA1-XTEN-nCas9-UGI-NLS construct is active on sites adjacent to “G,” while rAPOBEC1 analog (BE3 construct) has low activity on “C”'s that are adjacent to “G”s, e.g., the C base at position 11 on the complementary strand.

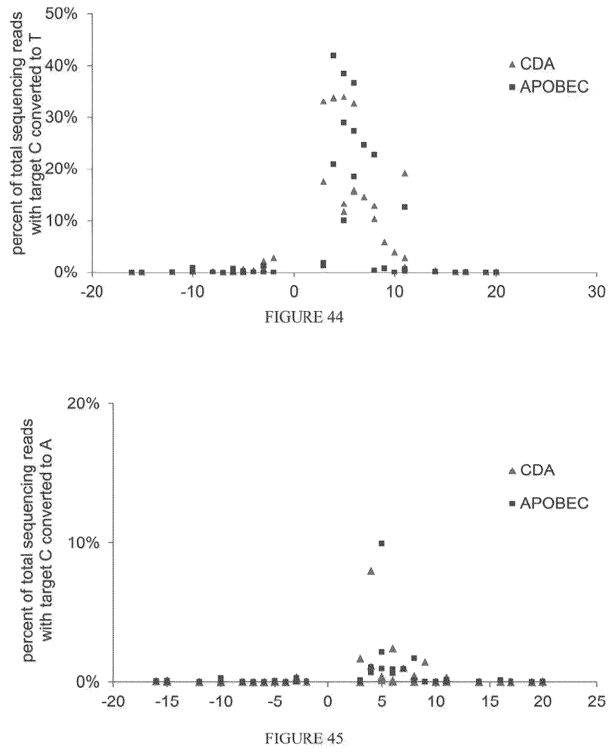

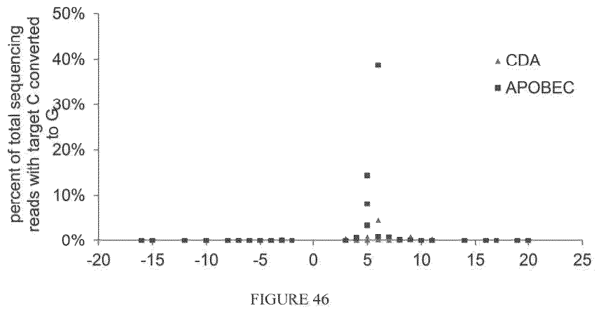

The percent of total sequencing reads with target C converted to T (FIG. 44), C converted to A (FIG. 45), and C converted to G (FIG. 46) are shown for CDA and APOBEC1 (the BE3 construct).

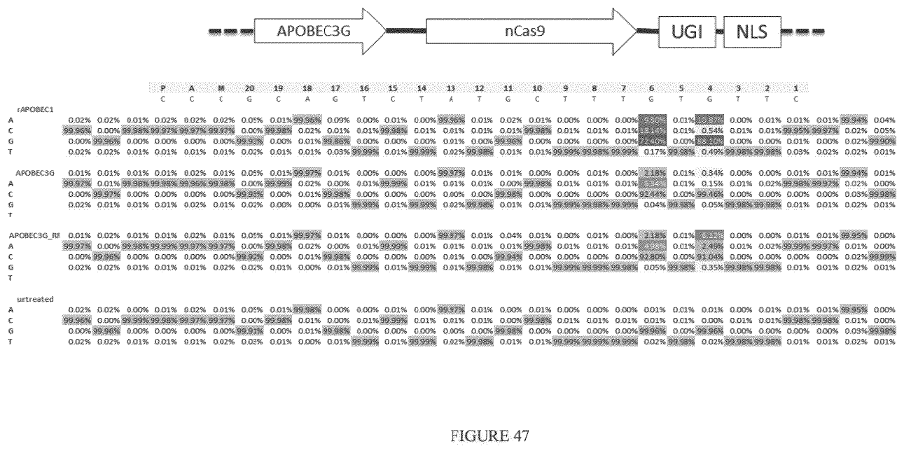

The huAPOBEC3G activity at the HeK-2 site is shown in FIG. 47. Two constructs were used: huAPOBEC3G-XTEN-nCas9-UGI-NLS and huAPOBEC3G*(D316R_D317R)-XTEN-nCas9-UGI-NLS. The huAPOBEC3G-XTEN-nCas9-UGI-NLS construct has different sequence specificity than rAPOBEC1 (BE3), as shown in FIG. 47, the editing window appears narrow, as indicated by APOBEC3G's decreased activity at position 4 compared to APOBEC1. Mutations made in huAPOBEC3G (D316R and D317R) increased ssDNA binding and resulted in an observable effect on expanding the sites which were edited (compare APOBEC3G with APOBEC3G_RR in FIG. 47). Mutations were chosen based on APOBEC3G crystal structure, see: Holden et al., Crystal structure of the anti-viral APOBEC3G catalytic domain and functional implication. Nature. (2008); 121-4, the entire contents of which are incorporated herein by reference.

### Example 7: PmCDA1/huAPOBEC3G/rAPOBEC1 Work in E. coli

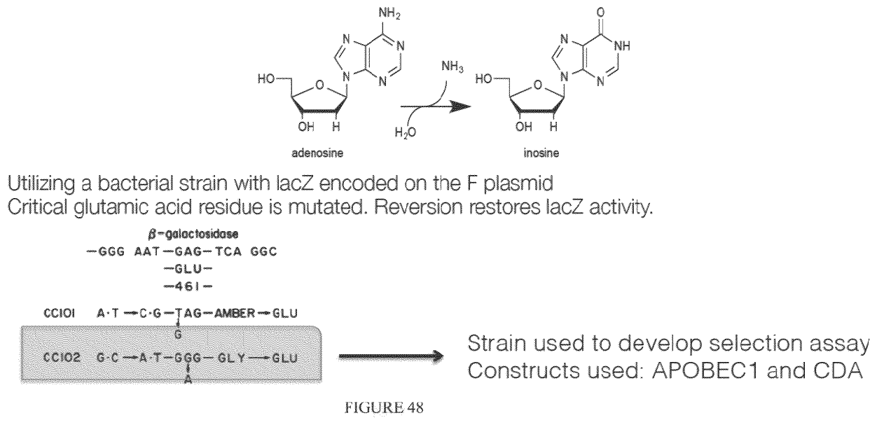

LacZ selection optimization for the A to I conversion was performed using a bacterial strain with lacZ encoded on the F plasmid. A critical glutamic acid residue was mutated (e.g., GAG to GGG, Glu to Gly mutation) so that G to A by a cytidine deaminase would restore lacZ activity (FIG. 48). Strain CC102 was selected for the selection assay. APOBEC1 and CDA constructs were used in a selection assay to optimize G to A conversion.

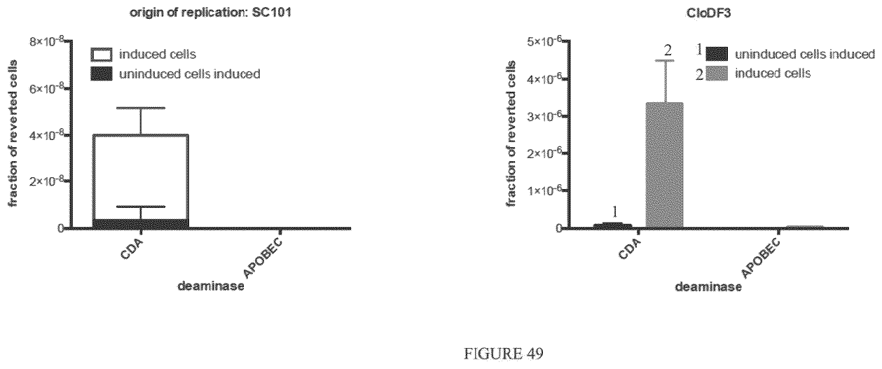

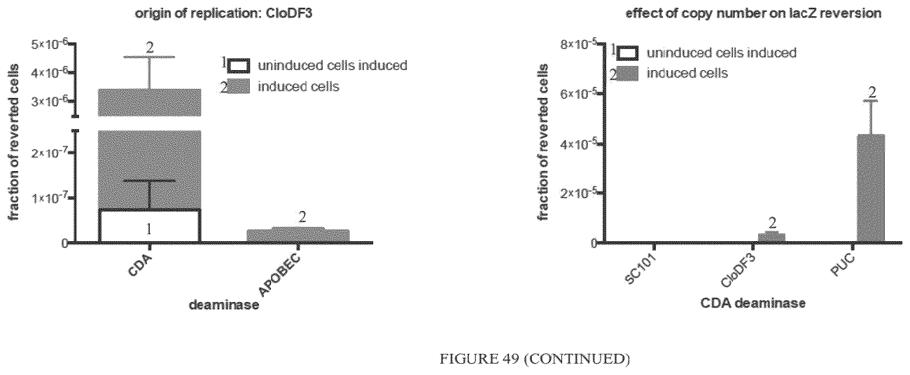

To evaluate the effect of copy number of the plasmids encoding the deaminase constructs on lacZ reversion frequency, the CDA and APOBEC1 deaminases were cloned into 4 plasmids with different replication origins (hence different copy numbers), SC101, CloDF3, RSF1030, and PUC (copy number: PUC>RSF1030>CloDF3>SC101) and placed under an inducible promoter. The plasmids were individually transformed into E. coli cells harboring F plasmid containing the mutated LacZ gene. The expression of the deaminases were induced and LacZ activity was detected for each construct (FIG. 49). As shown in FIG. 49, CDA exhibited significantly higher activity than APOBEC1 in all instances, regardless of the plasmid copy number the deaminases were cloned in. Further, In terms of the copy number, the deaminase activity was positively correlated with the copy number of the plasmid they are cloned in, i.e., PUC>CloDF3>SC101.

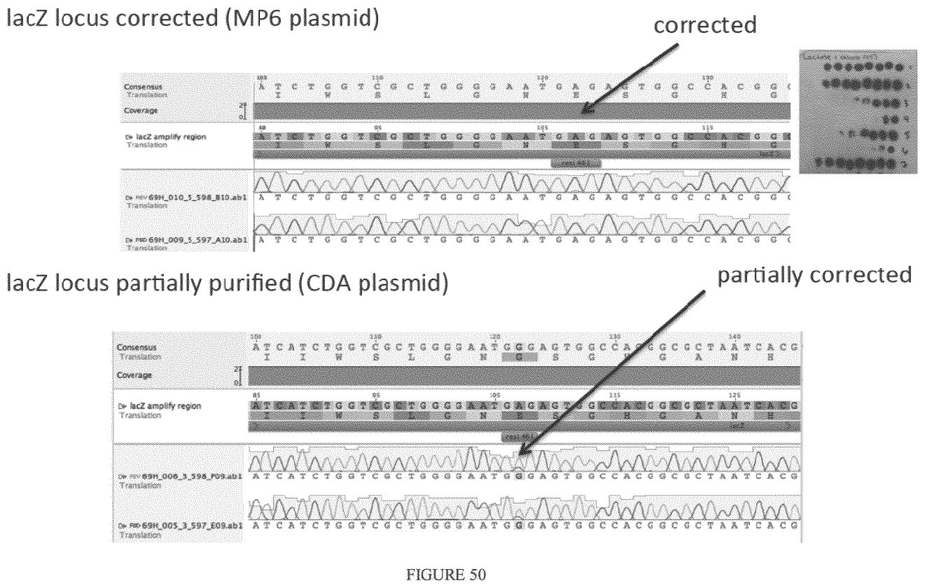

LacZ reversions were confirmed by sequencing of the genomic DNA at the lacZ locus. To obtain the genomic DNA containing the corrected LacZ gene, cells were grown media containing X-gal, where cells having LacZ activity form blue colonies. Blue colonies were selected and grown in minimal media containing lactose. The cells were spun down, washed, and re-plated on minimal media plates (lactose). The blue colony at the highest dilution was then selected, and its genomic DNA was sequenced at the lacZ locus (FIG. 50).

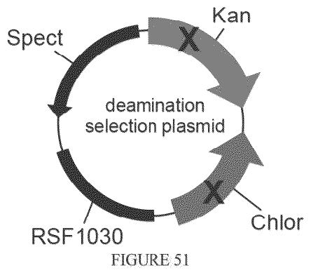

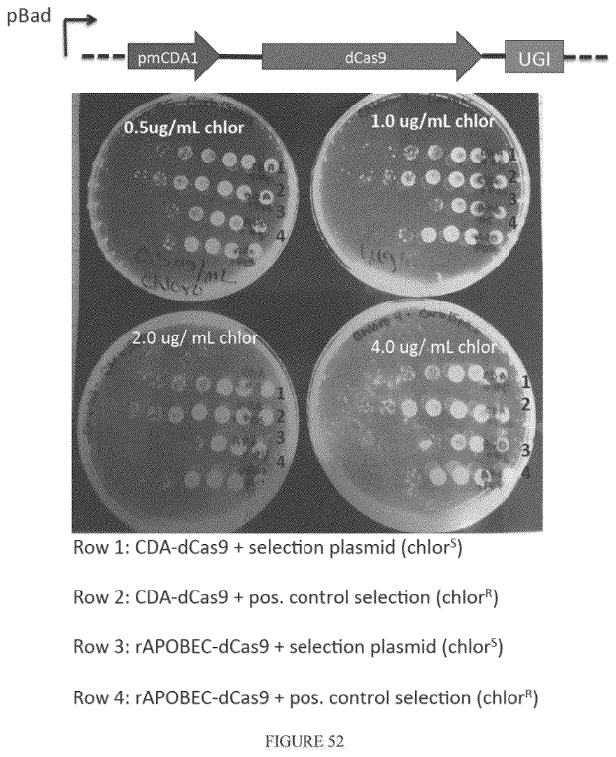

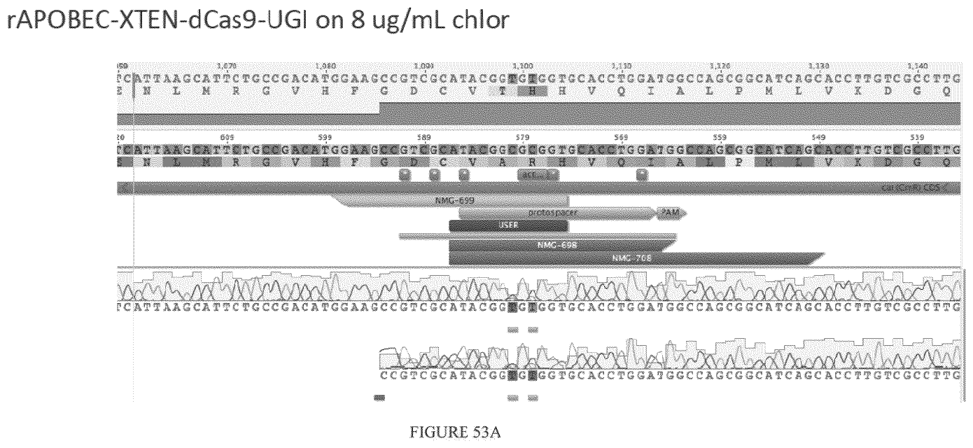

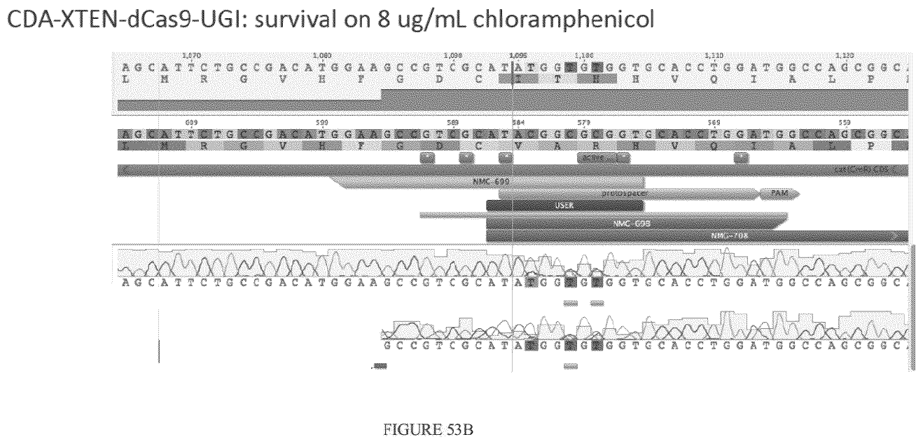

A chloramphenicol reversion assay was designed to test the activity of different cytidine deaminases (e.g., CDA, and APOBEC1). A plasmid harboring a mutant CAT1 gene which confers chloramphenicol resistance to bacteria is constructed with RSF1030 as the replication origin. The mutant CAT1 gene encodings a CAT1 protein that has a H195R (CAC to CGC) mutation, rendering the protein inactive (FIG. 51). Deamination of the C base-paired to the G base in the CGC codon would convert the codon back to a CAC codon, restoring the activity of the protein. As shown in FIG. 52, CDA outperforms rAPOBEC in E. coli in restoring the activity of the chloramphenicol resistance gene. The minimum inhibitory concentration (MIC) of chlor in S1030 with the selection plasmid (pNMG_ch_5) was approximately 1 μg/mL. Both rAPOBEC-XTEN-dCas9-UGI and CDA-XTEN-dCas9-UGI induced DNA correction on the selection plasmid (FIG. 53).

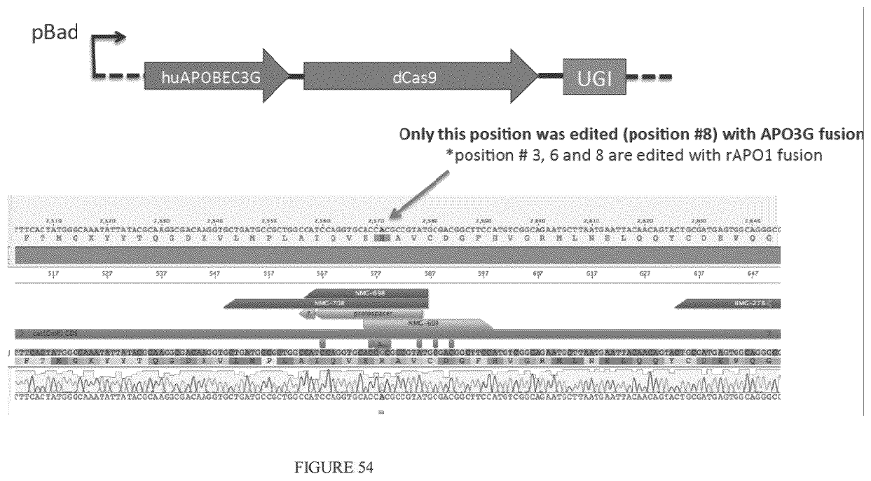

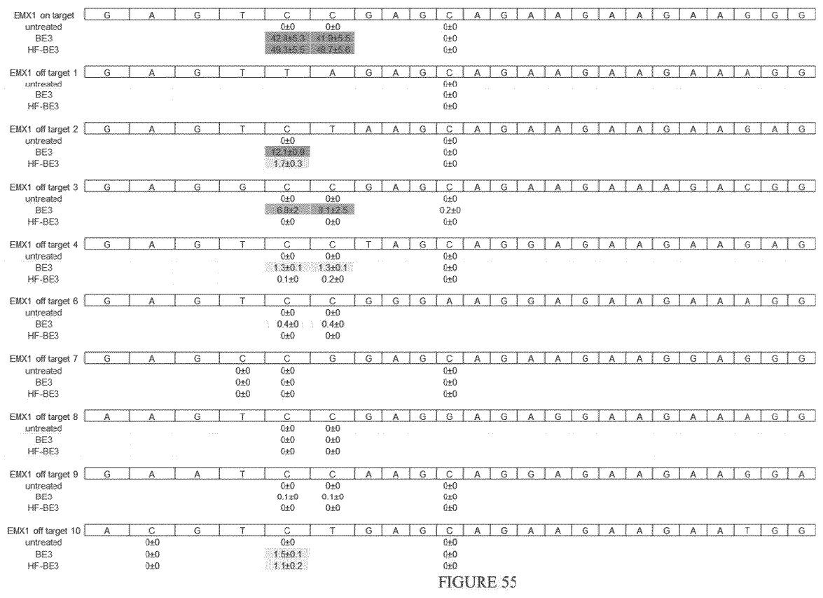

Next, the huAPOBEC3G-XTEN-dCas9-UGI protein was tested in the same assay. Interestingly, huAPOBEC3G-XTEN-dCas9-UGI exhibited different sequence specificity than the rAPOBEC1-XTEN-dCas9-UGI fusion protein. Only position 8 was edited with APOBEC3G-XTEN-dCas9-UGI fusion, as compared to the rAPOBEC11-XTEN-dCas9-UGIfusion (in which positions 3, 6, and 8 were edited) (FIG. 54).

### Example 8: C to T Base Editors with Less Off Target Editing

Current base editing technologies allow for the sequence-specific conversion of a C:G base pair into a T:A base pair in genomic DNA. This is done via the direct catalytic conversion of cytosine to uracil by a cytidine deaminase enzyme and thus, unlike traditional genome editing technologies, does not introduce double-stranded DNA breaks (DSBs) into the DNA as a first step. See, Komor, A. C., Kim, Y. B., Packer, M. S., Zuris, J. A., and Liu, D. R. (2016), “Programmable editing of a target base in genomic DNA without double-stranded DNA cleavage.” Nature 533, 420-424; the entire contents of which are incorporated by reference herein. Instead, catalytically dead SpCas9 (dCas9) or a SpCas9 nickase (dCas9(A840H)) is tethered to a cytidine deaminase enzyme such as rAPOBEC1, pmCDA1, or hAPOBEC3G. The genomic locus of interest is encoded by an sgRNA, and DNA binding and local denaturation is facilitated by the dCas9 portion of the fusion. However, just as wt dCas9 and wt Cas9 exhibit off-target DNA binding and cleavage, current base editors also exhibit C to T editing at Cas9 off-target loci, which limits their therapeutic usefulness.

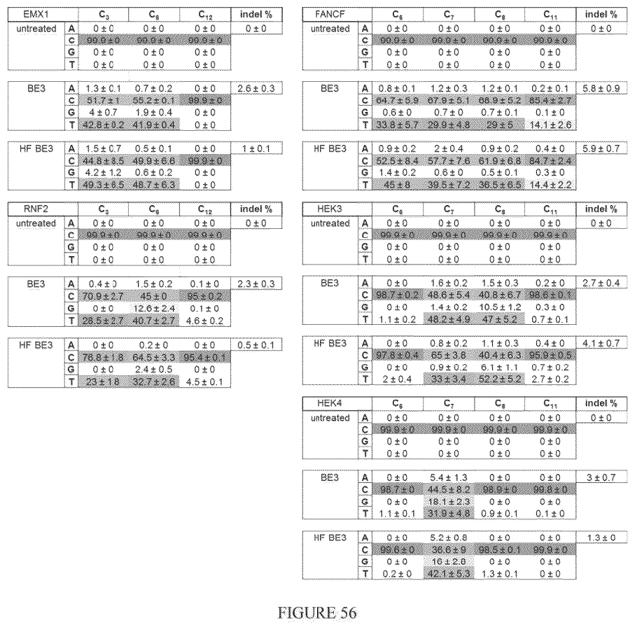

It has been reported that the introduction of just three to four mutations into SpCas9 that neutralize nonspecific electrostatic interactions between the protein and the sugar-phosphate backbone of its target DNA, increases the DNA binding specificity of SpCas9. See, Kleinstiver, B. P., Pattanayak, V., Prew, M. S., Tsai, S. Q., Nguyen, N. T., Zheng, Z., and Joung, J. K. (2016) “High-fidelity CRISPR-Cas9 nucleases with no detectable genome-wide off-target effects.” Nature 529, 490-495; and Slaymaker, I. M., Gao, L., Zetsche, B., Scott, D. A., Yan, W. X., and Zhang, F. (2015) “Rationally engineered Cas9 nucleases with improved specificity. Science 351, 84-88; the entire contents of each are hereby incorporated by reference herein. Four reported neutralizing mutations were therefore incorporated into the initially reported base editor BE3 (SEQ ID NO: 285), and found that off-target C to T editing of this enzyme is also drastically reduced (FIG. 55), with no decrease in on-target editing (FIG. 56).

As shown in FIG. 55, HEK293T cells were transfected with plasmids expressing BE3 or HF-BE3 and a sgRNA matching the EMX1 sequence using Lipofectamine 2000. Three days after transfection, genomic DNA was extracted, amplified by PCR, and analyzed by high-throughput DNA sequencing at the on-target locus, plus the top ten known Cas9 off-target loci for the EMX1 sgRNA, as previously determined by Joung and coworkers using the GUIDE-seq method. See Tsai, S. Q., Zheng, Z., Nguyen, N. T., Liebers, M., Topkar, V. V., Thapar, V., Wyvekens, N., Khayter, C., Iafrate, A. J., Le, L. P., et al. (2015) “GUIDE-seq enables genome-wide profiling of off-target cleavage by CRISPR-Cas nucleases.” Nat Biotech 33, 187-197; the entire contents of which are incorporated by reference herein. EMX1 off-target 5 locus did not amplify and is not shown. Sequences of the on-target and off-target protospacers and protospacer adjacent motifs (PAMs) are displayed (FIG. 55). Cellular C to T conversion percentages, defined as the percentage of total DNA sequencing reads with T at each position of an original C within the protospacer, are shown for BE3 and HF-BE3.

In FIG. 56, HEK293T cells were transfected with plasmids expressing BE3 or HF-BE3 and sgRNAs matching the genomic loci indicated using Lipofectamine 2000. Three days after transfection, genomic DNA was extracted, amplified by PCR, and analyzed by high-throughput DNA sequencing at the on-target loci. The percentage of total DNA sequencing reads with all four bases at the target Cs within each protospacer are shown for treatment with BE3 or HF-BE3 (FIG. 56). Frequencies of indel formation are shown as well.

### Primary Protein Sequence of HF-BE3 (SEQ ID NO: 285):

MSSETGPVAVDPTLRRRIEPHEFEVFFDPRELRKETCLLYEINWGGRHSI
WRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLKSGSETPGTSESATPESDKKYS
IGLAIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFL
VEEDKKHERHPIFGNIVDEVAYHEKYPTIYHLRKKLVDSTDKADLRLIYL
ALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSK
NGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLA
RGNSRFAWMTRKSEETITPWNFEEVVDKGASAQSFIERMTAFDKNLPNEK
VLPKHSLLYEYFTVYNELTKVKYVTEGMRKPAFLSGEQKKAIVDLLFKTN
RKVTVKQLKEDYFKKIECFDSVEISGVEDRFNASLGTYHDLLKIIKDKDF
LDNEENEDILEDIVLTLTLFEDREMIEERLKTYAHLFDDKVMKQLKRRRY
TGWGALSRKLINGIRDKQSGKTILDFLKSDGFANRNFMALIHDDSLTFKE
DIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERG
GLSELDKAGFIKRQLVETRAITKHVAQILDSRMNTKYDENDKLIREVKVI
TLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGE
IRKRPLIETNGETGEIVWDKGRDFATVRKVLSMPQVNIVKKTEVQTGGFS
KESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFEL
ENGRKRMLASAGELQKGNELALPSKYVNFLYLASHYEKLKGSPEDNEQKQ
LFVEQHKHYLDEIIEQISEFSKRVILADANLDKVLSAYNKHRDKPIREQA
ENIIHLFTLTNLGAPAAFKYFDTTIDRKRYTSTKEVLDATLIHQSITGLY
ETRIDLSQLGGDSGGSTNLSDIIEKETGKQLVIQESILMLPEEVEEVIGN
KPESDILVHTAYDESTDENVMLLTSDAPEYKPWALVIQDSNGENKIKMLS
GGSPKKKRKV

### Example 9: Development of Base Editors that Use Cas9 Variants and Modulation of the Base Editor Processivity to Increase the Target Range and Precision of the Base Editing Technology

Unlike traditional genome editing platforms, base editing technology allows precise single nucleotide changes in the DNA without inducing double-stranded breaks (DSBs). See, Komor, A. C. et al. Nature 533, 420-424 (2016). The current generation of base editor uses the NGG PAM exclusively. This limits its ability to edit desired bases within the genome, as the base editor needs to be placed at a precise location where the target base is placed within a 4-base region (the ‘deamination window’), approximately 15 bases upstream of the PAM. See, Komor, A. C. et al. Nature 533, 420-424 (2016). Moreover, due to the high processivity of cytidine deaminase, the base editor may convert all cytidines within its deamination window into thymidines, which could induce amino acid changes other than the one desired by the researcher. See, Komor, A. C. et al. Nature 533, 420-424 (2016).

Expanding the Scope of Base Editing Through the Development of Base Editors with Cas9 Variants

Cas9 homologs and other RNA-guided DNA binders that have different PAM specificities were incorporated into the base editor architecture. See, Kleinstiver, B. P. et al. Nature 523, 481-485 (2015); Kleinstiver, B. P. et al. Nature Biotechnology 33, 1293-1298 (2015); and Zetsche, B. et al. Cell 163, 759-771 (2015); the entire contents of each are incorporated by reference herein. Furthermore, innovations that have broadened the PAM specificities of various Cas9 proteins were also incorporated to expand the target reach of the base editor even more. See, Kleinstiver, B. P. et al. Nature 523, 481-485 (2015); and Kleinstiver, B. P. et al. Nature Biotechnology 33, 1293-1298 (2015). The current palette of base editors is summarized in Table 4.

TABLE 4
New base editors made from Cas9 Variants

| | | | Reference for Cas9 |
|---|---|---|---|
| Species | PAM | Base Editor Name | variant |
| S. pyogenes | . . . NGG | BE3 | Wild-type |
| | . . . NGA | VQR BE3 or EQR | Kleinstiver, B. P. et |
| | | BE3 | al. |
| | . . . NGCG | VRER BE3 | Kleinstiver, B. P. et |
| | | | al. |
| S. aureus | . . . NNGRRT | SaBE3 | Wild-type |
| | . . . NNNRRT | SaKKH BE3 | Kleinstiver, B. P. et |
| | | | al. |
| L. bacterium | TTTN . . . | dCpf1 BE2 | Zetsche, B. et al. |

Modulating Base Editor's Processivity Through Site-Directed Mutagenesis of rAPOBEC1

It was reasoned that the processivity of the base editor could be modulated by making point mutations in the deaminase enzyme. The incorporation of mutations that slightly reduce the catalytic activity of deaminase in which the base editor could still catalyze on average one round of cytidine deamination but was unlikely to access and catalyze another deamination within the relevant timescale were pursued. In effect, the resulting base editor would have a narrower deamination window.

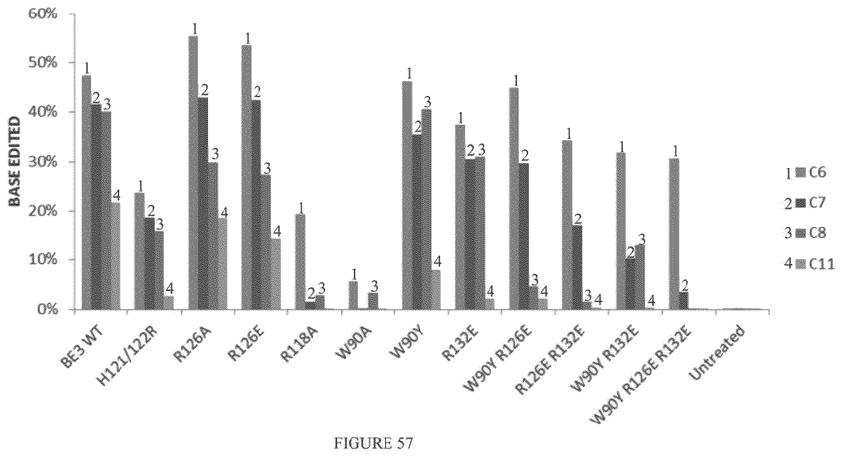

rAPOBEC1 mutations probed in this work are listed in Table 5. Some of the mutations resulted in slight apparent impairment of rAPOBEC1 catalysis, which manifested as preferential editing of one cytidine over another when multiple cytidines are found within the deamination window. Combining some of these mutations had an additive effect, allowing the base editor to discriminate substrate cytidines with higher stringency. Some of the double mutants and the triple mutant allowed selective editing of one cytidine among multiple cytidines that are right next to one another (FIG. 57).

TABLE 5
rAPOBEC1 Point Mutations Investigated

| rAPOBEC1 mutation | Corresponding mutation in | |
|---|---|---|
| studied in this work | APOBEC3G | Reference |
| H121R/H122R | D315R/D316R | Holden, L. G. et al. |
| R126A | R320A | Chen, K-M. et al. |
| R126E | R320E | Chen, K-M. et al. |
| R118A | R313A | Chen, K-M. et al. |
| W90A | W285A | Chen, K-M. et al. |
| W90Y | W285Y | |
| R312E | R326E | |

### Base Editor PAM Expansion and Processivity Modulation

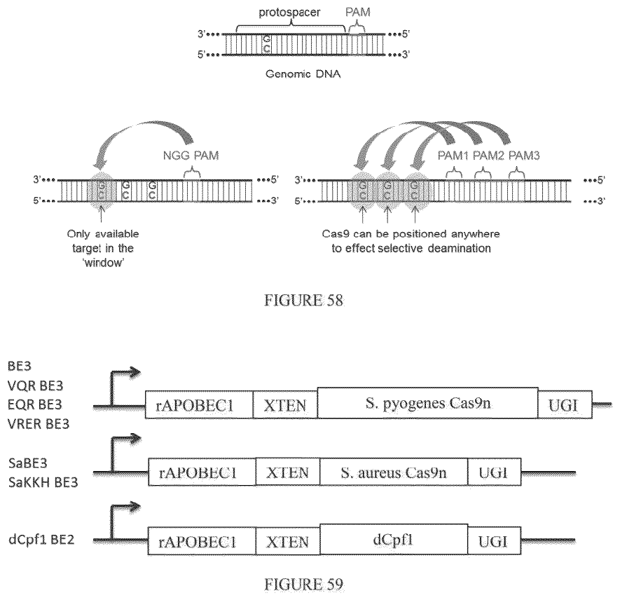

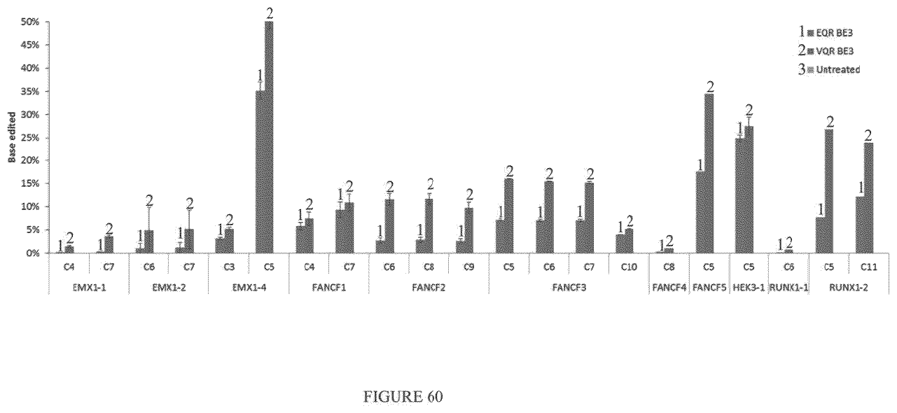

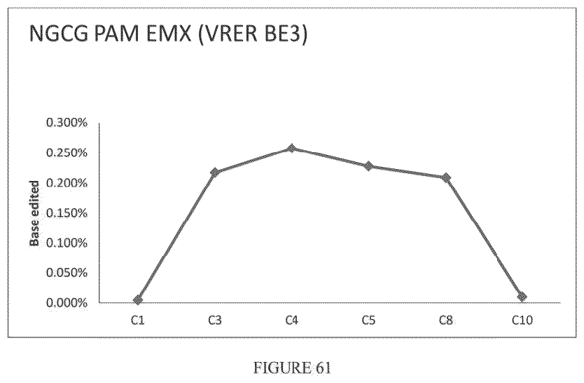

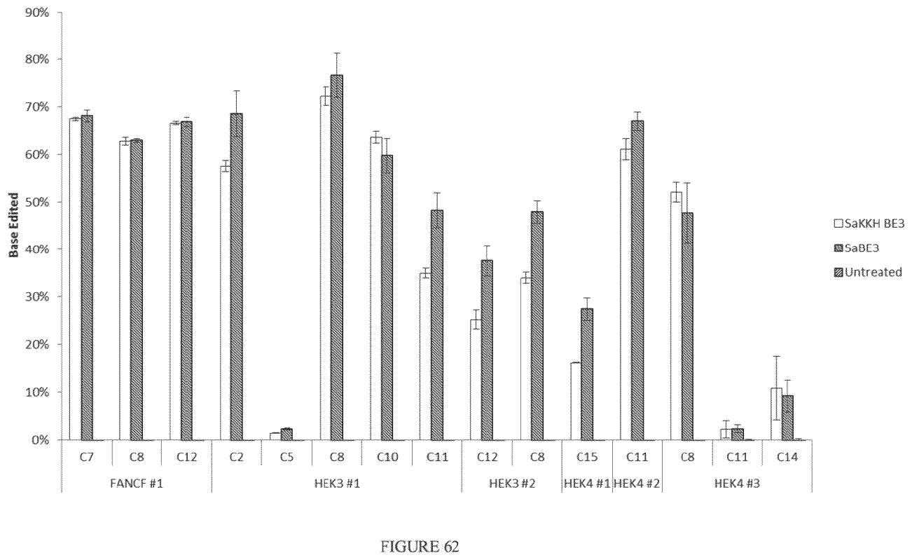

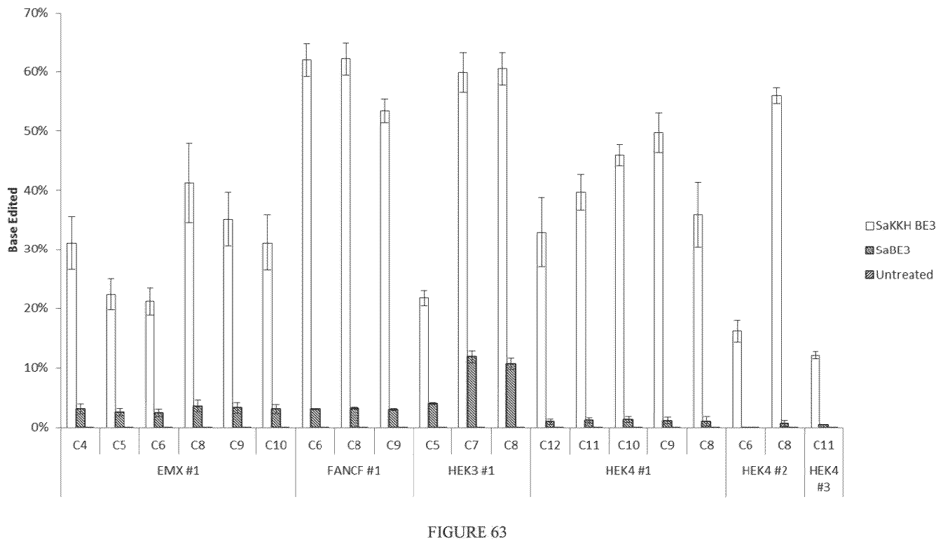

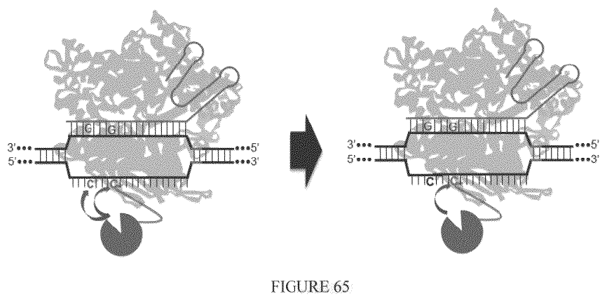

The next generation of base editors were designed to expand editable cytidines in the genome by using other RNA-guided DNA binders (FIG. 58). Using a NGG PAM only allows for a single target within the “window” whereas the use of multiple different PAMs allows for Cas9 to be positioned anywhere to effect selective deamination. A variety of new base editors have been created from Cas9 variants (FIG. 59 and Table 4). Different PAM sites (NGA, FIG. 60; NGCG, FIG. 61; NNGRRT, FIG. 62; and NNHRRT, FIG. 63) were explored. Selective deamination was successfully achieved through kinetic modulation of cytidine deaminase point mutagenesis (FIG. 65 and Table 5).

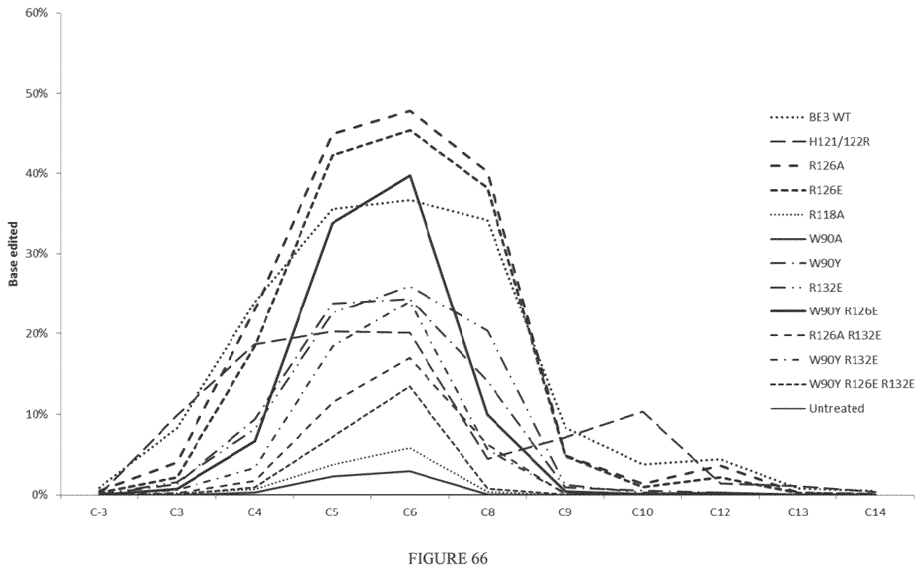

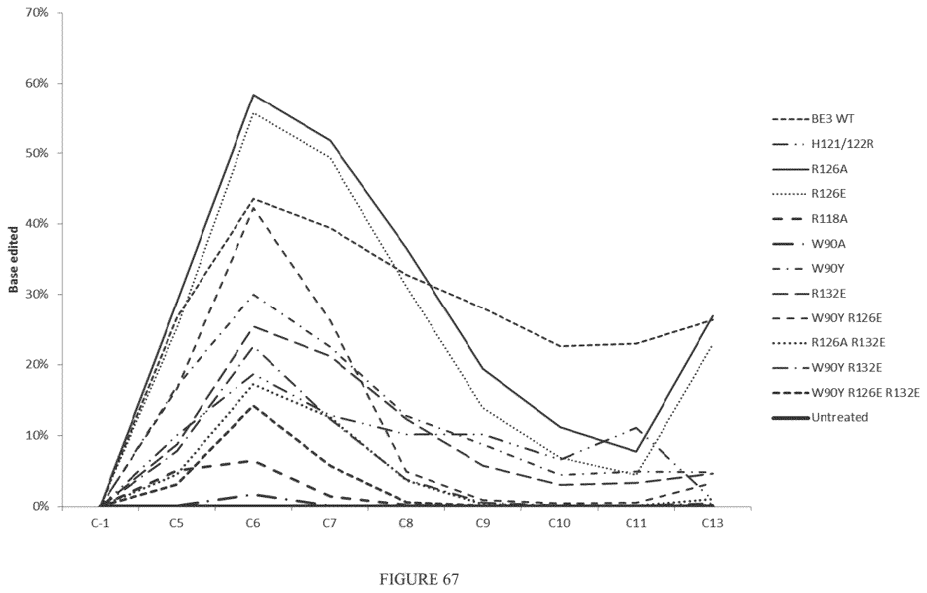

The effect of various mutations on the deamination window was then investigated in cell culture using spacers with multiple cytidines (FIGS. 66 and 67).

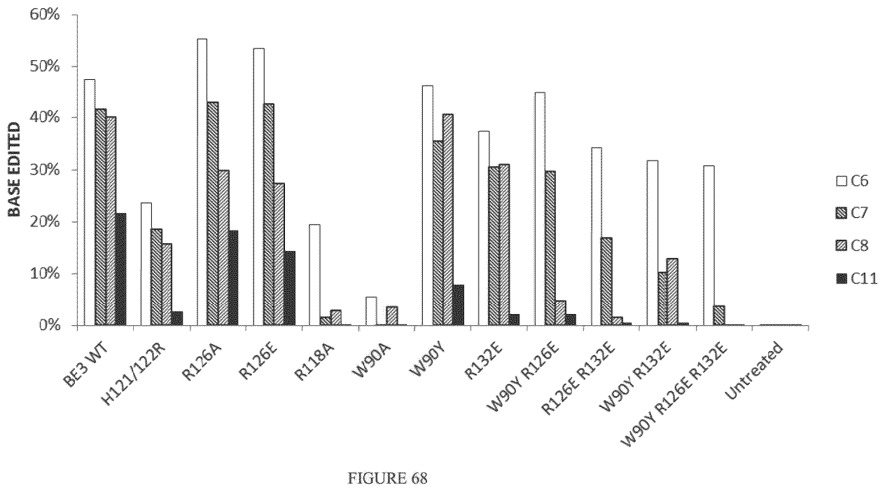

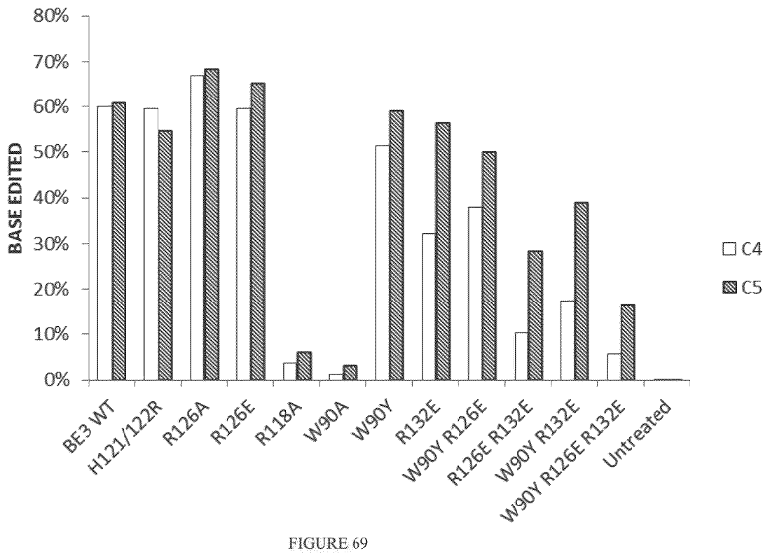

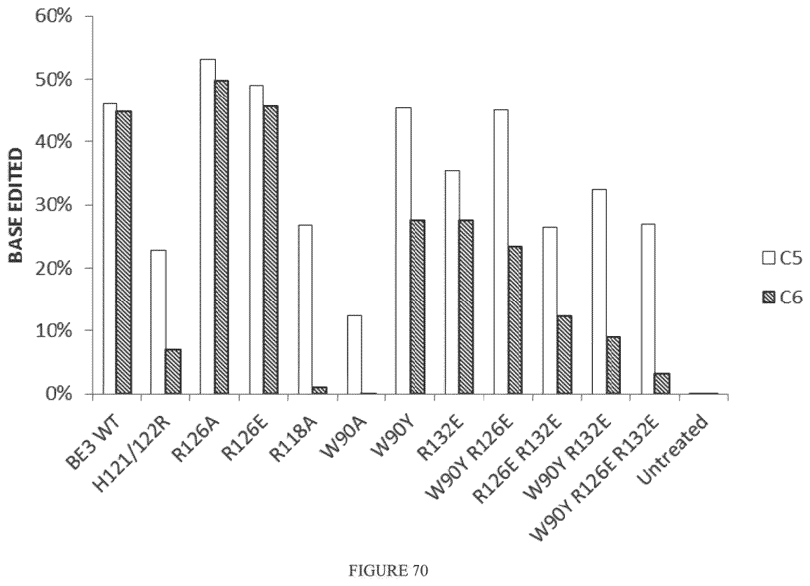

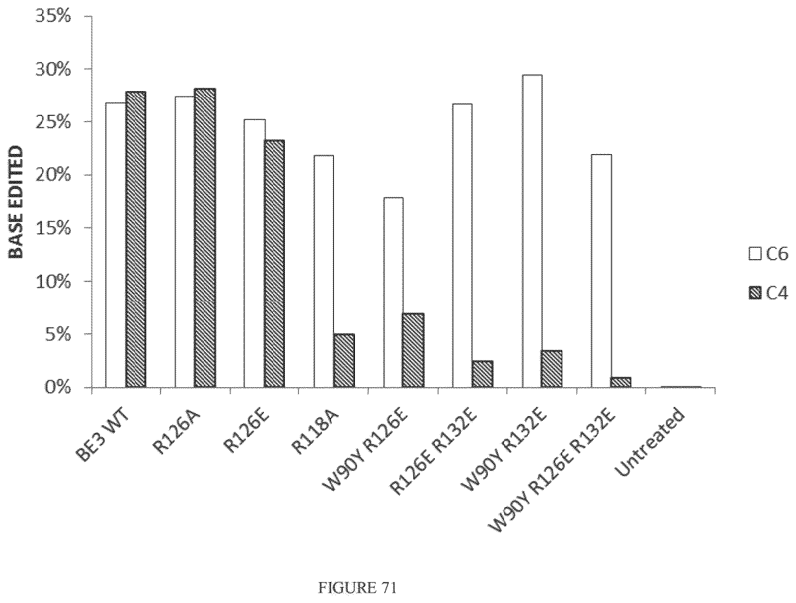

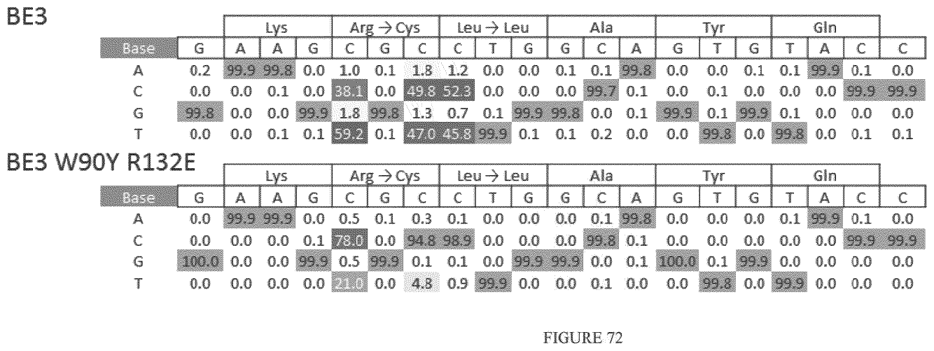

Further, the effect of various mutations on different genomic sites with limited numbers of cytidines was examined (FIGS. 68 to 71). It was found that approximately one cytidine will be edited within the deamination window in the spacer, while the rest of the cytidines will be left intact. Overall, the preference for editing is as follows: C₆>C₅>>C₇≈C₄.

### Base Editing Using Cpf1

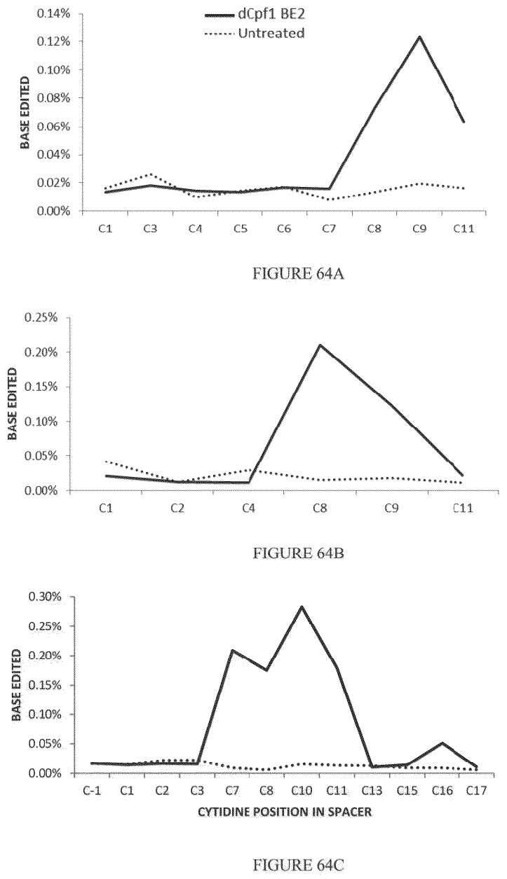

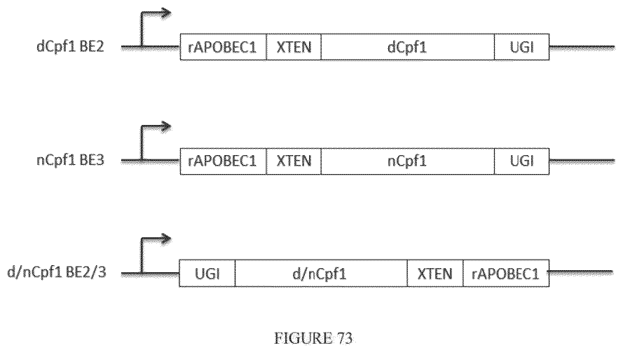

Cpf1, a Cas9 homolog, can be obtained as AsCpf1, LbCpf1, or from any other species. Schematics of fusion constructs, including BE2 and BE3 equivalents, are shown in FIG. 73. The BE2 equivalent uses catalytically inactive Cpf2 enzyme (dCpf1) instead of Cas9, while the BE3 equivalent includes the Cpf1 mutant, which nicks the target strand. The bottom schematic depicts different fusion architectures to combine the two innovations illustrated above it (FIG. 73). The base editing results of HEK293T cell TTTN PAM sites using Cpf1 BE2 were examined with different spacers (FIGS. 64A to 64C). In some embodiments, Cpf1 may be used in place of a Cas9 domain in any of the base editors provided herein. In some embodiments, the Cpf1 is a protein that is at least 50%, 55%, 60%, 65%, 70%, 75%, 80%, 85%, 90%, 95%, 98%, 99%, or 99.5% identical to SEQ ID NO 313.

### Full Protein Sequence of Cpf1 (SEQ ID NO: 313):

MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNONLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKENTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVEDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKENDEI
NLLLKEKANDVHILSIDRGERHLAYYTLVDGKGNIIKQDTFNIIGNDRMK
TNYHDKLAAIEKDRDSARKDWKKINNIKEMKEGYLSQVVHEIAKLVIEYN
AIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDADANGAYHIGLKGLMLLGRIKNNQEGKKLNLVIKNEEYFEFVQNRNN

### Example 10: Increased Fidelity of Base Editing

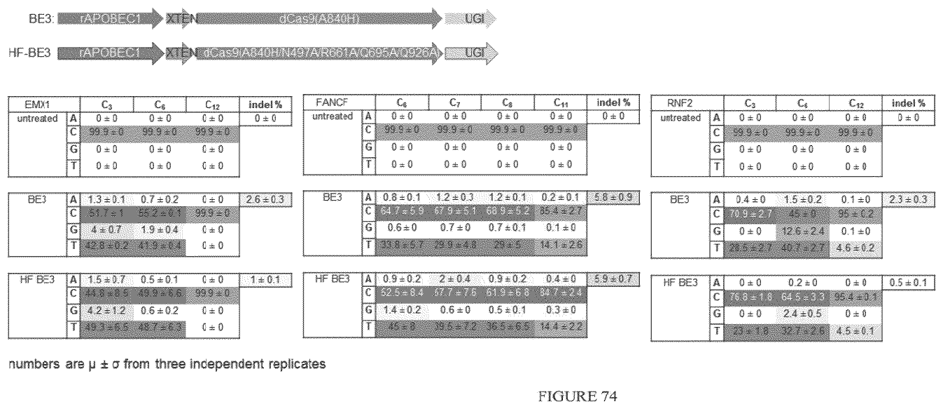

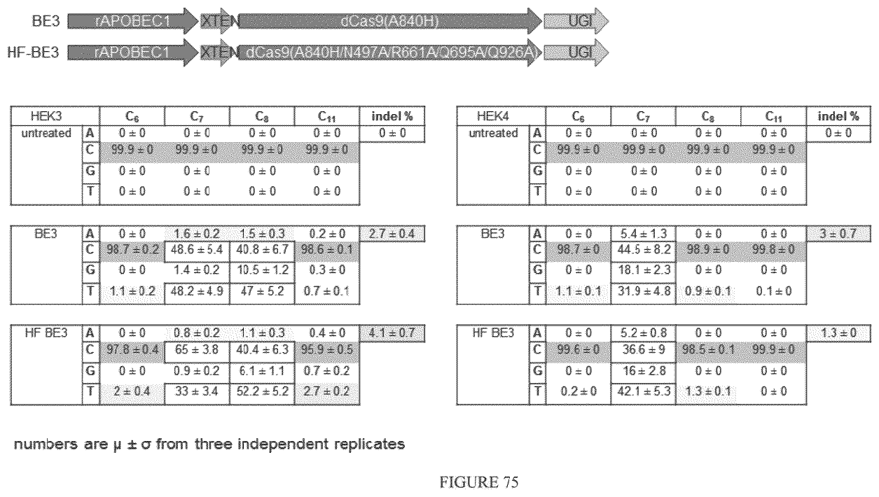

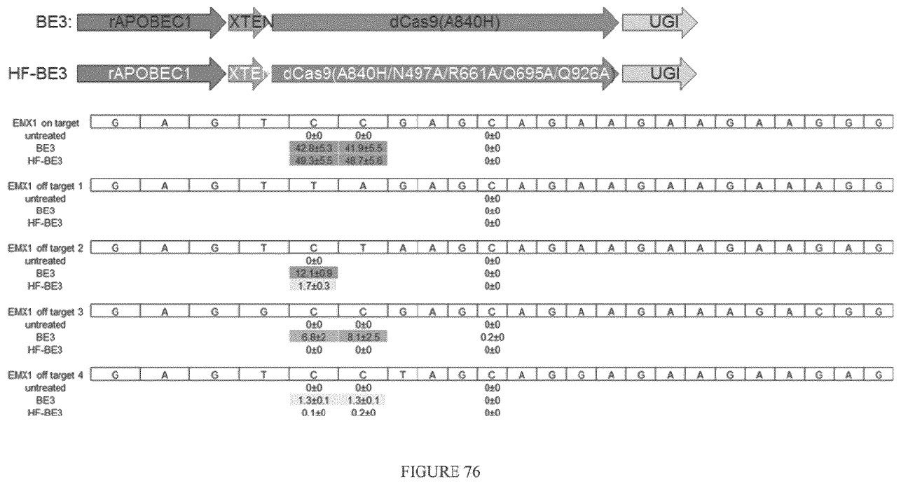

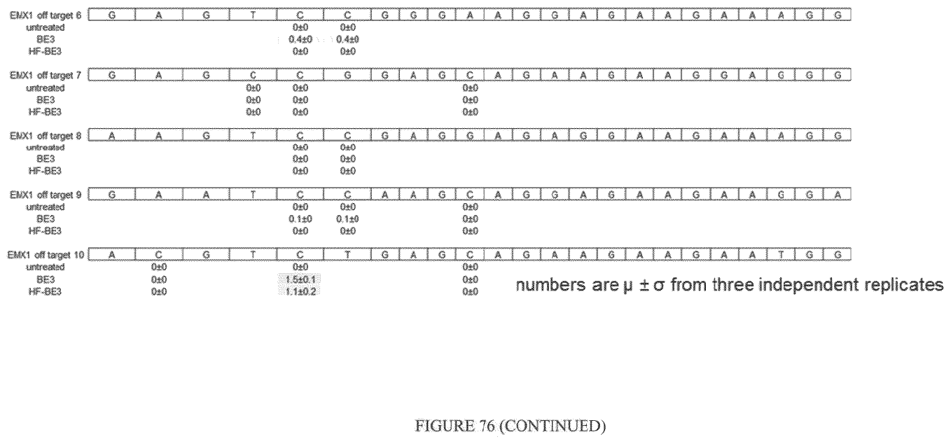

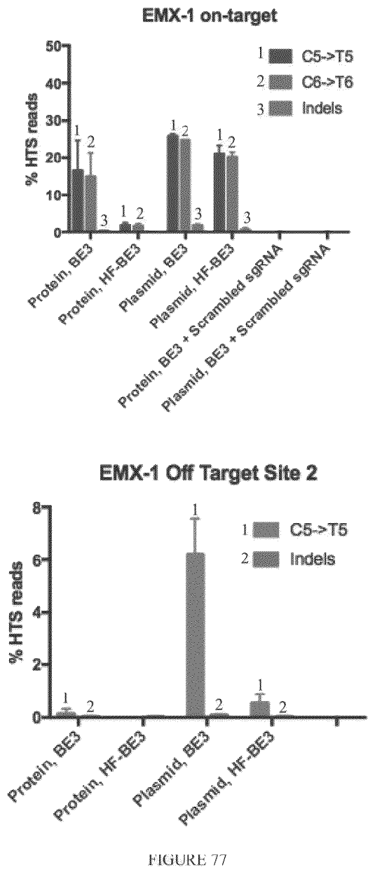

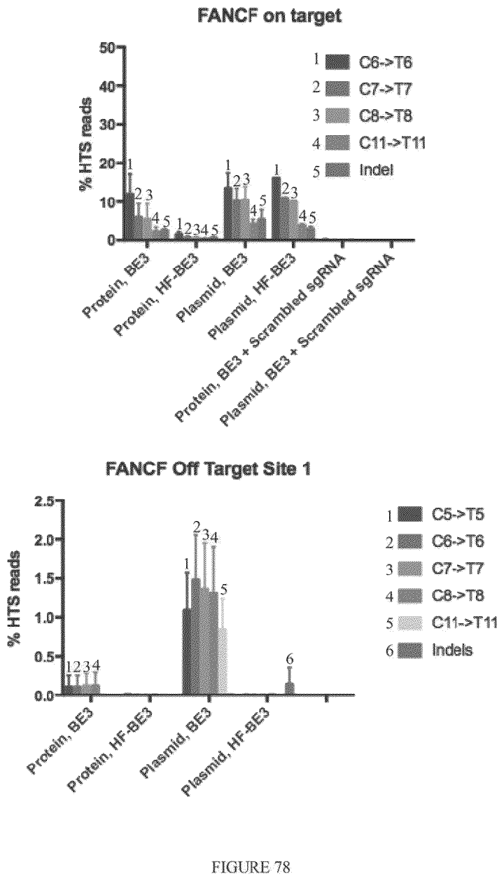

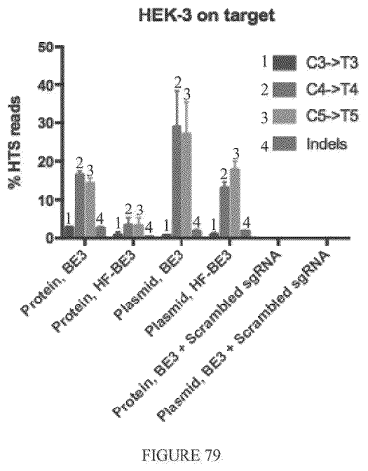

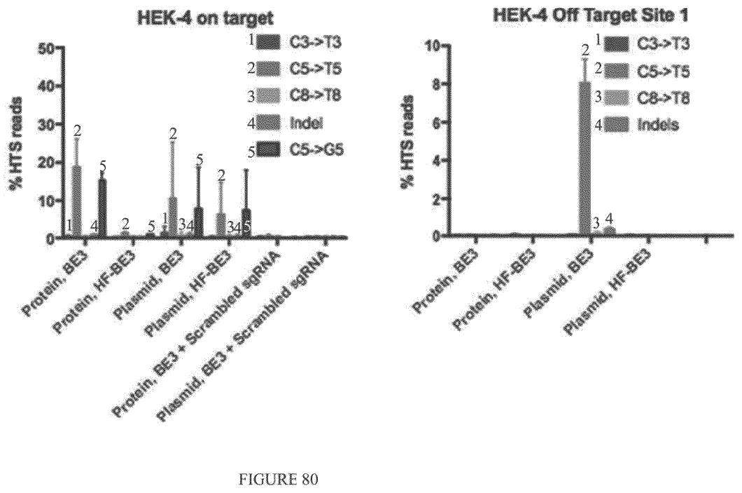

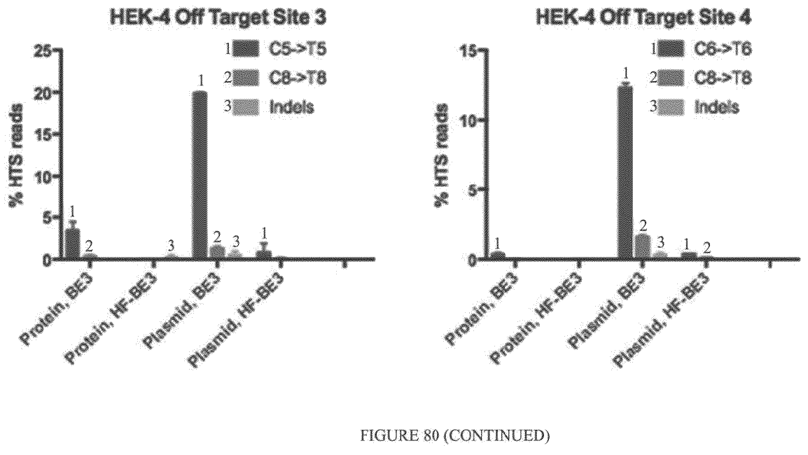

Examining the difference between plasmid delivery of BE3 and HF-BE3, it was found that the two edit on-target loci with comparable efficiency (FIGS. 74 and 75). However, HF-BE3 edited off-target loci much less than BE3, meaning that HF-BE3 has a much higher DNA specificity than BE3 (FIG. 76). Deaminase protein lipofection to HEK cells demonstrated that protein delivery of BE3 results in comparable on-target activity, but much better specificity, than plasmid DNA delivery of BE3. Using improved transfection procedures and better plasmids (n=2), the experiment used the following conditions: protein delivery was 125 nM Cas9:sgRNA complex, plasmid delivery was 750 ng BE3/HF-BE3 plasmid +250 ng sgRNA plasmid, and lipofection was with 1.5 μL of Lipofectamine 2000 per well. EMX-1 off target site 2 and FANCF off-target site 1 showed the most off-target editing with BE3, compared to all of the off-targets assayed (FIGS. 77 and 78), while HEK-3 showed no significant editing at off-targets for any of the delivery methods (FIG. 79). HEK-4 shows some C-to-G editing on at the on-target site, while its off-target sites 1, 3, and 4 showed the most off-target editing of all the assayed sites (FIG. 80).

### Delivery of BE3 Protein Via Micro-Injection to Zebrafish

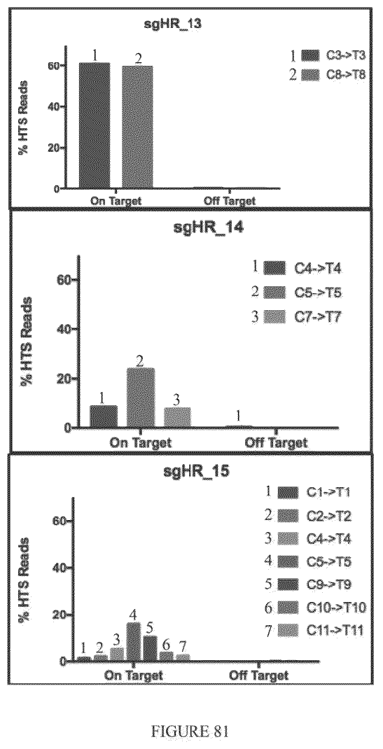

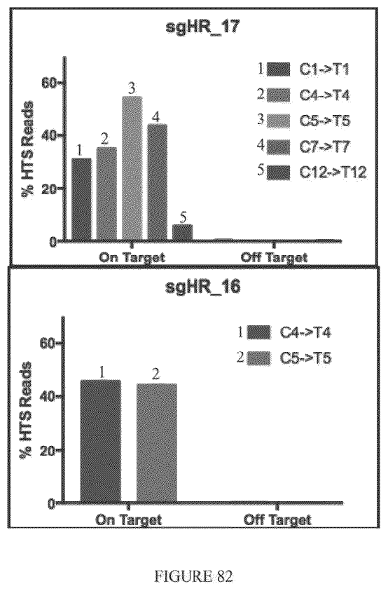

TYR guide RNAs were tested in an in vitro assay for sgRNA activity (FIGS. 81 and 82). The % HTS reads shows how many C residues were converted to T residues during a 2h incubation with purified BE3 protein and PCR of the resulting product. Experiments used an 80-mer synthetic DNA substrate with the target deamination site in 60bp of its genomic context. This is not the same as % edited DNA strands because only one strand was nicked, so the product is not amplified by PCR. The proportion of HTS reads edited is equal to x/(2−x), where x is the actual proportion of THS reads edited. For 60% editing, the actual proportion of bases edited is 75%. “Off target” is represents BE3 incubated with the same DNA substrate, while bound to an off-target sgRNA. It was found sgRNAs sgRH_13, sgHR_17, and possibly sgHR_16 appeared to be promising targets for in vivo injection experiments.

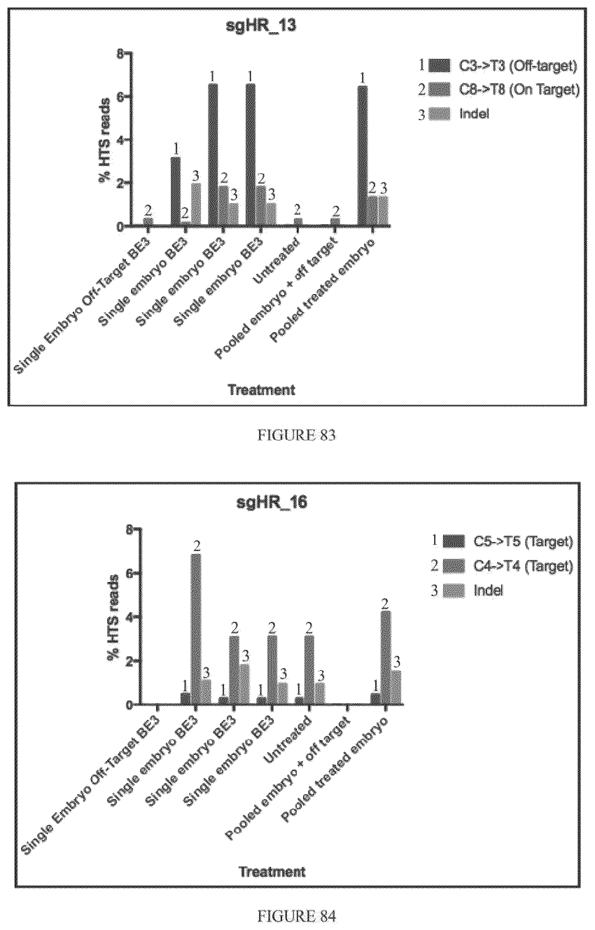

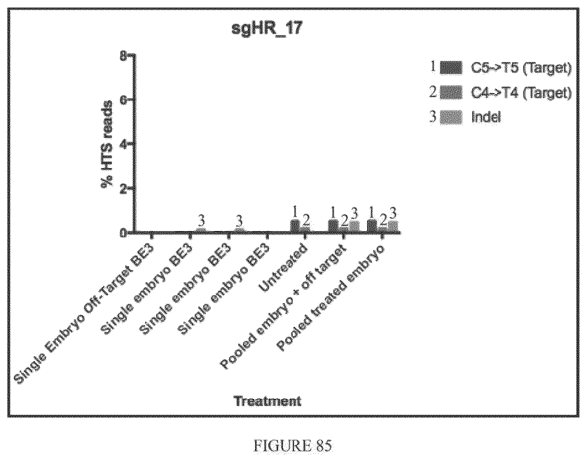

The delivery of BE3 protein in was tested in vivo in zebrafish. Zebrafish embryos (n=16-24) were injected with either scrambled sgRNA, sgHR_13, sgHR_16, or sgHR_17 and purified BE3. Three embryos from each condition were analyzed independently (single embryo) and for each condition, all of the injected embryos were pooled and sequenced as a pool. The results are shown in FIGS. 83 to 85.

### Example 11: Uses of Base Editors to Treat Disease

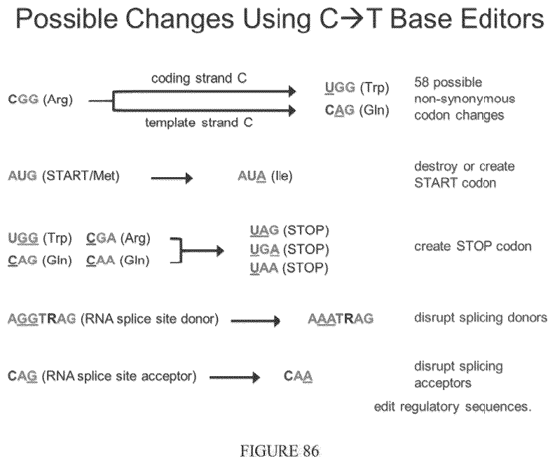

Base editors or complexes provided herein (e.g., BE3) may be used to modify nucleic acids. For example, base editors may be used to change a cytosine to a thymine in a nucleic acid (e.g., DNA). Such changes may be made to, inter alia, alter the amino acid sequence of a protein, to destroy or create a start codon, to create a stop codon, to disrupt splicing donors, to disrupt splicing acceptors or edit regulatory sequences. Examples of possible nucleotide changes are shown in FIG. 86.

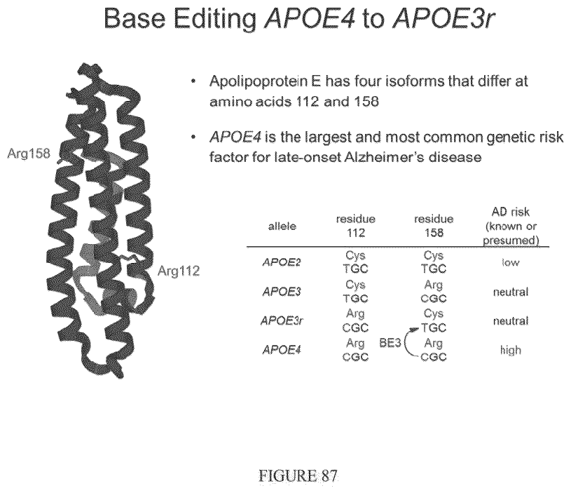

Base editors or complexes provided herein (e.g., BE3) may be used to edit an isoform of Apolipoprotein E in a subject. For example, an Apolipoprotein E isoform may be edited to yield an isoform associated with a lower risk of developing Alzheimer's disease. Apolipoprotein E has four isoforms that differ at amino acids 112 and 158. APOE4 is the largest and most common genetic risk factor for late-onset Alzheimer's disease. Arginine residue 158 of APOE4, encoded by the nucleic acid sequence CGC, may be changed to a cysteine by using a base editor (e.g., BE3) to change the CGC nucleic acid sequence to TGC, which encodes cysteine at residue 158. This change yields an APOE3r isoform, which is associated with lower Alzheimer's disease risk. See FIG. 87.

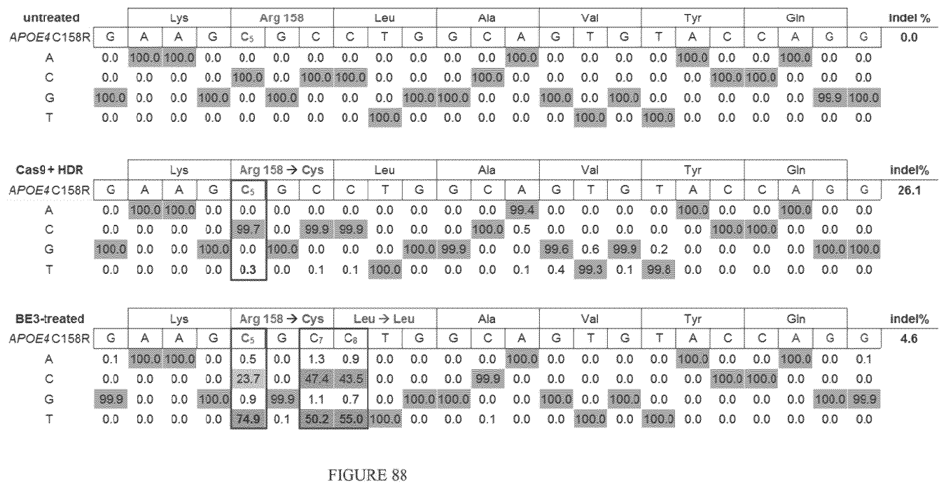

It was tested whether base editor BE3 could be used to edit APOE4 to APOE3r in mouse astrocytes (FIG. 88). APOE 4 mouse astrocytes were nucleofected with Cas9+template or BE3, targeting the nucleic acid encoding Arginine 158 of APOE4. The Cas9+template yielded only 0.3% editing with 26% indels, while BE3 yielded 75% editing with 5% indels. Two additional base-edited cytosines are silent and do not yield changes to the amino acid sequence (FIG. 88).

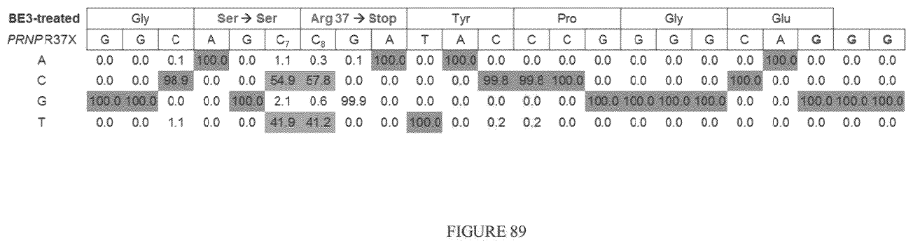

Base editors or complexes provided herein may be used to treat prion protein diseases such as Creutzfeldt-Jakob disease and fatal familial insomnia, for example, by introducing mutations into a PRNP gene. Reverting PRNP mutations may not yield therapeutic results, and intels in PRNP may be pathogenic. Accordingly, it was tested whether PRNP could be mutated using base editors (e.g., BE3) to introduce a premature stop codon in the PRNP gene. BE3, associated with its guide RNA, was introduced into HEK cells or glioblastoma cells and was capable of editing the PRNP gene to change the encoded arginine at residue 37 to a stop codon. BE3 yielded 41% editing (FIG. 89).

Additional genes that may be edited include the following: APOE editing of Arg 112 and Arg 158 to treat increased Alzheimer's risk; APP editing of Ala 673 to decrease Alzheimer's risk; PRNP editing of Arg 37 to treat fatal familial insomnia and other prion protein diseases; DMD editing of the exons 23 and 51 splice sites to treat Duchenne muscular dystrophy; FTO editing of intron 1 to treat obesity risk; PDS editing of exon 8 to treat Pendred syndrome (genetic deafness); TMC1 editing of exon 8 to treat congenital hearing loss; CYBB editing of various patient-relevant mutations to treat chronic granulomatous disease. Additional diseases that may be treated using the base editors provided herein are shown in Table 6, below.

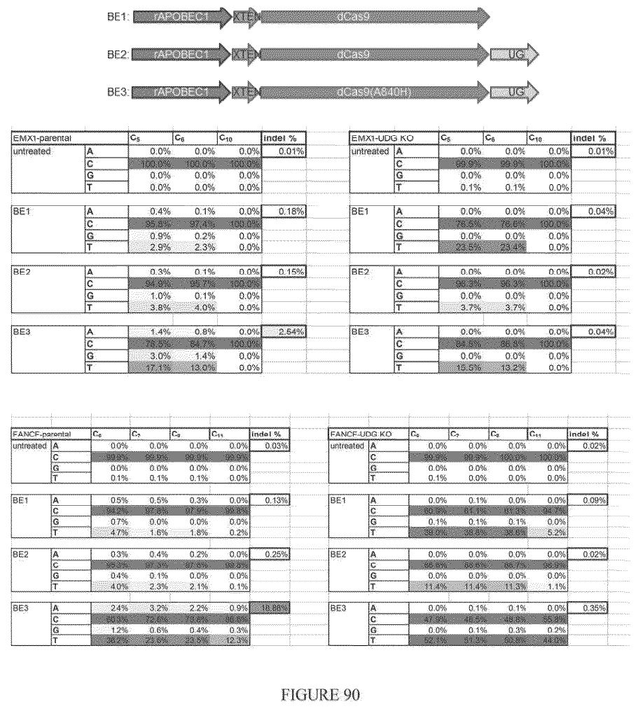

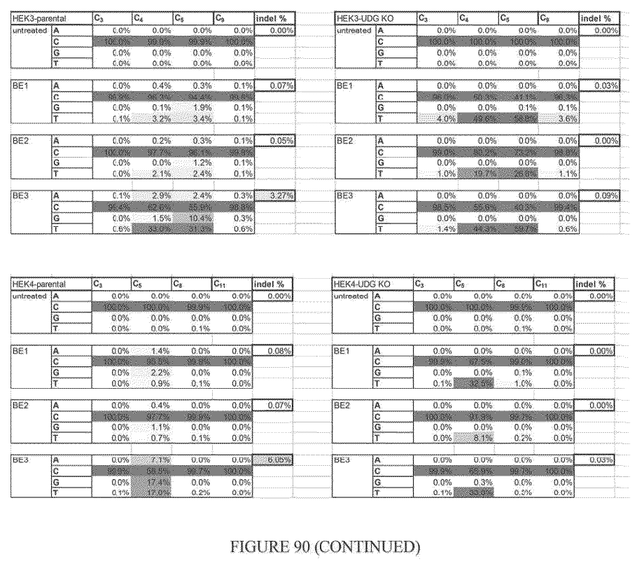

UGI also plays a key role. Knocking out UDG (which UGI inhibits) was shown to dramatically improve the cleanliness and efficiency of C to T base editing (FIG. 90).

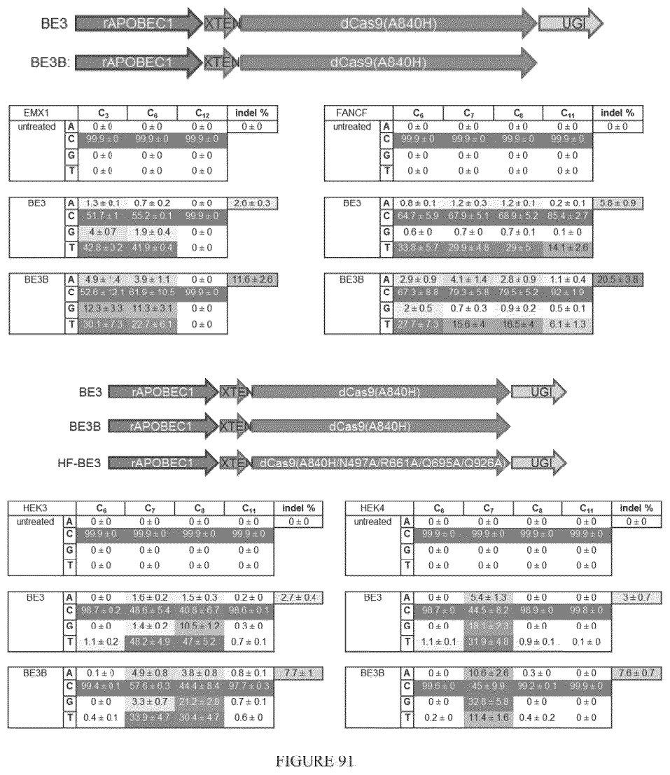

Furthermore, base editors with nickase and without UGI were shown to produce a mixture of outcomes, with very high indel rates (FIG. 91).

### Example 12: Expanding the Targeting Scope of Base Editing

Base editing is a new approach to genome editing that uses a fusion protein containing a catalytically defective Streptococcus pyogenes Cas9, a cytidine deaminase, and an inhibitor of base excision repair to induce programmable, single-nucleotide C→T (or G→A) changes in DNA without generating double-strand DNA breaks, without requiring a donor DNA template, and without inducing an excess of stochastic insertions and deletions¹. The development of five new C→T (or G→A) base editors that use natural and engineered Cas9 variants with different protospacer-adjacent motif (PAM) specificities to expand the number of sites that can be targeted by base editing by 2.5-fold are described herein. Additionally, new base editors containing mutated cytidine deaminase domains that narrow the width of the apparent editing window from approximately 5 nucleotides to 1 or 2 nucleotides were engineered, enabling the discrimination of neighboring C nucleotides that would previously be edited with comparable efficiency. Together, these developments substantially increase the targeting scope of base editing.

CRISPR-Cas9 nucleases have been widely used to mediate targeted genome editing². In most genome editing applications, Cas9 forms a complex with a single guide RNA (sgRNA) and induces a double-stranded DNA break (DSB) at the target site specified by the sgRNA sequence. Cells primarily respond to this DSB through the non-homologous end-joining (NHEJ) repair pathway, which results in stochastic insertions or deletions (indels) that can cause frameshift mutations that disrupt the gene. In the presence of a donor DNA template with a high degree of homology to the sequences flanking the DSB, gene correction can be achieved through an alternative pathway known as homology directed repair (HDR).^{3,4} Unfortunately, under most non-perturbative conditions HDR is inefficient, dependent on cell state and cell type, and dominated by a larger frequency of indels.^{3,4} As most of the known genetic variations associated with human disease are point mutations⁵, methods that can more efficiently and cleanly make precise point mutations are needed.

Base editing, which enables targeted replacement of a C:G base pair with a T:A base pair in a programmable manner without inducing DSBs¹, has been recently described. Base editing uses a fusion protein between a catalytically inactivated (dCas9) or nickase form of Streptococcus pyogenes Cas9 (SpCas9), a cytidine deaminase such as APOBEC1, and an inhibitor of base excision repair such as uracil glycosylase inhibitor (UGI) to convert cytidines into uridines within a five-nucleotide window specified by the sgRNA.¹ The third-generation base editor, BE3, converts C:G base pairs to T:A base pairs, including disease-relevant point mutations, in a variety of cell lines with higher efficiency and lower indel frequency than what can be achieved using other genome editing methods¹. Subsequent studies have validated the deaminase-dCas9 fusion approach in a variety of settings^{6,7.}

Efficient editing by BE3 requires the presence of an NGG PAM that places the target C within a five-nucleotide window near the PAM-distal end of the protospacer (positions 4-8, counting the PAM as positions 21-23)¹. This PAM requirement substantially limits the number of sites in the human genome that can be efficiently targeted by BE3, as many sites of interest lack an NGG 13- to 17-nucleotides downstream of the target C. Moreover, the high activity and processivity of BE3 results in conversion of all Cs within the editing window to Ts, which can potentially introduce undesired changes to the target locus. Herein, new C:G to T:A base editors that address both of these limitations are described.

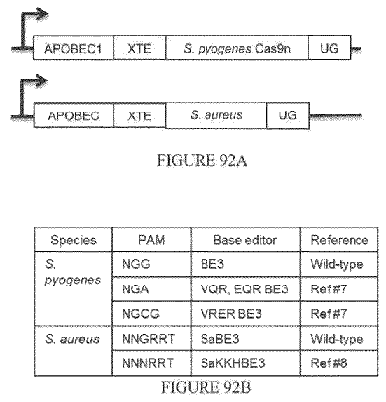

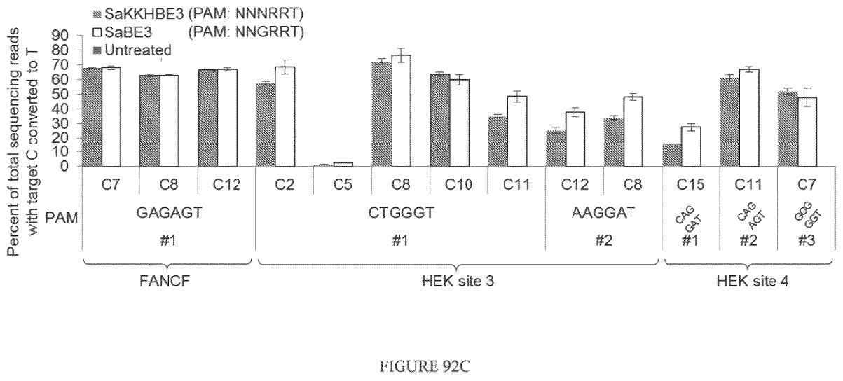

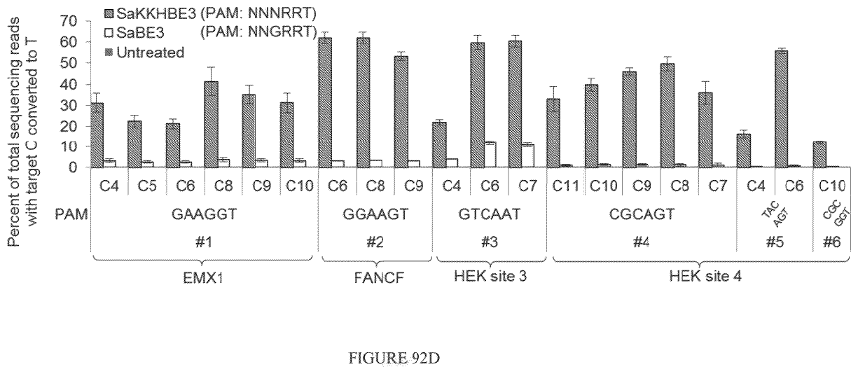

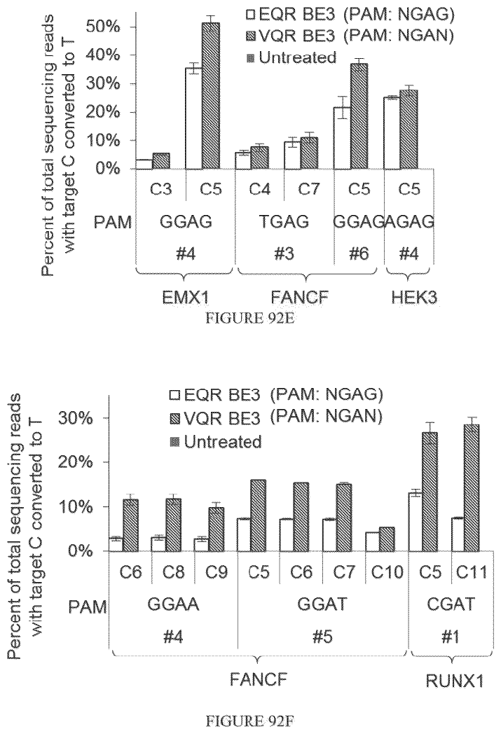

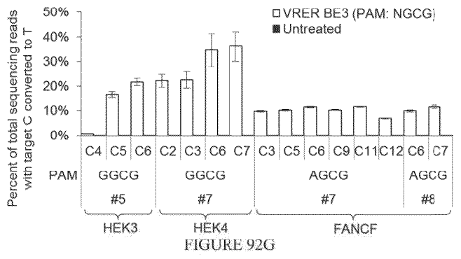

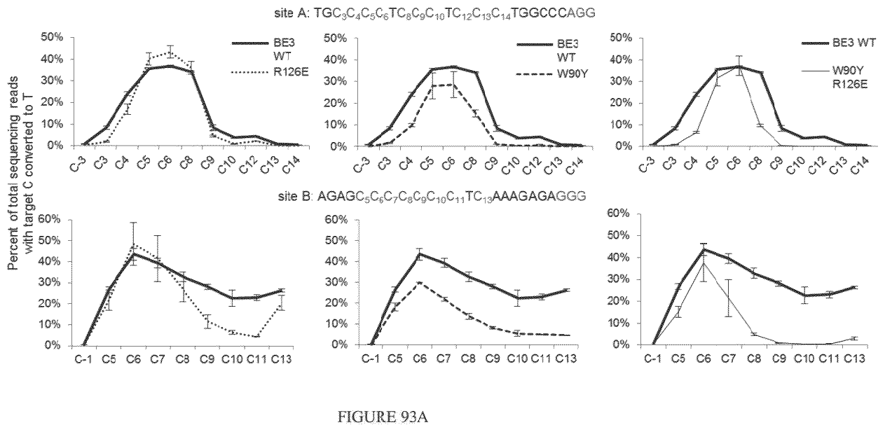

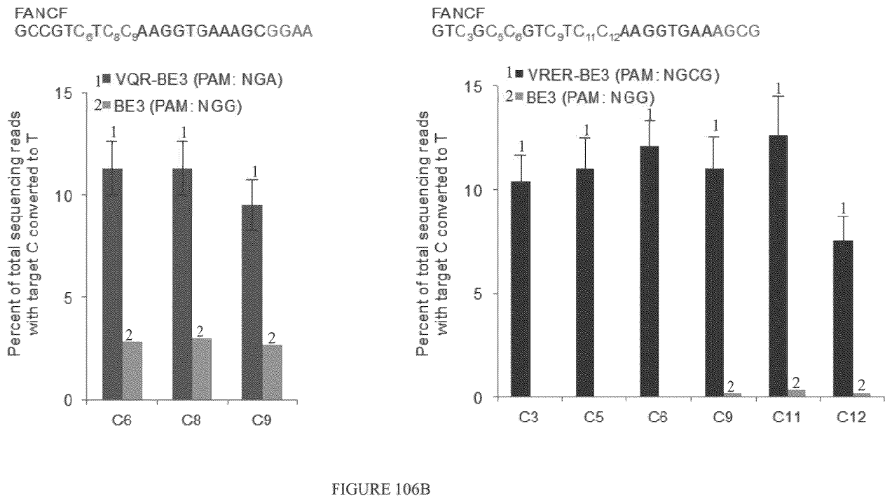

It was thought that any Cas9 homolog that binds DNA and forms an “R-loop” complex⁸ containing a single-stranded DNA bubble could in principle be converted into a base editor. These new base editors would expand the number of targetable loci by allowing non-NGG PAM sites to be edited. The Cas9 homolog from Staphylococcus aureus (SaCas9) is considerably smaller than SpCas9 (1053 vs. 1368 residues), can mediate efficient genome editing in mammalian cells, and requires an NNGRRT PAM⁹. SpCas9 was replaced with SaCas9 in BE3 to generate SaBE3 and transfected HEK293T cells with plasmids encoding SaBE3 and sgRNAs targeting six human genomic loci (FIGS. 92A and 92B). After 3 d, the genomic loci were subjected to high-throughput DNA sequencing (HTS) to quantify base editing efficiency. SaBE3 enabled C to T base editing of target Cs at a variety of genomic sites in human cells, with very high conversion efficiencies (approximately 50-75% of total DNA sequences converted from C to T, without enrichment for transfected cells) arising from targeting Cs at positions 6-11. The efficiency of SaBE3 on NNGRRT-containing target sites in general exceeded that of BE3 on NGG-containing target sites¹. Perhaps due to its higher average efficiency, SaBE3 can also result in detectable base editing at target Cs at positions outside of the canonical BE3 activity window (FIG. 92C). In comparison, BE3 showed significantly reduced editing under the same conditions (0-11%), in accordance with the known SpCas9 PAM preference (FIG. 106A)¹⁰. These data show that SaBE3 can facilitate very efficient base editing at sites not accessible to BE3.

The targeting range of base editors was further expanded by applying recently engineered Cas9 variants that expand or alter PAM specificities. Joung and coworkers recently reported three SpCas9 mutants that accept NGA (VQR-Cas9), NGAG (EQR-Cas9), or NGCG (VRER-Cas9) PAM sequences¹¹. In addition, Joung and coworkers engineered a SaCas9 variant containing three mutations (SaKKH-Cas9) that relax its PAM requirement to NNNRRT¹². The SpCas9 portion of BE3 was replaced with these four Cas9 variants to produce VQR-BE3, EQR-BE3, VRER-BE3, and SaKKH-BE3, which target NNNRRT, NGA, NGAG, and NGCG PAMs respectively. HEK293T cells were transfected with plasmids encoding these constructs and sgRNAs targeting six genomic loci for each new base editor, and measured C to T base conversions using HTS.

SaKKH-BE3 edited sites with NNNRRT PAMs with efficiencies up to 62% of treated, non-enriched cells (FIG. 92D). As expected, SaBE3 was unable to efficiently edit targets containing PAMs that were NNNHRRT (where H=A, C, or T) (FIG. 92D). VQR-BE3, EQR-BE3, and VRER-BE3 exhibited more modest, but still substantial base editing efficiencies of up to 50% of treated, non-enriched cells at genomic loci with the expected PAM requirements with an editing window similar to that of BE3 (FIGS. 92E and 92F). Base editing efficiencies of VQR-BE3, EQR-BE3, and VRER-BE3 in general closely paralleled the reported PAM requirements of the corresponding Cas9 nucleases; for example, EQR-BE3 was unable to efficiently edit targets containing NGAH PAM sequences (FIG. 92F). In contrast, BE3 was unable to edit sites with NGA or NGCG PAMs efficiently (0-3%), likely due to its PAM restrictions (FIG. 106B).

Collectively, the properties of SaBE3, SaKKH-BE3, VQR-BE3, EQR-BE3, and VRER-BE3 establish that base editors exhibit a modularity that facilitates their ability to exploit Cas9 homologs and engineered variants.

Next, base editors with altered activity window widths were developed. All Cs within the activity window of BE3 can be efficiently converted to Ts¹. The ability to modulate the width of this window would be useful in cases in which it is important to edit only a subset of Cs present in the BE3 activity window.

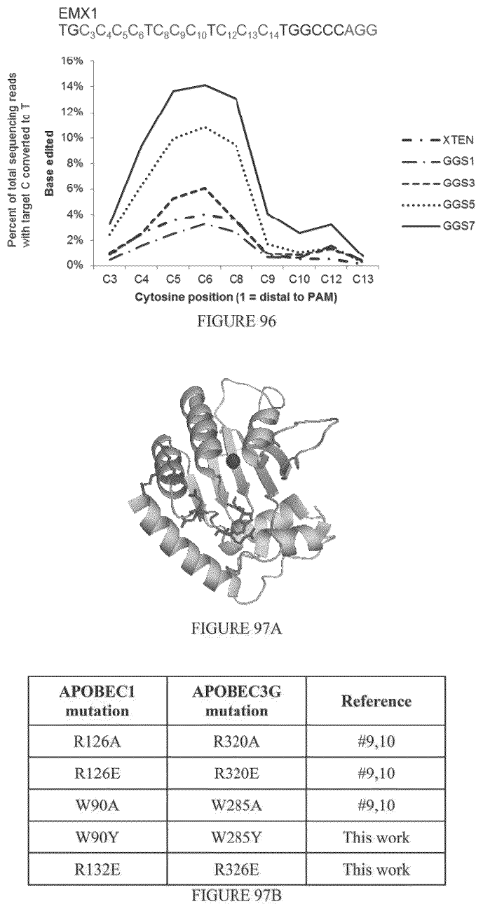

The length of the linker between APOBEC1 and dCas9 was previously observed to modulate the number of bases that are accessible by APOBEC1 in vitro¹. In HEK293T cells, however, varying the linker length did not significantly modulate the width of the editing window, suggesting that in the complex cellular milieu, the relative orientation and flexibility of dCas9 and the cytidine deaminase are not strongly determined by linker length (FIG. 96). Next, it was thought that truncating the 5′ end of the sgRNA might narrow the base editing window by reducing the length of single-stranded DNA accessible to the deaminase upon formation of the RNA-DNA heteroduplex. HEK293T cells were co-transfected with plasmids encoding BE3 and sgRNAs of different spacer lengths targeting a locus with multiple Cs in the editing window. No consistent changes in the width of base editing when using truncated sgRNAs with 17- to 19-base spacers were observed (FIGS. 95A to 95B). Truncating the sgRNA spacer to fewer than 17 bases resulted in large losses in activity (FIG. 95A).

As an alternative approach, it was thought that mutations to the deaminase domain might narrow the width of the editing window through multiple possible mechanisms. First, some mutations may alter substrate binding, the conformation of bound DNA, or substrate accessibility to the active site in ways that reduce tolerance for non-optimal presentation of a C to the deaminase active site. Second, because the high activity of APOBEC1 likely contributes to the deamination of multiple Cs per DNA binding event,^{1,13,14} mutations that reduce the catalytic efficiency of the deaminase domain of a base editor might prevent it from catalyzing successive rounds of deamination before dissociating from the DNA. Once any C:G to T:A editing event has taken place, the sgRNA no longer perfectly matches the target DNA sequence and re-binding of the base editor to the target locus should be less favorable. Both strategies were tested in an effort to discover new base editors that distinguish among multiple cytidines within the original editing window.

Given the absence of an available APOBEC1 structure, several mutations previously reported to modulate the catalytic activity of APOBEC3G, a cytidine deaminase from the same family that shares 42% sequence similarity of its active site-containing domain to that of APOBEC1, were identified¹⁵. Corresponding APOBEC1 mutations were incorporated into BE3 and evaluated their effect on base editing efficiency and editing window width in HEK293T cells at two C-rich genomic sites containing Cs at positions 3, 4, 5, 6, 8, 9, 10, 12, 13, and 14 (site A); or containing Cs at positions 5, 6, 7, 8, 9, 10, 11, and 13 (site B).

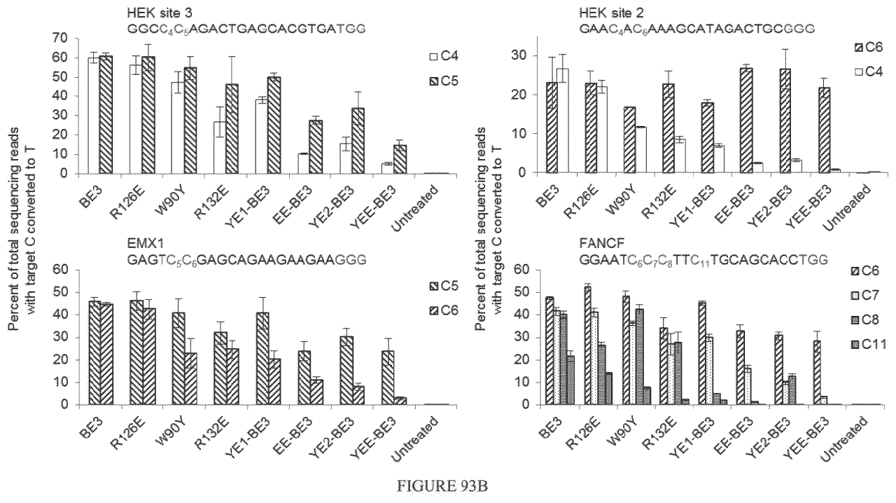

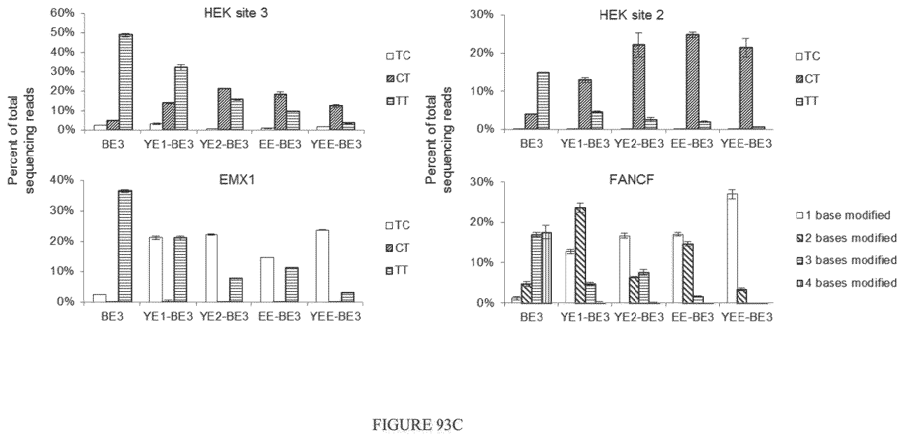

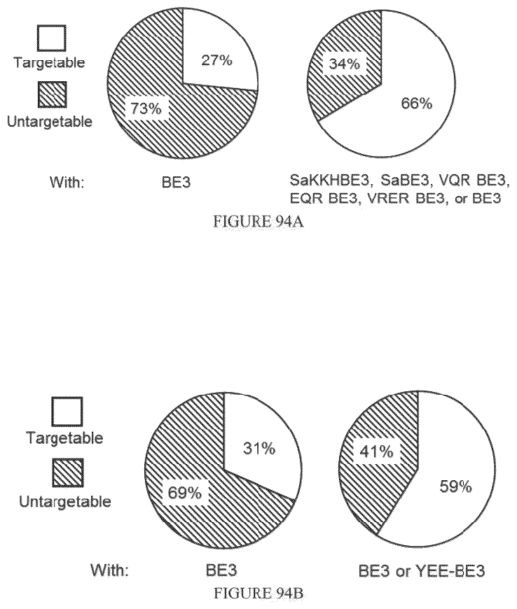

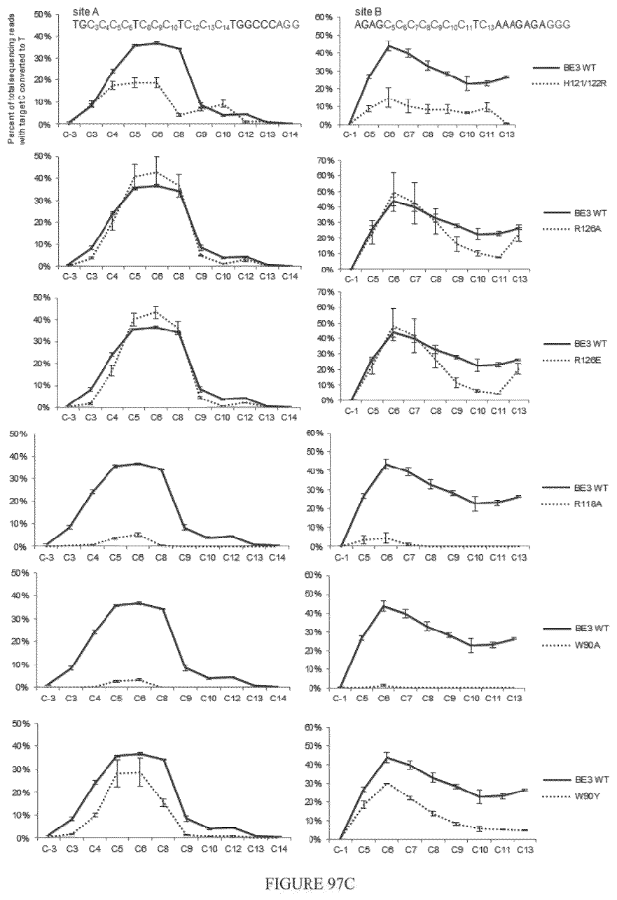

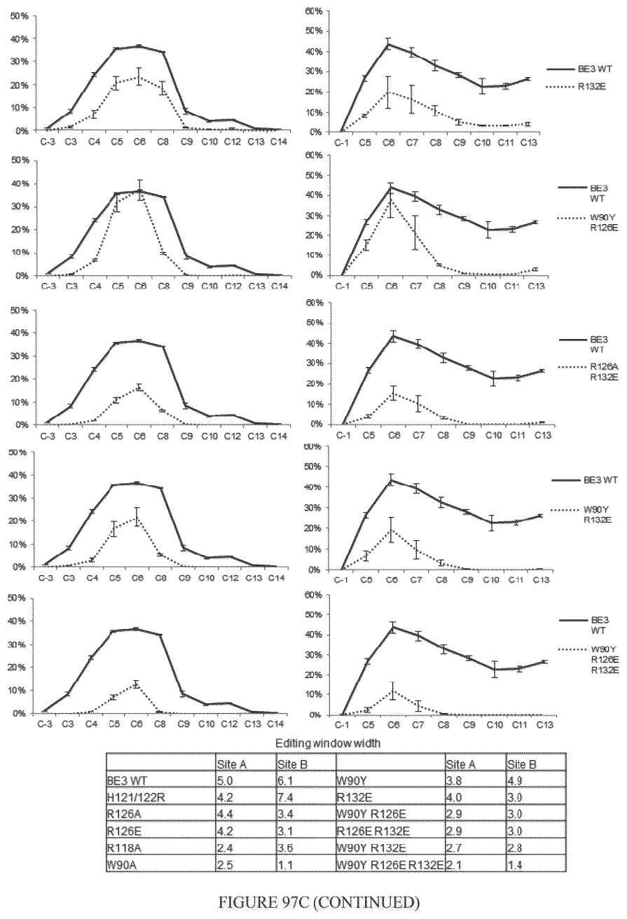

The APOBEC1 mutations R118A and W90A each led to dramatic loss of base editing efficiency (FIG. 97C). R132E led to a general decrease in editing efficiency but did not change the substantially narrow the shape of the editing window (FIG. 97C). In contrast, several mutations that narrowed the width of the editing window while maintaining substantial editing efficiency were found (FIGS. 93A and 97C). The “editing window width” was defined to represent the artificially calculated window width within which editing efficiency exceeds the half-maximal value for that target. The editing window width of BE3 for the two C-rich genomic sites tested was 5.0 (site A) and 6.1 (site B) nucleotides.

R126 in APOBEC1 is predicted to interact with the phosphate backbone of ssDNA¹³. Previous studies have shown that introducing the corresponding mutation into APOBEC3G decreased catalysis by at least 5-fold¹⁴. Interestingly, when introduced into APOBEC1 in BE3, R126A and R126E increased or maintained activity relative to BE3 at the most strongly edited positions (C5, C6, and C7), while decreasing editing activity at other positions (FIGS. 93A and 97C). Each of these two mutations therefore narrowed the width of the editing window at site A and site B to 4.4 and 3.4 nucleotides (R126A), or to 4.2 and 3.1 nucleotides (R126E), respectively (FIGS. 93A and 97C).

W90 in APOBEC1 (corresponding to W285 in APOBEC3G) is predicted to form a hydrophobic pocket in the APOBEC3G active site and assist in substrate binding¹³. Mutating this residue to Ala abrogated APOBEC3G's catalytic activity¹³. In BE3, W90A almost completely abrogated base editing efficiency (FIG. 97C). In contrast, it was found that W90Y only modestly decreased base editing activity while narrowing the editing window width at site A and site B to 3.8 and 4.9 nucleotides, respectively (FIG. 93A). These results demonstrate that mutations to the cytidine deaminase domain can narrow the activity window width of the corresponding base editors.

W90Y, R126E, and R132E, the three mutations that narrowed the editing window without drastically reducing base editing activity, were combined into doubly and triply mutated base editors. The double mutant W90Y+R126E resulted in a base editor (YE1-BE3) with BE3-like maximal editing efficiencies, but substantially narrowed editing window width (width at site A and site B=2.9 and 3.0 nucleotides, respectively (FIG. 93A). The W90Y+R132E base editor (YE2-BE3) exhibited modestly lower editing efficiencies (averaging 1.4-fold lower maximal editing yields across the five sites tested compared with BE3), and also substantially narrowed editing window width (width at site A and site B=2.7 and 2.8 nucleotides, respectively) (FIG. 97C). The R126E+R132E double mutant (EE-BE3) showed similar maximal editing efficiencies and editing window width as YE2-BE3 (FIG. 97C). The triple mutant W90Y+R126E+R132E (YEE-BE3) exhibited 2.0-fold lower average maximal editing yields but very little editing beyond the C6 position and an editing window width of 2.1 and 1.4 nucleotides for site A and site B, respectively (FIG. 97C). These data taken together indicate that mutations in the cytidine deaminase domain can strongly affect editing window widths, in some cases with minimal or only modest effects on editing efficiency.

The base editing outcomes of BE3, YE1-BE3, YE2-BE3, EE-BE3, and YEE-BE3 were further compared in HEK293T cells targeting four well-studied human genomic sites that contain multiple Cs within the BE3 activity window¹. These target loci contained target Cs at positions 4 and 5 (HEK site 3), positions 4 and 6 (HEK site 2), positions 5 and 6 (EMX1), or positions 6, 7, 8, and 11 (FANCF). BE3 exhibited little (<1.2-fold) preference for editing any Cs within the position 4-8 activity window. In contrast, YE1-BE3, exhibited a 1.3-fold preference for editing C5 over C4 (HEK site 3), 2.6-fold preference for C6 over C4 (HEK site 2), 2.0-fold preference for C5 over C6 (EMX1), and 1.5-fold preference for C6 over C7 (FANCF) (FIG. 93B). YE2-BE3 and EE-BE3 exhibited somewhat greater positional specificity (narrower activity window) than YE1-BE3, averaging 2.4-fold preference for editing C5 over C4 (HEK site 3), 9.5-fold preference for C6 over C4 (HEK site 2), 2.9-fold preference for C5 over C6 (EMX1), and 2.6-fold preference for C7 over C6 (FANCF) (FIG. 93B). YEE-BE3 showed the greatest positional selectivity, with a 2.9-fold preference for editing C5 over C4 (HEK site 3), 29.7-fold preference for C6 over C4 (HEK site 2), 7.9-fold preference for C5 over C6 (EMX1), and 7.9-fold preference for C7 over C6 (FANCF) (FIG. 93B). The findings establish that mutant base editors can discriminate between adjacent Cs, even when both nucleotides are within the BE3 editing window.

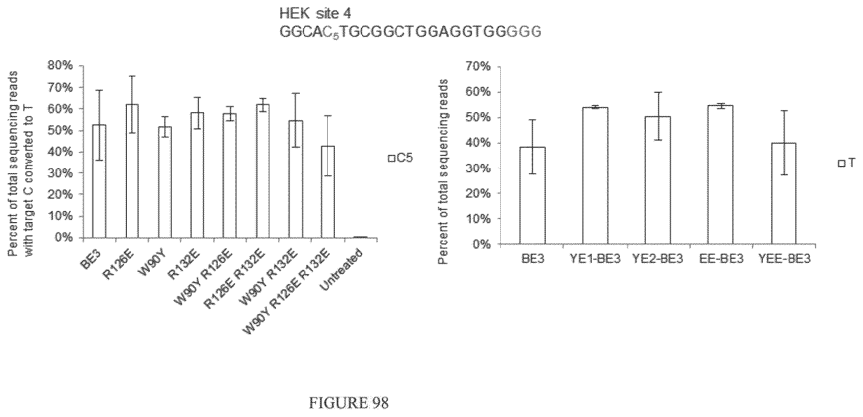

The product distributions of these four mutants and BE3 were further analyzed by HTS to evaluate their apparent processivity. BE3 generated predominantly T4-T5 (HEK site 3), T4-T6 (HEK site 2), and T5-T6 (EMX1) products in treated HEK293T cells, resulting in, on average, 7.4-fold more products containing two Ts, than products containing a single T. In contrast, YE1-BE3, YE2-BE3, EE-BE3, and YEE-BE3 showed substantially higher preferences for singly edited C4-T5, C4-T6, and T5-C6 products (FIG. 93C). YE1-BE3 yielded products with an average single-T to double-T product ratio of 1.4. YE2-BE3 and EE-BE3 yielded products with an average single-T to double-T product ratio of 4.3 and 5.1, respectively (FIG. 93C). Consistent with the above results, the YEE-BE3 triple mutant favored single-T products by an average of 14.3-fold across the three genomic loci. (FIG. 93C). For the target site in which only one C is within the target window (HEK site 4, at position C5), all four mutants exhibited comparable editing efficiencies as BE3 (FIG. 98). These findings indicate that these BE3 mutants have decreased apparent processivity and can favor the conversion of only a single C at target sites containing multiple Cs within the BE3 editing window. These data also suggest a positional preference of C5>C6>C7≈C4 for these mutant base editors, although this preference could differ depending on the target sequence.

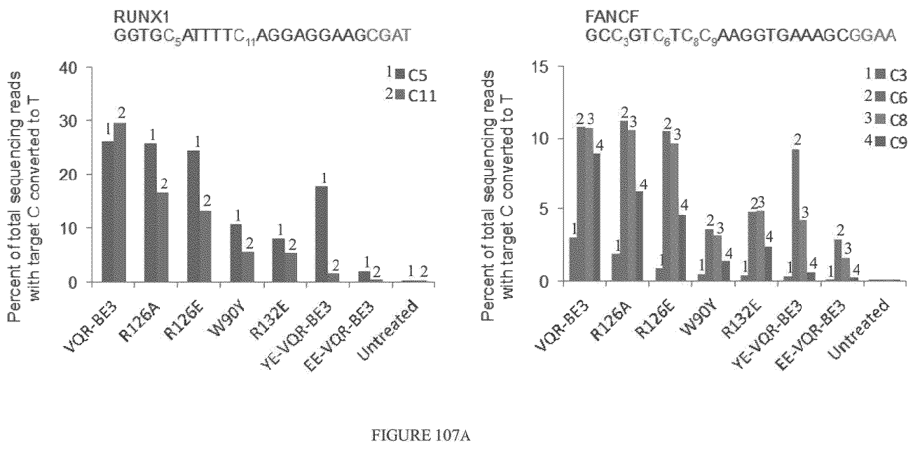

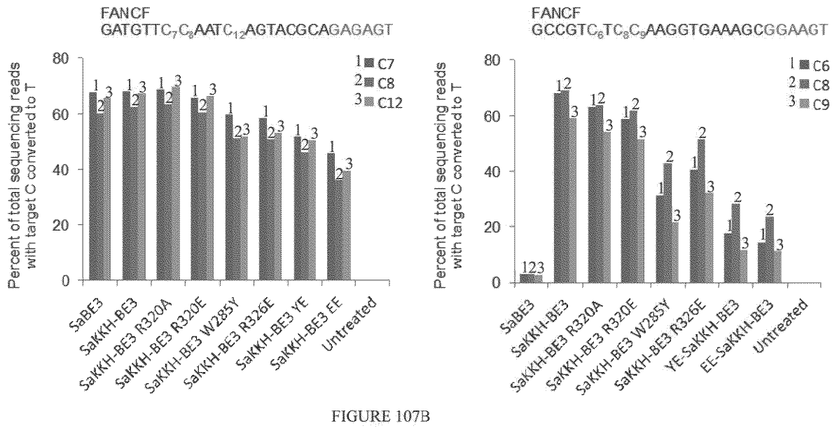

The window-modulating mutations in APOBEC1 were applied to VQR-BE3, allowing selective base editing of substrates at sites targeted by NGA PAM (FIG. 107A). However, when these mutations were applied to SaKKH-BE3, a linear decrease in base editing efficiency was observed without the improvement in substrate selectivity, suggesting a different kinetic equilibrium and substrate accessibility of this base editor than those of BE3 and its variants (FIG. 107B).

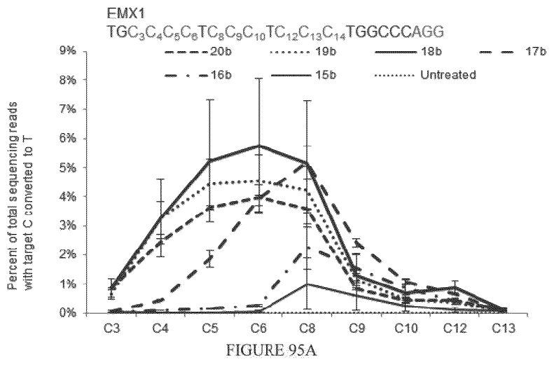

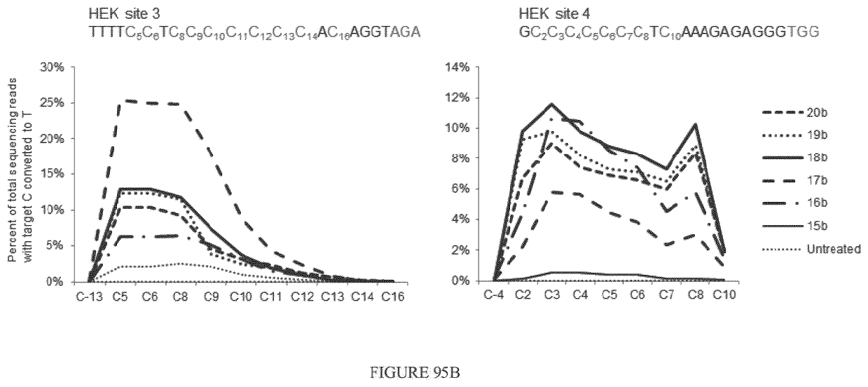

The five base editors with altered PAM specificities described in this study together increase the number of disease-associated mutations in the ClinVar database that can in principle be corrected by base editing by 2.5-fold (FIGS. 94A and 94B). Similarly, the development of base editors with narrowed editing windows approximately doubles the fraction of ClinVar entries with a properly positioned NGG PAM that can be corrected by base editing without comparable modification of a non-target C (from 31% for BE3 to 59% for YEE-BE3) (FIGS. 94A and 94B).

In summary, the targeting scope of base editing was substantially expanded by developing base editors that use Cas9 variants with different PAM specificities, and by developing a collection of deaminase mutants with varying editing window widths. In theory, base editing should be possible using other programmable DNA-binding proteins (such as Cpf1¹⁶) that create a bubble of single-stranded DNA that can serve as a substrate for a single-strand-specific nucleotide deaminase enzyme.

### Materials and Methods

Cloning. PCR was performed using Q5 Hot Start High-Fidelity DNA Polymerase (New England Biolabs). Plasmids for BE and sgRNA were constructed using USER cloning (New England Biolabs), obtained from previously reported plasmids¹. DNA vector amplification was carried out using NEB 10beta competent cells (New England Biolabs).

Cell culture. HEK293T (ATCC CRL-3216) were cultured in Dulbecco's Modified Eagle's Medium plus GlutaMax (ThermoFisher) supplemented with 10% (v/v) fetal bovine serum (FBS), at 37° C. with 5% CO₂. Immortalized rat astrocytes containing the ApoE4 isoform of the APOE gene (Taconic Biosciences) were maintained in Dulbecco's Modified Eagle's Medium plus GlutaMax (ThermoFisher Scientific) supplemented with 10% (v/v) fetal bovine serum (FBS) and 200 μg/mL Geneticin (ThermoFisher Scientific).

Transfections. HEK293T cells were seeded on 48-well collagen-coated BioCoat plates (Corning) and transfected at approximately 85% confluency. 750 ng of BE and 250 ng of sgRNA expression plasmids were transfected using 1.5 μl of Lipofectamine 2000 (ThermoFisher Scientific) per well according to the manufacturer's protocol.

High-throughput DNA sequencing of genomic DNA samples. Transfected cells were harvested after 3 d and the genomic DNA was isolated using the Agencourt DNAdvance Genomic DNA Isolation Kit (Beckman Coulter) according to the manufacturer's instructions. Genomic regions of interest were amplified by PCR with flanking HTS primer pairs listed in the Supplementary Sequences. PCR amplification was carried out with Phusion hot-start II DNA polymerase (ThermoFisher) according to the manufacturer's instructions. PCR products were purified using RapidTips (Diffinity Genomics). Secondary PCR was performed to attach sequencing adaptors. The products were gel-purified and quantified using the KAPA Library Quantification Kit-Illumina (KAPA Biosystems). Samples were sequenced on an Illumina MiSeq as previously described¹.

Data analysis. Nucleotide frequencies were assessed using a previously described MATLAB script 1. Briefly, the reads were aligned to the reference sequence via the Smith-Waterman algorithm. Base calls with Q-scores below 30 were replaced with a placeholder nucleotide (N). This quality threshold results in nucleotide frequencies with an expected theoretical error rate of 1 in 1000.

Analyses of base editing processivity were performed using a custom python script. This program trims sequencing reads to the 20 nucleotide protospacer sequence as determined by a perfect match for the 7 nucleotide sequences that should flank the target site. These targets were then consolidated and sorted by abundance to assess the frequency of base editing products.

Bioinformatic analysis of the ClinVar database of human disease-associated mutations was performed in a manner similar to that previously described but with small adjustments¹. These adjustments enable the identification of targets with PAMs of customizable length and sequence. In addition, this improved script includes a priority ranking of target C positions (C5>C6>C7>C8≈C4), thus enabling the identification of target sites in which the on-target C is either the only cytosine within the window or is placed at a position with higher predicted editing efficiency than any off-target C within the editing window.

### REFERENCES FOR EXAMPLE 12

1 Komor, A. C. et al. Programmable editing of a target base in genomic DNA without double-stranded DNA cleavage. Nature 533, 420-424 (2016).
2 Sander, J. D. & Joung, J. K. CRISPR-Cas systems for editing, regulating and targeting genomes. Nature biotechnology 32, 347-355 (2014).
3 Cong, L. et al. Multiplex genome engineering using CRISPR/Cas systems. Science 339, 819-823 (2013).
4 Ran, F. A. et al. Genome engineering using the CRISPR-Cas9 system. Nat. Protocols 8, 2281-2308 (2013).
5 Landrum, M. J. et al. ClinVar: public archive of interpretations of clinically relevant variants. Nucleic Acids Res. 44, D862-D868 (2015).
6 Nishida, K. et al. Targeted nucleotide editing using hybrid prokaryotic and vertebrate adaptive immune systems. Science 353, aaf8729-1-8 (2016).
7 Ma, Y. et al. Targeted AID-mediated mutagenesis (TAM) enables efficient genomic diversification in mammalian cells. Nat. Methods doi:10.1038/nmeth.4027 (2016).
8 Jiang, F. et al. Structures of a CRISPR-Cas9 R-loop complex primed for DNA cleavage. Science 351, 867-71 (2016).
9 Ran, F. A. et al. In vivo genome editing using Staphylococcus aureus Cas9. Nature 520, 186-191 (2015).
10 Zhang, Y. et al. Comparison of non-canonical PAMs for CRISPR/Cas9-mediated DNA cleavage in human cells. Sci. Rep. 4, (2014).
11 Kleinstiver, B. P. et. al. Engineered CRISPR-Cas9 nucleases with altered PAM specificities. Nature 523, 481-485 (2015).
12 Kleinstiver, B. P. et. al. Broadening the targeting range of Staphylococcus aureus CRISPR-Cas9 by modifying PAM recognition. Nat. Biotechnol. 33, 1293-1298 (2015).
13 Holden, L. G. et al. Crystal structure of the anti-viral APOBEC3G catalytic domain and functional implications. Nature 452, 121-124 (2008).
14 Chen, K.-M. et al. Structure of the DNA deaminase domain of the HIV-1 restriction factor APOBEC3G. Nature 452, 116-119 (2008).
15 Harris, R. S., Petersen-Mahrt, S. K. & Neuberger, M. S. RNA Editing Enzyme APOBEC1 and Some of Its Homologs Can Act as DNA Mutators. Molecular Cell 10, 1247-1253 (2002).
16 Zetsche, B. et al. Cpf1 Is a Single RNA-Guided Endonuclease of a Class 2 CRISPR-Cas System. Cell 163, 759-771 (2015).

### Example 13

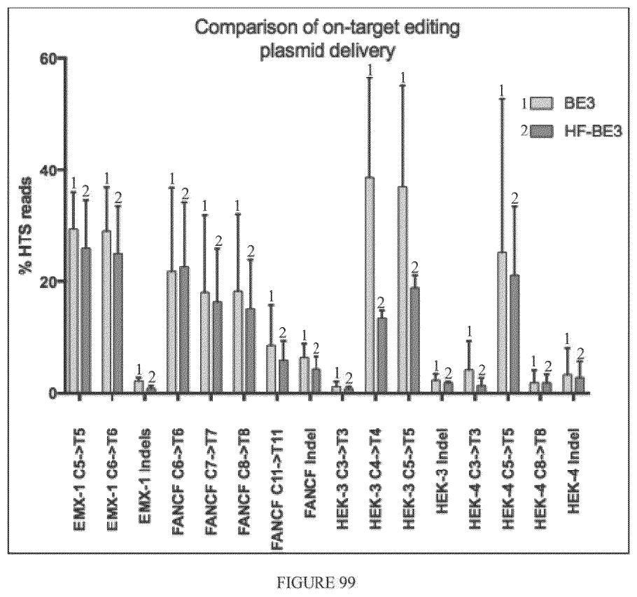

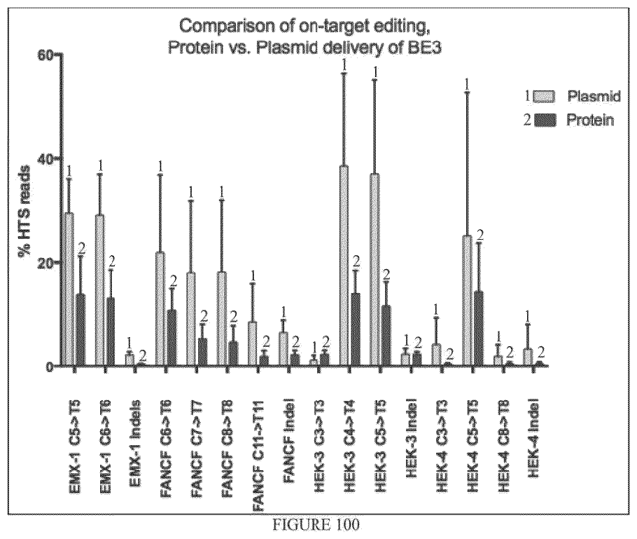

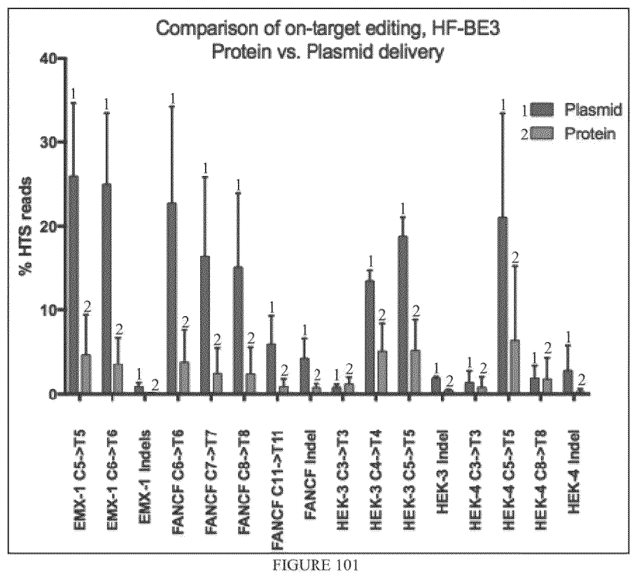

Using improved transfection procedures and better plasmids, biological replicates (n=3) were used to install the four HF mutations into the Cas9 portion of BE3. The mutations do not significantly effect on-targeting editing with plasmid delivery (FIG. 99). At the tested concentration, BE3 protein delivery works; however, the on-target editing is lower than for plasmid delivery (FIG. 100). Protein delivery of BE3 with the HF mutations installed reduces on-targeting editing efficiency but still yields some edited cells (FIG. 101).

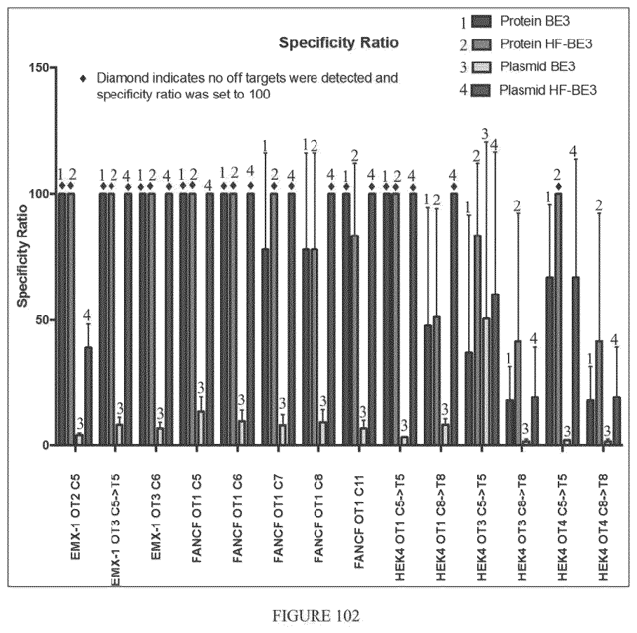

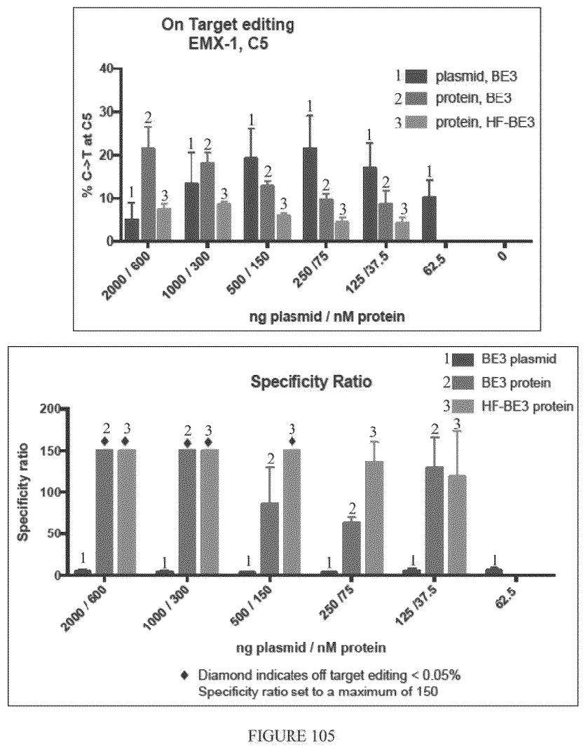

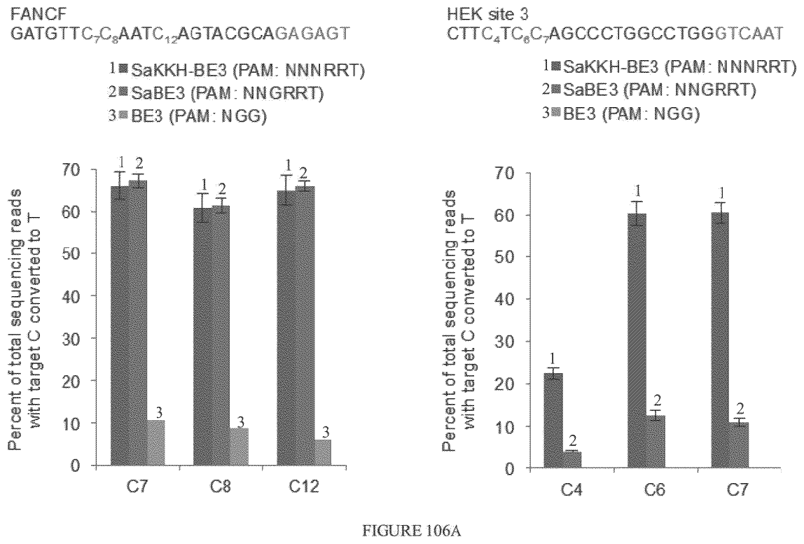

Both lipofection and installing HF mutations were shown to decrease off-target deamination events. For the four sites shown in FIG. 102, the off-target sitest (OT) with the highest GUIDE-Seq reads and deamination events were assayed (Komor et al., Nature, 2016). The specificity ratio was calculated by dividing the off-target editing by the on-target editing at the closest corresponding C. In cases where off-target editing was not detectable, the ratio was set to 100. Thus, a higher specificity ratio indicates a more specific construct. BE3 plasmid delivery showed much higher off-target/on-target editing than protein delivery of BE3, plasmid delivery of HF-BE3, or protein delivery of HF-BE3 (FIGS. 102 and 105).

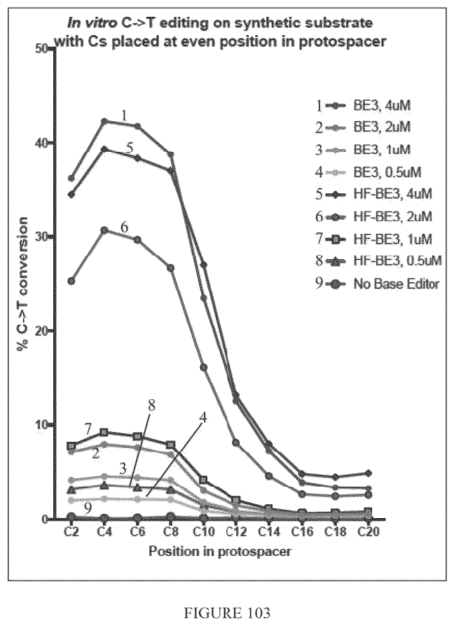

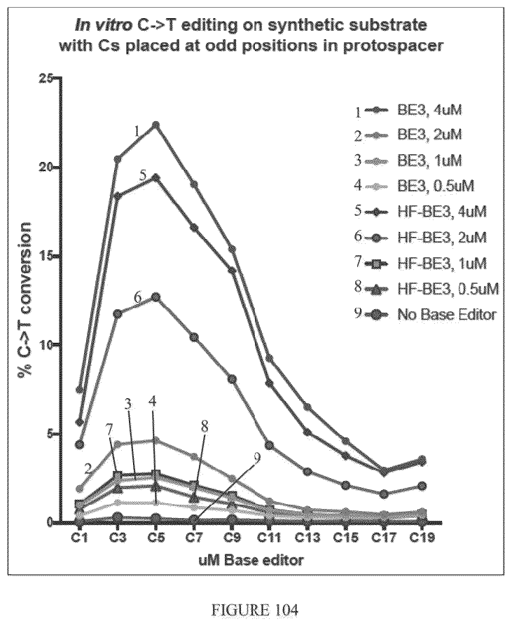

Purified proteins HF-BE3 and BE3 were analyzed in vitro for their capabilities to convert C to T residues at different positions in the spacer with the most permissive motif. Both BE3 and HF-BE3 proteins were found to have the same “window” for base editing (FIGS. 103 and 104).

A list of the disease targets is given in Table 9. The base to be edited in Table 9 is indicated in bold and underlined.

TABLE 9
Base Editor Disease Targets

| GENE | DISEASE | SPACER | PAM | EDITOR | DEFECT | CELL |
|---|---|---|---|---|---|---|
| RB1 | RETINOBLA | AATCTAGTAAA | AAAA | SAKKH- | SPLICING | J82 |
| | STOMA | TAAATTGATGT | GT | BE3 | IMPAIR- | |
| | | | | | MENT | |
| PTEN | CANCER | GACCAACGGCT | TGA | VQR- | W111R | MC11 |
| | | AAGTGAAGA | | BE3 | | 6 |
| PIK3C | CANCER | TCCTTTCTTCA | ACTG | SAKKH- | K111R | CRL- |
| A | | CGGTTGCCT | GT | BE3 | | 5853 |
| PIK3C | CANCER | CTCCTGCTCAG | AGA | VQR- | Q546R | CRL- |
| A | | TGATTTCAG | | BE3 | | 2505 |
| TP53 | CANCER | TGTCACACATG | TGG | YEE- | N239D | SNU47 |
| | | TAGTTGTAG | | BE3 | | 5 |
| HRAS | CANCER | CCTCCCGGCCG | AGG | YEE- | Q61R | MC/C |
| | | GCGGTATCC | | BE3 | | AR |

TABLE 6
Exemplary diseases that may be treated using
base editors. The protospacer and PAM
sequences are shown in the sgRNA (PAM) column.
The PAM sequence is shown in parentheses and
with the base to be edited indicated by
underlining.

| Disease | gene | Base | sgRNA | Base |
|---|---|---|---|---|
| target | symbol | changed | (PAM) | editor |
| Prion | PRNP | R37* | GGCAGCCGAT | BE3 |
| disease | | | ACCCGGGGCA | |
| | | | (GGG) | |
| | | | GGGCAGCCGA | |
| | | | TACCCGGGGC | |
| | | | (AGG) | |
| Pendred | Slc26a4 | c.919-2A>G | TTATTGTCCG | BE3 |
| syndrome | | | AAATAAAAGA | |
| | | | (AGA) | |
| | | | ATTGTCCGAA | (VQR |
| | | | ATAAAAGAAG | SaCas9) |
| | | | (AGG) | |
| | | | TTGTCCGAAA | |
| | | | TAAAAGAAGA | |
| | | | (GGA) | |
| | | | GTCCGAAATA | |
| | | | AAAGAAGAGG | |
| | | | AAAA(AAT) | |
| | | | GTCCGAAATA | |
| | | | AAAGAAGAGG | |
| | | | AAAAA(ATT) | |
| Congenital | Tmc1 | c.545A>G | CAGGAAGCAC | BE3 |
| deafness | | | GAGGCCACTG | |
| | | | (AGG) | |
| | | | AACAGGAAGC | YE-BE3 |
| | | | ACGAGGCCAC | |
| | | | (TGA) | |
| | | | AGGAAGCACG | YEE-BE3 |
| | | | AGGCCACTGA | |
| | | | (GGA) | |
| Acquired | SNHL | S33F | TTGGATTCTG | BE3 |
| deafness | | | GAATCCATTC | |
| | | | (TGG) | |
| Alzheimer's | APP | A673T | TCTGCATCCA | BE3 VQR |
| Disease | | | TCTTCACTTC | |
| | | | (AGA) | |
| Niemann- | NPC1 | I1061T | CTTACAGCCA | BE3 VQR |
| Pick | | | GTAATGTCAC | |
| Disease | | | (CGA) | |
| Type C | | | | |

Additional exemplary genes in the human genome that may be targeted by the base editors or complexes of this disclosure are provided herein in Tables 7 and 8. Table 7 includes gene mutations that may be corrected by changing a cytosine (C) to a thymine (T), for example, using a BE3 nucleobase editor. Table 8 includes gene mutations that may be corrected by changing a guanine (G) to an adenine (A), for example, using a BE3 nucleobase

Lengthy table referenced here
US20250270527A1-20250828-T00002
Please refer to the end of the specification for access instructions.

### REFERENCES

1. Humbert O, Davis L, Maizels N. Targeted gene therapies: tools, applications, optimization. Crit Rev Biochem Mol. 2012; 47(3):264-81. PMID: 22530743.
2. Perez-Pinera P, Ousterout D G, Gersbach C A. Advances in targeted genome editing. Curr Opin Chem Biol. 2012; 16(3-4):268-77. PMID: 22819644.
3. Urnov F D, Rebar E J, Holmes M C, Zhang H S, Gregory P D. Genome editing with engineered zinc finger nucleases. Nat Rev Genet. 2010; 11(9):636-46. PMID: 20717154.
4. Joung J K, Sander J D. TALENs: a widely applicable technology for targeted genome editing. Nat Rev Mol Cell Biol. 2013; 14(1):49-55. PMID: 23169466.
5. Charpentier E, Doudna J A. Biotechnology: Rewriting a genome. Nature. 2013; 495, (7439):50-1. PMID: 23467164.
6. Pan Y, Xia L, Li A S, Zhang X, Sirois P, Zhang J, Li K. Biological and biomedical applications of engineered nucleases. Mol Biotechnol. 2013; 55(1):54-62. PMID: 23089945.
7. De Souza, N. Primer: genome editing with engineered nucleases. Nat Methods. 2012; 9(1):27. PMID: 22312638.
8. Santiago Y, Chan E, Liu P Q, Orlando S, Zhang L, Urnov F D, Holmes M C, Guschin D, Waite A, Miller J C, Rebar E J, Gregory P D, Klug A, Collingwood T N. Targeted gene knockout in mammalian cells by using engineered zinc-finger nucleases. Proc Natl Acad Sci USA. 2008; 105(15):5809-14. PMID: 18359850.
9. Cargill M, Altshuler D, Ireland J, Sklar P, Ardlie K, Patil N, Lane C R, Lim E P, Kalyanaraman N, Nemesh J, Ziaugra L, Friedland L, Rolfe A, Warrington J, Lipshutz R, Daley G Q, Lander E S. Characterization of single-nucleotide polymorphisms in coding regions of human genes. Nat Genet. 1999; 22(3):231-8. PMID: 10391209.
10. Jansen R, van Embden J D, Gaastra W, Schouls L M. Identification of genes that are associated with DNA repeats in prokaryotes. Mol Microbiol. 2002; 43(6):1565-75. PMID: 11952905.
11. Mali P, Esvelt K M, Church G M. Cas9 as a versatile tool for engineering biology. Nat Methods. 2013; 10(10):957-63. PMID: 24076990.
12. Jore M M, Lundgren M, van Duijin E, Bultema J B, Westra E R, Waghmare S P, Wiedenheft B, Pul U, Wurm R, Wagner R, Beijer M R, Barendregt A, Shou K, Snijders A P, Dickman M J, Doudna J A, Boekema E J, Heck A J, van der Oost J, Brouns S J. Structural basis for CRISPR RNA-guided DNA recognition by Cascade. Nat Struct Mol Biol. 2011; 18(5):529-36. PMID: 21460843.
13. Horvath P, Barrangou R. CRISPR/Cas, the immune system of bacteria and archaea. Science. 2010; 327(5962):167-70. PMID: 20056882.
14. Wiedenheft B, Sternberg S H, Doudna J A. RNA-guided genetic silencing systems in bacteria and archaea. Nature. 2012; 482(7385):331-8. PMID: 22337052.
15. Gasiunas G, Siksnys V. RNA-dependent DNA endonuclease Cas9 of the CRISPR system: Holy Grail of genome editing?Trends Microbiol. 2013; 21(11):562-7. PMID: 24095303.
16. Qi L S, Larson M H, Gilbert L A, Doudna J A, Weissman J S, Arkin A P, Lim W A. Repurposing CRISPR as an RNA-guided platform for sequence-specific control of gene expression. Cell. 2013; 152(5):1173-83. PMID: 23452860.
17. Perez-Pinera P, Kocak D D, Vockley C M, Adler A F, Kabadi A M, Polstein L R, Thakore P I, Glass K A, Ousterout D G, Leong K W, Guilak F, Crawford G E, Reddy T E, Gersbach C A. RNA-guided gene activation by CRISPR-Cas9-based transcription factors. Nat Methods. 2013; 10(10):973-6. PMID: 23892895.
18. Mali P, Aach J, Stranges P B, Esvelt K M, Moosburner M, Kosuri S, Yang L, Church G M. CAS9 transcriptional activators for target specificity screening and paired nickases for cooperative genome engineering. Nat Biotechnol. 2013; 31(9):833-8. PMID: 23907171.
19. Gilbert L A, Larson M H, Morsut L, Liu Z, Brar G A, Torres S E, Stern-Ginossar N, Brandman O, Whitehead E H, Doudna J A, Lim W A, Weissman J S, Qi L S. CRISPR-mediated modular RNA-guided regulation of transcription in eukaryotes. Cell. 2013; 154(2):442-51. PMID: 23849981.
20. Larson M H, Gilbert L A, Wang X, Lim W A, Weissman J S, Qi L S. CRISPR interference (CRISPRi) for sequence-specific control of gene expression. Nat Protoc. 2013; 8(11):2180-96. PMID: 24136345.
21. Mali P, Yang L, Esvelt K M, Aach J, Guell M, DiCarlo J E, Norville J E, Church G M. RNA-guided human genome engineering via Cas9. Science. 2013; 339(6121):823-6. PMID: 23287722.
22. Cole-Strauss A, Yoon K, Xiang Y, Byrne B C, Rice M C, Gryn J, Holloman W K, Kmiec E B. Correction of the mutation responsible for sickle cell anemia by an RNA-DNA oligonucleotide. Science. 1996; 273(5280):1386-9. PMID: 8703073.
23. Tagalakis A D, Owen J S, Simons J P. Lack of RNA-DNA oligonucleotide (chimeraplast) mutagenic activity in mouse embryos. Mol Reprod Dev. 2005; 71(2):140-4. PMID: 15791601.
24. Ray A, Langer M. Homologous recombination: ends as the means. Trends Plant Sci. 2002; 7(10):435-40. PMID 12399177.
25. Britt A B, May G D. Re-engineering plant gene targeting. Trends Plant Sci. 2003; 8(2):90-5. PMID: 12597876.
26. Vagner V, Ehrlich S D. Efficiency of homologous DNA recombination varies along the Bacillus subtilis chromosome. J Bacteriol. 1988; 170(9):3978-82. PMID: 3137211.
27. Saleh-Gohari N, Helleday T. Conservative homologous recombination preferentially repairs DNA double-strand breaks in the S phase of the cell cycle in human cells. Nucleic Acids Res. 2004; 32(12):3683-8. PMID: 15252152.
28. Lombardo A, Genovese P, Beausejour C M, Colleoni S, Lee Y L, Kim K A, Ando D, Urnov F D, Galli C, Gregory P D, Holmes M C, Naldini L. Gene editing in human stem cells using zinc finger nucleases and integrase-defective lentiviral vector delivery. Nat Biotechnol. 2007; 25(11):1298-306. PMID: 17965707.
29. Conticello S G. The AID/APOBEC family of nucleic acid mutators. Genome Biol. 2008; 9(6):229. PMID: 18598372.
30. Reynaud C A, Aoufouchi S, Faili A, Weill J C. What role for AID: mutator, or assembler of the immunoglobulin mutasome?Nat Immunol. 2003; 4(7):631-8.
31. Bhagwat A S. DNA-cytosine deaminases: from antibody maturation to antiviral defense. DNA Repair (Amst). 2004; 3(1):85-9. PMID: 14697763.
32. Navaratnam N, Sarwar R. An overview of cytidine deaminases. Int J Hematol. 2006; 83(3):195-200. PMID: 16720547.
33. Holden L G, Prochnow C, Chang Y P, Bransteitter R, Chelico L, Sen U, Stevens R C, Goodman M F, Chen X S. Crystal structure of the anti-viral APOBEC3G catalytic domain and functional implications. Nature. 2008; 456(7218):121-4. PMID: 18849968.
34. Chelico L, Pham P, Petruska J, Goodman M F. Biochemical basis of immunological and retroviral responses to DNA-targeted cytosine deamination by activation-induced cytidine deaminase and APOBEC3G. J Biol Chem. 2009; 284(41). 27761-5. PMID: 19684020.
35. Pham P, Bransteitter R, Goodman M F. Reward versus risk: DNA cytidine deaminases triggering immunity and disease. Biochemistry. 2005; 44(8):2703-15. PMID 15723516.
36. Chen X, Zaro J L, Shen W C. Fusion protein linkers: property, design and functionality. Adv Drug Deliv Rev. 2013; 65(10):1357-69. PMID: 23026637.
37. Lee J W, Soung Y H, Kim S Y, Lee H W, Park W S, Nam S W, Kim S H, Lee J Y, Yoo N J, Lee S H. PIK3CA gene is frequently mutated in breast carcinomas and hepatocellular carcinomas. Oncogene. 2005; 24(8):1477-80. PMID: 15608678.
38. Ikediobi O N, Davies H, Bignell G, Edkins S, Stevens C, O'Meara S, Santarius T, Avis T, Barthorpe S, Brackenbury L, Buck G, Butler A, Clements J, Cole J, Dicks E, Forbes S, Gray K, Halliday K, Harrison R, Hills K, Hinton J, Hunter C, Jenkinson A, Jones D, Kosmidou V, Lugg R, Menzies A, Mironenko T, Parker A, Perry J, Raine K, Richardson D, Shepherd R, Small A, Smith R, Solomon H, Stephens P, Teaque J, Tofts C, Varian J, Webb T, West S, Widaa S, Yates A, Reinhold W, Weinstein J N, Stratton M R, Futreal P A, Wooster R. Mutation analysis of 24 known cancer genes in the NCI-60 cell line set. Mol Cancer Ther. 2006; 5(11):2606-12. PMID: 17088437.
39. Cox, D. B., Platt, R. J. & Zhang, F. Therapeutic genome editing: prospects and challenges. Nature medicine 21, 121-131, doi:10.1038/nm.3793 (2015).
40. Hilton, I. B. & Gersbach, C. A. Enabling functional genomics with genome engineering. Genome research 25, 1442-1455, doi:10.1101/gr.190124.115 (2015).
41. Sander, J. D. & Joung, J. K. CRISPR-Cas systems for editing, regulating and targeting genomes. Nature biotechnology 32, 347-355, doi:10.1038/nbt.2842 (2014).
42. Maruyama, T. et al. Increasing the efficiency of precise genome editing with CRISPR-Cas9 by inhibition of nonhomologous end joining. Nature biotechnology 33, 538-542, doi:10.1038/nbt.3190 (2015).
43. Chu, V. T. et al. Increasing the efficiency of homology-directed repair for CRISPR-Cas9-induced precise gene editing in mammalian cells. Nature biotechnology 33, 543-548, doi:10.1038/nbt.3198 (2015).
44. Lin, S., Staahl, B. T., Alla, R. K. & Doudna, J. A. Enhanced homology-directed human genome engineering by controlled timing of CRISPR/Cas9 delivery. eLife 3, e04766, doi:10.7554/eLife.04766 (2014).
45. Cong, L. et al. Multiplex genome engineering using CRISPR/Cas systems. Science 339, 819-823, doi:10.1126/science.1231143 (2013).
46. Rong, Z., Zhu, S., Xu, Y. & Fu, X. Homologous recombination in human embryonic stem cells using CRISPR/Cas9 nickase and a long DNA donor template. Protein & cell 5, 258-260, doi:10.1007/s13238-014-0032-5 (2014).
47. Jinek, M. et al. A Programmable Dual-RNA-Guided DNA Endonuclease in Adaptive Bacterial Immunity. Science 337, 816-821, doi:10.1126/science.1225829 (2012).
48. Harris, R. S., Petersen-Mahrt, S. K. & Neuberger, M. S. RNA Editing Enzyme APOBEC1 and Some of Its Homologs Can Act as DNA Mutators. Molecular Cell 10, 1247-1253 (2002).
49. Jinek, M. et al. Structures of Cas9 endonucleases reveal RNA-mediated conformational activation. Science 343, 1247997, doi:10.1126/science.1247997 (2014).
50. Schellenberger, V. et al. A recombinant polypeptide extends the in vivo half-life of peptides and proteins in a tunable manner. Nature biotechnology 27, 1186-1190, doi:10.1038/nbt.1588 (2009).
51. Saraconi, G., Severi, F., Sala, C., Mattiuz, G. & Conticello, S. G. The RNA editing enzyme APOBEC1 induces somatic mutations and a compatible mutational signature is present in esophageal adenocarcinomas. Genome biology 15,417-(2014).
52. Anders, C., Niewoehner, O., Duerst, A. & Jinek, M. Structural basis of PAM-dependent target DNA recognition by the Cas9 endonuclease. Nature 513, 569-573, doi:10.1038/nature13579 (2014).
53. Cong, L. et al. Multiplex genome engineering using CRISPR/Cas systems. Science 339, 819-823 (2013).
54. Tsai, S. Q. et al. GUIDE-seq enables genome-wide profiling of off-target cleavage by CRISPR-Cas nucleases. Nature biotechnology 33, 187-197, doi:10.1038/nbt.3117 (2015).
55. Kunz, C., Saito, Y. & Schar, P. DNA Repair in mammalian cells: Mismatched repair: variations on a theme. Cellular and molecular life sciences: CMLS 66, 1021-1038, doi:10.1007/s00018-009-8739-9 (2009).
56. D., M. C. et al. Crystal structure of human uracil-DNA glycosylase in complex with a protein inhibitor: protein mimicry of DNA. Cell 82, 701-708 (1995).
57. Caldecott, K. W. Single-strand break repair and genetic disease. Nature reviews. Genetics 9, 619-631, doi:10.1038/nrg2380 (2008).
58. Fukui, K. DNA mismatch repair in eukaryotes and bacteria. Journal of nucleic acids 2010, doi:10.4061/2010/260512 (2010).
59. Gasiunas, G., Barrangou, R., Horvath, P. & Siksnys, V. Cas9-crRNA ribonucleoprotein complex mediates specific DNA cleavage for adaptive immunity in bacteria. Proceedings of the National Academy of Sciences 109, E2579-E2586, doi:10.1073/pnas.1208507109 (2012).
60. Ran, F. A. et al. In vivo genome editing using Staphylococcus aureus Cas9. Nature 520, 186-191, doi:10.1038/nature14299 (2015).
61. Kuscu, C., Arslan, S., Singh, R., Thorpe, J. & Adli, M. Genome-wide analysis reveals characteristics of off-target sites bound by the Cas9 endonuclease. Nature biotechnology 32, 677-683, doi:10.1038/nbt.2916(2014).
62. Wu, X. et al. Genome-wide binding of the CRISPR endonuclease Cas9 in mammalian cells. Nature biotechnology 32, 670-676, doi:10.1038/nbt.2889 (2014).
63. Beale, R. C. L. et al. Comparison of the Differential Context-dependence of DNA Deamination by APOBEC Enzymes: Correlation with Mutation Spectra in Vivo. Journal of Molecular Biology 337, 585-596, doi:10.1016/j.jmb.2004.01.046 (2004).
64. Kim, J., Basak, J. M. & Holtzman, D. M. The role of apolipoprotein E in Alzheimer's disease. Neuron 63, 287-303, doi:10.1016/j.neuron.2009.06.026 (2009).
65. Liu, C. C., Kanekiyo, T., Xu, H. & Bu, G. Apolipoprotein E and Alzheimer disease: risk, mechanisms and therapy. Nature reviews. Neurology 9, 106-118, doi:10.1038/nrneurol.2012.263 (2013).
66. Sjöblom, T. et al. The Consensus Coding Sequences of Human Breast and Colorectal Cancers. Science 314, 268-274, doi:10.1126/science.1133427 (2006).
67. Stephens, P. J. et al. The landscape of cancer genes and mutational processes in breast cancer. Nature 486, 400-404, doi:10.1038/nature11017 (2012).
68. Landrum, M. J. et al. ClinVar: public archive of interpretations of clinically relevant variants. Nucleic Acids Research, doi:10.1093/nar/gkv1222 (2015).
69. Slaymaker, I. M. et al. Rationally engineered Cas9 nucleases with improved specificity. Science, doi:10.1126/science.aad5227 (2015).
70. Davis, K. M., Pattanayak, V., Thompson, D. B., Zuris, J. A. & Liu, D. R. Small molecule-triggered Cas9 protein with improved genome-editing specificity. Nature chemical biology 11, 316-318, doi:10.1038/nchembio.1793 (2015).
71. Zuris, J. A. et al. Cationic lipid-mediated delivery of proteins enables efficient protein-based genome editing in vitro and in vivo. Nature biotechnology 33, 73-80, doi:10.1038/nbt.3081 (2015).
72. Kleinstiver, B. P. et al. Engineered CRISPR-Cas9 nucleases with altered PAM specificities. Nature 523, 481-485, doi:10.1038/naturel4592 (2015).
73. Pattanayak, V. et al. High-throughput profiling of off-target DNA cleavage reveals RNA-programmed Cas9 nuclease specificity. Nature Biotechnology 31, 839-843, doi:10.1038/nbt.2673 (2013).
74. Shcherbakova, D. M. & Verkhusha, V. V. Near-infrared fluorescent proteins for multicolor in vivo imaging. Nature Methods 10, 751-754, doi:10.1038/nmeth.2521 (2013).
75. Ran, F. A. et al. Genome engineering using the CRISPR-Cas9 system. Nat. Protocols 8, 2281-2308, doi:10.1038/nprot.2013.143 (2013).
76. Jiang, F. et al. Structures of a CRISPR-Cas9 R-loop complex primed for DNA cleavage. Science, doi:10.1126/science.aad8282 (2016).
77. Tsai, S. Q. et al. Dimeric CRISPR RNA-guided FokI nucleases for highly specific genome editing. Nat Biotech 32, 569-576, doi:10.1038/nbt.2908 (2014).
78. Lieber, M. R., Ma, Y., Pannicke, U. & Schwarz, K. Mechanism and regulation of human non-homologous DNA end-joining. Nat Rev Mol Cell Biol 4, 712-720 (2003).
79. Heller, R. C. & Marians, K. J. Replisome assembly and the direct restart of stalled replication forks. Nat Rev Mol Cell Biol 7, 932-943 (2006).
80. Pluciennik, A. et al. PCNA function in the activation and strand direction of MutLα endonuclease in mismatch repair. Proceedings of the National Academy of Sciences of the United States of America 107, 16066-16071, doi:10.1073/pnas.1010662107 (2010).
81. Seripa, D. et al. The missing ApoE allele. Annals of human genetics 71, 496-500, doi:10.1111/j.1469-1809.2006.00344.x (2007).
82. Kleinstiver, B. P. et al. High-fidelity CRISPR-Cas9 nucleases with no detectable genome-wide off-target effects. Nature 529, 490-495, doi:10.1038/nature16526 (2016).
83. Richardson, C. D., Ray, G. J., DeWitt, M. A., Curie, G. L. & Corn, J. E. Enhancing homology-directed genome editing by catalytically active and inactive CRISPR-Cas9 using asymmetric donor DNA. Nat Biotech 34, 339-344, doi:10.1038/nbt.3481 (2016).
84. Simonelli, V., Narciso, L., Dogliotti, E. & Fortini, P. Base excision repair intermediates are mutagenic in mammalian cells. Nucleic acids research 33, 4404-4411, doi:10.1093/nar/gki749 (2005).
85. Barnes, D. E. & Lindahl, T. Repair and Genetic Consequences of Endogenous DNA Base Damage in Mammalian Cells. Annual Review of Genetics 38, 445-476, doi:doi:10.1146/annurev.genet.38.072902.092448 (2004).

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein

LENGTHY TABLES
The patent application contains a lengthy table section. A copy of the table is available in electronic form from the USPTO web site (https://seqdata.uspto.gov/?pageRequest=docDetail&DocID=US20250270527A1). An electronic copy of the table will also be available from the USPTO upon request and payment of the fee set forth in 37 CFR 1.19(b)(3).

What is claimed is:

1. A fusion protein comprising: (i) a Cas9 domain; (ii) a cytidine deaminase domain; and (iii) a uracil glycosylase inhibitor (UGI) domain.

2. The fusion protein of claim 1, wherein the Cas9 domain comprises an amino acid sequence that is at least 85% identical to the amino acid sequence provided in SEQ ID NO: 674.

3. The fusion protein of claim 1, wherein the Cas9 domain is a Cas9 nickase domain that cuts a nucleotide target strand of a nucleotide duplex, wherein the nucleotide target strand is the strand that binds to a gRNA of the Cas9 nickase domain.

4. The fusion protein of claim 1, wherein the Cas9 domain is an nCas9 domain that comprises a D10A mutation in the amino acid sequence provided in SEQ ID NO: 10, or a corresponding mutation in any of the amino acid sequences provided in SEQ ID NOs: 11-260.

5. The fusion protein of claim 1, wherein the Cas9 domain is an nCas9 domain that comprises one or more of N496A, R660A, Q694A, and Q926A of the amino acid sequence provided in SEQ ID NO 10, or one or more corresponding mutations in any of the amino acid sequences provided in SEQ ID NOs: 11-260.

6. The fusion protein of claim 1, wherein the cytidine deaminase domain is a deaminase from the apolipoprotein B mRNA-editing complex (APOBEC) family deaminase.

7. The fusion protein of claim 6, wherein the APOBEC family deaminase is selected from the group consisting of APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, and APOBEC3H deaminase.

8. The fusion protein of claim 1, wherein the cytidine deaminase domain comprises an amino acid sequence that is at least 85% identical to an amino acid sequence of SEQ ID NO: 266-284, 607-610, 5724-5736, or 5738-5741.

9. The fusion protein of claim 1, wherein the cytidine deaminase domain comprises an amino acid sequence of SEQ ID NO: 266-284, 607-610, 5724-5736, or 5738-5741.

10. The fusion protein of claim 1, wherein the cytidine deaminase domain is a rat APOBEC1 (rAPOBEC1) deaminase comprising one or more mutations selected from the group consisting of W90Y, R126E, and R132E of SEQ ID NO: 284, or one or more corresponding mutations in another APOBEC deaminase.

11. The fusion protein of claim 1, wherein the cytidine deaminase domain is a human APOBEC1 (hAPOBEC1) deaminase comprising one or more mutations selected from the group consisting of W90Y, Q126E, and R132E of SEQ ID NO: 5724, or one or more corresponding mutations in another APOBEC deaminase.

12. The fusion protein of claim 1, wherein the cytidine deaminase domain is a human APOBEC3G (hAPOBEC3G) deaminase comprising one or more mutations selected from the group consisting of W285Y, R320E, and R326E of SEQ ID NO: 275, or one or more corresponding mutations in another APOBEC deaminase.

13. The fusion protein of claim 1, wherein the cytidine deaminase domain is an activation-induced deaminase (AID).

14. The fusion protein of claim 1, wherein the cytidine deaminase domain is a cytidine deaminase 1 from Petromyzon marinus (pmCDA1).

15. The fusion protein of claim 1, wherein the UGI domain comprises a domain capable if inhibiting UDG activity.

16. The fusion protein of claim 1, wherein the UGI domain comprises an amino acid sequence that is at least 85% identical to SEQ ID NO: 600.

17. The fusion protein of claim 1, wherein the UGI domain comprises an amino acid sequence as set forth in SEQ ID NO: 600.

18. The fusion protein of claim 1, wherein the fusion protein comprises the structure: NH₂-[cytidine deaminase domain]-[Cas9 domain]-[UGI domain]-COOH, and wherein each instance of “-” comprises an optional linker.

19. The fusion protein of claim 1, wherein the cytidine deaminase domain of (ii) and the nCas9 domain of (i) are linked via a linker comprising the amino acid sequence (GGGS)ₙ (SEQ ID NO: 265), (GGGGS)ₙ (SEQ ID NO: 5), (G)ₙ, (EAAAK)ₙ (SEQ ID NO: 6), (GGS)ₙ, (SGGS)ₙ(SEQ ID NO: 4288), SGSETPGTSESATPES (SEQ ID NO: 7), or (XP)ₙ motif, or a combination thereof, wherein n is independently an integer between 1 and 30, inclusive, and wherein X is any amino acid.

20. The fusion protein of claim 1, wherein the cytidine deaminase domain of (ii) and the nCas9 domain of (i) are linked via a linker comprising the amino acid sequence: SGSETPGTSESATPES (SEQ ID NO: 7).

21. The fusion protein of claim 1 further comprising a nuclear localization sequence (NLS).

22. The fusion protein of claim 21, wherein the NLS comprises the amino acid sequence PKKKRKV (SEQ ID NO: 741) or MDSLLMNRRKFLYQFKNVRWAKGRRETYLC (SEQ ID NO: 742)

23. The fusion protein of claim 21, wherein the fusion protein comprises the structure: NH₂-[cytidine deaminase domain]-[nCas9 domain]-[UGI domain]-[NLS]-COOH, and wherein each instance of “-” comprises an optional linker.

24. The fusion protein of claim 21, wherein the UGI domain and the NLS are linked via a linker comprising the amino acid sequence: SGGS (SEQ ID NO: 4288), or wherein the nCas9 domain and the UGI domain are linked via a linker comprising the amino acid sequence: SGGS (SEQ ID NO: 4288).

25. The fusion protein of claim 1, wherein the fusion protein comprises the amino acid sequence set forth in SEQ ID NO: 594.

26. A complex comprising the fusion protein of claim 1 and a guide RNA bound to the nCas9 domain of the fusion protein.

27. A method comprising contacting a nucleic acid molecule with the fusion protein of claim 1 and a guide RNA, wherein the guide RNA comprises a sequence of at least 10 contiguous nucleotides that is complementary to a target sequence in the genome of an organism and comprises a target base pair.

28. The method of claim 27, wherein the target base pair comprises a T to C point mutation associated with a disease or disorder, and wherein the deamination of the mutant C base results in a sequence that is not associated with a disease or disorder.

29. The method of claim 27, wherein the contacting results in less than 20% indel formation upon base editing.

30. The method of claim 27, wherein the contacting results in at least 2:1 intended to unintended product upon base editing.

31. A fusion protein comprising: (i) a nuclease-inactive Cas9 (dCas9) domain and (ii) an apolipoprotein B mRNA-editing complex 1 (APOBEC1) deaminase domain, wherein the deaminase domain is fused to the N-terminus of the dCas9 domain via a linker comprising the amino acid sequence SGSETPGTSESATPES (SEQ ID NO: 7).

32. The fusion protein of claim 31, wherein the nuclease-inactive Cas9 (dCas9) domain of (i) comprises the amino acid sequence that is at least 85% identical to the amino acid sequence set forth in SEQ ID NO: 263.

33. The fusion protein of claim 31, wherein the nuclease-inactive Cas9 (dCas9) domain of (i) comprises the amino acid sequence set forth in SEQ ID NO: 263.

34. The fusion protein of any one of claims 31-33, wherein the deaminase is a rat APOBEC1 deaminase that is at least 85% identical the amino acid sequence as set forth in (SEQ ID NO: 284).

35. The fusion protein of any one of claims 31-34, wherein the deaminase is rat APOBEC1 deaminase comprising the amino acid sequence as set forth in (SEQ ID NO: 284).

36. The fusion protein of any one of claims 31-33, wherein the deaminase is a human APOBEC1 deaminase that is at least 85% identical to the amino acid sequence as set forth in (SEQ ID NO: 282).

37. The fusion protein of any one of claims 31-33, wherein the deaminase is a human APOBEC1 deaminase comprising the amino acid sequence as set forth in (SEQ ID NO: 282).

38. The fusion protein of any one of claims 31-37, wherein the UGI domain comprises an amino acid sequence that is at least 80%, at least 85%, at least 90%, at least 92%, at least 95%, at least 96%, at least 97%, at least 98%, at least 99%, or at least 99.5% identical to SEQ ID NO: 600.

39. The fusion protein of any one of claims 31-38, wherein the UGI domain comprises the amino acid sequence as set forth in SEQ ID NO: 600

40. The fusion protein of any one of claims 31-37, wherein the UGI domain comprises an amino acid sequence that is at least 80%, at least 85%, at least 90%, at least 92%, at least 95%, at least 96%, at least 97%, at least 98%, at least 99%, or at least 99.5% identical to any one of SEQ ID NOs: 322-324.

41. The fusion protein of any one of claims 31-37, wherein the UGI domain comprises the amino acid sequence as set forth in any one of SEQ ID NOs: 322-324.

42. The fusion protein of any one of claims 31-41, wherein the fusion protein comprises amino acid residues 11-1629 of the amino acid sequence set forth in SEQ ID NO: 591.

43. The fusion protein of any one of claims 31-41, wherein the fusion protein comprises the amino acid sequence set forth in any one of SEQ ID NOs: 591-593, 611, 612, 615, 657, 658, and 5737.

44. A fusion protein comprising: (i) a nuclease-inactive Cas9 (dCas9) domain; (ii) a nucleic acid editing domain; and (iii) a uracil glycosylase inhibitor (UGI) domain.

45. The fusion protein of claim 44, wherein the amino acid sequence of the dCas9 domain comprises a D10X mutation of the amino acid sequence provided in SEQ ID NO: 10, or a corresponding mutation in any of the amino acid sequences provided in SEQ ID NOs: 11-260, wherein X is any amino acid except for D.

46. The fusion protein of claim 44 or 45, wherein the amino acid sequence of the dCas9 domain comprises a D10A mutation of the amino acid sequence provided in SEQ ID NO: 10, or a corresponding mutation in any of the amino acid sequences provided in SEQ ID NOs: 11-260.

47. The fusion protein of any one of claims 44-46, wherein the amino acid sequence of the dCas9 domain comprises an H840X mutation of the amino acid sequence provided in SEQ ID NO: 10, or a corresponding mutation in any of the amino acid sequences provided in SEQ ID NOs: 11-260, wherein X is any amino acid except for H.

48. The fusion protein of any one of claims 44-47, wherein the amino acid sequence of the dCas9 domain comprises an H840A mutation of the amino acid sequence provided in SEQ ID NO: 10, or a corresponding mutation in any of the amino acid sequences provided in SEQ ID NOs: 11-260.

49. The fusion protein of any one of claims 44-48, wherein the dCas9 domain comprises an amino acid sequence that is at least 85% identical to the amino acid sequence as set forth in SEQ ID NO: 263.

50. The fusion protein of any one of claims 44-49, wherein the dCas9 domain comprises the amino acid sequence as set forth in SEQ ID NO: 263.

51. The fusion protein of any one of claims 44-50, wherein the dCas9 domain comprises one or more of a N497X, R661X, Q695X, and Q926X mutation of the amino acid sequence provided in SEQ ID NO: 10, or a corresponding mutation in any of the amino acid sequences provided in SEQ ID NOs: 11-260, wherein X is any amino acid.

52. The fusion protein of any one of claims 44-51, wherein the dCas9 domain comprises one or more of a N497A, R661A, Q695A, and Q926A mutation of the amino acid sequence provided in SEQ ID NO: 10, or a corresponding mutation in any of the amino acid sequences provided in SEQ ID NOs: 11-260.

53. The fusion protein of any one of claims 44-52, wherein the dCas9 domain comprises a N497A, R661A, Q695A, and Q926A mutation of the amino acid sequence provided in SEQ ID NO: 10, or a corresponding mutation in any of the amino acid sequences provided in SEQ ID NOs: 11-260.

54. The fusion protein of any one of claims 44-53, wherein the dCas9 domain comprises a Staphylococcus aureus (SaCas9).

55. The fusion protein of claim 54, wherein the SaCas9 comprises the amino acid sequence SEQ ID NO: 4273.

56. The fusion protein of claim 54 or 55, wherein the SaCas9 domain comprises one or more of a E781K, N967K, or R1014H mutation of SEQ ID NO: 4273, or one or more corresponding mutation in any of the amino acid sequences provided in SEQ ID NOs: 11-260.

57. The fusion protein of any one of claims 44-53, wherein the dCas9 domain comprises one or more of a D1134E, R1334Q, and T1336R mutation of SEQ ID NO: 4276, or a corresponding mutation in any of the amino acid sequences provided in SEQ ID NOs: 11-260.

58. The fusion protein of any one of claims 44-53, wherein the dCas9 domain comprises one or more of a D1134V, R1334Q, and T1336R mutation of SEQ ID NO: 4276, or a corresponding mutation in any of the amino acid sequences provided in SEQ ID NOs: 11-260.

59. The fusion protein of any one of claims 44-53, wherein the dCas9 domain comprises one or more of a D1134V, G1217R, R1334Q, and T1336R mutation of SEQ ID NO: 4276, or a corresponding mutation in any of the amino acid sequences provided in SEQ ID NOs: 11-260.

60. The fusion protein of any of claims 44-59, wherein the nucleic acid editing domain is fused to the N-terminus of the dCas9 domain.

61. The fusion protein of any one of claims 44-60, wherein the UGI domain is fused to the C-terminus of the dCas9 domain.

62. The fusion protein of any one of claims 44-61, wherein the dCas9 domain and the nucleic acid editing domain are fused via a linker.

63. The fusion protein of any one of claims 44-62, wherein the dCas9 domain and the UGI domain are fused via a linker.

64. The fusion protein of claim 62 or 63, wherein the linker comprises the amino acid sequence (GGGGS)ₙ (SEQ ID NO: 5), (G)ₙ, (EAAAK)ₙ (SEQ ID NO: 6), (GGS)ₙ, SGSETPGTSESATPES (SEQ ID NO: 7), SGGS (SEQ ID NO: 4288), (XP)ₙ, or any combination thereof, wherein n is independently an integer between 1 and 30, and wherein X is any amino acid.

65. The fusion protein of claim 62 or 63, wherein the linker comprises a covalent bond.

66. The fusion protein of claim 64, wherein the linker comprises the amino acid sequence (GGS)ₙ, wherein n is 1, 3, or 7.

67. The fusion protein of claim 64, wherein the linker comprises the amino acid sequence SGSETPGTSESATPES (SEQ ID NO: 7).

68. The fusion protein of claim 62, wherein the dCas9 domain and the nucleic acid editing domain are fused via a linker comprising the amino acid sequence SGSETPGTSESATPES (SEQ ID NO: 7).

69. The fusion protein of claim 62, wherein the dCas9 domain and the nucleic acid editing domain are fused via a linker comprising the amino acid sequence (GGS)ₙ, wherein n is 1, 3, or 7.

70. The fusion protein of claim 63, wherein the dCas9 domain and the UGI domain are fused via a linker comprising the amino acid sequence (GGGGS)n (SEQ ID NO: 5), (G)n, (EAAAK)n (SEQ ID NO: 6), (GGS)n, SGSETPGTSESATPES (SEQ ID NO: 7), SGGS (SEQ ID NO: 4288), (XP)n, or any combination thereof, wherein n is independently an integer between 1 and 30, and wherein X is any amino acid.

71. The fusion protein of claim 63, wherein the dCas9 domain and the UGI domain are fused via a linker comprising the amino acid sequence SGGS (SEQ ID NO: 4288).

72. The fusion protein of any one of claims 44-71, wherein the fusion protein comprises the structure [nucleic acid editing domain]-[optional linker]-[dCas9 domain]-[optional linker]-[UGI].

73. The fusion protein of any one of claims 44-67 wherein the fusion protein comprises the structure [nucleic acid editing domain]-[optional linker]-[UGI]-[optional linker]-[dCas9]; [UGI]-[optional linker]-[nucleic acid editing domain]-[optional linker]-[dCas9]; [UGI]-[optional linker]-[dCas9]-[optional linker]-[nucleic acid editing domain]; [dCas9]-[optional linker]-[UGI]-[optional linker]-[nucleic acid editing domain]; or [dCas9]-[optional linker]-[nucleic acid editing domain]-[optional linker]-[UGI].

74. The fusion protein of any one of claims 44-73, wherein the nucleic acid editing domain comprises a deaminase.

75. The fusion protein of claim 74 wherein the deaminase is a cytidine deaminase.

76. The fusion protein of claim 74 or 75, wherein the deaminase is an apolipoprotein B mRNA-editing complex (APOBEC) family deaminase.

77. The fusion protein of any one of claims 74-76, wherein the deaminase is an APOBEC1 deaminase.

78. The fusion protein of any one of claims 74-76, wherein the deaminase is an APOBEC2 deaminase.

79. The fusion protein of any one of claims 74-76, wherein the deaminase is an APOBEC3A deaminase.

80. The fusion protein of any one of claims 74-76, wherein the deaminase is an APOBEC3B deaminase.

81. The fusion protein of any one of claims 74-76, wherein the deaminase is an APOBEC3C deaminase.

82. The fusion protein of any one of claims 74-76, wherein the deaminase is an APOBEC3D deaminase.

83. The fusion protein of any one of claims 74-76, wherein the deaminase is an APOBEC3F deaminase.

84. The fusion protein of any one of claims 74-76, wherein the deaminase is an APOBEC3G deaminase.

85. The fusion protein of any one of claims 74-76, wherein the deaminase is an APOBEC3H deaminase.

86. The fusion protein of any one of claims 74-76, wherein the deaminase is an APOBEC4 deaminase.

87. The fusion protein of claim 74 or 75, wherein the deaminase is an activation-induced deaminase (AID).

88. The fusion protein of claim 74 or 75, wherein the deaminase is an APOBEC deaminase comprising one or more mutations selected from the group consisting of H121R, H122R, R126A, R126E, R118A, W90A, W90Y, and R132E of rAPOBEC1 (SEQ ID NO: 284), or one or more corresponding mutations in another APOBEC deaminase.

89. The fusion protein of claim 74 or 75, wherein the deaminase is an APOBEC deaminase comprising a W90Y, a R126E, and a R132E mutation of rAPOBEC1 (SEQ ID NO: 284), or one or more corresponding mutations in another APOBEC deaminase.

90. The fusion protein of claim 74 or 75, wherein the deaminase comprises one or more mutations selected from the group consisting of D316R, D317R, R320A, R320E, R313A, W285A, W285Y, R326E of hAPOBEC3G (SEQ ID NO: 275), or one or more corresponding mutations in another APOBEC deaminase.

91. The fusion protein of claim 74 or 75, wherein the deaminase is an APOBEC deaminase comprising a W285Y, a R320E, and a R326E mutation of hAPOBEC3G (SEQ ID NO: 275), or one or more corresponding mutations in another APOBEC deaminase.

92. The fusion protein of any one of claims 74-91, wherein the deaminase is from a human, chimpanzee, gorilla, monkey, cow, dog, rat, or mouse.

93. The fusion protein of any one of claims 74-92, wherein the deaminase is from a human.

94. The fusion protein of any one of claims 74-92, wherein the deaminase is from a rat.

95. The fusion protein of claim 74 or 75, wherein the deaminase is an cytidine deaminase 1 from Petromyzon marinus (pmCDA1).

96. The fusion protein of any one of claims 74-76, wherein the deaminase is a rat APOBEC1 deaminase comprising the amino acid sequence set forth in (SEQ ID NO: 284).

97. The fusion protein of any one of claims 74-76, wherein the deaminase is a human APOBEC1 deaminase comprising the amino acid sequence set forth in (SEQ ID NO: 282).

98. The fusion protein of claim 95, wherein the pmCDA1 comprises an amino acid sequence set forth in (SEQ ID NO: 5738).

99. The fusion protein of claim 84, wherein the APOBEC3G is a human APOBEC3G comprising the amino acid sequence set forth in (SEQ ID NO: 275).

100. The fusion protein of claim 84, wherein the APOBEC3G is a human APOBEC3G variant comprising the amino acid sequence set forth in any one of (SEQ ID NOs: 5739-5741).

101. The fusion protein of claim 74 or 75, wherein the deaminase is at least 80%, at least 85%, at least 90%, at least 92%, at least 95%, at least 96%, at least 97%, at least 98%, at least 99%, or at least 99.5% identical to any one of the amino acid sequences set forth in SEQ ID NOs: 266-284, 607-610, 5724-5736, and 5738-5741.

102. The fusion protein of claim 74 or 75, wherein the deaminase comprises the amino acid sequence set forth in any one of SEQ ID NOs: 266-284, 607-610, 5724-5736, and 5738-5741.

103. The fusion protein of any one of claims 44-102, wherein the UGI domain comprises an amino acid sequence that is at least 80%, at least 85%, at least 90%, at least 92%, at least 95%, at least 96%, at least 97%, at least 98%, at least 99%, or at least 99.5% identical to SEQ ID NO: 600.

104. The fusion protein of any one of claims 44-103, wherein the UGI domain comprises the amino acid sequence as set forth in SEQ ID NO: 600

105. The fusion protein of any one of claims 44-102, wherein the UGI domain comprises an amino acid sequence that is at least 80%, at least 85%, at least 90%, at least 92%, at least 95%, at least 96%, at least 97%, at least 98%, at least 99%, or at least 99.5% identical to any one of SEQ ID NOs: 322-324.

106. The fusion protein of any one of claims 44-102, wherein the UGI domain comprises the amino acid sequence as set forth in any one of SEQ ID NOs: 322-324.

107. A fusion protein comprising: (i) a Cas9 nickase domain and (ii) an apolipoprotein B mRNA-editing complex 1 (APOBEC1) deaminase domain, wherein the deaminase domain is fused to the N-terminus of the Cas9 nickase domain via a linker comprising the amino acid sequence SGSETPGTSESATPES (SEQ ID NO: 7).

108. The fusion protein of claim 107, wherein the deaminase is rat APOBEC1 (SEQ ID NO: 284).

109. The fusion protein of claim 107 or 108, wherein the deaminase is human APOBEC1 (SEQ ID NO: 282).

110. A fusion protein comprising: (i) a Cas9 nickase domain and (ii) an apolipoprotein B mRNA-editing complex 3G (APOBEC3G) deaminase domain, wherein the deaminase domain is fused to the N-terminus of the Cas9 nickase domain via a linker comprising the amino acid sequence SGSETPGTSESATPES (SEQ ID NO: 7).

111. The fusion protein of claim 110, wherein the deaminase is a human APOBEC3G deaminase comprising an amino acid sequence at least 85% identical to the amino acid sequence set forth in (SEQ ID NO: 275).

112. The fusion protein of claim 110 or 111, wherein the deaminase is a human APOBEC3G (SEQ ID NO: 275).

113. The fusion protein of claim 110, wherein the APOBEC3G is a human APOBEC3G variant comprising an amino acid sequence that is at least 85% identical to the amino acid sequence as set forth in any one of (SEQ ID NOs: 5739-5741).

114. The fusion protein of claim 110, wherein the APOBEC3G is a human APOBEC3G variant comprising the amino acid sequence set forth in any one of (SEQ ID NOs: 5739-5741).

115. A fusion protein comprising: (i) a Cas9 nickase domain and (ii) pmCDA1 domain, wherein the deaminase domain is fused to the N-terminus of the Cas9 nickase domain via a linker comprising the amino acid sequence SGSETPGTSESATPES (SEQ ID NO: 7).

116. The fusion protein of claim 115, wherein the pmCDA1 comprises an amino acid sequence that is at least 85% identical to the amino acid sequence as set forth in (SEQ ID NO: 5738).

117. The fusion protein of claim 115 or 116, wherein the pmCDA1 comprises an amino acid sequence set forth in (SEQ ID NO: 5738).

118. The fusion protein of any one of claims 107-117, wherein the amino acid sequence of the Cas9 nickase domain comprises a D10X mutation of the amino acid sequence provided in SEQ ID NO: 10, or a corresponding mutation in any of the amino acid sequences provided in SEQ ID NOs: 11-260, wherein X is any amino acid except for D.

119. The fusion protein of any one of claims 107-118, wherein the amino acid sequence of the Cas9 nickase domain comprises a D10A mutation of the amino acid sequence provided in SEQ ID NO: 10, or a corresponding mutation in any of the amino acid sequences provided in SEQ ID NOs: 11-260.

120. The fusion protein of any one of claims 107-119, wherein the amino acid sequence of the Cas9 nickase domain comprises a histidine at amino acid position 840 of the amino acid sequence provided in SEQ ID NO: 10, or a corresponding amino acid position in any of the amino acid sequences provided in SEQ ID NOs: 11-260.

121. The fusion protein of any one of claims 107-120, wherein the amino acid sequence of the Cas9 nickase domain comprises the amino acid sequence that is at least 85% identical to the amino acid sequence as set forth in SEQ ID NO: 674.

122. The fusion protein of any one of claims 107-121, wherein the amino acid sequence of the Cas9 nickase domain comprises the amino acid sequence as set forth in SEQ ID NO: 674.

123. The fusion protein of any one of claims 107-122, wherein the UGI domain comprises an amino acid sequence that is at least 80%, at least 85%, at least 90%, at least 92%, at least 95%, at least 96%, at least 97%, at least 98%, at least 99%, or at least 99.5% identical to SEQ ID NO: 600.

124. The fusion protein of any one of claims 107-123, wherein the UGI domain comprises the amino acid sequence as set forth in SEQ ID NO: 600

125. The fusion protein of any one of claims 107-122, wherein the UGI domain comprises an amino acid sequence that is at least 80%, at least 85%, at least 90%, at least 92%, at least 95%, at least 96%, at least 97%, at least 98%, at least 99%, or at least 99.5% identical to any one of SEQ ID NOs: 322-324.

126. The fusion protein of any one of claims 107-122, wherein the UGI domain comprises the amino acid sequence as set forth in any one of SEQ ID NOs: 322-324.

127. The fusion protein of any one of claims 107-126, wherein the fusion protein comprises the amino acid sequence set forth in any one of SEQ ID NOs: 594, 5743, 5745, and 5746.

128. A fusion protein comprising: (i) a Cas9 nickase (nCas9) domain; (ii) a nucleic acid editing domain; and (iii) a uracil glycosylase inhibitor (UGI) domain.

129. The fusion protein of claim 128, wherein the amino acid sequence of the Cas9 nickase domain comprises a D10X mutation of the amino acid sequence provided in SEQ ID NO: 10, or a corresponding mutation in any of the amino acid sequences provided in SEQ ID NOs: 11-260, wherein X is any amino acid except for D.

130. The fusion protein of claim 128 or 129, wherein the amino acid sequence of the Cas9 nickase domain comprises a D10A mutation of the amino acid sequence provided in SEQ ID NO: 10, or a corresponding mutation in any of the amino acid sequences provided in SEQ ID NOs: 11-260.

131. The fusion protein of any one of claims 128-130, wherein the amino acid sequence of the Cas9 nickase domain comprises a histidine at amino acid position 840 of the amino acid sequence provided in SEQ ID NO: 10, or a corresponding amino acid position in any of the amino acid sequences provided in SEQ ID NOs: 11-260.

132. The fusion protein of any one of claims 128-131, wherein the amino acid sequence of the Cas9 nickase domain comprises an amino acid sequence that is at least 85% identical to the amino acid sequence as set forth in SEQ ID NO: 674.

133. The fusion protein of any one of claims 128-131, wherein the amino acid sequence of the Cas9 nickase domain comprises the amino acid sequence as set forth in SEQ ID NO: 674.

134. The fusion protein of any one of claims 128-133, wherein the Cas9 nickase domain comprises one or more of a N497X, R661X, Q695X, and Q926X mutation of the amino acid sequence provided in SEQ ID NO: 10, or a corresponding mutation in any of the amino acid sequences provided in SEQ ID NOs: 11-260, wherein X is any amino acid.

135. The fusion protein of any one of claims 128-134, wherein the Cas9 nickase domain comprises one or more of a N497A, R661A, Q695A, and Q926A mutation of the amino acid sequence provided in SEQ ID NO: 10, or a corresponding mutation in any of the amino acid sequences provided in SEQ ID NOs: 11-260.

136. The fusion protein of any one of claims 128-135, wherein the Cas9 nickase domain comprises a N497A, R661A, Q695A, and Q926A mutation of the amino acid sequence provided in SEQ ID NO: 10, or a corresponding mutation in any of the amino acid sequences provided in SEQ ID NOs: 11-260.

137. The fusion protein of any one of claims 128-136, wherein the Cas9 nickase domain comprises a Staphylococcus aureus (SaCas9).

138. The fusion protein of claim 137, wherein the SaCas9 comprises the amino acid sequence SEQ ID NO: 4273.

139. The fusion protein of claim 137 or 138, wherein the SaCas9 comprises one or more of a E781K, N967K, or R1014H mutation of SEQ ID NO: 4273, or one or more corresponding mutation in any of the amino acid sequences provided in SEQ ID NOs: 11-260.

140. The fusion protein of any one of claims 128-136, wherein the dCas9 domain comprises one or more of a D1134E, R1334Q, and T1336R mutation of SEQ ID NO: 4276, or a corresponding mutation in any of the amino acid sequences provided in SEQ ID NOs: 11-260.

141. The fusion protein of any one of claims 128-136, wherein the dCas9 domain comprises one or more of a D1134V, R1334Q, and T1336R mutation of SEQ ID NO: 4276, or a corresponding mutation in any of the amino acid sequences provided in SEQ ID NOs: 11-260.

142. The fusion protein of any one of claims 128-136, wherein the dCas9 domain comprises one or more of a D1134V, G1217R, R1334Q, and T1336R mutation of SEQ ID NO: 4276, or a corresponding mutation in any of the amino acid sequences provided in SEQ ID NOs: 11-260.

143. The fusion protein of any of claims 128-142, wherein the nucleic acid editing domain is fused to the N-terminus of the Cas9 nickase domain.

144. The fusion protein of any one of claims 128-143, wherein the UGI domain is fused to the C-terminus of the Cas9 nickase domain.

145. The fusion protein of any one of claims 128-144, wherein the Cas9 nickase domain and the nucleic acid editing domain are fused via a linker.

146. The fusion protein of any one of claims 128-145, wherein the Cas9 nickase domain and the UGI domain are fused via a linker.

147. The fusion protein of claims 145 or 146, wherein the linker comprises the amino acid sequence (GGGGS)n (SEQ ID NO: 5), (G)n, (EAAAK)n (SEQ ID NO: 6), (GGS)n, SGSETPGTSESATPES (SEQ ID NO: 7), SGGS (SEQ ID NO: 4288), (XP)n, or any combination thereof, wherein n is independently an integer between 1 and 30, and wherein X is any amino acid.

148. The fusion protein of claim 145 or 146, wherein the linker comprises a covalent bond.

149. The fusion protein of claim 147, wherein the linker comprises the amino acid sequence (GGS)n, wherein n is 1, 3, or 7.

150. The fusion protein of claim 147, wherein the linker comprises the amino acid sequence SGSETPGTSESATPES (SEQ ID NO: 7).

151. The fusion protein of claim 145, wherein the nCas9 domain and the nucleic acid editing domain are fused via a linker comprising the amino acid sequence SGSETPGTSESATPES (SEQ ID NO: 7).

152. The fusion protein of claim 145, wherein the nCas9 domain and the nucleic acid editing domain are fused via a linker comprising the amino acid sequence (GGS)n, wherein n is 1, 3, or 7.

153. The fusion protein of claim 146, wherein the nCas9 domain and the UGI domain are fused via a linker comprising the amino acid sequence (GGGGS)n (SEQ ID NO: 5), (G)n, (EAAAK)n (SEQ ID NO: 6), (GGS)n, SGSETPGTSESATPES (SEQ ID NO: 7), SGGS (SEQ ID NO: 4288), (XP)n, or any combination thereof, wherein n is independently an integer between 1 and 30, and wherein X is any amino acid.

154. The fusion protein of claim 146, wherein the nCas9 domain and the UGI domain are fused via a linker comprising the amino acid sequence SGGS (SEQ ID NO: 4288).

155. The fusion protein of any one of claims 128-154 wherein the fusion protein comprises the structure [nucleic acid editing domain]-[optional linker]-[Cas9 nickase]-[optional linker]-[UGI domain].

156. The fusion protein of any one of claims 128-154 wherein the fusion protein comprises the structure [nucleic acid editing domain]-[optional linker]-[UGI domain]-[optional linker]-[Cas9 nickase]; [UGI domain]-[optional linker]-[nucleic acid editing domain]-[optional linker]-[Cas9 nickase]; [UGI domain]-[optional linker]-[Cas9 nickase]-[optional linker]-[nucleic acid editing domain]; [Cas9 nickase]-[optional linker]-[UGI domain]-[optional linker]-[nucleic acid editing domain]; or [Cas9 nickase]-[optional linker]-[nucleic acid editing domain]-[optional linker]-[UGI domain].

157. The fusion protein of any one of claims 128-156, wherein the nucleic acid editing domain comprises a deaminase.

158. The fusion protein of claim 157 wherein the deaminase is a cytidine deaminase.

159. The fusion protein of claim 157 or 158, wherein the deaminase is an apolipoprotein B mRNA-editing complex (APOBEC) family deaminase.

160. The fusion protein of any one of claims 157-159, wherein the deaminase is an APOBEC1 deaminase.

161. The fusion protein of any one of claims 157-159, wherein the deaminase is an APOBEC2 deaminase.

162. The fusion protein of any one of claims 157-159, wherein the deaminase is an APOBEC3A deaminase.

163. The fusion protein of any one of claims 157-159, wherein the deaminase is an APOBEC3B deaminase.

164. The fusion protein of any one of claims 157-159, wherein the deaminase is an APOBEC3C deaminase.

165. The fusion protein of any one of claims 157-159, wherein the deaminase is an APOBEC3D deaminase.

166. The fusion protein of any one of claims 157-159, wherein the deaminase is an APOBEC3F deaminase.

167. The fusion protein of any one of claims 157-159, wherein the deaminase is an APOBEC3G deaminase.

168. The fusion protein of any one of claims 157-159, wherein the deaminase is an APOBEC3H deaminase.

169. The fusion protein of any one of claims 157-159, wherein the deaminase is an APOBEC4 deaminase.

170. The fusion protein of claim 157 or 158, wherein the deaminase is an activation-induced deaminase (AID).

171. The fusion protein of claim 157 or 158, wherein the deaminase is an APOBEC deaminase comprising one or more mutations selected from the group consisting of H121R, H122R, R126A, R126E, R118A, W90A, W90Y, and R132E of rAPOBEC1 (SEQ ID NO: 284), or one or more corresponding mutations in another APOBEC deaminase.

172. The fusion protein of claim 157 or 158, wherein the deaminase is an APOBEC deaminase comprising a W90Y, a R126E, and a R132E mutation of rAPOBEC1 (SEQ ID NO: 284), or one or more corresponding mutations in another APOBEC deaminase.

173. The fusion protein of claim 157 or 158, wherein the deaminase comprises one or more mutations selected from the group consisting of D316R, D317R, R320A, R320E, R313A, W285A, W285Y, R326E of hAPOBEC3G (SEQ ID NO: 275), or one or more corresponding mutations in another APOBEC deaminase.

174. The fusion protein of claim 157 or 158, wherein the deaminase is an APOBEC deaminase comprising a W285Y, a R320E, and a R326E mutation of hAPOBEC3G (SEQ ID NO: 275), or one or more corresponding mutations in another APOBEC deaminase.

175. The fusion protein of any one of claims 157-174, wherein the deaminase is from a human, chimpanzee, gorilla, monkey, cow, dog, rat, or mouse.

176. The fusion protein of any one of claims 157-175, wherein the deaminase is from a human.

177. The fusion protein of any one of claims 157-175, wherein the deaminase is from a rat.

178. The fusion protein of claim 157 or 158, wherein the deaminase is an cytidine deaminase 1 from Petromyzon marinus (pmCDA1).

179. The fusion protein of any one of claims 157-159, wherein the deaminase is a rat APOBEC1 deaminase comprising the amino acid sequence set forth in (SEQ ID NO: 284).

180. The fusion protein of any one of claims 157-159, wherein the deaminase is a human APOBEC1 deaminase comprising the amino acid sequence set forth in (SEQ ID NO: 282).

181. The fusion protein of claim 178, wherein the pmCDA1 comprises an amino acid sequence set forth in (SEQ ID NO: 5738).

182. The fusion protein of claim 167, wherein the APOBEC3G is a human APOBEC3G comprising the amino acid sequence set forth in (SEQ ID NO: 275).

183. The fusion protein of claim 167, wherein the APOBEC3G is a human APOBEC3G variant comprising the amino acid sequence set forth in any one of (SEQ ID NO: 5739-5741).

184. The fusion protein of claim 157 or 158, wherein the deaminase is at least 80%, at least 85%, at least 90%, at least 92%, at least 95%, at least 96%, at least 97%, at least 98%, at least 99%, or at least 99.5% identical to any one of the amino acid sequences set forth in SEQ ID NOs: 266-284, 607-610, 5724-5736 and 5738-5741.

185. The fusion protein of claim 157 or 158, wherein the deaminase comprises the amino acid sequence set forth in any one of SEQ ID NOs: 266-284, 607-610, 5724-5736 and 5738-5741.

186. The fusion protein of any one of claims 128-185, wherein the UGI domain comprises an amino acid sequence that is at least 80%, at least 85%, at least 90%, at least 92%, at least 95%, at least 96%, at least 97%, at least 98%, at least 99%, or at least 99.5% identical to SEQ ID NO: 600.

187. The fusion protein of any one of claims 128-186, wherein the UGI domain comprises the amino acid sequence as set forth in SEQ ID NO: 600.

188. The fusion protein of any one of claims 128-185, wherein the UGI domain comprises an amino acid sequence that is at least 80%, at least 85%, at least 90%, at least 92%, at least 95%, at least 96%, at least 97%, at least 98%, at least 99%, or at least 99.5% identical to any one of SEQ ID NOs: 322-324.

189. The fusion protein of any one of claims 128-185, wherein the UGI domain comprises the amino acid sequence as set forth in any one of SEQ ID NOs: 322-324.

190. A complex comprising the fusion protein of anyone of claims 1-30, and a guide RNA (gRNA) bound to the Cas9 domain of the fusion protein.

191. A complex comprising the fusion protein of any one of claims 31-106, and a guide RNA (gRNA) bound to the dCas9 domain of the fusion protein.

192. A complex comprising the fusion protein of any one of claims 107-189 and a guide RNA (gRNA) bound to the Cas9 nickase (nCas9) domain of the fusion protein.

193. The complex of any one of claims 190-192, wherein the guide RNA is from 15-100 nucleotides long and comprises a sequence of at least 10 contiguous nucleotides that is complementary to a target sequence.

194. The complex of claim 193, wherein the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long.

195. The complex of any one of claims 190-194, wherein the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence.

196. The complex of any one of claims 190-195, wherein the target sequence is a DNA sequence.

197. The complex of claim 196, wherein the target sequence is in the genome of an organism.

198. The complex of claim 197, wherein the organism is a prokaryote.

199. The complex of claim 198, wherein the prokaryote is bacteria.

200. The complex of claim 197, wherein the organism is a eukaryote.

201. The complex of claim 200, wherein the organism is a plant.

202. The complex of claim 200, wherein the organism is a vertebrate.

203. The complex of claim 202, wherein the vertebrate is a mammal.

204. The complex of claim 203, wherein the mammal is a mouse or rat.

205. The complex of claim 203, wherein the mammal is human.

206. A method comprising contacting a nucleic acid molecule with the fusion protein of any one of claims 1-189 and a guide RNA, wherein the guide RNA is from 15-100 nucleotides long and comprises a sequence of at least 10 contiguous nucleotides that is complementary to a target sequence.

207. A method comprising contacting a nucleic acid molecule with the complex of any one of claims 190-205.

208. The method of claim 206 or 207, wherein the nucleic acid is DNA.

209. The method of claim 208, wherein the nucleic acid is double-stranded DNA.

210. The method of any one of claims 206-209, wherein the target sequence comprises a sequence associated with a disease or disorder.

211. The method of claim 210, wherein the target sequence comprises a point mutation associated with a disease or disorder.

212. The method of claim 211, wherein the activity of the fusion protein, or the complex results in a correction of the point mutation.

213. The method of any one of claims 206-212, wherein the target sequence comprises a T to C point mutation associated with a disease or disorder, and wherein the deamination of the mutant C base results in a sequence that is not associated with a disease or disorder.

214. The method of claim 213, wherein the target sequence encodes a protein, and wherein the point mutation is in a codon and results in a change in the amino acid encoded by the mutant codon as compared to a wild-type codon.

215. The method of claim 214, wherein the deamination of the mutant C results in a change of the amino acid encoded by the mutant codon.

216. The method of claim 215, wherein the deamination of the mutant C results in the codon encoding a wild-type amino acid.

217. The method of any one of claims 206-216, wherein the contacting is performed in vivo in a subject.

218. The method of any one of claims 206-216, wherein the contacting is performed in vitro.

219. The method of claim 217, wherein the subject has been diagnosed with a disease or disorder.

220. The method of any one of claims 210-219, wherein the disease or disorder is cystic fibrosis, phenylketonuria, epidermolytic hyperkeratosis (EHK), Charcot-Marie-Toot disease type 4J, neuroblastoma (NB), von Willebrand disease (vWD), myotonia congenital, hereditary renal amyloidosis, dilated cardiomyopathy (DCM), hereditary lymphedema, familial Alzheimer's disease, HIV, Prion disease, chronic infantile neurologic cutaneous articular syndrome (CINCA), desmin-related myopathy (DRM), a neoplastic disease associated with a mutant PI3KCA protein, a mutant CTNNB1 protein, a mutant HRAS protein, or a mutant p53 protein.

221. The method of any one of claims 211-220, wherein the disease or disorder is associated with a T>C or A>G mutation in a gene selected from the genes disclosed in Table 1.

222. The method of any one of claims 211-220, wherein the disease or disorder is associated with a T>C or A>G mutation in a gene selected from the genes disclosed in Table 2 or 3.

223. The method of any one of claims 206-222, wherein the guide RNA comprises a nucleotide sequence of any one of the protospacer sequences in Table 2 or Table 3.

224. A method for editing a nucleobase pair of a double-stranded DNA sequence, the method comprising:
a. contacting a target region of the double-stranded DNA sequence with a complex comprising a nucleobase editor and a guide nucleic acid, wherein the target region comprises a target nucleobase pair;
b. inducing strand separation of said target region;
c. converting a first nucleobase of said target nucleobase pair in a single strand of the target region to a second nucleobase; and
d. cutting no more than one strand of said target region;
wherein a third nucleobase complementary to the first nucleobase base is replaced by a fourth nucleobase complementary to the second nucleobase and the method causes less than 20% indel formation in the double-stranded DNA sequence.

225. The method of claim 224, wherein the method causes less than 20%, 19%, 18%, 16%, 14%, 12%, 10%, 8%, 6%, 4%, 2%, or 1% indel formation.

226. The method of claim 224 or 225, further comprising replacing the second nucleobase with a fifth nucleobase that is complementary to the fourth nucleobase, thereby generating an intended edited basepair.

227. The method of any one of claims 224-226, wherein the efficiency of generating the intended edited basepair is at least 5%.

228. The method of claim 227, wherein the efficiency is at least 10%, 15%, 20%, 25%, 30%, 35%, 40%, 45%, or 50%.

229. The method of claim 226, wherein the ratio of intended products to unintended products at the target nucleotide is at least 2:1, 5:1, 10:1, 20:1, 30:1, 40:1, 50:1, 60:1, 70:1, 80:1, 90:1, 100:1, or 200:1.

230. The method of claim 226, wherein the ratio of intended point mutation to indel formation is greater than 1:1, 10:1, 50:1, 100:1, 500:1, or 1000:1.

231. The method of any one of claims 224-230, wherein the cut single strand is hybridized to the guide nucleic acid.

232. The method of any one of claims 224-231, wherein the cut single strand is opposite to the strand comprising the first nucleobase.

233. The method of any one of claims 224-232, wherein said first base is cytosine.

234. The method of any one of claims 224-233, wherein the second nucleobase is not a G, C, A, or T.

235. The method of any one of claims 224-234, wherein said second base is uracil.

236. The method of any one of claims 224-235, wherein the nucleobase editor comprises UGI activity.

237. The method of any one of claims 224-236, wherein the nucleobase editor comprises nickase activity.

238. The method of any one of claims 226-237, wherein the intended edited basepair is upstream of a PAM site.

239. The method of claim 238, wherein the intended edited base pair is 1, 2, 3, 4, 5, 6, 7, 8, 9, 10, 11, 12, 13, 14, 15, 16, 17, 18, 19, or 20 nucleotides upstream of the PAM site.

240. The method of claim 239, wherein the intended edited basepair is downstream of a PAM site.

241. The method of claim 240, wherein the intended edited base pair is 1, 2, 3, 4, 5, 6, 7, 8, 9, 10, 11, 12, 13, 14, 15, 16, 17, 18, 19, or 20 nucleotides downstream stream of the PAM site.

242. The method of any one of claims 224-241, wherein the method does not require a canonical PAM site.

243. The method of claim 242, wherein the canonical PAM sit comprises NGG, wherein N is A, T, C, or G.

244. The method of any one of claims 224-243, wherein the nucleobase editor comprises a linker.

245. The method of claim 244, wherein the linker is 1-25 amino acids in length.

246. The method of claim 244 or 245, wherein the linker is 5-20 amino acids in length.

247. The method of any one of claims 244-246, wherein the linker is 10, 11, 12, 13, 14, 15, 16, 17, 18, 19, or 20 amino acids in length.

248. The method of any one of claims 224-247, wherein the target region comprises a target window, wherein the target window comprises the target nucleobase pair.

249. The method of claim 248, wherein the target window comprises 1-10 nucleotides.

250. The method of claim 248, wherein the target window is 1-9, 1-8, 1-7, 1-6, 1-5, 1-4, 1-3, 1-2, or 1 nucleotides in length.

251. The method of claim 248, wherein the target window is 1, 2, 3, 4, 5, 6, 7, 8, 9, 10, 11, 12, 13, 14, 15, 16, 17, 18, 19, or 20 nucleotides in length.

252. The method of claim any one of claims 224-251, wherein the intended edited base pair occurs within the target window.

253. The method of claim any one of claims 224-252, wherein the target window comprises the intended edited base pair.

254. The method of any one of claims 224-253, wherein the nucleobase editor comprises any one of the fusion proteins of claims 1-189

255. A method for editing a nucleobase pair of a double-stranded DNA sequence, the method comprising:
a. contacting a target region of the double-stranded DNA sequence with a complex comprising a nucleobase editor and a guide nucleic acid, wherein the target region comprises a target nucleobase pair;
b. inducing strand separation of said target region;
c. converting a first nucleobase of said target nucleobase pair in a single strand of the target region to a second nucleobase;
d. cutting no more than one strand of said target region;
wherein a third nucleobase complementary to the first nucleobase base is replaced by a fourth nucleobase complementary to the second nucleobase; and
e. replacing the second nucleobase with a fifth nucleobase that is complementary to the fourth nucleobase, thereby generating an intended edited basepair,
wherein the efficiency of generating the intended edited basepair is at least 5%.

256. The method of claim 255, wherein the efficiency is at least 5%, 10%, 15%, 20%, 25%, 30%, 35%, 40%, 45%, or 50%.

257. The method of claim 255 or 256, wherein the method causes less than 19%, 18%, 16%, 14%, 12%, 10%, 8%, 6%, 4%, 2%, or 1% indel formation.

258. The method of any one of claims 255-257, wherein the ratio of intended product to unintended products at the target nucleotide is at least 2:1, 5:1, 10:1, 20:1, 30:1, 40:1, 50:1, 60:1, 70:1, 80:1, 90:1, 100:1, or 200:1.

259. The method of any one of claims 255-258, wherein the ratio of intended point mutation to indel formation is greater than 1:1, 10:1, 50:1, 100:1, 500:1, or 1000:1.

260. The method of any one of claims 255-259, wherein the cut single strand is hybridized to the guide nucleic acid.

261. The method of claim any one of claims 255-260, wherein the cut single strand is opposite to the strand comprising the first nucleobase.

262. The method of any one of claims 255-261, wherein said first base is cytosine.

263. The method of any one of claims 255-262, wherein the second nucleobase is not G, C, A, or T.

264. The method of any one of claims 255-263, wherein said second base is uracil.

265. The method of any one of claims 255-264, wherein the nucleobase editor comprises UGI activity.

266. The method of any one of claims 255-265, wherein the nucleobase edit comprises nickase activity.

267. The method of any one of claims 255-266, wherein the intended edited basepair is upstream of a PAM site.

268. The method of claim 267, wherein the intended edited base pair is 1, 2, 3, 4, 5, 6, 7, 8, 9, 10, 11, 12, 13, 14, 15, 16, 17, 18, 19, or 20 nucleotides upstream of the PAM site.

269. The method of any one of claims 255-266, wherein the intended edited basepair is downstream of a PAM site.

270. The method of claim 269, wherein the intended edited base pair is 1, 2, 3, 4, 5, 6, 7, 8, 9, 10, 11, 12, 13, 14, 15, 16, 17, 18, 19, or 20 nucleotides downstream stream of the PAM site.

271. The method of any one of claims 255-270, wherein the method does not require a canonical PAM site.

272. The method of claim 271, wherein the canonical PAM site comprises NGG, wherein N is A, T, C, or G.

273. The method of any one of claims 255-272, wherein the nucleobase editor comprises a linker.

274. The method of claim 273, wherein the linker is 1-25 amino acids in length.

275. The method of claim 274 or 275, wherein the linker is 5-20 amino acids in length.

276. The method of any one of claims 274-275, wherein the linker is 10, 11, 12, 13, 14, 15, 16, 17, 18, 19, or 20 amino acids in length.

277. The method of any one of claims 274-27, wherein the target region comprises a target window, wherein the target window comprises the target nucleobase pair.

278. The method of claim 277 wherein the target window comprises 1-10 nucleotides.

279. The method of claim 277, wherein the target window is 1-9, 1-8, 1-7, 1-6, 1-5, 1-4, 1-3, 1-2, or 1 nucleotides in length.

280. The method of claim 277, wherein the target window is 1, 2, 3, 4, 5, 6, 7, 8, 9, 10, 11, 12, 13, 14, 15, 16, 17, 18, 19, or 20 nucleotides in length.

281. The method of any one of claims 277-280, wherein the intended edited base pair occurs within the target window.

282. The method of any one of claims 277-281, wherein the target window comprises the intended edited base pair.

283. The method of any one of claims 255-282, wherein the nucleobase editor comprises any one of the fusion proteins of claims 1-189

284. A nucleic acid-guided deaminase coupled to an inhibitor of base excision repair.

285. The nucleic acid-guided deaminase of claim 284 comprising an initiator of mismatch repair.

286. The nucleic acid-guided deaminase of claim 284 comprising a nickase.

287. A method for editing a nucleobase pair of a double-stranded DNA sequence, the method comprising:
a. contacting a target region of the double-stranded DNA sequence with a nucleic acid-guided deaminase, wherein the target region comprises a target nucleobase pair;
b. converting a first nucleobase of said target nucleobase pair of the target region to a second nucleobase; and
c. inhibiting base excision repair of the second nucleobase.

288. The method of claim 287 further comprising nicking the non-edited strand of the target double-stranded DNA sequence.

289. The method of claim 287 further comprising initiating mismatch repair to convert the nucleobase complementary to the first nucleobase on the non-edited strand to a nucleobase complementary to the second nucleobase.

290. The method of claim 287 further comprising inducing strand separation in the target region.

291. A method for editing a nucleobase pair of a double-stranded DNA sequence, the method comprising:
a. contacting a target region of the double-stranded DNA sequence with a nucleic acid-guided deaminase, wherein the target region comprises a target nucleobase pair;
b. converting a first nucleobase of said target nucleobase pair in the target region to a second nucleobase; and
c. initiating mismatch repair to convert the nucleobase complementary to the first nucleobase on the non-edited strand to a nucleobase complementary to the second nucleobase.

292. The method of claim 291 further comprising inhibiting base excision repair of the second nucleobase.

293. The method of claim 291 further comprising inducing strand separation in the target region.

294. The method of claim 287 or 291, wherein the nucleic acid-guided deaminase is a nucleic acid-guided cytidine deaminase.

295. A kit comprising a nucleic acid construct, comprising
(a) a nucleic acid sequence encoding the fusion protein of any one of claims 1-189; and
(b) a heterologous promoter that drives expression of the sequence of (a).

296. The kit of claim 256, further comprising an expression construct encoding a guide RNA backbone, wherein the construct comprises a cloning site positioned to allow the cloning of a nucleic acid sequence identical or complementary to a target sequence into the guide RNA backbone.

297. A polynucleotide encoding the fusion protein of any one of claims 1-189.

298. A vector comprising a polynucleotide of claim 258.

299. The vector of claim 259, wherein the vector comprises a heterologous promoter driving expression of the polynucleotide.

300. A cell comprising the fusion protein of any one of claims 1-189.

301. A cell comprising the complex of any of claims 190-205.

302. A cell comprising the nucleic acid molecule encoding the fusion protein of any one of claims 1-189.
